(19) United States
(12) Patent Application Publication
(10) Pub. No.: US 2024/0002477 A1
(43) Pub. Date: Jan. 4, 2024

(54) POLYPEPTIDES FOR DETECTION AND TREATMENT OF CORONAVIRUS INFECTION

(71) Applicants: The University of Chicago, Chicago, IL (US); Wisconsin Alumni Research Foundation, Madison, WI (US)

(72) Inventors: Patrick WILSON, Chicago, IL (US); Haley DUGAN, Chicago, IL (US); Christopher STAMPER, Chicago, IL (US); Yoshihiro KAWAOKA, Madison, WI (US); Peter HALFMANN, Madison, WI (US)

(73) Assignees: The University of Chicago, Chicago, IL (US); Wisconsin Alumni Research Foundation, Madison, WI (US)

(21) Appl. No.: 18/255,609

(22) PCT Filed: Dec. 3, 2021
(86) PCT No.: PCT/US21/72723

§ 371 Date: Jun. 2, 2023

Related U.S. Application Data
(60) Provisional application No. 63/121,384, filed on Dec. 4, 2020.

(51) Int. Cl. C07K 16/10; G01N 33/569; A61P 31/14

(52) U.S. Cl. CPC C07K 16/1003; G01N 33/56983; A61P 31/14; G01N 2333/165; G01N 2469/10; C07K 2317/92; C07K 2317/76; A61K 2039/505

(57) ABSTRACT

To address the need in the art, the inventors have comprehensively characterized the SARS-CoV-2-specific B cell repertoire in convalescent COVID-19 patients and generated mAbs against the spike, ORF8, and NP proteins. Together, the inventors' data reveal key insight into antigen specificity and B cell subset distribution upon SARS-CoV-2 infection in the context of age, sex, and disease severity. Aspects of the disclosure relate to novel antibody and antigen binding fragments. Further aspects relate to polypeptides comprising the antigen binding fragment(s) of the disclosure, and compositions comprising the polypeptides, antibodies, and/or antigen binding fragments of the disclosure. Also described are nucleic acids encoding an antibody or antigen binding fragment of the disclosure.

### CROSS-REFERENCE TO RELATED APPLICATIONS

This application claims the benefit of priority of U.S. Provisional Patent Application No. 63/121,384 filed Dec. 4, 2020, which is hereby incorporated by reference in its entirety.

### STATEMENT OF GOVERNMENT SUPPORT

This invention was made with government support under contract numbers 75N93019C00062 and 75N93019C00051 awarded by the National Institutes of Health. The government has certain rights in the invention.

### SEQUENCE LISTING

The instant application contains a Sequence Listing which has been submitted in ASCII format and is hereby incorporated by reference in its entirety. Said ASCII copy, created on Nov. 24, 2021, is named ARCDP0715WO_ST25.txt and is 1,359,473 bytes in size.

### BACKGROUND

### I. Field of the Invention

Aspects of the invention relate to at least the fields of virology and molecular biology.

### II. Background

Since the emergence of SARS-CoV-2 in December 2019, the World Health Organization has reported spread to over 200 countries with infections approaching 64 million and deaths 1.5 million worldwide. Despite this burden, the quest to identify effective vaccines, therapies, and protective biomarkers continues. The isolation of human monoclonal antibodies (mAbs) specific for immunogenic SARS-CoV-2 proteins holds immense potential, as they can be rapidly employed as therapeutic agents, diagnostic reagents, and aid vaccine optimization. Several independent groups have identified potently neutralizing mAbs against the SARS-CoV-2 spike protein, the major immunogenic surface glycoprotein 1-7. Despite these advances, there have been no mAbs isolated against other immunogenic targets of SARS-CoV-2, including the internal nucleoprotein (NP) and open reading frame (ORF) protein, which have been suggested to induce antibody responses and immunomodulatory effects in humans 8-12. Moreover, the properties and frequencies of B cell subsets targeting distinct SARS-CoV-2 antigens remain poorly understood, and are likely shaped by clinical features such as age and disease severity^{6,13,14}. Therefore, there is a need in the art for effective therapies against SARS-CoV-2.

### SUMMARY

To address the need for new treatments, the inventors have comprehensively characterized the SARS-CoV-2-specific B cell repertoire in convalescent COVID-19 patients and generated mAbs against the spike, ORFS, and NP proteins. Together, the inventors' data reveal key insights into antigen specificity and B cell subset distribution upon SARS-CoV-2 infection in the context of age, sex, and disease severity. Aspects of the disclosure relate to novel antibody and antigen binding fragments, as well as methods of using these fragments. Further aspects relate to polypeptides comprising the antigen binding fragment(s) of the disclosure, and compositions comprising the polypeptides, antibodies, and/or antigen binding fragments of the disclosure. Also described are nucleic acids encoding an antibody or antigen binding fragment of the disclosure. The disclosure also relates to nucleic acids encoding an antibody heavy chain, wherein the nucleic acid has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:1621-1710 or 2707-2755. Also described are nucleic acids encoding an antibody light chain of the disclosure, wherein the nucleic acid has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:1711-1800 or 2756-2804. Further aspects relate to vectors or expression vectors comprising nucleic acids of the disclosure and host cells comprising polypeptides, nucleic acids, vectors, antibodies, or antigen binding fragments of the disclosure. The nucleic acids of the disclosure may be DNA or RNA.

Also described is a method of a making a cell comprising transferring one or more nucleic acid(s) of the disclosure into a cell. In some embodiments, the method further comprises culturing the cell under conditions that allow for expression of a polypeptide from the nucleic acid. In some embodiments, the method further comprising isolating the expressed polypeptide. The cell may be further defined as a human cell, B cell, T cell, Chinese hamster ovary, NS0 murine myeloma cell, PER.C6 cell, or a cell described herein.

Further aspects of the disclosure relate to a method for treating or preventing a coronavirus infection in a subject, the method comprising administering to the subject an antibody, antigen binding fragment, polypeptide, nucleic acid, or host cell of the disclosure. Yet further aspects relate to a method for evaluating a sample from a subject, the method comprising contacting a biological sample from the subject, or extract thereof, with at least one antibody, antigen binding fragment, or polypeptide of the disclosure. Also disclosed is a method for diagnosing a SARS-CoV-2 infection in a subject, the method comprising contacting a biological sample from the subject, or extract thereof, with at least one antibody, antigen binding fragment, or polypeptide of any one of the disclosure. In some aspects, the compositions of the disclosure are formulated as a vaccine for the treatment or prevention of a coronoavirus infection. In some embodiments, the antibodies, antigen binding fragments, or compositions of the disclosure are used in a vaccine for preventing coronaviral infections in a subject that does not have a coronaviral infection. In some embodiments, the antibodies, antigen binding fragments, or compositions of the disclosure are used to treat a subject having a coronaviral infection.

Also described is a method of a making a cell comprising transferring one or more nucleic acid(s) of the disclosure into a cell. In some aspects, the method further comprises culturing the cell under conditions that allow for expression of a polypeptide from the nucleic acid. In some aspects, the method further comprising isolating the expressed polypeptide. Aspects describe a method for producing a polypeptide comprising transferring one or more nucleic acid(s) or vector(s) of the disclosure into a cell and isolating polypeptides expressed from the nucleic acid. The cell may be further defined as a human cell, B cell, T cell, Chinese hamster ovary, NS0 murine myeloma cell, PER.C6 cell, or a cell described herein.

Further aspects of the disclosure relate to a method for treating or preventing a coronavirus infection in a subject, the method comprising administering to the subject an antibody, antigen binding fragment, polypeptide, nucleic acid, or host cell of the disclosure. Yet further aspects relate to a method for evaluating a sample from a subject, the method comprising contacting a biological sample from the subject, or extract thereof, with at least one antibody, antigen binding fragment, or polypeptide of the disclosure. Also disclosed is a method for diagnosing a SARS-CoV-2 infection in a subject, the method comprising contacting a biological sample from the subject, or extract thereof, with at least one antibody, antigen binding fragment, or polypeptide of any one of the disclosure. In some aspects, the compositions of the disclosure are formulated as a vaccine for the treatment or prevention of a coronoavirus infection. In some aspects, the antibodies, antigen binding fragments, or compositions of the disclosure are used in a vaccine for preventing coronaviral infections in a subject that does not have a coronaviral infection. In some aspects, the antibodies, antigen binding fragments, or compositions of the disclosure are used to treat a subject having a coronavirus infection.

Aspects of the disclosure relate to an antibody or antigen binding fragment comprising a heavy chain variable region and a light chain variable region, wherein the heavy chain variable region comprises a HCDR1, HCDR2, and HCDR3 from a heavy chain variable region of an antibody clone of Table 1 and wherein the light chain variable region comprises a LCDR1, LCDR2, and LCDR3 from the light chain variable region of the same clone of Table 1. Further aspects relate to an antibody or antigen binding fragment comprising a heavy chain variable region and a light chain variable region, wherein the heavy chain variable region comprises a HCDR1, HCDR2, and HCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a HCDR1, HCDR2, and HCDR3 from a heavy chain variable region of an antibody clone of Table 1 and wherein the light chain variable region comprises a LCDR1, LCDR2, and LCDR3 having or having at least 80% sequence identity or having or having at least 60, 61, 62, 63, 64, 65, 66, 67, 68, 69, 70, 71, 72, 73, 74, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of the same clone of Table 1. The HCDR1, HCDR2, HCDR3, LCDR1, LCDR2, and/or LCDR3 may be determined from the variable region sequences by methods known in the art. In some aspects, the CDR is HCDR1, HCDR2, HCDR3, LCDR1, LCDR2, and/or LCDR3 determined by the Chothia method. In some aspects, the CDR is HCDR1, HCDR2, HCDR3, LCDR1, LCDR2, and/or LCDR3 determined by the Kabat method. In some aspects, the CDR is HCDR1, HCDR2, HCDR3, LCDR1, LCDR2, and/or LCDR3 determined by the IMGT method.

Aspects of the disclosure relate to an antibody or antigen binding fragment in which the HCDR1, HCDR2, HCDR2, LCDR1, LCDR2, and LCDR3 each comprise an amino acid sequence that has at least 80% sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone. In some aspects, the HCDR1, HCDR2, HCDR2, LCDR1, LCDR2, and LCDR3 each comprise an amino acid sequence that has or has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone. In some aspects, the HCDR1, HCDR2, HCDR2, LCDR1, LCDR2, and LCDR3 each comprise the amino acid sequence of an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

Aspects of the disclosure relate to an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the HCDR1, HCR2, HCR3 from a heavy chain variable region of a antibody clone of Table 1 and wherein the light chain variable region comprises a LCDR1, LCDR2, and LCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LCDR1, LCDR2, and LCDR3 from the light chain variable region of the same antibody clone of Table 1. In some aspects, the antibody or antigen binding fragment comprises a heavy chain variable region and a light chain variable region, wherein the heavy chain variable region comprises a HCDR1, HCDR2, and HCDR3 having or having at least 80% sequence identity to the HCDR1, HCR2, HCR3 from a heavy chain variable region of a antibody clone of Table 1 and wherein the light chain variable region comprises a LCDR1, LCDR2, and LCDR3 having at least 80% sequence identity to the LCDR1, LCDR2, and LCDR3 from the light chain variable region of the same antibody clone of Table 1. In some aspects, the heavy chain variable region comprises a HCDR1, HCDR2, and HCDR3 having the amino acid sequence of an of a HCDR1, HCDR2, and HCDR3 of a clone of Table 1 and the light chain variable region comprises a LCDR1, LCDR2, and LCDR3 comprising the amino acid sequence of the LCDR1, LCDR2, and LCDR3 from the light chain variable region of the same clone of Table 1.

The polypeptides of the disclosure may comprise at least two antigen binding fragments, wherein each antigen binding fragment is independently selected from an antigen binding fragment of the disclosure. In some aspects, the polypeptide is multivalent. In some aspects, the polypeptide is multispecific. In some aspects, the polypeptide is bispecific. In some aspects, the polypeptide comprises, comprises at least, or comprises at most 1, 2, 3, 4, 5, 6, 7, 8, 9, or 10 antigen binding regions. Each antigen binding region may be independently selected from an antigen binding region of the disclosure. In some aspects, the polypeptide may have repeated units of the same antigen binding region, such as at least, at most, or exactly 1, 2, 3, 4, 5, 6, 7, 8, 9, or 10 repeated units.

In some aspects, the heavy chain variable region comprises an amino acid sequence with at least 80% sequence identity to a heavy chain variable region of an antibody clone of Table 1 and/or the light chain variable region comprises an amino acid sequence with at least 80% sequence identity to the light chain variable region of the same antibody clone of Table 1. In some aspects, the heavy chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain variable region of an antibody clone of Table 1 and/or the light chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain variable region of the same antibody clone of Table 1. In some aspects, the heavy chain variable region comprises the amino acid sequence of a heavy chain variable region of an antibody clone of Table 1 and/or the light chain variable region comprises the amino acid sequence of the same antibody clone of Table 1. In some aspects, the antibody or antigen binding fragment comprises a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence with at least 80% sequence identity to an HFR1, HFR2, HFR3, and HFR4, respectively, of an antibody clone of Table 1, and the LFR1, LFR2, LFR3, and LFR4 comprises an amino acid sequence with at least 80% sequence identity to the LFR1, LFR2, LFR3, and LFR4, respectively, of the same antibody clone of Table 1. In some aspects, the antibody or antigen binding fragment comprises a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HFR1, HFR2, HFR3, and HFR4, respectively, of an antibody clone of Table 1, and the LFR1, LFR2, LFR3, and LFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LFR1, LFR2, LFR3, and LFR4, respectively, of the same antibody clone of Table 1. In some aspects, the HFR1, HFR2, HFR3, and HFR4 comprises the amino acid sequence of an HFR1, HFR2, HFR3, and HFR4, respectively, of an antibody clone of Table 1, and the LFR1, LFR2, LFR3, and LFR4 comprises the amino acid sequence of the LFR1, LFR2, LFR3, and LFR4, respectively, of the same antibody clone of Table 1. In some aspects, the antibody or antigen binding fragment comprises a heavy chain and a light chain and wherein the heavy chain comprises an amino acid sequence with at least 70% sequence identity to a heavy chain of an antibody clone of Table 1 and the light chain comprises an amino acid sequence with at least 70% sequence identity to the light chain of the same antibody clone of Table 1. In some aspects, the antibody or antigen binding fragment comprises a heavy chain and a light chain and wherein the heavy chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain of an antibody clone of Table 1 and the light chain comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain of the same antibody clone of Table 1. In some aspects, the antibody or antigen binding fragment comprises a heavy chain and a light chain and wherein the heavy chain comprises the amino acid sequence of an antibody clone of Table 1 and the light chain comprises the amino acid sequence of the same antibody clone of Table 1.

In some aspects, the heavy chain variable region comprises a heavy chain framework region that has or has at least 80% sequence identity to a heavy chain framework region of an antibody clone of Table 1 and the light chain variable region comprises a light chain framework region that has or has at least 80% sequence identity to a light chain framework region of the same antibody clone of Table 1. In some aspects, the heavy chain variable region comprises a heavy chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain framework region of an antibody clone of Table 1 and the light chain variable region comprises a light chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a light chain framework region of the same antibody clone of Table 1.

In some aspects, the heavy chain variable region comprises at least 70% sequence identity to the heavy chain variable region of an antibody clone of Table 1 and the light chain variable region comprises at least 70% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. In some aspects, the heavy chain variable region comprises at least 75% sequence identity to the heavy chain variable region of an antibody clone of Table 1 and the light chain variable region comprises at least 75% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. In some aspects, the heavy chain variable region comprises at least 80% sequence identity to the heavy chain variable region of an antibody clone of Table 1 and the light chain variable region comprises at least 80% sequence identity to the light chain variable region of the same antibody clone of Table 1, and wherein the heavy chain and light chain comprise 100% sequence identity to each of the three heavy chain CDRs and three light chain CDRs from the same antibody clone of Table 1 In some aspects, the heavy chain variable region comprises at least 85% sequence identity to the heavy chain variable region of an antibody clone of Table 1 and the light chain variable region comprises at least 85% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. In some aspects, the heavy chain variable region comprises at least 90% sequence identity to the heavy chain variable region of an antibody clone of Table 1 and the light chain variable region comprises at least 90% sequence identity to the light chain variable region of the same antibody clone of Table 1, and wherein the heavy chain and light chain comprise 100% sequence identity to each of the three heavy chain CDRs and three light chain CDRs from the same antibody clone of Table 1. In some aspects, the heavy chain variable region comprises at least 95% sequence identity to the heavy chain variable region of an antibody clone of Table 1 and the light chain variable region comprises at least 95% sequence identity to the light chain variable region of the same antibody clone of Table 1, and wherein the heavy chain and light chain comprise 100% sequence identity to each of the three heavy chain CDRs and three light chain CDRs from the same antibody clone of Table 1.

The antibody or antigen binding fragment of the disclosure may be human, chimeric, or humanized. In some aspects, the antibody, or antigen binding fragment binds a SARS-CoV-2 Spike, NP protein, or ORF8 with a kD of about 10⁻⁶ nM to about 10⁻¹² pM. In some aspects, the antibody, or antigen binding fragment binds a SARS-CoV-2 Spike, NP protein, or ORFS with a kD of about, a kD of at least, or a kD of at most 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, or 10⁻¹⁸ (or any derivable range therein) μM, nM, or pM. In some aspects, the antibody or antigen binding fragment specifically binds to a receptor-binding domain (RBD) of the SARS-CoV-2 spike protein. The antibody may be further defined as a neutralizing antibody. In some aspects, the antibody or antigen binding fragment is further defined as a human antibody or antigen binding fragment, humanized antibody or antigen binding fragment, recombinant antibody or antigen binding fragment, chimeric antibody or antigen binding fragment, an antibody or antigen binding fragment derivative, a veneered antibody or antigen binding fragment, a diabody, a monoclonal antibody or antigen binding fragment, a single domain antibody, or a single chain antibody. In some aspects, the antigen binding fragment is further defined as a single chain variable fragment (scFv), F(ab′)2, Fab′, Fab, Fv, or rIgG. In some aspects, the antibody, antigen binding fragment, or polypeptide is operatively linked to a detectable label. Detectable labels are described herein.

Aspects of the disclosure also relate to multi-specific and/or multivalent antibodies and polypeptides. Accordingly, aspects relate to bivalent or bispecific antibodies that comprise two antigen binding fragments, wherein the antigen binding fragment is two of the same antigen binding fragments or two different antigen binding fragments described herein. The disclosure also provides for multi-specific polypeptides. Aspects relate to polypeptides comprising or comprising at least 2, 3, 4, 5, or 6 antigen binding fragments.

The antigen binding fragment may be at least 2, 3, 4, 5, or 6 scFv, F(ab′)2, Fab′, Fab, Fv, or rIgG, or combinations thereof. The polypeptide and/or antigen binding fragments of the disclosure may comprise a linker between a heavy chain and light chain variable region or between antigen binding fragments. The linker may be a flexible linker. Exemplary flexible linkers include glycine polymers (G)n, glycine-serine polymers (including, for example, (GS)n, (GSGGS-SEQ ID NO:2805)n, (G4S)n and (GGGS-SEQ ID NO:2806)n, where n is an integer of at least one. In some aspects, n is at least, at most, or exactly 1, 2, 3, 4, 5, 6, 7, 8, 9, or 10 (or any derivable range therein). Glycine-alanine polymers, alanine-serine polymers, and other flexible linkers known in the art and may be used as a linker in the polypeptides of the disclosure. Exemplary linkers can comprise or consist of GGSG (SEQ ID NO:2807), GGSGG (SEQ ID NO:2808), GSGSG (SEQ ID NO:2809), GSGGG (SEQ ID NO:2810), GGGSG (SEQ ID NO:2811), GSGSG (SEQ ID NO:2812), and the like.

In some aspects, the coronavirus infection is a SARS-CoV-2 infection. In some aspects, the coronavirus infection is a SARS-CoV infection. In some aspects, the coronavirus infection is a MERS-CoV infection. In some aspects, the coronavirus infection is a HCoV-HCoV-HKU1, HCoV-229E, or HCoV-NL63 infection.

Compositions of the disclosure, such as pharmaceutical compositions may comprise a pharmaceutical excipient, carrier, or molecule described herein. In some aspects, the composition further comprises an adjuvant or an immunostimulator. Such adjuvants or immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherichia coli, Salmonella minnesota, Salmonella typhimurium, or Shigella flexneri or specifically with MPL (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as ASO1, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments. Compositions may comprise more than one antibody and/or antigen binding fragment of the disclosure. Accordingly, compositions of the disclosure may comprise, may comprise at least, or may comprise at most 1, 2, 3, 4, 5, 6, 7, 8, 9, or 10 antibodies and/or antigen binding fragments of the disclosure. The compositions of the disclosure may be formulated for a route of administration described herein. In some aspects, the composition, antibody, antigen binding fragment, or polypeptide is formulated for parenteral, intravenous, subcutaneous, intramuscular, or intranasal administration. In a particular aspect, the compositions is formulated for intranasal administration.

In some aspects, the host cell is a human cell, B cell, T cell, Chinese hamster ovary, NS0 murine myeloma cell, or PER.C6 cell. In some aspects, the host cell is a cell type or cell population described herein.

In aspects of the disclosure, the subject or patient may be a human subject or a human patient. In some aspects, the subject or patient is a non-human animal. In some aspects, the non-human animal is a bat, monkey, camel, rat, mouse, rabbit, goat, chicken, bird, cat, or dog. The subject may further be defined as an at-risk subject. At-risk subjects include health care workers, immunocompromised subjects, people over the age of 65, or those with at least one or at least two underlying conditions. Example of underlying conditions include obesity, high blood pressure, autoimmunity, cancer, and asthma. In some aspects, the subject has one or more symptoms of a coronavirus infection. Symptoms of a coronavirus infection include, but are not limited to elevated temperature or a fever of 100.0° F. or more, loss of taste or smell, cough, difficulty breathing, shortness of breath, fatigue, headache, chills, sore throat, congestion or runny nose, shaking or exaggerated shivering, significant muscle pain or ache, diarrhea, and/or nausea or vomiting. In some aspects, the subject does not have any symptoms of a coronavirus infection. In some aspects, the subject has been diagnosed with a coronavirus infection. In some aspects, the subject has not been diagnosed with a coronavirus infection. In some aspects, the subject has been previously treated for a coronavirus infection. In some aspects, the subject has been previously vaccinated for coronavirus. In some aspects, the subject has not been previously vaccinated for coronavirus. In some aspects, the previous treatment comprises a pain reliever, such as acetaminophen or ibuprofen, a steroid such as dexamethasone, prednisolone, beclomethasone, fluticasone, or methylprednisone or an antiviral such as remdesivir. In some aspects, the subject is administered an additional therapeutic. The additional therapeutic may comprise one or more of a pain reliever, such as acetaminophen or ibuprofen, a steroid such as dexamethasone, prednisolone, beclomethasone, fluticasone, or methylprednisone or an antiviral such as remdesivir. In some aspects, the additional therapeutic comprises dexamethasone. In some aspects, the additional therapeutic comprises remdesivir.

In some aspects of the disclosure, the method further comprises incubating the antibody, antigen binding fragment, or polypeptide under conditions that allow for the binding of the antibody, antigen binding fragment, or polypeptide to antigens in the biological sample or extract thereof. In some aspects, the method further comprises detecting the binding of an antigen to the antibody, antigen binding fragment, or polypeptide. In some aspects, the method further comprises contacting the biological sample with at least one capture antibody, antigen, or polypeptide. The at least one capture antibody, antigen binding fragment, or polypeptide may be an antibody, polypeptide, or antigen binding fragment of the disclosure. In some aspects, the capture antibody is linked or operatively linked to a solid support. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. In some aspects, the biological sample comprises a blood sample, urine sample, fecal sample, or nasopharyngeal sample. In aspects of the disclosure, the at least one antibody, antigen binding fragment, or polypeptide may be operatively linked to a detectable label. In some aspects, the method further comprises incubating the antibody, antigen binding fragment, or polypeptide under conditions that allow for the binding of the antibody, antigen binding fragment, or polypeptide to antigens in the biological sample or extract thereof. In some aspects, the method further comprises detecting the binding of an antigen to the antibody, antigen binding fragment, or polypeptide. In some aspects, the method further comprises contacting the biological sample with at least one capture antibody, antigen, or polypeptide. In some aspects, the biological sample comprises a blood sample, urine sample, fecal sample, or nasopharyngeal sample.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:3-5, respectively and the LCDR1, LCDR2, and LCDR3 comprises an amino acid sequence of SEQ ID NOS:12-14, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:21-23, respectively and the LCDR1, LCDR2, and LCDR3 comprises an amino acid sequence of SEQ ID NOS:30-32, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:39-41, respectively and the LCDR1, LCDR2, and LCDR3 comprises an amino acid sequence of SEQ ID NOS:48-respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:57-59, respectively and the LCDR1, LCDR2, and LCDR3 comprises an amino acid sequence of SEQ ID NOS:66-68, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:75-77, respectively and the LCDR1, LCDR2, and LCDR3 comprises an amino acid sequence of SEQ ID NOS:84-86, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:93-95, respectively and the LCDR1, LCDR2, and LCDR3 comprises an amino acid sequence of SEQ ID NOS:102-104, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:111-113, respectively and the LCDR1, LCDR2, and LCDR3 comprises an amino acid sequence of SEQ ID NOS:120-122, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:129-131, respectively and the LCDR1, LCDR2, and LCDR3 comprises an amino acid sequence of SEQ ID NOS:138-140, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:147-149, respectively and the LCDR1, LCDR2, and LCDR3 comprises an amino acid sequence of SEQ ID NOS:156-158, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:165-167, respectively and the LCDR1, LCDR2, and LCDR3 comprises an amino acid sequence of SEQ ID NOS:174-176, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:183-185, respectively and the LCDR1, LCDR2, and LCDR3 comprises an amino acid sequence of SEQ ID NOS:192-194, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:201-203, respectively and the LCDR1, LCDR2, and LCDR3 comprises an amino acid sequence of SEQ ID NOS:210-212, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:219-221, respectively and the LCDR1, LCDR2, and LCDR3 comprises an amino acid sequence of SEQ ID NOS:228-230, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:237-239, respectively and the LCDR1, LCDR2, and LCDR3 comprises an amino acid sequence of SEQ ID NOS:246-248, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:255-257, respectively and the LCDR1, LCDR2, and LCDR3 comprises an amino acid sequence of SEQ ID NOS:264-266, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:273-275, respectively and the LCDR1, LCDR2, and LCDR3 comprises an amino acid sequence of SEQ ID NOS:282-284, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:291-293, respectively and the LCDR1, LCDR2, and LCDR3 comprises an amino acid sequence of SEQ ID NOS:300-302, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:309-311, respectively and the LCDR1, LCDR2, and LCDR3 comprises an amino acid sequence of SEQ ID NOS:318-320, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:327-329, respectively and the LCDR1, LCDR2, and LCDR3 comprises an amino acid sequence of SEQ ID NOS:336-338, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:345-347, respectively and the LCDR1, LCDR2, and LCDR3 comprises an amino acid sequence of SEQ ID NOS:354-356, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:363-365, respectively and the LCDR1, LCDR2, and LCDR3 comprises an amino acid sequence of SEQ ID NOS:372-374, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:381-383, respectively and the LCDR1, LCDR2, and LCDR3 comprises an amino acid sequence of SEQ ID NOS:390-392, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:399-401, respectively and the LCDR1, LCDR2, and LCDR3 comprises an amino acid sequence of SEQ ID NOS:408-410, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:417-419, respectively and the LCDR1, LCDR2, and LCDR3 comprises an amino acid sequence of SEQ ID NOS:426-428, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:435-437, respectively and the LCDR1, LCDR2, and LCDR3 comprises an amino acid sequence of SEQ ID NOS:444-446, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:453-455, respectively and the LCDR1, LCDR2, and LCDR3 comprises an amino acid sequence of SEQ ID NOS:462-464, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:471-473, respectively and the LCDR1, LCDR2, and LCDR3 comprises an amino acid sequence of SEQ ID NOS:480-482, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:489-491, respectively and the LCDR1, LCDR2, and LCDR3 comprises an amino acid sequence of SEQ ID NOS:498-500, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:507-509, respectively and the LCDR1, LCDR2, and LCDR3 comprises an amino acid sequence of SEQ ID NOS:516-518, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:525-527, respectively and the LCDR1, LCDR2, and LCDR3 comprises an amino acid sequence of SEQ ID NOS:534-536, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:543-545, respectively and the LCDR1, LCDR2, and LCDR3 comprises an amino acid sequence of SEQ ID NOS:552-554, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:561-563, respectively and the LCDR1, LCDR2, and LCDR3 comprises an amino acid sequence of SEQ ID NOS:570-572, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:579-581, respectively and the LCDR1, LCDR2, and LCDR3 comprises an amino acid sequence of SEQ ID NOS:588-590, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:597-599, respectively and the LCDR1, LCDR2, and LCDR3 comprises an amino acid sequence of SEQ ID NOS:606-608, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:615-617, respectively and the LCDR1, LCDR2, and LCDR3 comprises an amino acid sequence of SEQ ID NOS:624-626, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:633-635, respectively and the LCDR1, LCDR2, and LCDR3 comprises an amino acid sequence of SEQ ID NOS:642-644, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:651-653, respectively and the LCDR1, LCDR2, and LCDR3 comprises an amino acid sequence of SEQ ID NOS:660-662, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:669-671, respectively and the LCDR1, LCDR2, and LCDR3 comprises an amino acid sequence of SEQ ID NOS:678-680, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:687-689, respectively and the LCDR1, LCDR2, and LCDR3 comprises an amino acid sequence of SEQ ID NOS:696-698, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:705-707, respectively and the LCDR1, LCDR2, and LCDR3 comprises an amino acid sequence of SEQ ID NOS:714-716, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:723-725, respectively and the LCDR1, LCDR2, and LCDR3 comprises an amino acid sequence of SEQ ID NOS:732-734, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:741-743, respectively and the LCDR1, LCDR2, and LCDR3 comprises an amino acid sequence of SEQ ID NOS:750-752, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:759-761, respectively and the LCDR1, LCDR2, and LCDR3 comprises an amino acid sequence of SEQ ID NOS:768-770, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:777-779, respectively and the LCDR1, LCDR2, and LCDR3 comprises an amino acid sequence of SEQ ID NOS:786-788, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:795-797, respectively and the LCDR1, LCDR2, and LCDR3 comprises an amino acid sequence of SEQ ID NOS:804-806, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:813-815, respectively and the LCDR1, LCDR2, and LCDR3 comprises an amino acid sequence of SEQ ID NOS:822-824, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:831-833, respectively and the LCDR1, LCDR2, and LCDR3 comprises an amino acid sequence of SEQ ID NOS:840-842, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:849-851, respectively and the LCDR1, LCDR2, and LCDR3 comprises an amino acid sequence of SEQ ID NOS:858-860, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:867-869, respectively and the LCDR1, LCDR2, and LCDR3 comprises an amino acid sequence of SEQ ID NOS:876-878, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:885-887, respectively and the LCDR1, LCDR2, and LCDR3 comprises an amino acid sequence of SEQ ID NOS:894-896, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:903-905, respectively and the LCDR1, LCDR2, and LCDR3 comprises an amino acid sequence of SEQ ID NOS:912-914, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:921-923, respectively and the LCDR1, LCDR2, and LCDR3 comprises an amino acid sequence of SEQ ID NOS:930-932, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:939-941, respectively and the LCDR1, LCDR2, and LCDR3 comprises an amino acid sequence of SEQ ID NOS:948-950, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:957-959, respectively and the LCDR1, LCDR2, and LCDR3 comprises an amino acid sequence of SEQ ID NOS:966-968, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:975-977, respectively and the LCDR1, LCDR2, and LCDR3 comprises an amino acid sequence of SEQ ID NOS:984-986, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:993-995, respectively and the LCDR1, LCDR2, and LCDR3 comprises an amino acid sequence of SEQ ID NOS:1002-1004, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1011-1013, respectively and the LCDR1, LCDR2, and LCDR3 comprises an amino acid sequence of SEQ ID NOS:1020-1022, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1029-1031, respectively and the LCDR1, LCDR2, and LCDR3 comprises an amino acid sequence of SEQ ID NOS:1038-1040, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1047-1049, respectively and the LCDR1, LCDR2, and LCDR3 comprises an amino acid sequence of SEQ ID NOS:1056-1058, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1065-1067, respectively and the LCDR1, LCDR2, and LCDR3 comprises an amino acid sequence of SEQ ID NOS:1074-1076, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1083-1085, respectively and the LCDR1, LCDR2, and LCDR3 comprises an amino acid sequence of SEQ ID NOS:1092-1094, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1101-1103, respectively and the LCDR1, LCDR2, and LCDR3 comprises an amino acid sequence of SEQ ID NOS:1110-1112, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1119-1121, respectively and the LCDR1, LCDR2, and LCDR3 comprises an amino acid sequence of SEQ ID NOS:1128-1130, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1137-1139, respectively and the LCDR1, LCDR2, and LCDR3 comprises an amino acid sequence of SEQ ID NOS:1146-1148, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1155-1157, respectively and the LCDR1, LCDR2, and LCDR3 comprises an amino acid sequence of SEQ ID NOS:1164-1166, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1173-1175, respectively and the LCDR1, LCDR2, and LCDR3 comprises an amino acid sequence of SEQ ID NOS:1182-1184, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1191-1193, respectively and the LCDR1, LCDR2, and LCDR3 comprises an amino acid sequence of SEQ ID NOS:1200-1202, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1209-1211, respectively and the LCDR1, LCDR2, and LCDR3 comprises an amino acid sequence of SEQ ID NOS:1218-1220, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1227-1229, respectively and the LCDR1, LCDR2, and LCDR3 comprises an amino acid sequence of SEQ ID NOS:1236-1238, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1245-1247, respectively and the LCDR1, LCDR2, and LCDR3 comprises an amino acid sequence of SEQ ID NOS:1254-1256, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1263-1265, respectively and the LCDR1, LCDR2, and LCDR3 comprises an amino acid sequence of SEQ ID NOS:1272-1274, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1281-1283, respectively and the LCDR1, LCDR2, and LCDR3 comprises an amino acid sequence of SEQ ID NOS:1290-1292, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1299-1301, respectively and the LCDR1, LCDR2, and LCDR3 comprises an amino acid sequence of SEQ ID NOS:1308-1310, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1317-1319, respectively and the LCDR1, LCDR2, and LCDR3 comprises an amino acid sequence of SEQ ID NOS:1326-1328, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1335-1337, respectively and the LCDR1, LCDR2, and LCDR3 comprises an amino acid sequence of SEQ ID NOS:1344-1346, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1353-1355, respectively and the LCDR1, LCDR2, and LCDR3 comprises an amino acid sequence of SEQ ID NOS:1362-1364, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1371-1373, respectively and the LCDR1, LCDR2, and LCDR3 comprises an amino acid sequence of SEQ ID NOS:1380-1382, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1389-1391, respectively and the LCDR1, LCDR2, and LCDR3 comprises an amino acid sequence of SEQ ID NOS:1398-1400, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1407-1409, respectively and the LCDR1, LCDR2, and LCDR3 comprises an amino acid sequence of SEQ ID NOS:1416-1418, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1425-1427, respectively and the LCDR1, LCDR2, and LCDR3 comprises an amino acid sequence of SEQ ID NOS:1434-1436, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1443-1445, respectively and the LCDR1, LCDR2, and LCDR3 comprises an amino acid sequence of SEQ ID NOS:1452-1454, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1461-1463, respectively and the LCDR1, LCDR2, and LCDR3 comprises an amino acid sequence of SEQ ID NOS:1470-1472, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1479-1481, respectively and the LCDR1, LCDR2, and LCDR3 comprises an amino acid sequence of SEQ ID NOS:1488-1490, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1497-1499, respectively and the LCDR1, LCDR2, and LCDR3 comprises an amino acid sequence of SEQ ID NOS:1506-1508, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1515-1517, respectively and the LCDR1, LCDR2, and LCDR3 comprises an amino acid sequence of SEQ ID NOS:1524-1526, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1533-1535, respectively and the LCDR1, LCDR2, and LCDR3 comprises an amino acid sequence of SEQ ID NOS:1542-1544, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1551-1553, respectively and the LCDR1, LCDR2, and LCDR3 comprises an amino acid sequence of SEQ ID NOS:1560-1562, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1569-1571, respectively and the LCDR1, LCDR2, and LCDR3 comprises an amino acid sequence of SEQ ID NOS:1578-1580, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1587-1589, respectively and the LCDR1, LCDR2, and LCDR3 comprises an amino acid sequence of SEQ ID NOS:1596-1598, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1605-1607, respectively and the LCDR1, LCDR2, and LCDR3 comprises an amino acid sequence of SEQ ID NOS:1614-1616, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1827-1829, respectively and the LCDR1, LCDR2, and LCDR3 comprises an amino acid sequence of SEQ ID NOS:1836-1838, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1845-1847, respectively and the LCDR1, LCDR2, and LCDR3 comprises an amino acid sequence of SEQ ID NOS:1854-1856, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1863-1865, respectively and the LCDR1, LCDR2, and LCDR3 comprises an amino acid sequence of SEQ ID NOS:1872-1874, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1881-1883, respectively and the LCDR1, LCDR2, and LCDR3 comprises an amino acid sequence of SEQ ID NOS:1890-1892, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1899-1901, respectively and the LCDR1, LCDR2, and LCDR3 comprises an amino acid sequence of SEQ ID NOS:1908-1910, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1917-1919, respectively and the LCDR1, LCDR2, and LCDR3 comprises an amino acid sequence of SEQ ID NOS:1926-1928, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1935-1937, respectively and the LCDR1, LCDR2, and LCDR3 comprises an amino acid sequence of SEQ ID NOS:1944-1946, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1953-1955, respectively and the LCDR1, LCDR2, and LCDR3 comprises an amino acid sequence of SEQ ID NOS:1962-1964, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1971-1973, respectively and the LCDR1, LCDR2, and LCDR3 comprises an amino acid sequence of SEQ ID NOS:1980-1982, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:1989-1991, respectively and the LCDR1, LCDR2, and LCDR3 comprises an amino acid sequence of SEQ ID NOS:1998-2000, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2007-2009, respectively and the LCDR1, LCDR2, and LCDR3 comprises an amino acid sequence of SEQ ID NOS:2016-2018, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2025-2027, respectively and the LCDR1, LCDR2, and LCDR3 comprises an amino acid sequence of SEQ ID NOS:2034-2036, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2043-2045, respectively and the LCDR1, LCDR2, and LCDR3 comprises an amino acid sequence of SEQ ID NOS:2052-2054, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2061-2063, respectively and the LCDR1, LCDR2, and LCDR3 comprises an amino acid sequence of SEQ ID NOS:2070-2072, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2079-2081, respectively and the LCDR1, LCDR2, and LCDR3 comprises an amino acid sequence of SEQ ID NOS:2088-2090, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2097-2099, respectively and the LCDR1, LCDR2, and LCDR3 comprises an amino acid sequence of SEQ ID NOS:2106-2108, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2115-2117, respectively and the LCDR1, LCDR2, and LCDR3 comprises an amino acid sequence of SEQ ID NOS:2124-2126, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2133-2135, respectively and the LCDR1, LCDR2, and LCDR3 comprises an amino acid sequence of SEQ ID NOS:2142-2144, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2151-2153, respectively and the LCDR1, LCDR2, and LCDR3 comprises an amino acid sequence of SEQ ID NOS:2160-2162, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2169-2171, respectively and the LCDR1, LCDR2, and LCDR3 comprises an amino acid sequence of SEQ ID NOS:2178-2180, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2187-2189, respectively and the LCDR1, LCDR2, and LCDR3 comprises an amino acid sequence of SEQ ID NOS:2196-2198, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2205-2207, respectively and the LCDR1, LCDR2, and LCDR3 comprises an amino acid sequence of SEQ ID NOS:2214-2216, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2223-2225, respectively and the LCDR1, LCDR2, and LCDR3 comprises an amino acid sequence of SEQ ID NOS:2232-2234, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2241-2243, respectively and the LCDR1, LCDR2, and LCDR3 comprises an amino acid sequence of SEQ ID NOS:2250-2252, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2259-2261, respectively and the LCDR1, LCDR2, and LCDR3 comprises an amino acid sequence of SEQ ID NOS:2268-2270, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2277-2279, respectively and the LCDR1, LCDR2, and LCDR3 comprises an amino acid sequence of SEQ ID NOS:2286-2288, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2295-2297, respectively and the LCDR1, LCDR2, and LCDR3 comprises an amino acid sequence of SEQ ID NOS:2304-2306, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2313-2315, respectively and the LCDR1, LCDR2, and LCDR3 comprises an amino acid sequence of SEQ ID NOS:2322-2324, respectively.

Aspects of the disclosure relate to an antibody, antigen binding fragment, or

polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2331-2333, respectively and the LCDR1, LCDR2, and LCDR3 comprises an amino acid sequence of SEQ ID NOS:2340-2342, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2349-2351, respectively and the LCDR1, LCDR2, and LCDR3 comprises an amino acid sequence of SEQ ID NOS:2358-2360, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2367-2369, respectively and the LCDR1, LCDR2, and LCDR3 comprises an amino acid sequence of SEQ ID NOS:2376-2378, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2385-2387, respectively and the LCDR1, LCDR2, and LCDR3 comprises an amino acid sequence of SEQ ID NOS:2394-2396, respectively.

Aspects of the disclosure relate to an antibody, antigen binding fragment, or

polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2403-2405, respectively and the LCDR1, LCDR2, and LCDR3 comprises an amino acid sequence of SEQ ID NOS:2412-2414, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2421-2423, respectively and the LCDR1, LCDR2, and LCDR3 comprises an amino acid sequence of SEQ ID NOS:2430-2432, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2439-2441, respectively and the LCDR1, LCDR2, and LCDR3 comprises an amino acid sequence of SEQ ID NOS:2448-2450, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2457-2459, respectively and the LCDR1, LCDR2, and LCDR3 comprises an amino acid sequence of SEQ ID NOS:2466-2468, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2475-2477, respectively and the LCDR1, LCDR2, and LCDR3 comprises an amino acid sequence of SEQ ID NOS:2484-2486, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2493-2495, respectively and the LCDR1, LCDR2, and LCDR3 comprises an amino acid sequence of SEQ ID NOS:2502-2504, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2511-2513, respectively and the LCDR1, LCDR2, and LCDR3 comprises an amino acid sequence of SEQ ID NOS:2520-2522, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2529-2531, respectively and the LCDR1, LCDR2, and LCDR3 comprises an amino acid sequence of SEQ ID NOS:2538-2540, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2547-2549, respectively and the LCDR1, LCDR2, and LCDR3 comprises an amino acid sequence of SEQ ID NOS:2556-2558, respectively.

Aspects of the disclosure relate to an antibody, antigen binding fragment, or

polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2565-2567, respectively and the LCDR1, LCDR2, and LCDR3 comprises an amino acid sequence of SEQ ID NOS:2574-2576, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2583-2585, respectively and the LCDR1, LCDR2, and LCDR3 comprises an amino acid sequence of SEQ ID NOS:2592-2594, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2601-2603, respectively and the LCDR1, LCDR2, and LCDR3 comprises an amino acid sequence of SEQ ID NOS:2610-2612, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2619-2621, respectively and the LCDR1, LCDR2, and LCDR3 comprises an amino acid sequence of SEQ ID NOS:2628-2630, respectively.

Aspects of the disclosure relate to an antibody, antigen binding fragment, or

polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2637-2639, respectively and the LCDR1, LCDR2, and LCDR3 comprises an amino acid sequence of SEQ ID NOS:2646-2648, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2655-2657, respectively and the LCDR1, LCDR2, and LCDR3 comprises an amino acid sequence of SEQ ID NOS:2664-2666, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2673-2675, respectively and the LCDR1, LCDR2, and LCDR3 comprises an amino acid sequence of SEQ ID NOS:2682-2684, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:2691-2693, respectively and the LCDR1, LCDR2, and LCDR3 comprises an amino acid sequence of SEQ ID NOS:2700-2702, respectively.

Aspects of the disclosure relate to an antibody, antigen binding fragment, or polypeptide comprising a heavy chain variable region and a light chain variable region of SEQ ID NOS:2 and 11, SEQ ID NOS:20 and 29, SEQ ID NOS:38 and 47, SEQ ID NOS:56 and 65, SEQ ID NOS:74 and 83, SEQ ID NOS:92 and 101, SEQ ID NOS:110 and 119, SEQ ID NOS:128 and 137, SEQ ID NOS:146 and 155, SEQ ID NOS:164 and 173, SEQ ID NOS:182 and 191, SEQ ID NOS:200 and 209, SEQ ID NOS:218 and 227, SEQ ID NOS:236 and 245, SEQ ID NOS:254 and 263, SEQ ID NOS:272 and 281, SEQ ID NOS:290 and 299, SEQ ID NOS:308 and 317, SEQ ID NOS:326 and 335, SEQ ID NOS:344 and 353, SEQ ID NOS:362 and 371, SEQ ID NOS:380 and 389, SEQ ID NOS:398 and 407, SEQ ID NOS:416 and 425, SEQ ID NOS:434 and 443, SEQ ID NOS:452 and 461, SEQ ID NOS:470 and 479, SEQ ID NOS:488 and 497, SEQ ID NOS:506 and 515, SEQ ID NOS:524 and 533, SEQ ID NOS:542 and 551, SEQ ID NOS:560 and 569, SEQ ID NOS:578 and 587, SEQ ID NOS:596 and 605, SEQ ID NOS:614 and 623, SEQ ID NOS:632 and 641, SEQ ID NOS:650 and 659, SEQ ID NOS:668 and 677, SEQ ID NOS:686 and 695, SEQ ID NOS:704 and 713, SEQ ID NOS:722 and 731, SEQ ID NOS:740 and 749, SEQ ID NOS:758 and 767, SEQ ID NOS:776 and 785, SEQ ID NOS:794 and 803, SEQ ID NOS:812 and 821, SEQ ID NOS:830 and 839, SEQ ID NOS:848 and 857, SEQ ID NOS:866 and 875, SEQ ID NOS:884 and 893, SEQ ID NOS:902 and 911, SEQ ID NOS:920 and 929, SEQ ID NOS:938 and 947, SEQ ID NOS:956 and 965, SEQ ID NOS:974 and 983, SEQ ID NOS:992 and 1001, SEQ ID NOS:1010 and 1019, SEQ ID NOS:1028 and 1037, SEQ ID NOS:1046 and 1055, SEQ ID NOS:1064 and 1073, SEQ ID NOS:1082 and 1091, SEQ ID NOS:1100 and 1109, SEQ ID NOS:1118 and 1127, SEQ ID NOS:1136 and 1145, SEQ ID NOS:1154 and 1163, SEQ ID NOS:1172 and 1181, SEQ ID NOS:1190 and 1199, SEQ ID NOS:1208 and 1217, SEQ ID NOS:1226 and 1235, SEQ ID NOS:1244 and 1253, SEQ ID NOS:1262 and 1271, SEQ ID NOS:1280 and 1289, SEQ ID NOS:1298 and 1307, SEQ ID NOS:1316 and 1325, SEQ ID NOS:1334 and 1343, SEQ ID NOS:1352 and 1361, SEQ ID NOS:1370 and 1379, SEQ ID NOS:1388 and 1397, SEQ ID NOS:1406 and 1415, SEQ ID NOS:1424 and 1433, SEQ ID NOS:1442 and 1451, SEQ ID NOS:1460 and 1469, SEQ ID NOS:1478 and 1487, SEQ ID NOS:1496 and 1505, SEQ ID NOS:1514 and 1523, SEQ ID NOS:1532 and 1541, SEQ ID NOS:1550 and 1559, SEQ ID NOS:1568 and 1577, SEQ ID NOS:1586 and 1595, SEQ ID NOS:1604 and 1613, SEQ ID NOS:1826 and 1835, SEQ ID NOS:1844 and 1853, SEQ ID NOS:1862 and 1871, SEQ ID NOS:1880 and 1889, SEQ ID NOS:1898 and 1907, SEQ ID NOS:1916 and 1925, SEQ ID NOS:1934 and 1943, SEQ ID NOS:1952 and 1961, SEQ ID NOS:1970 and 1979, SEQ ID NOS:1988 and 1997, SEQ ID NOS:2006 and 2015, SEQ ID NOS:2024 and 2033, SEQ ID NOS:2042 and 2051, SEQ ID NOS:2060 and 2069, SEQ ID NOS:2078 and 2087, SEQ ID NOS:2096 and 2105, SEQ ID NOS:2114 and 2123, SEQ ID NOS:2132 and 2141, SEQ ID NOS:2150 and 2159, SEQ ID NOS:2168 and 2177, SEQ ID NOS:2186 and 2195, SEQ ID NOS:2204 and 2213, SEQ ID NOS:2222 and 2231, SEQ ID NOS:2240 and 2249, SEQ ID NOS:2258 and 2267, SEQ ID NOS:2276 and 2285, SEQ ID NOS:2294 and 2303, SEQ ID NOS:2312 and 2321, SEQ ID NOS:2330 and 2339, SEQ ID NOS:2348 and 2357, SEQ ID NOS:2366 and 2375, SEQ ID NOS:2384 and 2393, SEQ ID NOS:2402 and 2411, SEQ ID NOS:2420 and 2429, SEQ ID NOS:2438 and 2447, SEQ ID NOS:2456 and 2465, SEQ ID NOS:2474 and 2483, SEQ ID NOS:2492 and 2501, SEQ ID NOS:2510 and 2519, SEQ ID NOS:2528 and 2537, SEQ ID NOS:2546 and 2555, SEQ ID NOS:2564 and 2573, SEQ ID NOS:2582 and 2591, SEQ ID NOS:2600 and 2609, SEQ ID NOS:2618 and 2627, SEQ ID NOS:2636 and 2645, SEQ ID NOS:2654 and 2663, SEQ ID NOS:2672 and 2681, or SEQ ID NOS:2690 and 2699.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

“Individual, “subject,” and “patient” are used interchangeably and can refer to a human or non-human.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1a-g: B cell subsets enriched for SARS-CoV-2-reactivity are revealed by transcriptome, Ig repertoire, and probe binding. a, Model demonstrating antigen probe preparation and representative gating strategy for sorting antigen-positive B cells. b, Percentage of antigen-probe-positive total B cells (CD19⁺CD3⁻), naïve B cells (CD27⁺CD37^{int}), and memory B cells (CD27⁺CD38^{int}) (left), and naïve vs. memory B cells by subject (right; n=17 subjects yielding quality sequencing data). Statistics are paired non-parametric Friedman test (*p=0.0491; ****p<0.0001; bars=median). c, Integrated transcriptional UMAP analysis of distinct B cell clusters and the corresponding number of B cells per cluster. d, Feature library enrichment of antigen-probe-positive B cells by cluster. e, Percent probe reactivity of all B cells per cluster. f, Ig isotype usage and VH gene SHIM for all antigen-positive B cells per cluster. Bars indicate median with interquartile range. g, Representative visualization of antigen reactivity revealing antigen-specific B cells. Axes indicate antigen probe intensities.

FIG. 2a-d: Transcriptional analysis distinguishes naïve, innate-like and MBC subsets specific to SARS-CoV-2 proteins. a-b, Trajectory (a) and pseudotime (b) analyses of clusters 0-11 reveals least to most differentiated clusters. Cluster 12 is excluded from trajectory analysis as it represents a separate partition as defined by Monocle3. c, Heatmap showing the top twenty most differentially expressed genes per cluster. Red stars denote genes used in memory B cell (MBC) identification. d, Volcano plots comparing differentially expressed genes in MBC-like clusters relative to cluster 2 (näive B cells). Genes used in MBC identification are indicated: cd27, cd38, hhex, zeb2, pou2afl, spib, cd80, cd86, mcl1, prdm1, abp1, manf, bach2, pax5. Red-colored dots represent a log fold change in expression >0.1 and an adj-p value <0.01. Putative B cell subset identities are highlighted where they could be clearly defined (a).

FIG. 3a-p: SARS-CoV-2-reactive B cells exhibit unique features for isotype, SHM, subset of origin, and VH gene usage. a-1, Ig isotype, VH gene SHM, and distribution of B cells by integrated cluster for spike—(a, b, c, d), NP—(e, f, g, h) and ORFS-specific B cells (i, j, k, 1). m-p, Tree maps showing frequency of VH gene locus usage for total spike (including RBD) (m), RBD only (n), NP (o), and ORFS-specific B cells (p). Numbers in the center of each pie chart and below each tree map indicate number of cells analyzed per reactivity.

FIG. 4a-d: Characterization of mAbs from single SARS-CoV-2-reactive B cells. a, Cluster origin of cloned mAbs (n=90). b, Representative plot showing the selection of B cells chosen to clone mAbs, antigen binding curves by ELISA for each reactive mAb (spike, n=38; RBD, n=36; NP, n=19; ORF8, n=24), and percentages of total cloned mAbs exhibiting specificity (right). Dashed line on ELISA curves represents the OD₄₀₅ cutoff of 0.5 for positivity. c, Neutralization potency (log₁₀ PFU/ml) of mAbs (n=80) tested by live SARS-CoV-2 virus plaque assay. Dashed line at x=6.5 indicates cutoff for neutralization. d, Percentage of total spike, NP, and ORF8-specific mAbs that displayed neutralization activity. Numbers below each bar chart indicate the number of mAbs tested for neutralization. ELISA data are representative of 2-4 independent experiments performed in duplicate and mAbs were screened once for neutralization ability.

FIG. 5a-i: B cell antigen targeting, subset distribution, and adaptability is linked to clinical features. a, Total serum anti-Ig endpoint titers for SARS-CoV-2 antigens determined by ELISA (n=25 subjects). b, Number of IgG/IgA antibody secreting cells (ASCs) per 10⁶ cells determined by ELISpot (n=23 subjects). c. Percentage of antigen-probe-positive cells by subject. d, Percentage of antigen-probe-positive cells stratified by age (years), sex, and symptom duration (weeks). e, Two-sided spearman correlation between percentage of all cells specific to ORF8 and subject age with p and r values indicated. f, Percentage of antigen-probe-positive B cells in MBC-like clusters (3, 4, 5, 6, 7, 9, and 12) or naïve and innate-like clusters (0, 1, 2, 8, 10, 11) stratified by age, sex, and symptom duration. (g-i) VH gene SHM for antigen-specific cells from a given age (g), sex (h), or symptom duration group (i). Data in a and b were analyzed using paired non-parametric Friedman tests with multiple comparisons against the spike (*p=0.0154, ****p<0.0001; bars=median). Red dashed line in a at y=45 indicates cutoff for no serum titer detected. The data in d and f were analyzed using two-sided Chi-square or Fisher's exact tests, (****p<0.0001; ***p=0.0009). Data in g were analyzed using unpaired non-parametric Kruskal-Wallis with multiple comparisons (****p<0.0001; ***p=0.0002; bars=mean). Statistics used in h and i are two-sided unpaired non-parametric Mann-Whitney tests (****p<0.0001; bars=mean). Numbers below each bar chart indicate the number of cells analyzed.

FIG. 6a-c. Additional characteristics of B cells comprising integrated clusters. a, Antigen-probe-positive B cell distribution across integrated clusters by subject with the number of cells per subject indicated. b, Variable gene segment usage in B cell receptor heavy chains of antigen-probe-positive B cells across integrated clusters. c, Diagrams showing antigen-probe-positive B cells per cluster with probe intensities for the indicated antigens plotted on the axes.

FIG. 7. Expression of MBC and LLPC gene markers in integrated clusters. Normalized expression levels of the indicated genes represented as violin plots.

FIG. 8a-i. Heavy and light chain features of SARS-CoV-2 reactive B cells. a-b, Heavy chain (HC, a) and light chain (LC, b) complementarity determining region 3 (CDR3) lengths, shown by antigen-reactivity. c-d, HC (c) and LC (d) isoelectric points pI, shown by antigen-reactivity. e, Number of light chain (LC) somatic hypermutations (SHM), shown by antigen-reactivity. f-i. Tree maps showing frequency of Vk/L gene locus usage for spike—(f), RBD—(g), NP—(h), and ORF8-specific B cells (i). White squares indicate unique Vk/L usages. In panels a-e groups were compared by unpaired nonparametric Kruskal-Wallis test with multiple comparisons (N.S.=not significant, ****p<0.0001; ***p=0.0006; **p=0.0033). For all analyses shown, n=531 for spike, n=47 for RBD, n=293 for NP, and n=463 cells selected for ORF8.

FIG. 9a-g. Additional features of mAbs cloned from antigen-specific and multi-probe binding B cells. a, ELISA KD for specific mAbs against the spike (n=38), RBD (n=36), ORF8 (n=24), and NP (n=19), versus normalized probe intensity for spike, ORF8, and NP respectively. Whole spike antigen probe intensities are plotted for RBD-binding mAbs. Statistics are two-sided Spearman correlations with p and r values indicated. b, Example selection of multi-probe-reactive B cells. c, Isotype frequencies of multi-probe-reactive B cells. d, Number of VH gene SIAM for multi-probe-reactive B cells. e, Proportion of multi-probe-reactive B cells in integrated clusters. f, Percentage of multi-probe-reactive B cells binding PE-SA-oligo by ELISA. g, Percent multi-probe-reactive B cells exhibiting polyreactivity, as determined by ELISA. Numbers in the center of each pie chart indicate number of B cell s/mAbs analyzed.

FIG. 10a-e. SARS-CoV-2-specific B cells constitute multiple distinct clusters. (a) Model demonstrating antigen probe preparation and representative gating strategy for sorting antigen-positive B cells. (b) Integrated transcriptional UMAP analysis of distinct B cell clusters (n=42 samples from severe acute [n=10], convalescent visit 1 [n=28], and convalescent visit 2 [n=4] cohorts; 55,656 cells). (c) Cluster quality score determined by ROGUE analysis. (d) UMAP projections showing antigen-specific cells used in all downstream analyses and the clusters they derive from. (e) Quantitative visualization of antigen-specific cells and their distributions across distinct clusters.

FIG. 11a-c. B cell receptor and transcriptional analysis reveals cluster identities. (a) B cell receptor isotype usage, somatic hypermutation (SHM), and antigen reactivity by cluster for all integrated samples. SHM data are plotted with the overlay indicating the median with interquartile range. (b) Heatmap displaying differentially expressed genes across clusters. A summary of cluster identities is provided below. (c) UMAP projections with cell color indicating gene module scoring for the indicated B cell subsets. Also see Tables S5 and S6.

FIG. 12a j. B cell immunodominance and adaptability landscapes vary in acute infection in convalescence. (a) UMAP projection showing cells colored by time point of blood draw. Sev acute, severe acute; Cony v1, convalescent visit 1; Cony v2, convalescent visit 2. (b) UMAP projections showing cells binding the specified antigens, colored by time point of blood draw. (c) Percentage of B cells targeting distinct antigens by cohort. Four Cony v1 and Cony v2 subjects represent matched visits. (d-f) Quantification of B cell subsets targeting distinct antigens across cohorts. Also see FIG. 11B, bottom for clusters used to define B cell subsets. Numbers above bars indicate the number of specific cells isolated. (g) Percentage of total antigen-specific memory B cells from ˜1.5-4.5 months (mo) post-symptom onset in four matched-convalescent subjects. Statistics are chi-square test, ****p<0.0001. (h) Variable heavy-chain (VH) somatic hypermutation (SHM) of antigen-specific B cells across both convalescent time points for four matched subjects. Statistics are unpaired non-parametric Mann-Whitney tests, **p=0.0021 and ****p<0.0001. (i and j) Antigen-specific memory B cells divided by SHM tertiles at Cony v1 (I) and Cony v2 time points (J) for four matched subjects.

FIG. 13a-f. B cells targeting distinct antigens display unique variable gene usages. (a-e) Heatmaps showing the frequency of heavy- and light-chain gene pairings for B cells binding the indicated antigens using integrated data from all cohorts (left; legend indicates number of cells per pairing), and dendrograms showing the top ten variable heavy-chain (VH) gene usages for Cony v1 (n=28) and Cony v2 (n=4) cohorts (right). The number of cells encompassing the top ten VH genes represented per antigen is indicated below each dendrogram. (f) Circos plots showing the top ten heavy- and light-chain gene pairings shared across four matched Cony v1 (left; n=1,293 cells) and Cony v2 (right; n=1,438 cells) subjects. Total antigen-specific cells against SARS2 spike and RBD, HCoV spike, ORF8, and NP are shown.

FIG. 14a-h. Neutralization capacity and in vivo protective ability of mAbs to the SARS-CoV-2 spike and intracellular proteins. (a) Antigen binding curves by ELISA for antigen-specific mAbs. Dashed line at y=0.5 on ELISA curves represents the OD405 cutoff of 0.5 for positivity (spike, n=43; NP, n=19; ORF8, n=24). Data are representative of two or three independent experiments. Also see Table S7. (b) Neutralization potency (log 10 PFU/ml) of mAbs tested by SARS-CoV-2 virus plaque assay. RBD, n=33; spike non-RBD, n=13; NP, n=18; ORF8, n=24. Dashed line at x=6.5 indicates the cutoff for neutralization. Statistics are non-parametric Kruskal-Wallis with Dunn's post-test for multiple comparisons, ****p<0.0001. Data are representative of one independent experiment. (c) Weight change in hamsters intranasally challenged with SARS-CoV-2, followed by therapeutic intraperitoneal (i.p.) administration of anti-RBD antibodies (mean±SD, n=4 biological replicates for each mAb). Control conditions are PBS injection or injection of an irrelevant Ebola virus anti-GP133 mAb. (d) Viral titers of SARS-CoV-2 in lungs harvested from hamsters post-challenge in (c). Bars indicate mean±SD. Statistics are unpaired non-parametric Kruskal-Wallis with Dunn's post-test for multiple comparisons, *p=0.0135, ***p=0.0011, and **p=0.0075. (e) Weight change of mice intranasally challenged with SARS-CoV-2, followed by therapeutic i.p. administration of anti-ORF8 antibody cocktails (mean±SD, n=3 biological replicates for each mAb). (f) Viral titers of SARS-CoV-2 in lungs harvested from mice post-challenge in (e). Titers are presented as N gene copy number compared with a standard curve, and bars indicate mean±SD. Statistics performed are non-parametric Kruskal-Wallis with Dunn's post-test for multiple comparisons; no differences were significant. (g) Weight change in hamsters intranasally challenged with SARS-CoV-2, followed by therapeutic intraperitoneal (i.p.) administration of an anti-NP antibody (mean±SD, n=4 biological replicates for each mAb). (h) Viral titers of SARS-CoV-2 in lungs harvested from hamsters post-challenge shown in (g). Bars indicate mean±SD. Statistics performed are non-parametric Mann-Whitney test; no differences were significant.

FIG. 15a-n. Antigen-specificity and B cell subset distribution is linked to clinical features. (a) Reactivity distribution of total antigen-specific B cells by subject for the convalescent visit 1 cohort (n=28). (b-d) Reactivity distribution of total antigen-specific B cells by age (b), disease severity (c), and sex (d). Statistics are chi-square post hoc tests with Holm-Bonferroni adjustment, **p=0.0012 and ****p<0.0001; n.s., not significant. For age groups, 19-35 years, n=1,382 cells, 8 subjects; 36-49 years, n=5,319 cells, 13 subjects; 50-years, n=1,813 cells, 7 subjects. For severity groups, mild, n=990 cells, 4 subjects; moderate, n=4,462 cells, 13 subjects; severe, n=3,062 cells, 11 subjects. For sex, women, n=5,005 cells, 14 subjects; men, n=3,509 cells, 14 subjects. (e) Reactivity of antigen-specific memory B cells (MBCs; top) or naive B cells (bottom) by age group. Statistics are chi-square post hoc tests with Holm-Bonferroni adjustment, *p=0.0145 and ****p<0.0001; n.s., not significant. (f) Reactivity of antigen-specific MBCs (top) or naive B cells (bottom) by disease severity. Statistics are chi-square post hoc tests with Holm-Bonferroni adjustment, *p=0.0143 and ****p<0.0001; n.s., not significant. (g) Variable heavy-chain (VH) somatic hypermutation (SHM) for MBCs by age group (overlay shows median with interquartile range). Statistics are unpaired non-parametric ANOVA with Tukey's test for multiple comparisons, **p=0.002, ***p=0.0008, and ****p<0.0001. (h j) Antigen-specific MBCs by age, divided by SHM tertiles. (k) B cell subset distribution by subject. (1-n) B cell subset distribution by age (1), disease severity (m), and sex (n). Statistics are chi-square post hoc tests with Holm-Bonferroni adjustment, ***p=0.0007 and ****p<0.0001; n.s., not significant. For each group, n is the same as in (b)-(d).

FIG. 16a-d. B cell cluster distribution and antigen specificity by subject, Related to FIG. 10. (a-b) Overall cluster distribution (top) and antigen-specificity distribution (bottom) for subjects sorted with SARS2 spike (S), SARS2 RBD, NP, and ORF8 antigens, with (a) or without (b) an endemic HCoV cocktail comprised of S proteins from 229E, NL63, OC43, and HKU1 strains. (c) Integrated UMAP analysis showing cluster distribution for two severe acute subjects (R3 and R6) at pooled early (days 0, 1, 3) and late (days 7, 14) sampling time points post-convalescent plasma therapy (left) and summary of cluster distribution per timepoint (right). (d) Distribution in antigen-reactivity for pooled early and late timepoints post-convalescent plasma therapy for severe acute subjects R3 and R6. Statistics are Chi square test, n.s.=not significant.

FIG. 17a-d. Expression maps of select genes, Related to FIG. 11. (a-d) UMAP projections with cells colored by expression level of indicated genes associated with naïve B cells (a), memory B cells b), antibody-secreting cells (c), and mucosal homing (d). Also see Table S6.

FIG. 18a-j. Further analysis of antigen-specific B cell properties across distinct cohorts and timepoints, Related to FIG. 12. (a-c) Variable heavy chain (VH) somatic hypermutation (SHM) by antigen-specific B cells shown for severe acute (a; n=10), Cony v1 (b, n=28), or Cony v2 subjects (c; n=4). Overlay shows median with interquartile range. (d) Distribution of memory B cell specificity across visit timepoints for four matched Cony v1 and Cony v2 subjects, sampled at approximately 1.5 and 4.5 months post-symptom onset. Also see Table S1 for sampling time. (e-g) B cell receptor isotype usage by antigen-specific B cells shown for severe acute (e; n=10), Cony v1 (f; n=28), or Cony v2 subjects (g; n=4). (h-j) Total anti-immunoglobulin (Ig) serum titers across timepoints for 16 matched convalescent subjects, shown for SARS2 spike (h), NP (i), and ORF8 antigens (j). Dashed line at y=45 indicates cutoff for positivity; values are staggered in (j) to avoid overlap. Statistics are paired non-parametric Wilcoxon test, *p=0.0386. Data are representative of two independent experiments.

FIG. 19a-f. Correlation between antigen-probe positive B cells and serum titers, Related to FIG. 12. (a) Matched total anti-immunoglobulin (Ig) serum titers against spike, NP, and ORF8 antigens for Cony v1 subjects (n=28). Statistics are paired non-parametric Friedman test with Dunn's post-test for multiple comparisons, ****p<0.0001; ***p=0.0002; n.s.=not significant. Data are representative of two independent experiments. (b) Matched antigen-specific probe hit per Cony v1 subject (n=28). Statistics are paired non-parametric Friedman test with Dunn's post-test for multiple comparisons, n.s.=not significant. (c-e) Percentage of B cells specific for spike (d), NP (e), or ORF8 (f) in Cony v1 subjects (n=28) compared to serum titer levels for the same antigen. Statistics are nonparametric Spearman correlation, two-tailed, CI=95%, n.s.=not significant. Data are representative of two independent experiments. (f) MAbs cloned from non-specific multi-reactive B cells tested for polyreactivity (left) and PE-SA binding (right) by ELISA (n=10). Data are representative of one independent experiment.

FIG. 20a-d. Additional analyses of antigen reactivity by clinical parameter, Related to FIG. 15. (a) Percentages of antigen-specific memory B cells (MBCs) shown per Cony v1 subject by age. Age increases left to right along the graph. (b) Percentage of MBCs specific for ORF8 versus age for female (F) Cony V1 subjects (n=14). Statistics are nonparametric Spearman correlation, two-tailed, CI=95%. P value is indicated. (c) Percentage of MBCs specific for ORF8 versus age for male (M) Cony V1 subjects (n=14). Statistics are nonparametric Spearman correlation, two-tailed, CI=95%. P value is indicated, n.s.=not significant. (d) Percentages of antigen specific naïve-like B cells shown for each Cony v1 subject by severity. Severity score increases left to right along the graph, also see Table S1 for severity score per subject.

### DETAILED DESCRIPTION OF THE INVENTION

Discovery of durable memory B cell (MBC) subsets against neutralizing viral epitopes is critical for determining immune correlates of protection from SARS-CoV-2 infection. Here, the inventors identified functionally distinct SARS-CoV-2-reactive B cell subsets by profiling the repertoire of convalescent COVID-19 patients using a high-throughput B cell sorting and sequencing platform. Utilizing barcoded SARS-CoV-2 antigen baits, the inventors isolated thousands of B cells that segregated into discrete functional subsets specific for the spike, nucleocapsid protein (NP), and open reading frame (ORF) proteins 7a and 8. Spike-specific B cells were enriched in canonical MBC clusters, and monoclonal antibodies (mAbs) from these cells were potently neutralizing. By contrast, B cells specific to ORF8 and NP were enriched in naïve and innate-like clusters, and mAbs against these targets were exclusively non-neutralizing. Finally, the inventors identified that B cell specificity, subset distribution, and affinity maturation were impacted by clinical features such as age, sex, and symptom duration. Together, the data provide a comprehensive tool for evaluating B cell immunity to SARS-CoV-2 infection or vaccination and highlight the complexity of the human B cell response to SARS-CoV-2.

### I. Antibodies

Aspects of the disclosure relate to antibodies, antigen binding fragments thereof, or polypeptides capable of specifically binding to a SARS-CoV-2 spike (S) protein, NP protein, or ORFS. Certain aspects relate to antibodies, or fragments thereof, that specifically bind to a receptor binding domain (RBD) of a SARS-CoV-2 spike protein.

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bispecific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1, CDR-L2, and CDR-L3. The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1, CDR-H2 and CDR-H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:

1) Computational predictions of the tertiary structure of the antibody/epitope binding interactions based on the chemical nature of the amino acid sequence of the antibody variable region and composition of the epitope.

2) Hydrogen-deuterium exchange and mass spectroscopy

3) Polypeptide fragmentation and peptide mapping approaches in which one generates multiple overlapping peptide fragments from the full length of the polypeptide and evaluates the binding affinity of these peptides for the epitope.

4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

B. Functional Antibody Fragments and Antigen-Binding Fragments

1. Antigen-Binding Fragments

Certain aspects relate to antibody fragments, such as antibody fragments that bind to a SARS-CoV-2 spike protein. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment (also “Fab”) means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

2. Fragment Antigen Binding Region, Fab

Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature.

3. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

C. Polypeptides with antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A] [B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≥10⁻⁸ M. The antibody specifically binds antigen with “high affinity” when the KD is ≥5×10⁻⁹ M, and with “very high affinity” when the KD is ≤5×10⁻¹⁰ M.

2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

3. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

E. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the invention to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an anti sense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### II. Antibody Production

A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Anti sera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as β-interferon, IL-2, or IL-12, or genes encoding proteins involved in immune helper functions, such as B-7.A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of human antibodies include, but are not limited to, detect a cell expressing an anticipated protein, either in vivo or in vitro, pharmaceutical preparations containing the antibodies of the present invention, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and γ) and κ light chain immunoglobulin sequences, together with targeted mutations that inactivate the endogenous μ and κ chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG κ monoclonal antibodies (Lonberg et al., supra; Lonberg and Huszar, Intern. Ref. Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MPL- and msk-receptors using such an approach.

C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the invention are proteins that are structurally similar to an antibody displaying a desired biological activity, such as the ability to bind a protein, but have one or more peptide linkages optionally replaced by a linkage selected from: —CH2NH—, —CH2S—, —CH2—CH2—, —CH═CH— (cis and trans), —COCH2—, —CH(OH)CH2—, and —CH2SO— by methods well known in the art. Systematic substitution of one or more amino acids of a consensus sequence with a D-amino acid of the same type (e.g., D-lysine in place of L-lysine) may be used in certain embodiments of the invention to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### III. Obtaining Encoded Antibodies

In some aspects, there are nucleic acid molecule encoding antibody polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

2. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAF dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

4. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### IV. Viruses

Aspects of the present disclosure relate to treatment, analysis, or use of a virus. In some embodiments, disclosed are methods for treatment or prevention of a viral infection. In some embodiments, disclosed are compositions comprising one or more anti-viral agents. In some embodiments, disclosed are methods for diagnosis of a viral infection. In some embodiments, disclosed are methods for detection of a virus in a sample.

A. Coronaviruses

In particular embodiments, the virus is from the family Coronaviridae. Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and approximately 40 species. They are enveloped viruses having a positive-sense single-stranded RNA genome and a nucleocapsid having helical symmetry. The genome size of coronaviruses ranges from approximately 26-32 kilobases.

The present disclosure encompasses treatment or prevention of infection of any virus in the Coronaviridae family. In certain embodiments, the disclosure encompasses treatment or prevention of infection of any virus in the subfamily Coronavirinae and including the four genera, Alpha-, Beta-, Gamma-, and Deltacoronavirus. In specific embodiments, the disclosure encompasses treatment or prevention of infection of any virus in the genus of Betacoronavirus, including the subgenus Sarbecovirus and including the species of severe acute respiratory syndrome-related coronavirus. In specific embodiments, the disclosure encompasses treatment or prevention of infection of any virus in the species of severe acute respiratory syndrome-related coronavirus, including the strains severe acute respiratory syndrome coronavirus (SARS-CoV) and severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS, available on the World Wide Web at doi.org/10.1073/pnas.2004999117), cluster, or sub-cluster of the species of severe acute respiratory syndrome-related coronavirus, including at least SARS-CoV-2. In specific embodiments, the virus has a genome length between 29000 to 30000, between 29100 and 29900, between 29200 and 29900, between 29300 and 29900, between 29400 and 29900, between 29500 and 29900, between 29600 and 29900, between 29700 and 29900, between 29800 and 29900, or between 29780 and 29900 base pairs in length.

Examples of specific SARS-CoV-2 viruses include the following listed in the NCBI GenBank® Database, and these GenBank® Accession sequences are incorporated by reference herein in their entirety: (a) LC534419 and LC534418 and LC528233 and LC529905 (examples of different strains from Japan); (b) MT281577 and MT226610 and NC_045512 and MN996531 and MN908947 (examples of different strains from China); (c) MT281530 (Iran); (d) MT126808 (Brazil); (e) MT020781 (Finland); (f) MT093571 (Sweden); (g) MT263074 (Peru); (h) MT292582 and MT292581 and MT292580 and MT292579 (examples of different strains from Spain); (i) examples from the United States, such as MT276331 (TX); MT276330 (FL); MT276328 (OR) MT276327 (GA); MT276325 (WA); MT276324 (CA); MT276323 (RI); MT188341 (MN); and (j) MT276598 (Israel). In particular embodiments, the disclosure encompasses treatment or prevention of infection of any of these or similar viruses, including viruses whose genome has at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. In particular embodiments, the disclosure encompasses treatment or prevention of infection of any of these or similar viruses, including viruses whose genome has its entire sequence that is greater than 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. As one specific example, the present disclosure includes methods of treatment or prevention of infection of a virus having a genome sequence represented by GenBank® Accession No. NC 045512; origin Wuhan, China and any virus having a genome sequence with at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to a genome sequence represented by GenBank® Accession No. NC 045512.

SARS-CoV-2 proteins are described in detail in, for example, Yoshimoto F. K. (2020) The protein journal, 39(3), 198-216, incorporated herein by reference in its entirety.

### V. Antibodies, Antigen Binding Fragments, and Polypeptides

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. The term polypeptide also includes and antibody fragment described herein as well as antibody domains, such as HCDR1, HCDR2, HCDR3, LCDR1, LCDR2, LCDR3, HFRW1, HFRW2, HFRW3, FIFRW4, LFRW1, LFRW2, LFRW3, LFRW4, VH, VL, CH, or CL.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of an antibody, antigen binding fragment, protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The antibody, antigen binding fragment, polypeptides, proteins, or polynucleotides encoding such polypeptides or proteins of the disclosure may include 1, 2, 3, 4, 5, 6, 7, 8, 9, 11, 12, 13, 14, 15, 16, 17, 18, 19, 20, 21, 22, 23, 24, 25, 26, 27, 28, 29, 30, 31, 32, 33, 34, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NO:1-2812.

In some embodiments, the antibody, antigen binding fragment, protein, or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOS:1-2812.

In some embodiments, the antibody, antigen binding fragment, or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids or nucleic acids of SEQ ID NOs:1-2812.

In some embodiments, the antibody, antigen binding fragment, protein, or polypeptide may comprise at least, at most, or exactly 1, 2, 3, 4, 5, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 41, 42, 43, 44, 45, 46, 47, 48, 49, 50, 51, 52, 53, 54, 55, 56, 57, 58, 59, 60, 61, 62, 63, 64, 66, 67, 68, 69, 70, 71, 72, 73, 74, 75, 76, 77, 78, 79, 80, 81, 82, 83, 84, 85, 86, 87, 88, 89, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleic acids of SEQ ID NOS:1-2812 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS:1-2812.

In some aspects there is a nucleic acid molecule, antibody, antigen binding fragment, protein, or polypeptide starting at position 1, 2, 3, 4, 5, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 41, 42, 43, 44, 45, 46, 47, 48, 49, 50, 51, 52, 53, 54, 55, 56, 57, 58, 59, 60, 61, 62, 63, 64, 66, 67, 68, 69, 70, 71, 72, 73, 74, 75, 76, 77, 78, 79, 80, 81, 82, 83, 84, 85, 86, 87, 88, 89, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS:1-2812 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS:1-2812.

In some embodiments, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the heavy chain, light chain, VH, VL, HCDR1, HCDR2, HCDR3, LCDR1, LCDR2, LCDR3, HFRW1, HFRW2, HFRW3, HFRW4, LFRW1, LFRW2, LFRW3, or LFRW4 identified in Table 1 and SEQ ID NOS:1-1620 or 1825-2706 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some embodiments, a polypeptide (e.g., antibody, antibody fragment, Fab, etc.) of the disclosure comprises a CDR that is at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) in sequence to one of SEQ ID NOS:1-2804. In some embodiments, a polypeptide comprises 1, 2, and/or 3 CDRs from one of SEQ ID NOS:1-2804. The CDR may be one that has been determined by Kabat, IMGT, or Chothia. In further embodiments, a polypeptide may have CDRs that have 1, 2, and/or 3 amino acid changes (e.g., addition of 1 or 2 amino acids, deletions of 1 or 2 amino acids, substitution) with respect to these 1, 2, or 3 CDRs. In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

From amino to carboxy terminus the CDRs are CDR1, CDR2, and CDR3. In some embodiments, a polypeptide may have CDRs that have 1, 2, and/or 3 amino acid changes (e.g., addition of 1 or 2 amino acids, deletions of 1 or 2 amino acids, substitution) with respect to CDR1, CDR2, or CDR3. In some embodiments, the CDRs of SEQ ID NOS:1-2804 may further comprise 1, 2, 3, 4, 5, or 6 additional amino acids at the amino or carboxy terminus of the CDR, The additional amino acids may be from the heavy and/or light chain framework regions of SEQ ID NOS:44-76, that are shown as immediately adjacent to the CDRs. Accordingly, embodiments relate to polypeptides comprising an HCDR1 (i.e., CDR-H1), HCDR2 (i e., CDR-H2), HCDR3 (i.e., CDR-H3), LCDR1 (i.e., CDR-L1), LCDR2 (i. e., CDR-L2), and/or LCDR3 (i.e., CDR-L3) with at least or at most or exactly 1, 2, 3, 4, 5, 6 or 7 amino acids at the amino end of the CDR or at the carboxy end of the CDR, wherein the additional amino acids are the 1, 2, 3, 4, 5, 6, or 7 amino acids of Table 1 or SEQ ID NOS:1-2804 that are shown as immediately adjacent to the CDRs. Other embodiments relate to antibodies comprising one or more CDRs, wherein the CDR is a fragment of Table 1 or SEQ ID NOS:1-2804 and wherein the fragment lacks 1, 2, 3, 4, or 5 amino acids from the amino or carboxy end of the CDR. In some embodiments, the CDR may lack one, 2, 3, 4, 5, 6, or 7 amino acids from the carboxy end and may further comprise 1, 2, 3, 4, 5, 6, 7, or 8 amino acids from the framework region of the amino end of the CDR. In some embodiments, the CDR may lack one, 2, 3, 4, 5, 6, or 7 amino acids from the amino end and may further comprise 1, 2, 3, 4, 5, 6, 7, or 8 amino acids from the framework region of the carboxy end of the CDR. In further embodiments, an antibody may be alternatively or additionally humanized in regions outside the CDR(s) and/or variable region(s). In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

In other embodiments, a polypeptide or protein comprises 1, 2, 3, 4, 5, or 6 CDRs from either or both of the light and heavy variable regions of Table 1 or SEQ ID NOS:1-2804, and 1, 2, 3, 4, 5, or 6 CDRs may have 1, 2, and/or 3 amino acid changes with respect to these CDRs. In some embodiments, parts or all of the antibody sequence outside the variable region have been humanized. A protein may comprise one or more polypeptides. In some aspects, a protein may contain one or two polypeptides similar to a heavy chain polypeptide and/or 1 or 2 polypeptides similar to a light chain polypeptide.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about mg and about 10 mg of total polypeptide, peptide, and/or protein per ml. The concentration of protein in a composition can be about, at least about or at most about 0.001, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 6.0, 6.5, 7.0, 7.5, 8.0, 8.5, 9.0, 9.5, 10.0 mg/ml or more (or any range derivable therein).

### VI. Sequences

Polypeptide, antibody, and antigen binding fragment embodiments are shown below in the following tables.

TABLE 1
Antibody and antigen binding embodiments

| | | | SEQ ID |
|---|---|---|---|
| Clone | Description | Sequence | NO: |
| S20-15 | Heavy Chain | QVQLQESGPGLVRPSETLSLTCTVSGGSISSHYWSWIRQPPGKGLEWI | 1 |
| (Spike/ | | GYIYYSGSTNYNPSLKSRVTISVDTSKNQFSLKLISVTAADTAVYYCA | |
| RBD) | | RAGGVFGVVLDFDHWGRGTLVTVSSASTKGPSVFPLAPSSKSTSGGT | |
| | | AALGCLVKDYFPEPVTVSWNSGALTSGVHTFPAVLQSSG | |
| | Heavy Chain | QVQLQESGPGLVRPSETLSLTCTVSGGSISSHYWSWIRQPPGKGLEWI | 2 |
| | Variable | GYIYYSGSTNYNPSLKSRVTISVDTSKNQFSLKLISVTAADTAVYYCA | |
| | Region | RAGGVFGVVLDFDHWGRGTLVTVSS | |
| | HCDR1 | SHYWS | 3 |
| | HCDR2 | YIYYSGSTNYNPSLKS | 4 |
| | HCDR3 | AGGVFGVVLDFDH | 5 |
| | HFRW1 | QVQLQESGPGLVRPSETLSLTCTVSGGSIS | 6 |
| | HFRW2 | WIRQPPGKGLEWIG | 7 |
| | HFRW3 | RVTISVDTSKNQFSLKLISVTAADTAVYYCAR | 8 |
| | HFRW4 | WGRGTLVTVSS | 9 |
| | Light Chain | SYVLTQPPSVSVAPGQTARITCGGNNIGSKSVHWYQQKPGQAPVLVV | 10 |
| | | YDDSDRPSGIPERFSGSNSGNTATLTISRVEAGDEADYYCQVWDSSSE | |
| | | HYVFGTGTKVTVLGQPKANPTVTLFPPSSEELQANKATLVCLISDFYP | |
| | | GAVTVAWKADGSPVKAGVETTKPSKQSNNKYAASS | |
| | Light Chain | SYVLTQPPSVSVAPGQTARITCGGNNIGSKSVHWYQQKPGQAPVLVV | 11 |
| | Variable | YDDSDRPSGIPERFSGSNSGNTATLTISRVEAGDEADYYCQVWDSSSE | |
| | Region | HYVFGTGTKVTVL | |
| | LCDR1 | GGNNIGSKSVH | 12 |
| | LCDR2 | DDSDRPS | 13 |
| | LCDR3 | QVWDSSSEHYV | 14 |
| | LFRW1 | SYVLTQPPSVSVAPGQTARITC | 15 |
| | LFRW2 | WYQQKPGQAPVLVVY | 16 |
| | LFRW3 | GIPERFSGSNSGNTATLTISRVEAGDEADYYC | 17 |
| | LFRW4 | FGTGTKVTVL | 18 |
| S20-22 | Heavy Chain | QVQLQESGPGLVKPSETLSLTCTVSGGSISSFYWGWIRQPAGKGLEWI | 19 |
| (NP) | | GRFHTSGSTNYNPSFKSRVTMSVDTSKNQFSLKLTSVTAADTAVYYC | |
| | | ASGRGSSWYVGWFFDLWGRGTLVTVSSASTKGPSVFPLAPSSKSTSG | |
| | | GTAALGCLVKDYFPEPVTVSWNSGALTSGVHTFPAVLQSSG | |
| | Heavy Chain | QVQLQESGPGLVKPSETLSLTCTVSGGSISSFYWGWIRQPAGKGLEWI | 20 |
| | Variable | GRFHTSGSTNYNPSFKSRVTMSVDTSKNQFSLKLTSVTAADTAVYYC | |
| | Region | ASGRGSSWYVGWFFDLWGRGTLVTVSS | |
| | HCDR1 | SFYWG | 21 |
| | HCDR2 | RFHTSGSTNYNPSFKS | 22 |
| | HCDR3 | GRGSSWYVGWFFDL | 23 |
| | HFRW1 | QVQLQESGPGLVKPSETLSLTCTVSGGSIS | 24 |
| | HFRW2 | WIRQPAGKGLEWIG | 25 |
| | HFRW3 | RVTMSVDTSKNQFSLKLTSVTAADTAVYYCAS | 26 |
| | HFRW4 | WGRGTLVTVSS | 27 |
| | Light Chain | DIVMTQSPDSLAVSLGERATINCKSSQTVLYSSNNKNYLAWYQQKPG | 28 |
| | | QPPKLLIYWASTRESGVPDRFSGSGSGTDFTLTISSLQAGDVAVYYCQ | |
| | | QYYNTPDTFGGGTKVEINRTVAAPSVFIFPPSDEQLKSGTASVVCLLN | |
| | | NFYPREAKVQWKVDN | |
| | Light Chain | DIVMTQSPDSLAVSLGERATINCKSSQTVLYSSNNKNYLAWYQQKPG | 29 |
| | Variable | QPPKLLIYWASTRESGVPDRFSGSGSGTDFTLTISSLQAGDVAVYYCQ | |
| | Region | QYYNTPDTFGGGTKVEI | |
| | LCDR1 | KSSQTVLYSSNNKNYLA | 30 |
| | LCDR2 | WASTRES | 31 |
| | LCDR3 | QQYYNTPDT | 32 |
| | LFRW1 | DIVMTQSPDSLAVSLGERATINC | 33 |
| | LFRW2 | WYQQKPGQPPKLLIY | 34 |
| | LFRW3 | GVPDRFSGSGSGTDFTLTISSLQAGDVAVYYC | 35 |
| | LFRW4 | FGGGTKVEI | 36 |
| S20-31 | Heavy Chain | QVQLIQSGAEVKKPGASVKVSCTASGYSLNELPIQWVRQAPGKGLEW | 37 |
| (NP) | | MGEFDPEDGETIYAEKFQGRVTLTEETSTNTAYMELSSLKSEDTAAYF | |
| | | CSTGSTIGVVIYAFAIWGQGTMVTVSSASTKGPSVFPLAPCSRSTSEST | |
| | | AALGCLVKDYFPEPVTVSWNSGALTSGVHTFPAVLQSSG | |
| | Heavy Chain | QVQLIQSGAEVKKPGASVKVSCTASGYSLNELPIQWVRQAPGKGLEW | 38 |
| | Variable | MGEFDPEDGETIYAEKFQGRVTLTEETSTNTAYMELSSLKSEDTAAYF | |
| | Region | CSTGSTIGVVIYAFAIWGQGTMVTVSS | |
| | HCDR1 | ELPIQ | 39 |
| | HCDR2 | EFDPEDGETIYAEKFQG | 40 |
| | HCDR3 | GSTIGVVIYAFAI | 41 |
| | HFRW1 | QVQLIQSGAEVKKPGASVKVSCTASGYSLN | 42 |
| | HFRW2 | WVRQAPGKGLEWMG | 43 |
| | HFRW3 | RVTLTEETSTNTAYMELSSLKSEDTAAYFCST | 44 |
| | HFRW4 | WGQGTMVTVSS | 45 |
| | Light Chain | EIVLTQSPGTLSLSPGERATLSCRASQDITNNFLAWYQQKAGQAPKLFI | 46 |
| | | YGASRRAPGIPHRFSGSGSGTDFTLTISSLEPEDFAVYYCQQYGPSPTF | |
| | | GQGTKVEIKRTVAAPSVFIFPPSDEQLKSGTASVVCLLNNFYPREAKV | |
| | | QWKVDNALQSGNSQESVTEQDSKDSTYSLSSTLTLSKADYE | |
| | Light Chain | EIVLTQSPGTLSLSPGERATLSCRASQDITNNFLAWYQQKAGQAPKLFI | 47 |
| | Variable | YGASRRAPGIPHRFSGSGSGTDFTLTISSLEPEDFAVYYCQQYGPSPTF | |
| | Region | GQGTKVEIK | |
| | LCDR1 | RASQDITNNFLA | 48 |
| | LCDR2 | GASRRAP | 49 |
| | LCDR3 | QQYGPSPT | 50 |
| | LFRW1 | EIVLTQSPGTLSLSPGERATLSC | 51 |
| | LFRW2 | WYQQKAGQAPKLFIY | 52 |
| | LFRW3 | GIPHRFSGSGSGTDFTLTISSLEPEDFAVYYC | 53 |
| | LFRW4 | FGQGTKVEIK | 54 |
| S20-40 | Heavy Chain | QVQLQESGPGLVKPSETLSLTCTVSGGSISSYYWSWIRQPAGKGLEWI | 55 |
| (NP) | | GRIYTSGSTNYNPSLKSRVTMSVDTSKNQFSLKLSSVTAADTAVYYC | |
| | | ARGGSGWRFDYWGQGTLVTVSSGSASAPTLFPLVSCENSPSDTSSV | |
| | Heavy Chain | QVQLQESGPGLVKPSETLSLTCTVSGGSISSYYWSWIRQPAGKGLEWI | 56 |
| | Variable | GRIYTSGSTNYNPSLKSRVTMSVDTSKNQFSLKLSSVTAADTAVYYC | |
| | Region | ARGGSGWRFDYWGQGTLVTVSS | |
| | HCDR1 | SYYWS | 57 |
| | HCDR2 | RIYTSGSTNYNPSLKS | 58 |
| | HCDR3 | GGSGWRFDY | 59 |
| | HFRW1 | QVQLQESGPGLVKPSETLSLTCTVSGGSIS | 60 |
| | HFRW2 | WIRQPAGKGLEWIG | 61 |
| | HFRW3 | RVTMSVDTSKNQFSLKLSSVTAADTAVYYCAR | 62 |
| | HFRW4 | WGQGTLVTVSS | 63 |
| | Light Chain | QSALTQPASVSGSPGQSITISCTGTSSDVGGYNYVSWYQQHPGKAPKL | 64 |
| | | MIYDVSNRPSGVSNRFSGSKSGNTASLTISGLQAEDEADYYCSSYTSS | |
| | | STLGVFGGGTKLTVLGQPKAAPSVTLFPPSSEELQANKATLVCLISDF | |
| | | YPGAVTVAWKADSSPVKAGVETTTPSKQSNNKYAASS | |
| | Light Chain | QSALTQPASVSGSPGQSITISCTGTSSDVGGYNYVSWYQQHPGKAPKL | 65 |
| | Variable | MIYDVSNRPSGVSNRFSGSKSGNTASLTISGLQAEDEADYYCSSYTSS | |
| | Region | STLGVFGGGTKLTVL | |
| | LCDR1 | TGTSSDVGGYNYVS | 66 |
| | LCDR2 | DVSNRPS | 67 |
| | LCDR3 | SSYTSSSTLGV | 68 |
| | LFRW1 | QSALTQPASVSGSPGQSITISC | 69 |
| | LFRW2 | WYQQHPGKAPKLMIY | 70 |
| | LFRW3 | GVSNRFSGSKSGNTASLTISGLQAEDEADYYC | 71 |
| | LFRW4 | FGGGTKLTVL | 72 |
| S20-58 | Heavy Chain | QVQLQESGPGLVKPSQTLSLTCTVSGGSINSGDYYWSWIRQPPGKGLE | 73 |
| (Spike/ | | WIGYIYFSGSTYYNPSLKSRVTISLDRSKNQFSLKLSSVTAADTAVYY | |
| RBD) | | CAREESMITLGGVIVDWGQGTLVTVSSASTKGPSVFPLAPSSKSTSGG | |
| | | TAALGCLVKDYFPEPVTVSWNSGALTSGVHTFPAVLQSSG | |
| | Heavy Chain | QVQLQESGPGLVKPSQTLSLTCTVSGGSINSGDYYWSWIRQPPGKGLE | 74 |
| | Variable | WIGYIYFSGSTYYNPSLKSRVTISLDRSKNQFSLKLSSVTAADTAVYY | |
| | Region | CAREESMITLGGVIVDWGQGTLVTVSS | |
| | HCDR1 | SGDYYWS | 75 |
| | HCDR2 | YIYFSGSTYYNPSLKS | 76 |
| | HCDR3 | EESMITLGGVIVD | 77 |
| | HFRW1 | QVQLQESGPGLVKPSQTLSLTCTVSGGSIN | 78 |
| | HFRW2 | WIRQPPGKGLEWIG | 79 |
| | HFRW3 | RVTISLDRSKNQFSLKLSSVTAADTAVYYCAR | 80 |
| | HFRW4 | WGQGTLVTVSS | 81 |
| | Light Chain | DIVMTQTPLSSPVTLGQPASISCRSSQSLVHSDGDTYLSWLQQRPGQP | 82 |
| | | PRLLIYKISNRFSGVPDRFSGSGAGTDFTLKISRVEAEDVGVYYCMQA | |
| | | TQFPLTFGGGTKVEIKRTVAAPSVFIFPPSDEQLKSGTASVVCLLNNFY | |
| | | PREAKVQWKVDNALQSGNSQESVTEQDSKDSTYSLSSTLTLSKADYE | |
| | Light Chain | DIVMTQTPLSSPVTLGQPASISCRSSQSLVHSDGDTYLSWLQQRPGQP | 83 |
| | Variable | PRLLIYKISNRFSGVPDRFSGSGAGTDFTLKISRVEAEDVGVYYCMQA | |
| | Region | TQFPLTFGGGTKVEIK | |
| | LCDR1 | RSSQSLVHSDGDTYLS | 84 |
| | LCDR2 | KISNRFS | 85 |
| | LCDR3 | MQATQFPLT | 86 |
| | LFRW1 | DIVMTQTPLSSPVTLGQPASISC | 87 |
| | LFRW2 | WLQQRPGQPPRLLIY | 88 |
| | LFRW3 | GVPDRFSGSGAGTDFTLKISRVEAEDVGVYYC | 89 |
| | LFRW4 | FGGGTKVEIK | 90 |
| S20-74 | Heavy Chain | QVQLQESGPGLVKPSETLSLTCTVSGGSISSHYWSWIRQPPGKGLEQI | 91 |
| (Spike/ | | GYMYYSGSTNYNPSLKSRVIISVDTSKNQFSLKLSSVTAADTAVYYC | |
| RBD) | | AGRDQLLYGADGFDIWGQGTMVTVSSASTKGPSVFPLAPSSKSTSGG | |
| | | TAALGCLVKDYFPEPVTVSWNSGALTSGVHTFPAVLQSSG | |
| | Heavy Chain | QVQLQESGPGLVKPSETLSLTCTVSGGSISSHYWSWIRQPPGKGLEQI | 92 |
| | Variable | GYMYYSGSTNYNPSLKSRVIISVDTSKNQFSLKLSSVTAADTAVYYC | |
| | Region | AGRDQLLYGADGFDIWGQGTMVTVSS | |
| | HCDR1 | SHYWS | 93 |
| | HCDR2 | YMYYSGSTNYNPSLKS | 94 |
| | HCDR3 | RDQLLYGADGFDI | 95 |
| | HFRW1 | QVQLQESGPGLVKPSETLSLTCTVSGGSIS | 96 |
| | HFRW2 | WIRQPPGKGLEQIG | 97 |
| | HFRW3 | RVIISVDTSKNQFSLKLSSVTAADTAVYYCAG | 98 |
| | HFRW4 | WGQGTMVTVSS | 99 |
| | Light Chain | QSALTQPPSASGSPGQSVTISCTGTSSDVGGYNYVSWYQQHPGKAPK | 100 |
| | | LMIYEVSKRPSGVPDRYSGSKSGNTASLTVSGLQAEDEADYYCSSYA | |
| | | GSSNHVIFGGGTKLTVLGQPKAAPSVTLFPPSSEELQANKATLVCLISD | |
| | | FYPGAVTVAWKADSSPVKAGVETTTPSKQSNNKYAASS | |
| | Light Chain | QSALTQPPSASGSPGQSVTISCTGTSSDVGGYNYVSWYQQHPGKAPK | 101 |
| | Variable | LMIYEVSKRPSGVPDRYSGSKSGNTASLTVSGLQAEDEADYYCSSYA | |
| | Region | GSSNHVIFGGGTKLTVL | |
| | LCDR1 | TGTSSDVGGYNYVS | 102 |
| | LCDR2 | EVSKRPS | 103 |
| | LCDR3 | SSYAGSSNHVI | 104 |
| | LFRW1 | QSALTQPPSASGSPGQSVTISC | 105 |
| | LFRW2 | WYQQHPGKAPKLMIY | 106 |
| | LFRW3 | GVPDRYSGSKSGNTASLTVSGLQAEDEADYYC | 107 |
| | LFRW4 | FGGGTKLTVL | 108 |
| S20-86 | Heavy Chain | EVQLVESGGGLVQPGRSLRLSCAASGFTFGDYAMYWVRQPPGKGLE | 109 |
| (Spike) | | WVSGISWNRGTIGYADSVKGRFTISRDNAKNSLYLQMNSLTPEDTAL | |
| | | YYCAKDMLPASRFFYYMDVWGKGTTVIVSSASTKGPSVFPLAPSSKS | |
| | | TSGGTAALGCLVKDYFPEPVTVSWNSGALTSGVHTFPAVLQSSG | |
| | Heavy Chain | EVQLVESGGGLVQPGRSLRLSCAASGFTFGDYAMYWVRQPPGKGLE | 110 |
| | Variable | WVSGISWNRGTIGYADSVKGRFTISRDNAKNSLYLQMNSLTPEDTAL | |
| | Region | YYCAKDMLPASRFFYYMDVWGKGTTVIVSS | |
| | HCDR1 | DYAMY | 111 |
| | HCDR2 | GISWNRGTIGYADSVKG | 112 |
| | HCDR3 | DMLPASRFFYYMDV | 113 |
| | HFRW1 | EVQLVESGGGLVQPGRSLRLSCAASGFTFG | 114 |
| | HFRW2 | WVRQPPGKGLEWVS | 115 |
| | HFRW3 | RFTISRDNAKNSLYLQMNSLTPEDTALYYCAK | 116 |
| | HFRW4 | WGKGTTVIVSS | 117 |
| | Light Chain | QSALTQPASVSGSPGQSITISCTGTSSDVGGYNYVSWYQQHPGKAPKL | 118 |
| | | MIYDVSNRPSGVSNRFSGSKSGNTASLTISGLQAEDEADYYCSSYTSS | |
| | | STLGVFGTGTKVTVLGQPKANPTVTLFPPSSEELQANKATLVCLISDF | |
| | | YPGAVTVAWKADGSPVKAGVETTKPSKQSNNKYAASS | |
| | Light Chain | QSALTQPASVSGSPGQSITISCTGTSSDVGGYNYVSWYQQHPGKAPKL | 119 |
| | Variable | MIYDVSNRPSGVSNRFSGSKSGNTASLTISGLQAEDEADYYCSSYTSS | |
| | Region | STLGVFGTGTKVTVL | |
| | LCDR1 | TGTSSDVGGYNYVS | 120 |
| | LCDR2 | DVSNRPS | 121 |
| | LCDR3 | SSYTSSSTLGV | 122 |
| | LFRW1 | QSALTQPASVSGSPGQSITISC | 123 |
| | LFRW2 | WYQQHPGKAPKLMIY | 124 |
| | LFRW3 | GVSNRFSGSKSGNTASLTISGLQAEDEADYYC | 125 |
| | LFRW4 | FGTGTKVTVL | 126 |
| S24-68 | Heavy Chain | QVQLQESGPGLVKPSETLSLTCTVSGGSITSYYWSWIRQPPGKGLEWI | 127 |
| (ORF8) | | EYIHYSGSTNYNPSLKSRVTISVDTSKNQFSLKLSSVTAADTAVYYCA | |
| | | RLLKYSRGGCYFDHWGQGTLVTVSSASTKGPSVFPLAPSSKSTSGGT | |
| | | AALGCLVKDYFPEPVTVSWNSGALTSGVHTFPAVLQSSG | |
| | Heavy Chain | QVQLQESGPGLVKPSETLSLTCTVSGGSITSYYWSWIRQPPGKGLEWI | 128 |
| | Variable | EYIHYSGSTNYNPSLKSRVTISVDTSKNQFSLKLSSVTAADTAVYYCA | |
| | Region | RLLKYSRGGCYFDHWGQGTLVTVSS | |
| | HCDR1 | SYYWS | 129 |
| | HCDR2 | YIHYSGSTNYNPSLKS | 130 |
| | HCDR3 | LLKYSRGGCYFDH | 131 |
| | HFRW1 | QVQLQESGPGLVKPSETLSLTCTVSGGSIT | 132 |
| | HFRW2 | WIRQPPGKGLEWIE | 133 |
| | HFRW3 | RVTISVDTSKNQFSLKLSSVTAADTAVYYCAR | 134 |
| | HFRW4 | WGQGTLVTVSS | 135 |
| | Light Chain | QSVLTQPPSASGTPGQRVTISCSGSSSNIGGNPVNWYQQLPGTAPKLLI | 136 |
| | | YSNNQRPSGVPDRFSGSKSGTSASLAISGLQSEDEADYYCAAWDDSL | |
| | | KGPVFGGGTKLTVLGQPKAAPSVTLFPPSSEELQANKATLVCLISDFY | |
| | | PGAVTVAWKADSSPVKAGVETTTPSKQSNNKYAASSYLSLTPEQWK | |
| | | SH | |
| | Light Chain | QSVLTQPPSASGTPGQRVTISCSGSSSNIGGNPVNWYQQLPGTAPKLLI | 137 |
| | Variable | YSNNQRPSGVPDRFSGSKSGTSASLAISGLQSEDEADYYCAAWDDSL | |
| | Region | KGPVFGGGTKLTVL | |
| | LCDR1 | SGSSSNIGGNPVN | 138 |
| | LCDR2 | SNNQRPS | 139 |
| | LCDR3 | AAWDDSLKGPV | 140 |
| | LFRW1 | QSVLTQPPSASGTPGQRVTISC | 141 |
| | LFRW2 | WYQQLPGTAPKLLIY | 142 |
| | LFRW3 | GVPDRFSGSKSGTSASLAISGLQSEDEADYYC | 143 |
| | LFRW4 | FGGGTKLTVL | 144 |
| S24-105 | Heavy Chain | EVQLVESGGGLVQPGGSLRLSCAASGFTLSSYSMNWVRQAPGKGLE | 145 |
| (ORF8) | | WVSYISSSSSTIYYADSVKGRFTISKDNAKNSLYLQMNSLRAEDTAVY | |
| | | YCAVGRGYFVYWGQGTLVTVSSASTKGPSVFPLAPSSKSTSGGTAAL | |
| | | GCLVKDYFPEPVTVSWNSGALTSGVHTFPAVLQSSG | |
| | Heavy Chain | EVQLVESGGGLVQPGGSLRLSCAASGFTLSSYSMNWVRQAPGKGLE | 146 |
| | Variable | WVSYISSSSSTIYYADSVKGRFTISKDNAKNSLYLQMNSLRAEDTAVY | |
| | Region | YCAVGRGYFVYWGQGTLVTVSS | |
| | HCDR1 | SYSMN | 147 |
| | HCDR2 | YISSSSSTIYYADSVKG | 148 |
| | HCDR3 | GRGYFVY | 149 |
| | HFRW1 | EVQLVESGGGLVQPGGSLRLSCAASGFTLS | 150 |
| | HFRW2 | WVRQAPGKGLEWVS | 151 |
| | HFRW3 | RFTISKDNAKNSLYLQMNSLRAEDTAVYYCAV | 152 |
| | HFRW4 | WGQGTLVTVSS | 153 |
| | Light Chain | EIVLTQSPGTLSLSPGERATLSCRASQSVSSGYLAWYQQKPGQAPRLLI | 154 |
| | | FGASSRATGIPDRFSGSGSGTDFTLTINRLEPEDFAVYYCQQYGSSRTF | |
| | | GQGTKVEIKRTVAAPSVFIFPPSDEQLKSGTASVVCLLNNFYPREAKV | |
| | | QWKVDNALQSGNSQESVTEQDSKDSTYSLSSTLTLSKADYE | |
| | Light Chain | EIVLTQSPGTLSLSPGERATLSCRASQSVSSGYLAWYQQKPGQAPRLLI | 155 |
| | Variable | FGASSRATGIPDRFSGSGSGTDFTLTINRLEPEDFAVYYCQQYGSSRTF | |
| | Region | GQGTKVEIK | |
| | LCDR1 | RASQSVSSGYLA | 156 |
| | LCDR2 | GASSRAT | 157 |
| | LCDR3 | QQYGSSRT | 158 |
| | LFRW1 | EIVLTQSPGTLSLSPGERATLSC | 159 |
| | LFRW2 | WYQQKPGQAPRLLIF | 160 |
| | LFRW3 | GIPDRFSGSGSGTDFTLTINRLEPEDFAVYYC | 161 |
| | LFRW4 | FGQGTKVEIK | 162 |
| S24-178 | Heavy Chain | QVQLVESGGGVVQPGRSLRLSCAASGFTFSSYGMHWVRQAPGKGLE | 163 |
| (NP) | | WVAVIWYDGSNKYYADSVKGRFTISRDNSKNTLYLQMNSLRAEDTA | |
| | | VYYCARIEGYSYGDVRVYYYYGMDVWGQGTTVTVSSASTKGPSVFP | |
| | | LAPSSKSTSGGTAALGCLVKDYFPEPVTVSWNSGALTSGVHTFPAVL | |
| | | QSSG | |
| | Heavy Chain | QVQLVESGGGVVQPGRSLRLSCAASGFTFSSYGMHWVRQAPGKGLE | 164 |
| | Variable | WVAVIWYDGSNKYYADSVKGRFTISRDNSKNTLYLQMNSLRAEDTA | |
| | Region | VYYCARIEGYSYGDVRVYYYYGMDVWGQGTTVTVSS | |
| | HCDR1 | SYGMH | 165 |
| | HCDR2 | VIWYDGSNKYYADSVKG | 166 |
| | HCDR3 | IEGYSYGDVRVYYYYGMDV | 167 |
| | HFRW1 | QVQLVESGGGVVQPGRSLRLSCAASGFTFS | 168 |
| | HFRW2 | WVRQAPGKGLEWVA | 169 |
| | HFRW3 | RFTISRDNSKNTLYLQMNSLRAEDTAVYYCAR | 170 |
| | HFRW4 | WGQGTTVTVSS | 171 |
| | Light Chain | QSALTQPASVSGSPGQSITISCTGTTSDVGGYDYVSWYQQHPGKAPKL | 172 |
| | | ILSEVSNRPSGVSNRFSGSKSGNTASLTISGLQAEDEADYYCSSYPSSS | |
| | | TLVFGTGTKVTVLGQPKANPTVTLFPPSSEELQANKATLVCLISDFYP | |
| | | GAVTVAWKADGSPVKAGVETTTPSKQSNNKYAASS | |
| | Light Chain | QSALTQPASVSGSPGQSITISCTGTTSDVGGYDYVSWYQQHPGKAPKL | 173 |
| | Variable | ILSEVSNRPSGVSNRFSGSKSGNTASLTISGLQAEDEADYYCSSYPSSS | |
| | Region | TLVFGTGTKVTVL | |
| | LCDR1 | TGTTSDVGGYDYVS | 174 |
| | LCDR2 | EVSNRPS | 175 |
| | LCDR3 | SSYPSSSTLV | 176 |
| | LFRW1 | QSALTQPASVSGSPGQSITISC | 177 |
| | LFRW2 | WYQQHPGKAPKLILS | 178 |
| | LFRW3 | GVSNRFSGSKSGNTASLTISGLQAEDEADYYC | 179 |
| | LFRW4 | FGTGTKVTVL | 180 |
| S24-188 | Heavy Chain | QVHLVQSGAEVKKPGSSVKVSCKASGGTFSSCAISWVRQAPGQGLE | 181 |
| (NP) | | WMGRIIPILGIANYAQKFQGRVTITADKSTSTAYMELSSLRSEDTAVY | |
| | | YCARGWEFGSGSYYRTDYYYYAMDVWGQGTTVTVSSASTKGPSVF | |
| | | PLAPCSRSTSGGTAALGCLVKDYFPEPVTVSWNSGALTSGVHTFPAV | |
| | | LQSSG | |
| | Heavy Chain | QVHLVQSGAEVKKPGSSVKVSCKASGGTFSSCAISWVRQAPGQGLE | 182 |
| | Variable | WMGRIIPILGIANYAQKFQGRVTITADKSTSTAYMELSSLRSEDTAVY | |
| | Region | YCARGWEFGSGSYYRTDYYYYAMDVWGQGTTVTVSS | |
| | HCDR1 | SCAIS | 183 |
| | HCDR2 | RIIPILGIANYAQKFQG | 184 |
| | HCDR3 | GWEFGSGSYYRTDYYYYAMDV | 185 |
| | HFRW1 | QVHLVQSGAEVKKPGSSVKVSCKASGGTFS | 186 |
| | HFRW2 | WVRQAPGQGLEWMG | 187 |
| | HFRW3 | RVTITADKSTSTAYMELSSLRSEDTAVYYCAR | 188 |
| | HFRW4 | WGQGTTVTVSS | 189 |
| | Light Chain | QSALTQPASVSGSPGQSITISCTGTSSDVGGYNYVSWYQQHPGKAPKL | 190 |
| | | MIYEVTNRPSGVSNRFSGSRSGNTASLTISGLQAEDEADYYCSSYTSSS | |
| | | LYVFGTGTKVAVLGQPKANPTVTLFPPSSEELQANKATLVCLISDFYP | |
| | | GAVTVAWKADSSPVKAGVETTKPSKQSNNKYAASS | |
| | Light Chain | QSALTQPASVSGSPGQSITISCTGTSSDVGGYNYVSWYQQHPGKAPKL | 191 |
| | Variable | MIYEVTNRPSGVSNRFSGSRSGNTASLTISGLQAEDEADYYCSSYTSSS | |
| | Region | LYVFGTGTKVAVL | |
| | LCDR1 | TGTSSDVGGYNYVS | 192 |
| | LCDR2 | EVTNRPS | 193 |
| | LCDR3 | SSYTSSSLYV | 194 |
| | LFRW1 | QSALTQPASVSGSPGQSITISC | 195 |
| | LFRW2 | WYQQHPGKAPKLMIY | 196 |
| | LFRW3 | GVSNRFSGSRSGNTASLTISGLQAEDEADYYC | 197 |
| | LFRW4 | FGTGTKVAVL | 198 |
| S24-202 | Heavy Chain | EVQLVQSGAEVKKPGESLRISCKGSGYSFSSYWISWVRQMPGKGLEW | |
| (NP) | | MGRIDPSDSNTNYSPSFQGHVTISADKSISTAYLQWSSLKASDTAMYY | |
| | | CARLSVRVWFGELPHYGMDVWGQGTTVTVSSASTKGPSVFPLAPSS | |
| | | KSTSGGTAALGCLVKDYFPEPVTVSWNSGALTSGVHTFPAVLQSSG | 199 |
| | Heavy Chain | EVQLVQSGAEVKKPGESLRISCKGSGYSFSSYWISWVRQMPGKGLEW | 200 |
| | Variable | MGRIDPSDSNTNYSPSFQGHVTISADKSISTAYLQWSSLKASDTAMYY | |
| | Region | CARLSVRVWFGELPHYGMDVWGQGTTVTVSS | |
| | HCDR1 | SYWIS | 201 |
| | HCDR2 | RIDPSDSNTNYSPSFQG | 202 |
| | HCDR3 | LSVRVWFGELPHYGMDV | 203 |
| | HFRW1 | EVQLVQSGAEVKKPGESLRISCKGSGYSFS | 204 |
| | HFRW2 | WVRQMPGKGLEWMG | 205 |
| | HFRW3 | HVTISADKSISTAYLQWSSLKASDTAMYYCAR | 206 |
| | HFRW4 | WGQGTTVTVSS | 207 |
| | Light Chain | EIVLTQSPATLSLSPGERATLSCRASQSVSSYLAWYQQKPGQAPRLLIY | 208 |
| | | DASNRASGIPARFSGSGSGTDFTLTISSLEPEDFAVYYCQQRRNWPLTF | |
| | | GGGTKVETKRTVAAPSVFIFPPSDEQLKSGTASVVCLLNNFYPREAKV | |
| | | QWKVDN | |
| | Light Chain | EIVLTQSPATLSLSPGERATLSCRASQSVSSYLAWYQQKPGQAPRLLIY | 209 |
| | Variable | DASNRASGIPARFSGSGSGTDFTLTISSLEPEDFAVYYCQQRRNWPLTF | |
| | Region | GGGTKVETK | |
| | LCDR1 | RASQSVSSYLA | 210 |
| | LCDR2 | DASNRAS | 211 |
| | LCDR3 | QQRRNWPLT | 212 |
| | LFRW1 | EIVLTQSPATLSLSPGERATLSC | 213 |
| | LFRW2 | WYQQKPGQAPRLLIY | 214 |
| | LFRW3 | GIPARFSGSGSGTDFTLTISSLEPEDFAVYYC | 215 |
| | LFRW4 | FGGGTKVETK | 216 |
| S24-278 | Heavy Chain | QVQLVQSGAEVKKPGASVKVSCKASGYTFTGYYMHWVRQAPGQGL | 217 |
| (ORF8) | | EWMGWINPNSGDTNYAQKFQGWVTMTRDTSLSTAYMELSRLKSDD | |
| | | TAVYYCARVGVGEYSGRHYYYYGMDVWGQGTTVTVSSASTKGPSV | |
| | | FPLAPSSKSTSGGTAALGCLVKDYFPEPVTVSWNSGALTSGVHTFPAV | |
| | | LQSSG | |
| | Heavy Chain | QVQLVQSGAEVKKPGASVKVSCKASGYTFTGYYMHWVRQAPGQGL | 218 |
| | Variable | EWMGWINPNSGDTNYAQKFQGWVTMTRDTSLSTAYMELSRLKSDD | |
| | Region | TAVYYCARVGVGEYSGRHYYYYGMDVWGQGTTVTVSS | |
| | HCDR1 | GYYMH | 219 |
| | HCDR2 | WINPNSGDTNYAQKFQG | 220 |
| | HCDR3 | VGVGEYSGRHYYYYGMDV | 221 |
| | HFRW1 | QVQLVQSGAEVKKPGASVKVSCKASGYTFT | 222 |
| | HFRW2 | WVRQAPGQGLEWMG | 223 |
| | HFRW3 | WVTMTRDTSLSTAYMELSRLKSDDTAVYYCAR | 224 |
| | HFRW4 | WGQGTTVTVSS | 225 |
| | Light Chain | EIVLTQSPGTLSLSPGERATLSCRASQSISSSYLAWYQQKPGQAPRLLI | 226 |
| | | YGASSRATGIPDRFSGSGSGTDFTLTISRLEPEDFAVYYCQQYGSSLTF | |
| | | GGGTKVEIKRTVAAPSVFIFPPSDEQLKSGTASVVCLLNNFYPREAKV | |
| | | QWKVDN | |
| | Light Chain | EIVLTQSPGTLSLSPGERATLSCRASQSISSSYLAWYQQKPGQAPRLLI | 227 |
| | Variable | YGASSRATGIPDRFSGSGSGTDFTLTISRLEPEDFAVYYCQQYGSSLTF | |
| | Region | GGGTKVEIK | |
| | LCDR1 | RASQSISSSYLA | 228 |
| | LCDR2 | GASSRAT | 229 |
| | LCDR3 | QQYGSSLT | 230 |
| | LFRW1 | EIVLTQSPGTLSLSPGERATLSC | 231 |
| | LFRW2 | WYQQKPGQAPRLLIY | 232 |
| | LFRW3 | GIPDRFSGSGSGTDFTLTISRLEPEDFAVYYC | 233 |
| | LFRW4 | FGGGTKVEIK | 234 |
| S24-339 | Heavy Chain | EVQLVESGGGLVQPGRSLRLSCTASGFTFGDYAMSWFRQAPGKGLE | 235 |
| (Spike/ | | WVGFIRSKAYGGTTQHAASVKGRFTISRDDSKSIAYLQMNSLKTEDT | |
| RBD) | | AVYHCARDGYDCSGGRCYSHIFDYWGQGTLVTVSSGESSPPPL*VHL | |
| | | GRLSLPGSQGQSLV | |
| | Heavy Chain | EVQLVESGGGLVQPGRSLRLSCTASGFTFGDYAMSWFRQAPGKGLE | 236 |
| | Variable | WVGFIRSKAYGGTTQHAASVKGRFTISRDDSKSIAYLQMNSLKTEDT | |
| | Region | AVYHCARDGYDCSGGRCYSHIFDYWGQGTLVTVSS | |
| | HCDR1 | DYAMS | 237 |
| | HCDR2 | FIRSKAYGGTTQHAASVKG | 238 |
| | HCDR3 | DGYDCSGGRCYSHIFDY | 239 |
| | HFRW1 | EVQLVESGGGLVQPGRSLRLSCTASGFTFG | 240 |
| | HFRW2 | WFRQAPGKGLEWVG | 241 |
| | HFRW3 | RFTISRDDSKSIAYLQMNSLKTEDTAVYHCAR | 242 |
| | HFRW4 | WGQGTLVTVSS | 243 |
| | Light Chain | EIVMTQSPATLSVSPGERATLSCRASQSVSSNLAWYQQKPGQAPRLLI | 244 |
| | | YGASTRATGIPARFSGSGSGTEFTLTISSLQSEDFAVYYCQQYDNWWT | |
| | | FGQGTKVEIKRTVAAPSVFIFPPSDEQLKSGTASVVCLLNNFYPREAK | |
| | | VQWKVDN | |
| | Light Chain | EIVMTQSPATLSVSPGERATLSCRASQSVSSNLAWYQQKPGQAPRLLI | 245 |
| | Variable | YGASTRATGIPARFSGSGSGTEFTLTISSLQSEDFAVYYCQQYDNWWT | |
| | Region | FGQGTKVEIK | |
| | LCDR1 | RASQSVSSNLA | 246 |
| | LCDR2 | GASTRAT | 247 |
| | LCDR3 | QQYDNWWT | 248 |
| | LFRW1 | EIVMTQSPATLSVSPGERATLSC | 249 |
| | LFRW2 | WYQQKPGQAPRLLIY | 250 |
| | LFRW3 | GIPARFSGSGSGTEFTLTISSLQSEDFAVYYC | 251 |
| | LFRW4 | FGQGTKVEIK | 252 |
| S24-472 | Heavy Chain | QVQLQESGPGLVKPSGTLSLTCAVSGGSISSINWWSWVRQPPGKGLE | 253 |
| (ORF8) | | WIGEIYHSGNTNYNPSLKSRVTISGDKSKNQFSLKLSSVTAADTAVYY | |
| | | CARGYYDSSPYYEPQGIDYWGQGILVTVSSASTKGPSVFPLAPSSKST | |
| | | SGGTAALGCLVKDYFPEPVTVSWNSGALTSGVHTFPAVLQSSG | |
| | Heavy Chain | QVQLQESGPGLVKPSGTLSLTCAVSGGSISSINWWSWVRQPPGKGLE | 254 |
| | Variable | WIGEIYHSGNTNYNPSLKSRVTISGDKSKNQFSLKLSSVTAADTAVYY | |
| | Region | CARGYYDSSPYYEPQGIDYWGQGILVTVSS | |
| | HCDR1 | SINWWS | 255 |
| | HCDR2 | EIYHSGNTNYNPSLKS | 256 |
| | HCDR3 | GYYDSSPYYEPQGIDY | 257 |
| | HFRW1 | QVQLQESGPGLVKPSGTLSLTCAVSGGSIS | 258 |
| | HFRW2 | WVRQPPGKGLEWIG | 259 |
| | HFRW3 | RVTISGDKSKNQFSLKLSSVTAADTAVYYCAR | 260 |
| | HFRW4 | WGQGILVTVSS | 261 |
| | Light Chain | QLVLTQSPSASASLGASVKLTCTLSSGHSSYTIAWHQQQPEKGPRYL | 262 |
| | | MKVNSDGSHTKGDGIPDRFSGSSSGAERYLTISSLQSEDEADYYCQT | |
| | | WGTGIRVFGGGTKLTVLGQPKAAPSVTLFPPSSEELQANKATLVCLIS | |
| | | DFYPGAVTVAWKADSSPVKAGVETTTPSKQSNNKYAASS | |
| | Light Chain | QLVLTQSPSASASLGASVKLTCTLSSGHSSYTIAWHQQQPEKGPRYL | 263 |
| | Variable | MKVNSDGSHTKGDGIPDRFSGSSSGAERYLTISSLQSEDEADYYCQT | |
| | Region | WGTGIRVFGGGTKLTVL | |
| | LCDR1 | TLSSGHSSYTIA | 264 |
| | LCDR2 | VNSDGSHTKGD | 265 |
| | LCDR3 | QTWGTGIRV | 266 |
| | LFRW1 | QLVLTQSPSASASLGASVKLTC | 267 |
| | LFRW2 | WHQQQPEKGPRYLMK | 268 |
| | LFRW3 | GIPDRFSGSSSGAERYLTISSLQSEDEADYYC | 269 |
| | LFRW4 | FGGGTKLTVL | 270 |
| S24-490 | Heavy Chain | QVQLVQSGAEVKKPGASVKVSCKASGYTFTSYFIHWVRQAPGQGLE | 271 |
| (ORF8) | | WMGIINPSGGSTSYAQKFQGRVTMTRDTSTSTVYMELSSLRSEDTAV | |
| | | YYCARHTTPTRYFDYWGQGTLVTVSSGSASAPTLFPLVSCENSPSDTS | |
| | | SV | |
| | Heavy Chain | QVQLVQSGAEVKKPGASVKVSCKASGYTFTSYFIHWVRQAPGQGLE | 272 |
| | Variable | WMGIINPSGGSTSYAQKFQGRVTMTRDTSTSTVYMELSSLRSEDTAV | |
| | Region | YYCARHTTPTRYFDYWGQGTLVTVSS | |
| | HCDR1 | SYFIH | 273 |
| | HCDR2 | IINPSGGSTSYAQKFQG | 274 |
| | HCDR3 | HTTPTRYFDY | 275 |
| | HFRW1 | QVQLVQSGAEVKKPGASVKVSCKASGYTFT | 276 |
| | HFRW2 | WVRQAPGQGLEWMG | 277 |
| | HFRW3 | RVTMTRDTSTSTVYMELSSLRSEDTAVYYCAR | 278 |
| | HFRW4 | WGQGTLVTVSS | 279 |
| | Light Chain | EIVLTQSPGTLSLSPGERATLSCRASQSVTSSYLAWYQQRRGQAPRLLI | 280 |
| | | YGASSRATGIPDRFSGSGSGTDFTLTISRLEPEDFAVYYCQQYGSSPLT | |
| | | FGGGTKVEIKRTVAAPSVFIFPPSDEQLKSGTASVVCLLNNFYPREAK | |
| | | VQWKVDN | |
| | Light Chain | EIVLTQSPGTLSLSPGERATLSCRASQSVTSSYLAWYQQRRGQAPRLLI | 281 |
| | Variable | YGASSRATGIPDRFSGSGSGTDFTLTISRLEPEDFAVYYCQQYGSSPLT | |
| | Region | FGGGTKVEIK | |
| | LCDR1 | RASQSVTSSYLA | 282 |
| | LCDR2 | GASSRAT | 283 |
| | LCDR3 | QQYGSSPLT | 284 |
| | LFRW1 | EIVLTQSPGTLSLSPGERATLSC | 285 |
| | LFRW2 | WYQQRRGQAPRLLIY | 286 |
| | LFRW3 | GIPDRFSGSGSGTDFTLTISRLEPEDFAVYYC | 287 |
| | LFRW4 | FGGGTKVEIK | 288 |
| S24-494 | Heavy Chain | QLQLQESGPGLVKPSETLSLTCTVSGGSISSSSYYWGWIRQPPGKGLE | 289 |
| (Spike/ | | WIGSIYYSGSTYYNPSLKSRVTISVDTSKNQFSLKLSSVTAADTAVYY | |
| RBD) | | CARKPRSDYGYFDLWGRGTLVTVSSASTKGPSV | |
| | Heavy Chain | QLQLQESGPGLVKPSETLSLTCTVSGGSISSSSYYWGWIRQPPGKGLE | 290 |
| | Variable | WIGSIYYSGSTYYNPSLKSRVTISVDTSKNQFSLKLSSVTAADTAVYY | |
| | Region | CARKPRSDYGYFDLWGRGTLVTVSS | |
| | HCDR1 | SSSYYWG | 291 |
| | HCDR2 | SIYYSGSTYYNPSLKS | 292 |
| | HCDR3 | KPRSDYGYFDL | 293 |
| | HFRW1 | QLQLQESGPGLVKPSETLSLTCTVSGGSIS | 294 |
| | HFRW2 | WIRQPPGKGLEWIG | 295 |
| | HFRW3 | RVTISVDTSKNQFSLKLSSVTAADTAVYYCAR | 296 |
| | HFRW4 | WGRGTLVTVSS | 297 |
| | Light Chain | DIQMTQSPSSLSASVGDRVTITCRASQSISSYLNWYQQKPGKAPKLLIY | 298 |
| | | AASSLQSGVPSRFSGSGSGTDFTLTISSLQPEDFATYYCQQSYSTPQLT | |
| | | FGGGTKVEIKRTVAAPSVFIFPPSDEQLKSGTASVVCLLNNFYPREAK | |
| | | VQWKVDN | |
| | Light Chain | DIQMTQSPSSLSASVGDRVTITCRASQSISSYLNWYQQKPGKAPKLLIY | 299 |
| | Variable | AASSLQSGVPSRFSGSGSGTDFTLTISSLQPEDFATYYCQQSYSTPQLT | |
| | Region | FGGGTKVEIK | |
| | LCDR1 | RASQSISSYLN | 300 |
| | LCDR2 | AASSLQS | 301 |
| | LCDR3 | QQSYSTPQLT | 302 |
| | LFRW1 | DIQMTQSPSSLSASVGDRVTITC | 303 |
| | LFRW2 | WYQQKPGKAPKLLIY | 304 |
| | LFRW3 | GVPSRFSGSGSGTDFTLTISSLQPEDFATYYC | 305 |
| | LFRW4 | FGGGTKVEIK | 306 |
| S24-566 | Heavy Chain | EVQLVESGGGLVKPGRSLRLSCTASGFTFGDYAMSWFRQAPGKGLE | 307 |
| (ORF8) | | WVGFTRRKAYGGTTEYAASVKGRFTISRDDSKSIAYLQMNSLKTEDT | |
| | | AVYYCTRIKVGRFDLTDSGSYRYFDYWGQGTLVTVSSASTKGPSVFP | |
| | | LAPSSKSTSGGTAALGCLVKDYFPEPVTVSWNSGALTSGVHTFPAVL | |
| | | QSSG | |
| | Heavy Chain | EVQLVESGGGLVKPGRSLRLSCTASGFTFGDYAMSWFRQAPGKGLE | 308 |
| | Variable | WVGFTRRKAYGGTTEYAASVKGRFTISRDDSKSIAYLQMNSLKTEDT | |
| | Region | AVYYCTRIKVGRFDLTDSGSYRYFDYWGQGTLVTVSS | |
| | HCDR1 | DYAMS | 309 |
| | HCDR2 | FTRRKAYGGTTEYAASVKG | 310 |
| | HCDR3 | IKVGRFDLTDSGSYRYFDY | 311 |
| | HFRW1 | EVQLVESGGGLVKPGRSLRLSCTASGFTFG | 312 |
| | HFRW2 | WFRQAPGKGLEWVG | 313 |
| | HFRW3 | RFTISRDDSKSIAYLQMNSLKTEDTAVYYCTR | 314 |
| | HFRW4 | WGQGTLVTVSS | 315 |
| | Light Chain | DIVMTQSPLSLPVTPGEPASISCRSSQSLLHSNGYNYLDWYLQKPGQS | 316 |
| | | PQLLIYLGSNRASGVPDRFSGSGSGTDFTLKISRVEAEDVGVYYCMQP | |
| | | LQTPWTFGQGTKVEIKRTVAAPSVFIFPPSDEQLKSGTASVVCLLNNF | |
| | | YPREAKVQWKVDN | |
| | Light Chain | DIVMTQSPLSLPVTPGEPASISCRSSQSLLHSNGYNYLDWYLQKPGQS | 317 |
| | Variable | PQLLIYLGSNRASGVPDRFSGSGSGTDFTLKISRVEAEDVGVYYCMQP | |
| | Region | LQTPWTFGQGTKVEIK | |
| | LCDR1 | RSSQSLLHSNGYNYLD | 318 |
| | LCDR2 | LGSNRAS | 319 |
| | LCDR3 | MQPLQTPWT | 320 |
| | LFRW1 | DIVMTQSPLSLPVTPGEPASISC | 321 |
| | LFRW2 | WYLQKPGQSPQLLIY | 322 |
| | LFRW3 | GVPDRFSGSGSGTDFTLKISRVEAEDVGVYYC | 323 |
| | LFRW4 | FGQGTKVEIK | 324 |
| S24-636 | Heavy Chain | EVQLVESGGGLVQPGGSLRLSCAASGFTLSSYWMSWVRQAPGKGLE | 325 |
| (20) | | WVANIKQDGSEKYYVDSVKGRFTISRDNAKNSLYLQMNSLRAEDTA | |
| | | VYYCARDLTATWFDPWGQGTLVTVSSAPTKAPDVFPIISGCRHPKDN | |
| | | SPVVLACLITGYH | |
| | Heavy Chain | EVQLVESGGGLVQPGGSLRLSCAASGFTLSSYWMSWVRQAPGKGLE | 326 |
| | Variable | WVANIKQDGSEKYYVDSVKGRFTISRDNAKNSLYLQMNSLRAEDTA | |
| | Region | VYYCARDLTATWFDPWGQGTLVTVSS | |
| | HCDR1 | SYWMS | 327 |
| | HCDR2 | NIKQDGSEKYYVDSVKG | 328 |
| | HCDR3 | DLTATWFDP | 329 |
| | HFRW1 | EVQLVESGGGLVQPGGSLRLSCAASGFTLS | 330 |
| | HFRW2 | WVRQAPGKGLEWVA | 331 |
| | HFRW3 | RFTISRDNAKNSLYLQMNSLRAEDTAVYYCAR | 332 |
| | HFRW4 | WGQGTLVTVSS | 333 |
| | Light Chain | QTVVTQEPSFSVSPGGTVTLTCGLSSGSVSTSYYPSWYQQTPGQAPRT | 334 |
| | | LIYSTNKRSSGVPDRFSGSILGNKAALTITGAQADDESDYYCVLYMGS | |
| | | GMSVFGGGTKLTVLGQPKAAPSVTLFPPSSEELQANKATLVCLISDFY | |
| | | PGAVTVAWKADSSPVKAGVETTTPSKQSNNKYAASS | |
| | Light Chain | QTVVTQEPSFSVSPGGTVTLTCGLSSGSVSTSYYPSWYQQTPGQAPRT | 335 |
| | Variable | LIYSTNKRSSGVPDRFSGSILGNKAALTITGAQADDESDYYCVLYMGS | |
| | Region | GMSVFGGGTKLTVL | |
| | LCDR1 | GLSSGSVSTSYYPS | 336 |
| | LCDR2 | STNKRSS | 337 |
| | LCDR3 | VLYMGSGMSV | 338 |
| | LFRW1 | QTVVTQEPSFSVSPGGTVTLTC | 339 |
| | LFRW2 | WYQQTPGQAPRTLIY | 340 |
| | LFRW3 | GVPDRFSGSILGNKAALTITGAQADDESDYYC | 341 |
| | LFRW4 | FGGGTKLTVL | 342 |
| S24-740 | Heavy Chain | QVQLVQSGAEVKKPGASVKVSCKASGYTFTSYALHWVRQAPGQRLE | 343 |
| (ORF8) | | WMGWINAGNGNTKYSQRFQGRVTIIRDTSASTTYMELSSLRSEDTAV | |
| | | YYCARGYARAGVITIKESLHHWGQGTLVTVSSASTKGPSVFPLAPSSK | |
| | | STSGGTAALGCLVKDYFPEPVTVSWNSGALTSGVHTFPAVLQSSG | |
| | Heavy Chain | QVQLVQSGAEVKKPGASVKVSCKASGYTFTSYALHWVRQAPGQRLE | 344 |
| | Variable | WMGWINAGNGNTKYSQRFQGRVTIIRDTSASTTYMELSSLRSEDTAV | |
| | Region | YYCARGYARAGVITIKESLHHWGQGTLVTVSS | |
| | HCDR1 | SYALH | 345 |
| | HCDR2 | WINAGNGNTKYSQRFQG | 346 |
| | HCDR3 | GYARAGVITIKESLHH | 347 |
| | HFRW1 | QVQLVQSGAEVKKPGASVKVSCKASGYTFT | 348 |
| | HFRW2 | WVRQAPGQRLEWMG | 349 |
| | HFRW3 | RVTIIRDTSASTTYMELSSLRSEDTAVYYCAR | 350 |
| | HFRW4 | WGQGTLVTVSS | 351 |
| | Light Chain | DIVMTQSPDSLAVSLGERATINCKSSQSVLYSSNNKNYLAWYQQKPG | 352 |
| | | QPPKLLIYWASTRESGVPDRFSGSGSGTDFTLTISSLQAEDVAVYYCQ | |
| | | QYYSTPPLTFGGGTKVEIKRTVAAPSVFIFPPSDEQLKSGTASVVCLLN | |
| | | NFYPREAKVQWKVDN | |
| | Light Chain | DIVMTQSPDSLAVSLGERATINCKSSQSVLYSSNNKNYLAWYQQKPG | 353 |
| | Variable | QPPKLLIYWASTRESGVPDRFSGSGSGTDFTLTISSLQAEDVAVYYCQ | |
| | Region | QYYSTPPLTFGGGTKVEIK | |
| | LCDR1 | KSSQSVLYSSNNKNYLA | 354 |
| | LCDR2 | WASTRES | 355 |
| | LCDR3 | QQYYSTPPLT | 356 |
| | LFRW1 | DIVMTQSPDSLAVSLGERATINC | 357 |
| | LFRW2 | WYQQKPGQPPKLLIY | 358 |
| | LFRW3 | GVPDRFSGSGSGTDFTLTISSLQAEDVAVYYC | 359 |
| | LFRW4 | FGGGTKVEIK | 360 |
| S24-791 | Heavy Chain | QVQLQESGPGLVKPSETLSLTCTVSGGSISSSYWSWIRQPPGKGLEWI | 361 |
| (NP) | | GYIYYSGNTNYNPSLKSRVTLSIDTSKNQFSLKLSSVTAADTAVYYCA | |
| | | CSVTIFGVVTPAFDIWGQGTMVTVSSASTKGPSVFPLAPSSKSTSGGT | |
| | | AALGCLVKDYFPEPVTVSWNSGALTSGVHTFPAVLQSSG | |
| | Heavy Chain | QVQLQESGPGLVKPSETLSLTCTVSGGSISSSYWSWIRQPPGKGLEWI | 362 |
| | Variable | GYIYYSGNTNYNPSLKSRVTLSIDTSKNQFSLKLSSVTAADTAVYYCA | |
| | Region | CSVTIFGVVTPAFDIWGQGTMVTVSS | |
| | HCDR1 | SSYWS | 363 |
| | HCDR2 | YIYYSGNTNYNPSLKS | 364 |
| | HCDR3 | SVTIFGVVTPAFDI | 365 |
| | HFRW1 | QVQLQESGPGLVKPSETLSLTCTVSGGSIS | 366 |
| | HFRW2 | WIRQPPGKGLEWIG | 367 |
| | HFRW3 | RVTLSIDTSKNQFSLKLSSVTAADTAVYYCAC | 368 |
| | HFRW4 | WGQGTMVTVSS | 369 |
| | Light Chain | EIVLTHSPGTLSLSPGERATLSCRASQSVRSYLAWYQQKPGQAPRLLI | 370 |
| | | YGASSRATGIPDRFSGSGSGTDFTLTISRLEPDDFAVYYCQQYGSSPW | |
| | | TFGQGTKVEIKRTVAAPSVFIFPPSDEQLKSGTASVVCLLNNFYPREA | |
| | | KVQWKVDNALQSGNSQESVTEQDSKDSTYSLSSTLTLSKADYE | |
| | Light Chain | EIVLTHSPGTLSLSPGERATLSCRASQSVRSYLAWYQQKPGQAPRLLI | 371 |
| | Variable | YGASSRATGIPDRFSGSGSGTDFTLTISRLEPDDFAVYYCQQYGSSPW | |
| | Region | TFGQGTKVEIK | |
| | LCDR1 | RASQSVRSYLA | 372 |
| | LCDR2 | GASSRAT | 373 |
| | LCDR3 | QQYGSSPWT | 374 |
| | LFRW1 | EIVLTHSPGTLSLSPGERATLSC | 375 |
| | LFRW2 | WYQQKPGQAPRLLIY | 376 |
| | LFRW3 | GIPDRFSGSGSGTDFTLTISRLEPDDFAVYYC | 377 |
| | LFRW4 | FGQGTKVEIK | 378 |
| S24-902 | Heavy Chain | QVQLVQSGAEVKKPGSSVKVSCKASGGTFSSYAISWVRQAPGQGLE | 379 |
| (Spike/ | | WMGRIIPILGIANYAQKFQGRVTITADKSTSTAYMELSSLRSEDTAVY | |
| RBD) | | YCARWDFGVVIQYGMDVWGQGTTVTVSSASTKGPSVFPLAPSSKSTS | |
| | | GGTAALGCLVKDYFPEPVTVSWNSGALTSGVHTFPAVLQSSGLYSLS | |
| | | SVVTVPSSSL | |
| | Heavy Chain | QVQLVQSGAEVKKPGSSVKVSCKASGGTFSSYAISWVRQAPGQGLE | 380 |
| | Variable | WMGRIIPILGIANYAQKFQGRVTITADKSTSTAYMELSSLRSEDTAVY | |
| | Region | YCARWDFGVVIQYGMDVWGQGTTVTVSS | |
| | HCDR1 | SYAIS | 381 |
| | HCDR2 | RIIPILGIANYAQKFQG | 382 |
| | HCDR3 | WDFGVVIQYGMDV | 383 |
| | HFRW1 | QVQLVQSGAEVKKPGSSVKVSCKASGGTFS | 384 |
| | HFRW2 | WVRQAPGQGLEWMG | 385 |
| | HFRW3 | RVTITADKSTSTAYMELSSLRSEDTAVYYCAR | 386 |
| | HFRW4 | WGQGTTVTVSS | 387 |
| | Light Chain | QAVVTQEPSLTVSPGGTVTLTCGSSTGAVTSGHYPYWFQQKPGQAPR | 388 |
| | | TLIYDTSNKHSWTPARFSGSLLGGKAALTLSGAQPEDEAEYYCLLSYS | |
| | | GWVFGGGTKLTVLGQPKAAPSVTLFPPSSEELQANKATLVCLISDFYP | |
| | | GAVTVAWKADSSPVKAGVETTTPSKQSNNKYAASS | |
| | Light Chain | QAVVTQEPSLTVSPGGTVTLTCGSSTGAVTSGHYPYWFQQKPGQAPR | 389 |
| | Variable | TLIYDTSNKHSWTPARFSGSLLGGKAALTLSGAQPEDEAEYYCLLSYS | |
| | Region | GWVFGGGTKLTVL | |
| | LCDR1 | GSSTGAVTSGHYPY | 390 |
| | LCDR2 | DTSNKHS | 391 |
| | LCDR3 | LLSYSGWV | 392 |
| | LFRW1 | QAVVTQEPSLTVSPGGTVTLTC | 393 |
| | LFRW2 | WFQQKPGQAPRTLIY | 394 |
| | LFRW3 | WTPARFSGSLLGGKAALTLSGAQPEDEAEYYC | 395 |
| | LFRW4 | FGGGTKLTVL | 396 |
| S24-921 | Heavy Chain | QVQLQESGPGLVKPSETLSLTCTVSGGSINSFYWNWIRQPPGKGLEWI | 397 |
| (NP) | | GYIYYSGNTKYNPSLKSRVTISVDTSNSQFSLKLSSVTAADTAVYYCA | |
| | | ALKKQELVSLQAFDIWGQGTMVTVSSASTKGPSVFPLAPSSKSTSGGT | |
| | | AALGCLVKDYFPEPVTVSWNSGALTSGVHTFPAVLQSSG | |
| | Heavy Chain | QVQLQESGPGLVKPSETLSLTCTVSGGSINSFYWNWIRQPPGKGLEWI | 398 |
| | Variable | GYIYYSGNTKYNPSLKSRVTISVDTSNSQFSLKLSSVTAADTAVYYCA | |
| | Region | ALKKQELVSLQAFDIWGQGTMVTVSS | |
| | HCDR1 | SFYWN | 399 |
| | HCDR2 | YIYYSGNTKYNPSLKS | 400 |
| | HCDR3 | LKKQELVSLQAFDI | 401 |
| | HFRW1 | QVQLQESGPGLVKPSETLSLTCTVSGGSIN | 402 |
| | HFRW2 | WIRQPPGKGLEWIG | 403 |
| | HFRW3 | RVTISVDTSNSQFSLKLSSVTAADTAVYYCAA | 404 |
| | HFRW4 | WGQGTMVTVSS | 405 |
| | Light Chain | DIQMTQSPSSLSASLGDGVTITCRASQSISSYLSWYQQKPGKAPKLLIY | 406 |
| | | AASSLQSGVPSRFSGSGSGTDFTLTISSLQPEDFATYYCQQSYNTPVTF | |
| | | GQGTKVEIKRTVAAPSVFIFPPSDEQLKSGTASVVCLLNNFYPREAKV | |
| | | QWKVDNADRKS | |
| | Light Chain | DIQMTQSPSSLSASLGDGVTITCRASQSISSYLSWYQQKPGKAPKLLIY | 407 |
| | Variable | AASSLQSGVPSRFSGSGSGTDFTLTISSLQPEDFATYYCQQSYNTPVTF | |
| | Region | GQGTKVEIK | |
| | LCDR1 | RASQSISSYLS | 408 |
| | LCDR2 | AASSLQS | 409 |
| | LCDR3 | QQSYNTPVT | 410 |
| | LFRW1 | DIQMTQSPSSLSASLGDGVTITC | 411 |
| | LFRW2 | WYQQKPGKAPKLLIY | 412 |
| | LFRW3 | GVPSRFSGSGSGTDFTLTISSLQPEDFATYYC | 413 |
| | LFRW4 | FGQGTKVEIK | 414 |
| S24-1063 | Heavy Chain | QVQLQESGPGLVKPSETLSLTCTVSGGSISSYYWSWIRQPPGKGLEWI | 415 |
| (NP) | | GYIYYSGSTKYNPSLKSRVTISVDTSKNQFSLKLTSVTAADTAVYYCA | |
| | | RIYDSSGYYHPVFDYWGQGTLVTVSSASTKGPSVFPLAPSSKSTSGGT | |
| | | AALGCLVKDYFPEPVTVSWNSGALTSGVHTFPAVLQSSG | |
| | Heavy Chain | QVQLQESGPGLVKPSETLSLTCTVSGGSISSYYWSWIRQPPGKGLEWI | 416 |
| | Variable | GYIYYSGSTKYNPSLKSRVTISVDTSKNQFSLKLTSVTAADTAVYYCA | |
| | Region | RIYDSSGYYHPVFDYWGQGTLVTVSS | |
| | HCDR1 | SYYWS | 417 |
| | HCDR2 | YIYYSGSTKYNPSLKS | 418 |
| | HCDR3 | IYDSSGYYHPVFDY | 419 |
| | HFRW1 | QVQLQESGPGLVKPSETLSLTCTVSGGSIS | 420 |
| | HFRW2 | WIRQPPGKGLEWIG | 421 |
| | HFRW3 | RVTISVDTSKNQFSLKLTSVTAADTAVYYCAR | 422 |
| | HFRW4 | WGQGTLVTVSS | 423 |
| | Light Chain | EIVLTQSPGTLSLSPGERATLSCRASQSVSSSYLAWYQQKPGQAPRLLI | 424 |
| | | YGASSRATDIPDRFSGSGSGTDFTLTISRLEPEDFAVYYCQQYGSSPWT | |
| | | FGQGTKVEIKRTVAAPSVFIFPPSDEQLKSGTASVVCLLNNFYPREAK | |
| | | VQWKVDN | |
| | Light Chain | EIVLTQSPGTLSLSPGERATLSCRASQSVSSSYLAWYQQKPGQAPRLLI | 425 |
| | Variable | YGASSRATDIPDRFSGSGSGTDFTLTISRLEPEDFAVYYCQQYGSSPWT | |
| | Region | FGQGTKVEIK | |
| | LCDR1 | RASQSVSSSYLA | 426 |
| | LCDR2 | GASSRAT | 427 |
| | LCDR3 | QQYGSSPWT | 428 |
| | LFRW1 | EIVLTQSPGTLSLSPGERATLSC | 429 |
| | LFRW2 | WYQQKPGQAPRLLIY | 430 |
| | LFRW3 | DIPDRFSGSGSGTDFTLTISRLEPEDFAVYYC | 431 |
| | LFRW4 | FGQGTKVEIK | 432 |
| S24-1224 | Heavy Chain | QVQLVQSGAEVKKPGASVRVSCKASGYTFTSYYIYWVRQAPGQGLE | 433 |
| (Spike/ | | WMGVINPSGGSTSYAQKFQGRVTLTRDTSTSTVYMDLSSLRSEDTAV | |
| RBD) | | YYCARDPIMWEVVTRGRGNWFDPWGQGTLVTVSSASTKGPSVFPLA | |
| | | PSSKSTSGGTAALGCLVKDYFPEPVTVSWNSGALTSGVHTFPAVLQSS | |
| | | G | |
| | Heavy Chain | QVQLVQSGAEVKKPGASVRVSCKASGYTFTSYYIYWVRQAPGQGLE | 434 |
| | Variable | WMGVINPSGGSTSYAQKFQGRVTLTRDTSTSTVYMDLSSLRSEDTAV | |
| | Region | YYCARDPIMWEVVTRGRGNWFDPWGQGTLVTVSS | |
| | HCDR1 | SYYIY | 435 |
| | HCDR2 | VINPSGGSTSYAQKFQG | 436 |
| | HCDR3 | DPIMWEVVTRGRGNWFDP | 437 |
| | HFRW1 | QVQLVQSGAEVKKPGASVRVSCKASGYTFT | 438 |
| | HFRW2 | WVRQAPGQGLEWMG | 439 |
| | HFRW3 | RVTLTRDTSTSTVYMDLSSLRSEDTAVYYCAR | 440 |
| | HFRW4 | WGQGTLVTVSS | 441 |
| | Light Chain | QSVLTQPPSVSGAPGQRVTIPCTGSSFNIGAGYDVHWYQQLPGTAPKL | 442 |
| | | LIFGNSNRPSGVPDRFSGSRSGTSASLAITGLQAEDEADYYCQSYDSSL | |
| | | SGVVFGGGTTLTVLGQPKAAPSVTLFPPSSEELQANKATLVCLISDFY | |
| | | PGAVTVAWKADSSPVKAGVETTTPSKQSNNKYAASSYLSLTPEQWK | |
| | | SH | |
| | Light Chain | QSVLTQPPSVSGAPGQRVTIPCTGSSFNIGAGYDVHWYQQLPGTAPKL | 443 |
| | Variable | LIFGNSNRPSGVPDRFSGSRSGTSASLAITGLQAEDEADYYCQSYDSSL | |
| | Region | SGVVFGGGTTLTVL | |
| | LCDR1 | TGSSFNIGAGYDVH | 444 |
| | LCDR2 | GNSNRPS | 445 |
| | LCDR3 | QSYDSSLSGVV | 446 |
| | LFRW1 | QSVLTQPPSVSGAPGQRVTIPC | 447 |
| | LFRW2 | WYQQLPGTAPKLLIF | 448 |
| | LFRW3 | GVPDRFSGSRSGTSASLAITGLQAEDEADYYC | 449 |
| | LFRW4 | FGGGTTLTVL | 450 |
| S24-1271 | Heavy Chain | EVQLVESGGGLVQPGGSLRLSCAASGFTVSSNYMSWVRQAPGKGLE | 451 |
| (Spike/ | | WVSVIYSDGNTYYADSVKGRFTISRDNSKNMLYLQMNSLRAEDTAV | |
| RBD) | | YYCARDPGQGYCSGGSCAPSYSLDYWGQGTLVTVSSGSASAPTLFPL | |
| | | VSCENSPSDTSSV | |
| | Heavy Chain | EVQLVESGGGLVQPGGSLRLSCAASGFTVSSNYMSWVRQAPGKGLE | 452 |
| | Variable | WVSVIYSDGNTYYADSVKGRFTISRDNSKNMLYLQMNSLRAEDTAV | |
| | Region | YYCARDPGQGYCSGGSCAPSYSLDYWGQGTLVTVSS | |
| | HCDR1 | SNYMS | 453 |
| | HCDR2 | VIYSDGNTYYADSVKG | 454 |
| | HCDR3 | DPGQGYCSGGSCAPSYSLDY | 455 |
| | HFRW1 | EVQLVESGGGLVQPGGSLRLSCAASGFTVS | 456 |
| | HFRW2 | WVRQAPGKGLEWVS | 457 |
| | HFRW3 | RFTISRDNSKNMLYLQMNSLRAEDTAVYYCAR | 458 |
| | HFRW4 | WGQGTLVTVSS | 459 |
| | Light Chain | SYELTQPPSVSVSPGQTASITCSGDKLGDRYVCWYQQKPGQSPVLVIY | 460 |
| | | QDTKRPSGIPERFSGSNSGNTATLTISGTQAMDEADYYCQAWDSSTW | |
| | | VFGGGTKLTVLGQPKAAPSVTLFPPSSEELQANKATLVCLISDFYPGA | |
| | | VTVAWKADSSPVKAGVETTTPSKQSNNKYAASS | |
| | Light Chain | SYELTQPPSVSVSPGQTASITCSGDKLGDRYVCWYQQKPGQSPVLVIY | 461 |
| | Variable | QDTKRPSGIPERFSGSNSGNTATLTISGTQAMDEADYYCQAWDSSTW | |
| | Region | VFGGGTKLTVL | |
| | LCDR1 | SGDKLGDRYVC | 462 |
| | LCDR2 | QDTKRPS | 463 |
| | LCDR3 | QAWDSSTWV | 464 |
| | LFRW1 | SYELTQPPSVSVSPGQTASITC | 465 |
| | LFRW2 | WYQQKPGQSPVLVIY | 466 |
| | LFRW3 | GIPERFSGSNSGNTATLTISGTQAMDEADYYC | 467 |
| | LFRW4 | FGGGTKLTVL | 468 |
| S24-1339 | Heavy Chain | EVQLVESGGGLVQPGGSLRLSCAASGFTVSSNYMSWVRQAPGKGLE | 469 |
| (Spike/ | | WVSDIYSGGSTYYADSVKGRFTISRHNSKNTLYLQMNSLRAEDTAVY | |
| RBD) | | YCARDRRGYSYGLHHGMDVWGQGTTVTVSSASTKGPSVFPLAPSSK | |
| | | STSGGTAALGCLVKDYFPEPVTVSWNSGALTSGVHTFPAVLQSSG | |
| | Heavy Chain | EVQLVESGGGLVQPGGSLRLSCAASGFTVSSNYMSWVRQAPGKGLE | 470 |
| | Variable | WVSDIYSGGSTYYADSVKGRFTISRHNSKNTLYLQMNSLRAEDTAVY | |
| | Region | YCARDRRGYSYGLHHGMDVWGQGTTVTVSS | |
| | HCDR1 | SNYMS | 471 |
| | HCDR2 | DIYSGGSTYYADSVKG | 472 |
| | HCDR3 | DRRGYSYGLHHGMDV | 473 |
| | HFRW1 | EVQLVESGGGLVQPGGSLRLSCAASGFTVS | 474 |
| | HFRW2 | WVRQAPGKGLEWVS | 475 |
| | HFRW3 | RFTISRHNSKNTLYLQMNSLRAEDTAVYYCAR | 476 |
| | HFRW4 | WGQGTTVTVSS | 477 |
| | Light Chain | EIVLTQSPGTLSLSPGERATLSCRASQSVSSSYLAWYQQKPDQAPRLLI | 478 |
| | | YGASSRATGIPDRFSGSGSGTDFTLTISRLEPEDFAVYYCQQYGSSPNT | |
| | | FGQGTKLEIKRTVAAPSVFIFPPSDEQLKSGTASVVCLLNNFYPREAK | |
| | | VQWKVDN | |
| | Light Chain | EIVLTQSPGTLSLSPGERATLSCRASQSVSSSYLAWYQQKPDQAPRLLI | 479 |
| | Variable | YGASSRATGIPDRFSGSGSGTDFTLTISRLEPEDFAVYYCQQYGSSPNT | |
| | Region | FGQGTKLEIK | |
| | LCDR1 | RASQSVSSSYLA | 480 |
| | LCDR2 | GASSRAT | 481 |
| | LCDR3 | QQYGSSPNT | 482 |
| | LFRW1 | EIVLTQSPGTLSLSPGERATLSC | 483 |
| | LFRW2 | WYQQKPDQAPRLLIY | 484 |
| | LFRW3 | GIPDRFSGSGSGTDFTLTISRLEPEDFAVYYC | 485 |
| | LFRW4 | FGQGTKLEIK | 486 |
| S24-1345 | Heavy Chain | QLQLQESGPGLVKPSETLSLTCTVSGGSISSSSYYWGWIRQPPGKGLE | 487 |
| (Spike/ | | WIGSIYYSGSTYYNPSLKSRVTISVDTSKNQFSLKLSSVTAADTAVYY | |
| RBD) | | CARRIRRPTSEVVITYVFDYWGQGTLVTVSSAPTKAPDVFPIISGCRHP | |
| | | KDNSPVVLACLITGYH | |
| | Heavy Chain | QLQLQESGPGLVKPSETLSLTCTVSGGSISSSSYYWGWIRQPPGKGLE | 488 |
| | Variable | WIGSIYYSGSTYYNPSLKSRVTISVDTSKNQFSLKLSSVTAADTAVYY | |
| | Region | CARRIRRPTSEVVITYVFDYWGQGTLVTVSS | |
| | HCDR1 | SSSYYWG | 489 |
| | HCDR2 | SIYYSGSTYYNPSLKS | 490 |
| | HCDR3 | RIRRPTSEVVITYVFDY | 491 |
| | HFRW1 | QLQLQESGPGLVKPSETLSLTCTVSGGSIS | 492 |
| | HFRW2 | WIRQPPGKGLEWIG | 493 |
| | HFRW3 | RVTISVDTSKNQFSLKLSSVTAADTAVYYCAR | 494 |
| | HFRW4 | WGQGTLVTVSS | 495 |
| | Light Chain | AIQLTQSPSSLSASVGDRVTITCRASQGISSALAWYQQKPGKAPKLLIY | 496 |
| | | DASSLESGVPSRFSGSGSGTDFTLTISSLQPEDFATYYCQQFNSYLTFG | |
| | | GGTKVEIKRTVAAPSVFIFPPSDEQLKSGTASVVCLLNNFYPREAKVQ | |
| | | WKVDNALQSGNSQESVTEQDSKDSTYSLS | |
| | Light Chain | AIQLTQSPSSLSASVGDRVTITCRASQGISSALAWYQQKPGKAPKLLIY | 497 |
| | Variable | DASSLESGVPSRFSGSGSGTDFTLTISSLQPEDFATYYCQQFNSYLTFG | |
| | Region | GGTKVEIK | |
| | LCDR1 | RASQGISSALA | 498 |
| | LCDR2 | DASSLES | 499 |
| | LCDR3 | QQFNSYLT | 500 |
| | LFRW1 | AIQLTQSPSSLSASVGDRVTITC | 501 |
| | LFRW2 | WYQQKPGKAPKLLIY | 502 |
| | LFRW3 | GVPSRFSGSGSGTDFTLTISSLQPEDFATYYC | 503 |
| | LFRW4 | FGGGTKVEIK | 504 |
| S24-1378 | Heavy Chain | EVQLVESGGGLVQPGGSLRLSCAASGFTVSSNYMSWVRQAPGKGLE | 505 |
| (ORF8) | | WVSVIYSGGSTYYADSVKGRFTISRHNSKNTLYLQMNSLRAEDTAVY | |
| | | YCAREGYCTNGVCYRHAFDIWGQGTMVTVSSGSASAPTLFPLVSCEN | |
| | | SPSDTSSV | |
| | Heavy Chain | EVQLVESGGGLVQPGGSLRLSCAASGFTVSSNYMSWVRQAPGKGLE | 506 |
| | Variable | WVSVIYSGGSTYYADSVKGRFTISRHNSKNTLYLQMNSLRAEDTAVY | |
| | Region | YCAREGYCTNGVCYRHAFDIWGQGTMVTVSS | |
| | HCDR1 | SNYMS | 507 |
| | HCDR2 | VIYSGGSTYYADSVKG | 508 |
| | HCDR3 | EGYCTNGVCYRHAFDI | 509 |
| | HFRW1 | EVQLVESGGGLVQPGGSLRLSCAASGFTVS | 510 |
| | HFRW2 | WVRQAPGKGLEWVS | 511 |
| | HFRW3 | RFTISRHNSKNTLYLQMNSLRAEDTAVYYCAR | 512 |
| | HFRW4 | WGQGTMVTVSS | 513 |
| | Light Chain | QTVVTQEPSFSVSPGGTVTLTCGLSSGSVSTSYYPSWYQQTPGQAPRT | 514 |
| | | LIYSTNTRSSGVPDRFSGSILGNKAALTITGAQADDESDYYCVLYMGS | |
| | | GISVFGGGTKLTVLGQPKAAPSVTLFPPSSEELQANKATLVCLISDFYP | |
| | | GAVTVAWKADSSPVKAGVETTTPSKQSNNKYAASS | |
| | Light Chain | QTVVTQEPSFSVSPGGTVTLTCGLSSGSVSTSYYPSWYQQTPGQAPRT | 515 |
| | Variable | LIYSTNTRSSGVPDRFSGSILGNKAALTITGAQADDESDYYCVLYMGS | |
| | Region | GISVFGGGTKLTVL | |
| | LCDR1 | GLSSGSVSTSYYPS | 516 |
| | LCDR2 | STNTRSS | 517 |
| | LCDR3 | VLYMGSGISV | 518 |
| | LFRW1 | QTVVTQEPSFSVSPGGTVTLTC | 519 |
| | LFRW2 | WYQQTPGQAPRTLIY | 520 |
| | LFRW3 | GVPDRFSGSILGNKAALTITGAQADDESDYYC | 521 |
| | LFRW4 | FGGGTKLTVL | 522 |
| S24-1379 | Heavy Chain | QVQLQESGPGLVKPSETLSLTCTVSGGSISSYYWSWIRQPPGKGLEWI | 523 |
| (NP) | | GYIYYSGSTNYNPSLKSRVTISVDTSKNQFSLKLSSVTAADTAVYYCA | |
| | | RDYYQLPMDVWGQGTTVTVSSASTKGPSVFPLAPSSKSTSGGTAALG | |
| | | CLVKDYFPEPVTVSWNSGALTSGVHTFPAVLQSSG | |
| | Heavy Chain | QVQLQESGPGLVKPSETLSLTCTVSGGSISSYYWSWIRQPPGKGLEWI | 524 |
| | Variable | GYIYYSGSTNYNPSLKSRVTISVDTSKNQFSLKLSSVTAADTAVYYCA | |
| | Region | RDYYQLPMDVWGQGTTVTVSS | |
| | HCDR1 | SYYWS | 525 |
| | HCDR2 | YIYYSGSTNYNPSLKS | 526 |
| | HCDR3 | DYYQLPMDV | 527 |
| | HFRW1 | QVQLQESGPGLVKPSETLSLTCTVSGGSIS | 528 |
| | HFRW2 | WIRQPPGKGLEWIG | 529 |
| | HFRW3 | RVTISVDTSKNQFSLKLSSVTAADTAVYYCAR | 530 |
| | HFRW4 | WGQGTTVTVSS | 531 |
| | Light Chain | QSVLTQPPSASGTPGQRVTISCSGSSSNIGSNYVYWYQQLPGTAPKLLI | 532 |
| | | YRNNQRPSGVPDRFSGSKSGTSASLAISGLRSEDEADYYCAAWDDSL | |
| | | SGRVFGGGTKLTVLGQPKAAPSVTLFPPSSEELQANKATLVCLISDFY | |
| | | PGAVTVAWKADSSPVKAGVETTTPSKQSNNKYAASS | |
| | Light Chain | QSVLTQPPSASGTPGQRVTISCSGSSSNIGSNYVYWYQQLPGTAPKLLI | 533 |
| | Variable | YRNNQRPSGVPDRFSGSKSGTSASLAISGLRSEDEADYYCAAWDDSL | |
| | Region | SGRVFGGGTKLTVL | |
| | LCDR1 | SGSSSNIGSNYVY | 534 |
| | LCDR2 | RNNQRPS | 535 |
| | LCDR3 | AAWDDSLSGRV | 536 |
| | LFRW1 | QSVLTQPPSASGTPGQRVTISC | 537 |
| | LFRW2 | WYQQLPGTAPKLLIY | 538 |
| | LFRW3 | GVPDRFSGSKSGTSASLAISGLRSEDEADYYC | 539 |
| | LFRW4 | FGGGTKLTVL | 540 |
| | Heavy Chain | EVQLVESGGGLVQPGGSLRLSCAVSGFTFSSYSMNWVRQAPGKGLE | 541 |
| | | WVSYISSSSSIIYYADSVKGRFTISRDNAKNSLYLQMNSLRAEDTAVY | |
| | | YCARDFLDYSRSYSYGMDVWGQGTTVTVSSASTKGPSVFPLAPSSKS | |
| | | TSGGTAALGCLVKDYFPEPVTVSWNSGALTSGVHTFPAVLQSSG | |
| | Heavy Chain | EVQLVESGGGLVQPGGSLRLSCAVSGFTFSSYSMNWVRQAPGKGLE | 542 |
| | Variable | WVSYISSSSSIIYYADSVKGRFTISRDNAKNSLYLQMNSLRAEDTAVY | |
| | Region | YCARDFLDYSRSYSYGMDVWGQGTTVTVSS | |
| | HCDR1 | SYSMN | 543 |
| | HCDR2 | YISSSSSIIYYADSVKG | 544 |
| | HCDR3 | DFLDYSRSYSYGMDV | 545 |
| | HFRW1 | EVQLVESGGGLVQPGGSLRLSCAVSGFTFS | 546 |
| | HFRW2 | WVRQAPGKGLEWVS | 547 |
| | HFRW3 | RFTISRDNAKNSLYLQMNSLRAEDTAVYYCAR | 548 |
| | HFRW4 | WGQGTTVTVSS | 549 |
| | Light Chain | SYVLTQPPSVSVAPGQTARITCGGDNIGSKNVHWYQQKPGQAPVLVV | 550 |
| | | FDDSDRPSGIPERFSGSNSGNTATLTISRVEAGDEADYYCQVWDSSSD | |
| | | HYVVFGGGTKLTVLGQPKAAPSVTLFPPSSEELQANKATLVCLISDFY | |
| | | PGAVTVAWKADSSPVKAGVETTTPSKQSNNKYAASSY | |
| | Light Chain | SYVLTQPPSVSVAPGQTARITCGGDNIGSKNVHWYQQKPGQAPVLVV | 551 |
| | Variable | FDDSDRPSGIPERFSGSNSGNTATLTISRVEAGDEADYYCQVWDSSSD | |
| | Region | HYVVFGGGTKLTVL | |
| | LCDR1 | GGDNIGSKNVH | 552 |
| | LCDR2 | DDSDRPS | 553 |
| | LCDR3 | QVWDSSSDHYVV | 554 |
| | LFRW1 | SYVLTQPPSVSVAPGQTARITC | 555 |
| | LFRW2 | WYQQKPGQAPVLVVF | 556 |
| | LFRW3 | GIPERFSGSNSGNTATLTISRVEAGDEADYYC | 557 |
| | LFRW4 | FGGGTKLTVL | 558 |
| S24-1476 | Heavy Chain | EVQLVESGGGLVQPGRSLRLSCTASGFTFGDYAMSWFRQAPGKGLE | 559 |
| (Spike/ | | WVGFIRSKAYGGTTQYAASVKGRFTISRDDSKSIAYLQMNSLKTEDT | |
| RBD) | | AVYYCTRVRYCTNGVCYGYHFDYWGQGTVVTVSSAST | |
| | Heavy Chain | EVQLVESGGGLVQPGRSLRLSCTASGFTFGDYAMSWFRQAPGKGLE | 560 |
| | Variable | WVGFIRSKAYGGTTQYAASVKGRFTISRDDSKSIAYLQMNSLKTEDT | |
| | Region | AVYYCTRVRYCTNGVCYGYHFDYWGQGTVVTVSS | |
| | HCDR1 | DYAMS | 561 |
| | HCDR2 | FIRSKAYGGTTQYAASVKG | 562 |
| | HCDR3 | VRYCTNGVCYGYHFDY | 563 |
| | HFRW1 | EVQLVESGGGLVQPGRSLRLSCTASGFTFG | 564 |
| | HFRW2 | WFRQAPGKGLEWVG | 565 |
| | HFRW3 | RFTISRDDSKSIAYLQMNSLKTEDTAVYYCTR | 566 |
| | HFRW4 | WGQGTVVTVSS | 567 |
| | Light Chain | EIVMTQSPATLSVSPGERATLSCRASQSVSSNLAWYQQKPGQAPRLLI | 568 |
| | | YGASTRATGIPARFSGSGSGTEFTLTISSLQSEDFAVYYCQQYNNWWT | |
| | | FGQGTKVEIKRTVAAPSVFIFPPSDEQLKSGTASVVCLLNNFYPREAK | |
| | | VQWKVDN | |
| | Light Chain | EIVMTQSPATLSVSPGERATLSCRASQSVSSNLAWYQQKPGQAPRLLI | 569 |
| | Variable | YGASTRATGIPARFSGSGSGTEFTLTISSLQSEDFAVYYCQQYNNWWT | |
| | Region | FGQGTKVEIK | |
| | LCDR1 | RASQSVSSNLA | 570 |
| | LCDR2 | GASTRAT | 571 |
| | LCDR3 | QQYNNWWT | 572 |
| | LFRW1 | EIVMTQSPATLSVSPGERATLSC | 573 |
| | LFRW2 | WYQQKPGQAPRLLIY | 574 |
| | LFRW3 | GIPARFSGSGSGTEFTLTISSLQSEDFAVYYC | 575 |
| | LFRW4 | FGQGTKVEIK | 576 |
| S24-1564 | Heavy Chain | QVQLQESGPGLVKPSETLSLTCTVSGGSISSYYWSWIRQPPGKGLEWI | 577 |
| (NP) | | GYVYYSGNTKYNPSLKSRVTISVDTSKNQFSLKLGSVTAADTAVYYC | |
| | | ARHSRIEVAGTLDFDYWGQGTLVTVSSASTKGPSVFPLAPSSKSTSGG | |
| | | TAALGCLVKDYFPEPVTVSWNSGALTSGVHTFPAVLQSSG | |
| | Heavy Chain | QVQLQESGPGLVKPSETLSLTCTVSGGSISSYYWSWIRQPPGKGLEWI | 578 |
| | Variable | GYVYYSGNTKYNPSLKSRVTISVDTSKNQFSLKLGSVTAADTAVYYC | |
| | Region | ARHSRIEVAGTLDFDYWGQGTLVTVSS | |
| | HCDR1 | SYYWS | 579 |
| | HCDR2 | YVYYSGNTKYNPSLKS | 580 |
| | HCDR3 | HSRIEVAGTLDFDY | 581 |
| | HFRW1 | QVQLQESGPGLVKPSETLSLTCTVSGGSIS | 582 |
| | HFRW2 | WIRQPPGKGLEWIG | 583 |
| | HFRW3 | RVTISVDTSKNQFSLKLGSVTAADTAVYYCAR | 584 |
| | HFRW4 | WGQGTLVTVSS | 585 |
| | Light Chain | DIQMTQSPSSLSASVGDRVTITCRASQSIRSYLNWYQQKRGKAPKLLI | 586 |
| | | YAASSLQSGVPSRFSGSGSGTDFTLTISSLQPEDFATYYCQQSYSTPPT | |
| | | FGQGTKVEIKRTVAAPSVFIFPPSDEQLKSGTASVVCLLNNFYPREAK | |
| | | VQWKVDN | |
| | Light Chain | DIQMTQSPSSLSASVGDRVTITCRASQSIRSYLNWYQQKRGKAPKLLI | 587 |
| | Variable | YAASSLQSGVPSRFSGSGSGTDFTLTISSLQPEDFATYYCQQSYSTPPT | |
| | Region | FGQGTKVEIK | |
| | LCDR1 | RASQSIRSYLN | 588 |
| | LCDR2 | AASSLQS | 589 |
| | LCDR3 | QQSYSTPPT | 590 |
| | LFRW1 | DIQMTQSPSSLSASVGDRVTITC | 591 |
| | LFRW2 | WYQQKRGKAPKLLIY | 592 |
| | LFRW3 | GVPSRFSGSGSGTDFTLTISSLQPEDFATYYC | 593 |
| | LFRW4 | FGQGTKVEIK | 594 |
| S24-1636 | Heavy Chain | QVQLVESGGGVVQPGRSLRLSCAASGFTFSNYGMHWVRQAPGKGLE | 595 |
| (NP) | | WVAVIWYDGSNKYYADSVKGRFTISRDNSKNTLYLQMNSLRAEDTA | |
| | | VYYCARGDCTNGVCHPLLIYYDSSGLDYWGQGTLVTVSSASTKGPS | |
| | | VFPLAPSSKSTSGGTAALGCLVKDYFPEPVTVSWNSGALTSGVHTFPA | |
| | | VLQSSG | |
| | Heavy Chain | QVQLVESGGGVVQPGRSLRLSCAASGFTFSNYGMHWVRQAPGKGLE | 596 |
| | Variable | WVAVIWYDGSNKYYADSVKGRFTISRDNSKNTLYLQMNSLRAEDTA | |
| | Region | VYYCARGDCTNGVCHPLLIYYDSSGLDYWGQGTLVTVSS | |
| | HCDR1 | NYGMH | 597 |
| | HCDR2 | VIWYDGSNKYYADSVKG | 598 |
| | HCDR3 | GDCTNGVCHPLLIYYDSSGLDY | 599 |
| | HFRW1 | QVQLVESGGGVVQPGRSLRLSCAASGFTFS | 600 |
| | HFRW2 | WVRQAPGKGLEWVA | 601 |
| | HFRW3 | RFTISRDNSKNTLYLQMNSLRAEDTAVYYCAR | 602 |
| | HFRW4 | WGQGTLVTVSS | 603 |
| | Light Chain | EIVLTQSPATLSLSPGERATLSCRASQSVSSYLAWYQQKPGQAPRLLIY | 604 |
| | | DASNRATGIPARFSGSGSGTDFTLTISSLEPEDFAVYYCQQRSNWPPIT | |
| | | FGPGTKVDIKRTVAAPSVFIFPPSDEQLKSGTASVVCLLNNFYPREAK | |
| | | VQWKVDNALQSGNSQESVTEQDSKDSTYSL | |
| | Light Chain | EIVLTQSPATLSLSPGERATLSCRASQSVSSYLAWYQQKPGQAPRLLIY | 605 |
| | Variable | DASNRATGIPARFSGSGSGTDFTLTISSLEPEDFAVYYCQQRSNWPPIT | |
| | Region | FGPGTKVDIK | |
| | LCDR1 | RASQSVSSYLA | 606 |
| | LCDR2 | DASNRAT | 607 |
| | LCDR3 | QQRSNWPPIT | 608 |
| | LFRW1 | EIVLTQSPATLSLSPGERATLSC | 609 |
| | LFRW2 | WYQQKPGQAPRLLIY | 610 |
| | LFRW3 | GIPARFSGSGSGTDFTLTISSLEPEDFAVYYC | 611 |
| | LFRW4 | FGPGTKVDIK | 612 |
| S24-1002 | Heavy Chain | QVQLVESGGGVVQPGRSLRLSCAASGFTFTSYAMHWVRQAPGKGLE | 613 |
| (Spike/ | | WVAVISYDGGSKYYADSVKGRFTISRDNSKNTLYLQMNSLRAEDTA | |
| RBD) | | VYYCARTTPGITAAGTGTLGRYYYYGMDVWGQGTTVTVSSGSASAP | |
| | | TLFPLVSCENSPSDTSSV | |
| | Heavy Chain | QVQLVESGGGVVQPGRSLRLSCAASGFTFTSYAMHWVRQAPGKGLE | 614 |
| | Variable | WVAVISYDGGSKYYADSVKGRFTISRDNSKNTLYLQMNSLRAEDTA | |
| | Region | VYYCARTTPGITAAGTGTLGRYYYYGMDVWGQGTTVTVSS | |
| | HCDR1 | SYAMH | 615 |
| | HCDR2 | VISYDGGSKYYADSVKG | 616 |
| | HCDR3 | TTPGITAAGTGTLGRYYYYGMDV | 617 |
| | HFRW1 | QVQLVESGGGVVQPGRSLRLSCAASGFTFT | 618 |
| | HFRW2 | WVRQAPGKGLEWVA | 619 |
| | HFRW3 | RFTISRDNSKNTLYLQMNSLRAEDTAVYYCAR | 620 |
| | HFRW4 | WGQGTTVTVSS | 621 |
| | Light Chain | AIQLTQSPSSLSASVGDRVTITCRASQGISSALAWYQQTPGKAPKLLIY | 622 |
| | | DASSLESGVPSRFSGSGSGTDFSLTIGSLQPEDFASYYCQQFNSYPLTF | |
| | | GGGTKVEIKRTVAAPSVFIFPPSDEQLKSGTASVVCLLNNFYPREAKV | |
| | | QWKVDNALQSGNSQESVTEQDSKDSTYSLSSTLTLSKADYE | |
| | Light Chain | AIQLTQSPSSLSASVGDRVTITCRASQGISSALAWYQQTPGKAPKLLIY | 623 |
| | Variable | DASSLESGVPSRFSGSGSGTDFSLTIGSLQPEDFASYYCQQFNSYPLTF | |
| | Region | GGGTKVEIK | |
| | LCDR1 | RASQGISSALA | 624 |
| | LCDR2 | DASSLES | 625 |
| | LCDR3 | QQFNSYPLT | 626 |
| | LFRW1 | AIQLTQSPSSLSASVGDRVTITC | 627 |
| | LFRW2 | WYQQTPGKAPKLLIY | 628 |
| | LFRW3 | GVPSRFSGSGSGTDFSLTIGSLQPEDFASYYC | 629 |
| | LFRW4 | FGGGTKVEIK | 630 |
| S24-1301 | Heavy Chain | QVQLVQSGAEVKKPGASVKVSCKVSGYTLIELSMHWVRQAPGKGLE | 631 |
| (Spike) | | WMGGFDPEDGETIYAQKFQGRVTMTEDTSTDTAYMALSSLTSEDTA | |
| | | VYYCATAYAYYYASGGYYTLDYWGQGTLVTVSSASTKGPSVFPLAP | |
| | | SSKSTSGGTAALGCLVKDYFPEPVTVSWNSGALTSGVHTFPAVLQSS | |
| | | G | |
| | Heavy Chain | QVQLVQSGAEVKKPGASVKVSCKVSGYTLIELSMHWVRQAPGKGLE | 632 |
| | Variable | WMGGFDPEDGETIYAQKFQGRVTMTEDTSTDTAYMALSSLTSEDTA | |
| | Region | VYYCATAYAYYYASGGYYTLDYWGQGTLVTVSS | |
| | HCDR1 | ELSMH | 633 |
| | HCDR2 | GFDPEDGETIYAQKFQG | 634 |
| | HCDR3 | AYAYYYASGGYYTLDY | 635 |
| | HFRW1 | QVQLVQSGAEVKKPGASVKVSCKVSGYTLI | 636 |
| | HFRW2 | WVRQAPGKGLEWMG | 637 |
| | HFRW3 | RVTMTEDTSTDTAYMALSSLTSEDTAVYYCAT | 638 |
| | HFRW4 | WGQGTLVTVSS | 639 |
| | Light Chain | QAGLTQPPSVSKGLRQTATLTCTGSSNNVGNQGAAWLQQHQGHPPK | 640 |
| | | LLSYRNNNRPSGISERFSASRSGNTASLTITGLQPEDEADYYCSAWDSS | |
| | | LSNWVFGGGTKLTVLGQPKAAPSVTLFPPSSEELQANKATLVCLISDF | |
| | | YPGAVTVAWKADSSPVKAGVETTTPSKQSNNKYAASS | |
| | Light Chain | QAGLTQPPSVSKGLRQTATLTCTGSSNNVGNQGAAWLQQHQGHPPK | 641 |
| | Variable | LLSYRNNNRPSGISERFSASRSGNTASLTITGLQPEDEADYYCSAWDSS | |
| | Region | LSNWVFGGGTKLTVL | |
| | LCDR1 | TGSSNNVGNQGAA | 642 |
| | LCDR2 | RNNNRPS | 643 |
| | LCDR3 | SAWDSSLSNWV | 644 |
| | LFRW1 | QAGLTQPPSVSKGLRQTATLTC | 645 |
| | LFRW2 | WLQQHQGHPPKLLSY | 646 |
| | LFRW3 | GISERFSASRSGNTASLTITGLQPEDEADYYC | 647 |
| | LFRW4 | FGGGTKLTVL | 648 |
| S24-223 | Heavy Chain | QITLKESGPTLVKPTQTLTLTCTFSGFSLNTSGVGVGWIRQPPGKALE | 649 |
| (Spike/ | | WLALIYWDDDKRYSPSLKSRLTITKDTSKNQVVLTMTNMDPVDTAT | |
| RBD) | | YYCAHHTIVPIFDYWGQGTLVTVSSGSASAPTLFPLVSCENSPSDTSSV | |
| | Heavy Chain | QITLKESGPTLVKPTQTLTLTCTFSGFSLNTSGVGVGWIRQPPGKALE | 650 |
| | Variable | WLALIYWDDDKRYSPSLKSRLTITKDTSKNQVVLTMTNMDPVDTAT | |
| | Region | YYCAHHTIVPIFDYWGQGTLVTVSS | |
| | HCDR1 | TSGVGVG | 651 |
| | HCDR2 | LIYWDDDKRYSPSLKS | 652 |
| | HCDR3 | HTIVPIFDY | 653 |
| | HFRW1 | QITLKESGPTLVKPTQTLTLTCTFSGFSLN | 654 |
| | HFRW2 | WIRQPPGKALEWLA | 655 |
| | HFRW3 | RLTITKDTSKNQVVLTMTNMDPVDTATYYCAH | 656 |
| | HFRW4 | WGQGTLVTVSS | 657 |
| | Light Chain | QSALTQPASVSGSPGQSITISCTGTSSDVGGYNYVSWYQQHPGKAPKL | 658 |
| | | MIYDVSNRPSGVSNRFSGSKSGNTASLTISGLQAEDEADYYCNSYTSS | |
| | | STLVVFGGGTKLTVLGQPKAAPSVTLFPPSSEELQANKATLVCLISDF | |
| | | YPGAVTVAWKADSSPVKAGVETTTPSKQSNNKYAASSYLSLT | |
| | Light Chain | QSALTQPASVSGSPGQSITISCTGTSSDVGGYNYVSWYQQHPGKAPKL | 659 |
| | Variable | MIYDVSNRPSGVSNRFSGSKSGNTASLTISGLQAEDEADYYCNSYTSS | |
| | Region | STLVVFGGGTKLTVL | |
| | LCDR1 | TGTSSDVGGYNYVS | 660 |
| | LCDR2 | DVSNRPS | 661 |
| | LCDR3 | NSYTSSSTLVV | 662 |
| | LFRW1 | QSALTQPASVSGSPGQSITISC | 663 |
| | LFRW2 | WYQQHPGKAPKLMIY | 664 |
| | LFRW3 | GVSNRFSGSKSGNTASLTISGLQAEDEADYYC | 665 |
| | LFRW4 | FGGGTKLTVL | 666 |
| S24-461 | Heavy Chain | QVQLQESGPGLVKPSETLSLTCTVSGGSISSYYWSWIRQPPGKGLEWI | 667 |
| (Spike/ | | GNIYNSGSTNYNPSLKSRLTISVDTSKNHFSLKLSSVTAADTAVYYCA | |
| RBD) | | RGGLEHDGDYVYYYGMDVWGQGTTITVSSASTKGPSVFPLAPSSKST | |
| | | SGGTAALGCLVKDYFPEPVTVSWNSGALTSGVHTFPAVLQSSG | |
| | Heavy Chain | QVQLQESGPGLVKPSETLSLTCTVSGGSISSYYWSWIRQPPGKGLEWI | 668 |
| | Variable | GNIYNSGSTNYNPSLKSRLTISVDTSKNHFSLKLSSVTAADTAVYYCA | |
| | Region | RGGLEHDGDYVYYYGMDVWGQGTTITVSS | |
| | HCDR1 | SYYWS | 669 |
| | HCDR2 | NIYNSGSTNYNPSLKS | 670 |
| | HCDR3 | GGLEHDGDYVYYYGMDV | 671 |
| | HFRW1 | QVQLQESGPGLVKPSETLSLTCTVSGGSIS | 672 |
| | HFRW2 | WIRQPPGKGLEWIG | 673 |
| | HFRW3 | RLTISVDTSKNHFSLKLSSVTAADTAVYYCAR | 674 |
| | HFRW4 | WGQGTTITVSS | 675 |
| | Light Chain | SYELTQPPSVSVSLGQMARITCSGEALPKKYAYWYQQKPGQFPILVIY | 676 |
| | | KDSERPSGIPERFSGSSSGTIVTLTISGVQAEDEADYYCLSEDSSGTWV | |
| | | FGGGTKLTVLGQPKAAPSVTLFPPSSEELQANKATLVCLISDFYPGAV | |
| | | TVAWKADSSPVKAGVETTTPSKQSNNKYAASS | |
| | Light Chain | SYELTQPPSVSVSLGQMARITCSGEALPKKYAYWYQQKPGQFPILVIY | 677 |
| | Variable | KDSERPSGIPERFSGSSSGTIVTLTISGVQAEDEADYYCLSEDSSGTWV | |
| | Region | FGGGTKLTVL | |
| | LCDR1 | SGEALPKKYAY | 678 |
| | LCDR2 | KDSERPS | 679 |
| | LCDR3 | LSEDSSGTWV | 680 |
| | LFRW1 | SYELTQPPSVSVSLGQMARITC | 681 |
| | LFRW2 | WYQQKPGQFPILVIY | 682 |
| | LFRW3 | GIPERFSGSSSGTIVTLTISGVQAEDEADYYC | 683 |
| | LFRW4 | FGGGTKLTVL | 684 |
| S24-511 | Heavy Chain | QVQLVESGGGVVQPGRSLRLSCAASGFTFSSYGMHWVRQAPGKGLE | 685 |
| (NP) | | WVAVISYDGSNKYYADSVKGRFTISRDNSKNTLYLQMNSLRAEDTA | |
| | | VYYCAKYTSTVTTNYYYGMDVWGQGTTVTVSSAPTKAPDVFPIISGC | |
| | | RHPKDNSPVVLACLITGYH | |
| | Heavy Chain | QVQLVESGGGVVQPGRSLRLSCAASGFTFSSYGMHWVRQAPGKGLE | 686 |
| | Variable | WVAVISYDGSNKYYADSVKGRFTISRDNSKNTLYLQMNSLRAEDTA | |
| | Region | VYYCAKYTSTVTTNYYYGMDVWGQGTTVTVSS | |
| | HCDR1 | SYGMH | 687 |
| | HCDR2 | VISYDGSNKYYADSVKG | 688 |
| | HCDR3 | YTSTVTTNYYYGMDV | 689 |
| | HFRW1 | QVQLVESGGGVVQPGRSLRLSCAASGFTFS | 690 |
| | HFRW2 | WVRQAPGKGLEWVA | 691 |
| | HFRW3 | RFTISRDNSKNTLYLQMNSLRAEDTAVYYCAK | 692 |
| | HFRW4 | WGQGTTVTVSS | 693 |
| | Light Chain | SYELTQPPSVSVSPGQTASITCSGDKLGDKYACWYQQKPGQSPVLVIY | 694 |
| | | QDSKRPSGIPERFSGSNSGNTATLTISGTQAMDEADYYCQAWDSSTV | |
| | | VFGGGTKLTVLGQPKAAPSVTLFPPSSEELQANKATLVCLISDFYPGA | |
| | | VTVAWKADSSPVKAGVETTTPSKQSNNKYAASSY | |
| | Light Chain | SYELTQPPSVSVSPGQTASITCSGDKLGDKYACWYQQKPGQSPVLVIY | 695 |
| | Variable | QDSKRPSGIPERFSGSNSGNTATLTISGTQAMDEADYYCQAWDSSTV | |
| | Region | VFGGGTKLTVL | |
| | LCDR1 | SGDKLGDKYAC | 696 |
| | LCDR2 | QDSKRPS | 697 |
| | LCDR3 | QAWDSSTVV | 698 |
| | LFRW1 | SYELTQPPSVSVSPGQTASITC | 699 |
| | LFRW2 | WYQQKPGQSPVLVIY | 700 |
| | LFRW3 | GIPERFSGSNSGNTATLTISGTQAMDEADYYC | 701 |
| | LFRW4 | FGGGTKLTVL | 702 |
| S24-788 | Heavy Chain | QVQLVESGGGVVQPGRSLRLSCAASGFTFSSYGMHWVRQAPGKGLE | 703 |
| (Spike/ | | WVAVIWYDGSNKYYADSVKGRFTISRDNSKNTLYLQMNSLRAEDTA | |
| RBD) | | VYYCARGRSPGGGHYYGMDVWGQGTTVTVSSGSASAPTLFPLVSCE | |
| | | NSPSDTSSV | |
| | Heavy Chain | QVQLVESGGGVVQPGRSLRLSCAASGFTFSSYGMHWVRQAPGKGLE | 704 |
| | Variable | WVAVIWYDGSNKYYADSVKGRFTISRDNSKNTLYLQMNSLRAEDTA | |
| | Region | VYYCARGRSPGGGHYYGMDVWGQGTTVTVSS | |
| | HCDR1 | SYGMH | 705 |
| | HCDR2 | VIWYDGSNKYYADSVKG | 706 |
| | HCDR3 | GRSPGGGHYYGMDV | 707 |
| | HFRW1 | QVQLVESGGGVVQPGRSLRLSCAASGFTFS | 708 |
| | HFRW2 | WVRQAPGKGLEWVA | 709 |
| | HFRW3 | RFTISRDNSKNTLYLQMNSLRAEDTAVYYCAR | 710 |
| | HFRW4 | WGQGTTVTVSS | 711 |
| | Light Chain | SYELTQPPSVSVSPGQTASITCSGDKLGDKYACWYQQKPGQSPVLVIY | 712 |
| | | QDSKRPSGIPERFSGSNSGNTATLTISGTQAMDEADYYCQAWDSSSV | |
| | | VFGGGTKLTVLGQPKAAPSVTLFPPSSEELQANKATLVCLISDFYPGA | |
| | | VTVAWKADSSPVKAGVETTTPSKQSNNKYAASS | |
| | Light Chain | SYELTQPPSVSVSPGQTASITCSGDKLGDKYACWYQQKPGQSPVLVIY | 713 |
| | Variable | QDSKRPSGIPERFSGSNSGNTATLTISGTQAMDEADYYCQAWDSSSV | |
| | Region | VFGGGTKLTVL | |
| | LCDR1 | SGDKLGDKYAC | 714 |
| | LCDR2 | QDSKRPS | 715 |
| | LCDR3 | QAWDSSSVV | 716 |
| | LFRW1 | SYELTQPPSVSVSPGQTASITC | 717 |
| | LFRW2 | WYQQKPGQSPVLVIY | 718 |
| | LFRW3 | GIPERFSGSNSGNTATLTISGTQAMDEADYYC | 719 |
| | LFRW4 | FGGGTKLTVL | 720 |
| S24-821 | Heavy Chain | QVTLRESGPALVKPTQTLTLTCTFSGLSLSSSGMCVSWIRQPPGKALE | 721 |
| (Spike/ | | WLARIDWDDDKYYSTSLKTRLTISKDTSKNQVVLTMTNMDPVDTAT | |
| RBD) | | YYCARICTMVRGLHDAFDIWGQGTMVTVSSGSASAPTLFPLVSCENS | |
| | | PSDTSSV | |
| | Heavy Chain | QVTLRESGPALVKPTQTLTLTCTFSGLSLSSSGMCVSWIRQPPGKALE | 722 |
| | Variable | WLARIDWDDDKYYSTSLKTRLTISKDTSKNQVVLTMTNMDPVDTAT | |
| | Region | YYCARICTMVRGLHDAFDIWGQGTMVTVSS | |
| | HCDR1 | SSGMCVS | 723 |
| | HCDR2 | RIDWDDDKYYSTSLKT | 724 |
| | HCDR3 | ICTMVRGLHDAFDI | 725 |
| | HFRW1 | QVTLRESGPALVKPTQTLTLTCTFSGLSLS | 726 |
| | HFRW2 | WIRQPPGKALEWLA | 727 |
| | HFRW3 | RLTISKDTSKNQVVLTMTNMDPVDTATYYCAR | 728 |
| | HFRW4 | WGQGTMVTVSS | 729 |
| | Light Chain | DIQMTQSPSTLSASVGDRVTITCRASQSISSWLAWYQQKPGKAPKLLI | 730 |
| | | YKASSLESGVPSRFSGSGSGTEFTLTISSLQPDDFATYYCQQYNSYSW | |
| | | TFGQGTKVEIKRTVAAPSVFIFPPSDEQLKSGTASVVCLLNNFYPREA | |
| | | KVQWKVDN | |
| | Light Chain | DIQMTQSPSTLSASVGDRVTITCRASQSISSWLAWYQQKPGKAPKLLI | 731 |
| | Variable | YKASSLESGVPSRFSGSGSGTEFTLTISSLQPDDFATYYCQQYNSYSW | |
| | Region | TFGQGTKVEIK | |
| | LCDR1 | RASQSISSWLA | 732 |
| | LCDR2 | KASSLES | 733 |
| | LCDR3 | QQYNSYSWT | 734 |
| | LFRW1 | DIQMTQSPSTLSASVGDRVTITC | 735 |
| | LFRW2 | WYQQKPGKAPKLLIY | 736 |
| | LFRW3 | GVPSRFSGSGSGTEFTLTISSLQPDDFATYYC | 737 |
| | LFRW4 | FGQGTKVEIK | 738 |
| S144-67 | Heavy Chain | EVQLVQSGAEVKKPGESLKISCKGSGYSFTTYWIAWVRQMPGKGLE | 739 |
| (Spike/ | | WVGIIYPDDSDTRYSPSFQGQVTISADKSIGTAYLQWSSLKASDTAMY | |
| RBD) | | YCARGQYYDFWSGAGGVDVWGQGTTVTVSSASTKGPSVFPLAPSSK | |
| | | STSGGTAALGCLVKDYFPEPVTVSWNSGALTSGVHTFPAVLQSSG | |
| | Heavy Chain | EVQLVQSGAEVKKPGESLKISCKGSGYSFTTYWIAWVRQMPGKGLE | 740 |
| | Variable | WVGIIYPDDSDTRYSPSFQGQVTISADKSIGTAYLQWSSLKASDTAMY | |
| | Region | YCARGQYYDFWSGAGGVDVWGQGTTVTVSS | |
| | HCDR1 | TYWIA | 741 |
| | HCDR2 | IIYPDDSDTRYSPSFQG | 742 |
| | HCDR3 | GQYYDFWSGAGGVDV | 743 |
| | HFRW1 | EVQLVQSGAEVKKPGESLKISCKGSGYSFT | 744 |
| | HFRW2 | WVRQMPGKGLEWVG | 745 |
| | HFRW3 | QVTISADKSIGTAYLQWSSLKASDTAMYYCAR | 746 |
| | HFRW4 | WGQGTTVTVSS | 747 |
| | Light Chain | QSVLTQPPSVSGAPGQRVTISCTGSRSNIGAGYDVQWYQQVPGTAPK | 748 |
| | | LLISGNSNRPSGVPDRFSGSKSGTSASLAITGLQAEDEADYYCQSYDSS | |
| | | LSGLRVFGGGTKLTVLGQPKAAPSVTLFPPSSEELQANKATLVCLISD | |
| | | FYPGAVTVAWKADSSPVKAGVETTTPSKQSNNKYAASSYLSLTPEQ | |
| | | WKSH | |
| | Light Chain | QSVLTQPPSVSGAPGQRVTISCTGSRSNIGAGYDVQWYQQVPGTAPK | 749 |
| | Variable | LLISGNSNRPSGVPDRFSGSKSGTSASLAITGLQAEDEADYYCQSYDSS | |
| | Region | LSGLRVFGGGTKLTVL | |
| | LCDR1 | TGSRSNIGAGYDVQ | 750 |
| | LCDR2 | GNSNRPS | 751 |
| | LCDR3 | QSYDSSLSGLRV | 752 |
| | LFRW1 | QSVLTQPPSVSGAPGQRVTISC | 753 |
| | LFRW2 | WYQQVPGTAPKLLIS | 754 |
| | LFRW3 | GVPDRFSGSKSGTSASLAITGLQAEDEADYYC | 755 |
| | LFRW4 | FGGGTKLTVL | 756 |
| S144-69 | Heavy Chain | EVQLVQSGAEVKKPGESLKISCKGSGYSFTSYWIGWVRQMPGKGLE | 757 |
| (Spike/ | | WMGIIYPGDSDTRYSPSFQGQVTISADKSITTAYLQWSSLKASDTAMY | |
| RBD) | | YCARTQTTNWFDSWGQGTLVTVSSASTKGPSVFPLAPSSKSTSGGTA | |
| | | ALGCLVKDYFPEPVTVSWNSGALTSGVHTFPAVLQSSG | |
| | Heavy Chain | EVQLVQSGAEVKKPGESLKISCKGSGYSFTSYWIGWVRQMPGKGLE | 758 |
| | Variable | WMGIIYPGDSDTRYSPSFQGQVTISADKSITTAYLQWSSLKASDTAMY | |
| | Region | YCARTQTTNWFDSWGQGTLVTVSS | |
| | HCDR1 | SYWIG | 759 |
| | HCDR2 | IIYPGDSDTRYSPSFQG | 760 |
| | HCDR3 | TQTTNWFDS | 761 |
| | HFRW1 | EVQLVQSGAEVKKPGESLKISCKGSGYSFT | 762 |
| | HFRW2 | WVRQMPGKGLEWMG | 763 |
| | HFRW3 | QVTISADKSITTAYLQWSSLKASDTAMYYCAR | 764 |
| | HFRW4 | WGQGTLVTVSS | 765 |
| | Light Chain | DIQMTQSPSTLSVSVGDRVTITCRASQSVSSWLAWYQQKPGKAPKLLI | 766 |
| | | YDASSLESGVPSRFSGSGSGTEFTLTISSLQPDDFATYYCQQYNSFYTF | |
| | | GQGTKLEIKRTVAAPSVFIFPPSDEQLKSGTASVVCLLNNFYPREAKV | |
| | | QWKVDNALQSGNSQESVTEQDSKDSTYSLSSTLTLSKADYE | |
| | Light Chain | DIQMTQSPSTLSVSVGDRVTITCRASQSVSSWLAWYQQKPGKAPKLLI | 767 |
| | Variable | YDASSLESGVPSRFSGSGSGTEFTLTISSLQPDDFATYYCQQYNSFYTF | |
| | Region | GQGTKLEIK | |
| | LCDR1 | RASQSVSSWLA | 768 |
| | LCDR2 | DASSLES | 769 |
| | LCDR3 | QQYNSFYT | 770 |
| | LFRW1 | DIQMTQSPSTLSVSVGDRVTITC | 771 |
| | LFRW2 | WYQQKPGKAPKLLIY | 772 |
| | LFRW3 | GVPSRFSGSGSGTEFTLTISSLQPDDFATYYC | 773 |
| | LFRW4 | FGQGTKLEIK | 774 |
| S144-94 | Heavy Chain | QVQLVESGGGVVQPGGSLRLSCAASGFTFSSYGMHWVRQAPGKGLE | 775 |
| (ORF8) | | WVTFTRYDGSNKFYADSVKGRFSISRDNSKNTLYLQMNSLRAEDTA | |
| | | VYYCAKESRVAFGGAIAIYYFGMDVWGQGTTVTVSSASTKGPSVFPL | |
| | | APCSRSTSGGTAALGCLVKDYFPEPVTVSWNSGALTSGVHTFPAVLQ | |
| | | SSG | |
| | Heavy Chain | QVQLVESGGGVVQPGGSLRLSCAASGFTFSSYGMHWVRQAPGKGLE | 776 |
| | Variable | WVTFTRYDGSNKFYADSVKGRFSISRDNSKNTLYLQMNSLRAEDTA | |
| | Region | VYYCAKESRVAFGGAIAIYYFGMDVWGQGTTVTVSS | |
| | HCDR1 | SYGMH | 777 |
| | HCDR2 | FTRYDGSNKFYADSVKG | 778 |
| | HCDR3 | ESRVAFGGAIAIYYFGMDV | 779 |
| | HFRW1 | QVQLVESGGGVVQPGGSLRLSCAASGFTFS | 780 |
| | HFRW2 | WVRQAPGKGLEWVT | 781 |
| | HFRW3 | RFSISRDNSKNTLYLQMNSLRAEDTAVYYCAK | 782 |
| | HFRW4 | WGQGTTVTVSS | 783 |
| | Light Chain | DIVMTQSPLSLPVTPGEPASISCRSSQSLLHSNGYNYLDWYLQKPGQS | 784 |
| | | PQLLIYLGSNRASGVPDRFSGSGSGTDFTLKISRVEAEDVGVYYCMQA | |
| | | LQTPQYTFGQGTKLEIKRTVAAPSVFIFPPSDEQLKSGTASVVCLLNNF | |
| | | YPREAKVQWKVDNALQSGNSQESVTEQDSKDSTYSLSSTLTLSKADY | |
| | | E | |
| | Light Chain | DIVMTQSPLSLPVTPGEPASISCRSSQSLLHSNGYNYLDWYLQKPGQS | 785 |
| | Variable | PQLLIYLGSNRASGVPDRFSGSGSGTDFTLKISRVEAEDVGVYYCMQA | |
| | Region | LQTPQYTFGQGTKLEIK | |
| | LCDR1 | RSSQSLLHSNGYNYLD | 786 |
| | LCDR2 | LGSNRAS | 787 |
| | LCDR3 | MQALQTPQYT | 788 |
| | LFRW1 | DIVMTQSPLSLPVTPGEPASISC | 789 |
| | LFRW2 | WYLQKPGQSPQLLIY | 790 |
| | LFRW3 | GVPDRFSGSGSGTDFTLKISRVEAEDVGVYYC | 791 |
| | LFRW4 | FGQGTKLEIK | 792 |
| S144-113 | Heavy Chain | EVQLLESGGGLVQPGGSLRLSCAASGFTFSNYAMSWVRQAPGKGLE | 793 |
| (ORF8) | | WVSAIRNSGSSTYYADSVKGRFTISRDNSKNTLYLQMNSLRAEDSAV | |
| | | YYCAKVGGTAAGHPFYDYWGQGTLVTVSSASTKGPSVFPLAPSSKST | |
| | | SGGTAALGCLVKDYFPEPVTVSWNSGALTSGVHTFPAVLQSSGL | |
| | Heavy Chain | EVQLLESGGGLVQPGGSLRLSCAASGFTFSNYAMSWVRQAPGKGLE | 794 |
| | Variable | WVSAIRNSGSSTYYADSVKGRFTISRDNSKNTLYLQMNSLRAEDSAV | |
| | Region | YYCAKVGGTAAGHPFYDYWGQGTLVTVSS | |
| | HCDR1 | NYAMS | 795 |
| | HCDR2 | AIRNSGSSTYYADSVKG | 796 |
| | HCDR3 | VGGTAAGHPFYDY | 797 |
| | HFRW1 | EVQLLESGGGLVQPGGSLRLSCAASGFTFS | 798 |
| | HFRW2 | WVRQAPGKGLEWVS | 799 |
| | HFRW3 | RFTISRDNSKNTLYLQMNSLRAEDSAVYYCAK | 800 |
| | HFRW4 | WGQGTLVTVSS | 801 |
| | Light Chain | DIQMTQSPSSLSASVGDRVTITCRASQSISNYLNWYQQKPGKAPDLLI | 802 |
| | | YAASSLQSGVPLRFSGSGSGTDFTLTISSLQPEDFATYYCQQTYSAPTF | |
| | | GGGTKVEIKRTVAAPSVFIFPPSDEQLKSGTASVVCLLNNFYPREAKV | |
| | | QWKVDNALQSGNSQESVTEQDSKDSTYSLSSTLTLSKADYE | |
| | Light Chain | DIQMTQSPSSLSASVGDRVTITCRASQSISNYLNWYQQKPGKAPDLLI | 803 |
| | Variable | YAASSLQSGVPLRFSGSGSGTDFTLTISSLQPEDFATYYCQQTYSAPTF | |
| | Region | GGGTKVEIK | |
| | LCDR1 | RASQSISNYLN | 804 |
| | LCDR2 | AASSLQS | 805 |
| | LCDR3 | QQTYSAPT | 806 |
| | LFRW1 | DIQMTQSPSSLSASVGDRVTITC | 807 |
| | LFRW2 | WYQQKPGKAPDLLIY | 808 |
| | LFRW3 | GVPLRFSGSGSGTDFTLTISSLQPEDFATYYC | 809 |
| | LFRW4 | FGGGTKVEIK | 810 |
| S144-175 | Heavy Chain | QVQLVQSGAEVKKPGASVKVSCKASGYTFTGYYMHWVRQAPGQGL | 811 |
| (ORF8) | | EWMGRINPNSGGTNFAQRFQGRVSMTRDTSISTAYMELSSLRSDDTA | |
| | | VYYCARGAKFEHLPFDIWGQGTMVTVSSASTKGPSVFPLAPSSKSTSG | |
| | | GTAALGCLVKDYFPEPVTVSWNSGALTSGVHTFPAVLQSSG | |
| | Heavy Chain | QVQLVQSGAEVKKPGASVKVSCKASGYTFTGYYMHWVRQAPGQGL | 812 |
| | Variable | EWMGRINPNSGGTNFAQRFQGRVSMTRDTSISTAYMELSSLRSDDTA | |
| | Region | VYYCARGAKFEHLPFDIWGQGTMVTVSS | |
| | HCDR1 | GYYMH | 813 |
| | HCDR2 | RINPNSGGTNFAQRFQG | 814 |
| | HCDR3 | GAKFEHLPFDI | 815 |
| | HFRW1 | QVQLVQSGAEVKKPGASVKVSCKASGYTFT | 816 |
| | HFRW2 | WVRQAPGQGLEWMG | 817 |
| | HFRW3 | RVSMTRDTSISTAYMELSSLRSDDTAVYYCAR | 818 |
| | HFRW4 | WGQGTMVTVSS | 819 |
| | Light Chain | QSMLTQPPSASGTPGQRVTISCSGSSSNIGSNYVYWYQQLPGTAPKLLI | 820 |
| | | YRNNQRPSGVPDRFSGSKSGTSASLAISGLRSEDEADYYCAAWDDRR | |
| | | WVFGGGTKLTVLGQPKAAPSVTLFPPSSEELQANKATLVCLISDFYPG | |
| | | AVTVAWKADSSPVKAGVETTTPSKQSNNKYAASS | |
| | Light Chain | QSMLTQPPSASGTPGQRVTISCSGSSSNIGSNYVYWYQQLPGTAPKLLI | 821 |
| | Variable | YRNNQRPSGVPDRFSGSKSGTSASLAISGLRSEDEADYYCAAWDDRR | |
| | Region | WVFGGGTKLTVL | |
| | LCDR1 | SGSSSNIGSNYVY | 822 |
| | LCDR2 | RNNQRPS | 823 |
| | LCDR3 | AAWDDRRWV | 824 |
| | LFRW1 | QSMLTQPPSASGTPGQRVTISC | 825 |
| | LFRW2 | WYQQLPGTAPKLLIY | 826 |
| | LFRW3 | GVPDRFSGSKSGTSASLAISGLRSEDEADYYC | 827 |
| | LFRW4 | FGGGTKLTVL | 828 |
| S144-208 | Heavy Chain | QVQLVQSGAEVKKPGASVKVSCKSSGYTFTGYYMHWVRQAPGQGL | 829 |
| (ORF8) | | EWMGRINPNSGGTNYAQKFQGRVTMTRDTSISTAYMELSRLRSDDT | |
| | | AVYYCARGARGGAGCSGWSCFDFWGQGTLVTVSSASTKGPSVFPLA | |
| | | PSSKSTSGGTAALGCLVKDYFPEPVTVSWNSGALTSGVHTFPAVLQSS | |
| | | G | |
| | Heavy Chain | QVQLVQSGAEVKKPGASVKVSCKSSGYTFTGYYMHWVRQAPGQGL | 830 |
| | Variable | EWMGRINPNSGGTNYAQKFQGRVTMTRDTSISTAYMELSRLRSDDT | |
| | Region | AVYYCARGARGGAGCSGWSCFDFWGQGTLVTVSS | |
| | HCDR1 | GYYMH | 831 |
| | HCDR2 | RINPNSGGTNYAQKFQG | 832 |
| | HCDR3 | GARGGAGCSGWSCFDF | 833 |
| | HFRW1 | QVQLVQSGAEVKKPGASVKVSCKSSGYTFT | 834 |
| | HFRW2 | WVRQAPGQGLEWMG | 835 |
| | HFRW3 | RVTMTRDTSISTAYMELSRLRSDDTAVYYCAR | 836 |
| | HFRW4 | WGQGTLVTVSS | 837 |
| | Light Chain | QSALTQPRSVSGSPGQSVTISCTGTSSDVGGYKYVSWYQQHPGKAPK | 838 |
| | | LMIYDVSKRPSGVPDRFSGSKSGNTASLTISGLQAEDEGDYYCCSYAG | |
| | | TYSLVFGGGTKVTVTVLGQPKAAPSVTLFPPSSEELQANKATLVCLIS | |
| | | DFYPGAVTVAWKADSSPVKAGVETTTPSKQSNNKYAASSYLSLTPEQ | |
| | | WKSH | |
| | Light Chain | QSALTQPRSVSGSPGQSVTISCTGTSSDVGGYKYVSWYQQHPGKAPK | 839 |
| | Variable | LMIYDVSKRPSGVPDRFSGSKSGNTASLTISGLQAEDEGDYYCCSYAG | |
| | Region | TYSLVFGGGTKVTV | |
| | LCDR1 | TGTSSDVGGYKYVS | 840 |
| | LCDR2 | DVSKRPS | 841 |
| | LCDR3 | CSYAGTYSLV | 842 |
| | LFRW1 | QSALTQPRSVSGSPGQSVTISC | 843 |
| | LFRW2 | WYQQHPGKAPKLMIY | 844 |
| | LFRW3 | GVPDRFSGSKSGNTASLTISGLQAEDEGDYYC | 845 |
| | LFRW4 | FGGGTKVTV | 846 |
| S144-339 | Heavy Chain | EVQLVESGGGLVKPGGSLRLSCAASGFTFSDYTMNWVRQAPGKGLE | |
| (NP) | | WVSSITRSSTYIYYADSVKGRFTISRDNAKNSLYLQMNSLRAEDTAVY | |
| | | YCARDPYYDILTGYWNYWGQGTLVTVSSASTKGPSVFPLAPSSKSTS | |
| | | GGTAALGCLVKDYFPEPVTVSWNSGALTSGVHTFPAVLQSSG | 847 |
| | Heavy Chain | EVQLVESGGGLVKPGGSLRLSCAASGFTFSDYTMNWVRQAPGKGLE | 848 |
| | Variable | WVSSITRSSTYIYYADSVKGRFTISRDNAKNSLYLQMNSLRAEDTAVY | |
| | Region | YCARDPYYDILTGYWNYWGQGTLVTVSS | |
| | HCDR1 | DYTMN | 849 |
| | HCDR2 | SITRSSTYIYYADSVKG | 850 |
| | HCDR3 | DPYYDILTGYWNY | 851 |
| | HFRW1 | EVQLVESGGGLVKPGGSLRLSCAASGFTFS | 852 |
| | HFRW2 | WVRQAPGKGLEWVS | 853 |
| | HFRW3 | RFTISRDNAKNSLYLQMNSLRAEDTAVYYCAR | 854 |
| | HFRW4 | WGQGTLVTVSS | 855 |
| | Light Chain | EIVLTQSPGTLSLSPGERATLSCRASQSLSSSYLAWYQQKPGQSPRLLI | 856 |
| | | YGASSRATGIPDRFSGSGSGTDFTLTINRLEPEDFAVYYCQQYRTSPRG | |
| | | TFGGGTKVEIKRTVAAPSVFIFPPSDEQLKSGTASVVCLLNNFYPREA | |
| | | KVQWKVDNALQSGNSQESVTEQDSKDSTYSLSSTLTLSKADYE | |
| | Light Chain | EIVLTQSPGTLSLSPGERATLSCRASQSLSSSYLAWYQQKPGQSPRLLI | 857 |
| | Variable | YGASSRATGIPDRFSGSGSGTDFTLTINRLEPEDFAVYYCQQYRTSPRG | |
| | Region | TFGGGTKVEIK | |
| | LCDR1 | RASQSLSSSYLA | 858 |
| | LCDR2 | GASSRAT | 859 |
| | LCDR3 | QQYRTSPRGT | 860 |
| | LFRW1 | EIVLTQSPGTLSLSPGERATLSC | 861 |
| | LFRW2 | WYQQKPGQSPRLLIY | 862 |
| | LFRW3 | GIPDRFSGSGSGTDFTLTINRLEPEDFAVYYC | 863 |
| | LFRW4 | FGGGTKVEIK | 864 |
| S144-359 | Heavy Chain | EVQLVESGGGLVQPGGSLRLSCAASGFTFSSYAMSWVRQAPGKGLE | 865 |
| (ORF8) | | WVSSIRGSGGSTYYADSVKGRFTISRDNSKYTLYLQMNSLRAEDTAV | |
| | | YYCAKITGAVGGENWFDPWGQGTLVTVSSASTKGPSVFPLAPCSRST | |
| | | SGGTAALGCLVKDYFPEPVTVSWNSGALTSGVHTFPAVLQSSG | |
| | Heavy Chain | EVQLVESGGGLVQPGGSLRLSCAASGFTFSSYAMSWVRQAPGKGLE | 866 |
| | Variable | WVSSIRGSGGSTYYADSVKGRFTISRDNSKYTLYLQMNSLRAEDTAV | |
| | Region | YYCAKITGAVGGENWFDPWGQGTLVTVSS | |
| | HCDR1 | SYAMS | 867 |
| | HCDR2 | SIRGSGGSTYYADSVKG | 868 |
| | HCDR3 | ITGAVGGENWFDP | 869 |
| | HFRW1 | EVQLVESGGGLVQPGGSLRLSCAASGFTFS | 870 |
| | HFRW2 | WVRQAPGKGLEWVS | 871 |
| | HFRW3 | RFTISRDNSKYTLYLQMNSLRAEDTAVYYCAK | 872 |
| | HFRW4 | WGQGTLVTVSS | 873 |
| | Light Chain | DIQMTQSPSSLSASVGDRVTITCRASQSISSYLNWYQQKPGKAPKLLIY | 874 |
| | | AASSLQSGVPSRFSGSGSGTDFTLTISSLQPEDFAIYYCQQTSRTPLTFG | |
| | | GGTKVEVKRTVAAPSVFIFPPSDEQLKSGTASVVCLLNNFYPREAKVQ | |
| | | WKVDNALQSGNSQESVTEQDSKDSTYSLSSTLTLSKADYE | |
| | Light Chain | DIQMTQSPSSLSASVGDRVTITCRASQSISSYLNWYQQKPGKAPKLLIY | 875 |
| | Variable | AASSLQSGVPSRFSGSGSGTDFTLTISSLQPEDFAIYYCQQTSRTPLTFG | |
| | Region | GGTKVEVK | |
| | LCDR1 | RASQSISSYLN | 876 |
| | LCDR2 | AASSLQS | 877 |
| | LCDR3 | QQTSRTPLT | 878 |
| | LFRW1 | DIQMTQSPSSLSASVGDRVTITC | 879 |
| | LFRW2 | WYQQKPGKAPKLLIY | 880 |
| | LFRW3 | GVPSRFSGSGSGTDFTLTISSLQPEDFAIYYC | 881 |
| | LFRW4 | FGGGTKVEVK | 882 |
| S144-460 | Heavy Chain | EVRLVQSGGGLVKPGGSLRLSCAASGFTFSTAWVRWVRQAPGKGLE | 883 |
| (Spike/ | | CVGRIKSKNDGDRAEYAAPARGRFIISRDDAENILYLQMNNLKTEDT | |
| RBD) | | AFYYCTTDQGNSSAFYSADYWGQGTLVTVSSASPTSPKVFPLSLDSTP | |
| | | QDGNVVVACLVQGFFPQEPLSVTWSESGQNVTARNF | |
| | Heavy Chain | EVRLVQSGGGLVKPGGSLRLSCAASGFTFSTAWVRWVRQAPGKGLE | 884 |
| | Variable | CVGRIKSKNDGDRAEYAAPARGRFIISRDDAENILYLQMNNLKTEDT | |
| | Region | AFYYCTTDQGNSSAFYSADYWGQGTLVTVSS | |
| | HCDR1 | TAWVR | 885 |
| | HCDR2 | RIKSKNDGDRAEYAAPARG | 886 |
| | HCDR3 | DQGNSSAFYSADY | 887 |
| | HFRW1 | EVRLVQSGGGLVKPGGSLRLSCAASGFTFS | 888 |
| | HFRW2 | WVRQAPGKGLECVG | 889 |
| | HFRW3 | RFIISRDDAENILYLQMNNLKTEDTAFYYCTT | 890 |
| | HFRW4 | WGQGTLVTVSS | 891 |
| | Light Chain | DIQMTQSPSAMSASVGDRVTITCRASQDINTFLTWFQQKPGKVPQRLI | 892 |
| | | FAAYRLQSGVPSRFSGSGSGTEFTLTINSLQPEDVATYYCLHHKTYPY | |
| | | TFGQGTKLEIKRTVAAPSVFIFPPSDEQLKSGTASVVCLLNNFYPREAK | |
| | | VQWKVDNALQSGNSQESVTEQDSKDSTYSLSSTLTLSKADYE | |
| | Light Chain | DIQMTQSPSAMSASVGDRVTITCRASQDINTFLTWFQQKPGKVPQRLI | 893 |
| | Variable | FAAYRLQSGVPSRFSGSGSGTEFTLTINSLQPEDVATYYCLHHKTYPY | |
| | Region | TFGQGTKLEIK | |
| | LCDR1 | RASQDINTFLT | 894 |
| | LCDR2 | AAYRLQS | 895 |
| | LCDR3 | LHHKTYPYT | 896 |
| | LFRW1 | DIQMTQSPSAMSASVGDRVTITC | 897 |
| | LFRW2 | WFQQKPGKVPQRLIF | 898 |
| | LFRW3 | GVPSRFSGSGSGTEFTLTINSLQPEDVATYYC | 899 |
| | LFRW4 | FGQGTKLEIK | 900 |
| S144-466 | Heavy Chain | EVQLVQSGAEVKKPGESLKISCKGSGYRFTRYWIGWVRQMPGKGLE | 901 |
| (Spike/ | | WMGIIYLGDSETRYSPSFQGQVTISADNSISTAYLQWSSLKASDTAMY | |
| RBD) | | YCARSSNWNYGDYWGQGTLVTVSSASTKGPSVFPLAPCSRSTSGGTA | |
| | | ALGCLVKDYFPEPVTVSWNSGALTSGVHTFPAVLQSSG | |
| | Heavy Chain | EVQLVQSGAEVKKPGESLKISCKGSGYRFTRYWIGWVRQMPGKGLE | 902 |
| | Variable | WMGIIYLGDSETRYSPSFQGQVTISADNSISTAYLQWSSLKASDTAMY | |
| | Region | YCARSSNWNYGDYWGQGTLVTVSS | |
| | HCDR1 | RYWIG | 903 |
| | HCDR2 | IIYLGDSETRYSPSFQG | 904 |
| | HCDR3 | SSNWNYGDY | 905 |
| | HFRW1 | EVQLVQSGAEVKKPGESLKISCKGSGYRFT | 906 |
| | HFRW2 | WVRQMPGKGLEWMG | 907 |
| | HFRW3 | QVTISADNSISTAYLQWSSLKASDTAMYYCAR | 908 |
| | HFRW4 | WGQGTLVTVSS | 909 |
| | Light Chain | DIQMTQSPSTLSASVGDRVTITCRASQSITSWLAWYQQKSGKAPKLLI | 910 |
| | | YDASSLESGVPSRFSGSGSGTEFTLTISSLQPDDFATYYCQQYNSYPW | |
| | | TFGQGTKVEIKRTVAAPSVFIFPPSDEQLKSGTASVVCLLNNFYPREA | |
| | | KVQWKVDNALQSGNSQESVTEQDSKDSTYSLSSTLTLSKADYE | |
| | Light Chain | DIQMTQSPSTLSASVGDRVTITCRASQSITSWLAWYQQKSGKAPKLLI | 911 |
| | Variable | YDASSLESGVPSRFSGSGSGTEFTLTISSLQPDDFATYYCQQYNSYPW | |
| | Region | TFGQGTKVEIK | |
| | LCDR1 | RASQSITSWLA | 912 |
| | LCDR2 | DASSLES | 913 |
| | LCDR3 | QQYNSYPWT | 914 |
| | LFRW1 | DIQMTQSPSTLSASVGDRVTITC | 915 |
| | LFRW2 | WYQQKSGKAPKLLIY | 916 |
| | LFRW3 | GVPSRFSGSGSGTEFTLTISSLQPDDFATYYC | 917 |
| | LFRW4 | FGQGTKVEIK | 918 |
| S144-469 | Heavy Chain | QVQLQESGPGLVKPSETLSLTCTVSGGSISSDYWSWIRQPPGKGLEWI | 919 |
| (ORF8) | | GYMYYSGSTNYNPSLKSRVTISVDTSKNQFSLKLSSVTAADTAVYYC | |
| | | ARWDRGSRPHYYYYGMDVWGQGTTVTVSSASTKGPSVFPLAPSSKS | |
| | | TSGGTAALGCLVKDYFPEPVTVSWNSGALTSGVHTFPAVLQSSG | |
| | Heavy Chain | QVQLQESGPGLVKPSETLSLTCTVSGGSISSDYWSWIRQPPGKGLEWI | 920 |
| | Variable | GYMYYSGSTNYNPSLKSRVTISVDTSKNQFSLKLSSVTAADTAVYYC | |
| | Region | ARWDRGSRPHYYYYGMDVWGQGTTVTVSS | |
| | HCDR1 | SDYWS | 921 |
| | HCDR2 | YMYYSGSTNYNPSLKS | 922 |
| | HCDR3 | WDRGSRPHYYYYGMDV | 923 |
| | HFRW1 | QVQLQESGPGLVKPSETLSLTCTVSGGSIS | 924 |
| | HFRW2 | WIRQPPGKGLEWIG | 925 |
| | HFRW3 | RVTISVDTSKNQFSLKLSSVTAADTAVYYCAR | 926 |
| | HFRW4 | WGQGTTVTVSS | 927 |
| | Light Chain | DIVMTQSPLSLPVTPGEPASISCRSSQSLLHSNGYNYLDWYLQKPGQS | 928 |
| | | PQLLIYLGSNRASGVPDRFSGSASGTDFTLKISRVEAEDVGVYYCMQA | |
| | | LQAFTFGPGTKVDIKRTVAAPSVFIFPPSDEQLKSGTASVVCLLNNFYP | |
| | | REAKVQWKVDNALQSGNSQESVTEQDSKDSTYSLSSTLTLSKADYE | |
| | Light Chain | DIVMTQSPLSLPVTPGEPASISCRSSQSLLHSNGYNYLDWYLQKPGQS | 929 |
| | Variable | PQLLIYLGSNRASGVPDRFSGSASGTDFTLKISRVEAEDVGVYYCMQA | |
| | Region | LQAFTFGPGTKVDIK | |
| | LCDR1 | RSSQSLLHSNGYNYLD | 930 |
| | LCDR2 | LGSNRAS | 931 |
| | LCDR3 | MQALQAFT | 932 |
| | LFRW1 | DIVMTQSPLSLPVTPGEPASISC | 933 |
| | LFRW2 | WYLQKPGQSPQLLIY | 934 |
| | LFRW3 | GVPDRFSGSASGTDFTLKISRVEAEDVGVYYC | 935 |
| | LFRW4 | FGPGTKVDIK | 936 |
| S144-509 | Heavy Chain | EVQLVQSGAEVKKPGESLKISCKGSAYTFTTYWIGWVRQMPGKGLE | 937 |
| (Spike/ | | WMGIIYPGDSDTRYSPSFQGQVTISADKSISTAYLQWSSLKASDTAMY | |
| RBD) | | YCARLLLVAGPFDYWGQGTLVTVSSASTKGPSVFPLAPSSKSTSGGT | |
| | | AALGCLVKDYFPEPVTVSWNSGALTSGVHTFPAVLQSSGLYSLSSVV | |
| | | TVPSSSLGTQTYICNVNHKPSNTKVD | |
| | Heavy Chain | EVQLVQSGAEVKKPGESLKISCKGSAYTFTTYWIGWVRQMPGKGLE | 938 |
| | Variable | WMGIIYPGDSDTRYSPSFQGQVTISADKSISTAYLQWSSLKASDTAMY | |
| | Region | YCARLLLVAGPFDYWGQGTLVTVSS | |
| | HCDR1 | TYWIG | 939 |
| | HCDR2 | IIYPGDSDTRYSPSFQG | 940 |
| | HCDR3 | LLLVAGPFDY | 941 |
| | HFRW1 | EVQLVQSGAEVKKPGESLKISCKGSAYTFT | 942 |
| | HFRW2 | WVRQMPGKGLEWMG | 943 |
| | HFRW3 | QVTISADKSISTAYLQWSSLKASDTAMYYCAR | 944 |
| | HFRW4 | WGQGTLVTVSS | 945 |
| | Light Chain | DIQMTQSPSTLSASVGDRVTITCRASQSISSWLAWYQQKPGKAPNLLI | 946 |
| | | YDASSLESGVPSRFSGSGSGTEFTLTISSLQPDDFATYYCQQYNSYPW | |
| | | TFGQGTKVEIKRTVAAPSVFIFPPSDEQLKSGTASVVCLLNNFYPREA | |
| | | KVQWKVDN | |
| | Light Chain | DIQMTQSPSTLSASVGDRVTITCRASQSISSWLAWYQQKPGKAPNLLI | 947 |
| | Variable | YDASSLESGVPSRFSGSGSGTEFTLTISSLQPDDFATYYCQQYNSYPW | |
| | Region | TFGQGTKVEIK | |
| | LCDR1 | RASQSISSWLA | 948 |
| | LCDR2 | DASSLES | 949 |
| | LCDR3 | QQYNSYPWT | 950 |
| | LFRW1 | DIQMTQSPSTLSASVGDRVTITC | 951 |
| | LFRW2 | WYQQKPGKAPNLLIY | 952 |
| | LFRW3 | GVPSRFSGSGSGTEFTLTISSLQPDDFATYYC | 953 |
| | LFRW4 | FGQGTKVEIK | 954 |
| S144-516 | Heavy Chain | QVQLLQSGAEVKKPGASVKVSCKASGYTFTGYYMHWVRQAPGQGL | 955 |
| (ORF8) | | EWMGRINPNSGGTNYAQKFQGRVTMTRDTSISTAYMELSRLTSDDTA | |
| | | VYYCATKTGIDRYYYYYMDVWGKGTTVTVSSASTKGPSVFPLAPSS | |
| | | KSTSGGTAALGCLVKDYFPEPVTVSWNSGALTSGVHTFPAVLQSSG | |
| | Heavy Chain | QVQLLQSGAEVKKPGASVKVSCKASGYTFTGYYMHWVRQAPGQGL | 956 |
| | Variable | EWMGRINPNSGGTNYAQKFQGRVTMTRDTSISTAYMELSRLTSDDTA | |
| | Region | VYYCATKTGIDRYYYYYMDVWGKGTTVTVSS | |
| | HCDR1 | GYYMH | 957 |
| | HCDR2 | RINPNSGGTNYAQKFQG | 958 |
| | HCDR3 | KTGIDRYYYYYMDV | 959 |
| | HFRW1 | QVQLLQSGAEVKKPGASVKVSCKASGYTFT | 960 |
| | HFRW2 | WVRQAPGQGLEWMG | 961 |
| | HFRW3 | RVTMTRDTSISTAYMELSRLTSDDTAVYYCAT | 962 |
| | HFRW4 | WGKGTTVTVSS | 963 |
| | Light Chain | QSVLTQPPSVSEAPGQRVTISCTGSSSNIGAGYDVHWYQQLPGTAPKL | 964 |
| | | LIYGNINRPSGVPDRFSGSKSGTSASLAITGLQAEDEADYYCQSYDNS | |
| | | LNGSVFGGGTKLTVLRQPKAAPSVTLFPPSSEELQANKATLVCLISDF | |
| | | YPGAVTVAWKADSSPVKAGVETTTPSKQSNNKYAASS | |
| | Light Chain | QSVLTQPPSVSEAPGQRVTISCTGSSSNIGAGYDVHWYQQLPGTAPKL | 965 |
| | Variable | LIYGNINRPSGVPDRFSGSKSGTSASLAITGLQAEDEADYYCQSYDNS | |
| | Region | LNGSVFGGGTKLTVL | |
| | LCDR1 | TGSSSNIGAGYDVH | 966 |
| | LCDR2 | GNINRPS | 967 |
| | LCDR3 | QSYDNSLNGSV | 968 |
| | LFRW1 | QSVLTQPPSVSEAPGQRVTISC | 969 |
| | LFRW2 | WYQQLPGTAPKLLIY | 970 |
| | LFRW3 | GVPDRFSGSKSGTSASLAITGLQAEDEADYYC | 971 |
| | LFRW4 | FGGGTKLTVL | 972 |
| S144-568 | Heavy Chain | QVQLQESGPGLVKPSETLSLTCSVSGGSISDYYWSWIRQPPGKGLEWI | 973 |
| (Spike/ | | GYIYNSGSTNYNPSLKSRVTISADPSKNQFSLKLSSVTAADTAVYYCA | |
| RBD) | | RPHGGDYAFDIWGQGTMVTVSSASPTSPKVFPLSLDSTPQDGNVVVA | |
| | | CLVQGFFPQEPLSVTWSESGQNVTARNF | |
| | Heavy Chain | QVQLQESGPGLVKPSETLSLTCSVSGGSISDYYWSWIRQPPGKGLEWI | 974 |
| | Variable | GYIYNSGSTNYNPSLKSRVTISADPSKNQFSLKLSSVTAADTAVYYCA | |
| | Region | RPHGGDYAFDIWGQGTMVTVSS | |
| | HCDR1 | DYYWS | 975 |
| | HCDR2 | YIYNSGSTNYNPSLKS | 976 |
| | HCDR3 | PHGGDYAFDI | 977 |
| | HFRW1 | QVQLQESGPGLVKPSETLSLTCSVSGGSIS | 978 |
| | HFRW2 | WIRQPPGKGLEWIG | 979 |
| | HFRW3 | RVTISADPSKNQFSLKLSSVTAADTAVYYCAR | 980 |
| | HFRW4 | WGQGTMVTVSS | 981 |
| | Light Chain | EIVLTQSPGTLSLSPGERATLSCRASQSVSSNFLAWYQQKPGQPPRLLI | 982 |
| | | YGASVRATGIPDRFSGSGSGTDFTLTITRLEPEDFAVYYCQQYGSLPRT | |
| | | FGQGTKVEIKRTVAAPSVFIFPPSDEQLKSGTASVVCLLNNFYPREAK | |
| | | VQWKVDNALQSGNSQESVTEQDSKDSTYSLSSTLTLSKADYE | |
| | Light Chain | EIVLTQSPGTLSLSPGERATLSCRASQSVSSNFLAWYQQKPGQPPRLLI | 983 |
| | Variable | YGASVRATGIPDRFSGSGSGTDFTLTITRLEPEDFAVYYCQQYGSLPRT | |
| | Region | FGQGTKVEIK | |
| | LCDR1 | RASQSVSSNFLA | 984 |
| | LCDR2 | GASVRAT | 985 |
| | LCDR3 | QQYGSLPRT | 986 |
| | LFRW1 | EIVLTQSPGTLSLSPGERATLSC | 987 |
| | LFRW2 | WYQQKPGQPPRLLIY | 988 |
| | LFRW3 | GIPDRFSGSGSGTDFTLTITRLEPEDFAVYYC | 989 |
| | LFRW4 | FGQGTKVEIK | 990 |
| S144-576 | Heavy Chain | QVQLVQSGAEVMKPGSSVKVSCKASGGTFSSYSITWVRQAPGQGLE | 991 |
| (Spike/ | | WMGRIIPILGIANYAQKFQGRVTITADKSTSTAYMELSSLRSEDTAVY | |
| RBD) | | YCARGYSGSPSNLDGMDVWGQGTTVTVSSASTKGPSVFPLAPSSKST | |
| | | SGGTAALGCLVKDYFPEPVTVSWNSGALTSGVHTFPAVLQSSG | |
| | Heavy Chain | QVQLVQSGAEVMKPGSSVKVSCKASGGTFSSYSITWVRQAPGQGLE | 992 |
| | Variable | WMGRIIPILGIANYAQKFQGRVTITADKSTSTAYMELSSLRSEDTAVY | |
| | Region | YCARGYSGSPSNLDGMDVWGQGTTVTVSS | |
| | HCDR1 | SYSIT | 993 |
| | HCDR2 | RIIPILGIANYAQKFQG | 994 |
| | HCDR3 | GYSGSPSNLDGMDV | 995 |
| | HFRW1 | QVQLVQSGAEVMKPGSSVKVSCKASGGTFS | 996 |
| | HFRW2 | WVRQAPGQGLEWMG | 997 |
| | HFRW3 | RVTITADKSTSTAYMELSSLRSEDTAVYYCAR | 998 |
| | HFRW4 | WGQGTTVTVSS | 999 |
| | Light Chain | IQMTQSPSTLSASVGDRVTITCRASQSISSWLAWYQQKPGKAPKLLIY | 1000 |
| | | DASSLQSGVPSRFSGSGSGTEFTLTISSLQPDDFATYYCQQYNSYSPITF | |
| | | GQGTRLEIKRTVAAPSVFIFPPSDEQLKSGTASVVCLLNNFYPREAKV | |
| | | QWKVDNALQSGNSQESVTEQDSKDSTYSLSSTLTLSKADYE | |
| | Light Chain | IQMTQSPSTLSASVGDRVTITCRASQSISSWLAWYQQKPGKAPKLLIY | 1001 |
| | Variable | DASSLQSGVPSRFSGSGSGTEFTLTISSLQPDDFATYYCQQYNSYSPITF | |
| | Region | GQGTRLEIK | |
| | LCDR1 | RASQSISSWLA | 1002 |
| | LCDR2 | DASSLQS | 1003 |
| | LCDR3 | QQYNSYSPIT | 1004 |
| | LFRW1 | IQMTQSPSTLSASVGDRVTITC | 1005 |
| | LFRW2 | WYQQKPGKAPKLLIY | 1006 |
| | LFRW3 | GVPSRFSGSGSGTEFTLTISSLQPDDFATYYC | 1007 |
| | LFRW4 | FGQGTRLEIK | 1008 |
| S144-588 | Heavy Chain | QLQLQESGPGLVKPSETLSLTCTVSGGSISSSSYYWGWIRQPPGKGLE | 1009 |
| (ORF8) | | WIGSIYYSGSTYYNPSLKSRFTISVDTSKNQFSLKLSSVTAADTAVYYC | |
| | | AAYQRKLGYCRGNSCFSCFDPWGQGTLVTVSSASTKGPSVFPLAPSS | |
| | | KSTSGGTAALGCLVKDYFPEPVTVSWNSGALTSGVHTFPAVLQSSG | |
| | Heavy Chain | QLQLQESGPGLVKPSETLSLTCTVSGGSISSSSYYWGWIRQPPGKGLE | 1010 |
| | Variable | WIGSIYYSGSTYYNPSLKSRFTISVDTSKNQFSLKLSSVTAADTAVYYC | |
| | Region | AAYQRKLGYCRGNSCFSCFDPWGQGTLVTVSS | |
| | HCDR1 | SSSYYWG | 1011 |
| | HCDR2 | SIYYSGSTYYNPSLKS | 1012 |
| | HCDR3 | YQRKLGYCRGNSCFSCFDP | 1013 |
| | HFRW1 | QLQLQESGPGLVKPSETLSLTCTVSGGSIS | 1014 |
| | HFRW2 | WIRQPPGKGLEWIG | 1015 |
| | HFRW3 | RFTISVDTSKNQFSLKLSSVTAADTAVYYCAA | 1016 |
| | HFRW4 | WGQGTLVTVSS | 1017 |
| | Light Chain | SYELTQPPSVSVSPGQTASITCSGDKLGDKYACWYQQKPGQSPVLVIY | 1018 |
| | | QDTKRPSGIPERFSGSNSGNTATLTISGTQAMDEADYYCQAWDSSTV | |
| | | LFGGGTKLTVLGQPKAAPSVTLFPPSSEELQANKATLVCLISDFYPGA | |
| | | VTVAWKADSSPVKAGVETTTPSKQSNNKYAASSYLSLTPEQWKSH | |
| | Light Chain | SYELTQPPSVSVSPGQTASITCSGDKLGDKYACWYQQKPGQSPVLVIY | 1019 |
| | Variable | QDTKRPSGIPERFSGSNSGNTATLTISGTQAMDEADYYCQAWDSSTV | |
| | Region | LFGGGTKLTVL | |
| | LCDR1 | SGDKLGDKYAC | 1020 |
| | LCDR2 | QDTKRPS | 1021 |
| | LCDR3 | QAWDSSTVL | 1022 |
| | LFRW1 | SYELTQPPSVSVSPGQTASITC | 1023 |
| | LFRW2 | WYQQKPGQSPVLVIY | 1024 |
| | LFRW3 | GIPERFSGSNSGNTATLTISGTQAMDEADYYC | 1025 |
| | LFRW4 | FGGGTKLTVL | 1026 |
| S144-628 | Heavy Chain | EVHLVQSGAEVKQPGESLKISCKGSGYNFATYWIAWVRQMPGKGLE | 1027 |
| (Spike/ | | WMGIIYPGDSDTRYSPSFQGQVIISADKSIGTAFLQWSSLKASDTAMY | |
| RBD) | | YCARRGYSSSNYRVDEYYYYGMDVWGQGTTVTVSSASPTSPKVFPL | |
| | | SLCSTQPDGNVVIACLVQGFFPQEPLSVTWSESGQGVTARNFP | |
| | Heavy Chain | EVHLVQSGAEVKQPGESLKISCKGSGYNFATYWIAWVRQMPGKGLE | 1028 |
| | Variable | WMGIIYPGDSDTRYSPSFQGQVIISADKSIGTAFLQWSSLKASDTAMY | |
| | Region | YCARRGYSSSNYRVDEYYYYGMDVWGQGTTVTVSS | |
| | HCDR1 | TYWIA | 1029 |
| | HCDR2 | IIYPGDSDTRYSPSFQG | 1030 |
| | HCDR3 | RGYSSSNYRVDEYYYYGMDV | 1031 |
| | HFRW1 | EVHLVQSGAEVKQPGESLKISCKGSGYNFA | 1032 |
| | HFRW2 | WVRQMPGKGLEWMG | 1033 |
| | HFRW3 | QVIISADKSIGTAFLQWSSLKASDTAMYYCAR | 1034 |
| | HFRW4 | WGQGTTVTVSS | 1035 |
| | Light Chain | QSVLTQPPSMSGAPGQRVTISCTGSSSNIGAGYDVHWYQQLPGAAPK | 1036 |
| | | LLIYGDTSRPSGVPDRFSGSKSDTSASLAITGLQAEDEADYYCQSFDRS | |
| | | LSGLVIFGGGTRLTVLGQPKAAPSVTLFPPSSEELQANKATLVCLISDF | |
| | | YPGAVTVAWKADSSPVKAGVETTTPSKQSNNKYAASS*DRKS | |
| | Light Chain | QSVLTQPPSMSGAPGQRVTISCTGSSSNIGAGYDVHWYQQLPGAAPK | 1037 |
| | Variable | LLIYGDTSRPSGVPDRFSGSKSDTSASLAITGLQAEDEADYYCQSFDRS | |
| | Region | LSGLVIFGGGTRLTVL | |
| | LCDR1 | TGSSSNIGAGYDVH | 1038 |
| | LCDR2 | GDTSRPS | 1039 |
| | LCDR3 | QSFDRSLSGLVI | 1040 |
| | LFRW1 | QSVLTQPPSMSGAPGQRVTISC | 1041 |
| | LFRW2 | WYQQLPGAAPKLLIY | 1042 |
| | LFRW3 | GVPDRFSGSKSDTSASLAITGLQAEDEADYYC | 1043 |
| | LFRW4 | FGGGTRLTVL | 1044 |
| S144-740 | Heavy Chain | QVQLVQSGAEVKKPGASVKVSCKASGYTFTGYYMHWVRQAPGQGL | 1045 |
| (ORF8) | | EWMGRINPNSGDTNYAQKFQGRVTMTRDTSISTAYMELSRLRSDDT | |
| | | AVYYCARLGKGMAAARTVFDSWGQGTLVTVSSASTKGPSVFPLAPS | |
| | | SKSTSGGTAALGCLVKDYFPEPVTVSWNSGALTSGVHTFPAVLQSSG | |
| | Heavy Chain | QVQLVQSGAEVKKPGASVKVSCKASGYTFTGYYMHWVRQAPGQGL | 1046 |
| | Variable | EWMGRINPNSGDTNYAQKFQGRVTMTRDTSISTAYMELSRLRSDDT | |
| | Region | AVYYCARLGKGMAAARTVFDSWGQGTLVTVSS | |
| | HCDR1 | GYYMH | 1047 |
| | HCDR2 | RINPNSGDTNYAQKFQG | 1048 |
| | HCDR3 | LGKGMAAARTVFDS | 1049 |
| | HFRW1 | QVQLVQSGAEVKKPGASVKVSCKASGYTFT | 1050 |
| | HFRW2 | WVRQAPGQGLEWMG | 1051 |
| | HFRW3 | RVTMTRDTSISTAYMELSRLRSDDTAVYYCAR | 1052 |
| | HFRW4 | WGQGTLVTVSS | 1053 |
| | Light Chain | EVVLTQSPGTLSLSPGERATLSCRASQSVSSSYLAWYQQKPGQAPRLV | 1054 |
| | | IYGASSRATGIPDRFSGSGSGTDFTLTISRLEPEDFAVYYCQQFGSSPTF | |
| | | GRGTRLEIKRTVAAPSVFIFPPSDEQLKSGTASVVCLLNNFYPREAKV | |
| | | QWKVDNALQSGNSQESVTEQDSKDSTYSLSSTLTLSKADYE | |
| | Light Chain | EVVLTQSPGTLSLSPGERATLSCRASQSVSSSYLAWYQQKPGQAPRLV | 1055 |
| | Variable | IYGASSRATGIPDRFSGSGSGTDFTLTISRLEPEDFAVYYCQQFGSSPTF | |
| | Region | GRGTRLEIK | |
| | LCDR1 | RASQSVSSSYLA | 1056 |
| | LCDR2 | GASSRAT | 1057 |
| | LCDR3 | QQFGSSPT | 1058 |
| | LFRW1 | EVVLTQSPGTLSLSPGERATLSC | 1059 |
| | LFRW2 | WYQQKPGQAPRLVIY | 1060 |
| | LFRW3 | GIPDRFSGSGSGTDFTLTISRLEPEDFAVYYC | 1061 |
| | LFRW4 | FGRGTRLEIK | 1062 |
| S144-741 | Heavy Chain | QVHLVQSGAEVKKPGASVKVSCKASGYTFTGYYMNWVRQAPGQGL | 1063 |
| (ORF8) | | EWMGRINPNSGGTNYAQKFQGRVTMTRDTSISTAYMELSRLRSDDA | |
| | | AVYYCARAERYSSSWYNLYYWGQGTLVTVSSASTKGPSVFPLAPSSK | |
| | | STSGGTAALGCLVKDYFPEPVTVSWNSGALTSGVHTFPAVLQSSG | |
| | Heavy Chain | QVHLVQSGAEVKKPGASVKVSCKASGYTFTGYYMNWVRQAPGQGL | 1064 |
| | Variable | EWMGRINPNSGGTNYAQKFQGRVTMTRDTSISTAYMELSRLRSDDA | |
| | Region | AVYYCARAERYSSSWYNLYYWGQGTLVTVSS | |
| | HCDR1 | GYYMN | 1065 |
| | HCDR2 | RINPNSGGTNYAQKFQG | 1066 |
| | HCDR3 | AERYSSSWYNLYY | 1067 |
| | HFRW1 | QVHLVQSGAEVKKPGASVKVSCKASGYTFT | 1068 |
| | HFRW2 | WVRQAPGQGLEWMG | 1069 |
| | HFRW3 | RVTMTRDTSISTAYMELSRLRSDDAAVYYCAR | 1070 |
| | HFRW4 | WGQGTLVTVSS | 1071 |
| | Light Chain | QSVLTQPPSASGTPGQRVTISCSGSSSNIGSNTVNWYQQLPGTAPKLLI | 1072 |
| | | YSNNQRPSGVPDRFSGSKSGTSASLAISGLQSEDEADYYCAAWDDSL | |
| | | NGVVFGGGTKLTVLGQPKAAPSVTLFPPSSEELQANKATLVCLISDFY | |
| | | PGAVTVAWKADSSPVKAGVETTTPSKQSNNKYAASSYLSLTPEQWK | |
| | | SH | |
| | Light Chain | QSVLTQPPSASGTPGQRVTISCSGSSSNIGSNTVNWYQQLPGTAPKLLI | 1073 |
| | Variable | YSNNQRPSGVPDRFSGSKSGTSASLAISGLQSEDEADYYCAAWDDSL | |
| | Region | NGVVFGGGTKLTVL | |
| | LCDR1 | SGSSSNIGSNTVN | 1074 |
| | LCDR2 | SNNQRPS | 1075 |
| | LCDR3 | AAWDDSLNGVV | 1076 |
| | LFRW1 | QSVLTQPPSASGTPGQRVTISC | 1077 |
| | LFRW2 | WYQQLPGTAPKLLIY | 1078 |
| | LFRW3 | GVPDRFSGSKSGTSASLAISGLQSEDEADYYC | 1079 |
| | LFRW4 | FGGGTKLTVL | 1080 |
| S144-803 | Heavy Chain | EVQLVQSGAEVKKPGESLKISCKGSRYSFTRYWIAWVRQMPGKGLE | 1081 |
| (Spike/ | | WMGIIYPGDSDTRYSPSFQGPVTISADKSISTAYLQWSSLKASDTAIYY | |
| RBD) | | CARLPNSNYVDYWGQGTLVTVSSASTKGPSVFPLAPSSKSTSGGTAA | |
| | | LGCLVKDYFPEPVTVSWNSGALTSGVHTFPAVLQSSGLYSLSSVVTVP | |
| | | SSSLGTQTYICNVNHKPSNTKVD | |
| | Heavy Chain | EVQLVQSGAEVKKPGESLKISCKGSRYSFTRYWIAWVRQMPGKGLE | 1082 |
| | Variable | WMGIIYPGDSDTRYSPSFQGPVTISADKSISTAYLQWSSLKASDTAIYY | |
| | Region | CARLPNSNYVDYWGQGTLVTVSS | |
| | HCDR1 | RYWIA | 1083 |
| | HCDR2 | IIYPGDSDTRYSPSFQG | 1084 |
| | HCDR3 | LPNSNYVDY | 1085 |
| | HFRW1 | EVQLVQSGAEVKKPGESLKISCKGSRYSFT | 1086 |
| | HFRW2 | WVRQMPGKGLEWMG | 1087 |
| | HFRW3 | PVTISADKSISTAYLQWSSLKASDTAIYYCAR | 1088 |
| | HFRW4 | WGQGTLVTVSS | 1089 |
| | Light Chain | DIQMTQSPSTLSASVGDRVTITCRASQSISSWLAWYQQKPGKAPKLLI | 1090 |
| | | YDASSLESGVPSRFSGSGSGTEFTLTISSLQPDDFATYYCQQYNIYPYT | |
| | | FGQGTKLDIKRTVAAPSVFIFPPSDEQLKSGTASVVCLLNNFYPREAK | |
| | | VQWKVDNALQSGNSQESVTEQDSKDSTYSLSSTLTLSKADYE | |
| | Light Chain | DIQMTQSPSTLSASVGDRVTITCRASQSISSWLAWYQQKPGKAPKLLI | 1091 |
| | Variable | YDASSLESGVPSRFSGSGSGTEFTLTISSLQPDDFATYYCQQYNIYPYT | |
| | Region | FGQGTKLDIK | |
| | LCDR1 | RASQSISSWLA | 1092 |
| | LCDR2 | DASSLES | 1093 |
| | LCDR3 | QQYNIYPYT | 1094 |
| | LFRW1 | DIQMTQSPSTLSASVGDRVTITC | 1095 |
| | LFRW2 | WYQQKPGKAPKLLIY | 1096 |
| | LFRW3 | GVPSRFSGSGSGTEFTLTISSLQPDDFATYYC | 1097 |
| | LFRW4 | FGQGTKLDIK | 1098 |
| S144-843 | Heavy Chain | QVQLVESGGGVVQPGGSVRLSCAASGFDFTNNGMYWVRQAPGKGL | 1099 |
| (ORF8) | | EWVAFIRYDGNKQDYADSVKGRFTISRDNSKNTLYLQMSSLRPEDTA | |
| | | VYYCAKGVYTENYGWGQGTLVTVSSGTTVTVSSASTKGPSVFPLAPC | |
| | | SRSTSESTAALGCLVKDYFPEPVTVSWNSGALTSGVHTFPAVLQSSGL | |
| | | YSLSSVVTVPSSNFGTQTYTCNVDHKPSNTKVD | |
| | Heavy Chain | QVQLVESGGGVVQPGGSVRLSCAASGFDFTNNGMYWVRQAPGKGL | 1100 |
| | Variable | EWVAFIRYDGNKQDYADSVKGRFTISRDNSKNTLYLQMSSLRPEDTA | |
| | Region | VYYCAKGVYTENYGWGQGTLVTVSS | |
| | HCDR1 | NNGMY | 1101 |
| | HCDR2 | FIRYDGNKQDYADSVKG | 1102 |
| | HCDR3 | GVYTENYG | 1103 |
| | HFRW1 | QVQLVESGGGVVQPGGSVRLSCAASGFDFT | 1104 |
| | HFRW2 | WVRQAPGKGLEWVA | 1105 |
| | HFRW3 | RFTISRDNSKNTLYLQMSSLRPEDTAVYYCAK | 1106 |
| | HFRW4 | WGQGTLVTVSS | 1107 |
| | Light Chain | EIVLTQSPGTLSLSPGERATLSCRASQTVTSRYLAWYQQKPGQAPRLL | 1108 |
| | | IYGASTRATGIPDRFSGSGSGTDFTLTISRLEPEDFAVYYCQQYGNSPP | |
| | | YTFGQGTKLEIKRTVAAPSVFIFPPSDEQLKSGTASVVCLLNNFYPREA | |
| | | KVQWKVDNALQSGNSQESVTEQDSKDSTYSLSSTLTLSKADYE | |
| | Light Chain | EIVLTQSPGTLSLSPGERATLSCRASQTVTSRYLAWYQQKPGQAPRLL | 1109 |
| | Variable | IYGASTRATGIPDRFSGSGSGTDFTLTISRLEPEDFAVYYCQQYGNSPP | |
| | Region | YTFGQGTKLEIK | |
| | LCDR1 | RASQTVTSRYLA | 1110 |
| | LCDR2 | GASTRAT | 1111 |
| | LCDR3 | QQYGNSPPYT | 1112 |
| | LFRW1 | EIVLTQSPGTLSLSPGERATLSC | 1113 |
| | LFRW2 | WYQQKPGQAPRLLIY | 1114 |
| | LFRW3 | GIPDRFSGSGSGTDFTLTISRLEPEDFAVYYC | 1115 |
| | LFRW4 | FGQGTKLEIK | 1116 |
| S144-877 | Heavy Chain | QVQLVESGGGVVQPGRSLRLSCAASGFTFSTYGMHWVRQAPGKGLE | 1117 |
| (Spike/ | | WVAVISYDGSNKYYADSVKGRFTISRDNSKNTLYLQMNSLRAEDTA | |
| RBD) | | VYYCAKQQGTYCSGGNCYSGYFDYWGQGTLVTVSSASTKGPSVFPL | |
| | | APSSKSTSGGTAALGCLVKDYFPEPVTVSWNSGALTSGVHTFPAVLQ | |
| | | SSGLYSLSSVVTVPSSSLGTQTYIC | |
| | Heavy Chain | QVQLVESGGGVVQPGRSLRLSCAASGFTFSTYGMHWVRQAPGKGLE | 1118 |
| | Variable | WVAVISYDGSNKYYADSVKGRFTISRDNSKNTLYLQMNSLRAEDTA | |
| | Region | VYYCAKQQGTYCSGGNCYSGYFDYWGQGTLVTVSS | |
| | HCDR1 | TYGMH | 1119 |
| | HCDR2 | VISYDGSNKYYADSVKG | 1120 |
| | HCDR3 | QQGTYCSGGNCYSGYFDY | 1121 |
| | HFRW1 | QVQLVESGGGVVQPGRSLRLSCAASGFTFS | 1122 |
| | HFRW2 | WVRQAPGKGLEWVA | 1123 |
| | HFRW3 | RFTISRDNSKNTLYLQMNSLRAEDTAVYYCAK | 1124 |
| | HFRW4 | WGQGTLVTVSS | 1125 |
| | Light Chain | DIQMTQSPSSLSASVGDRVTITCQASQDISNYLNWYQQKPGKAPKLLI | 1126 |
| | | YDASNLETGVPSRFSGSGSGTDFSFSISSLQPEDIATYYCQQYDNVPLT | |
| | | FGGGTKVEIKRTVAAPSVFIFPPSDEQLKSGTASVVCLLNNFYPREAK | |
| | | VQWKVDNALQSGNSQESVTEQDSKDSTYSLSSTLTLSKADYE | |
| | Light Chain | DIQMTQSPSSLSASVGDRVTITCQASQDISNYLNWYQQKPGKAPKLLI | 1127 |
| | Variable | YDASNLETGVPSRFSGSGSGTDFSFSISSLQPEDIATYYCQQYDNVPLT | |
| | Region | FGGGTKVEIK | |
| | LCDR1 | QASQDISNYLN | 1128 |
| | LCDR2 | DASNLET | 1129 |
| | LCDR3 | QQYDNVPLT | 1130 |
| | LFRW1 | DIQMTQSPSSLSASVGDRVTITC | 1131 |
| | LFRW2 | WYQQKPGKAPKLLIY | 1132 |
| | LFRW3 | GVPSRFSGSGSGTDFSFSISSLQPEDIATYYC | 1133 |
| | LFRW4 | FGGGTKVEIK | 1134 |
| S144-952 | Heavy Chain | QVQLVQSGAEVKKPGASVKVSCTASGYTVTSYGISWVRQAPGQGLE | 1135 |
| (NP) | | WMGWISTYNGNTNYAQKLQGRVTMTTDTSTSTAYMELRSLRSDDT | |
| | | AVYYCAREYSYGYRLAYFDYWGQGTLVTVSSGSASAPTLFPLVSCEN | |
| | | SPSDTSSVAVGCLAQDFLPDSITFSWKYKNNSDISSTRGFPSVLRGGK | |
| | | YAATSQVLLPSKDVM | |
| | Heavy Chain | QVQLVQSGAEVKKPGASVKVSCTASGYTVTSYGISWVRQAPGQGLE | 1136 |
| | Variable | WMGWISTYNGNTNYAQKLQGRVTMTTDTSTSTAYMELRSLRSDDT | |
| | Region | AVYYCAREYSYGYRLAYFDYWGQGTLVTVSS | |
| | HCDR1 | SYGIS | 1137 |
| | HCDR2 | WISTYNGNTNYAQKLQG | 1138 |
| | HCDR3 | EYSYGYRLAYFDY | 1139 |
| | HFRW1 | QVQLVQSGAEVKKPGASVKVSCTASGYTVT | 1140 |
| | HFRW2 | WVRQAPGQGLEWMG | 1141 |
| | HFRW3 | RVTMTTDTSTSTAYMELRSLRSDDTAVYYCAR | 1142 |
| | HFRW4 | WGQGTLVTVSS | 1143 |
| | Light Chain | DIVMTQSPDSLAVSLGERATINCKSSQSVLNSSNNKNYLAWYQQKPG | 1144 |
| | | QPPKLLIYWASTRESGVPDRFSGSGSGTDFTLTISSLQAEDVAVYYCQ | |
| | | QYYSTPQTFGQGTKVEIKRTVAAPSVFIFPPSDEQLKSGTASVVCLLN | |
| | | NFYPREAKVQWKVDNALQSGNSQESVTEQDSKDSTYSLSSTLTLSKA | |
| | | DYE | |
| | Light Chain | DIVMTQSPDSLAVSLGERATINCKSSQSVLNSSNNKNYLAWYQQKPG | 1145 |
| | Variable | QPPKLLIYWASTRESGVPDRFSGSGSGTDFTLTISSLQAEDVAVYYCQ | |
| | Region | QYYSTPQTFGQGTKVEIK | |
| | LCDR1 | KSSQSVLNSSNNKNYLA | 1146 |
| | LCDR2 | WASTRES | 1147 |
| | LCDR3 | QQYYSTPQT | 1148 |
| | LFRW1 | DIVMTQSPDSLAVSLGERATINC | 1149 |
| | LFRW2 | WYQQKPGQPPKLLIY | 1150 |
| | LFRW3 | GVPDRFSGSGSGTDFTLTISSLQAEDVAVYYC | 1151 |
| | LFRW4 | FGQGTKVEIK | 1152 |
| S144-971 | Heavy Chain | EVQLVESGGGLVQPGGSLRISCSASGFTFSRYAMHWVRQAPGKGLEY | 1153 |
| (ORF8) | | VSAIRSNGGSTYYADSVRGRFTISRDNSRNTLYLQMSSLRAEDTAVY | |
| | | YCVIINNLAAAGTRFDYWGQGTLVTVSSASTKGPSVFPLAPSSKSTSG | |
| | | GTAALGCLVKDYFPEPVTVSWNSGALTSGVHTFPAVLQSSG | |
| | Heavy Chain | EVQLVESGGGLVQPGGSLRISCSASGFTFSRYAMHWVRQAPGKGLEY | 1154 |
| | Variable | VSAIRSNGGSTYYADSVRGRFTISRDNSRNTLYLQMSSLRAEDTAVY | |
| | Region | YCVIINNLAAAGTRFDYWGQGTLVTVSS | |
| | HCDR1 | RYAMH | 1155 |
| | HCDR2 | AIRSNGGSTYYADSVRG | 1156 |
| | HCDR3 | INNLAAAGTRFDY | 1157 |
| | HFRW1 | EVQLVESGGGLVQPGGSLRISCSASGFTFS | 1158 |
| | HFRW2 | WVRQAPGKGLEYVS | 1159 |
| | HFRW3 | RFTISRDNSRNTLYLQMSSLRAEDTAVYYCVI | 1160 |
| | HFRW4 | WGQGTLVTVSS | 1161 |
| | Light Chain | DIVMTQSPDSLAVSLGERATINCKSSQSVLYSSNNKNFLTWYQQKPG | 1162 |
| | | QPPKLLIYWASTRESGVPDRFSGSGSGTDFTLTISSLQAEDVAVYYCQ | |
| | | QYYTTPWTFGQGTKVEIKRTVAAPSVFIFPPSDEQLKSGTASVVCLLN | |
| | | NFYPREAKVQWKVDNALQSGNSQESVTEQDSKDSTYSLSSTLTLSKA | |
| | | DYE | |
| | Light Chain | DIVMTQSPDSLAVSLGERATINCKSSQSVLYSSNNKNFLTWYQQKPG | 1163 |
| | Variable | QPPKLLIYWASTRESGVPDRFSGSGSGTDFTLTISSLQAEDVAVYYCQ | |
| | Region | QYYTTPWTFGQGTKVEIK | |
| | LCDR1 | KSSQSVLYSSNNKNFLT | 1164 |
| | LCDR2 | WASTRES | 1165 |
| | LCDR3 | QQYYTTPWT | 1166 |
| | LFRW1 | DIVMTQSPDSLAVSLGERATINC | 1167 |
| | LFRW2 | WYQQKPGQPPKLLIY | 1168 |
| | LFRW3 | GVPDRFSGSGSGTDFTLTISSLQAEDVAVYYC | 1169 |
| | LFRW4 | FGQGTKVEIK | 1170 |
| S144-1036 | Heavy Chain | QVQLQQWGAGLLKPSETLSLTCAVYGGSFSGYFWSWIRQPPGKGLE | 1171 |
| (NP) | | WIGEINHSGSTNYNPSLKSRVTISVDTSKNQFSLKLSSVTAADTAVYY | |
| | | CARAPYYDFLREGNWFDPWGQGTLVTVSSASTKGPSVFPLAPSSKST | |
| | | SGGTAALGCLVKDYFPEPVTVSWNSGALTSGVHTFPAVLQSSGLYSL | |
| | | SSVVTVPSSSLGTQTYICNVNHKPS | |
| | Heavy Chain | QVQLQQWGAGLLKPSETLSLTCAVYGGSFSGYFWSWIRQPPGKGLE | 1172 |
| | Variable | WIGEINHSGSTNYNPSLKSRVTISVDTSKNQFSLKLSSVTAADTAVYY | |
| | Region | CARAPYYDFLREGNWFDPWGQGTLVTVSS | |
| | HCDR1 | GYFWS | 1173 |
| | HCDR2 | EINHSGSTNYNPSLKS | 1174 |
| | HCDR3 | APYYDFLREGNWFDP | 1175 |
| | HFRW1 | QVQLQQWGAGLLKPSETLSLTCAVYGGSFS | 1176 |
| | HFRW2 | WIRQPPGKGLEWIG | 1177 |
| | HFRW3 | RVTISVDTSKNQFSLKLSSVTAADTAVYYCAR | 1178 |
| | HFRW4 | WGQGTLVTVSS | 1179 |
| | Light Chain | DIVMTQSPDSLAVSLGERATINCNSSQSVLYSSINKNYLAWYQQKPA | 1180 |
| | | QPPKVLIYWASTRESGVPDRFSGSGSGTDFTLTISSLQAEDVAVYYCQ | |
| | | QYYRTPWTFGQGTKVEIKRTVAAPSVFIFPPSDEQLKSGTASVVCLLN | |
| | | NFYPREAKVQWKVDNALQSGNSQESVTEQDSKDSTYSLSSTLTLSKA | |
| | | DYE | |
| | Light Chain | DIVMTQSPDSLAVSLGERATINCNSSQSVLYSSINKNYLAWYQQKPA | 1181 |
| | Variable | QPPKVLIYWASTRESGVPDRFSGSGSGTDFTLTISSLQAEDVAVYYCQ | |
| | Region | QYYRTPWTFGQGTKVEIK | |
| | LCDR1 | NSSQSVLYSSINKNYLA | 1182 |
| | LCDR2 | WASTRES | 1183 |
| | LCDR3 | QQYYRTPWT | 1184 |
| | LFRW1 | DIVMTQSPDSLAVSLGERATINC | 1185 |
| | LFRW2 | WYQQKPAQPPKVLIY | 1186 |
| | LFRW3 | GVPDRFSGSGSGTDFTLTISSLQAEDVAVYYC | 1187 |
| | LFRW4 | FGQGTKVEIK | 1188 |
| S144-1079 | Heavy Chain | QVQLVQSGAEVKKPGSSVKVSCKASGDTFGSYSITWVRQAPGQGLE | 1189 |
| (Spike/ | | WMGRIIPVLGIANYAQKFQGRVTITADKSTSTAYMELSSLRSEDTAVY | |
| RBD) | | YCAGGGCSGGNCYSWYNWFDPWGQGSLVTVSSASTKGPSVFPLAPS | |
| | | SKSTSGGTAALGCLVKDYFPEPVTVSWNSGALTSGVHTFPAVLQSSG | |
| | Heavy Chain | QVQLVQSGAEVKKPGSSVKVSCKASGDTFGSYSITWVRQAPGQGLE | 1190 |
| | Variable | WMGRIIPVLGIANYAQKFQGRVTITADKSTSTAYMELSSLRSEDTAVY | |
| | Region | YCAGGGCSGGNCYSWYNWFDPWGQGSLVTVSS | |
| | HCDR1 | SYSIT | 1191 |
| | HCDR2 | RIIPVLGIANYAQKFQG | 1192 |
| | HCDR3 | GGCSGGNCYSWYNWFDP | 1193 |
| | HFRW1 | QVQLVQSGAEVKKPGSSVKVSCKASGDTFG | 1194 |
| | HFRW2 | WVRQAPGQGLEWMG | 1195 |
| | HFRW3 | RVTITADKSTSTAYMELSSLRSEDTAVYYCAG | 1196 |
| | HFRW4 | WGQGSLVTVSS | 1197 |
| | Light Chain | EIVLTQSPGTLSLSPGERATLSCRASQSVSSNYLAWYQQKPGQAPRLLI | 1198 |
| | | YGASSRATGIPERFSGSGSGTDFTLTISRLEPEDFAVYYCQQYGRSPYT | |
| | | FGQGTKLEIKRTVAAPSVFIFPPSDEQLKSGTASVVCLLNNFYPREAK | |
| | | VQWKVDNALQSGNSQESVTEQDSKDSTYSLSSTLTLSKADYE | |
| | Light Chain | EIVLTQSPGTLSLSPGERATLSCRASQSVSSNYLAWYQQKPGQAPRLLI | 1199 |
| | Variable | YGASSRATGIPERFSGSGSGTDFTLTISRLEPEDFAVYYCQQYGRSPYT | |
| | Region | FGQGTKLEIK | |
| | LCDR1 | RASQSVSSNYLA | 1200 |
| | LCDR2 | GASSRAT | 1201 |
| | LCDR3 | QQYGRSPYT | 1202 |
| | LFRW1 | EIVLTQSPGTLSLSPGERATLSC | 1203 |
| | LFRW2 | WYQQKPGQAPRLLIY | 1204 |
| | LFRW3 | GIPERFSGSGSGTDFTLTISRLEPEDFAVYYC | 1205 |
| | LFRW4 | FGQGTKLEIK | 1206 |
| S144-1299 | Heavy Chain | QVQLQESGPGLVKPSETLSLTCTVSGGSISSYYWSWIRQPPGKGLEWI | 1207 |
| (ORF8) | | GYINYRGITNYNPSLKSRVTISVDMSKNQFSLKLSSVTAADTAVYSCA | |
| | | RLAVASRGTVDYWGQGTLVTVSSASTKGPSVFPLAPSSKSTSGGTAA | |
| | | LGCLVKDYFPEPVTVSWNSGALTSGVHTFPAVLQSSGLYSLSSVVTVP | |
| | | SSNFGTQTYTCNVDHKPSNTKVD | |
| | Heavy Chain | QVQLQESGPGLVKPSETLSLTCTVSGGSISSYYWSWIRQPPGKGLEWI | 1208 |
| | Variable | GYINYRGITNYNPSLKSRVTISVDMSKNQFSLKLSSVTAADTAVYSCA | |
| | Region | RLAVASRGTVDYWGQGTLVTVSS | |
| | HCDR1 | SYYWS | 1209 |
| | HCDR2 | YINYRGITNYNPSLKS | 1210 |
| | HCDR3 | LAVASRGTVDY | 1211 |
| | HFRW1 | QVQLQESGPGLVKPSETLSLTCTVSGGSIS | 1212 |
| | HFRW2 | WIRQPPGKGLEWIG | 1213 |
| | HFRW3 | RVTISVDMSKNQFSLKLSSVTAADTAVYSCAR | 1214 |
| | HFRW4 | WGQGTLVTVSS | 1215 |
| | Light Chain | QSVLTQPPSASGTPGQRVTISCSGSSSNIGSNYVYWYQQLPGTAPKLLI | 1216 |
| | | YRNNQRPSGVPDRFSGSKSGTSASLAISGLRSEDEADYYCAAWDDSL | |
| | | SVNVVFGGGTKLTVLGQPKAAPSVTLFPPSSEELQANKATLVCLISDF | |
| | | YPGAVTVAWKADSSPVKAGVETTKPSKQSNNKYAASSYLSLTPEQW | |
| | | KSH | |
| | Light Chain | QSVLTQPPSASGTPGQRVTISCSGSSSNIGSNYVYWYQQLPGTAPKLLI | 1217 |
| | Variable | YRNNQRPSGVPDRFSGSKSGTSASLAISGLRSEDEADYYCAAWDDSL | |
| | Region | SVNVVFGGGTKLTVL | |
| | LCDR1 | SGSSSNIGSNYVY | 1218 |
| | LCDR2 | RNNQRPS | 1219 |
| | LCDR3 | AAWDDSLSVNVV | 1220 |
| | LFRW1 | QSVLTQPPSASGTPGQRVTISC | 1221 |
| | LFRW2 | WYQQLPGTAPKLLIY | 1222 |
| | LFRW3 | GVPDRFSGSKSGTSASLAISGLRSEDEADYYC | 1223 |
| | LFRW4 | FGGGTKLTVL | 1224 |
| S144-1339 | Heavy Chain | QVQLVQSGTEVKKPGASVKVSCKASGYTFTDYYMHWVRQAPGQGL | 1225 |
| (Spike/ | | EWMGRINPTSGGTNYPQKFQGSVTMTRDTSLSTVYMELSGLRSDDTA | |
| RBD) | | VYYCARERVTLIQGKNHYYMDVWGTGTTVTVSSASTKGPSVFPLAPS | |
| | | SKSTSGGTAALGCLVKDYFPEPVTVSWNSGALTSGVHTFPAVLQSSG | |
| | Heavy Chain | QVQLVQSGTEVKKPGASVKVSCKASGYTFTDYYMHWVRQAPGQGL | 1226 |
| | Variable | EWMGRINPTSGGTNYPQKFQGSVTMTRDTSLSTVYMELSGLRSDDTA | |
| | Region | VYYCARERVTLIQGKNHYYMDVWGTGTTVTVSS | |
| | HCDR1 | DYYMH | 1227 |
| | HCDR2 | RINPTSGGTNYPQKFQG | 1228 |
| | HCDR3 | ERVTLIQGKNHYYMDV | 1229 |
| | HFRW1 | QVQLVQSGTEVKKPGASVKVSCKASGYTFT | 1230 |
| | HFRW2 | WVRQAPGQGLEWMG | 1231 |
| | HFRW3 | SVTMTRDTSLSTVYMELSGLRSDDTAVYYCAR | 1232 |
| | HFRW4 | WGTGTTVTVSS | 1233 |
| | Light Chain | QSALTQPASVSGSPGQSITISCTGTNSDVGGYNYVSWYQQHPGKAPR | 1234 |
| | | LMIYDVSNRPSGVSNRFSGSKSGNTASLTISGLQAEDEADYYCSSYTS | |
| | | SSTLVVFGGGTKLTVLGQPKAAPSVTLFPPSSEELQANKATLVCLISDF | |
| | | YPGAVTVAWKADSSPVKAGVETTTPSKQSNNKYAASSYLSLTPEQW | |
| | | KSH | |
| | Light Chain | QSALTQPASVSGSPGQSITISCTGTNSDVGGYNYVSWYQQHPGKAPR | 1235 |
| | Variable | LMIYDVSNRPSGVSNRFSGSKSGNTASLTISGLQAEDEADYYCSSYTS | |
| | Region | SSTLVVFGGGTKLTVL | |
| | LCDR1 | TGTNSDVGGYNYVS | 1236 |
| | LCDR2 | DVSNRPS | 1237 |
| | LCDR3 | SSYTSSSTLVV | 1238 |
| | LFRW1 | QSALTQPASVSGSPGQSITISC | 1239 |
| | LFRW2 | WYQQHPGKAPRLMIY | 1240 |
| | LFRW3 | GVSNRFSGSKSGNTASLTISGLQAEDEADYYC | 1241 |
| | LFRW4 | FGGGTKLTVL | 1242 |
| S144-1406 | Heavy Chain | QVQLVQSGAEVKKPGASVKVSCKASGYTFTTYAMHWVRQAPGQRL | 1243 |
| (Spike/ | | EWMGWINAGNGNTKYSQNFQGRVTITRDTSASTAYMELSSLRSEDT | |
| RBD) | | AVYYCASLVGGDSSSWYDYMDVWGKGTTVTVSSASTKGPSVFPLAP | |
| | | CSRSTSESTAALGCLVKDYFPEPVTVSWNSGALTSGVHTFPAVLQSSG | |
| | | LYSLSSVVTVPSSNF | |
| | Heavy Chain | QVQLVQSGAEVKKPGASVKVSCKASGYTFTTYAMHWVRQAPGQRL | 1244 |
| | Variable | EWMGWINAGNGNTKYSQNFQGRVTITRDTSASTAYMELSSLRSEDT | |
| | Region | AVYYCASLVGGDSSSWYDYMDVWGKGTTVTVSS | |
| | HCDR1 | TYAMH | 1245 |
| | HCDR2 | WINAGNGNTKYSQNFQG | 1246 |
| | HCDR3 | LVGGDSSSWYDYMDV | 1247 |
| | HFRW1 | QVQLVQSGAEVKKPGASVKVSCKASGYTFT | 1248 |
| | HFRW2 | WVRQAPGQRLEWMG | 1249 |
| | HFRW3 | RVTITRDTSASTAYMELSSLRSEDTAVYYCAS | 1250 |
| | HFRW4 | WGKGTTVTVSS | 1251 |
| | Light Chain | DIQMTQSPSTLSASVGDRVTITCRASQSISSWLAWYQQKPGKAPKLLI | 1252 |
| | | YDASSLESGVPSRFSGSGSGTEFTLTISSLQPDDFATYYCQQYNSYPW | |
| | | TFGQGTKVEIKRTVAAPSVFIFPPSDEQLKSGTASVVCLLNNFYPREA | |
| | | KVQWKVDNALQSGNSQESVTEQDSKDSTYSLSSTLTLSKADYE | |
| | Light Chain | DIQMTQSPSTLSASVGDRVTITCRASQSISSWLAWYQQKPGKAPKLLI | 1253 |
| | Variable | YDASSLESGVPSRFSGSGSGTEFTLTISSLQPDDFATYYCQQYNSYPW | |
| | Region | TFGQGTKVEIK | |
| | LCDR1 | RASQSISSWLA | 1254 |
| | LCDR2 | DASSLES | 1255 |
| | LCDR3 | QQYNSYPWT | 1256 |
| | LFRW1 | DIQMTQSPSTLSASVGDRVTITC | 1257 |
| | LFRW2 | WYQQKPGKAPKLLIY | 1258 |
| | LFRW3 | GVPSRFSGSGSGTEFTLTISSLQPDDFATYYC | 1259 |
| | LFRW4 | FGQGTKVEIK | 1260 |
| S144-1407 | Heavy Chain | QVQLVQSGAEVKKPGSSVKVSCKASGGTFSSYTISWVRQAPGQGLE | 1261 |
| (Spike/ | | WMGRIIPVRDIANYAQKFQGRVTITADKSTRTAYMEVSSLRSEDTAV | |
| RBD) | | YYCAATELRSDGLDIWGQGTMVTVSSASTKGPSVFPLAPSSKSTSGGT | |
| | | AALGCLVKDYFPEPVTVSWNSGALTSGVHTFPAVLQSSG | |
| | Heavy Chain | QVQLVQSGAEVKKPGSSVKVSCKASGGTFSSYTISWVRQAPGQGLE | 1262 |
| | Variable | WMGRIIPVRDIANYAQKFQGRVTITADKSTRTAYMEVSSLRSEDTAV | |
| | Region | YYCAATELRSDGLDIWGQGTMVTVSS | |
| | HCDR1 | SYTIS | 1263 |
| | HCDR2 | RIIPVRDIANYAQKFQG | 1264 |
| | HCDR3 | TELRSDGLDI | 1265 |
| | HFRW1 | QVQLVQSGAEVKKPGSSVKVSCKASGGTFS | 1266 |
| | HFRW2 | WVRQAPGQGLEWMG | 1267 |
| | HFRW3 | RVTITADKSTRTAYMEVSSLRSEDTAVYYCAA | 1268 |
| | HFRW4 | WGQGTMVTVSS | 1269 |
| | Light Chain | DIQMTQSPSTLSASVGDRVTITCRASQSISSWLAWYQQKPGKAPKLLI | 1270 |
| | | YDASSLESGVPSRFSGSGSGTEFTLTVSSLQPDDFATYYCQQYNNYSPI | |
| | | TFGQGTKLEIKRTVAAPSVFIFPPSDEQLKSGTASVVCLLNNFYPREAK | |
| | | VQWKVDNALQSGNSQESVTEQDSKDSTYSLSSTLTLSKADYE | |
| | Light Chain | DIQMTQSPSTLSASVGDRVTITCRASQSISSWLAWYQQKPGKAPKLLI | 1271 |
| | Variable | YDASSLESGVPSRFSGSGSGTEFTLTVSSLQPDDFATYYCQQYNNYSPI | |
| | Region | TFGQGTKLEIK | |
| | LCDR1 | RASQSISSWLA | 1272 |
| | LCDR2 | DASSLES | 1273 |
| | LCDR3 | QQYNNYSPIT | 1274 |
| | LFRW1 | DIQMTQSPSTLSASVGDRVTITC | 1275 |
| | LFRW2 | WYQQKPGKAPKLLIY | 1276 |
| | LFRW3 | GVPSRFSGSGSGTEFTLTVSSLQPDDFATYYC | 1277 |
| | LFRW4 | FGQGTKLEIK | 1278 |
| S144-1569 | Heavy Chain | QVQLVQSGAEVKKPGASVKVSCKASGYTFSNYGISWVRQAPGQGLE | 1279 |
| (ORF8) | | WMGWISAYNGNTKYPQKLQGRVTMSTDTSTSTAYMELRSLRSDDTA | |
| | | VYYCARETRYGMDVWGQGTTVTVSSASTKGPSVFPLAPSSKSTSGGT | |
| | | AALGCLVKDYFPEPVTVSWNSGALTSGVHTFPAVLQSSG | |
| | Heavy Chain | QVQLVQSGAEVKKPGASVKVSCKASGYTFSNYGISWVRQAPGQGLE | 1280 |
| | Variable | WMGWISAYNGNTKYPQKLQGRVTMSTDTSTSTAYMELRSLRSDDTA | |
| | Region | VYYCARETRYGMDVWGQGTTVTVSS | |
| | HCDR1 | NYGIS | 1281 |
| | HCDR2 | WISAYNGNTKYPQKLQG | 1282 |
| | HCDR3 | ETRYGMDV | 1283 |
| | HFRW1 | QVQLVQSGAEVKKPGASVKVSCKASGYTFS | 1284 |
| | HFRW2 | WVRQAPGQGLEWMG | 1285 |
| | HFRW3 | RVTMSTDTSTSTAYMELRSLRSDDTAVYYCAR | 1286 |
| | HFRW4 | WGQGTTVTVSS | 1287 |
| | Light Chain | QPVLTQPPSASASLGASVTLTCTLSSGYSNYKVDWYQQRPGKGPQFV | 1288 |
| | | MRVGTGGIVGSKGDGIPDRFSVLGSGLNRYLTIKNIQEEDESDYHCGA | |
| | | DHGSGSNFVRVFGGGTKLTVLGQPKAAPSVTLFPPSSEELQANKATL | |
| | | VCLISDFYPGAVTVAWKADSSPVKAGVETTTPSKQSNNKYAASSYLS | |
| | | LTPEQWKSH | |
| | Light Chain | QPVLTQPPSASASLGASVTLTCTLSSGYSNYKVDWYQQRPGKGPQFV | 1289 |
| | Variable | MRVGTGGIVGSKGDGIPDRFSVLGSGLNRYLTIKNIQEEDESDYHCGA | |
| | Region | DHGSGSNFVRVFGGGTKLTVL | |
| | LCDR1 | TLSSGYSNYKVD | 1290 |
| | LCDR2 | VGTGGIVGSKGD | 1291 |
| | LCDR3 | GADHGSGSNFVRV | 1292 |
| | LFRW1 | QPVLTQPPSASASLGASVTLTC | 1293 |
| | LFRW2 | WYQQRPGKGPQFVMR | 1294 |
| | LFRW3 | GIPDRFSVLGSGLNRYLTIKNIQEEDESDYHC | 1295 |
| | LFRW4 | FGGGTKLTVL | 1296 |
| S144-1641 | Heavy Chain | EVQLVQSGAEVKKPGESLKISCKGSGYTFTSYWIGWVRQMPGKGLE | 1297 |
| (Spike/ | | WMGIIYLGDSDTRYSPSFQGQVTISADKSISTAYLQWNSLKASDTAM | |
| RBD) | | YYCARQVTGTTSWFDPWGQGTLVTVSSASTKGPSVFPLAPSSKSTSG | |
| | | GTAALGCLVKDYFPEPVTVSWNSGALTSGVHTFPAVLQSSG | |
| | Heavy Chain | EVQLVQSGAEVKKPGESLKISCKGSGYTFTSYWIGWVRQMPGKGLE | 1298 |
| | Variable | WMGIIYLGDSDTRYSPSFQGQVTISADKSISTAYLQWNSLKASDTAM | |
| | Region | YYCARQVTGTTSWFDPWGQGTLVTVSS | |
| | HCDR1 | SYWIG | 1299 |
| | HCDR2 | IIYLGDSDTRYSPSFQG | 1300 |
| | HCDR3 | QVTGTTSWFDP | 1301 |
| | HFRW1 | EVQLVQSGAEVKKPGESLKISCKGSGYTFT | 1302 |
| | HFRW2 | WVRQMPGKGLEWMG | 1303 |
| | HFRW3 | QVTISADKSISTAYLQWNSLKASDTAMYYCAR | 1304 |
| | HFRW4 | WGQGTLVTVSS | 1305 |
| | Light Chain | DIQMTQSPSTLSASVGERVTITCRASQSISRWLAWYQQKPGKAPKLLI | 1306 |
| | | YDASSLESGVPSRFSGSGSGTEFTLTISSLQPDDFATYHCHQYSTYSLT | |
| | | FGGGTKVDIKRTVAAPSVFIFPPSDEQLKSGTASVVCLLNNFYPREAK | |
| | | VQWKVDNALQSGNSQESVTEQDSKDSTYSLSSTLTLSKADYE | |
| | Light Chain | DIQMTQSPSTLSASVGERVTITCRASQSISRWLAWYQQKPGKAPKLLI | 1307 |
| | Variable | YDASSLESGVPSRFSGSGSGTEFTLTISSLQPDDFATYHCHQYSTYSLT | |
| | Region | FGGGTKVDIK | |
| | LCDR1 | RASQSISRWLA | 1308 |
| | LCDR2 | DASSLES | 1309 |
| | LCDR3 | HQYSTYSLT | 1310 |
| | LFRW1 | DIQMTQSPSTLSASVGERVTITC | 1311 |
| | LFRW2 | WYQQKPGKAPKLLIY | 1312 |
| | LFRW3 | GVPSRFSGSGSGTEFTLTISSLQPDDFATYHC | 1313 |
| | LFRW4 | FGGGTKVDIK | 1314 |
| S144-1827 | Heavy Chain | EVQLVESGGDVVQPGGSLRLSCAASGITFSNYWMTWVRQAPGKGLE | 1315 |
| (Spike/ | | WVATIKKDGGEQYYVDSVKGRFTISRDNARNSLYLQINSLRAEDTAV | |
| RBD) | | YYCARGGSSSSYYWIYWGQGTLVTVSSGSASAPTLFPLVSCENSPSDT | |
| | | SSV | |
| | Heavy Chain | EVQLVESGGDVVQPGGSLRLSCAASGITFSNYWMTWVRQAPGKGLE | 1316 |
| | Variable | WVATIKKDGGEQYYVDSVKGRFTISRDNARNSLYLQINSLRAEDTAV | |
| | Region | YYCARGGSSSSYYWIYWGQGTLVTVSS | |
| | HCDR1 | NYWMT | 1317 |
| | HCDR2 | TIKKDGGEQYYVDSVKG | 1318 |
| | HCDR3 | GGSSSSYYWIY | 1319 |
| | HFRW1 | EVQLVESGGDVVQPGGSLRLSCAASGITFS | 1320 |
| | HFRW2 | WVRQAPGKGLEWVA | 1321 |
| | HFRW3 | RFTISRDNARNSLYLQINSLRAEDTAVYYCAR | 1322 |
| | HFRW4 | WGQGTLVTVSS | 1323 |
| | Light Chain | EIVLTQSPGTLSLSPGERATLSCRASQSISNSYLVWYQQKPGQAPRLLI | 1324 |
| | | YGASTRATGIPDRFSGSGSGTDFTLTISRLEPEDFAVYYCQQYGSSPW | |
| | | TFGQGTTVEIKRTVAAPSVFIFPPSDEQLKSGTASVVCLLNNFYPREAK | |
| | | VQWKVDNALQSGNSQESVTEQDSKDSTYSLSSTLTLSKADYE | |
| | Light Chain | EIVLTQSPGTLSLSPGERATLSCRASQSISNSYLVWYQQKPGQAPRLLI | 1325 |
| | Variable | YGASTRATGIPDRFSGSGSGTDFTLTISRLEPEDFAVYYCQQYGSSPW | |
| | Region | TFGQGTTVEIK | |
| | LCDR1 | RASQSISNSYLV | 1326 |
| | LCDR2 | GASTRAT | 1327 |
| | LCDR3 | QQYGSSPWT | 1328 |
| | LFRW1 | EIVLTQSPGTLSLSPGERATLSC | 1329 |
| | LFRW2 | WYQQKPGQAPRLLIY | 1330 |
| | LFRW3 | GIPDRFSGSGSGTDFTLTISRLEPEDFAVYYC | 1331 |
| | LFRW4 | FGQGTTVEIK | 1332 |
| S144-1848 | Heavy Chain | EVQLVESGGGLVKPGGSLRLSCAASGFTFSSYSMNWVRQAPGKGLE | 1333 |
| (NP) | | WVSSISSSSSYIYYADSVKGRFTISRDNAKNSLYLQLNSLRAEDTAVY | |
| | | YCARDRDQLIFSAAFDIWGQGTMVTVSSASTKGPSVFPLAPSSKSTSG | |
| | | GTAALGCLVKDYFPEPVTVSWNSGALTSGVHTFPAVLQSSG | |
| | Heavy Chain | EVQLVESGGGLVKPGGSLRLSCAASGFTFSSYSMNWVRQAPGKGLE | 1334 |
| | Variable | WVSSISSSSSYIYYADSVKGRFTISRDNAKNSLYLQLNSLRAEDTAVY | |
| | Region | YCARDRDQLIFSAAFDIWGQGTMVTVSS | |
| | HCDR1 | SYSMN | 1335 |
| | HCDR2 | SISSSSSYIYYADSVKG | 1336 |
| | HCDR3 | DRDQLIFSAAFDI | 1337 |
| | HFRW1 | EVQLVESGGGLVKPGGSLRLSCAASGFTFS | 1338 |
| | HFRW2 | WVRQAPGKGLEWVS | 1339 |
| | HFRW3 | RFTISRDNAKNSLYLQLNSLRAEDTAVYYCAR | 1340 |
| | HFRW4 | WGQGTMVTVSS | 1341 |
| | Light Chain | QSVLTQPPSASGTPGQRVTISCSGSSSNIEHNYVFWYQQLPGTAPKLLI | 1342 |
| | | YSNNHRPSGVPDRFSGSKSGTSASLAISGLRSEDEADYYCAAWDASLS | |
| | | GPVVFAGGTKLTVLGQPKAAPSVTLFPPSSEELQANKATLVCLISDFY | |
| | | PGAVTVAWKADSSPVKAGVETTTPSKQSNNKYAASS | |
| | Light Chain | QSVLTQPPSASGTPGQRVTISCSGSSSNIEHNYVFWYQQLPGTAPKLLI | 1343 |
| | Variable | YSNNHRPSGVPDRFSGSKSGTSASLAISGLRSEDEADYYCAAWDASLS | |
| | Region | GPVVFAGGTKLTVL | |
| | LCDR1 | SGSSSNIEHNYVF | 1344 |
| | LCDR2 | SNNHRPS | 1345 |
| | LCDR3 | AAWDASLSGPVV | 1346 |
| | LFRW1 | QSVLTQPPSASGTPGQRVTISC | 1347 |
| | LFRW2 | WYQQLPGTAPKLLIY | 1348 |
| | LFRW3 | GVPDRFSGSKSGTSASLAISGLRSEDEADYYC | 1349 |
| | LFRW4 | FAGGTKLTVL | 1350 |
| S144-1850 | Heavy Chain | EVQLVESGGGLVQPGGSLRLSCAASGFTFSSYAMSWVRQAPGKGLE | 1351 |
| (Spike/ | | WVSAISGSGGSTYYADSVKGRFTISRANSKNTLYLQMNSLRAEDTAV | |
| RBD) | | YYCAKGPRFSRDYFDYWGQGTLVTVSSASTKGPSVFPLAPSSKSTSG | |
| | | GTAALGCLVKDYFPEPVTVSWNSGALTSGVHTFPAVLQSSG | |
| | Heavy Chain | EVQLVESGGGLVQPGGSLRLSCAASGFTFSSYAMSWVRQAPGKGLE | 1352 |
| | Variable | WVSAISGSGGSTYYADSVKGRFTISRANSKNTLYLQMNSLRAEDTAV | |
| | Region | YYCAKGPRFSRDYFDYWGQGTLVTVSS | |
| | HCDR1 | SYAMS | 1353 |
| | HCDR2 | AISGSGGSTYYADSVKG | 1354 |
| | HCDR3 | GPRFSRDYFDY | 1355 |
| | HFRW1 | EVQLVESGGGLVQPGGSLRLSCAASGFTFS | 1356 |
| | HFRW2 | WVRQAPGKGLEWVS | 1357 |
| | HFRW3 | RFTISRANSKNTLYLQMNSLRAEDTAVYYCAK | 1358 |
| | HFRW4 | WGQGTLVTVSS | 1359 |
| | Light Chain | DIQMTQSPSTLSASVGDRVTITCRASQSITSWLAWYQQKPGKAPKLLI | 1360 |
| | | YDASNLESGVPSRFSGSGSGTEFTLTISSLQPDDFATYYCQQYNNYLG | |
| | | TFGQGTKVEIKRTVAAPSVFIFPPSDEQLKSGTASVVCLLNNFYPREA | |
| | | KVQWKVDNALQSGNSQESVTEQDSKDSTYSLSSTLTLSKADYE | |
| | Light Chain | DIQMTQSPSTLSASVGDRVTITCRASQSITSWLAWYQQKPGKAPKLLI | 1361 |
| | Variable | YDASNLESGVPSRFSGSGSGTEFTLTISSLQPDDFATYYCQQYNNYLG | |
| | Region | TFGQGTKVEIK | |
| | LCDR1 | RASQSITSWLA | 1362 |
| | LCDR2 | DASNLES | 1363 |
| | LCDR3 | QQYNNYLGT | 1364 |
| | LFRW1 | DIQMTQSPSTLSASVGDRVTITC | 1365 |
| | LFRW2 | WYQQKPGKAPKLLIY | 1366 |
| | LFRW3 | GVPSRFSGSGSGTEFTLTISSLQPDDFATYYC | 1367 |
| | LFRW4 | FGQGTKVEIK | 1368 |
| S144-2234 | Heavy Chain | QVQLVQSGAEVKKPGSSVKVSCKASGGTFSRYTISWVRQAPGQGLE | 1369 |
| (ORF8) | | WMGRIIPILGTANYAQNFQGRVTITADKSTSTAYMELSSLRSEDTAVY | |
| | | YCARHGYSYGPFDYWGQGTLVTVSSASTKGPSVFPLAPSSKSTSGGT | |
| | | AALGCLVKDYFPEPVTVSWNSGALTSGVHTFPAVLQSSG | |
| | Heavy Chain | QVQLVQSGAEVKKPGSSVKVSCKASGGTFSRYTISWVRQAPGQGLE | 1370 |
| | Variable | WMGRIIPILGTANYAQNFQGRVTITADKSTSTAYMELSSLRSEDTAVY | |
| | Region | YCARHGYSYGPFDYWGQGTLVTVSS | |
| | HCDR1 | RYTIS | 1371 |
| | HCDR2 | RIIPILGTANYAQNFQG | 1372 |
| | HCDR3 | HGYSYGPFDY | 1373 |
| | HFRW1 | QVQLVQSGAEVKKPGSSVKVSCKASGGTFS | 1374 |
| | HFRW2 | WVRQAPGQGLEWMG | 1375 |
| | HFRW3 | RVTITADKSTSTAYMELSSLRSEDTAVYYCAR | 1376 |
| | HFRW4 | WGQGTLVTVSS | 1377 |
| | Light Chain | DIVMTQSPDSLTVSLGERATINCKSSQSVLYSSNNKNYLAWYQQKPG | 1378 |
| | | QPPKLLIYWASTRESGVPDRFSGSGSGTDFTLTVSSLQAEDVAVYYCQ | |
| | | QYYSTPGTFGQGTKVEIKRTVAAPSVFIFPPSDEQLKSGTASVVCLLN | |
| | | NFYPREAKVQWKVDNALQSGNSQESVTEQDSKDSTYSLSSTLTLSKA | |
| | | DYE | |
| | Light Chain | DIVMTQSPDSLTVSLGERATINCKSSQSVLYSSNNKNYLAWYQQKPG | 1379 |
| | Variable | QPPKLLIYWASTRESGVPDRFSGSGSGTDFTLTVSSLQAEDVAVYYCQ | |
| | Region | QYYSTPGTFGQGTKVEIK | |
| | LCDR1 | KSSQSVLYSSNNKNYLA | 1380 |
| | LCDR2 | WASTRES | 1381 |
| | LCDR3 | QQYYSTPGT | 1382 |
| | LFRW1 | DIVMTQSPDSLTVSLGERATINC | 1383 |
| | LFRW2 | WYQQKPGQPPKLLIY | 1384 |
| | LFRW3 | GVPDRFSGSGSGTDFTLTVSSLQAEDVAVYYC | 1385 |
| | LFRW4 | FGQGTKVEIK | 1386 |
| S564-105 | Heavy Chain | QVRLQESGPGLVKPSQTLSLTCTVSGGSISSGSYYWSWIRQPAGKGLE | 1387 |
| (NP) | | WIGRFHTSGSTNYNPSLKSRVTISVDTSKNQFSLKLSSVTAADTAVYY | |
| | | CARDLKGKTWIQTPFDYWGQGILVTVSSASTKGPSVFPLAPSSKSTSG | |
| | | GTAALGCLVKDYFPEPVTVSWNSGALTSGVHTFPAVLQSSG | |
| | Heavy Chain | QVRLQESGPGLVKPSQTLSLTCTVSGGSISSGSYYWSWIRQPAGKGLE | 1388 |
| | Variable | WIGRFHTSGSTNYNPSLKSRVTISVDTSKNQFSLKLSSVTAADTAVYY | |
| | Region | CARDLKGKTWIQTPFDYWGQGILVTVSS | |
| | HCDR1 | SGSYYWS | 1389 |
| | HCDR2 | RFHTSGSTNYNPSLKS | 1390 |
| | HCDR3 | DLKGKTWIQTPFDY | 1391 |
| | HFRW1 | QVRLQESGPGLVKPSQTLSLTCTVSGGSIS | 1392 |
| | HFRW2 | WIRQPAGKGLEWIG | 1393 |
| | HFRW3 | RVTISVDTSKNQFSLKLSSVTAADTAVYYCAR | 1394 |
| | HFRW4 | WGQGILVTVSS | 1395 |
| | Light Chain | QSALTQPASVSGSPGQSITISCTGTSSDVGAYNYVSWYQQHPGKAPKL | 1396 |
| | | MIYEVSNRPSGVSNRFSGSKSGNTASLTISGLQAEDEADYYCSSYTSST | |
| | | FFGTGTTVTVLGQPKANPTVTLFPPSSEELQANKATLVCLISDFYPGA | |
| | | VTVAWKADGSPVKAGVETTTPSKQSNNKYAASSY | |
| | Light Chain | QSALTQPASVSGSPGQSITISCTGTSSDVGAYNYVSWYQQHPGKAPKL | 1397 |
| | Variable | MIYEVSNRPSGVSNRFSGSKSGNTASLTISGLQAEDEADYYCSSYTSST | |
| | Region | FFGTGTTVTVL | |
| | LCDR1 | TGTSSDVGAYNYVS | 1398 |
| | LCDR2 | EVSNRPS | 1399 |
| | LCDR3 | SSYTSSTF | 1400 |
| | LFRW1 | QSALTQPASVSGSPGQSITISC | 1401 |
| | LFRW2 | WYQQHPGKAPKLMIY | 1402 |
| | LFRW3 | GVSNRFSGSKSGNTASLTISGLQAEDEADYYC | 1403 |
| | LFRW4 | FGTGTTVTVL | 1404 |
| S564-14 | Heavy Chain | EVQLVESGGGLVQPGGSLRLSCAASGLTFSSYWMSWARQAPGKGLE | 1405 |
| (Spike/ | | WVANIKKDGSEKYYVDSVKGRFTISRDNAKNSLYLQMNSLRVEDTA | |
| RBD) | | VYYCASEPPHYGGNSGAEYFQHWGQGTLVTVSSAPTKAPDVFPIISG | |
| | | CRHPKDNSPVVLACLITGYH | |
| | Heavy Chain | EVQLVESGGGLVQPGGSLRLSCAASGLTFSSYWMSWARQAPGKGLE | 1406 |
| | Variable | WVANIKKDGSEKYYVDSVKGRFTISRDNAKNSLYLQMNSLRVEDTA | |
| | Region | VYYCASEPPHYGGNSGAEYFQHWGQGTLVTVSS | |
| | HCDR1 | SYWMS | 1407 |
| | HCDR2 | NIKKDGSEKYYVDSVKG | 1408 |
| | HCDR3 | EPPHYGGNSGAEYFQH | 1409 |
| | HFRW1 | EVQLVESGGGLVQPGGSLRLSCAASGLTFS | 1410 |
| | HFRW2 | WARQAPGKGLEWVA | 1411 |
| | HFRW3 | RFTISRDNAKNSLYLQMNSLRVEDTAVYYCAS | 1412 |
| | HFRW4 | WGQGTLVTVSS | 1413 |
| | Light Chain | SYVLTQPPSVSVAPGKTARITCGGNNIGSKSVHWYQQRPGQAPVLVI | 1414 |
| | | YYDSDRPSGIPERFSGSNSGNTATLTISRVEAGDEADYYCQVWDSSSD | |
| | | HHYVFGTGTKVTVLGQPKANPTVTLFPPSSEELQANKATLVCLISDFY | |
| | | PGAVTVAWKADSSPVKAGVETTKPSKQSNNKYAASS | |
| | Light Chain | SYVLTQPPSVSVAPGKTARITCGGNNIGSKSVHWYQQRPGQAPVLVI | 1415 |
| | Variable | YYDSDRPSGIPERFSGSNSGNTATLTISRVEAGDEADYYCQVWDSSSD | |
| | Region | HHYVFGTGTKVTVL | |
| | LCDR1 | GGNNIGSKSVH | 1416 |
| | LCDR2 | YDSDRPS | 1417 |
| | LCDR3 | QVWDSSSDHHYV | 1418 |
| | LFRW1 | SYVLTQPPSVSVAPGKTARITC | 1419 |
| | LFRW2 | WYQQRPGQAPVLVIY | 1420 |
| | LFRW3 | GIPERFSGSNSGNTATLTISRVEAGDEADYYC | 1421 |
| | LFRW4 | FGTGTKVTVL | 1422 |
| S564-68 | Heavy Chain | QVQLVQSGAEVKKPGASVKVSCKASGYIFTGYYMHWVRQAPGQGL | 1423 |
| (Spike/ | | EWMGWINPNSGGTNYAQKFQGRVTMTRDTSITTAYMELSRLRSDDT | |
| RBD) | | AFYYCARVKRFSIFGVELDYWGQGTLVTVSSASTKGPSVFPLAPSSKS | |
| | | TSGGTAALGCLVKDYFPEPVTVSWNSGALTSGVHTFPAVLQSSG | |
| | Heavy Chain | QVQLVQSGAEVKKPGASVKVSCKASGYIFTGYYMHWVRQAPGQGL | 1424 |
| | Variable | EWMGWINPNSGGTNYAQKFQGRVTMTRDTSITTAYMELSRLRSDDT | |
| | Region | AFYYCARVKRFSIFGVELDYWGQGTLVTVSS | |
| | HCDR1 | GYYMH | 1425 |
| | HCDR2 | WINPNSGGTNYAQKFQG | 1426 |
| | HCDR3 | VKRFSIFGVELDY | 1427 |
| | HFRW1 | QVQLVQSGAEVKKPGASVKVSCKASGYIFT | 1428 |
| | HFRW2 | WVRQAPGQGLEWMG | 1429 |
| | HFRW3 | RVTMTRDTSITTAYMELSRLRSDDTAFYYCAR | 1430 |
| | HFRW4 | WGQGTLVTVSS | 1431 |
| | Light Chain | QSALTQPPSASGSPGQSVTISCTGTSSDVGGYNYVSWYQQHPGKAPK | 1432 |
| | | LMIYEVSKRPSGVPDRFSGSKSGNTASLTVSGLQAEDEADYFCSSYAD | |
| | | SNNLVFGGGTKLTVLGQPKAAPSVTLFPPSSEELQANKATLVCLISDF | |
| | | CPGAVTVAWKADSSPVKAGVETTTPSKQSNNKYAASSY | |
| | Light Chain | QSALTQPPSASGSPGQSVTISCTGTSSDVGGYNYVSWYQQHPGKAPK | 1433 |
| | Variable | LMIYEVSKRPSGVPDRFSGSKSGNTASLTVSGLQAEDEADYFCSSYAD | |
| | Region | SNNLVFGGGTKLTVL | |
| | LCDR1 | TGTSSDVGGYNYVS | 1434 |
| | LCDR2 | EVSKRPS | 1435 |
| | LCDR3 | SSYADSNNLV | 1436 |
| | LFRW1 | QSALTQPPSASGSPGQSVTISC | 1437 |
| | LFRW2 | WYQQHPGKAPKLMIY | 1438 |
| | LFRW3 | GVPDRFSGSKSGNTASLTVSGLQAEDEADYFC | 1439 |
| | LFRW4 | FGGGTKLTVL | 1440 |
| S564-98 | Heavy Chain | QVQLQESGPGLVKPSETLSLTCTVSGGSISSYYWSWIRQPPGKGLEWI | 1441 |
| (NP) | | GYIYYSGSTNYNPSLKSRVTISVDTSKNQFSLKLSSVTAADTAVYYCA | |
| | | RHQSRWNIVATMDFDYWGQGTLVTVSSASTKGPSVFPL | |
| | Heavy Chain | QVQLQESGPGLVKPSETLSLTCTVSGGSISSYYWSWIRQPPGKGLEWI | 1442 |
| | Variable | GYIYYSGSTNYNPSLKSRVTISVDTSKNQFSLKLSSVTAADTAVYYCA | |
| | Region | RHQSRWNIVATMDFDYWGQGTLVTVSS | |
| | HCDR1 | SYYWS | 1443 |
| | HCDR2 | YIYYSGSTNYNPSLKS | 1444 |
| | HCDR3 | HQSRWNIVATMDFDY | 1445 |
| | HFRW1 | QVQLQESGPGLVKPSETLSLTCTVSGGSIS | 1446 |
| | HFRW2 | WIRQPPGKGLEWIG | 1447 |
| | HFRW3 | RVTISVDTSKNQFSLKLSSVTAADTAVYYCAR | 1448 |
| | HFRW4 | WGQGTLVTVSS | 1449 |
| | Light Chain | DIQMTQSPSSLSASVGDRVTITCRASQSIRSYLNWYQQKPGKAPKLLI | 1450 |
| | | YAASSLQSGVPSRFSGSGSGTDFTLTIGSLQPEDFATYYCQQSYSTSVA | |
| | | FGQGTKVEIKRTVAAPSVFIFPPSDEQLKSGTASVVCLLNNFYPREAK | |
| | | VQWKVDNALQSGNSQESVTEQDSKDSTYSLSSTLTLSKADYE | |
| | Light Chain | DIQMTQSPSSLSASVGDRVTITCRASQSIRSYLNWYQQKPGKAPKLLI | 1451 |
| | Variable | YAASSLQSGVPSRFSGSGSGTDFTLTIGSLQPEDFATYYCQQSYSTSVA | |
| | Region | FGQGTKVEIK | |
| | LCDR1 | RASQSIRSYLN | 1452 |
| | LCDR2 | AASSLQS | 1453 |
| | LCDR3 | QQSYSTSVA | 1454 |
| | LFRW1 | DIQMTQSPSSLSASVGDRVTITC | 1455 |
| | LFRW2 | WYQQKPGKAPKLLIY | 1456 |
| | LFRW3 | GVPSRFSGSGSGTDFTLTIGSLQPEDFATYYC | 1457 |
| | LFRW4 | FGQGTKVEIK | 1458 |
| S564-105 | Heavy Chain | QVRLQESGPGLVKPSQTLSLTCTVSGGSISSGSYYWSWIRQPAGKGLE | 1459 |
| (NP) | | WIGRFHTSGSTNYNPSLKSRVTISVDTSKNQFSLKLSSVTAADTAVYY | |
| | | CARDLKGKTWIQTPFDYWGQGILVTVSSASTKGPSVFPLAPSSKSTSG | |
| | | GTAALGCLVKDYFPEPVTVSWNSGALTSGVHTFPAVLQSSG | |
| | Heavy Chain | QVRLQESGPGLVKPSQTLSLTCTVSGGSISSGSYYWSWIRQPAGKGLE | 1460 |
| | Variable | WIGRFHTSGSTNYNPSLKSRVTISVDTSKNQFSLKLSSVTAADTAVYY | |
| | Region | CARDLKGKTWIQTPFDYWGQGILVTVSS | |
| | HCDR1 | SGSYYWS | 1461 |
| | HCDR2 | RFHTSGSTNYNPSLKS | 1462 |
| | HCDR3 | DLKGKTWIQTPFDY | 1463 |
| | HFRW1 | QVRLQESGPGLVKPSQTLSLTCTVSGGSIS | 1464 |
| | HFRW2 | WIRQPAGKGLEWIG | 1465 |
| | HFRW3 | RVTISVDTSKNQFSLKLSSVTAADTAVYYCAR | 1466 |
| | HFRW4 | WGQGILVTVSS | 1467 |
| | Light Chain | QSALTQPASVSGSPGQSITISCTGTSSDVGAYNYVSWYQQHPGKAPKL | 1468 |
| | | MIYEVSNRPSGVSNRFSGSKSGNTASLTISGLQAEDEADYYCSSYTSST | |
| | | FFGTGTTVTVLGQPKANPTVTLFPPSSEELQANKATLVCLISDFYPGA | |
| | | VTVAWKADGSPVKAGVETTTPSKQSNNKYAASSY | |
| | Light Chain | QSALTQPASVSGSPGQSITISCTGTSSDVGAYNYVSWYQQHPGKAPKL | 1469 |
| | Variable | MIYEVSNRPSGVSNRFSGSKSGNTASLTISGLQAEDEADYYCSSYTSST | |
| | Region | FFGTGTTVTVL | |
| | LCDR1 | TGTSSDVGAYNYVS | 1470 |
| | LCDR2 | EVSNRPS | 1471 |
| | LCDR3 | SSYTSSTF | 1472 |
| | LFRW1 | QSALTQPASVSGSPGQSITISC | 1473 |
| | LFRW2 | WYQQHPGKAPKLMIY | 1474 |
| | LFRW3 | GVSNRFSGSKSGNTASLTISGLQAEDEADYYC | 1475 |
| | LFRW4 | FGTGTTVTVL | 1476 |
| S564-134 | Heavy Chain | QVQLVQSGAEVKKPGASVKVSCKASGYTFTGYYMHWVRQAPGQGL | 1477 |
| (Spike/ | | EWMGWINPNSGGTNYAQKFQGRVTMTRDTSINTAYMELSRLRSDDT | |
| RBD) | | AVYYCTRVGRFSIFGVELDYWGQGTLVTVSSASTKGPSVFPLAPSSKS | |
| | | TSGGTAALGCLVKDYFPEPVTVSWNSGALTSGVHTFPAVLQSSG | |
| | Heavy Chain | QVQLVQSGAEVKKPGASVKVSCKASGYTFTGYYMHWVRQAPGQGL | 1478 |
| | Variable | EWMGWINPNSGGTNYAQKFQGRVTMTRDTSINTAYMELSRLRSDDT | |
| | Region | AVYYCTRVGRFSIFGVELDYWGQGTLVTVSS | |
| | HCDR1 | GYYMH | 1479 |
| | HCDR2 | WINPNSGGTNYAQKFQG | 1480 |
| | HCDR3 | VGRFSIFGVELDY | 1481 |
| | HFRW1 | QVQLVQSGAEVKKPGASVKVSCKASGYTFT | 1482 |
| | HFRW2 | WVRQAPGQGLEWMG | 1483 |
| | HFRW3 | RVTMTRDTSINTAYMELSRLRSDDTAVYYCTR | 1484 |
| | HFRW4 | WGQGTLVTVSS | 1485 |
| | Light Chain | QSALTQPPSASGSPGQSVTISCTGTSSDVGGYNYVSWYQQHPGKAPK | 1486 |
| | | LMIYEVNKRPSGVPDRFSGSKSGNTASLTVSGLQADDEADYYCSSYA | |
| | | GSNNLVFGGGTKLTVLGQPKAAPSVTLFPPSSEELQANKATLVCLISD | |
| | | FYPGAVTVAWKADSSPVKAGVETTTPSKQSNNKYAASS | |
| | Light Chain | QSALTQPPSASGSPGQSVTISCTGTSSDVGGYNYVSWYQQHPGKAPK | 1487 |
| | Variable | LMIYEVNKRPSGVPDRFSGSKSGNTASLTVSGLQADDEADYYCSSYA | |
| | Region | GSNNLVFGGGTKLTVL | |
| | LCDR1 | TGTSSDVGGYNYVS | 1488 |
| | LCDR2 | EVNKRPS | 1489 |
| | LCDR3 | SSYAGSNNLV | 1490 |
| | LFRW1 | QSALTQPPSASGSPGQSVTISC | 1491 |
| | LFRW2 | WYQQHPGKAPKLMIY | 1492 |
| | LFRW3 | GVPDRFSGSKSGNTASLTVSGLQADDEADYYC | 1493 |
| | LFRW4 | FGGGTKLTVL | 1494 |
| S564-138 | Heavy Chain | QVLLVQSGAEVKKPGASVKVSCKASGYTFTGYYLHWVRQAPGQGLE | 1495 |
| (Spike/ | | WMGWINPISGGTNYAQNFQDRVTMTRDTSIITAYMELSRLRSDDTAV | |
| RBD) | | YYCARLAYYYDSSAYRGAFDIWGQGTMVTVSSASTKGPSVFPLAPSS | |
| | | KSTSGGTAALGCLVKDYFPEPVTVSWNSGALTSGVHTFPAVLQSS | |
| | Heavy Chain | QVLLVQSGAEVKKPGASVKVSCKASGYTFTGYYLHWVRQAPGQGLE | 1496 |
| | Variable | WMGWINPISGGTNYAQNFQDRVTMTRDTSIITAYMELSRLRSDDTAV | |
| | Region | YYCARLAYYYDSSAYRGAFDIWGQGTMVTVSS | |
| | HCDR1 | GYYLH | 1497 |
| | HCDR2 | WINPISGGTNYAQNFQD | 1498 |
| | HCDR3 | LAYYYDSSAYRGAFDI | 1499 |
| | HFRW1 | QVLLVQSGAEVKKPGASVKVSCKASGYTFT | 1500 |
| | HFRW2 | WVRQAPGQGLEWMG | 1501 |
| | HFRW3 | RVTMTRDTSIITAYMELSRLRSDDTAVYYCAR | 1502 |
| | HFRW4 | WGQGTMVTVSS | 1503 |
| | Light Chain | QSALTQPASVSGSPGQSITISCTGTSSDVGGYNYVSWYQQHPGKAPKL | 1504 |
| | | MIYEVSNRPSGVSDRFSGSKSGNTASLTISGLQAEDEADYYCSSYTSSS | |
| | | TYVFGTGTKVTVLGQPKANPTVTLFPPSSEELQANKATLVCLISDFYP | |
| | | GAVTVAWKADGSPVKAGVETTKPSKQSNNKYAASS | |
| | Light Chain | QSALTQPASVSGSPGQSITISCTGTSSDVGGYNYVSWYQQHPGKAPKL | 1505 |
| | Variable | MIYEVSNRPSGVSDRFSGSKSGNTASLTISGLQAEDEADYYCSSYTSSS | |
| | Region | TYVFGTGTKVTVL | |
| | LCDR1 | TGTSSDVGGYNYVS | 1506 |
| | LCDR2 | EVSNRPS | 1507 |
| | LCDR3 | SSYTSSSTYV | 1508 |
| | LFRW1 | QSALTQPASVSGSPGQSITISC | 1509 |
| | LFRW2 | WYQQHPGKAPKLMIY | 1510 |
| | LFRW3 | GVSDRFSGSKSGNTASLTISGLQAEDEADYYC | 1511 |
| | LFRW4 | FGTGTKVTVL | 1512 |
| S564-152 | Heavy Chain | QVQLVESGGGVVQPGRSLRLSCAASGFTFSYYGMHWVRQAPGKGLE | 1513 |
| (Spike/ | | WVAVIWYDGSNKHYADSVKGRFTISRDNSKNTLYLQMNSLRAEDTA | |
| RBD) | | VYYCAKNAAPYCSGGSCYGTYFDYWGQGTLVTVSSASTKGPSVFPL | |
| | | APSSKSTSGGTAALGCLVKDYFPEPVTVSWNSGALTSGVHTFPAVLQ | |
| | | SSG | |
| | Heavy Chain | QVQLVESGGGVVQPGRSLRLSCAASGFTFSYYGMHWVRQAPGKGLE | 1514 |
| | Variable | WVAVIWYDGSNKHYADSVKGRFTISRDNSKNTLYLQMNSLRAEDTA | |
| | Region | VYYCAKNAAPYCSGGSCYGTYFDYWGQGTLVTVSS | |
| | HCDR1 | YYGMH | 1515 |
| | HCDR2 | VIWYDGSNKHYADSVKG | 1516 |
| | HCDR3 | NAAPYCSGGSCYGTYFDY | 1517 |
| | HFRW1 | QVQLVESGGGVVQPGRSLRLSCAASGFTFS | 1518 |
| | HFRW2 | WVRQAPGKGLEWVA | 1519 |
| | HFRW3 | RFTISRDNSKNTLYLQMNSLRAEDTAVYYCAK | 1520 |
| | HFRW4 | WGQGTLVTVSS | 1521 |
| | Light Chain | DIQMTQSPSSLSASVGDRVTITCQASQDINNYLNWYQQKPGKAPKLLI | 1522 |
| | | YDASNLETGVPSRFSGSGSGTDFTFTISSLQPEDIATYYCQQYDNVPPH | |
| | | TFGQGTKLEIKRTVAAPSVFIFPPSDEQLKSGTASVVCLLNNFYPREAK | |
| | | VQWKVDNALQSGNSQESVTEQDSKDSTYSLSSTLTLSKADYE | |
| | Light Chain | DIQMTQSPSSLSASVGDRVTITCQASQDINNYLNWYQQKPGKAPKLLI | 1523 |
| | Variable | YDASNLETGVPSRFSGSGSGTDFTFTISSLQPEDIATYYCQQYDNVPPH | |
| | Region | TFGQGTKLEIK | |
| | LCDR1 | QASQDINNYLN | 1524 |
| | LCDR2 | DASNLET | 1525 |
| | LCDR3 | QQYDNVPPHT | 1526 |
| | LFRW1 | DIQMTQSPSSLSASVGDRVTITC | 1527 |
| | LFRW2 | WYQQKPGKAPKLLIY | 1528 |
| | LFRW3 | GVPSRFSGSGSGTDFTFTISSLQPEDIATYYC | 1529 |
| | LFRW4 | FGQGTKLEIK | 1530 |
| S564-218 | Heavy Chain | QVQLVQSGAEVKKPGSSVKVSCKASGGTFSSYAISWVRQAPGQGLE | 1531 |
| (Spike/ | | WMGGIIPIFGTAKYAQKFQGRVTITADESTSTAYMELSSLRSEDTAVY | |
| RBD) | | YCARGKDGYNPWGAFDIWGQGTMVTVSSGSASAPTLFPLVSCENSPS | |
| | | DTSSV | |
| | Heavy Chain | QVQLVQSGAEVKKPGSSVKVSCKASGGTFSSYAISWVRQAPGQGLE | 1532 |
| | Variable | WMGGIIPIFGTAKYAQKFQGRVTITADESTSTAYMELSSLRSEDTAVY | |
| | Region | YCARGKDGYNPWGAFDIWGQGTMVTVSS | |
| | HCDR1 | SYAIS | 1533 |
| | HCDR2 | GIIPIFGTAKYAQKFQG | 1534 |
| | HCDR3 | GKDGYNPWGAFDI | 1535 |
| | HFRW1 | QVQLVQSGAEVKKPGSSVKVSCKASGGTFS | 1536 |
| | HFRW2 | WVRQAPGQGLEWMG | 1537 |
| | HFRW3 | RVTITADESTSTAYMELSSLRSEDTAVYYCAR | 1538 |
| | HFRW4 | WGQGTMVTVSS | 1539 |
| | Light Chain | QSALTQPPSASGSPGQSVTISCTGTSSDVGGYNYVSWYQQHPGKAPK | 1540 |
| | | LMIYEVSKRPSGVPDRFSGSKSGNTASLTVSGLQAEDEADYYCSSYA | |
| | | GSNNFGVFGGGTKLTVLGQPKAAPSVTLFPPSSEELQANKATLVCLIS | |
| | | DFYPGAVTVAWKADSSPVKAGVETTTPSKQSNNKYAASSYLSLTPEQ | |
| | | WKSH | |
| | Light Chain | QSALTQPPSASGSPGQSVTISCTGTSSDVGGYNYVSWYQQHPGKAPK | 1541 |
| | Variable | LMIYEVSKRPSGVPDRFSGSKSGNTASLTVSGLQAEDEADYYCSSYA | |
| | Region | GSNNFGVFGGGTKLTVL | |
| | LCDR1 | TGTSSDVGGYNYVS | 1542 |
| | LCDR2 | EVSKRPS | 1543 |
| | LCDR3 | SSYAGSNNFGV | 1544 |
| | LFRW1 | QSALTQPPSASGSPGQSVTISC | 1545 |
| | LFRW2 | WYQQHPGKAPKLMIY | 1546 |
| | LFRW3 | GVPDRFSGSKSGNTASLTVSGLQAEDEADYYC | 1547 |
| | LFRW4 | FGGGTKLTVL | 1548 |
| S564-249 | Heavy Chain | EVQLVESGGGLVQPGGSLRLSCVASGFTFSDYAMHWVRQAPGKGLE | 1549 |
| (NP) | | YIAAISSNGGRTYYADSVKDKFTISRDNSKNILYLHMGSLRAEDTAVY | |
| | | FCARDPQSWVTSTTAHFQHWGQGTLVTVSSASPTSPKVFPLSLCSTQP | |
| | | DGNVVIACLVQGFFPQEPLSVTWSESGQGVTARNF | |
| | Heavy Chain | EVQLVESGGGLVQPGGSLRLSCVASGFTFSDYAMHWVRQAPGKGLE | 1550 |
| | Variable | YIAAISSNGGRTYYADSVKDKFTISRDNSKNILYLHMGSLRAEDTAVY | |
| | Region | FCARDPQSWVTSTTAHFQHWGQGTLVTVSS | |
| | HCDR1 | DYAMH | 1551 |
| | HCDR2 | AISSNGGRTYYADSVKD | 1552 |
| | HCDR3 | DPQSWVTSTTAHFQH | 1553 |
| | HFRW1 | EVQLVESGGGLVQPGGSLRLSCVASGFTFS | 1554 |
| | HFRW2 | WVRQAPGKGLEYIA | 1555 |
| | HFRW3 | KFTISRDNSKNILYLHMGSLRAEDTAVYFCAR | 1556 |
| | HFRW4 | WGQGTLVTVSS | 1557 |
| | Light Chain | QSALTQPASVSGSPGQSITISCTGTSSDIGGYNYVSWYQQHPGKAPKLI | 1558 |
| | | ISDVSNRPSGVSSRFSGSKSGNTASLTISGLQTEDEAHYYCSSFRSGITL | |
| | | GVFGGGTKLTVLGQPKAAPSVTLFPPSSEELQANKATLVCLISDFYPG | |
| | | AVTVAWKADSSPVKAGVETTTPSKQSNNKYAASS | |
| | Light Chain | QSALTQPASVSGSPGQSITISCTGTSSDIGGYNYVSWYQQHPGKAPKLI | 1559 |
| | Variable | ISDVSNRPSGVSSRFSGSKSGNTASLTISGLQTEDEAHYYCSSFRSGITL | |
| | Region | GVFGGGTKLTVL | |
| | LCDR1 | TGTSSDIGGYNYVS | 1560 |
| | LCDR2 | DVSNRPS | 1561 |
| | LCDR3 | SSFRSGITLGV | 1562 |
| | LFRW1 | QSALTQPASVSGSPGQSITISC | 1563 |
| | LFRW2 | WYQQHPGKAPKLIIS | 1564 |
| | LFRW3 | GVSSRFSGSKSGNTASLTISGLQTEDEAHYYC | 1565 |
| | LFRW4 | FGGGTKLTVL | 1566 |
| S564-265 | Heavy Chain | QVQLVQSGAEVKKPGASVKVSCKASGYTFTGYYMHWVRQAPGQGL | 1567 |
| (Spike/ | | EWMGWINPNSGAINYAQKFQGRVTMTRDTSISTAYMELSSLRSDDTA | |
| RBD) | | VYYCARVGRFSIFGVELDNWGQGTLVTVSSASTKGPSVFPLAPSSKST | |
| | | SGGTAALGCLVKDYFPEPVTVSWNSGALTSGVHTFPAVLQSSG | |
| | Heavy Chain | QVQLVQSGAEVKKPGASVKVSCKASGYTFTGYYMHWVRQAPGQGL | 1568 |
| | Variable | EWMGWINPNSGAINYAQKFQGRVTMTRDTSISTAYMELSSLRSDDTA | |
| | Region | VYYCARVGRFSIFGVELDNWGQGTLVTVSS | |
| | HCDR1 | GYYMH | 1569 |
| | HCDR2 | WINPNSGAINYAQKFQG | 1570 |
| | HCDR3 | VGRFSIFGVELDN | 1571 |
| | HFRW1 | QVQLVQSGAEVKKPGASVKVSCKASGYTFT | 1572 |
| | HFRW2 | WVRQAPGQGLEWMG | 1573 |
| | HFRW3 | RVTMTRDTSISTAYMELSSLRSDDTAVYYCAR | 1574 |
| | HFRW4 | WGQGTLVTVSS | 1575 |
| | Light Chain | QSALTQPPSASGSPGQSVTISCTGTSSDVGGYNFVSWYQQHPGKAPKL | 1576 |
| | | MIYEVSKRPSGVPDRFSGSKSGNTASLTVSGLQAEDEADYYCSSYGG | |
| | | SNNLIFGGGTRLTVLGQPKAAPSVTLFPPSSEELQANKATLVCLISDFY | |
| | | PGAVTVAWKADSSPVKAGVETTTPSKQSNNKYAASSYLSLTPEQWK | |
| | | SH | |
| | Light Chain | QSALTQPPSASGSPGQSVTISCTGTSSDVGGYNFVSWYQQHPGKAPKL | 1577 |
| | Variable | MIYEVSKRPSGVPDRFSGSKSGNTASLTVSGLQAEDEADYYCSSYGG | |
| | Region | SNNLIFGGGTRLTVL | |
| | LCDR1 | TGTSSDVGGYNFVS | 1578 |
| | LCDR2 | EVSKRPS | 1579 |
| | LCDR3 | SSYGGSNNLI | 1580 |
| | LFRW1 | QSALTQPPSASGSPGQSVTISC | 1581 |
| | LFRW2 | WYQQHPGKAPKLMIY | 1582 |
| | LFRW3 | GVPDRFSGSKSGNTASLTVSGLQAEDEADYYC | 1583 |
| | LFRW4 | FGGGTRLTVL | 1584 |
| S564-275 | Heavy Chain | QVQLQESGPGLVKPSETLSLTCTVSGGSISSYYWSWIRQPPGKGLEWI | 1585 |
| (NP) | | GYIYYSGSTKYNPSLKSRVTISVDTSKKQFSLKLSSVTAADTAVYYCA | |
| | | RHIKIGVVGGLTFDFWGQGTLVTVSSGSASAPTLFPLVSCENSPSDTSS | |
| | | V | |
| | Heavy Chain | QVQLQESGPGLVKPSETLSLTCTVSGGSISSYYWSWIRQPPGKGLEWI | 1586 |
| | Variable | GYIYYSGSTKYNPSLKSRVTISVDTSKKQFSLKLSSVTAADTAVYYCA | |
| | Region | RHIKIGVVGGLTFDFWGQGTLVTVSS | |
| | HCDR1 | SYYWS | 1587 |
| | HCDR2 | YIYYSGSTKYNPSLKS | 1588 |
| | HCDR3 | HIKIGVVGGLTFDF | 1589 |
| | HFRW1 | QVQLQESGPGLVKPSETLSLTCTVSGGSIS | 1590 |
| | HFRW2 | WIRQPPGKGLEWIG | 1591 |
| | HFRW3 | RVTISVDTSKKQFSLKLSSVTAADTAVYYCAR | 1592 |
| | HFRW4 | WGQGTLVTVSS | 1593 |
| | Light Chain | DIQMTQSPSSLSASIGDRVTITCRASQSISTYLNWYQQKPGKAPKLLIY | 1594 |
| | | AASSLQSGVPSRFSGSGSGADFTLTISSLQPEDFATYYCQQSYSTPLTF | |
| | | GGGTKVEIKRTVAAPSVFIFPPSDEQLKSGTASVVCLLNNFYPREAKV | |
| | | QWKVDNA | |
| | Light Chain | DIQMTQSPSSLSASIGDRVTITCRASQSISTYLNWYQQKPGKAPKLLIY | 1595 |
| | Variable | AASSLQSGVPSRFSGSGSGADFTLTISSLQPEDFATYYCQQSYSTPLTF | |
| | Region | GGGTKVEIK | |
| | LCDR1 | RASQSISTYLN | 1596 |
| | LCDR2 | AASSLQS | 1597 |
| | LCDR3 | QQSYSTPLT | 1598 |
| | LFRW1 | DIQMTQSPSSLSASIGDRVTITC | 1599 |
| | LFRW2 | WYQQKPGKAPKLLIY | 1600 |
| | LFRW3 | GVPSRFSGSGSGADFTLTISSLQPEDFATYYC | 1601 |
| | LFRW4 | FGGGTKVEIK | 1602 |
| S564-287 | Heavy Chain | QVQLVQSGAEVKKPGASVKVSCKASGYTFTGYYMHWVRQAPGQGL | 1603 |
| (ORF8) | | EWMGWINPNSGGTNYAQKFQGRVTMTRDTSISTAYMELSRLRCDDT | |
| | | AVYYCARASTPYSSGSWADYWGQGTLVTVSSGSASAPTLFPLVSCEN | |
| | | SPSDTSSV | |
| | Heavy Chain | QVQLVQSGAEVKKPGASVKVSCKASGYTFTGYYMHWVRQAPGQGL | 1604 |
| | Variable | EWMGWINPNSGGTNYAQKFQGRVTMTRDTSISTAYMELSRLRCDDT | |
| | Region | AVYYCARASTPYSSGSWADYWGQGTLVTVSS | |
| | HCDR1 | GYYMH | 1605 |
| | HCDR2 | WINPNSGGTNYAQKFQG | 1606 |
| | HCDR3 | ASTPYSSGSWADY | 1607 |
| | HFRW1 | QVQLVQSGAEVKKPGASVKVSCKASGYTFT | 1608 |
| | HFRW2 | WVRQAPGQGLEWMG | 1609 |
| | HFRW3 | RVTMTRDTSISTAYMELSRLRCDDTAVYYCAR | 1610 |
| | HFRW4 | WGQGTLVTVSS | 1611 |
| | Light Chain | QSALTQPASVSGSPGQSITISCTGTSSDVGGYNYVSWYQQHPGKAPKL | 1612 |
| | | MIYDVSNRPSGVSNRFSGSKSGNTASLTISGLQAEDEADYYCSSYASS | |
| | | STWVFGGGTKLTVLGQPKAAPSVTLFPPSSEELQANKATLVCLISDFY | |
| | | PGAVTVAWKADSSPVKAGVETTTPSKQSNNKYAASSYLSLT | |
| | Light Chain | QSALTQPASVSGSPGQSITISCTGTSSDVGGYNYVSWYQQHPGKAPKL | 1613 |
| | Variable | MIYDVSNRPSGVSNRFSGSKSGNTASLTISGLQAEDEADYYCSSYASS | |
| | Region | STWVFGGGTKLTVL | |
| | LCDR1 | TGTSSDVGGYNYVS | 1614 |
| | LCDR2 | DVSNRPS | 1615 |
| | LCDR3 | SSYASSSTWV | 1616 |
| | LFRW1 | QSALTQPASVSGSPGQSITISC | 1617 |
| | LFRW2 | WYQQHPGKAPKLMIY | 1618 |
| | LFRW3 | GVSNRFSGSKSGNTASLTISGLQAEDEADYYC | 1619 |
| | LFRW4 | FGGGTKLTVL | 1620 |
| S116-2822 | Heavy Chain | QVQLVESGGGVVQPGRSLRLSCAASGFTFSSYGMHWVRQAPGKGLE | 1825 |
| (Spike) | | WVAVISYDGSNKYYADSVKGRFTISRDNSKNTLYLQMNSLRAEDTA | |
| | | VYYCAKGDYYGSGSQYYFDYWGQGTLVTVSSGSASAPTLFPLVSCE | |
| | | NSPSDTSSV | |
| | Heavy Chain | QVQLVESGGGVVQPGRSLRLSCAASGFTFSSYGMHWVRQAPGKGLE | 1826 |
| | Variable | WVAVISYDGSNKYYADSVKGRFTISRDNSKNTLYLQMNSLRAEDTA | |
| | Region | VYYCAKGDYYGSGSQYYFDYWGQGTLVTVSS | |
| | HCDR1 | SYGMH | 1827 |
| | HCDR2 | VISYDGSNKYYADSVKG | 1828 |
| | HCDR3 | GDYYGSGSQYYFDY | 1829 |
| | HFRW1 | QVQLVESGGGVVQPGRSLRLSCAASGFTFS | 1830 |
| | HFRW2 | WVRQAPGKGLEWVA | 1831 |
| | HFRW3 | RFTISRDNSKNTLYLQMNSLRAEDTAVYYCAK | 1832 |
| | HFRW4 | WGQGTLVTVSS | 1833 |
| | Light Chain | DIQMTQSPSTLSASVGDRVTITCRASQSISSWLAWYQQKPGKAPKLLI | 1834 |
| | | YDASSLESGVPSRFSGSGSGTEFTLTISSLQPDDFATYYCQQYNSYSQT | |
| | | FGQGTKLEIKRTVAAPSVFIFPPSDEQLKSGTASVVCLLNNFYPREAK | |
| | | VQWKVDN~ | |
| | Light Chain | DIQMTQSPSTLSASVGDRVTITCRASQSISSWLAWYQQKPGKAPKLLI | 1835 |
| | Variable | YDASSLESGVPSRFSGSGSGTEFTLTISSLQPDDFATYYCQQYNSYSQT | |
| | Region | FGQGTKLEIK | |
| | LCDR1 | RASQSISSWLA | 1836 |
| | LCDR2 | DASSLES | 1837 |
| | LCDR3 | QQYNSYSQT | 1838 |
| | LFRW1 | DIQMTQSPSTLSASVGDRVTITC | 1839 |
| | LFRW2 | WYQQKPGKAPKLLIY | 1840 |
| | LFRW3 | GVPSRFSGSGSGTEFTLTISSLQPDDFATYYC | 1841 |
| | LFRW4 | FGQGTKLEIK | 1842 |
| S116-2825 | Heavy Chain | EVQLVESGGGLVQPGGSLRLSCAASGFTFSSYWMHWVRQAPGKGLV | 1843 |
| (Spike) | | WVSRINSDGSSTSYADSVKGRFTISRDNAKNTLYLQMNSLRAEDTAV | |
| | | YYCARVVLTYYYDSSGYQNAFDIWGQGTMVTVSSGSASAPTLFPLVS | |
| | | CENSPSDTSSV | |
| | Heavy Chain | EVQLVESGGGLVQPGGSLRLSCAASGFTFSSYWMHWVRQAPGKGLV | 1844 |
| | Variable | WVSRINSDGSSTSYADSVKGRFTISRDNAKNTLYLQMNSLRAEDTAV | |
| | Region | YYCARVVLTYYYDSSGYQNAFDIWGQGTMVTVSS | |
| | HCDR1 | SYWMH | 1845 |
| | HCDR2 | RINSDGSSTSYADSVKG | 1846 |
| | HCDR3 | VVLTYYYDSSGYQNAFDI | 1847 |
| | HFRW1 | EVQLVESGGGLVQPGGSLRLSCAASGFTFS | 1848 |
| | HFRW2 | WVRQAPGKGLVWVS | 1849 |
| | HFRW3 | RFTISRDNAKNTLYLQMNSLRAEDTAVYYCAR | 1850 |
| | HFRW4 | WGQGTMVTVSS | 1851 |
| | Light Chain | SSELTQDPAVSVALGQTVRITCQGDSLRSYYASWYQQKPGQAPVLVI | 1852 |
| | | YGKNNRPSGIPDRFSGSSSGNTASLTITGAQAEDEADYYCNSRDSSGN | |
| | | LVVFGGGTKLTVLGQPKAAPSVTLFPPSSEELQANKATLVCLISDFYP | |
| | | GAVTVAWKADSSPVKAGVETTKPSKQSNNKYAASS~ | |
| | Light Chain | SSELTQDPAVSVALGQTVRITCQGDSLRSYYASWYQQKPGQAPVLVI | 1853 |
| | Variable | YGKNNRPSGIPDRFSGSSSGNTASLTITGAQAEDEADYYCNSRDSSGN | |
| | Region | LVVFGGGTKLTVL | |
| | LCDR1 | QGDSLRSYYAS | 1854 |
| | LCDR2 | GKNNRPS | 1855 |
| | LCDR3 | NSRDSSGNLVV | 1856 |
| | LFRW1 | SSELTQDPAVSVALGQTVRITC | 1857 |
| | LFRW2 | WYQQKPGQAPVLVIY | 1858 |
| | LFRW3 | GIPDRFSGSSSGNTASLTITGAQAEDEADYYC | 1859 |
| | LFRW4 | FGGGTKLTVL | 1860 |
| S116-3179 | Heavy Chain | QVQLQESGPGLVKPSETLSLTCTVSGGSISSYYWSWIRQPPGKGLEWI | 1861 |
| (Spike) | | GYIYYSGSTNYNPSLKSRVTISVDTSKNQFSLKLTSVTAADTAVYYCA | |
| | | RCALLLGNAFDIWGQGTMVTVSSASTKGPSVFPLAPCSR~ | |
| | Heavy Chain | QVQLQESGPGLVKPSETLSLTCTVSGGSISSYYWSWIRQPPGKGLEWI | 1862 |
| | Variable | GYIYYSGSTNYNPSLKSRVTISVDTSKNQFSLKLTSVTAADTAVYYCA | |
| | Region | RCALLLGNAFDIWGQGTMVTVSS | |
| | HCDR1 | SYYWS | 1863 |
| | HCDR2 | YIYYSGSTNYNPSLKS | 1864 |
| | HCDR3 | CALLLGNAFDI | 1865 |
| | HFRW1 | QVQLQESGPGLVKPSETLSLTCTVSGGSIS | 1866 |
| | HFRW2 | WIRQPPGKGLEWIG | 1867 |
| | HFRW3 | RVTISVDTSKNQFSLKLTSVTAADTAVYYCAR | 1868 |
| | HFRW4 | WGQGTMVTVSS | 1869 |
| | Light Chain | DIQMTQSPSSVSASVGDRVTITCRASQGISSWLAWYQQKPGKAPKLLI | 1870 |
| | | YAAFSLQSGVPSRFSGSGSGTDFTLTISSLQPEDFATYYCQQANSFPRG | |
| | | LSFGGGTKVEIKRTVAAPSVFIFPPSDEQLKSGTASVVCLLNNFYPREA | |
| | | KVQWKVDN~ | |
| | Light Chain | DIQMTQSPSSVSASVGDRVTITCRASQGISSWLAWYQQKPGKAPKLLI | 1871 |
| | Variable | YAAFSLQSGVPSRFSGSGSGTDFTLTISSLQPEDFATYYCQQANSFPRG | |
| | Region | LSFGGGTKVEIK | |
| | LCDR1 | RASQGISSWLA | 1872 |
| | LCDR2 | AAFSLQS | 1873 |
| | LCDR3 | QQANSFPRGLS | 1874 |
| | LFRW1 | DIQMTQSPSSVSASVGDRVTITC | 1875 |
| | LFRW2 | WYQQKPGKAPKLLIY | 1876 |
| | LFRW3 | GVPSRFSGSGSGTDFTLTISSLQPEDFATYYC | 1877 |
| | LFRW4 | FGGGTKVEIK | 1878 |
| S144-121 | Heavy Chain | EVHLLESGGGLVQPGGSLRLSCAASGFTFSSYAMSWVRQTPGKGLE | 1879 |
| (Spike/ | | WISAITASGSDTFHADSVKGRFTISRDNSKDTLYLQMNSLRVEDTAIY | |
| RBD) | | YCAKGSSTARPYYFDYWGQGTLVTVSSGSASAPTLFPLVSCENSPSDT | |
| | | SSV | |
| | Heavy Chain | EVHLLESGGGLVQPGGSLRLSCAASGFTFSSYAMSWVRQTPGKGLE | 1880 |
| | Variable | WISAITASGSDTFHADSVKGRFTISRDNSKDTLYLQMNSLRVEDTAIY | |
| | Region | YCAKGSSTARPYYFDYWGQGTLVTVSS | |
| | HCDR1 | SYAMS | 1881 |
| | HCDR2 | AITASGSDTFHADSVKG | 1882 |
| | HCDR3 | GSSTARPYYFDY | 1883 |
| | HFRW1 | EVHLLESGGGLVQPGGSLRLSCAASGFTFS | 1884 |
| | HFRW2 | WVRQTPGKGLEWIS | 1885 |
| | HFRW3 | RFTISRDNSKDTLYLQMNSLRVEDTAIYYCAK | 1886 |
| | HFRW4 | WGQGTLVTVSS | 1887 |
| | Light Chain | EIVLTQSPGTLSLSPGERATLSCRASQSVSSSHLAWYQQKPGQSPRLLI | 1888 |
| | | YGTSNRATGIPDRFSGSGSGTDFTLSISRLEPEDFAVYYCQEYGSSRMF | |
| | | GQGTKVEIKRTVAAPSVFIFPPSDEQLKSGTASVVCLLNNFYPREAKV | |
| | | QWKVDNALQSGNSQESVTEQDSKDSTYSLSSTLTLSKADYE | |
| | Light Chain | EIVLTQSPGTLSLSPGERATLSCRASQSVSSSHLAWYQQKPGQSPRLLI | 1889 |
| | Variable | YGTSNRATGIPDRFSGSGSGTDFTLSISRLEPEDFAVYYCQEYGSSRMF | |
| | Region | GQGTKVEIK | |
| | LCDR1 | RASQSVSSSHLA | 1890 |
| | LCDR2 | GTSNRAT | 1891 |
| | LCDR3 | QEYGSSRM | 1892 |
| | LFRW1 | EIVLTQSPGTLSLSPGERATLSC | 1893 |
| | LFRW2 | WYQQKPGQSPRLLIY | 1894 |
| | LFRW3 | GIPDRFSGSGSGTDFTLSISRLEPEDFAVYYC | 1895 |
| | LFRW4 | FGQGTKVEIK | 1896 |
| S144-1364 | Heavy Chain | EVQLVQSGAEMKKPGESLKISCKASGYYFPSYWIAWVRQMPGRGLE | 1897 |
| (Spike) | | WMGIIYPVDSETTYSPSFQGHVTISADKSISTAYLQWSSLKASDTAMY | |
| | | YCARPNYYGSGSPPGYWGQGTLVTVSSGSASAPTLFPLVSCENSPSDT | |
| | | SSVAVG~ | |
| | Heavy Chain | EVQLVQSGAEMKKPGESLKISCKASGYYFPSYWIAWVRQMPGRGLE | 1898 |
| | Variable | WMGIIYPVDSETTYSPSFQGHVTISADKSISTAYLQWSSLKASDTAMY | |
| | Region | YCARPNYYGSGSPPGYWGQGTLVTVSS | |
| | HCDR1 | SYWIA | 1899 |
| | HCDR2 | IIYPVDSETTYSPSFQG | 1900 |
| | HCDR3 | PNYYGSGSPPGY | 1901 |
| | HFRW1 | EVQLVQSGAEMKKPGESLKISCKASGYYFP | 1902 |
| | HFRW2 | WVRQMPGRGLEWMG | 1903 |
| | HFRW3 | HVTISADKSISTAYLQWSSLKASDTAMYYCAR | 1904 |
| | HFRW4 | WGQGTLVTVSS | 1905 |
| | Light Chain | EIVLTQSPGTLSLSPGERATLSCRASQGVSSNYLAWYQQKPGQAPRLL | 1906 |
| | | IYGASSRATGIPDRFSGSGSGTDFTLTISRLEPEDFAVYYCQQYGTTPN | |
| | | TFGGGTKVEIKRTVAAPSVFIFPPSDEQLKSGTASVVCLLNNFYPREA | |
| | | KVQWKVDNALQSGNSQESVTEQDSKDSTYSLSSTLTLSKADYE | |
| | Light Chain | EIVLTQSPGTLSLSPGERATLSCRASQGVSSNYLAWYQQKPGQAPRLL | 1907 |
| | Variable | IYGASSRATGIPDRFSGSGSGTDFTLTISRLEPEDFAVYYCQQYGTTPN | |
| | Region | TFGGGTKVEIK | |
| | LCDR1 | RASQGVSSNYLA | 1908 |
| | LCDR2 | GASSRAT | 1909 |
| | LCDR3 | QQYGTTPNT | 1910 |
| | LFRW1 | EIVLTQSPGTLSLSPGERATLSC | 1911 |
| | LFRW2 | WYQQKPGQAPRLLIY | 1912 |
| | LFRW3 | GIPDRFSGSGSGTDFTLTISRLEPEDFAVYYC | 1913 |
| | LFRW4 | FGGGTKVEIK | 1914 |
| S144-292 | Heavy Chain | EVQLVQSGAEVKKPGESLKISCKGSGYTFTNYWIGWVRQMPGKGLE | 1915 |
| (Spike) | | WMGIIYPGDSDTRYSPSFQGQVTISADKSISTAYLQWSSLKASDTAMY | |
| | | YCARLFCGGDCPFDYWGQGTLVTVSSASTKGPSVFPLAPCSRSTSGG | |
| | | TAALGCLVKDYFPEPVTVSWNSGALTSGVHTFPAVLQSSG | |
| | Heavy Chain | EVQLVQSGAEVKKPGESLKISCKGSGYTFTNYWIGWVRQMPGKGLE | 1916 |
| | Variable | WMGIIYPGDSDTRYSPSFQGQVTISADKSISTAYLQWSSLKASDTAMY | |
| | Region | YCARLFCGGDCPFDYWGQGTLVTVSS | |
| | HCDR1 | NYWIG | 1917 |
| | HCDR2 | IIYPGDSDTRYSPSFQG | 1918 |
| | HCDR3 | LFCGGDCPFDY | 1919 |
| | HFRW1 | EVQLVQSGAEVKKPGESLKISCKGSGYTFT | 1920 |
| | HFRW2 | WVRQMPGKGLEWMG | 1921 |
| | HFRW3 | QVTISADKSISTAYLQWSSLKASDTAMYYCAR | 1922 |
| | HFRW4 | WGQGTLVTVSS | 1923 |
| | Light Chain | DIQMTQSPSTLSASVGDRVTITCRASQSISSWLAWYQQKPGKAPNLLI | 1924 |
| | | YDASSLESGVPSRFSGSGSGTEFTLTISSLQPDDFATYYCQQYNTYPRT | |
| | | FGQGTKVEIKRTVAAPSVFIFPPSDEQLKSGTASVVCLLNNFYPREAK | |
| | | VQWKVDNALQSGNSQESVTEQDSKDSTYSLSSTLTLSKADYE~ | |
| | Light Chain | DIQMTQSPSTLSASVGDRVTITCRASQSISSWLAWYQQKPGKAPNLLI | 1925 |
| | Variable | YDASSLESGVPSRFSGSGSGTEFTLTISSLQPDDFATYYCQQYNTYPRT | |
| | Region | FGQGTKVEIK | |
| | LCDR1 | RASQSISSWLA | 1926 |
| | LCDR2 | DASSLES | 1927 |
| | LCDR3 | QQYNTYPRT | 1928 |
| | LFRW1 | DIQMTQSPSTLSASVGDRVTITC | 1929 |
| | LFRW2 | WYQQKPGKAPNLLIY | 1930 |
| | LFRW3 | GVPSRFSGSGSGTEFTLTISSLQPDDFATYYC | 1931 |
| | LFRW4 | FGQGTKVEIK | 1932 |
| S155-37 | Heavy Chain | EVQLLESGGGLVQPGGSLRLSCAASGFSFSNYAMSWVRQAPGKGLE | 1933 |
| (Spike/ | | WVSAVSGNGVGTFHADSVKGRFTISRDNSKDTFYLQMSGLTVDDTA | |
| RBD) | | LYYCVKGSAAARPYYFDYWGQGILVAVSSGSASAPTLFPLVSCENSP | |
| | | SDTSSV | |
| | Heavy Chain | EVQLLESGGGLVQPGGSLRLSCAASGFSFSNYAMSWVRQAPGKGLE | 1934 |
| | Variable | WVSAVSGNGVGTFHADSVKGRFTISRDNSKDTFYLQMSGLTVDDTA | |
| | Region | LYYCVKGSAAARPYYFDYWGQGILVAVSS | |
| | HCDR1 | NYAMS | 1935 |
| | HCDR2 | AVSGNGVGTFHADSVKG | 1936 |
| | HCDR3 | GSAAARPYYFDY | 1937 |
| | HFRW1 | EVQLLESGGGLVQPGGSLRLSCAASGFSFS | 1938 |
| | HFRW2 | WVRQAPGKGLEWVS | 1939 |
| | HFRW3 | RFTISRDNSKDTFYLQMSGLTVDDTALYYCVK | 1940 |
| | HFRW4 | WGQGILVAVSS | 1941 |
| | Light Chain | EIVLTQSPGTLSLSPGERATLSCRASQTVSSNYLAWYQQKPAQGPRLV | 1942 |
| | | IYGASNRATGIPDRFSGSGSGTDFTLTISRLEPEDFAVYYCQQYGNSRI | |
| | | FGQGTKVEIKRTVAAPSVFIFPPSDEQLKSGTASVVCLLNNFYPREAK | |
| | | VQWKVDN~ | |
| | Light Chain | EIVLTQSPGTLSLSPGERATLSCRASQTVSSNYLAWYQQKPAQGPRLV | 1943 |
| | Variable | IYGASNRATGIPDRFSGSGSGTDFTLTISRLEPEDFAVYYCQQYGNSRI | |
| | Region | FGQGTKVEIK | |
| | LCDR1 | RASQTVSSNYLA | 1944 |
| | LCDR2 | GASNRAT | 1945 |
| | LCDR3 | QQYGNSRI | 1946 |
| | LFRW1 | EIVLTQSPGTLSLSPGERATLSC | 1947 |
| | LFRW2 | WYQQKPAQGPRLVIY | 1948 |
| | LFRW3 | GIPDRFSGSGSGTDFTLTISRLEPEDFAVYYC | 1949 |
| | LFRW4 | FGQGTKVEIK | 1950 |
| S166-1318 | Heavy Chain | EVQLVESGGGLVQPGGSLRLSCAASGFTFTIYWMSWVRQAPGKGLE | 1951 |
| (Spike) | | WVANIKQDGSEKYYVDSVKGRFTISRDNAKNSLYLQMNSLRAEDTA | |
| | | VYYCARDGIAVAGGFDYWGQGTLVTVSSGSASAPTLFPLVSCENSPS | |
| | | DTSSV | |
| | Heavy Chain | EVQLVESGGGLVQPGGSLRLSCAASGFTFTIYWMSWVRQAPGKGLE | 1952 |
| | Variable | WVANIKQDGSEKYYVDSVKGRFTISRDNAKNSLYLQMNSLRAEDTA | |
| | Region | VYYCARDGIAVAGGFDYWGQGTLVTVSS | |
| | HCDR1 | IYWMS | 1953 |
| | HCDR2 | NIKQDGSEKYYVDSVKG | 1954 |
| | HCDR3 | DGIAVAGGFDY | 1955 |
| | HFRW1 | EVQLVESGGGLVQPGGSLRLSCAASGFTFT | 1956 |
| | HFRW2 | WVRQAPGKGLEWVA | 1957 |
| | HFRW3 | RFTISRDNAKNSLYLQMNSLRAEDTAVYYCAR | 1958 |
| | HFRW4 | WGQGTLVTVSS | 1959 |
| | Light Chain | SYELTQPPSVSVSPGQTASITCSGDKLGDKYACWYQQKPGQSPVLVIY | 1960 |
| | | QDSKRPSGIPERFSGSNSGNTATLTISGTQAMDEADYYCQAWDSSTV | |
| | | VFGGGTKLTVLGQPKAAPSVTLFPPSSEELQANKATLVCLISDFYPGA | |
| | | VTVAWKADSSPVKAGVETTTPSKQSNNKYAASS~ | |
| | Light Chain | SYELTQPPSVSVSPGQTASITCSGDKLGDKYACWYQQKPGQSPVLVIY | 1961 |
| | Variable | QDSKRPSGIPERFSGSNSGNTATLTISGTQAMDEADYYCQAWDSSTV | |
| | Region | VFGGGTKLTVL | |
| | LCDR1 | SGDKLGDKYAC | 1962 |
| | LCDR2 | QDSKRPS | 1963 |
| | LCDR3 | QAWDSSTVV | 1964 |
| | LFRW1 | SYELTQPPSVSVSPGQTASITC | 1965 |
| | LFRW2 | WYQQKPGQSPVLVIY | 1966 |
| | LFRW3 | GIPERFSGSNSGNTATLTISGTQAMDEADYYC | 1967 |
| | LFRW4 | FGGGTKLTVL | 1968 |
| S166-1366 | Heavy Chain | QITLKESGPTLVKPTQTLTLTCTFSGFSLSTSGVGVGWIRQPPGKALE | 1969 |
| (Spike) | | WLALIYWDDDKRYRPSLKSRLSITKDTSKNQVVLTMTNMDPVDTAT | |
| | | YYCAHHHPILDFDYWGQGTLVTVSSGSASAPTLFPLVSCENSPSDTSS | |
| | | V | |
| | Heavy Chain | QITLKESGPTLVKPTQTLTLTCTFSGFSLSTSGVGVGWIRQPPGKALE | 1970 |
| | Variable | WLALIYWDDDKRYRPSLKSRLSITKDTSKNQVVLTMTNMDPVDTAT | |
| | Region | YYCAHHHPILDFDYWGQGTLVTVSS | |
| | HCDR1 | TSGVGVG | 1971 |
| | HCDR2 | LIYWDDDKRYRPSLKS | 1972 |
| | HCDR3 | HHPILDFDY | 1973 |
| | HFRW1 | QITLKESGPTLVKPTQTLTLTCTFSGFSLS | 1974 |
| | HFRW2 | WIRQPPGKALEWLA | 1975 |
| | HFRW3 | RLSITKDTSKNQVVLTMTNMDPVDTATYYCAH | 1976 |
| | HFRW4 | WGQGTLVTVSS | 1977 |
| | Light Chain | SYELTQPPSVSVSPGQTASITCSGDKLGDKYACWYQQKPGQSPVLVIY | 1978 |
| | | QDSKRPSGIPERFSGSNSGNTATLTISGTQAMDEADYYCQAWDSSTR | |
| | | DYVFGTGTKVTVLGQPKANPTVTLFPPSSEELQANKATLVCLISDFYP | |
| | | GAVTVAWKADGSPVKAGVETTKPSKQSNNKYAASS~ | |
| | Light Chain | SYELTQPPSVSVSPGQTASITCSGDKLGDKYACWYQQKPGQSPVLVIY | 1979 |
| | Variable | QDSKRPSGIPERFSGSNSGNTATLTISGTQAMDEADYYCQAWDSSTR | |
| | Region | DYVFGTGTKVTVL | |
| | LCDR1 | SGDKLGDKYAC | 1980 |
| | LCDR2 | QDSKRPS | 1981 |
| | LCDR3 | QAWDSSTRDYV | 1982 |
| | LFRW1 | SYELTQPPSVSVSPGQTASITC | 1983 |
| | LFRW2 | WYQQKPGQSPVLVIY | 1984 |
| | LFRW3 | GIPERFSGSNSGNTATLTISGTQAMDEADYYC | 1985 |
| | LFRW4 | FGTGTKVTVL | 1986 |
| S166-2395 | Heavy Chain | QVQLQESGPGLVKPSETLSLTCTVSGGSISTYYWSWIRQPAGKGLEWI | 1987 |
| (Spike) | | GRIYTSGSTNYNPSLKSRVTMSVDTSKNQFSLKLSSVTAADTAVYYC | |
| | | AREVTMIVLGYNWFDPWGQGTLVTVSSAPTKAPDVFPIISGCRHPKD | |
| | | NSPVVLACLITGYH | |
| | Heavy Chain | QVQLQESGPGLVKPSETLSLTCTVSGGSISTYYWSWIRQPAGKGLEWI | 1988 |
| | Variable | GRIYTSGSTNYNPSLKSRVTMSVDTSKNQFSLKLSSVTAADTAVYYC | |
| | Region | AREVTMIVLGYNWFDPWGQGTLVTVSS | |
| | HCDR1 | TYYWS | 1989 |
| | HCDR2 | RIYTSGSTNYNPSLKS | 1990 |
| | HCDR3 | EVTMIVLGYNWFDP | 1991 |
| | HFRW1 | QVQLQESGPGLVKPSETLSLTCTVSGGSIS | 1992 |
| | HFRW2 | WIRQPAGKGLEWIG | 1993 |
| | HFRW3 | RVTMSVDTSKNQFSLKLSSVTAADTAVYYCAR | 1994 |
| | HFRW4 | WGQGTLVTVSS | 1995 |
| | Light Chain | SYVLTQTPSVSVAPGQTARITCGGNNIGSKSVHWYQQKPGQAPVLVV | 1996 |
| | | HDESDRPSGIPERFFGSNSGNTATLTISRVEAGDEADYYCQVWDSSSD | |
| | | HLHVFGTGTKVTVLGQPKANPTVTLFPPSSEELQANKATLVCLISDFY | |
| | | PGAVTVAWKADGSPVKAGVETTKPSKQSNNKYAASS | |
| | Light Chain | SYVLTQTPSVSVAPGQTARITCGGNNIGSKSVHWYQQKPGQAPVLVV | 1997 |
| | Variable | HDESDRPSGIPERFFGSNSGNTATLTISRVEAGDEADYYCQVWDSSSD | |
| | Region | HLHVFGTGTKVTVL | |
| | LCDR1 | GGNNIGSKSVH | 1998 |
| | LCDR2 | DESDRPS | 1999 |
| | LCDR3 | QVWDSSSDHLHV | 2000 |
| | LFRW1 | SYVLTQTPSVSVAPGQTARITC | 2001 |
| | LFRW2 | WYQQKPGQAPVLVVH | 2002 |
| | LFRW3 | GIPERFFGSNSGNTATLTISRVEAGDEADYYC | 2003 |
| | LFRW4 | FGTGTKVTVL | 2004 |
| S166-2620 | Heavy Chain | EVQLVESGGGLVQPGGSLRLSCAASGFTFSSYWMSWVRQAPGKGLE | 2005 |
| (Spike) | | WVANIKQDGSEKYYVASVKGRFTISRDNAKNSLYLQMNSLRAEDTA | |
| | | VYYCARDSIAVAGGLDYWGQGTLVTVSSGSASAPTLFPLVSCENSPS | |
| | | DTSSV | |
| | Heavy Chain | EVQLVESGGGLVQPGGSLRLSCAASGFTFSSYWMSWVRQAPGKGLE | 2006 |
| | Variable | WVANIKQDGSEKYYVASVKGRFTISRDNAKNSLYLQMNSLRAEDTA | |
| | Region | VYYCARDSIAVAGGLDYWGQGTLVTVSS | |
| | HCDR1 | SYWMS | 2007 |
| | HCDR2 | NIKQDGSEKYYVASVKG | 2008 |
| | HCDR3 | DSIAVAGGLDY | 2009 |
| | HFRW1 | EVQLVESGGGLVQPGGSLRLSCAASGFTFS | 2010 |
| | HFRW2 | WVRQAPGKGLEWVA | 2011 |
| | HFRW3 | RFTISRDNAKNSLYLQMNSLRAEDTAVYYCAR | 2012 |
| | HFRW4 | WGQGTLVTVSS | 2013 |
| | Light Chain | SYELTQPPSVSVSPGQTASITCSGDKLGDKYACWYQQKPGQSPVLVIY | 2014 |
| | | QDSKRPSGIPERFSGSNSGNTATLTISGTQAMDEADYFCQAWDSSTVV | |
| | | FGGGTKLTVLRQPKAAPSVTLFPPSSEELQANKATLVCLISDFYPGAV | |
| | | TVAWKADSSPVKAGVETTTPSKQSNNKYAASS | |
| | Light Chain | SYELTQPPSVSVSPGQTASITCSGDKLGDKYACWYQQKPGQSPVLVIY | 2015 |
| | Variable | QDSKRPSGIPERFSGSNSGNTATLTISGTQAMDEADYFCQAWDSSTVV | |
| | Region | FGGGTKLTVL | |
| | LCDR1 | SGDKLGDKYAC | 2016 |
| | LCDR2 | QDSKRPS | 2017 |
| | LCDR3 | QAWDSSTVV | 2018 |
| | LFRW1 | SYELTQPPSVSVSPGQTASITC | 2019 |
| | LFRW2 | WYQQKPGQSPVLVIY | 2020 |
| | LFRW3 | GIPERFSGSNSGNTATLTISGTQAMDEADYFC | 2021 |
| | LFRW4 | FGGGTKLTVL | 2022 |
| S166-32 | Heavy Chain | QVQLVESGGGLVKPGGSLRLSCAASGFTFSDYYMSWIRQAPGKGLE | 2023 |
| (Spike) | | WVSYISISDTTIYYADAVQGRFTMSRDNAKNSLYLQMNSLKAEDTAV | |
| | | YYCARASPYCGGDCSFGNAFDIWGLGTMVTVSSASTKGPSVFPLAPS | |
| | | SKSTSGGTAALGCLVKDYFPEPVTVSWNSGALTSGVHTFPAVLQSSG | |
| | Heavy Chain | QVQLVESGGGLVKPGGSLRLSCAASGFTFSDYYMSWIRQAPGKGLE | 2024 |
| | Variable | WVSYISISDTTIYYADAVQGRFTMSRDNAKNSLYLQMNSLKAEDTAV | |
| | Region | YYCARASPYCGGDCSFGNAFDIWGLGTMVTVSS | |
| | HCDR1 | DYYMS | 2025 |
| | HCDR2 | YISISDTTIYYADAVQG | 2026 |
| | HCDR3 | ASPYCGGDCSFGNAFDI | 2027 |
| | HFRW1 | QVQLVESGGGLVKPGGSLRLSCAASGFTFS | 2028 |
| | HFRW2 | WIRQAPGKGLEWVS | 2029 |
| | HFRW3 | RFTMSRDNAKNSLYLQMNSLKAEDTAVYYCAR | 2030 |
| | HFRW4 | WGLGTMVTVSS | 2031 |
| | Light Chain | DIQMTQSPSTLSASVGDRVTITCRASQSIFSWLAWYQQKPGKAPKLLI | 2032 |
| | | YDASSLESGVPSRFSGSGSGTEFTLTISSLQPDDFATYYCQQYNSYWT | |
| | | FGQGTKVEIKRTVAAPSVFIFPPSDEQLKSGTASVVCLLNNFYPREAK | |
| | | VQWKVDNALQSGNSQESVTEQDSKDSTYSLSSTLTLSKADYE | |
| | Light Chain | DIQMTQSPSTLSASVGDRVTITCRASQSIFSWLAWYQQKPGKAPKLLI | 2033 |
| | Variable | YDASSLESGVPSRFSGSGSGTEFTLTISSLQPDDFATYYCQQYNSYWT | |
| | Region | FGQGTKVEIK | |
| | LCDR1 | RASQSIFSWLA | 2034 |
| | LCDR2 | DASSLES | 2035 |
| | LCDR3 | QQYNSYWT | 2036 |
| | LFRW1 | DIQMTQSPSTLSASVGDRVTITC | 2037 |
| | LFRW2 | WYQQKPGKAPKLLIY | 2038 |
| | LFRW3 | GVPSRFSGSGSGTEFTLTISSLQPDDFATYYC | 2039 |
| | LFRW4 | FGQGTKVEIK | 2040 |
| S171-1150 | Heavy Chain | EVQLVESGGGLVQPGGSLRLSCAASGFTFSSYWMSWVRQAPGKGLE | 2041 |
| (Spike) | | WVANIKQDGSEKYYVDSVKGRFTISRDNAKNSLYLQMNSLRAEDTA | |
| | | VYYCARDGIAVAGGLDYWGQGTLVTVSSAPTKAPDVFPIISGCRHPK | |
| | | DNSPVVLACLITGYH~ | |
| | Heavy Chain | EVQLVESGGGLVQPGGSLRLSCAASGFTFSSYWMSWVRQAPGKGLE | 2042 |
| | Variable | WVANIKQDGSEKYYVDSVKGRFTISRDNAKNSLYLQMNSLRAEDTA | |
| | Region | VYYCARDGIAVAGGLDYWGQGTLVTVSS | |
| | HCDR1 | SYWMS | 2043 |
| | HCDR2 | NIKQDGSEKYYVDSVKG | 2044 |
| | HCDR3 | DGIAVAGGLDY | 2045 |
| | HFRW1 | EVQLVESGGGLVQPGGSLRLSCAASGFTFS | 2046 |
| | HFRW2 | WVRQAPGKGLEWVA | 2047 |
| | HFRW3 | RFTISRDNAKNSLYLQMNSLRAEDTAVYYCAR | 2048 |
| | HFRW4 | WGQGTLVTVSS | 2049 |
| | Light Chain | SYELTQPPSVSVSPGQTASITCSGDKLGDKYACWYQQKPGQSPVLVIY | 2050 |
| | | QDSKRPSGIPERFSGSNSGNTATLTISGTQAMDEADYYCQAWDSSTV | |
| | | VFGGGTKLTVLGQPKAAPSVTLFPPSSEELQANKATLVCLISDFYPGA | |
| | | VTVAWKADSSPVKAGVETTTPSKQSNNKYAASS~ | |
| | Light Chain | SYELTQPPSVSVSPGQTASITCSGDKLGDKYACWYQQKPGQSPVLVIY | 2051 |
| | Variable | QDSKRPSGIPERFSGSNSGNTATLTISGTQAMDEADYYCQAWDSSTV | |
| | Region | VFGGGTKLTVL | |
| | LCDR1 | SGDKLGDKYAC | 2052 |
| | LCDR2 | QDSKRPS | 2053 |
| | LCDR3 | QAWDSSTVV | 2054 |
| | LFRW1 | SYELTQPPSVSVSPGQTASITC | 2055 |
| | LFRW2 | WYQQKPGQSPVLVIY | 2056 |
| | LFRW3 | GIPERFSGSNSGNTATLTISGTQAMDEADYYC | 2057 |
| | LFRW4 | FGGGTKLTVL | 2058 |
| S171-1285 | Heavy Chain | QVQLVESGGGVVQPGRSLRLSCAASGFIFSNNALHWVRQAPGKGLE | 2059 |
| (Spike) | | WVAIISYDGSNKNYAASVKGRFTISRDNSQNTVFLQMNSLRAEDTAV | |
| | | YYCARDHIAGAAKYFDYWGQGTLVTVSSASTKGPSVFPLAPSSKSTS | |
| | | GGTAALGCLVKDYFPEPVTVSWNSGALTSGVHTFPAVLQSSG | |
| | Heavy Chain | QVQLVESGGGVVQPGRSLRLSCAASGFIFSNNALHWVRQAPGKGLE | 2060 |
| | Variable | WVAIISYDGSNKNYAASVKGRFTISRDNSQNTVFLQMNSLRAEDTAV | |
| | Region | YYCARDHIAGAAKYFDYWGQGTLVTVSS | |
| | HCDR1 | NNALH | 2061 |
| | HCDR2 | IISYDGSNKNYAASVKG | 2062 |
| | HCDR3 | DHIAGAAKYFDY | 2063 |
| | HFRW1 | QVQLVESGGGVVQPGRSLRLSCAASGFIFS | 2064 |
| | HFRW2 | WVRQAPGKGLEWVA | 2065 |
| | HFRW3 | RFTISRDNSQNTVFLQMNSLRAEDTAVYYCAR | 2066 |
| | HFRW4 | WGQGTLVTVSS | 2067 |
| | Light Chain | SYELTQPPSVSVSPGQTARITCSGDALPKKFVHWYQQKSGQAPVLVIY | 2068 |
| | | EDSKRPSGIPERFSGSSSGTTATLTISGAQVEDEGDYYCYSTDSSGRGV | |
| | | FGGGTKLTVLGQPKAAPSVTLFPPSSEELQANKATLVCLISDFYPGAV | |
| | | TVAWKADSSPVKAGVETTTPSKQSNNKYAASS | |
| | Light Chain | SYELTQPPSVSVSPGQTARITCSGDALPKKFVHWYQQKSGQAPVLVIY | 2069 |
| | Variable | EDSKRPSGIPERFSGSSSGTTATLTISGAQVEDEGDYYCYSTDSSGRGV | |
| | Region | FGGGTKLTVL | |
| | LCDR1 | SGDALPKKFVH | 2070 |
| | LCDR2 | EDSKRPS | 2071 |
| | LCDR3 | YSTDSSGRGV | 2072 |
| | LFRW1 | SYELTQPPSVSVSPGQTARITC | 2073 |
| | LFRW2 | WYQQKSGQAPVLVIY | 2074 |
| | LFRW3 | GIPERFSGSSSGTTATLTISGAQVEDEGDYYC | 2075 |
| | LFRW4 | FGGGTKLTVL | 2076 |
| S171-692 | Heavy Chain | QVQLQESGPGLVKPSQTLSLTCTVSGGSISSGSYYWSWIRQPAGKGLE | 2077 |
| (Spike) | | WIGRIYTSGSTNYNPSLKSRVTISVDTSKNQFSLKLSSVTAADTAVYY | |
| | | CARESKVTMVRGGLAYYYMDVWGKGTTVTVSSAPTKAPDVFPIISG | |
| | | CRHPKDNSPVVLACLITGYH | |
| | Heavy Chain | QVQLQESGPGLVKPSQTLSLTCTVSGGSISSGSYYWSWIRQPAGKGLE | 2078 |
| | Variable | WIGRIYTSGSTNYNPSLKSRVTISVDTSKNQFSLKLSSVTAADTAVYY | |
| | Region | CARESKVTMVRGGLAYYYMDVWGKGTTVTVSS | |
| | HCDR1 | SGSYYWS | 2079 |
| | HCDR2 | RIYTSGSTNYNPSLKS | 2080 |
| | HCDR3 | ESKVTMVRGGLAYYYMDV | 2081 |
| | HFRW1 | QVQLQESGPGLVKPSQTLSLTCTVSGGSIS | 2082 |
| | HFRW2 | WIRQPAGKGLEWIG | 2083 |
| | HFRW3 | RVTISVDTSKNQFSLKLSSVTAADTAVYYCAR | 2084 |
| | HFRW4 | WGKGTTVTVSS | 2085 |
| | Light Chain | DIQMTQSPSSLSASVGDRVTITCRASQSISSYLNWYQQKPGKAPKLLIY | 2086 |
| | | AASSLQSGVPSRFSGSGSGTDFTLTISSLQPEDFATYYCQQSYSKNTFG | |
| | | QGTKLEIKRTVAAPSVFIFPPSDEQLKSGTASVVCLLNNFYPREAKVQ | |
| | | WKVDN~ | |
| | Light Chain | DIQMTQSPSSLSASVGDRVTITCRASQSISSYLNWYQQKPGKAPKLLIY | 2087 |
| | Variable | AASSLQSGVPSRFSGSGSGTDFTLTISSLQPEDFATYYCQQSYSKNTFG | |
| | Region | QGTKLEIK | |
| | LCDR1 | RASQSISSYLN | 2088 |
| | LCDR2 | AASSLQS | 2089 |
| | LCDR3 | QQSYSKNT | 2090 |
| | LFRW1 | DIQMTQSPSSLSASVGDRVTITC | 2091 |
| | LFRW2 | WYQQKPGKAPKLLIY | 2092 |
| | LFRW3 | GVPSRFSGSGSGTDFTLTISSLQPEDFATYYC | 2093 |
| | LFRW4 | FGQGTKLEIK | 2094 |
| S179-122 | Heavy Chain | EVQLVESGGGLVQPGGSLRLSCAASGFTFSTYWMSWVRQAPGKGLE | 2095 |
| (Spike/ | | WVANIKQDGSEKYYVDSVKGRFTISRDNAKNSLYLQMNSLRAEDTA | |
| RBD) | | VYYCASKLWLRGNFDYWGQGTLVTVSSASTKGPSVFPLAPSSKSTSG | |
| | | GTAALGCLVKDYFPEPVTVSWNSGALTSGVHTFPAVLQSSG | |
| | Heavy Chain | EVQLVESGGGLVQPGGSLRLSCAASGFTFSTYWMSWVRQAPGKGLE | 2096 |
| | Variable | WVANIKQDGSEKYYVDSVKGRFTISRDNAKNSLYLQMNSLRAEDTA | |
| | Region | VYYCASKLWLRGNFDYWGQGTLVTVSS | |
| | HCDR1 | TYWMS | 2097 |
| | HCDR2 | NIKQDGSEKYYVDSVKG | 2098 |
| | HCDR3 | KLWLRGNFDY | 2099 |
| | HFRW1 | EVQLVESGGGLVQPGGSLRLSCAASGFTFS | 2100 |
| | HFRW2 | WVRQAPGKGLEWVA | 2101 |
| | HFRW3 | RFTISRDNAKNSLYLQMNSLRAEDTAVYYCAS | 2102 |
| | HFRW4 | WGQGTLVTVSS | 2103 |
| | Light Chain | NFMLTQPHSVSESPGKTVTISCTGSSGSIASNYVQWYQQRPGSAPTTVI | 2104 |
| | | YEDNQRPSGVPDRFSGSIDSSSNSASLTISGLKTEDEADYYCQSYDSSN | |
| | | LVFGGGTKLTVLGQPKAAPSVTLFPPSSEELQANKATLVCLISDFYPG | |
| | | AVTVAWKADSSPVKAGVETTTPSKQSNNKYAASSYLSLTPEQWKSH | |
| | Light Chain | NFMLTQPHSVSESPGKTVTISCTGSSGSIASNYVQWYQQRPGSAPTTVI | 2105 |
| | Variable | YEDNQRPSGVPDRFSGSIDSSSNSASLTISGLKTEDEADYYCQSYDSSN | |
| | Region | LVFGGGTKLTVL | |
| | LCDR1 | TGSSGSIASNYVQ | 2106 |
| | LCDR2 | EDNQRPS | 2107 |
| | LCDR3 | QSYDSSNLV | 2108 |
| | LFRW1 | NFMLTQPHSVSESPGKTVTISC | 2109 |
| | LFRW2 | WYQQRPGSAPTTVIY | 2110 |
| | LFRW3 | GVPDRFSGSIDSSSNSASLTISGLKTEDEADYYC | 2111 |
| | LFRW4 | FGGGTKLTVL | 2112 |
| S179-20 | Heavy Chain | QVQLVESGGGVVQPGRSLRLSCAASGFTFSGYGMHWVRQAPGKGLE | 2113 |
| (Spike/ | | WVAVIWFDGSNKYYADSVKGRFTISRDNSKNTLYLQMNSLRAEDTA | |
| RBD) | | VYYCARDARYYDTSGYLGTTEFDYWGQGTLVTVSSGSASAPTLFPLV | |
| | | SCENSPSDTSSVA | |
| | Heavy Chain | QVQLVESGGGVVQPGRSLRLSCAASGFTFSGYGMHWVRQAPGKGLE | 2114 |
| | Variable | WVAVIWFDGSNKYYADSVKGRFTISRDNSKNTLYLQMNSLRAEDTA | |
| | Region | VYYCARDARYYDTSGYLGTTEFDYWGQGTLVTVSS | |
| | HCDR1 | GYGMH | 2115 |
| | HCDR2 | VIWFDGSNKYYADSVKG | 2116 |
| | HCDR3 | DARYYDTSGYLGTTEFDY | 2117 |
| | HFRW1 | QVQLVESGGGVVQPGRSLRLSCAASGFTFS | 2118 |
| | HFRW2 | WVRQAPGKGLEWVA | 2119 |
| | HFRW3 | RFTISRDNSKNTLYLQMNSLRAEDTAVYYCAR | 2120 |
| | HFRW4 | WGQGTLVTVSS | 2121 |
| | Light Chain | EVVLTQSPATLSVSPGERATLSCRASQSVSSNLAWYQQKPGQAPRLLI | 2122 |
| | | YGASTRATGIPARFSGSGSGTEFTLTISSLQSEDFAVYYCQQYNNWPR | |
| | | TFGQGTKVEIKRTVAAPSVFIFPPSDEQLKSGTASVVCLLNNFYPREA | |
| | | KVQWKVDNALQSGNSQESVTEQDSKDSTYSLSSTLTLSKADYE~ | |
| | Light Chain | EVVLTQSPATLSVSPGERATLSCRASQSVSSNLAWYQQKPGQAPRLLI | 2123 |
| | Variable | YGASTRATGIPARFSGSGSGTEFTLTISSLQSEDFAVYYCQQYNNWPR | |
| | Region | TFGQGTKVEIK | |
| | LCDR1 | RASQSVSSNLA | 2124 |
| | LCDR2 | GASTRAT | 2125 |
| | LCDR3 | QQYNNWPRT | 2126 |
| | LFRW1 | EVVLTQSPATLSVSPGERATLSC | 2127 |
| | LFRW2 | WYQQKPGQAPRLLIY | 2128 |
| | LFRW3 | GIPARFSGSGSGTEFTLTISSLQSEDFAVYYC | 2129 |
| | LFRW4 | FGQGTKVEIK | 2130 |
| S179-27 | Heavy Chain | QVQLVESGGGVVQPGRSLRLSCAASGFTFRSYGMHWVRQAPGKGLE | 2131 |
| (Spike/ | | WVAVISYDGSNKNYADSVKGRLTISRDNSKNTLYLQMNSLRAEDTA | |
| RBD) | | VYYCAKDRGGYSSGWTYYYYGMDVWGQGTTVTVSSASTKGPSVFP | |
| | | LAPSSKSTSGGTAALGCLVKDYFPEPVTVSWNSGALTSGVHTFPAVL | |
| | | QSSGLYSLSSVVTVPSSSLGTQTYICNVNHKPSNTKVD~ | |
| | Heavy Chain | QVQLVESGGGVVQPGRSLRLSCAASGFTFRSYGMHWVRQAPGKGLE | 2132 |
| | Variable | WVAVISYDGSNKNYADSVKGRLTISRDNSKNTLYLQMNSLRAEDTA | |
| | Region | VYYCAKDRGGYSSGWTYYYYGMDVWGQGTTVTVSS | |
| | HCDR1 | SYGMH | 2133 |
| | HCDR2 | VISYDGSNKNYADSVKG | 2134 |
| | HCDR3 | DRGGYSSGWTYYYYGMDV | 2135 |
| | HFRW1 | QVQLVESGGGVVQPGRSLRLSCAASGFTFR | 2136 |
| | HFRW2 | WVRQAPGKGLEWVA | 2137 |
| | HFRW3 | RLTISRDNSKNTLYLQMNSLRAEDTAVYYCAK | 2138 |
| | HFRW4 | WGQGTTVTVSS | 2139 |
| | Light Chain | DIQMTQSPSSLSASVGDRVTITCQASQDISNYLNWYQQKPGKAPKLLI | 2140 |
| | | YDASNLETGVPSRFSGSGSGTDFTFTISSLQPEDIATYYCQQYDNLPLT | |
| | | FGGGTKVEIKRTVAAPSVFIFPPSDEQLKSGTASVVCLLNNFYPREAK | |
| | | VQWKVDNALQSGNSQESVTEQDSKDSTYSLSSTLTLSKADYE~ | |
| | Light Chain | DIQMTQSPSSLSASVGDRVTITCQASQDISNYLNWYQQKPGKAPKLLI | 2141 |
| | Variable | YDASNLETGVPSRFSGSGSGTDFTFTISSLQPEDIATYYCQQYDNLPLT | |
| | Region | FGGGTKVEIK | |
| | LCDR1 | QASQDISNYLN | 2142 |
| | LCDR2 | DASNLET | 2143 |
| | LCDR3 | QQYDNLPLT | 2144 |
| | LFRW1 | DIQMTQSPSSLSASVGDRVTITC | 2145 |
| | LFRW2 | WYQQKPGKAPKLLIY | 2146 |
| | LFRW3 | GVPSRFSGSGSGTDFTFTISSLQPEDIATYYC | 2147 |
| | LFRW4 | FGGGTKVEIK | 2148 |
| S179-28 | Heavy Chain | EVQLLESGGGLVQPGGSLRLSCAASGFTFSSYAMSWVRQAPGKGLE | 2149 |
| (Spike/ | | WVSAIRGSGGSTYYADSVKGRFTISRDNSKNTLYLQMNSLRAEDTAV | |
| RBD) | | YYCAKGVRSSDDYFEYWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVD~ | |
| | Heavy Chain | EVQLLESGGGLVQPGGSLRLSCAASGFTFSSYAMSWVRQAPGKGLE | 2150 |
| | Variable | WVSAIRGSGGSTYYADSVKGRFTISRDNSKNTLYLQMNSLRAEDTAV | |
| | Region | YYCAKGVRSSDDYFEYWGQGTLVTVSS | |
| | HCDR1 | SYAMS | 2151 |
| | HCDR2 | AIRGSGGSTYYADSVKG | 2152 |
| | HCDR3 | GVRSSDDYFEY | 2153 |
| | HFRW1 | EVQLLESGGGLVQPGGSLRLSCAASGFTFS | 2154 |
| | HFRW2 | WVRQAPGKGLEWVS | 2155 |
| | HFRW3 | RFTISRDNSKNTLYLQMNSLRAEDTAVYYCAK | 2156 |
| | HFRW4 | WGQGTLVTVSS | 2157 |
| | Light Chain | DIQMTQSPSTLSASVGDRVTITCRASQSITSWLAWYQQKPGKAPKLLI | 2158 |
| | | YDASSLESGVPSRFSGSGSGTEFTLTISSLQPDDFATYYCQHYNSYPW | |
| | | TFGQGTKVEIKRTVAAPSVFIFPPSDEQLKSGTASVVCLLNNFYPREA | |
| | | KVQWKVDNALQSGNSQESVTEQDSKDSTYSLSSTLTLSKADYE~ | |
| | Light Chain | DIQMTQSPSTLSASVGDRVTITCRASQSITSWLAWYQQKPGKAPKLLI | 2159 |
| | Variable | YDASSLESGVPSRFSGSGSGTEFTLTISSLQPDDFATYYCQHYNSYPW | |
| | Region | TFGQGTKVEIK | |
| | LCDR1 | RASQSITSWLA | 2160 |
| | LCDR2 | DASSLES | 2161 |
| | LCDR3 | QHYNSYPWT | 2162 |
| | LFRW1 | DIQMTQSPSTLSASVGDRVTITC | 2163 |
| | LFRW2 | WYQQKPGKAPKLLIY | 2164 |
| | LFRW3 | GVPSRFSGSGSGTEFTLTISSLQPDDFATYYC | 2165 |
| | LFRW4 | FGQGTKVEIK | 2166 |
| S210-1139 | Heavy Chain | EVQLVQSGAEVKKPGESLKISCKGSGYYFPSYWIGWVRQKPGNGPE | 2167 |
| (Spike) | | WMGIIHPGDSESTYSPSFQGQVTISADKSISTAYLQWSSLKASDTAMY | |
| | | YCARPFYYGSESPPGYWGQGTLVTVSSGSASAPTLFPLVSCENSPSDT | |
| | | SSV | |
| | Heavy Chain | EVQLVQSGAEVKKPGESLKISCKGSGYYFPSYWIGWVRQKPGNGPE | 2168 |
| | Variable | WMGIIHPGDSESTYSPSFQGQVTISADKSISTAYLQWSSLKASDTAMY | |
| | Region | YCARPFYYGSESPPGYWGQGTLVTVSS | |
| | HCDR1 | SYWIG | 2169 |
| | HCDR2 | IIHPGDSESTYSPSFQG | 2170 |
| | HCDR3 | PFYYGSESPPGY | 2171 |
| | HFRW1 | EVQLVQSGAEVKKPGESLKISCKGSGYYFP | 2172 |
| | HFRW2 | WVRQKPGNGPEWMG | 2173 |
| | HFRW3 | QVTISADKSISTAYLQWSSLKASDTAMYYCAR | 2174 |
| | HFRW4 | WGQGTLVTVSS | 2175 |
| | Light Chain | EIVLTQSPGTLSLSPGERATLSCRASQSVSSSYLAWYQQKPGQAPRLLI | 2176 |
| | | YGASSRATGIPDRFSGSGSGTDFTLTISRLEAEDFAVYYCQLFGSSPTW | |
| | | TFGQGTKVEIKRTVAAPSVFIFPPSDEQLKSGTASVVCLLNNFYPREA | |
| | | KVQWKVDN~ | |
| | Light Chain | EIVLTQSPGTLSLSPGERATLSCRASQSVSSSYLAWYQQKPGQAPRLLI | 2177 |
| | Variable | YGASSRATGIPDRFSGSGSGTDFTLTISRLEAEDFAVYYCQLFGSSPTW | |
| | Region | TFGQGTKVEIK | |
| | LCDR1 | RASQSVSSSYLA | 2178 |
| | LCDR2 | GASSRAT | 2179 |
| | LCDR3 | QLFGSSPTWT | 2180 |
| | LFRW1 | EIVLTQSPGTLSLSPGERATLSC | 2181 |
| | LFRW2 | WYQQKPGQAPRLLIY | 2182 |
| | LFRW3 | GIPDRFSGSGSGTDFTLTISRLEAEDFAVYYC | 2183 |
| | LFRW4 | FGQGTKVEIK | 2184 |
| S210-1262 | Heavy Chain | QLQLQESGPGLMKPSETLSLTCTVSGGSISRSNYYWGWIRQPPGKGLE | 2185 |
| (Spike) | | WIGSIYYSGSTYYNPSLKSRVTISVDTSQNQFSLKMSSVTAADTAVYY | |
| | | CASLFDYGDNYWGQGTLVTVSSASTKGPSVFPLAPSSKS~ | |
| | Heavy Chain | QLQLQESGPGLMKPSETLSLTCTVSGGSISRSNYYWGWIRQPPGKGLE | 2186 |
| | Variable | WIGSIYYSGSTYYNPSLKSRVTISVDTSQNQFSLKMSSVTAADTAVYY | |
| | Region | CASLFDYGDNYWGQGTLVTVSS | |
| | HCDR1 | RSNYYWG | 2187 |
| | HCDR2 | SIYYSGSTYYNPSLKS | 2188 |
| | HCDR3 | LFDYGDNY | 2189 |
| | HFRW1 | QLQLQESGPGLMKPSETLSLTCTVSGGSIS | 2190 |
| | HFRW2 | WIRQPPGKGLEWIG | 2191 |
| | HFRW3 | RVTISVDTSQNQFSLKMSSVTAADTAVYYCAS | 2192 |
| | HFRW4 | WGQGTLVTVSS | 2193 |
| | Light Chain | QLVLTQSPSASASLGASVKLTCTLSSGHSSYAIAWHQQQPERGPRYL | 2194 |
| | | MKLNGDGSHSKGDGIPDRFSGSSSGAERYLTISSLQSEDEADYYCQT | |
| | | WGTDIQVFGGGTKLTVLGQPKAAPSVTLFPPSSEELQANKATLVCLIS | |
| | | DFYPGAVTVAWKADSSPVKAGVETTTPSKQSNNKYAASS~ | |
| | Light Chain | QLVLTQSPSASASLGASVKLTCTLSSGHSSYAIAWHQQQPERGPRYL | 2195 |
| | Variable | MKLNGDGSHSKGDGIPDRFSGSSSGAERYLTISSLQSEDEADYYCQT | |
| | Region | WGTDIQVFGGGTKLTVL | |
| | LCDR1 | TLSSGHSSYAIA | 2196 |
| | LCDR2 | LNGDGSHSKGD | 2197 |
| | LCDR3 | QTWGTDIQV | 2198 |
| | LFRW1 | QLVLTQSPSASASLGASVKLTC | 2199 |
| | LFRW2 | WHQQQPERGPRYLMK | 2200 |
| | LFRW3 | GIPDRFSGSSSGAERYLTISSLQSEDEADYYC | 2201 |
| | LFRW4 | FGGGTKLTVL | 2202 |
| S210-1611 | Heavy Chain | QVQLVQSGAEVKKPGSSVKVSCKASGGTFSSYAISWVRQARGQGLE | 2203 |
| (Spike) | | WMGGIIPIFGTANYPQKFQGRVTITADESTSTAYMELSSLRSEDTAVY | |
| | | YCARYHAYDSSGYYVDYWGQGTLVTVSSASPTSPKVFPLSLCSTQPD | |
| | | GNVVIACLVQGFFPQEPLSVTWSESGQGVTARNF~ | |
| | Heavy Chain | QVQLVQSGAEVKKPGSSVKVSCKASGGTFSSYAISWVRQARGQGLE | 2204 |
| | Variable | WMGGIIPIFGTANYPQKFQGRVTITADESTSTAYMELSSLRSEDTAVY | |
| | Region | YCARYHAYDSSGYYVDYWGQGTLVTVSS | |
| | HCDR1 | SYAIS | 2205 |
| | HCDR2 | GIIPIFGTANYPQKFQG | 2206 |
| | HCDR3 | YHAYDSSGYYVDY | 2207 |
| | HFRW1 | QVQLVQSGAEVKKPGSSVKVSCKASGGTFS | 2208 |
| | HFRW2 | WVRQARGQGLEWMG | 2209 |
| | HFRW3 | RVTITADESTSTAYMELSSLRSEDTAVYYCAR | 2210 |
| | HFRW4 | WGQGTLVTVSS | 2211 |
| | Light Chain | EIVLTQSPATLSLSPGERATLSCRASQSISSFLAWYQQKPGQAPRLLIY | 2212 |
| | | DASNRATGIPARFSGSGSGTDFILTINNLEPEDFAVYYCQQRSNWPPK | |
| | | LTFGGGTKVEIKRTVAAPSVFIFPPSDEQLKSGTASVVCLLNNFYPREA | |
| | | KVQWKVDN~ | |
| | Light Chain | EIVLTQSPATLSLSPGERATLSCRASQSISSFLAWYQQKPGQAPRLLIY | 2213 |
| | Variable | DASNRATGIPARFSGSGSGTDFILTINNLEPEDFAVYYCQQRSNWPPK | |
| | Region | LTFGGGTKVEIK | |
| | LCDR1 | RASQSISSFLA | 2214 |
| | LCDR2 | DASNRAT | 2215 |
| | LCDR3 | QQRSNWPPKLT | 2216 |
| | LFRW1 | EIVLTQSPATLSLSPGERATLSC | 2217 |
| | LFRW2 | WYQQKPGQAPRLLIY | 2218 |
| | LFRW3 | GIPARFSGSGSGTDFILTINNLEPEDFAVYYC | 2219 |
| | LFRW4 | FGGGTKVEIK | 2220 |
| S210-727 | Heavy Chain | QVQLQESGPGLVKPSETLSLTCTVSGGSMSSSYWSWIRQPPGKGLEWI | 2221 |
| (Spike) | | GYIYYRGSTNYNPSLKTRVTMSVDTSKNQFSMKMTFMTAADTAVYY | |
| | | CAREAAFNWFDSWGQGTLVTVSSGSASAPTLFPLVSCENSPSDTSSV | |
| | Heavy Chain | QVQLQESGPGLVKPSETLSLTCTVSGGSMSSSYWSWIRQPPGKGLEWI | 2222 |
| | Variable | GYIYYRGSTNYNPSLKTRVTMSVDTSKNQFSMKMTFMTAADTAVYY | |
| | Region | CAREAAFNWFDSWGQGTLVTVSS | |
| | HCDR1 | SSYWS | 2223 |
| | HCDR2 | YIYYRGSTNYNPSLKT | 2224 |
| | HCDR3 | EAAFNWFDS | 2225 |
| | HFRW1 | QVQLQESGPGLVKPSETLSLTCTVSGGSMS | 2226 |
| | HFRW2 | WIRQPPGKGLEWIG | 2227 |
| | HFRW3 | RVTMSVDTSKNQFSMKMTFMTAADTAVYYCAR | 2228 |
| | HFRW4 | WGQGTLVTVSS | 2229 |
| | Light Chain | DIQMTQSPSSLSASVGDRVTITCRASQGISSYLAWFQQKPGKAPKSLIY | 2230 |
| | | AASSLQSGVPSKFSGSGSGTDFTLTISSLQPEDFATYYCQQYNRYPPTF | |
| | | GGGTKVEIKRTVAAPSVFIFPPSDEQLKSGTASVVCLLNNFYPREAKV | |
| | | QWKVDN~ | |
| | Light Chain | DIQMTQSPSSLSASVGDRVTITCRASQGISSYLAWFQQKPGKAPKSLIY | 2231 |
| | Variable | AASSLQSGVPSKFSGSGSGTDFTLTISSLQPEDFATYYCQQYNRYPPTF | |
| | Region | GGGTKVEI | |
| | LCDR1 | RASQGISSYLA | 2232 |
| | LCDR2 | AASSLQS | 2233 |
| | LCDR3 | QQYNRYPPT | 2234 |
| | LFRW1 | DIQMTQSPSSLSASVGDRVTITC | 2235 |
| | LFRW2 | WFQQKPGKAPKSLIY | 2236 |
| | LFRW3 | GVPSKFSGSGSGTDFTLTISSLQPEDFATYYC | 2237 |
| | LFRW4 | FGGGTKVEI | 2238 |
| S210-852 | Heavy Chain | EVQLVESGGGLVQPGGSLRLSCAASGFTLSIYWMSWVRQAPGKGLE | 2239 |
| (Spike) | | WVANIKQDGREKYHVDSVKGRFTISRDNANNSLYLQMNNLRAEDTA | |
| | | VYFCARDGIAVAGGFDYWGQGTLVTVSSGSASAPTLFPLVSCENSPS | |
| | | DTSSV | |
| | Heavy Chain | EVQLVESGGGLVQPGGSLRLSCAASGFTLSIYWMSWVRQAPGKGLE | 2240 |
| | Variable | WVANIKQDGREKYHVDSVKGRFTISRDNANNSLYLQMNNLRAEDTA | |
| | Region | VYFCARDGIAVAGGFDYWGQGTLVTVSS | |
| | HCDR1 | IYWMS | 2241 |
| | HCDR2 | NIKQDGREKYHVDSVKG | 2242 |
| | HCDR3 | DGIAVAGGFDY | 2243 |
| | HFRW1 | EVQLVESGGGLVQPGGSLRLSCAASGFTLS | 2244 |
| | HFRW2 | WVRQAPGKGLEWVA | 2245 |
| | HFRW3 | RFTISRDNANNSLYLQMNNLRAEDTAVYFCAR | 2246 |
| | HFRW4 | WGQGTLVTVSS | 2247 |
| | Light Chain | SYELTQPPSVSVSPGQTASITCSGDKLGDTYACWYQQKPGQSPVLVIY | 2248 |
| | | QDSKRPSGIPERFSGSNSGNTATLTISGTQAMDEADYYCQAWDSSTSV | |
| | | VFGGGTKLTVLGQPKAAPSVTLFPPSSEELQANKATLVCLISDFYPGA | |
| | | VTVAWKADSSPVKAGVETTTPSKQSNNKYAASS~ | |
| | Light Chain | SYELTQPPSVSVSPGQTASITCSGDKLGDTYACWYQQKPGQSPVLVIY | 2249 |
| | Variable | QDSKRPSGIPERFSGSNSGNTATLTISGTQAMDEADYYCQAWDSSTSV | |
| | Region | VFGGGTKLTVL | |
| | LCDR1 | SGDKLGDTYAC | 2250 |
| | LCDR2 | QDSKRPS | 2251 |
| | LCDR3 | QAWDSSTSVV | 2252 |
| | LFRW1 | SYELTQPPSVSVSPGQTASITC | 2253 |
| | LFRW2 | WYQQKPGQSPVLVIY | 2254 |
| | LFRW3 | GIPERFSGSNSGNTATLTISGTQAMDEADYYC | 2255 |
| | LFRW4 | FGGGTKLTVL | 2256 |
| S210-896 | Heavy Chain | QVQLVESGGGVVQPGRSLRLSCAASGFTFSSYAMHWVRQAPGKGLE | 2257 |
| (Spike) | | WVAVISYDGGNKYYADSVKGRFTISRDNSKNTLYLQMNSLRAEDTA | |
| | | VYYCARGHGNYLTYFDYWGQGTLVTVSSGSASAPTLFPLVSCENSPS | |
| | | DTSSV | |
| | Heavy Chain | QVQLVESGGGVVQPGRSLRLSCAASGFTFSSYAMHWVRQAPGKGLE | 2258 |
| | Variable | WVAVISYDGGNKYYADSVKGRFTISRDNSKNTLYLQMNSLRAEDTA | |
| | Region | VYYCARGHGNYLTYFDYWGQGTLVTVSS | |
| | HCDR1 | SYAMH | 2259 |
| | HCDR2 | VISYDGGNKYYADSVKG | 2260 |
| | HCDR3 | GHGNYLTYFDY | 2261 |
| | HFRW1 | QVQLVESGGGVVQPGRSLRLSCAASGFTFS | 2262 |
| | HFRW2 | WVRQAPGKGLEWVA | 2263 |
| | HFRW3 | RFTISRDNSKNTLYLQMNSLRAEDTAVYYCAR | 2264 |
| | HFRW4 | WGQGTLVTVSS | 2265 |
| | Light Chain | EIVLTQSPGTLSLSPGERATLSCRASQSISSNYLAWYQQKPGQAPRLLI | 2266 |
| | | YGASSRATGIPDRFSGSGSGTDFTLTISRLEPEDFAVYYCQQYGSSPLT | |
| | | FGPGTKVDIKRTVAAPSVFIFPPSDEQLKSGTASVVCLLNNFYPREAK | |
| | | VQWKVDN | |
| | Light Chain | EIVLTQSPGTLSLSPGERATLSCRASQSISSNYLAWYQQKPGQAPRLLI | 2267 |
| | Variable | YGASSRATGIPDRFSGSGSGTDFTLTISRLEPEDFAVYYCQQYGSSPLT | |
| | Region | FGPGTKVDIK | |
| | LCDR1 | RASQSISSNYLA | 2268 |
| | LCDR2 | GASSRAT | 2269 |
| | LCDR3 | QQYGSSPLT | 2270 |
| | LFRW1 | EIVLTQSPGTLSLSPGERATLSC | 2271 |
| | LFRW2 | WYQQKPGQAPRLLIY | 2272 |
| | LFRW3 | GIPDRFSGSGSGTDFTLTISRLEPEDFAVYYC | 2273 |
| | LFRW4 | FGPGTKVDIK | 2274 |
| S2141-113 | Heavy Chain | EVQLVESGGGLVQPGGSLRLSCAASGFTFSSSWIHWVRQAPGKGLV | 2275 |
| (Spike/ | | WVSRINSDGSSTTYADSVKGRFTISRDNAKNTLFLQMNSLRAEDTAV | |
| RBD) | | YYCARAEWLRGQFDYWGQGTLVTVSSPPTKAPDVFPIISGCRHPKDN | |
| | | SPVVLACLITGYHPTSVTVTWYMGTQSQPQRTFPEIQRRDSYYMTSSQ | |
| | | LSTPLQQWRQGEYKCVVQ~ | |
| | Heavy Chain | EVQLVESGGGLVQPGGSLRLSCAASGFTFSSSWIHWVRQAPGKGLV | 2276 |
| | Variable | WVSRINSDGSSTTYADSVKGRFTISRDNAKNTLFLQMNSLRAEDTAV | |
| | Region | YYCARAEWLRGQFDYWGQGTLVTVSS | |
| | HCDR1 | SSWIH | 2277 |
| | HCDR2 | RINSDGSSTTYADSVKG | 2278 |
| | HCDR3 | AEWLRGQFDY | 2279 |
| | HFRW1 | EVQLVESGGGLVQPGGSLRLSCAASGFTFS | 2280 |
| | HFRW2 | WVRQAPGKGLVWVS | 2281 |
| | HFRW3 | RFTISRDNAKNTLFLQMNSLRAEDTAVYYCAR | 2282 |
| | HFRW4 | WGQGTLVTVSS | 2283 |
| | Light Chain | NFMLTQPHSVSESPGKTVTISCTGSSGSIASNYVQWYQQRPGSAPTTVI | 2284 |
| | | YEDNQRPSGVPDRFSGSIDSSSNSASLTISGLKTEDEADYYCQSYDTSN | |
| | | HVVFGGGTKLTVLGQPKAAPSVTLFPPSSEELQANKATLVCLISDFYP | |
| | | GAVTVAWKADSSPVKAGVETTTPSKQSNNKYAASSYLSLTPEQWKS | |
| | | H~ | |
| | Light Chain | NFMLTQPHSVSESPGKTVTISCTGSSGSIASNYVQWYQQRPGSAPTTVI | 2285 |
| | Variable | YEDNQRPSGVPDRFSGSIDSSSNSASLTISGLKTEDEADYYCQSYDTSN | |
| | Region | HVVFGGGTKLTVL | |
| | LCDR1 | TGSSGSIASNYVQ | 2286 |
| | LCDR2 | EDNQRPS | 2287 |
| | LCDR3 | QSYDTSNHVV | 2288 |
| | LFRW1 | NFMLTQPHSVSESPGKTVTISC | 2289 |
| | LFRW2 | WYQQRPGSAPTTVIY | 2290 |
| | LFRW3 | GVPDRFSGSIDSSSNSASLTISGLKTEDEADYYC | 2291 |
| | LFRW4 | FGGGTKLTVL | 2292 |
| S2141-126 | Heavy Chain | EVQLVQSGAEVKNPGESLKISCKGSGYRFTTYWIGWVRQMPGKGLE | 2293 |
| (Spike/ | | WMGIIYPGDSDTRYSPSFEGQVTISADKSISTAYLQWSSLKASDTAMY | |
| RBD) | | YCARHPLGLGGSIDYWGQGTLVTVSSASTKGPSVFPLAPSSKSTSGGT | |
| | | AALGCLVKDYFPEPVTVSWNSGALTSGVHTFPAVLQSSGLYSLSSVV | |
| | | TVPSSSLGTQTYICNVNHKPTLVLLGL*F~ | |
| | Heavy Chain | EVQLVQSGAEVKNPGESLKISCKGSGYRFTTYWIGWVRQMPGKGLE | 2294 |
| | Variable | WMGIIYPGDSDTRYSPSFEGQVTISADKSISTAYLQWSSLKASDTAMY | |
| | Region | YCARHPLGLGGSIDYWGQGTLVTVSS | |
| | HCDR1 | TYWIG | 2295 |
| | HCDR2 | IIYPGDSDTRYSPSFEG | 2296 |
| | HCDR3 | HPLGLGGSIDY | 2297 |
| | HFRW1 | EVQLVQSGAEVKNPGESLKISCKGSGYRFT | 2298 |
| | HFRW2 | WVRQMPGKGLEWMG | 2299 |
| | HFRW3 | QVTISADKSISTAYLQWSSLKASDTAMYYCAR | 2300 |
| | HFRW4 | WGQGTLVTVSS | 2301 |
| | Light Chain | DIQMTQSPSTLSASVGDRVTITCRASQSISSWLAWYQQKPGKAPKLLI | 2302 |
| | | YDASSLESGVPSRFSGSGSGTEFTLTISSLQPDDFATYYCQQYNSHWT | |
| | | FGQGTKVEIKRTVAAPSVFIFPPSDEQLKSGTASVVCLLNNFYPREAK | |
| | | VQWKVDNALQSGNSQESVTEQDSKDSTYSLSSTLTLSKADYE~ | |
| | Light Chain | DIQMTQSPSTLSASVGDRVTITCRASQSISSWLAWYQQKPGKAPKLLI | 2303 |
| | Variable | YDASSLESGVPSRFSGSGSGTEFTLTISSLQPDDFATYYCQQYNSHWT | |
| | Region | FGQGTKVEIK | |
| | LCDR1 | RASQSISSWLA | 2304 |
| | LCDR2 | DASSLES | 2305 |
| | LCDR3 | QQYNSHWT | 2306 |
| | LFRW1 | DIQMTQSPSTLSASVGDRVTITC | 2307 |
| | LFRW2 | WYQQKPGKAPKLLIY | 2308 |
| | LFRW3 | GVPSRFSGSGSGTEFTLTISSLQPDDFATYYC | 2309 |
| | LFRW4 | FGQGTKVEIK | 2310 |
| S2141-16 | Heavy Chain | QVQLQQWGAGLLKPSETLSRTCAVYGGSFSGYYWSWIRQTPGKGLE | 2311 |
| (Spike) | | WIGEINHDGSTIYNPSLKSRVTISIDTSKNQFSLQLSSVTAADTAVYYC | |
| | | ARGSNPGDYWGQGALVTVSSAPTKAPDVFPIISGCRHPKDNSPVVLA | |
| | | CLITGYHPT~ | |
| | Heavy Chain | QVQLQQWGAGLLKPSETLSRTCAVYGGSFSGYYWSWIRQTPGKGLE | 2312 |
| | Variable | WIGEINHDGSTIYNPSLKSRVTISIDTSKNQFSLQLSSVTAADTAVYYC | |
| | Region | ARGSNPGDYWGQGALVTVSS | |
| | HCDR1 | GYYWS | 2313 |
| | HCDR2 | EINHDGSTIYNPSLKS | 2314 |
| | HCDR3 | GSNPGDY | 2315 |
| | HFRW1 | QVQLQQWGAGLLKPSETLSRTCAVYGGSFS | 2316 |
| | HFRW2 | WIRQTPGKGLEWIG | 2317 |
| | HFRW3 | RVTISIDTSKNQFSLQLSSVTAADTAVYYCAR | 2318 |
| | HFRW4 | WGQGALVTVSS | 2319 |
| | Light Chain | SYELTQSLSVSVALGQTARIPCGGNNIGSKNVHWYQQKPGQAPVLVI | 2320 |
| | | YSDRNRPSGIPERFSGSNSGNTATLTISRAQAGDEADYYCQVWDSSSV | |
| | | VFGGGTKLTVLRQPKAAPSVTLFPPSSEELQANKATLVCLISDFYPGA | |
| | | VTVAWKADSSPVKAGVETTTPSKQSNNKYAASSYLSLTPEQWKSH~ | |
| | Light Chain | SYELTQSLSVSVALGQTARIPCGGNNIGSKNVHWYQQKPGQAPVLVI | 2321 |
| | Variable | YSDRNRPSGIPERFSGSNSGNTATLTISRAQAGDEADYYCQVWDSSSV | |
| | Region | VFGGGTKLTVL | |
| | LCDR1 | GGNNIGSKNVH | 2322 |
| | LCDR2 | SDRNRPS | 2323 |
| | LCDR3 | QVWDSSSVV | 2324 |
| | LFRW1 | SYELTQSLSVSVALGQTARIPC | 2325 |
| | LFRW2 | WYQQKPGQAPVLVIY | 2326 |
| | LFRW3 | GIPERFSGSNSGNTATLTISRAQAGDEADYYC | 2327 |
| | LFRW4 | FGGGTKLTVL | 2328 |
| S2141-62 | Heavy Chain | QVHLQESGPGLVKPSQTLSLTCTVSGVSITTSGSYWSWIRQCPGKGLE | 2329 |
| (Spike/ | | WIGYIYSTGTTYYSPSLKSRLTISLDTSRNQFSLNLSSVTAADTAVFFC | |
| RBD) | | ARKTYMDYFDYWGQGALITVSSASTKGPSVFPLAPSSKSTSGGTAAL | |
| | | GCLVKDYFPEPVTVSWNSGALTSGVHTFPAVLQSSG | |
| | Heavy Chain | QVHLQESGPGLVKPSQTLSLTCTVSGVSITTSGSYWSWIRQCPGKGLE | 2330 |
| | Variable | WIGYIYSTGTTYYSPSLKSRLTISLDTSRNQFSLNLSSVTAADTAVFFC | |
| | Region | ARKTYMDYFDYWGQGALITVSS | |
| | HCDR1 | TSGSYWS | 2331 |
| | HCDR2 | YIYSTGTTYYSPSLKS | 2332 |
| | HCDR3 | KTYMDYFDY | 2333 |
| | HFRW1 | QVHLQESGPGLVKPSQTLSLTCTVSGVSIT | 2334 |
| | HFRW2 | WIRQCPGKGLEWIG | 2335 |
| | HFRW3 | RLTISLDTSRNQFSLNLSSVTAADTAVFFCAR | 2336 |
| | HFRW4 | WGQGALITVSS | 2337 |
| | Light Chain | QSALTQPTSVSGSPGQSITISCTGTSSDVGRYNLVSWYQQYPGKAPKLI | 2338 |
| | | IFEVSKRPSGVSDRFSASKSGNTASLTISGLQADDEADYYCCTYALTFL | |
| | | FGGGTKVTVLGQPKAAPSVTLFPPSSEELQANKATLVCLISDFYPGAV | |
| | | TVAWKADSSPVKAGVETTTPSKQSNNKYAASSYLSLTPEQWKSH | |
| | Light Chain | QSALTQPTSVSGSPGQSITISCTGTSSDVGRYNLVSWYQQYPGKAPKLI | 2339 |
| | Variable | IFEVSKRPSGVSDRFSASKSGNTASLTISGLQADDEADYYCCTYALTFL | |
| | Region | FGGGTKVTVL | |
| | LCDR1 | TGTSSDVGRYNLVS | 2340 |
| | LCDR2 | EVSKRPS | 2341 |
| | LCDR3 | CTYALTFL | 2342 |
| | LFRW1 | QSALTQPTSVSGSPGQSITISC | 2343 |
| | LFRW2 | WYQQYPGKAPKLIIF | 2344 |
| | LFRW3 | GVSDRFSASKSGNTASLTISGLQADDEADYYC | 2345 |
| | LFRW4 | FGGGTKVTVL | 2346 |
| S2141-63 | Heavy Chain | EVQLLESGGGLVQPGGSLRLSCAASGFTFYDYAMNWVRQTPGEGLE | 2347 |
| (Spike/ | | WVSAISGSGDPTYYADSVNGRFTISRDNSKNTLYLQMNSLRAEDTAI | |
| RBD) | | YYCAKDMEDFGFSWGQGTLVTVSSAPTKAPDVFPIISGCRHPKDNSP | |
| | | VVLACLITGYHPTSVTVTWYM~ | |
| | Heavy Chain | EVQLLESGGGLVQPGGSLRLSCAASGFTFYDYAMNWVRQTPGEGLE | 2348 |
| | Variable | WVSAISGSGDPTYYADSVNGRFTISRDNSKNTLYLQMNSLRAEDTAI | |
| | Region | YYCAKDMEDFGFSWGQGTLVTVSS | |
| | HCDR1 | DYAMN | 2349 |
| | HCDR2 | AISGSGDPTYYADSVNG | 2350 |
| | HCDR3 | DMEDFGFS | 2351 |
| | HFRW1 | EVQLLESGGGLVQPGGSLRLSCAASGFTFY | 2352 |
| | HFRW2 | WVRQTPGEGLEWVS | 2353 |
| | HFRW3 | RFTISRDNSKNTLYLQMNSLRAEDTAIYYCAK | 2354 |
| | HFRW4 | WGQGTLVTVSS | 2355 |
| | Light Chain | DIQMTQSPSSLSASVGDRVTITCRSGQSISTYLNWYQQKPGKAPKLLI | 2356 |
| | | YASSSLQSGVPSRFSGSGSGTDFTLTISSLQPEDFATYYCQQSFLPPRTF | |
| | | GQGTKLEIKRTVAAPSVFIFPPSDEQLKSGTASVVCLLNNFYPREAKV | |
| | | QWKVDNALQSGNSQESVTEQDSKDSTYSLSSTLTLSKADYE~ | |
| | Light Chain | DIQMTQSPSSLSASVGDRVTITCRSGQSISTYLNWYQQKPGKAPKLLI | 2357 |
| | Variable | YASSSLQSGVPSRFSGSGSGTDFTLTISSLQPEDFATYYCQQSFLPPRTF | |
| | Region | GQGTKLEIK | |
| | LCDR1 | RSGQSISTYLN | 2358 |
| | LCDR2 | ASSSLQS | 2359 |
| | LCDR3 | QQSFLPPRT | 2360 |
| | LFRW1 | DIQMTQSPSSLSASVGDRVTITC | 2361 |
| | LFRW2 | WYQQKPGKAPKLLIY | 2362 |
| | LFRW3 | GVPSRFSGSGSGTDFTLTISSLQPEDFATYYC | 2363 |
| | LFRW4 | FGQGTKLEIK | 2364 |
| S2141-65 | Heavy Chain | DVQLVQSGAEVTKPGESLKISCKGSGYSFTTYWIGWVRQMPGKGLE | 2365 |
| (Spike) | | WMGIIYPGDSDTRYSPSFQGQVTISVDKSISTAYLQWSSLKASDTAMY | |
| | | YCARQFCGGDCPFDYWGRGTLVTVSSASTKGPSVFPLAPCSRSTSGG | |
| | | TAALGCLVKDYFPEPVTVSWNSGALTSGVHTFPAVLQSSGLYSLSSV | |
| | | VTVPSSSLGTQTYTCNVNHKPSNTKVD~ | |
| | Heavy Chain | DVQLVQSGAEVTKPGESLKISCKGSGYSFTTYWIGWVRQMPGKGLE | 2366 |
| | Variable | WMGIIYPGDSDTRYSPSFQGQVTISVDKSISTAYLQWSSLKASDTAMY | |
| | Region | YCARQFCGGDCPFDYWGRGTLVTVSS | |
| | HCDR1 | TYWIG | 2367 |
| | HCDR2 | IIYPGDSDTRYSPSFQG | 2368 |
| | HCDR3 | QFCGGDCPFDY | 2369 |
| | HFRW1 | DVQLVQSGAEVTKPGESLKISCKGSGYSFT | 2370 |
| | HFRW2 | WVRQMPGKGLEWMG | 2371 |
| | HFRW3 | QVTISVDKSISTAYLQWSSLKASDTAMYYCAR | 2372 |
| | HFRW4 | WGRGTLVTVSS | 2373 |
| | Light Chain | DIQMTQSPSTLSASVGDRVTITCRASQSISSWLAWYQQKPGKAPKLLI | 2374 |
| | | YDASSLEGGVPSRFSGSGSGTEFTLTISSLQPDDFATYYCQQYNSYPRT | |
| | | FGQGTKVEIKRTVAAPSVFIFPPSDEQLKSGTASVVCLLNNFYPREAK | |
| | | VQWKVDNALQSGNSQESVTEQDSKDSTYSLSSTLTLSKADYE~ | |
| | Light Chain | DIQMTQSPSTLSASVGDRVTITCRASQSISSWLAWYQQKPGKAPKLLI | 2375 |
| | Variable | YDASSLEGGVPSRFSGSGSGTEFTLTISSLQPDDFATYYCQQYNSYPRT | |
| | Region | FGQGTKVEIK | |
| | LCDR1 | RASQSISSWLA | 2376 |
| | LCDR2 | DASSLEG | 2377 |
| | LCDR3 | QQYNSYPRT | 2378 |
| | LFRW1 | DIQMTQSPSTLSASVGDRVTITC | 2379 |
| | LFRW2 | WYQQKPGKAPKLLIY | 2380 |
| | LFRW3 | GVPSRFSGSGSGTEFTLTISSLQPDDFATYYC | 2381 |
| | LFRW4 | FGQGTKVEIK | 2382 |
| S2141-97 | Heavy Chain | QVQLVQSGAEVKKPGASVKVSCKASGYTFTRYGMHWVRQAPGQRL | 2383 |
| (Spike/ | | KWMGWINAGNGNTKYSQKFQGRLTISRDTSASTAYMEVSSLRSEDT | |
| RBD) | | AVYYCARSGIAAAGSKVIYYYDMDVWGQGTTVTVSSAPTKAPDVFPI | |
| | | ISGCRHPKDNSPVVLACLITGYHPTSVTVTWYMGTQSQPQRTFPEIQR | |
| | | RDSYYMTSSQLSTPLQQWRQGEYKCVVQ~ | |
| | Heavy Chain | QVQLVQSGAEVKKPGASVKVSCKASGYTFTRYGMHWVRQAPGQRL | 2384 |
| | Variable | KWMGWINAGNGNTKYSQKFQGRLTISRDTSASTAYMEVSSLRSEDT | |
| | Region | AVYYCARSGIAAAGSKVIYYYDMDVWGQGTTVTVSS | |
| | HCDR1 | RYGMH | 2385 |
| | HCDR2 | WINAGNGNTKYSQKFQG | 2386 |
| | HCDR3 | SGIAAAGSKVIYYYDMDV | 2387 |
| | HFRW1 | QVQLVQSGAEVKKPGASVKVSCKASGYTFT | 2388 |
| | HFRW2 | WVRQAPGQRLKWMG | 2389 |
| | HFRW3 | RLTISRDTSASTAYMEVSSLRSEDTAVYYCAR | 2390 |
| | HFRW4 | WGQGTTVTVSS | 2391 |
| | Light Chain | EIVLTQSPGTLSLSPGERATLSCRASQRVSSSYIAWYQQKPGQAPRLLI | 2392 |
| | | FGTSSRATGIPDRFSGSGSGTDFTLTISRLEPEDFALYYCQQYGSSPYTF | |
| | | GQGTKLEIKRTVAAPSVFIFPPSDEQLKSGTASVVCLLNNFYPREAKV | |
| | | QWKVDNALQSGNSQESVTEQDSKDSTYSLSSTLTLSKADYE~ | |
| | Light Chain | EIVLTQSPGTLSLSPGERATLSCRASQRVSSSYIAWYQQKPGQAPRLLI | 2393 |
| | Variable | FGTSSRATGIPDRFSGSGSGTDFTLTISRLEPEDFALYYCQQYGSSPYTF | |
| | Region | GQGTKLEIK | |
| | LCDR1 | RASQRVSSSYIA | 2394 |
| | LCDR2 | GTSSRAT | 2395 |
| | LCDR3 | QQYGSSPYT | 2396 |
| | LFRW1 | EIVLTQSPGTLSLSPGERATLSC | 2397 |
| | LFRW2 | WYQQKPGQAPRLLIF | 2398 |
| | LFRW3 | GIPDRFSGSGSGTDFTLTISRLEPEDFALYYC | 2399 |
| | LFRW4 | FGQGTKLEIK | 2400 |
| S24_342 | Heavy Chain | QVQLVQSGAEVKMPGASVIVSCKASGYTFSTYYIHWVRQAPGQGLE | 2401 |
| (Spike/ | | WMGRITPRDGDTTYAQVLQGRVTLTRDTSASTAYMELSSLTYEDTA | |
| RBD) | | VYYCARDGHHWDFDFWGRGTLVAVSSASTKGPSVFPLAPCSRSTSES | |
| | | TAALGCLVKDYFPEPVTVSWNSGALTSGVHTFPAVLQSS | |
| | Heavy Chain | QVQLVQSGAEVKMPGASVIVSCKASGYTFSTYYIHWVRQAPGQGLE | 2402 |
| | Variable | WMGRITPRDGDTTYAQVLQGRVTLTRDTSASTAYMELSSLTYEDTA | |
| | Region | VYYCARDGHHWDFDFWGRGTLVAVSS | |
| | HCDR1 | TYYIH | 2403 |
| | HCDR2 | RITPRDGDTTYAQVLQG | 2404 |
| | HCDR3 | DGHHWDFDF | 2405 |
| | HFRW1 | QVQLVQSGAEVKMPGASVIVSCKASGYTFS | 2406 |
| | HFRW2 | WVRQAPGQGLEWMG | 2407 |
| | HFRW3 | RVTLTRDTSASTAYMELSSLTYEDTAVYYCAR | 2408 |
| | HFRW4 | WGRGTLVAVSS | 2409 |
| | Light Chain | HSALTQPPSASGSPGQSVTISCTGTSSDVGGYNHVSWYQQHPGKAPK | 2410 |
| | | LMVYEVNQRPSGVPDRFTGSKSGNTASLTVSGLQAEDEADYYCNSY | |
| | | TDRNKWVFGGGTRLTVLGQPKAAPSVTLFPPSSEELQANKATLVCLIS | |
| | | DFYPGAVTVAWKADSSPVKAGVETTTPSKQSNNKYAASS~ | |
| | Light Chain | HSALTQPPSASGSPGQSVTISCTGTSSDVGGYNHVSWYQQHPGKAPK | 2411 |
| | Variable | LMVYEVNQRPSGVPDRFTGSKSGNTASLTVSGLQAEDEADYYCNSY | |
| | Region | TDRNKWVFGGGTRLTVL | |
| | LCDR1 | TGTSSDVGGYNHVS | 2412 |
| | LCDR2 | EVNQRPS | 2413 |
| | LCDR3 | NSYTDRNKWV | 2414 |
| | LFRW1 | HSALTQPPSASGSPGQSVTISC | 2415 |
| | LFRW2 | WYQQHPGKAPKLMVY | 2416 |
| | LFRW3 | GVPDRFTGSKSGNTASLTVSGLQAEDEADYYC | 2417 |
| | LFRW4 | FGGGTRLTVL | 2418 |
| S24-1047 | Heavy Chain | QVQLKQSGAEVKEPGGSVKLSCKASGYTFTSRYIHWVRQAPGQGLE | 2419 |
| (Spike/ | | WVGRLIPSDGGTTYAQKFRGRVTMTSDTSATTAYMELSSLGSGDTAV | |
| RBD) | | YYCARDGTHWDFDFWGQGTLVTVSSASPTSPKVFPLSLDSTPQDGNV | |
| | | VVACLVQGFFPQEPLSVTWSESGQNVTARNF~ | |
| | Heavy Chain | QVQLKQSGAEVKEPGGSVKLSCKASGYTFTSRYIHWVRQAPGQGLE | 2420 |
| | Variable | WVGRLIPSDGGTTYAQKFRGRVTMTSDTSATTAYMELSSLGSGDTAV | |
| | Region | YYCARDGTHWDFDFWGQGTLVTVSS | |
| | HCDR1 | SRYIH | 2421 |
| | HCDR2 | RLIPSDGGTTYAQKFRG | 2422 |
| | HCDR3 | DGTHWDFDF | 2423 |
| | HFRW1 | QVQLKQSGAEVKEPGGSVKLSCKASGYTFT | 2424 |
| | HFRW2 | WVRQAPGQGLEWVG | 2425 |
| | HFRW3 | RVTMTSDTSATTAYMELSSLGSGDTAVYYCAR | 2426 |
| | HFRW4 | WGQGTLVTVSS | 2427 |
| | Light Chain | HSALTQPPSASGSPGQSVTISCTGTSDDVGGYNHVSWYQQHPGKAPK | 2428 |
| | | LVIYEVTERPSGVPDRFTGSKSGNTASLTVSGLQAEDEADYYCNSYK | |
| | | RGNTWVFGGGTRLTVLGQPKAAPSVTLFPPSSEELQANKATLVCLISD | |
| | | FYPGAVTVAWKADSSPVKAGVETTTPSKQSNNKYAASS~ | |
| | Light Chain | HSALTQPPSASGSPGQSVTISCTGTSDDVGGYNHVSWYQQHPGKAPK | 2429 |
| | Variable | LVIYEVTERPSGVPDRFTGSKSGNTASLTVSGLQAEDEADYYCNSYK | |
| | Region | RGNTWVFGGGTRLTVL | |
| | LCDR1 | TGTSDDVGGYNHVS | 2430 |
| | LCDR2 | EVTERPS | 2431 |
| | LCDR3 | NSYKRGNTWV | 2432 |
| | LFRW1 | HSALTQPPSASGSPGQSVTISC | 2433 |
| | LFRW2 | WYQQHPGKAPKLVIY | 2434 |
| | LFRW3 | GVPDRFTGSKSGNTASLTVSGLQAEDEADYYC | 2435 |
| | LFRW4 | FGGGTRLTVL | 2436 |
| S24-223 | Heavy Chain | QITLKESGPTLVKPTQTLTLTCTFSGFSLNTSGVGVGWIRQPPGKALE | 2437 |
| (Spike/ | | WLALIYWDDDKRYSPSLKSRLTITKDTSKNQVVLTMTNMDPVDTAT | |
| RBD) | | YYCAHHTIVPIFDYWGQGTLVTVSSGSASAPTLFPLVSCENSPSDTSSV | |
| | Heavy Chain | QITLKESGPTLVKPTQTLTLTCTFSGFSLNTSGVGVGWIRQPPGKALE | 2438 |
| | Variable | WLALIYWDDDKRYSPSLKSRLTITKDTSKNQVVLTMTNMDPVDTAT | |
| | Region | YYCAHHTIVPIFDYWGQGTLVTVSS | |
| | HCDR1 | TSGVGVG | 2439 |
| | HCDR2 | LIYWDDDKRYSPSLKS | 2440 |
| | HCDR3 | HTIVPIFDY | 2441 |
| | HFRW1 | QITLKESGPTLVKPTQTLTLTCTFSGFSLN | 2442 |
| | HFRW2 | WIRQPPGKALEWLA | 2443 |
| | HFRW3 | RLTITKDTSKNQVVLTMTNMDPVDTATYYCAH | 2444 |
| | HFRW4 | WGQGTLVTVSS | 2445 |
| | Light Chain | QSALTQPASVSGSPGQSITISCTGTSSDVGGYNYVSWYQQHPGKAPKL | 2446 |
| | | MIYDVSNRPSGVSNRFSGSKSGNTASLTISGLQAEDEADYYCNSYTSS | |
| | | STLVVFGGGTKLTVLGQPKAAPSVTLFPPSSEELQANKATLVCLISDF | |
| | | YPGAVTVAWKADSSPVKAGVETTTPSKQSNNKYAASSYLSLT~ | |
| | Light Chain | QSALTQPASVSGSPGQSITISCTGTSSDVGGYNYVSWYQQHPGKAPKL | 2447 |
| | Variable | MIYDVSNRPSGVSNRFSGSKSGNTASLTISGLQAEDEADYYCNSYTSS | |
| | Region | STLVVFGGGTKLTVL | |
| | LCDR1 | TGTSSDVGGYNYVS | 2448 |
| | LCDR2 | DVSNRPS | 2449 |
| | LCDR3 | NSYTSSSTLVV | 2450 |
| | LFRW1 | QSALTQPASVSGSPGQSITISC | 2451 |
| | LFRW2 | WYQQHPGKAPKLMIY | 2452 |
| | LFRW3 | GVSNRFSGSKSGNTASLTISGLQAEDEADYYC | 2453 |
| | LFRW4 | FGGGTKLTVL | 2454 |
| S24-237 | Heavy Chain | QVQLQESGPGLVKPSGTLSLTCSVSGGSINSSFWSWIRQPPGKGLEWI | 2455 |
| (Spike/ | | GYIYYRGSTNYNPSLKSRVTISVDTSNNQFSLKLTSMTAADSAVYYC | |
| RBD) | | ARETRYNWFDSWGQGTRVTVSSASTKGPSVFPLAPCSRSTSESTAAL | |
| | | GCLVKDYFPEPVTVSWNSGALTSGVHTFPAVLQSSG | |
| | Heavy Chain | QVQLQESGPGLVKPSGTLSLTCSVSGGSINSSFWSWIRQPPGKGLEWI | 2456 |
| | Variable | GYIYYRGSTNYNPSLKSRVTISVDTSNNQFSLKLTSMTAADSAVYYC | |
| | Region | ARETRYNWFDSWGQGTRVTVSS | |
| | HCDR1 | SSFWS | 2457 |
| | HCDR2 | YIYYRGSTNYNPSLKS | 2458 |
| | HCDR3 | ETRYNWFDS | 2459 |
| | HFRW1 | QVQLQESGPGLVKPSGTLSLTCSVSGGSIN | 2460 |
| | HFRW2 | WIRQPPGKGLEWIG | 2461 |
| | HFRW3 | RVTISVDTSNNQFSLKLTSMTAADSAVYYCAR | 2462 |
| | HFRW4 | WGQGTRVTVSS | 2463 |
| | Light Chain | DIVMTQSPDSLAVSLGERATINCKSSQTVSYTSNNKNYLAWYQQKPG | 2464 |
| | | QPPNLLIYWASTRESGVPDRFSGSGSGTDFTLTINSLQAEDVAVYYCQ | |
| | | QYYTTPWTFGQGTKVEIKRTVAAPSVFIFPPSDEQLKSGTASVVCLLN | |
| | | NFYPREAKVQWKVDN | |
| | Light Chain | DIVMTQSPDSLAVSLGERATINCKSSQTVSYTSNNKNYLAWYQQKPG | 2465 |
| | Variable | QPPNLLIYWASTRESGVPDRFSGSGSGTDFTLTINSLQAEDVAVYYCQ | |
| | Region | QYYTTPWTFGQGTKVEIK | |
| | LCDR1 | KSSQTVSYTSNNKNYLA | 2466 |
| | LCDR2 | WASTRES | 2467 |
| | LCDR3 | QQYYTTPWT | 2468 |
| | LFRW1 | DIVMTQSPDSLAVSLGERATINC | 2469 |
| | LFRW2 | WYQQKPGQPPNLLIY | 2470 |
| | LFRW3 | GVPDRFSGSGSGTDFTLTINSLQAEDVAVYYC | 2471 |
| | LFRW4 | FGQGTKVEIK | 2472 |
| S305-1456 | Heavy Chain | QVQLVQSGAEVKKPGASVKVSCKVSGYTLTELSMHWVRQAPGKGL | 2473 |
| (Spike) | | EWMGGFDPEDAETIYAQKFQGRVTMTEDTSTDTAYMELSSLRSEDT | |
| | | AVYYCATGGFPVNSLYDILTGYLDYWGQGTLVTVSSASTKGPSVFPL | |
| | | APCSRSTSESTAALGCLVKDYFPEPVTVSWNSGALTSGVHTFPAVLQS | |
| | | SG | |
| | Heavy Chain | QVQLVQSGAEVKKPGASVKVSCKVSGYTLTELSMHWVRQAPGKGL | 2474 |
| | Variable | EWMGGFDPEDAETIYAQKFQGRVTMTEDTSTDTAYMELSSLRSEDT | |
| | Region | AVYYCATGGFPVNSLYDILTGYLDYWGQGTLVTVSS | |
| | HCDR1 | ELSMH | 2475 |
| | HCDR2 | GFDPEDAETIYAQKFQG | 2476 |
| | HCDR3 | GGFPVNSLYDILTGYLDY | 2477 |
| | HFRW1 | QVQLVQSGAEVKKPGASVKVSCKVSGYTLT | 2478 |
| | HFRW2 | WVRQAPGKGLEWMG | 2479 |
| | HFRW3 | RVTMTEDTSTDTAYMELSSLRSEDTAVYYCAT | 2480 |
| | HFRW4 | WGQGTLVTVSS | 2481 |
| | Light Chain | EIVMTQSPATLSVSPGERATLSCRASQNVSSNLAWYQQKPGQAPRLLI | 2482 |
| | | YGASTRATGIPARFSGSGSGTEFTLTISSLQSEDFAVYYCQQYNNWPH | |
| | | TFGPGTKVDIKRTVAAPSVFIFPPSDEQLKSGTASVVCLLNNFYPREAK | |
| | | VQWKVDN~ | |
| | Light Chain | EIVMTQSPATLSVSPGERATLSCRASQNVSSNLAWYQQKPGQAPRLLI | 2483 |
| | Variable | YGASTRATGIPARFSGSGSGTEFTLTISSLQSEDFAVYYCQQYNNWPH | |
| | Region | TFGPGTKVDIK | |
| | LCDR1 | RASQNVSSNLA | 2484 |
| | LCDR2 | GASTRAT | 2485 |
| | LCDR3 | QQYNNWPHT | 2486 |
| | LFRW1 | EIVMTQSPATLSVSPGERATLSC | 2487 |
| | LFRW2 | WYQQKPGQAPRLLIY | 2488 |
| | LFRW3 | GIPARFSGSGSGTEFTLTISSLQSEDFAVYYC | 2489 |
| | LFRW4 | FGPGTKVDIK | 2490 |
| S305-223 | Heavy Chain | QVQLVESGGGVVQPGRSLRLSCAASGFTFRNFGMHWVRQAPGKGLE | 2491 |
| (Spike) | | WVAFIWTAESDKFYADSVKGRFTVSRDNSKNTLYLEMNSLRAEDTA | |
| | | VYYCTKAMDVWGRGTTVTVSSASPTSPKVFPLSLCSTQPDGNVVIAC | |
| | | LVQGFFPQEPLSVTWSESGQGVTARNF~ | |
| | Heavy Chain | QVQLVESGGGVVQPGRSLRLSCAASGFTFRNFGMHWVRQAPGKGLE | 2492 |
| | Variable | WVAFIWTAESDKFYADSVKGRFTVSRDNSKNTLYLEMNSLRAEDTA | |
| | Region | VYYCTKAMDVWGRGTTVTVSS | |
| | HCDR1 | NFGMH | 2493 |
| | HCDR2 | FIWTAESDKFYADSVKG | 2494 |
| | HCDR3 | AMDV | 2495 |
| | HFRW1 | QVQLVESGGGVVQPGRSLRLSCAASGFTFR | 2496 |
| | HFRW2 | WVRQAPGKGLEWVA | 2497 |
| | HFRW3 | RFTVSRDNSKNTLYLEMNSLRAEDTAVYYCTK | 2498 |
| | HFRW4 | WGRGTTVTVSS | 2499 |
| | Light Chain | EIVLTQSPATLSLSPGERATLSCRASQSVSTSLAWYQQKCGQAPRLLIY | 2500 |
| | | DASNRATGIPARFSGSGSGTDFTLTISSLEPEDFAVYYCQQRGNWPFTF | |
| | | GPGTRVDIKRTVAAPSVFIFPPSDEQLKSGTASVVCLLNNFYPREAKV | |
| | | QWKVDN~ | |
| | Light Chain | EIVLTQSPATLSLSPGERATLSCRASQSVSTSLAWYQQKCGQAPRLLIY | 2501 |
| | Variable | DASNRATGIPARFSGSGSGTDFTLTISSLEPEDFAVYYCQQRGNWPFTF | |
| | Region | GPGTRVDIK | |
| | LCDR1 | RASQSVSTSLA | 2502 |
| | LCDR2 | DASNRAT | 2503 |
| | LCDR3 | QQRGNWPFT | 2504 |
| | LFRW1 | EIVLTQSPATLSLSPGERATLSC | 2505 |
| | LFRW2 | WYQQKCGQAPRLLIY | 2506 |
| | LFRW3 | GIPARFSGSGSGTDFTLTISSLEPEDFAVYYC | 2507 |
| | LFRW4 | FGPGTRVDIK | 2508 |
| S305-399 | Heavy Chain | QVQLVQSGAEVKKPGASVKVSCKVSGYTLTELSMHWVRQAPGKGL | 2509 |
| (Spike) | | EWMGGFDPEDGETIYAQKFQGRVTMTEDTSTDTAYMELSSLRSEDT | |
| | | AVYYCATGGLGCSNGVCNNWFDPWGLGTLVTVSSGSASAPTLFPLV | |
| | | SCENSPSDTSSV | |
| | Heavy Chain | QVQLVQSGAEVKKPGASVKVSCKVSGYTLTELSMHWVRQAPGKGL | 2510 |
| | Variable | EWMGGFDPEDGETIYAQKFQGRVTMTEDTSTDTAYMELSSLRSEDT | |
| | Region | AVYYCATGGLGCSNGVCNNWFDPWGLGTLVTVSS | |
| | HCDR1 | ELSMH | 2511 |
| | HCDR2 | GFDPEDGETIYAQKFQG | 2512 |
| | HCDR3 | GGLGCSNGVCNNWFDP | 2513 |
| | HFRW1 | QVQLVQSGAEVKKPGASVKVSCKVSGYTLT | 2514 |
| | HFRW2 | WVRQAPGKGLEWMG | 2515 |
| | HFRW3 | RVTMTEDTSTDTAYMELSSLRSEDTAVYYCAT | 2516 |
| | HFRW4 | WGLGTLVTVSS | 2517 |
| | Light Chain | EIVMTQSPATLSVSPGERATLSCRASQSITSNLAWYQQKPGQAPRLLI | 2518 |
| | | YGASTRATGIPARFSGSGSGTEFTLTISNLQSEDFAVYYCQQYNNWPL | |
| | | TFGQGTKVEIKRTVAAPSVFIFPPSDEQLKSGTASVVCLLNNFYPREA | |
| | | KVQWKVDNALQSGNSQESVTEQDS~ | |
| | Light Chain | EIVMTQSPATLSVSPGERATLSCRASQSITSNLAWYQQKPGQAPRLLI | 2519 |
| | Variable | YGASTRATGIPARFSGSGSGTEFTLTISNLQSEDFAVYYCQQYNNWPL | |
| | Region | TFGQGTKVEIK | |
| | LCDR1 | RASQSITSNLA | 2520 |
| | LCDR2 | GASTRAT | 2521 |
| | LCDR3 | QQYNNWPLT | 2522 |
| | LFRW1 | EIVMTQSPATLSVSPGERATLSC | 2523 |
| | LFRW2 | WYQQKPGQAPRLLIY | 2524 |
| | LFRW3 | GIPARFSGSGSGTEFTLTISNLQSEDFAVYYC | 2525 |
| | LFRW4 | FGQGTKVEIK | 2526 |
| S305-968 | Heavy Chain | EVQLVESGGGLVQPGGSLRLSCAASGFTFSSYWMSWVRQAPGKGLE | 2527 |
| (Spike) | | WVANIKQDGSEKYYVDSVKGRFTISRDNAKNSLYLQMNSLRAEDTA | |
| | | VYYCARDSIAVAGGFDYWGQGTLVTVSSGSASAPTLFPLVSCENSPS | |
| | | DTSSV | |
| | Heavy Chain | EVQLVESGGGLVQPGGSLRLSCAASGFTFSSYWMSWVRQAPGKGLE | 2528 |
| | Variable | WVANIKQDGSEKYYVDSVKGRFTISRDNAKNSLYLQMNSLRAEDTA | |
| | Region | VYYCARDSIAVAGGFDYWGQGTLVTVSS | |
| | HCDR1 | SYWMS | 2529 |
| | HCDR2 | NIKQDGSEKYYVDSVKG | 2530 |
| | HCDR3 | DSIAVAGGFDY | 2531 |
| | HFRW1 | EVQLVESGGGLVQPGGSLRLSCAASGFTFS | 2532 |
| | HFRW2 | WVRQAPGKGLEWVA | 2533 |
| | HFRW3 | RFTISRDNAKNSLYLQMNSLRAEDTAVYYCAR | 2534 |
| | HFRW4 | WGQGTLVTVSS | 2535 |
| | Light Chain | SYELTQPPSVSVSPGQTASITCSGDKLGDKYACWYQQKPGQSPVLVIY | 2536 |
| | | QDSKRPSGIPERFSGSNSGNTATLTISGTQAMDEADYYCQAWDSSTN | |
| | | VVFGGGTKLTVLGQPKAAPSVTLFPPSSEELQANKATLVCLISDFYPG | |
| | | AVTVAWKADSSPVKAGVETTTPSKQSNNKYAASSYLSLTPEQWKSH | |
| | Light Chain | SYELTQPPSVSVSPGQTASITCSGDKLGDKYACWYQQKPGQSPVLVIY | 2537 |
| | Variable | QDSKRPSGIPERFSGSNSGNTATLTISGTQAMDEADYYCQAWDSSTN | |
| | Region | VVFGGGTKLTVL | |
| | LCDR1 | SGDKLGDKYAC | 2538 |
| | LCDR2 | QDSKRPS | 2539 |
| | LCDR3 | QAWDSSTNVV | 2540 |
| | LFRW1 | SYELTQPPSVSVSPGQTASITC | 2541 |
| | LFRW2 | WYQQKPGQSPVLVIY | 2542 |
| | LFRW3 | GIPERFSGSNSGNTATLTISGTQAMDEADYYC | 2543 |
| | LFRW4 | FGGGTKLTVL | 2544 |
| S376-1070 | Heavy Chain | QVQLVESGGGVVQPGRSLRLSCAASGFTFSSYGMHWVRQAPGKGLE | 2545 |
| (Spike) | | WVAVIWYDGSNKYYADSVKGRFTISRDNSKNTLYLQMNSLRAEDTA | |
| | | VYYCARMRPEYSSGFDPWGQGTLVTVSSGSASAPTLFPLVSCENSPSD | |
| | | TSSV | |
| | Heavy Chain | QVQLVESGGGVVQPGRSLRLSCAASGFTFSSYGMHWVRQAPGKGLE | 2546 |
| | Variable | WVAVIWYDGSNKYYADSVKGRFTISRDNSKNTLYLQMNSLRAEDTA | |
| | Region | VYYCARMRPEYSSGFDPWGQGTLVTVSS | |
| | HCDR1 | SYGMH | 2547 |
| | HCDR2 | VIWYDGSNKYYADSVKG | 2548 |
| | HCDR3 | MRPEYSSGFDP | 2549 |
| | HFRW1 | QVQLVESGGGVVQPGRSLRLSCAASGFTFS | 2550 |
| | HFRW2 | WVRQAPGKGLEWVA | 2551 |
| | HFRW3 | RFTISRDNSKNTLYLQMNSLRAEDTAVYYCAR | 2552 |
| | HFRW4 | WGQGTLVTVSS | 2553 |
| | Light Chain | QSALTQPRSVSGSPGQSVTISCTGSSSDVGRYNYVSWYQQHPGKAPK | 2554 |
| | | LMTYDVTRRPSGVPARFSGSKSDNTASLTISGLQAEDEADYYCCSFA | |
| | | GSYTVFGGGTKLTVLGQPKAAPSVTLFPPSSEELQANKATLVCLISDF | |
| | | YPGAVTVAWKADGSPVKVGVETTKPSKQSNNKYAASSYLSLTPEQW | |
| | | KS~ | |
| | Light Chain | QSALTQPRSVSGSPGQSVTISCTGSSSDVGRYNYVSWYQQHPGKAPK | 2555 |
| | Variable | LMTYDVTRRPSGVPARFSGSKSDNTASLTISGLQAEDEADYYCCSFA | |
| | Region | GSYTVFGGGTKLTVL | |
| | LCDR1 | TGSSSDVGRYNYVS | 2556 |
| | LCDR2 | DVTRRPS | 2557 |
| | LCDR3 | CSFAGSYTV | 2558 |
| | LFRW1 | QSALTQPRSVSGSPGQSVTISC | 2559 |
| | LFRW2 | WYQQHPGKAPKLMTY | 2560 |
| | LFRW3 | GVPARFSGSKSDNTASLTISGLQAEDEADYYC | 2561 |
| | LFRW4 | FGGGTKLTVL | 2562 |
| S376-1721 | Heavy Chain | QVQLVQSGTEVREPGASVKVSCKASGYTFTGYYVHWVRQAPGQGLE | 2563 |
| (Spike) | | WMGWVNPGSGDTLYAQKFQGRFTLTRDMSITTAYMELSSLRSDDSA | |
| | | VYFCFRGYSYATFDYWGQGTLVTVSSASPTSPKVFPLSLCSTQPDGN | |
| | | VVIACLVQGFFPQEPLSVTWSESGQGVTARNF~ | |
| | Heavy Chain | QVQLVQSGTEVREPGASVKVSCKASGYTFTGYYVHWVRQAPGQGLE | 2564 |
| | Variable | WMGWVNPGSGDTLYAQKFQGRFTLTRDMSITTAYMELSSLRSDDSA | |
| | Region | VYFCFRGYSYATFDYWGQGTLVTVSS | |
| | HCDR1 | GYYVH | 2565 |
| | HCDR2 | WVNPGSGDTLYAQKFQG | 2566 |
| | HCDR3 | GYSYATFDY | 2567 |
| | HFRW1 | QVQLVQSGTEVREPGASVKVSCKASGYTFT | 2568 |
| | HFRW2 | WVRQAPGQGLEWMG | 2569 |
| | HFRW3 | RFTLTRDMSITTAYMELSSLRSDDSAVYFCFR | 2570 |
| | HFRW4 | WGQGTLVTVSS | 2571 |
| | Light Chain | QSVLTQPPSVSGAPGQRVTISCTGSSSNIGAGYDVHWYQQLPGTAPKL | 2572 |
| | | LIYGNSNRPSGVPDRFSGSKSGTSASLAITGLQAEDEADYYCQSYDSS | |
| | | LSGSFYVFGTGTKVTVLGQPKANPTVTLFPPSSEELQANKATLVCLIS | |
| | | DFYPGAVTVAWKADSSPVKAGVETTTPSKQSNNKYAASS~ | |
| | Light Chain | QSVLTQPPSVSGAPGQRVTISCTGSSSNIGAGYDVHWYQQLPGTAPKL | 2573 |
| | Variable | LIYGNSNRPSGVPDRFSGSKSGTSASLAITGLQAEDEADYYCQSYDSS | |
| | Region | LSGSFYVFGTGTKVTVL | |
| | LCDR1 | TGSSSNIGAGYDVH | 2574 |
| | LCDR2 | GNSNRPS | 2575 |
| | LCDR3 | QSYDSSLSGSFYV | 2576 |
| | LFRW1 | QSVLTQPPSVSGAPGQRVTISC | 2577 |
| | LFRW2 | WYQQLPGTAPKLLIY | 2578 |
| | LFRW3 | GVPDRFSGSKSGTSASLAITGLQAEDEADYYC | 2579 |
| | LFRW4 | FGTGTKVTVL | 2580 |
| S376-2486 | Heavy Chain | QVQLVESGGGVVQPGRSLRLSCAVSGFTFSSYAMHWVRQAPGKGLE | 2581 |
| (Spike) | | WVAVISYDGSNKYFADSVKGRFTISRDNSKNTLYLQMNSLRAEDTAV | |
| | | YYCARGRGNYFTYFDYWGQGTLVTVSSASTKGPSVFPLAPSSKSTSG | |
| | | GTAALGCLVKDYFPEPVTVSWNSGALTSGVHTFPAVLQSS~ | |
| | Heavy Chain | QVQLVESGGGVVQPGRSLRLSCAVSGFTFSSYAMHWVRQAPGKGLE | 2582 |
| | Variable | WVAVISYDGSNKYFADSVKGRFTISRDNSKNTLYLQMNSLRAEDTAV | |
| | Region | YYCARGRGNYFTYFDYWGQGTLVTVSS | |
| | HCDR1 | SYAMH | 2583 |
| | HCDR2 | VISYDGSNKYFADSVKG | 2584 |
| | HCDR3 | GRGNYFTYFDY | 2585 |
| | HFRW1 | QVQLVESGGGVVQPGRSLRLSCAVSGFTFS | 2586 |
| | HFRW2 | WVRQAPGKGLEWVA | 2587 |
| | HFRW3 | RFTISRDNSKNTLYLQMNSLRAEDTAVYYCAR | 2588 |
| | HFRW4 | WGQGTLVTVSS | 2589 |
| | Light Chain | EIVLTQSPGTLSLSPGERATLSCRASQSVSRNYLAWYQQKPGQAPRLL | 2590 |
| | | IYSASSRATGIPDRFSGSGSGTDFTLTISRLEPEDFAVYYCQQYGGSLTF | |
| | | GGGTKVEIKRTVAAPSVFIFPPSDEQLKSGTASVVCLLNNFYPREAKV | |
| | | QWKVDN~ | |
| | Light Chain | EIVLTQSPGTLSLSPGERATLSCRASQSVSRNYLAWYQQKPGQAPRLL | 2591 |
| | Variable | IYSASSRATGIPDRFSGSGSGTDFTLTISRLEPEDFAVYYCQQYGGSLTF | |
| | Region | GGGTKVEIK | |
| | LCDR1 | RASQSVSRNYLA | 2592 |
| | LCDR2 | SASSRAT | 2593 |
| | LCDR3 | QQYGGSLT | 2594 |
| | LFRW1 | EIVLTQSPGTLSLSPGERATLSC | 2595 |
| | LFRW2 | WYQQKPGQAPRLLIY | 2596 |
| | LFRW3 | GIPDRFSGSGSGTDFTLTISRLEPEDFAVYYC | 2597 |
| | LFRW4 | FGGGTKVEIK | 2598 |
| S376-780 | Heavy Chain | QVQLVESGGGVVQPGRSLRLSCAASGFTFSSYGMHWVRQAPGKGLE | 2599 |
| (Spike) | | WVAVISYDGSNKYYADSVKGRFTISRDNSKNTLYLQMNSLRAEDTA | |
| | | VYYCAKEGGSYSYYYYGMDVWGQGTTVTVSSGSASAPTLFPLVSCE | |
| | | NSPSDTSSV | |
| | Heavy Chain | QVQLVESGGGVVQPGRSLRLSCAASGFTFSSYGMHWVRQAPGKGLE | 2600 |
| | Variable | WVAVISYDGSNKYYADSVKGRFTISRDNSKNTLYLQMNSLRAEDTA | |
| | Region | VYYCAKEGGSYSYYYYGMDVWGQGTTVTVSS | |
| | HCDR1 | SYGMH | 2601 |
| | HCDR2 | VISYDGSNKYYADSVKG | 2602 |
| | HCDR3 | EGGSYSYYYYGMDV | 2603 |
| | HFRW1 | QVQLVESGGGVVQPGRSLRLSCAASGFTFS | 2604 |
| | HFRW2 | WVRQAPGKGLEWVA | 2605 |
| | HFRW3 | RFTISRDNSKNTLYLQMNSLRAEDTAVYYCAK | 2606 |
| | HFRW4 | WGQGTTVTVSS | 2607 |
| | Light Chain | DIQMTQSPSSLSASVGDRVTITCRASQGISNYLAWYQQKPGKVPKLLI | 2608 |
| | | YAASTLQSGVPSRFSGSGSGTDFTLTISSLQPEDVATYYCQKYNSAPR | |
| | | TFGPGTKVDIKRTVAAPSVFIFPPSDEQLKSGTASVVCLLNNFYPREAK | |
| | | VQWKVDN~ | |
| | Light Chain | DIQMTQSPSSLSASVGDRVTITCRASQGISNYLAWYQQKPGKVPKLLI | 2609 |
| | Variable | YAASTLQSGVPSRFSGSGSGTDFTLTISSLQPEDVATYYCQKYNSAPR | |
| | Region | TFGPGTKVDIK | |
| | LCDR1 | RASQGISNYLA | 2610 |
| | LCDR2 | AASTLQS | 2611 |
| | LCDR3 | QKYNSAPRT | 2612 |
| | LFRW1 | DIQMTQSPSSLSASVGDRVTITC | 2613 |
| | LFRW2 | WYQQKPGKVPKLLIY | 2614 |
| | LFRW3 | GVPSRFSGSGSGTDFTLTISSLQPEDVATYYC | 2615 |
| | LFRW4 | FGPGTKVDIK | 2616 |
| S469-373 | Heavy Chain | EVQLVESGGGLVQPGGSLRLSCVVSGFTFSRYWMSWVRQTPGKGLQ | 2617 |
| (NP) | | WVANIKQDDTNKFYEDSVKGRFTTSRDNAKNSLYLQMNSLRAEDTA | |
| | | VYYCARGGGSSSGLYFESWGQGTLVIVSSGSASAPTLFPLVSCENSPS | |
| | | DTSSV | |
| | Heavy Chain | EVQLVESGGGLVQPGGSLRLSCVVSGFTFSRYWMSWVRQTPGKGLQ | 2618 |
| | Variable | WVANIKQDDTNKFYEDSVKGRFTTSRDNAKNSLYLQMNSLRAEDTA | |
| | Region | VYYCARGGGSSSGLYFESWGQGTLVIVSS | |
| | HCDR1 | RYWMS | 2619 |
| | HCDR2 | NIKQDDTNKFYEDSVKG | 2620 |
| | HCDR3 | GGGSSSGLYFES | 2621 |
| | HFRW1 | EVQLVESGGGLVQPGGSLRLSCVVSGFTFS | 2622 |
| | HFRW2 | WVRQTPGKGLQWVA | 2623 |
| | HFRW3 | RFTTSRDNAKNSLYLQMNSLRAEDTAVYYCAR | 2624 |
| | HFRW4 | WGQGTLVIVSS | 2625 |
| | Light Chain | EVQLVESGGGLVQPGGSLRLSCVVSGFTFSRYWMSWVRQTPGKGLQ | 2626 |
| | | WVANIKQDDTNKFYEDSVKGRFTTSRDNAKNSLYLQMNSLRAEDTA | |
| | | VYYCARGGGSSSGLYFESWGQGTLVIVSSGSASAPTLFPLVSCENSPS | |
| | | DTSSV | |
| | Light Chain | EVQLVESGGGLVQPGGSLRLSCVVSGFTFSRYWMSWVRQTPGKGLQ | 2627 |
| | Variable | WVANIKQDDTNKFYEDSVKGRFTTSRDNAKNSLYLQMNSLRAEDTA | |
| | Region | VYYCARGGGSSSGLYFESWGQGTLVIVSS | |
| | LCDR1 | RYWMS | 2628 |
| | LCDR2 | NIKQDDTNKFYEDSVKG | 2629 |
| | LCDR3 | GGGSSSGLYFES | 2630 |
| | LFRW1 | EVQLVESGGGLVQPGGSLRLSCVVSGFTFS | 2631 |
| | LFRW2 | WVRQTPGKGLQWVA | 2632 |
| | LFRW3 | RFTTSRDNAKNSLYLQMNSLRAEDTAVYYCAR | 2633 |
| | LFRW4 | WGQGTLVIVSS | 2634 |
| S48-144 | Heavy Chain | EVQLVESGGDLVQPGRSLRLSCTASAFNFGDYAMSWVRQAPGKGLE | 2635 |
| (Spike) | | WVGFIRSKGYGGTTEYAASVKGRFTISRDDSNRIAYLQMNSLKSEDT | |
| | | AVYYCSRGYQLPNLWGQGTLVTVSSASPTSPKVFPLSLCSTQPDGNV | |
| | | VIACLVQGFFPQEPLSVTWSESGQGVTARNF~ | |
| | Heavy Chain | EVQLVESGGDLVQPGRSLRLSCTASAFNFGDYAMSWVRQAPGKGLE | 2636 |
| | Variable | WVGFIRSKGYGGTTEYAASVKGRFTISRDDSNRIAYLQMNSLKSEDT | |
| | Region | AVYYCSRGYQLPNLWGQGTLVTVSS | |
| | HCDR1 | DYAMS | 2637 |
| | HCDR2 | FIRSKGYGGTTEYAASVKG | 2638 |
| | HCDR3 | GYQLPNL | 2639 |
| | HFRW1 | EVQLVESGGDLVQPGRSLRLSCTASAFNFG | 2640 |
| | HFRW2 | WVRQAPGKGLEWVG | 2641 |
| | HFRW3 | RFTISRDDSNRIAYLQMNSLKSEDTAVYYCSR | 2642 |
| | HFRW4 | WGQGTLVTVSS | 2643 |
| | Light Chain | DIQMTQSPSSLSASVGDRVTITCRASQSISTFLNWYQQKPGKAPSLLIY | 2644 |
| | | AASSLQSGVPSRFSGSESGTDFTLTISSLQPEDFATYYCQQSYSTPLTFG | |
| | | GGTKVEIKRTVAAPSVFIFPPSDEQLKSGTASVVCLLNNFYPREAKVQ | |
| | | WKVDN~ | |
| | Light Chain | DIQMTQSPSSLSASVGDRVTITCRASQSISTFLNWYQQKPGKAPSLLIY | 2645 |
| | Variable | AASSLQSGVPSRFSGSESGTDFTLTISSLQPEDFATYYCQQSYSTPLTFG | |
| | Region | GGTKVEIK | |
| | LCDR1 | RASQSISTFLN | 2646 |
| | LCDR2 | AASSLQS | 2647 |
| | LCDR3 | QQSYSTPLT | 2648 |
| | LFRW1 | DIQMTQSPSSLSASVGDRVTITC | 2649 |
| | LFRW2 | WYQQKPGKAPSLLIY | 2650 |
| | LFRW3 | GVPSRFSGSESGTDFTLTISSLQPEDFATYYC | 2651 |
| | LFRW4 | FGGGTKVEIK | 2652 |
| S564-128 | Heavy Chain | EVHLVESGGGWVQPGGSLRLSCAASGFTLSTYWMSWVRQTPGEGLQ | 2653 |
| (NP) | | WVANIKQDGSSKYYVDSVKGRFTISRDNAKNSVYLQMNSLRGEDTA | |
| | | VYYCARGDGSNSGIYFDSWGQGTLVTVSSASTKGPSVFPLAPCSRSTS | |
| | | ESTAALGCLVKDYFPEPVTVSWNSGALTSGVHTFPAVLQSSG | |
| | Heavy Chain | EVHLVESGGGWVQPGGSLRLSCAASGFTLSTYWMSWVRQTPGEGLQ | 2654 |
| | Variable | WVANIKQDGSSKYYVDSVKGRFTISRDNAKNSVYLQMNSLRGEDTA | |
| | Region | VYYCARGDGSNSGIYFDSWGQGTLVTVSS | |
| | HCDR1 | TYWMS | 2655 |
| | HCDR2 | NIKQDGSSKYYVDSVKG | 2656 |
| | HCDR3 | GDGSNSGIYFDS | 2657 |
| | HFRW1 | EVHLVESGGGWVQPGGSLRLSCAASGFTLS | 2658 |
| | HFRW2 | WVRQTPGEGLQWVA | 2659 |
| | HFRW3 | RFTISRDNAKNSVYLQMNSLRGEDTAVYYCAR | 2660 |
| | HFRW4 | WGQGTLVTVSS | 2661 |
| | Light Chain | EIVMTQSPATLSVSPGERATLSCRASQSISSKLAWYQQKPGQAPRLLIY | 2662 |
| | | GASTRATGIPARFSGSGSGTEFTLTISSMQSEDFAVYYCQQYNYWYTF | |
| | | GQGTKLEIKRTVAAPSVFIFPPSDEQLKSGTASVVCLLNNFYPREAKV | |
| | | QWKVDNALQSGNSQESVTEQDSKDSTYSLSSTLTLSKADYE~ | |
| | Light Chain | EIVMTQSPATLSVSPGERATLSCRASQSISSKLAWYQQKPGQAPRLLIY | 2663 |
| | Variable | GASTRATGIPARFSGSGSGTEFTLTISSMQSEDFAVYYCQQYNYWYTF | |
| | Region | GQGTKLEIK | |
| | LCDR1 | RASQSISSKLA | 2664 |
| | LCDR2 | GASTRAT | 2665 |
| | LCDR3 | QQYNYWYT | 2666 |
| | LFRW1 | EIVMTQSPATLSVSPGERATLSC | 2667 |
| | LFRW2 | WYQQKPGQAPRLLIY | 2668 |
| | LFRW3 | GIPARFSGSGSGTEFTLTISSMQSEDFAVYYC | 2669 |
| | LFRW4 | FGQGTKLEIK | 2670 |
| S92-110 | Heavy Chain | EVQLVESGGGLVQPGGSLRLSCAASGFTFSSYEMNWVRQAPGKGLE | 2671 |
| (NP) | | WVSYISSSGSTIYYADSVKGRFTISRDNAKNSLYLQMNSLRAEDTAVY | |
| | | YCARDRRGDYGRYYYGMDVWGQGTTVTVSSGSASAPTLFPLVSCEN | |
| | | SPSDTSSV | |
| | Heavy Chain | EVQLVESGGGLVQPGGSLRLSCAASGFTFSSYEMNWVRQAPGKGLE | 2672 |
| | Variable | WVSYISSSGSTIYYADSVKGRFTISRDNAKNSLYLQMNSLRAEDTAVY | |
| | Region | YCARDRRGDYGRYYYGMDVWGQGTTVTVSS | |
| | HCDR1 | SYEMN | 2673 |
| | HCDR2 | YISSSGSTIYYADSVKG | 2674 |
| | HCDR3 | DRRGDYGRYYYGMDV | 2675 |
| | HFRW1 | EVQLVESGGGLVQPGGSLRLSCAASGFTFS | 2676 |
| | HFRW2 | WVRQAPGKGLEWVS | 2677 |
| | HFRW3 | RFTISRDNAKNSLYLQMNSLRAEDTAVYYCAR | 2678 |
| | HFRW4 | WGQGTTVTVSS | 2679 |
| | Light Chain | SSELTQDPAVSVALGQTVRITCQGDSLRSYYASWYQQKPGQAPVLVI | 2680 |
| | | YGKNNRPSGIPDRFSGSSSGNTASLTITGAQAEDEADYYCNSRDSSGN | |
| | | RVFGGGTKLTVLGQPKAAPSVTLFPPSSEELQANKATLVCLISDFYPG | |
| | | AVTVAWKADSSPVKAGVETTTPSKQSNNKYAASS~ | |
| | Light Chain | SSELTQDPAVSVALGQTVRITCQGDSLRSYYASWYQQKPGQAPVLVI | 2681 |
| | Variable | YGKNNRPSGIPDRFSGSSSGNTASLTITGAQAEDEADYYCNSRDSSGN | |
| | Region | RVFGGGTKLTVL | |
| | LCDR1 | QGDSLRSYYAS | 2682 |
| | LCDR2 | GKNNRPS | 2683 |
| | LCDR3 | NSRDSSGNRV | 2684 |
| | LFRW1 | SSELTQDPAVSVALGQTVRITC | 2685 |
| | LFRW2 | WYQQKPGQAPVLVIY | 2686 |
| | LFRW3 | GIPDRFSGSSSGNTASLTITGAQAEDEADYYC | 2687 |
| | LFRW4 | FGGGTKLTVL | 2688 |
| S92-2329 | Heavy Chain | EVQLVESGGGLVKPGGSLRLSCAASGFTFSSYSMNWVRQAPGKGLE | 2689 |
| (Spike) | | WVSSISSSGTYIYYADSVKGRFTISRDNAKNSLYLQMNSLRVEDTAVY | |
| | | YCAQSIAARLDWFDPWGQGTLVTVSSGSASAPTLFPLVSCENSPSDTS | |
| | | SV | |
| | Heavy Chain | EVQLVESGGGLVKPGGSLRLSCAASGFTFSSYSMNWVRQAPGKGLE | 2690 |
| | Variable | WVSSISSSGTYIYYADSVKGRFTISRDNAKNSLYLQMNSLRVEDTAVY | |
| | Region | YCAQSIAARLDWFDPWGQGTLVTVSS | |
| | HCDR1 | SYSMN | 2691 |
| | HCDR2 | SISSSGTYIYYADSVKG | 2692 |
| | HCDR3 | SIAARLDWFDP | 2693 |
| | HFRW1 | EVQLVESGGGLVKPGGSLRLSCAASGFTFS | 2694 |
| | HFRW2 | WVRQAPGKGLEWVS | 2695 |
| | HFRW3 | RFTISRDNAKNSLYLQMNSLRVEDTAVYYCAQ | 2696 |
| | HFRW4 | WGQGTLVTVSS | 2697 |
| | Light Chain | EIVLTQSPATLSLSPGERATLSCRASQSVSSYLAWYQQKPGQAPRLLIY | 2698 |
| | | DAFNRATGIPARFSGSGSGTDFTLTISSLEPEDFAVYYCQQRSNWPRTF | |
| | | GGGTKVEIKRTVAAPSVFIFPPSDEQLKSGTASVVCLLNNFYPREAKV | |
| | | QWKVDN~ | |
| | Light Chain | EIVLTQSPATLSLSPGERATLSCRASQSVSSYLAWYQQKPGQAPRLLIY | 2699 |
| | Variable | DAFNRATGIPARFSGSGSGTDFTLTISSLEPEDFAVYYCQQRSNWPRTF | |
| | Region | GGGTKVEIK | |
| | LCDR1 | RASQSVSSYLA | 2700 |
| | LCDR2 | DAFNRAT | 2701 |
| | LCDR3 | QQRSNWPRT | 2702 |
| | LFRW1 | EIVLTQSPATLSLSPGERATLSC | 2703 |
| | LFRW2 | WYQQKPGQAPRLLIY | 2704 |
| | LFRW3 | GIPARFSGSGSGTDFTLTISSLEPEDFAVYYC | 2705 |
| | LFRW4 | FGGGTKVEIK | 2706 |

TABLE 2
Nucleic Acid Sequences

| | | SEQ | | SEQ |
|---|---|---|---|---|
| | | ID | | ID |
| Clone | HC Sequence | NO: | LC Sequence | NO: |
| S20-15 | CAGGTGCAGCTGCAGGAGTCGGGCC | 1621 | TCCTATGTGCTGACTCAGCCACCCT | 1711 |
| | CAGGACTGGTGAGGCCTTCGGAGAC | | CGGTGTCAGTGGCCCCAGGACAGA | |
| | CCTGTCCCTCACCTGCACTGTCTCTG | | CGGCCAGGATTACCTGTGGGGGAA | |
| | GTGGCTCCATCAGTAGTCACTACTG | | ACAACATTGGAAGTAAAAGTGTGC | |
| | GAGCTGGATCCGGCAGCCCCCCGGG | | ACTGGTACCAGCAGAAGCCAGGCC | |
| | AAGGGACTGGAGTGGATTGGGTATA | | AGGCCCCTGTGCTGGTCGTCTATGA | |
| | TCTATTATAGTGGGAGCACCAATTA | | TGATAGCGACCGGCCCTCAGGGAT | |
| | CAACCCCTCCCTCAAGAGTCGAGTC | | CCCTGAGCGATTCTCTGGCTCCAAC | |
| | ACCATATCAGTAGACACGTCCAAGA | | TCTGGGAACACGGCCACCCTGACC | |
| | ACCAGTTCTCCCTGAAACTTATCTCT | | ATCAGCAGGGTCGAAGCCGGGGAT | |
| | GTGACCGCTGCGGACACGGCCGTGT | | GAGGCCGACTATTACTGTCAGGTG | |
| | ATTACTGTGCGAGAGCCGGGGGCGT | | TGGGATAGTAGTAGTGAGCATTAT | |
| | TTTTGGAGTGGTTCTGGACTTTGACC | | GTCTTCGGAACTGGGACCAAGGTC | |
| | ACTGGGGCCGGGGAACCCTGGTCAC | | ACCGTCCTAGGTCAGCCCAAGGCC | |
| | CGTCTCCTCAGCCTCCACCAAGGGC | | AACCCCACTGTCACTCTGTTCCCGC | |
| | CCATCGGTCTTCCCCCTGGCACCCTC | | CCTCCTCTGAGGAGCTCCAAGCCA | |
| | CTCCAAGAGCACCTCTGGGGGCACA | | ACAAGGCCACACTAGTGTGTCTGA | |
| | GCGGCCCTGGGCTGCCTGGTCAAGG | | TCAGTGACTTCTACCCGGGAGCTGT | |
| | ACTACTTCCCCGAACCGGTGACGGT | | GACAGTGGCCTGGAAGGCAGATGG | |
| | GTCGTGGAACTCAGGCGCCCTGACC | | CAGCCCCGTCAAGGCGGGAGTGGA | |
| | AGCGGCGTGCACACCTTCCCGGCTG | | GACCACCAAACCCTCCAAACAGAG | |
| | TCCTACAGTCCTCAGGA | | CAACAACAAGTACGCGGCCAGCAG | |
| | | | CTA | |
| S20-22 | CAGGTGCAGCTGCAGGAGTCGGGCC | 1622 | GACATCGTGATGACCCAGTCTCCA | 1712 |
| | CAGGACTGGTGAAGCCTTCGGAGAC | | GACTCCCTGGCTGTGTCTCTGGGCG | |
| | CCTGTCCCTCACCTGCACTGTCTCTG | | AGAGGGCCACCATCAACTGCAAGT | |
| | GTGGCTCCATCAGTAGTTTCTACTG | | CCAGCCAGACTGTTTTATACAGCTC | |
| | GGGCTGGATCCGGCAGCCCGCCGGG | | CAACAATAAGAACTACTTAGCTTG | |
| | AAGGGACTGGAGTGGATTGGGCGTT | | GTACCAGCAGAAACCAGGACAGCC | |
| | TCCATACTAGTGGGAGCACCAACTA | | TCCTAAGTTGCTCATTTACTGGGCA | |
| | CAACCCCTCCTTCAAGAGTCGAGTC | | TCTACCCGGGAATCCGGGGTCCCT | |
| | ACCATGTCAGTAGACACGTCCAAGA | | GACCGATTCAGTGGCAGCGGGTCT | |
| | ACCAGTTCTCCCTGAAGCTGACCTC | | GGGACAGATTTCACTCTCACCATC | |
| | TGTGACCGCCGCGGACACGGCCGTG | | AGCAGCCTGCAGGCTGGAGATGTG | |
| | TATTACTGTGCGAGCGGCCGGGGCA | | GCAGTTTATTACTGTCAGCAATATT | |
| | GCAGCTGGTACGTAGGCTGGTTCTT | | ATAATACTCCGGACACTTTCGGCG | |
| | CGATCTCTGGGGCCGTGGCACCCTG | | GAGGGACCAAGGTGGAGATCAATC | |
| | GTCACTGTCTCCTCAGCCTCCACCA | | GAACTGTGGCTGCACCATCTGTCTT | |
| | AGGGCCCATCGGTCTTCCCCCTGGC | | CATCTTCCCGCCATCTGATGAGCAG | |
| | ACCCTCCTCCAAGAGCACCTCTGGG | | TTGAAATCTGGAACTGCCTCTGTTG | |
| | GGCACAGCAGCCCTGGGCTGCCTGG | | TGTGCCTGCTGAATAACTTCTATCC | |
| | TCAAGGACTACTTCCCCGAACCGGT | | CAGAGAGGCCAAAGTACAGTGGAA | |
| | GACGGTGTCGTGGAACTCAGGCGCC | | GGTGGATAACGC | |
| | CTGACCAGCGGCGTGCACACCTTCC | | | |
| | CGGCTGTCCTACAGTCCTCAGGA | | | |
| S20-31 | CAGGTCCAACTCATACAGTCAGGGG | 1623 | GAAATTGTGTTGACGCAGTCCCCA | 1713 |
| | CTGAGGTGAAGAAGCCTGGGGCCTC | | GGCACCCTGTCTTTGTCTCCAGGGG | |
| | AGTGAAGGTCTCCTGCACGGCCTCC | | AAAGAGCCACCCTCTCCTGCAGGG | |
| | GGATACTCCCTCAATGAGTTGCCCA | | CCAGTCAGGATATTACCAACAACT | |
| | TACAGTGGGTGCGGCAGGCTCCTGG | | TCTTAGCCTGGTACCAGCAGAAAG | |
| | TAAAGGGCTTGAGTGGATGGGAGA | | CCGGCCAGGCTCCCAAACTCTTCAT | |
| | ATTTGATCCCGAAGATGGTGAAACA | | CTATGGTGCATCCAGGAGGGCCCC | |
| | ATCTACGCAGAGAAATTCCAGGGCA | | TGGCATCCCACACAGGTTCAGTGG | |
| | GAGTCACCCTGACCGAGGAAACATC | | CAGTGGGTCTGGGACAGACTTCAC | |
| | TACAAACACAGCCTACATGGAGTTG | | TCTCACCATCAGCAGCCTGGAGCC | |
| | AGCAGCCTGAAATCTGAGGACACG | | TGAAGATTTTGCAGTATATTACTGT | |
| | GCCGCGTATTTTTGTTCAACCGGCTC | | CAGCAGTACGGTCCCTCTCCGACG | |
| | GACTATTGGCGTCGTCATTTATGCTT | | TTCGGCCAAGGGACCAAGGTGGAA | |
| | TTGCTATCTGGGGCCAAGGGACAAT | | ATCAAACGAACTGTGGCTGCACCA | |
| | GGTCACCGTCTCTTCAGCTTCCACC | | TCTGTCTTCATCTTCCCGCCATCTG | |
| | AAGGGCCCATCGGTCTTCCCCCTGG | | ATGAGCAGTTGAAATCTGGAACTG | |
| | CGCCCTGCTCCAGGAGCACCTCCGA | | CCTCTGTTGTGTGCCTGCTGAATAA | |
| | GAGCACAGCCGCCCTGGGCTGCCTG | | CTTCTATCCCAGAGAGGCCAAAGT | |
| | GTCAAGGACTACTTCCCCGAACCGG | | ACAGTGGAAGGTGGATAACGCCCT | |
| | TGACGGTGTCGTGGAACTCAGGCGC | | CCAATCGGGTAACTCCCAGGAGAG | |
| | CCTGACCAGCGGCGTGCACACCTTC | | TGTCACAGAGCAGGACAGCAAGGA | |
| | CCGGCTGTCCTACAGTCCTCAGGA | | CAGCACCTACAGCCTCAGCAGCAC | |
| | | | CCTGACGCTGAGCAAAGCAGACTA | |
| | | | CGAGAA | |
| S20-40 | CAGGTGCAGCTGCAGGAGTCGGGCC | 1624 | CAGTCTGCCCTGACTCAGCCTGCCT | 1714 |
| | CAGGACTGGTGAAGCCTTCGGAGAC | | CCGTGTCTGGGTCTCCTGGACAGTC | |
| | CCTGTCCCTCACCTGCACTGTCTCTG | | GATCACCATCTCCTGCACTGGAAC | |
| | GTGGCTCCATCAGTAGTTACTACTG | | CAGCAGTGACGTTGGTGGTTATAA | |
| | GAGCTGGATCCGGCAGCCCGCCGGG | | CTATGTCTCCTGGTACCAACAGCAC | |
| | AAGGGACTGGAGTGGATTGGGCGT | | CCAGGCAAAGCCCCCAAACTCATG | |
| | ATCTATACCAGTGGGAGCACCAACT | | ATTTATGATGTCAGTAATCGGCCCT | |
| | ACAACCCCTCCCTCAAGAGTCGAGT | | CAGGGGTTTCTAATCGCTTCTCTGG | |
| | CACCATGTCAGTAGACACGTCCAAG | | CTCCAAGTCTGGCAACACGGCCTC | |
| | AACCAGTTCTCCCTGAAGCTGAGCT | | CCTGACCATCTCTGGGCTCCAGGCT | |
| | CTGTGACCGCCGCGGACACGGCCGT | | GAGGACGAGGCTGATTATTACTGC | |
| | GTATTACTGTGCGAGAGGGGGCAGT | | AGCTCATATACAAGCAGCAGCACT | |
| | GGCTGGCGCTTTGACTACTGGGGCC | | CTCGGAGTGTTCGGCGGAGGGACC | |
| | AGGGAACCCTGGTCACCGTCTCCTC | | AAGCTGACCGTCCTAGGTCAGCCC | |
| | AGGGAGTGCATCCGCCCCAACCCTT | | AAGGCTGCCCCCTCGGTCACTCTGT | |
| | TTCCCCCTCGTCTCCTGTGAGAATTC | | TCCCACCCTCCTCTGAGGAGCTTCA | |
| | CCCGTCGGATACGAGCAGCGTG | | AGCCAACAAGGCCACACTGGTGTG | |
| | | | TCTCATAAGTGACTTCTACCCGGGA | |
| | | | GCCGTGACAGTGGCCTGGAAGGCA | |
| | | | GATAGCAGCCCCGTCAAGGCGGGA | |
| | | | GTGGAGACCACCACACCCTCCAAA | |
| | | | CAAAGCAACAACAAGTACGCGGCC | |
| | | | AGCAGCTA | |
| S20-58 | CAGGTGCAGCTGCAGGAGTCGGGCC | 1625 | GATATTGTGATGACCCAGACTCCA | 1715 |
| | CAGGACTGGTGAAGCCTTCACAGAC | | CTCTCCTCACCTGTCACCCTTGGAC | |
| | CCTGTCCCTCACCTGCACTGTCTCTG | | AGCCGGCCTCCATCTCCTGCAGGTC | |
| | GTGGCTCCATCAACAGTGGTGATTA | | TAGTCAAAGCCTCGTACACAGTGA | |
| | CTACTGGAGCTGGATCCGCCAGCCC | | TGGAGACACCTACTTGAGTTGGCTT | |
| | CCAGGGAAGGGCCTGGAGTGGATT | | CAGCAGAGGCCAGGCCAGCCTCCA | |
| | GGGTACATCTATTTCAGTGGGAGCA | | AGACTCCTAATTTACAAGATTTCTA | |
| | CCTACTACAACCCGTCCCTCAAGAG | | ACCGGTTCTCTGGGGTCCCAGACA | |
| | TCGAGTTACCATATCACTAGACAGG | | GATTCAGTGGCAGTGGGGCAGGGA | |
| | TCCAAGAACCAGTTCTCCCTGAAGC | | CAGATTTCACACTGAAAATCAGCA | |
| | TGAGCTCTGTGACTGCCGCAGACAC | | GGGTGGAAGCTGAGGATGTCGGGG | |
| | GGCCGTGTATTACTGTGCCAGAGAG | | TTTATTACTGCATGCAAGCTACACA | |
| | GAAAGTATGATTACGCTTGGGGGAG | | ATTTCCTCTCACTTTCGGCGGAGGG | |
| | TTATCGTCGACTGGGGCCAGGGAAC | | ACCAAGGTGGAGATCAAACGAACT | |
| | CCTGGTCACCGTCTCCTCAGCCTCC | | GTGGCTGCACCATCTGTCTTCATCT | |
| | ACCAAGGGCCCATCGGTCTTCCCCC | | TCCCGCCATCTGATGAGCAGTTGA | |
| | TGGCACCCTCCTCCAAGAGCACCTC | | AATCTGGAACTGCCTCTGTTGTGTG | |
| | TGGGGGCACAGCAGCCCTGGGCTGC | | CCTGCTGAATAACTTCTATCCCAGA | |
| | CTGGTCAAGGACTACTTCCCCGAAC | | GAGGCCAAAGTACAGTGGAAGGTG | |
| | CGGTGACGGTGTCGTGGAACTCAGG | | GATAACGCCCTCCAATCGGGTAAC | |
| | CGCCCTGACCAGCGGCGTGCACACC | | TCCCAGGAGAGTGTCACAGAGCAG | |
| | TTCCCGGCTGTCCTACAGTCCTCAG | | GACAGCAAGGACAGCACCTACAGC | |
| | GA | | CTCAGCAGCACCCTGACGCTGAGC | |
| | | | AAAGCAGACTACGAGAA | |
| S20-74 | CAGGTGCAGCTGCAGGAGTCGGGCC | 1626 | CAGTCTGCCCTGACTCAGCCTCCCT | 1716 |
| | CAGGACTGGTGAAGCCTTCGGAGAC | | CCGCGTCCGGGTCTCCTGGACAGT | |
| | CCTGTCCCTCACCTGCACTGTCTCTG | | CAGTCACCATCTCCTGCACTGGAA | |
| | GTGGCTCCATCAGTAGTCACTACTG | | CCAGCAGTGACGTTGGTGGTTATA | |
| | GAGCTGGATCCGGCAGCCCCCAGGG | | ACTATGTCTCCTGGTACCAACAGC | |
| | AAGGGACTGGAGCAGATTGGGTAT | | ACCCAGGCAAAGCCCCCAAACTCA | |
| | ATGTATTACAGTGGGAGCACCAACT | | TGATTTATGAGGTCAGTAAGCGGC | |
| | ACAACCCCTCCCTCAAGAGTCGAGT | | CCTCAGGGGTCCCTGATCGCTACTC | |
| | CATCATATCAGTAGACACGTCCAAG | | TGGCTCCAAGTCTGGCAACACGGC | |
| | AACCAGTTCTCCCTGAAGTTGAGCT | | CTCCCTGACCGTCTCTGGGCTCCAG | |
| | CTGTGACCGCTGCGGACACGGCCGT | | GCTGAGGATGAGGCTGATTATTAC | |
| | GTATTACTGTGCGGGTCGTGACCAG | | TGCAGCTCATATGCAGGCAGCAGC | |
| | CTGTTATACGGGGCCGATGGTTTTG | | AATCATGTGATATTCGGCGGAGGG | |
| | ATATCTGGGGCCAAGGGACAATGGT | | ACCAAGCTGACCGTCCTAGGTCAG | |
| | CACCGTCTCTTCAGCCTCCACCAAG | | CCCAAGGCTGCCCCCTCGGTCACTC | |
| | GGCCCATCGGTCTTCCCCCTGGCAC | | TGTTCCCGCCCTCCTCTGAGGAGCT | |
| | CCTCCTCCAAGAGCACCTCTGGGGG | | TCAAGCCAACAAGGCCACACTGGT | |
| | CACAGCGGCCCTGGGCTGCCTGGTC | | GTGTCTCATAAGTGACTTCTACCCG | |
| | AAGGACTACTTCCCCGAACCGGTGA | | GGAGCCGTGACAGTGGCCTGGAAG | |
| | CGGTGTCGTGGAACTCAGGCGCCCT | | GCAGATAGCAGCCCCGTCAAGGCG | |
| | GACCAGCGGCGTGCACACCTTCCCG | | GGAGTGGAGACCACCACACCCTCC | |
| | GCTGTCCTACAGTCCTCAGGA | | AAACAAAGCAACAACAAGTACGCG | |
| | | | GCCAGCAGCTA | |
| S20-86 | GAAGTGCAGCTGGTGGAGTCTGGGG | 1627 | CAGTCTGCCCTGACTCAGCCTGCCT | 1717 |
| | GAGGCTTGGTACAGCCTGGCAGGTC | | CCGTGTCTGGGTCTCCTGGACAGTC | |
| | CCTGAGACTCTCCTGTGCAGCCTCT | | GATCACCATCTCCTGCACTGGAAC | |
| | GGATTCACCTTTGGTGACTATGCCA | | CAGCAGTGACGTTGGTGGTTATAA | |
| | TGTACTGGGTCCGGCAACCTCCAGG | | CTATGTCTCCTGGTACCAACAACAC | |
| | GAAGGGCCTGGAGTGGGTCTCAGGT | | CCAGGCAAAGCCCCCAAACTCATG | |
| | ATTAGTTGGAATAGAGGTACTATAG | | ATTTATGATGTCAGTAATCGGCCCT | |
| | GCTATGCGGACTCTGTGAAGGGCCG | | CAGGGGTTTCTAATCGCTTCTCTGG | |
| | ATTCACCATCTCCAGAGACAACGCC | | CTCCAAGTCTGGCAACACGGCCTC | |
| | AAGAACTCCCTGTATCTGCAAATGA | | CCTGACCATCTCTGGGCTCCAGGCT | |
| | ACAGTCTGACACCTGAGGACACGGC | | GAGGACGAGGCTGATTATTACTGC | |
| | CTTGTATTACTGTGCAAAAGATATG | | AGCTCATATACAAGCAGCAGCACT | |
| | CTACCAGCTAGTAGGTTCTTCTACT | | CTCGGCGTCTTCGGAACTGGGACC | |
| | ACATGGACGTCTGGGGCAAAGGGA | | AAGGTCACCGTCCTAGGTCAGCCC | |
| | CCACGGTCATCGTCTCCTCAGCCTC | | AAGGCCAACCCCACTGTCACTCTG | |
| | CACCAAGGGCCCATCGGTCTTCCCC | | TTCCCGCCCTCCTCTGAGGAGCTCC | |
| | CTGGCACCCTCCTCCAAGAGCACCT | | AAGCCAACAAGGCCACACTAGTGT | |
| | CTGGGGGCACAGCAGCCCTGGGCTG | | GTCTGATCAGTGACTTCTACCCGGG | |
| | CCTGGTCAAGGACTACTTCCCCGAA | | AGCTGTGACAGTGGCCTGGAAGGC | |
| | CCGGTGACGGTGTCGTGGAACTCAG | | AGATGGCAGCCCCGTCAAGGCGGG | |
| | GCGCCCTGACCAGCGGCGTGCACAC | | AGTGGAGACCACCAAACCCTCCAA | |
| | CTTCCCGGCTGTCCTACAGTCCTCA | | ACAGAGCAACAACAAGTACGCGGC | |
| | GGA | | CAGCAGCTA | |
| S24-68 | CAGGTGCAGCTGCAGGAGTCGGGCC | 1628 | CAGTCTGTGCTGACTCAGCCACCCT | 1718 |
| | CAGGACTGGTGAAGCCTTCGGAGAC | | CAGCGTCTGGGACCCCCGGGCAGA | |
| | CCTGTCCCTCACCTGCACTGTCTCTG | | GGGTCACCATCTCTTGTTCTGGAAG | |
| | GTGGCTCCATCACTAGTTACTACTG | | CAGCTCCAACATCGGAGGTAATCC | |
| | GAGCTGGATCCGGCAGCCCCCAGGG | | TGTAAACTGGTACCAGCAGCTCCC | |
| | AAGGGACTGGAGTGGATTGAATATA | | AGGAACGGCCCCCAAACTCCTCAT | |
| | TCCATTACAGTGGGAGCACCAACTA | | CTATAGTAATAATCAGCGGCCCTC | |
| | CAACCCCTCCCTCAAGAGTCGAGTC | | AGGGGTCCCTGACCGATTCTCTGG | |
| | ACCATATCAGTAGACACGTCCAAGA | | CTCCAAGTCTGGCACCTCAGCCTCC | |
| | ACCAGTTCTCCCTGAAGCTGAGCTC | | CTGGCCATCAGTGGGCTCCAGTCT | |
| | TGTGACCGCTGCGGACACGGCCGTG | | GAGGATGAGGCTGATTATTACTGT | |
| | TATTACTGTGCGAGATTGCTCAAGT | | GCAGCATGGGATGACAGCCTGAAG | |
| | ATAGCAGGGGGGGGTGCTACTTTGA | | GGTCCGGTATTCGGCGGAGGGACC | |
| | CCACTGGGGCCAGGGAACCCTGGTC | | AAGCTGACCGTCCTAGGTCAGCCC | |
| | ACCGTCTCCTCAGCCTCCACCAAGG | | AAGGCTGCCCCCTCGGTCACTCTGT | |
| | GCCCATCGGTCTTCCCCCTGGCACC | | TCCCGCCCTCCTCTGAGGAGCTTCA | |
| | CTCCTCCAAGAGCACCTCTGGGGGC | | AGCCAACAAGGCCACACTGGTGTG | |
| | ACAGCGGCCCTGGGCTGCCTGGTCA | | TCTCATAAGTGACTTCTACCCGGGA | |
| | AGGACTACTTCCCCGAACCGGTGAC | | GCCGTGACAGTGGCCTGGAAGGCA | |
| | GGTGTCGTGGAACTCAGGCGCCCTG | | GATAGCAGCCCCGTCAAGGCGGGA | |
| | ACCAGCGGCGTGCACACCTTCCCGG | | GTGGAGACCACCACACCCTCCAAA | |
| | CTGTCCTACAGTCCTCAGGA | | CAAAGCAACAACAAGTACGCGGCC | |
| | | | AGCAGCTACCTGAGCCTGACGCCT | |
| | | | GAGCAGTGGAAGTCCCACA | |
| S24-105 | GAGGTGCAGCTGGTGGAGTCTGGGG | 1629 | GAAATTGTGTTGACGCAGTCTCCA | 1719 |
| | GAGGCTTGGTACAGCCGGGGGGGTC | | GGCACCCTGTCTTTGTCTCCAGGGG | |
| | CCTGAGACTCTCCTGTGCAGCCTCT | | AAAGAGCCACCCTCTCCTGCAGGG | |
| | GGATTCACCCTCAGCAGCTATAGCA | | CCAGTCAGAGTGTTAGCAGCGGTT | |
| | TGAACTGGGTCCGCCAGGCTCCAGG | | ACTTAGCCTGGTACCAGCAAAAAC | |
| | GAAGGGGCTGGAGTGGGTTTCATAC | | CTGGCCAGGCTCCCAGGCTCCTCAT | |
| | ATTAGTAGTAGTAGTAGCACCATAT | | CTTTGGTGCATCCAGCAGGGCCAC | |
| | ACTACGCAGACTCTGTGAAGGGCCG | | TGGCATCCCAGACAGGTTCAGTGG | |
| | ATTCACCATCTCCAAAGACAACGCC | | CAGTGGGTCTGGGACAGACTTCAC | |
| | AAGAACTCACTGTATCTGCAAATGA | | TCTCACCATCAACAGACTGGAGCC | |
| | ACAGCCTGAGAGCCGAGGACACGG | | TGAAGATTTTGCAGTGTATTACTGT | |
| | CTGTCTATTACTGTGCGGTCGGACG | | CAGCAGTATGGTAGCTCACGGACG | |
| | GGGATACTTTGTCTACTGGGGCCAG | | TTCGGCCAAGGGACCAAGGTGGAA | |
| | GGAACCCTGGTCACCGTCTCCTCAG | | ATCAAACGAACTGTGGCTGCACCA | |
| | CCTCCACCAAGGGCCCATCGGTCTT | | TCTGTCTTCATCTTCCCGCCATCTG | |
| | CCCCCTGGCACCCTCCTCCAAGAGC | | ATGAGCAGTTGAAATCTGGAACTG | |
| | ACCTCTGGGGGCACAGCGGCCCTGG | | CCTCTGTTGTGTGCCTGCTGAATAA | |
| | GCTGCCTGGTCAAGGACTACTTCCC | | CTTCTATCCCAGAGAGGCCAAAGT | |
| | CGAACCGGTGACGGTGTCGTGGAAC | | ACAGTGGAAGGTGGATAACGCCCT | |
| | TCAGGCGCCCTGACCAGCGGCGTGC | | CCAATCGGGTAACTCCCAGGAGAG | |
| | ACACCTTCCCGGCTGTCCTACAGTC | | TGTCACAGAGCAGGACAGCAAGGA | |
| | CTCAGGA | | CAGCACCTACAGCCTCAGCAGCAC | |
| | | | CCTGACGCTGAGCAAAGCAGACTA | |
| | | | CGAGA | |
| S24-178 | CAGGTGCAGCTGGTGGAGTCTGGGG | 1630 | CAGTCTGCCCTGACTCAGCCTGCCT | 1720 |
| | GAGGCGTGGTCCAGCCTGGGAGGTC | | CCGTGTCTGGGTCTCCTGGACAGTC | |
| | CCTGAGACTCTCCTGTGCAGCCTCT | | GATCACCATCTCCTGCACTGGAAC | |
| | GGATTCACCTTCAGTAGCTATGGCA | | CACCAGTGACGTTGGTGGTTATGA | |
| | TGCACTGGGTCCGCCAGGCTCCAGG | | CTATGTCTCCTGGTACCAACAGCAC | |
| | CAAGGGGCTGGAGTGGGTGGCAGTT | | CCAGGCAAAGCCCCCAAACTCATA | |
| | ATATGGTATGATGGAAGTAATAAAT | | CTTTCTGAGGTCAGTAATCGGCCCT | |
| | ATTATGCAGACTCCGTGAAGGGCCG | | CAGGGGTTTCTAATCGCTTCTCTGG | |
| | ATTCACCATCTCCAGAGACAATTCC | | CTCCAAGTCTGGCAACACGGCCTC | |
| | AAGAACACGCTGTATCTGCAAATGA | | CCTGACCATCTCTGGGCTCCAGGCT | |
| | ACAGCCTGAGAGCCGAGGACACGG | | GAGGACGAGGCTGATTATTACTGC | |
| | CTGTGTATTACTGTGCGAGAATCGA | | AGCTCATATCCAAGCAGCAGCACT | |
| | GGGATACAGCTATGGCGACGTGAG | | CTAGTCTTCGGAACTGGGACCAAG | |
| | GGTCTACTACTACTACGGTATGGAC | | GTCACCGTCTTAGGTCAGCCCAAG | |
| | GTCTGGGGCCAAGGGACCACGGTCA | | GCCAACCCCACTGTCACTCTGTTCC | |
| | CCGTCTCCTCAGCCTCCACCAAGGG | | CGCCCTCCTCTGAGGAGCTCCAAG | |
| | CCCATCGGTCTTCCCCCTGGCACCCT | | CCAACAAGGCCACACTAGTGTGTC | |
| | CCTCCAAGAGCACCTCTGGGGGCAC | | TGATCAGTGACTTCTACCCGGGAG | |
| | AGCGGCCCTGGGCTGCCTGGTCAAG | | CTGTGACAGTGGCCTGGAAGGCAG | |
| | GACTACTTCCCCGAACCGGTGACGG | | ATGGCAGCCCCGTCAAGGCGGGAG | |
| | TGTCGTGGAACTCAGGCGCCCTGAC | | TGGAGACCACCACACCCTCCAAAC | |
| | CAGCGGCGTGCACACCTTCCCGGCT | | AAAGCAACAACAAGTACGCGGCCA | |
| | GTCCTACAGTCCTCAGGA | | GCAGCTA | |
| S24-188 | CAGGTCCACCTGGTGCAGTCTGGGG | 1631 | CAGTCTGCCCTGACTCAGCCTGCCT | 1721 |
| | CTGAGGTGAAGAAGCCTGGGTCCTC | | CCGTGTCTGGGTCTCCTGGACAGTC | |
| | GGTGAAGGTCTCCTGCAAGGCTTCT | | GATCACCATCTCCTGCACTGGAAC | |
| | GGAGGCACCTTCAGCAGCTGTGCTA | | CAGCAGTGACGTTGGTGGTTATAA | |
| | TCAGCTGGGTGCGACAGGCCCCTGG | | CTATGTCTCCTGGTACCAACAGCAC | |
| | ACAAGGGCTTGAGTGGATGGGAAG | | CCAGGCAAAGCCCCCAAACTCATG | |
| | GATCATCCCTATCCTTGGTATAGCA | | ATTTATGAGGTCACTAATCGGCCCT | |
| | AACTACGCACAGAAGTTCCAGGGCA | | CAGGGGTTTCTAATCGCTTCTCTGG | |
| | GAGTCACGATTACCGCGGACAAATC | | CTCCAGGTCTGGCAACACGGCCTC | |
| | CACGAGCACAGCCTACATGGAGCTG | | CCTGACCATCTCTGGGCTCCAGGCT | |
| | AGCAGCCTGAGATCTGAGGACACG | | GAGGACGAGGCTGATTATTACTGC | |
| | GCCGTGTATTACTGTGCGAGAGGAT | | AGCTCATATACAAGCAGCAGCCTT | |
| | GGGAGTTTGGTTCGGGGAGTTATTA | | TATGTCTTCGGAACTGGGACCAAG | |
| | TCGAACTGATTACTACTACTACGCT | | GTCGCCGTCCTAGGTCAGCCCAAG | |
| | ATGGACGTCTGGGGCCAAGGGACC | | GCCAACCCCACTGTCACTCTGTTCC | |
| | ACGGTCACCGTCTCCTCAGCCTCCA | | CGCCCTCCTCTGAGGAGCTTCAAG | |
| | CCAAGGGCCCATCGGTCTTCCCCCT | | CCAACAAGGCCACACTGGTGTGTC | |
| | GGCGCCCTGCTCCAGGAGCACCTCT | | TCATAAGTGACTTCTACCCGGGAG | |
| | GGGGGCACAGCGGCCCTGGGCTGCC | | CCGTGACAGTGGCCTGGAAGGCAG | |
| | TGGTCAAGGACTACTTCCCCGAACC | | ATAGCAGCCCCGTCAAGGCGGGAG | |
| | GGTGACGGTGTCGTGGAACTCAGGC | | TGGAGACCACCAAACCCTCCAAAC | |
| | GCCCTGACCAGCGGCGTGCACACCT | | AGAGCAACAACAAGTACGCGGCCA | |
| | TCCCGGCTGTCCTACAGTCCTCAGG | | GCAGCTA | |
| | A | | | |
| S24-202 | GAAGTGCAGCTGGTGCAGTCTGGAG | 1632 | GAAATTGTGTTGACACAGTCTCCA | 1722 |
| | CAGAGGTGAAAAAGCCCGGGGAGT | | GCCACCCTGTCTTTGTCTCCAGGGG | |
| | CTCTGAGGATCTCCTGTAAGGGTTC | | AAAGAGCCACCCTCTCCTGCAGGG | |
| | TGGATACAGCTTTAGCAGCTACTGG | | CCAGTCAGAGTGTTAGCAGCTACC | |
| | ATCAGCTGGGTGCGCCAGATGCCCG | | TAGCCTGGTACCAACAGAAACCTG | |
| | GGAAAGGCCTGGAGTGGATGGGGA | | GCCAGGCTCCCAGGCTCCTCATCTA | |
| | GGATTGATCCTAGTGACTCTAACAC | | TGATGCATCCAACAGGGCCTCTGG | |
| | CAACTACAGCCCGTCCTTCCAAGGC | | CATCCCAGCCAGGTTCAGTGGCAG | |
| | CACGTCACCATCTCAGCTGACAAGT | | TGGGTCAGGGACAGACTTCACTCT | |
| | CCATCAGCACTGCCTACCTGCAGTG | | CACCATCAGCAGCCTAGAGCCTGA | |
| | GAGCAGCCTGAAGGCCTCGGACACC | | AGATTTTGCAGTTTATTACTGTCAG | |
| | GCCATGTATTACTGTGCGAGACTCT | | CAACGTCGCAACTGGCCTCTCACTT | |
| | CCGTCCGGGTATGGTTCGGGGAGTT | | TCGGCGGAGGGACCAAGGTGGAGA | |
| | ACCCCATTACGGTATGGACGTCTGG | | CCAAACGAACTGTGGCTGCACCAT | |
| | GGCCAAGGGACCACGGTCACCGTCT | | CTGTCTTCATCTTCCCGCCATCTGA | |
| | CCTCAGCCTCCACCAAGGGCCCATC | | TGAGCAGTTGAAATCTGGAACTGC | |
| | GGTCTTCCCCCTGGCACCCTCCTCCA | | CTCTGTTGTGTGCCTGCTGAATAAC | |
| | AGAGCACCTCTGGGGGCACAGCGG | | TTCTATCCCAGAGAGGCCAAAGTA | |
| | CCCTGGGCTGCCTGGTCAAGGACTA | | CAGTGGAAGGTGGATAACGC | |
| | CTTCCCCGAACCGGTGACGGTGTCG | | | |
| | TGGAACTCAGGCGCCCTGACCAGCG | | | |
| | GCGTGCACACCTTCCCGGCTGTCCT | | | |
| | ACAGTCCTCAGGA | | | |
| S24-278 | CAGGTGCAGCTGGTGCAGTCTGGGG | 1633 | GAAATTGTGTTGACGCAGTCTCCA | 1723 |
| | CTGAGGTGAAGAAGCCTGGGGCCTC | | GGCACCCTGTCTTTGTCTCCAGGGG | |
| | AGTGAAGGTCTCCTGCAAGGCTTCT | | AAAGAGCCACCCTCTCCTGCAGGG | |
| | GGATACACCTTCACCGGCTACTATA | | CCAGTCAGAGTATTAGCAGCAGCT | |
| | TGCACTGGGTGCGACAGGCCCCTGG | | ACTTAGCCTGGTACCAGCAGAAAC | |
| | ACAAGGGCTTGAGTGGATGGGATG | | CTGGCCAGGCTCCCAGGCTCCTCAT | |
| | GATCAACCCTAACAGTGGTGACACA | | CTATGGTGCATCCAGCAGGGCCAC | |
| | AACTATGCACAGAAGTTTCAGGGCT | | TGGCATCCCAGACAGGTTCAGTGG | |
| | GGGTCACCATGACCAGGGACACGTC | | CAGTGGGTCTGGGACAGACTTCAC | |
| | CCTCAGCACAGCCTACATGGAGCTG | | TCTCACCATCAGCAGACTGGAGCC | |
| | AGCAGGCTGAAATCTGACGACACG | | TGAAGATTTTGCAGTGTATTACTGT | |
| | GCCGTGTATTACTGTGCGAGAGTAG | | CAGCAGTATGGTAGCTCACTCACTT | |
| | GGGTTGGTGAATATAGTGGGAGGCA | | TCGGCGGAGGGACCAAGGTGGAGA | |
| | CTACTACTACTACGGTATGGACGTC | | TCAAACGAACTGTGGCTGCACCAT | |
| | TGGGGCCAAGGGACCACGGTCACC | | CTGTCTTCATCTTCCCGCCATCTGA | |
| | GTCTCCTCAGCCTCCACCAAGGGCC | | TGAGCAGTTGAAATCTGGAACTGC | |
| | CATCGGTCTTCCCCCTGGCACCCTCC | | CTCTGTTGTGTGCCTGCTGAATAAC | |
| | TCCAAGAGCACCTCTGGGGGCACAG | | TTCTATCCCAGAGAGGCCAAAGTA | |
| | CGGCCCTGGGCTGCCTGGTCAAGGA | | CAGTGGAAGGTGGATAACGC | |
| | CTACTTCCCCGAACCGGTGACGGTG | | | |
| | TCGTGGAACTCAGGCGCCCTGACCA | | | |
| | GCGGCGTGCACACCTTCCCGGCTGT | | | |
| | CCTACAGTCCTCAGGA | | | |
| S24-339 | GAGGTGCAGCTGGTGGAGTCTGGGG | 1634 | GAAATAGTGATGACGCAGTCTCCA | 1724 |
| | GAGGCTTGGTACAGCCAGGGCGGTC | | GCCACCCTGTCTGTGTCTCCAGGGG | |
| | CCTGAGACTCTCCTGTACAGCTTCT | | AAAGAGCCACCCTCTCCTGCAGGG | |
| | GGATTCACCTTTGGTGATTATGCTAT | | CCAGTCAGAGTGTTAGCAGCAACT | |
| | GAGCTGGTTCCGCCAGGCTCCAGGG | | TAGCCTGGTACCAGCAGAAACCTG | |
| | AAGGGGCTGGAGTGGGTAGGTTTCA | | GCCAGGCTCCCAGGCTCCTCATCTA | |
| | TTAGAAGCAAAGCTTATGGTGGGAC | | TGGTGCATCCACCAGGGCCACTGG | |
| | AACACAACACGCCGCCTCTGTGAAA | | TATCCCAGCCAGGTTCAGTGGCAG | |
| | GGCAGATTCACCATCTCAAGAGATG | | TGGGTCTGGGACAGAGTTCACTCT | |
| | ATTCCAAAAGCATCGCCTATCTGCA | | CACCATCAGCAGCCTGCAGTCTGA | |
| | AATGAACAGCCTGAAAACCGAGGA | | AGATTTTGCAGTTTATTACTGTCAG | |
| | CACAGCCGTGTATCACTGTGCTAGA | | CAGTATGATAACTGGTGGACGTTC | |
| | GATGGATATGATTGTAGTGGTGGTA | | GGCCAAGGGACCAAGGTGGAAATC | |
| | GATGCTACTCCCATATATTTGACTA | | AAACGAACTGTGGCTGCACCATCT | |
| | CTGGGGCCAGGGAACCCTGGTCACC | | GTCTTCATCTTCCCGCCATCTGATG | |
| | GTCTCCTCAGGTGAGTCCTCACCAC | | AGCAGTTGAAATCTGGAACTGCCT | |
| | CCCCTCTCTGAGTCCACTTAGGGAG | | CTGTTGTGTGCCTGCTGAATAACTT | |
| | ACTCAGCTTGCCAGGGTCTCAGGGT | | CTATCCCAGAGAGGCCAAAGTACA | |
| | CAGAGTCTTGTAG | | GTGGAAGGTGGATAACGC | |
| S24-472 | CAGGTGCAGCTGCAGGAGTCGGGCC | 1635 | CAGCTTGTGCTGACTCAATCGCCCT | 1725 |
| | CAGGACTGGTGAAGCCTTCGGGGAC | | CTGCCTCTGCCTCCCTGGGAGCCTC | |
| | CCTGTCCCTCACCTGCGCTGTCTCTG | | GGTCAAGCTCACCTGCACTCTGAG | |
| | GTGGCTCCATCAGCAGTATTAACTG | | CAGTGGGCACAGCAGCTACACCAT | |
| | GTGGAGTTGGGTCCGCCAGCCCCCA | | CGCATGGCATCAGCAGCAGCCAGA | |
| | GGGAAGGGGCTGGAGTGGATCGGG | | GAAGGGCCCTCGGTACTTGATGAA | |
| | GAAATCTATCATAGTGGGAACACCA | | AGTTAACAGTGATGGCAGCCACAC | |
| | ACTATAACCCGTCCCTCAAGAGTCG | | CAAGGGGGACGGGATCCCTGATCG | |
| | AGTCACCATATCAGGAGACAAGTCC | | CTTCTCAGGCTCCAGCTCTGGGGCT | |
| | AAGAACCAGTTCTCCCTGAAGCTGA | | GAGCGCTACCTCACCATCTCCAGC | |
| | GCTCTGTGACCGCCGCGGACACGGC | | CTCCAGTCTGAGGATGAGGCTGAC | |
| | CGTGTATTACTGTGCGAGAGGTTAC | | TATTACTGTCAGACCTGGGGCACT | |
| | TATGATAGTAGTCCTTATTACGAGC | | GGCATTCGAGTATTCGGCGGAGGG | |
| | CACAGGGAATTGACTACTGGGGCCA | | ACCAAGCTGACCGTCCTAGGTCAG | |
| | GGGAATCCTGGTCACCGTCTCCTCA | | CCCAAGGCTGCCCCCTCGGTCACTC | |
| | GCCTCCACCAAGGGCCCATCGGTCT | | TGTTCCCGCCCTCCTCTGAGGAGCT | |
| | TCCCCCTGGCACCCTCCTCCAAGAG | | TCAAGCCAACAAGGCCACACTGGT | |
| | CACCTCTGGGGGCACAGCGGCCCTG | | GTGTCTCATAAGTGACTTCTACCCG | |
| | GGCTGCCTGGTCAAGGACTACTTCC | | GGAGCCGTGACAGTGGCCTGGAAG | |
| | CCGAACCGGTGACGGTGTCGTGGAA | | GCAGATAGCAGCCCCGTCAAGGCG | |
| | CTCAGGCGCCCTGACCAGCGGCGTG | | GGAGTGGAGACCACCACACCCTCC | |
| | CACACCTTCCCGGCTGTCCTACAGT | | AAACAAAGCAACAACAAGTACGCG | |
| | CCTCAGGA | | GCCAGCAGCTA | |
| S24-490 | CAGGTGCAGCTGGTGCAGTCTGGGG | 1636 | GAAATTGTGTTGACGCAGTCTCCA | 1726 |
| | CTGAGGTGAAGAAGCCTGGGGCCTC | | GGCACCCTGTCTTTGTCTCCAGGGG | |
| | AGTGAAGGTTTCCTGCAAGGCATCT | | AAAGAGCCACCCTCTCCTGCAGGG | |
| | GGATACACCTTCACCAGCTACTTTA | | CCAGTCAGAGTGTTACCAGCAGCT | |
| | TTCACTGGGTGCGACAGGCCCCTGG | | ACTTAGCCTGGTACCAGCAGAGAC | |
| | ACAAGGGCTTGAGTGGATGGGAAT | | GTGGCCAGGCTCCCAGGCTCCTCA | |
| | AATCAACCCTAGTGGTGGTAGCACA | | TCTATGGTGCATCCAGCAGGGCCA | |
| | AGCTACGCACAGAAGTTCCAGGGCA | | CTGGCATCCCAGACAGGTTCAGTG | |
| | GAGTCACCATGACCAGGGACACGTC | | GCAGTGGGTCTGGGACAGACTTCA | |
| | CACGAGCACAGTCTACATGGAGCTG | | CTCTCACCATCAGCAGACTGGAGC | |
| | AGCAGCCTGAGATCTGAGGACACG | | CTGAAGATTTTGCAGTGTATTACTG | |
| | GCCGTGTATTACTGTGCGAGACACA | | TCAGCAGTATGGTAGCTCACCTCTC | |
| | CAACCCCGACAAGATACTTTGACTA | | ACTTTCGGCGGAGGGACCAAGGTG | |
| | CTGGGGCCAGGGAACCCTGGTCACC | | GAGATCAAACGAACTGTGGCTGCA | |
| | GTCTCCTCAGGGAGTGCATCCGCCC | | CCATCTGTCTTCATCTTCCCGCCAT | |
| | CAACCCTTTTCCCCCTCGTCTCCTGT | | CTGATGAGCAGTTGAAATCTGGAA | |
| | GAGAATTCCCCGTCGGATACGAGCA | | CTGCCTCTGTTGTGTGCCTGCTGAA | |
| | GCGTG | | TAACTTCTATCCCAGAGAGGCCAA | |
| | | | AGTACAGTGGAAGGTGGATAACGC | |
| S24-494 | CAGCTGCAGCTGCAGGAGTCGGGCC | 1637 | GACATCCAGATGACCCAGTCTCCA | 1727 |
| | CAGGACTGGTGAAGCCTTCGGAGAC | | TCCTCCCTGTCTGCATCTGTAGGAG | |
| | CCTGTCCCTCACCTGCACTGTCTCTG | | ACAGAGTCACCATCACTTGCCGGG | |
| | GTGGCTCCATCAGCAGTAGTAGTTA | | CAAGTCAGAGCATTAGCAGCTATT | |
| | CTACTGGGGCTGGATCCGCCAGCCC | | TAAATTGGTATCAGCAGAAACCAG | |
| | CCAGGGAAGGGGCTGGAGTGGATT | | GGAAAGCCCCTAAGCTCCTGATCT | |
| | GGGAGTATCTATTATAGTGGGAGCA | | ATGCTGCATCCAGTTTGCAAAGTG | |
| | CCTACTACAACCCGTCCCTCAAGAG | | GGGTCCCATCAAGGTTCAGTGGCA | |
| | TCGAGTCACCATATCCGTAGACACG | | GTGGATCTGGGACAGATTTCACTCT | |
| | TCCAAGAACCAGTTCTCCCTGAAGC | | CACCATCAGCAGTCTGCAACCTGA | |
| | TGAGCTCTGTGACCGCCGCAGACAC | | AGATTTTGCAACTTACTACTGTCAA | |
| | GGCTGTGTATTACTGTGCGAGAAAG | | CAGAGTTACAGTACCCCTCAACTC | |
| | CCACGTAGTGACTACGGGTACTTCG | | ACTTTCGGCGGAGGGACCAAGGTG | |
| | ATCTCTGGGGCCGTGGCACCCTGGT | | GAGATCAAACGAACTGTGGCTGCA | |
| | CACTGTCTCCTCAGCCTCCACCAAG | | CCATCTGTCTTCATCTTCCCGCCAT | |
| | GGCCCATCGGTC | | CTGATGAGCAGTTGAAATCTGGAA | |
| | | | CTGCCTCTGTTGTGTGCCTGCTGAA | |
| | | | TAACTTCTATCCCAGAGAGGCCAA | |
| | | | AGTACAGTGGAAGGTGGATAACGC | |
| S24-566 | GAGGTGCAGCTGGTGGAGTCTGGGG | 1638 | GATATTGTGATGACTCAGTCTCCAC | 1728 |
| | GAGGCTTGGTAAAGCCAGGGCGGTC | | TCTCCCTGCCCGTCACCCCTGGAGA | |
| | CCTGAGACTCTCCTGTACAGCTTCT | | GCCGGCCTCCATCTCCTGCAGGTCT | |
| | GGATTCACCTTTGGTGATTATGCTAT | | AGTCAGAGCCTCCTGCATAGTAAT | |
| | GAGCTGGTTCCGCCAGGCTCCAGGG | | GGATACAACTATTTGGATTGGTAC | |
| | AAGGGGCTGGAGTGGGTAGGTTTCA | | CTGCAGAAGCCAGGGCAGTCTCCA | |
| | CTAGAAGGAAAGCTTATGGTGGGAC | | CAGCTCCTGATCTATTTGGGTTCTA | |
| | AACAGAGTACGCCGCGTCTGTGAAA | | ATCGGGCCTCCGGGGTCCCTGACA | |
| | GGCAGATTCACCATCTCAAGAGATG | | GGTTCAGTGGCAGTGGATCAGGCA | |
| | ATTCCAAAAGCATCGCCTATCTGCA | | CAGATTTTACACTGAAAATCAGCA | |
| | AATGAACAGCCTGAAAACCGAGGA | | GAGTGGAGGCTGAGGATGTTGGGG | |
| | CACAGCCGTGTATTACTGTACTAGA | | TTTATTACTGCATGCAACCTCTACA | |
| | ATTAAGGTGGGCCGTTTCGATCTTA | | AACTCCTTGGACGTTCGGCCAAGG | |
| | CCGACAGTGGGAGCTACCGATACTT | | GACCAAGGTGGAAATCAAACGAAC | |
| | TGACTACTGGGGCCAGGGAACCCTG | | TGTGGCTGCACCATCTGTCTTCATC | |
| | GTCACCGTCTCCTCAGCCTCCACCA | | TTCCCGCCATCTGATGAGCAGTTGA | |
| | AGGGCCCATCGGTCTTCCCCCTGGC | | AATCTGGAACTGCCTCTGTTGTGTG | |
| | ACCCTCCTCCAAGAGCACCTCTGGG | | CCTGCTGAATAACTTCTATCCCAGA | |
| | GGCACAGCGGCCCTGGGCTGCCTGG | | GAGGCCAAAGTACAGTGGAAGGTG | |
| | TCAAGGACTACTTCCCCGAACCGGT | | GATAACGC | |
| | GACGGTGTCGTGGAACTCAGGCGCC | | | |
| | CTGACCAGCGGCGTGCACACCTTCC | | | |
| | CGGCTGTCCTACAGTCCTCAGGA | | | |
| S24-636 | GAGGTGCAGCTGGTGGAGTCTGGGG | 1639 | CAGACTGTGGTGACCCAGGAGCCA | 1729 |
| | GAGGCTTGGTCCAGCCTGGGGGGTC | | TCGTTCTCAGTGTCCCCTGGAGGGA | |
| | CCTGAGACTCTCCTGTGCAGCCTCT | | CAGTCACACTCACTTGTGGCTTGAG | |
| | GGATTCACCTTAAGTAGCTATTGGA | | CTCTGGCTCAGTCTCTACTAGTTAC | |
| | TGAGCTGGGTCCGCCAGGCTCCAGG | | TACCCCAGCTGGTACCAGCAGACC | |
| | GAAGGGGCTGGAGTGGGTGGCCAA | | CCAGGCCAGGCTCCACGCACGCTC | |
| | CATAAAGCAAGATGGAAGTGAGAA | | ATCTACAGCACAAACAAACGCTCT | |
| | ATACTATGTGGACTCTGTGAAGGGC | | TCTGGGGTCCCTGATCGCTTCTCTG | |
| | CGATTCACCATCTCCAGAGACAACG | | GCTCCATCCTTGGGAACAAAGCTG | |
| | CCAAGAACTCACTGTATCTGCAAAT | | CCCTCACCATCACGGGGGCCCAGG | |
| | GAACAGCCTGAGAGCCGAGGACAC | | CAGATGATGAATCTGATTATTACTG | |
| | GGCCGTGTATTACTGTGCGAGAGAT | | TGTGCTCTATATGGGTAGTGGCATG | |
| | CTAACTGCCACCTGGTTCGACCCCT | | TCGGTGTTCGGCGGAGGGACCAAG | |
| | GGGGCCAGGGAACCCTGGTCACCGT | | CTGACCGTCCTAGGTCAGCCCAAG | |
| | CTCCTCAGCACCCACCAAGGCTCCG | | GCTGCCCCCTCGGTCACTCTGTTCC | |
| | GATGTGTTCCCCATCATATCAGGGT | | CGCCCTCCTCTGAGGAGCTTCAAG | |
| | GCAGACACCCAAAGGATAACAGCC | | CCAACAAGGCCACACTGGTGTGTC | |
| | CTGTGGTCCTGGCATGCTTGATAAC | | TCATAAGTGACTTCTACCCGGGAG | |
| | TGGGTACCACC | | CCGTGACAGTGGCCTGGAAGGCAG | |
| | | | ATAGCAGCCCCGTCAAGGCGGGAG | |
| | | | TGGAGACCACCACACCCTCCAAAC | |
| | | | AAAGCAACAACAAGTACGCGGCCA | |
| | | | GCAGCTA | |
| S24-740 | CAGGTCCAGCTTGTGCAGTCTGGGG | 1640 | GACATCGTGATGACCCAGTCTCCA | 1730 |
| | CTGAGGTGAAGAAGCCTGGGGCCTC | | GACTCCCTGGCTGTGTCTCTGGGCG | |
| | AGTGAAGGTTTCCTGCAAGGCTTCT | | AGAGGGCCACCATCAACTGCAAGT | |
| | GGATACACCTTCACTAGCTATGCTT | | CCAGCCAGAGTGTTTTATACAGCTC | |
| | TGCATTGGGTGCGCCAGGCCCCCGG | | CAACAATAAGAACTACTTAGCCTG | |
| | ACAAAGGCTTGAGTGGATGGGATG | | GTACCAGCAGAAACCAGGACAGCC | |
| | GATCAACGCTGGCAATGGTAACACA | | TCCTAAGCTGCTCATTTACTGGGCA | |
| | AAATATTCACAGAGGTTCCAGGGCA | | TCTACCCGGGAATCCGGGGTCCCT | |
| | GAGTCACCATTATTAGGGACACATC | | GACCGATTCAGTGGCAGCGGGTCT | |
| | CGCGAGCACAACCTACATGGAGCTG | | GGGACAGATTTCACTCTCACCATC | |
| | AGCAGCCTGAGATCTGAAGACACG | | AGCAGCCTGCAGGCTGAAGATGTG | |
| | GCTGTGTATTACTGTGCGAGAGGCT | | GCAGTTTATTACTGTCAGCAATATT | |
| | ATGCCCGAGCCGGGGTTATTACTAT | | ATAGTACTCCTCCCCTCACTTTCGG | |
| | CAAAGAATCACTCCACCACTGGGGC | | CGGAGGGACCAAGGTGGAGATCAA | |
| | CAGGGCACCCTGGTCACCGTCTCCT | | ACGAACTGTGGCTGCACCATCTGT | |
| | CAGCCTCCACCAAGGGCCCATCGGT | | CTTCATCTTCCCGCCATCTGATGAG | |
| | CTTCCCCCTGGCACCCTCCTCCAAG | | CAGTTGAAATCTGGAACTGCCTCT | |
| | AGCACCTCTGGGGGCACAGCGGCCC | | GTTGTGTGCCTGCTGAATAACTTCT | |
| | TGGGCTGCCTGGTCAAGGACTACTT | | ATCCCAGAGAGGCCAAAGTACAGT | |
| | CCCCGAACCGGTGACGGTGTCGTGG | | GGAAGGTGGATAACGC | |
| | AACTCAGGCGCCCTGACCAGCGGCG | | | |
| | TGCACACCTTCCCGGCTGTCCTACA | | | |
| | GTCCTCAGGA | | | |
| S24-791 | CAGGTGCAGCTGCAGGAGTCGGGCC | 1641 | GAGATTGTGTTGACGCACTCTCCA | 1731 |
| | CAGGACTGGTGAAGCCTTCGGAGAC | | GGCACCCTGTCTTTGTCTCCAGGGG | |
| | CCTGTCCCTCACCTGCACTGTCTCTG | | AAAGAGCCACCCTCTCCTGCAGGG | |
| | GTGGCTCCATCAGTAGTTCCTACTG | | CCAGTCAGAGTGTCCGCAGCTACT | |
| | GAGCTGGATCCGGCAGCCCCCAGGG | | TAGCCTGGTACCAGCAGAAACCTG | |
| | AAGGGACTGGAGTGGATTGGGTATA | | GCCAGGCTCCCAGGCTCCTCATCTA | |
| | TCTATTACAGTGGGAACACCAACTA | | TGGTGCATCCAGCAGGGCCACTGG | |
| | CAACCCCTCCCTCAAGAGTCGAGTC | | CATCCCAGACAGGTTCAGTGGCAG | |
| | ACCCTATCAATAGACACGTCCAAGA | | TGGGTCTGGGACAGACTTCACTCTC | |
| | ACCAGTTCTCCCTGAAGCTGAGCTC | | ACCATCAGCAGACTGGAGCCTGAC | |
| | TGTGACCGCTGCGGACACGGCCGTG | | GATTTTGCAGTGTATTACTGTCAGC | |
| | TATTACTGTGCGTGCAGTGTTACGA | | AGTATGGTAGCTCACCTTGGACGTT | |
| | TTTTTGGAGTGGTTACCCCTGCTTTT | | CGGCCAAGGGACCAAGGTGGAAAT | |
| | GATATCTGGGGCCAAGGGACAATG | | CAAACGAACTGTGGCTGCACCATC | |
| | GTCACCGTCTCTTCAGCCTCCACCA | | TGTCTTCATCTTCCCGCCATCTGAT | |
| | AGGGCCCATCGGTCTTCCCCCTGGC | | GAGCAGTTGAAATCTGGAACTGCC | |
| | ACCCTCCTCCAAGAGCACCTCTGGG | | TCTGTTGTGTGCCTGCTGAATAACT | |
| | GGCACAGCGGCCCTGGGCTGCCTGG | | TCTATCCCAGAGAGGCCAAAGTAC | |
| | TCAAGGACTACTTCCCCGAACCGGT | | AGTGGAAGGTGGATAACGCCCTCC | |
| | GACGGTGTCGTGGAACTCAGGCGCC | | AATCGGGTAACTCCCAGGAGAGTG | |
| | CTGACCAGCGGCGTGCACACCTTCC | | TCACAGAGCAGGACAGCAAGGACA | |
| | CGGCTGTCCTACAGTCCTCAGGA | | GCACCTACAGCCTCAGCAGCACCC | |
| | | | TGACGCTGAGCAAAGCAGACTACG | |
| | | | AG | |
| S24-902 | CAGGTCCAGCTGGTGCAGTCTGGGG | 1642 | CAGGCTGTGGTGACTCAGGAGCCC | 1732 |
| | CTGAGGTGAAGAAGCCTGGGTCCTC | | TCACTGACTGTGTCCCCAGGAGGG | |
| | GGTGAAGGTCTCCTGCAAGGCTTCT | | ACAGTCACTCTCACCTGTGGCTCCA | |
| | GGAGGCACCTTCAGCAGCTATGCTA | | GCACTGGAGCTGTCACCAGTGGTC | |
| | TCAGCTGGGTGCGACAGGCCCCTGG | | ATTATCCCTACTGGTTCCAGCAGAA | |
| | ACAAGGGCTTGAGTGGATGGGAAG | | GCCTGGCCAAGCCCCCAGGACACT | |
| | GATCATCCCTATCCTTGGTATAGCA | | GATTTATGATACAAGCAACAAACA | |
| | AACTACGCACAGAAGTTCCAGGGCA | | CTCCTGGACACCTGCCCGGTTCTCA | |
| | GAGTCACGATTACCGCGGACAAATC | | GGCTCCCTCCTTGGGGGCAAAGCT | |
| | CACGAGCACAGCCTACATGGAGCTG | | GCCCTGACCCTTTCGGGTGCGCAG | |
| | AGCAGCCTGAGATCTGAGGACACG | | CCTGAGGATGAGGCTGAGTATTAC | |
| | GCCGTGTATTACTGTGCGAGATGGG | | TGCTTGCTCTCCTATAGTGGTTGGG | |
| | ATTTTGGAGTGGTTATTCAATACGG | | TGTTCGGCGGAGGGACCAAGCTGA | |
| | TATGGACGTCTGGGGCCAAGGGACC | | CCGTCCTAGGTCAGCCCAAGGCTG | |
| | ACGGTCACCGTCTCCTCAGCCTCCA | | CCCCCTCGGTCACTCTGTTCCCGCC | |
| | CCAAGGGCCCATCGGTCTTCCCCCT | | CTCCTCTGAGGAGCTTCAAGCCAA | |
| | GGCACCCTCCTCCAAGAGCACCTCT | | CAAGGCCACACTGGTGTGTCTCAT | |
| | GGGGGCACAGCGGCCCTGGGCTGCC | | AAGTGACTTCTACCCGGGAGCCGT | |
| | TGGTCAAGGACTACTTCCCCGAACC | | GACAGTGGCCTGGAAGGCAGATAG | |
| | GGTGACGGTGTCGTGGAACTCAGGC | | CAGCCCCGTCAAGGCGGGAGTGGA | |
| | GCCCTGACCAGCGGCGTGCACACCT | | GACCACCACACCCTCCAAACAAAG | |
| | TCCCGGCTGTCCTACAGTCCTCAGG | | CAACAACAAGTACGCGGCCAGCAG | |
| | ACTCTACTCCCTCAGCAGCGTGGTG | | CTA | |
| | ACCGTGCCCTCCAGCAGCTTGGG | | | |
| S24-921 | CAGGTGCAGCTGCAGGAGTCGGGCC | 1643 | GACATCCAGATGACCCAGTCTCCA | 1733 |
| | CAGGACTGGTGAAGCCTTCGGAGAC | | TCCTCCCTGTCTGCATCTCTGGGAG | |
| | CCTGTCCCTCACCTGCACTGTCTCTG | | ACGGGGTCACCATCACTTGCCGGG | |
| | GTGGCTCCATCAATAGTTTCTACTG | | CAAGTCAGAGCATTAGCAGCTATT | |
| | GAACTGGATCCGGCAGCCCCCCGGG | | TAAGTTGGTATCAGCAGAAACCCG | |
| | AAGGGACTGGAGTGGATTGGGTATA | | GGAAAGCCCCTAAGCTCCTGATCT | |
| | TCTATTACAGTGGGAACACCAAGTA | | ATGCTGCATCCAGTTTGCAAAGTG | |
| | CAACCCCTCCCTCAAGAGTCGAGTC | | GGGTCCCATCAAGGTTCAGTGGCA | |
| | ACCATATCAGTAGACACGTCCAACA | | GTGGATCTGGGACAGATTTCACTCT | |
| | GCCAGTTCTCCCTGAAGCTGAGCTC | | CACCATCAGCAGTCTGCAACCTGA | |
| | TGTGACCGCTGCGGACACGGCCGTG | | AGATTTTGCAACTTACTACTGTCAA | |
| | TATTACTGTGCGGCGCTCAAAAAGC | | CAGAGTTACAATACCCCCGTGACG | |
| | AGGAGCTGGTATCGTTGCAGGCTTT | | TTCGGCCAAGGGACCAAGGTGGAA | |
| | TGATATCTGGGGCCAAGGGACAATG | | ATCAAACGAACTGTGGCTGCACCA | |
| | GTCACCGTCTCTTCAGCCTCCACCA | | TCTGTCTTCATCTTCCCGCCATCTG | |
| | AGGGCCCATCGGTCTTCCCCCTGGC | | ATGAGCAGTTGAAATCTGGAACTG | |
| | ACCCTCCTCCAAGAGCACCTCTGGG | | CCTCTGTTGTGTGCCTGCTGAATAA | |
| | GGCACAGCGGCCCTGGGCTGCCTGG | | CTTCTATCCCAGAGAGGCCAAAGT | |
| | TCAAGGACTACTTCCCCGAACCGGT | | ACAGTGGAAGGTGGATAACGCAGA | |
| | GACGGTGTCGTGGAACTCAGGCGCC | | TCGGAAGAGC | |
| | CTGACCAGCGGCGTGCACACCTTCC | | | |
| | CGGCTGTCCTACAGTCCTCAGGA | | | |
| S24-1063 | CAGGTGCAGCTGCAGGAGTCGGGCC | 1644 | GAAATTGTGTTGACGCAGTCTCCA | 1734 |
| | CAGGACTGGTGAAGCCTTCGGAGAC | | GGCACCCTGTCTTTGTCTCCAGGGG | |
| | CCTGTCCCTCACCTGCACTGTCTCTG | | AAAGAGCCACCCTCTCCTGCAGGG | |
| | GTGGCTCCATCAGTAGTTACTACTG | | CCAGTCAGAGTGTTAGCAGCAGCT | |
| | GAGCTGGATCCGGCAGCCCCCAGGG | | ACTTAGCCTGGTACCAGCAGAAAC | |
| | AAGGGACTGGAGTGGATTGGATATA | | CTGGCCAGGCTCCCAGGCTCCTCAT | |
| | TCTATTACAGTGGGAGCACCAAGTA | | CTATGGTGCATCCAGCAGGGCCAC | |
| | CAACCCCTCCCTCAAGAGTCGAGTC | | TGACATCCCAGACAGGTTCAGTGG | |
| | ACCATATCAGTAGACACGTCCAAGA | | CAGTGGGTCTGGGACAGACTTCAC | |
| | ACCAGTTCTCCCTGAAGCTGACCTC | | TCTCACCATCAGCAGACTGGAGCC | |
| | TGTGACCGCTGCGGACACGGCCGTG | | TGAAGATTTTGCAGTGTATTACTGT | |
| | TATTACTGTGCGAGAATCTATGATA | | CAGCAGTATGGTAGCTCACCGTGG | |
| | GTAGTGGTTATTACCATCCCGTCTTT | | ACGTTCGGCCAAGGGACCAAGGTG | |
| | GACTACTGGGGCCAGGGAACCCTGG | | GAAATCAAACGAACTGTGGCTGCA | |
| | TCACCGTCTCCTCAGCCTCCACCAA | | CCATCTGTCTTCATCTTCCCGCCAT | |
| | GGGCCCATCGGTCTTCCCCCTGGCA | | CTGATGAGCAGTTGAAATCTGGAA | |
| | CCCTCCTCCAAGAGCACCTCTGGGG | | CTGCCTCTGTTGTGTGCCTGCTGAA | |
| | GCACAGCGGCCCTGGGCTGCCTGGT | | TAACTTCTATCCCAGAGAGGCCAA | |
| | CAAGGACTACTTCCCCGAACCGGTG | | AGTACAGTGGAAGGTGGATAACGC | |
| | ACGGTGTCGTGGAACTCAGGCGCCC | | | |
| | TGACCAGCGGCGTGCACACCTTCCC | | | |
| | GGCTGTCCTACAGTCCTCAGGA | | | |
| S24-1224 | CAGGTGCAGCTGGTGCAGTCTGGGG | 1645 | CAGTCTGTGCTGACGCAGCCGCCC | 1735 |
| | CTGAGGTGAAGAAGCCTGGGGCCTC | | TCAGTGTCTGGGGCCCCAGGGCAG | |
| | AGTGAGGGTTTCCTGCAAGGCATCT | | AGGGTCACCATCCCCTGCACTGGG | |
| | GGATACACCTTCACCAGCTACTATA | | AGCAGCTTCAACATCGGGGCAGGT | |
| | TCTACTGGGTGCGACAGGCCCCTGG | | TATGATGTACACTGGTACCAGCAG | |
| | ACAAGGGCTTGAGTGGATGGGAGT | | CTTCCAGGAACAGCCCCCAAACTC | |
| | AATCAACCCTAGTGGTGGTAGCACA | | CTCATCTTTGGTAACAGCAATCGGC | |
| | AGCTACGCACAGAAGTTCCAGGGCA | | CCTCAGGGGTCCCTGACCGATTCTC | |
| | GAGTCACCTTGACCAGGGACACGTC | | TGGCTCCAGGTCTGGCACCTCAGC | |
| | CACGAGCACAGTCTACATGGACCTG | | CTCCCTGGCCATCACTGGGCTCCAG | |
| | AGCAGTCTGAGATCTGAGGACACGG | | GCTGAGGATGAGGCTGATTATTAC | |
| | CCGTGTATTACTGTGCGAGAGATCC | | TGCCAGTCCTATGACAGTAGCCTG | |
| | TATAATGTGGGAGGTAGTAACTCGG | | AGTGGTGTGGTATTCGGCGGAGGG | |
| | GGGAGGGGCAACTGGTTCGACCCCT | | ACTACGCTGACCGTCCTAGGTCAG | |
| | GGGGCCAGGGAACCCTGGTCACCGT | | CCCAAGGCTGCCCCCTCGGTCACTC | |
| | CTCCTCAGCCTCCACCAAGGGCCCA | | TGTTCCCGCCCTCCTCTGAGGAGCT | |
| | TCGGTCTTCCCCCTGGCACCCTCCTC | | TCAAGCCAACAAGGCCACACTGGT | |
| | CAAGAGCACCTCTGGGGGCACAGC | | GTGTCTCATAAGTGACTTCTACCCG | |
| | GGCCCTGGGCTGCCTGGTCAAGGAC | | GGAGCCGTGACAGTGGCCTGGAAG | |
| | TACTTCCCCGAACCGGTGACGGTGT | | GCAGATAGCAGCCCCGTCAAGGCG | |
| | CGTGGAACTCAGGCGCCCTGACCAG | | GGAGTGGAGACCACCACACCCTCC | |
| | CGGCGTGCACACCTTCCCGGCTGTC | | AAACAAAGCAACAACAAGTACGCG | |
| | CTACAGTCCTCAGGA | | GCCAGCAGCTACCTGAGCCTGACG | |
| | | | CCTGAGCAGTGGAAGTCCCAC | |
| S24-1271 | GAGGTGCAGCTGGTGGAGTCTGGGG | 1646 | TCCTATGAGCTGACTCAGCCACCCT | 1736 |
| | GAGGCTTGGTCCAGCCTGGGGGGTC | | CAGTGTCCGTGTCCCCAGGACAGA | |
| | CCTGAGACTCTCCTGTGCAGCCTCT | | CAGCCAGCATCACCTGCTCTGGGG | |
| | GGATTCACCGTCAGTAGCAACTACA | | ATAAATTGGGGGATAGATATGTTT | |
| | TGAGCTGGGTCCGCCAGGCTCCAGG | | GTTGGTATCAGCAGAAGCCAGGTC | |
| | GAAGGGGCTGGAGTGGGTCTCAGTT | | AGTCCCCTGTGCTGGTCATCTATCA | |
| | ATTTATAGCGATGGTAACACATACT | | AGATACCAAGCGGCCCTCAGGGAT | |
| | ATGCAGACTCCGTGAAGGGCAGATT | | CCCTGAGCGATTCTCTGGCTCCAAC | |
| | CACCATCTCCAGAGACAATTCCAAG | | TCTGGGAACACAGCCACTCTGACC | |
| | AACATGTTATATCTTCAAATGAACA | | ATCAGCGGGACCCAGGCTATGGAT | |
| | GCCTGAGAGCCGAGGACACGGCTGT | | GAGGCTGACTATTACTGTCAGGCG | |
| | GTATTACTGTGCGAGAGACCCCGGC | | TGGGACAGCAGCACTTGGGTGTTC | |
| | CAGGGGTATTGTAGTGGTGGTAGCT | | GGCGGAGGGACCAAGCTGACCGTC | |
| | GCGCTCCGTCCTATTCTCTTGACTAC | | CTGGGTCAGCCCAAGGCTGCCCCC | |
| | TGGGGCCAGGGAACCCTGGTCACTG | | TCGGTCACTCTGTTCCCGCCCTCCT | |
| | TCTCCTCAGGGAGTGCATCCGCCCC | | CTGAGGAGCTTCAAGCCAACAAGG | |
| | AACCCTTTTCCCCCTCGTCTCCTGTG | | CCACACTGGTGTGTCTCATAAGTG | |
| | AGAATTCCCCGTCGGATACGAGCAG | | ACTTCTACCCGGGAGCCGTGACAG | |
| | CGTG | | TGGCCTGGAAGGCAGATAGCAGCC | |
| | | | CCGTCAAGGCGGGAGTGGAGACCA | |
| | | | CCACACCCTCCAAACAAAGCAACA | |
| | | | ACAAGTACGCGGCCAGCAGCTA | |
| S24-1339 | GAGGTGCAGCTGGTGGAGTCTGGAG | 1647 | GAAATTGTGTTGACGCAGTCTCCA | 1737 |
| | GAGGCTTGGTCCAGCCTGGGGGGTC | | GGCACCCTGTCTTTGTCTCCAGGGG | |
| | CCTGAGACTCTCCTGTGCAGCCTCT | | AAAGAGCCACCCTCTCCTGCAGGG | |
| | GGGTTCACCGTCAGTAGCAACTACA | | CCAGTCAGAGTGTTAGCAGCAGCT | |
| | TGAGCTGGGTCCGCCAGGCTCCAGG | | ACTTAGCCTGGTACCAGCAGAAAC | |
| | GAAGGGGCTGGAGTGGGTCTCAGAT | | CTGACCAGGCTCCCAGGCTCCTCAT | |
| | ATTTATAGCGGTGGTAGCACATACT | | CTATGGTGCATCCAGCAGGGCCAC | |
| | ACGCAGACTCCGTGAAGGGCCGATT | | TGGCATCCCAGACAGGTTCAGTGG | |
| | CACCATCTCCAGACACAATTCCAAG | | CAGTGGGTCTGGGACAGACTTCAC | |
| | AACACGCTGTATCTTCAAATGAACA | | TCTCACCATCAGCAGACTGGAGCC | |
| | GCCTGAGAGCTGAGGACACGGCCGT | | TGAAGATTTTGCAGTGTATTACTGT | |
| | GTATTACTGTGCGAGAGATCGACGG | | CAGCAGTATGGTAGCTCACCTAAC | |
| | GGATACAGCTATGGTTTGCACCACG | | ACTTTTGGCCAGGGGACCAAGCTG | |
| | GTATGGACGTCTGGGGCCAAGGGAC | | GAGATCAAACGAACTGTGGCTGCA | |
| | CACGGTCACCGTCTCCTCAGCCTCC | | CCATCTGTCTTCATCTTCCCGCCAT | |
| | ACCAAGGGCCCATCGGTCTTCCCCC | | CTGATGAGCAGTTGAAATCTGGAA | |
| | TGGCACCCTCCTCCAAGAGCACCTC | | CTGCCTCTGTTGTGTGCCTGCTGAA | |
| | TGGGGGCACAGCGGCCCTGGGCTGC | | TAACTTCTATCCCAGAGAGGCCAA | |
| | CTGGTCAAGGACTACTTCCCCGAAC | | AGTACAGTGGAAGGTGGATAACGC | |
| | CGGTGACGGTGTCGTGGAACTCAGG | | | |
| | CGCCCTGACCAGCGGCGTGCACACC | | | |
| | TTCCCGGCTGTCCTACAGTCCTCAG | | | |
| | GA | | | |
| S24-1345 | CAGCTGCAGCTGCAGGAGTCGGGCC | 1648 | GCCATCCAGTTGACCCAGTCTCCAT | 1738 |
| | CAGGACTGGTGAAGCCTTCGGAGAC | | CCTCCCTGTCTGCATCTGTAGGAGA | |
| | CCTGTCCCTCACCTGCACTGTCTCTG | | CAGAGTCACCATCACTTGCCGGGC | |
| | GTGGCTCCATCAGCAGTAGTAGTTA | | AAGTCAGGGCATTAGCAGTGCTTT | |
| | CTACTGGGGCTGGATCCGCCAGCCC | | AGCCTGGTATCAGCAGAAACCAGG | |
| | CCAGGGAAGGGGCTGGAGTGGATT | | GAAAGCTCCTAAGCTCCTGATCTAT | |
| | GGGAGTATCTATTATAGTGGGAGCA | | GATGCCTCCAGTTTGGAAAGTGGG | |
| | CCTACTACAACCCGTCCCTCAAGAG | | GTCCCATCAAGGTTCAGCGGCAGT | |
| | TCGAGTCACCATATCCGTAGACACG | | GGATCTGGGACAGATTTCACTCTC | |
| | TCCAAGAACCAGTTCTCCCTGAAGC | | ACCATCAGCAGCCTGCAGCCTGAA | |
| | TGAGCTCTGTGACCGCCGCAGACAC | | GATTTTGCAACTTATTACTGTCAAC | |
| | GGCTGTGTATTACTGTGCGAGACGA | | AGTTTAATAGTTACCTCACTTTCGG | |
| | ATCAGACGCCCCACCTCGGAAGTGG | | CGGAGGGACCAAGGTGGAGATCAA | |
| | TTATTACTTATGTCTTTGACTACTGG | | ACGAACTGTGGCTGCACCATCTGT | |
| | GGCCAGGGAACCCTGGTCACCGTCT | | CTTCATCTTCCCGCCATCTGATGAG | |
| | CCTCAGCACCCACCAAGGCTCCGGA | | CAGTTGAAATCTGGAACTGCCTCT | |
| | TGTGTTCCCCATCATATCAGGGTGC | | GTTGTGTGCCTGCTGAATAACTTCT | |
| | AGACACCCAAAGGATAACAGCCCT | | ATCCCAGAGAGGCCAAAGTACAGT | |
| | GTGGTCCTGGCATGCTTGATAACTG | | GGAAGGTGGATAACGCCCTCCAAT | |
| | GGTACCACC | | CGGGTAACTCCCAGGAGAGTGTCA | |
| | | | CAGAGCAGGACAGCAAGGACAGC | |
| | | | ACCTACAGCCTCAGC | |
| S24- | GAGGTGCAGCTGGTGGAGTCTGGAG | 1649 | CAGACTGTGGTGACCCAGGAGCCA | 1739 |
| 1378 | GAGGCTTGGTCCAGCCTGGGGGGTC | | TCGTTCTCAGTGTCCCCTGGAGGGA | |
| | CCTGAGACTCTCCTGTGCAGCCTCT | | CAGTCACACTCACTTGTGGCTTGAG | |
| | GGGTTCACCGTCAGTAGCAACTACA | | CTCTGGCTCAGTCTCTACTAGTTAC | |
| | TGAGCTGGGTCCGCCAGGCTCCAGG | | TACCCCAGCTGGTACCAGCAGACC | |
| | GAAGGGGCTGGAGTGGGTCTCAGTT | | CCAGGCCAGGCTCCACGCACGCTC | |
| | ATTTATAGCGGTGGTAGCACATACT | | ATCTACAGCACAAACACTCGCTCTT | |
| | ACGCAGACTCCGTGAAGGGCCGATT | | CTGGGGTCCCTGATCGCTTCTCTGG | |
| | CACCATCTCCAGACACAATTCCAAG | | CTCCATCCTTGGGAACAAAGCTGC | |
| | AACACGCTGTATCTTCAAATGAACA | | CCTCACCATCACGGGGGCCCAGGC | |
| | GCCTGAGAGCTGAGGACACGGCCGT | | AGATGATGAATCTGATTATTACTGT | |
| | GTATTACTGTGCGAGAGAAGGATAT | | GTGCTGTATATGGGTAGTGGCATTT | |
| | TGTACTAATGGTGTATGCTATAGGC | | CGGTGTTCGGCGGAGGGACCAAGC | |
| | ATGCTTTTGATATCTGGGGCCAAGG | | TGACCGTCCTAGGTCAGCCCAAGG | |
| | GACAATGGTCACCGTCTCTTCAGGG | | CTGCCCCCTCGGTCACTCTGTTCCC | |
| | AGTGCATCCGCCCCAACCCTTTTCC | | GCCCTCCTCTGAGGAGCTTCAAGC | |
| | CCCTCGTCTCCTGTGAGAATTCCCC | | CAACAAGGCCACACTGGTGTGTCT | |
| | GTCGGATACGAGCAGCGTG | | CATAAGTGACTTCTACCCGGGAGC | |
| | | | CGTGACAGTGGCCTGGAAGGCAGA | |
| | | | TAGCAGCCCCGTCAAGGCGGGAGT | |
| | | | GGAGACCACCACACCCTCCAAACA | |
| | | | AAGCAACAACAAGTACGCGGCCAG | |
| | | | CAGCTA | |
| S24-1379 | CAGGTGCAGCTGCAGGAGTCGGGCC | 1650 | CAGTCTGTGCTGACTCAGCCACCCT | 1740 |
| | CAGGACTGGTGAAGCCTTCGGAGAC | | CAGCGTCTGGGACCCCCGGGCAGA | |
| | CCTGTCCCTCACCTGCACTGTCTCTG | | GGGTCACCATCTCTTGTTCTGGAAG | |
| | GTGGCTCCATCAGTAGTTACTACTG | | CAGCTCCAACATCGGAAGTAATTA | |
| | GAGCTGGATCCGGCAGCCCCCAGGG | | TGTATACTGGTACCAGCAGCTCCC | |
| | AAGGGACTGGAGTGGATTGGGTATA | | AGGAACGGCCCCCAAACTCCTCAT | |
| | TCTATTACAGTGGGAGCACCAACTA | | CTATAGGAATAATCAGCGGCCCTC | |
| | CAACCCCTCCCTCAAGAGTCGAGTC | | AGGGGTCCCTGACCGATTCTCTGG | |
| | ACCATATCAGTAGACACGTCCAAGA | | CTCCAAGTCTGGCACCTCAGCCTCC | |
| | ACCAGTTCTCCCTGAAGCTGAGCTC | | CTGGCCATCAGTGGGCTCCGGTCC | |
| | TGTGACCGCTGCGGACACGGCCGTG | | GAGGATGAGGCTGATTATTACTGT | |
| | TATTACTGTGCGAGAGATTACTATC | | GCAGCATGGGATGACAGCCTGAGT | |
| | AACTCCCTATGGACGTCTGGGGCCA | | GGTCGGGTGTTCGGCGGAGGGACC | |
| | AGGGACCACGGTCACCGTCTCCTCA | | AAGCTGACCGTCCTAGGTCAGCCC | |
| | GCCTCCACCAAGGGCCCATCGGTCT | | AAGGCTGCCCCCTCGGTCACTCTGT | |
| | TCCCCCTGGCACCCTCCTCCAAGAG | | TCCCGCCCTCCTCTGAGGAGCTTCA | |
| | CACCTCTGGGGGCACAGCGGCCCTG | | AGCCAACAAGGCCACACTGGTGTG | |
| | GGCTGCCTGGTCAAGGACTACTTCC | | TCTCATAAGTGACTTCTACCCGGGA | |
| | CCGAACCGGTGACGGTGTCGTGGAA | | GCCGTGACAGTGGCCTGGAAGGCA | |
| | CTCAGGCGCCCTGACCAGCGGCGTG | | GATAGCAGCCCCGTCAAGGCGGGA | |
| | CACACCTTCCCGGCTGTCCTACAGT | | GTGGAGACCACCACACCCTCCAAA | |
| | CCTCAGGA | | CAAAGCAACAACAAGTACGCGGCC | |
| | | | AGCAGCTA | |
| S24-1384 | GAGGTGCAGCTGGTGGAGTCTGGGG | 1651 | TCCTATGTGCTGACTCAGCCACCCT | 1741 |
| | GAGGCTTGGTACAGCCTGGGGGGTC | | CGGTGTCAGTGGCCCCAGGACAGA | |
| | CCTGAGACTCTCCTGTGCAGTCTCT | | CGGCCAGGATTACCTGTGGGGGAG | |
| | GGATTCACCTTCAGTAGCTATAGCA | | ACAACATTGGAAGTAAAAATGTGC | |
| | TGAACTGGGTCCGCCAGGCTCCAGG | | ACTGGTACCAGCAGAAGCCCGGCC | |
| | GAAGGGGCTGGAGTGGGTTTCATAC | | AGGCCCCTGTGCTGGTCGTCTTTGA | |
| | ATTAGTAGTAGTAGTAGTATCATAT | | TGATAGCGACCGGCCCTCAGGGAT | |
| | ACTACGCAGACTCTGTGAAGGGCCG | | CCCTGAGCGATTCTCTGGCTCCAAC | |
| | ATTCACCATCTCCAGAGACAACGCC | | TCTGGGAACACGGCCACCCTGACC | |
| | AAGAACTCACTGTATCTGCAAATGA | | ATCAGCAGGGTCGAAGCCGGGGAT | |
| | ACAGCCTGAGAGCCGAGGACACGG | | GAGGCCGACTATTACTGTCAGGTG | |
| | CTGTGTATTACTGTGCGAGAGATTT | | TGGGATAGTAGTAGTGATCACTAT | |
| | CCTCGACTATAGCAGGTCGTATTCG | | GTGGTATTCGGCGGAGGGACCAAG | |
| | TACGGTATGGACGTCTGGGGCCAAG | | CTGACCGTCCTAGGTCAGCCCAAG | |
| | GGACCACGGTCACCGTCTCCTCAGC | | GCTGCCCCCTCGGTCACTCTGTTCC | |
| | CTCCACCAAGGGCCCATCGGTCTTC | | CGCCCTCCTCTGAGGAGCTTCAAG | |
| | CCCCTGGCACCCTCCTCCAAGAGCA | | CCAACAAGGCCACACTGGTGTGTC | |
| | CCTCTGGGGGCACAGCGGCCCTGGG | | TCATAAGTGACTTCTACCCGGGAG | |
| | CTGCCTGGTCAAGGACTACTTCCCC | | CCGTGACAGTGGCCTGGAAGGCAG | |
| | GAACCGGTGACGGTGTCGTGGAACT | | ATAGCAGCCCCGTCAAGGCGGGAG | |
| | CAGGCGCCCTGACCAGCGGCGTGCA | | TGGAGACCACCACACCCTCCAAAC | |
| | CACCTTCCCGGCTGTCCTACAGTCCT | | AAAGCAACAACAAGTACGCGGCCA | |
| | CAGGA | | GCAGCTACC | |
| S24-1476 | GAGGTGCAGCTGGTGGAGTCTGGGG | 1652 | GAAATAGTGATGACGCAGTCTCCA | 1742 |
| | GAGGCTTGGTACAGCCAGGGCGGTC | | GCCACCCTGTCTGTGTCTCCAGGGG | |
| | CCTGAGACTCTCCTGTACAGCTTCT | | AAAGAGCCACCCTCTCCTGCAGGG | |
| | GGATTCACCTTTGGTGATTATGCTAT | | CCAGTCAGAGTGTTAGCAGCAACT | |
| | GAGCTGGTTCCGCCAGGCTCCAGGG | | TAGCCTGGTACCAGCAGAAACCTG | |
| | AAGGGGCTGGAGTGGGTAGGTTTCA | | GCCAGGCTCCCAGGCTCCTCATCTA | |
| | TTAGAAGCAAAGCTTATGGTGGGAC | | TGGTGCATCCACCAGGGCCACTGG | |
| | AACACAATACGCCGCCTCTGTGAAA | | TATCCCAGCCAGGTTCAGTGGCAG | |
| | GGCAGATTCACCATCTCAAGAGATG | | TGGGTCTGGGACAGAGTTCACTCT | |
| | ATTCCAAAAGCATCGCCTATCTGCA | | CACCATCAGCAGCCTGCAGTCTGA | |
| | AATGAACAGCCTGAAAACCGAGGA | | AGATTTTGCAGTTTATTACTGTCAG | |
| | CACAGCCGTGTATTACTGTACTAGA | | CAGTATAATAACTGGTGGACGTTC | |
| | GTACGATATTGTACTAATGGTGTAT | | GGCCAAGGGACCAAGGTGGAAATC | |
| | GCTATGGCTACCACTTTGACTACTG | | AAACGAACTGTGGCTGCACCATCT | |
| | GGGCCAGGGAACCGTGGTCACCGTC | | GTCTTCATCTTCCCGCCATCTGATG | |
| | TCCTCAGCCTCCACC | | AGCAGTTGAAATCTGGAACTGCCT | |
| | | | CTGTTGTGTGCCTGCTGAATAACTT | |
| | | | CTATCCCAGAGAGGCCAAAGTACA | |
| | | | GTGGAAGGTGGATAACGC | |
| S24-1564 | CAGGTGCAGCTGCAGGAGTCGGGCC | 1653 | GACATCCAGATGACCCAGTCTCCA | 1743 |
| | CAGGACTGGTGAAGCCTTCGGAGAC | | TCCTCCCTGTCTGCATCTGTAGGAG | |
| | CCTGTCCCTCACCTGCACTGTCTCTG | | ACCGGGTCACCATCACTTGCCGGG | |
| | GTGGCTCCATCAGTAGTTACTACTG | | CAAGTCAGAGCATTAGAAGCTATT | |
| | GAGCTGGATCCGTCAGCCCCCAGGG | | TAAATTGGTATCAGCAGAAACGAG | |
| | AAGGGGCTGGAGTGGATTGGCTATG | | GGAAAGCCCCTAAGCTCCTGATCT | |
| | TCTATTACAGTGGGAACACCAAATA | | ATGCTGCATCCAGTTTGCAAAGTG | |
| | CAACCCCTCCCTCAAGAGTCGAGTC | | GGGTCCCATCAAGGTTCAGTGGCA | |
| | ACCATATCAGTAGACACGTCCAAGA | | GTGGATCTGGGACAGATTTCACTCT | |
| | ACCAGTTCTCCCTGAAGCTGGGCTC | | CACCATCAGCAGTCTGCAACCTGA | |
| | TGTGACCGCCGCGGACACGGCCGTT | | AGATTTTGCAACTTACTACTGTCAA | |
| | TATTATTGTGCGAGACATTCGAGGA | | CAGAGTTACAGTACCCCTCCGACG | |
| | TAGAAGTGGCTGGTACTCTAGACTT | | TTCGGCCAAGGGACCAAGGTGGAA | |
| | TGACTACTGGGGCCAGGGAACCCTG | | ATCAAACGAACTGTGGCTGCACCA | |
| | GTCACCGTCTCCTCAGCCTCCACCA | | TCTGTCTTCATCTTCCCGCCATCTG | |
| | AGGGCCCATCGGTCTTCCCCCTGGC | | ATGAGCAGTTGAAATCTGGAACTG | |
| | ACCCTCCTCCAAGAGCACCTCTGGG | | CCTCTGTTGTGTGCCTGCTGAATAA | |
| | GGCACAGCGGCCCTGGGCTGCCTGG | | CTTCTATCCCAGAGAGGCCAAAGT | |
| | TCAAGGACTACTTCCCCGAACCGGT | | ACAGTGGAAGGTGGATAACGC | |
| | GACGGTGTCGTGGAACTCAGGCGCC | | | |
| | CTGACCAGCGGCGTGCACACCTTCC | | | |
| | CGGCTGTCCTACAGTCCTCAGGA | | | |
| S24-1636 | CAGGTGCAGCTGGTGGAGTCTGGGG | 1654 | GAAATTGTGTTGACACAGTCTCCA | 1744 |
| | GAGGCGTGGTCCAGCCTGGGAGGTC | | GCCACCCTGTCTTTGTCTCCAGGGG | |
| | CCTGAGACTCTCCTGTGCAGCCTCT | | AAAGAGCCACCCTCTCCTGCAGGG | |
| | GGATTCACCTTCAGTAACTATGGCA | | CCAGTCAGAGTGTTAGCAGCTACT | |
| | TGCACTGGGTCCGCCAGGCTCCAGG | | TAGCCTGGTACCAACAGAAACCTG | |
| | CAAGGGGCTGGAGTGGGTGGCCGTT | | GCCAGGCTCCCAGGCTCCTCATCTA | |
| | ATATGGTATGATGGAAGTAATAAAT | | TGATGCATCCAACAGGGCCACTGG | |
| | ACTATGCAGACTCCGTGAAGGGCCG | | CATCCCAGCCAGGTTCAGTGGCAG | |
| | ATTCACCATCTCCAGAGACAATTCC | | TGGGTCTGGGACAGACTTCACTCTC | |
| | AAGAACACGCTGTATCTGCAAATGA | | ACCATCAGCAGCCTAGAGCCTGAA | |
| | ACAGCCTGAGAGCCGAGGACACGG | | GATTTTGCAGTTTATTACTGTCAGC | |
| | CTGTGTATTACTGTGCGAGAGGAGA | | AGCGTAGCAACTGGCCTCCGATCA | |
| | TTGTACTAATGGTGTATGCCATCCC | | CTTTCGGCCCTGGGACCAAAGTGG | |
| | CTTCTAATTTATTATGATAGTAGTGG | | ATATCAAACGAACTGTGGCTGCAC | |
| | TTTAGACTACTGGGGCCAGGGAACC | | CATCTGTCTTCATCTTCCCGCCATC | |
| | CTGGTCACCGTCTCCTCAGCCTCCA | | TGATGAGCAGTTGAAATCTGGAAC | |
| | CCAAGGGCCCATCGGTCTTCCCCCT | | TGCCTCTGTTGTGTGCCTGCTGAAT | |
| | GGCACCCTCCTCCAAGAGCACCTCT | | AACTTCTATCCCAGAGAGGCCAAA | |
| | GGGGGCACAGCGGCCCTGGGCTGCC | | GTACAGTGGAAGGTGGATAACGCC | |
| | TGGTCAAGGACTACTTCCCCGAACC | | CTCCAATCGGGTAACTCCCAGGAG | |
| | GGTGACGGTGTCGTGGAACTCAGGC | | AGTGTCACAGAGCAGGACAGCAAG | |
| | GCCCTGACCAGCGGCGTGCACACCT | | GACAGCACCTACAGCCTC | |
| | TCCCGGCTGTCCTACAGTCCTCAGG | | | |
| | A | | | |
| S24-1002 | CAGGTGCAGCTGGTGGAGTCTGGGG | 1655 | GCCATCCAGTTGACCCAGTCTCCAT | 1745 |
| | GAGGCGTGGTCCAGCCTGGGAGGTC | | CCTCCCTGTCTGCATCTGTAGGAGA | |
| | CCTGAGACTCTCCTGTGCAGCCTCT | | CAGAGTCACCATCACTTGCCGGGC | |
| | GGATTCACCTTCACTAGCTATGCTA | | AAGTCAGGGCATTAGCAGTGCTTT | |
| | TGCACTGGGTCCGCCAGGCTCCAGG | | AGCCTGGTATCAGCAGACACCAGG | |
| | CAAGGGGCTGGAGTGGGTGGCAGTT | | GAAAGCTCCTAAGCTCCTGATCTAT | |
| | ATATCATATGATGGAGGCAGTAAAT | | GATGCCTCCAGTTTGGAAAGTGGG | |
| | ACTACGCAGACTCCGTGAAGGGCCG | | GTCCCGTCAAGGTTCAGCGGCAGT | |
| | ATTCACCATCTCCAGAGACAATTCC | | GGATCTGGGACAGATTTCTCTCTCA | |
| | AAGAACACGCTGTATCTGCAAATGA | | CCATCGGCAGCCTGCAGCCTGAAG | |
| | ACAGCCTGAGAGCTGAGGACACGG | | ATTTTGCAAGTTATTACTGTCAACA | |
| | CTGTGTATTACTGTGCGAGGACTAC | | GTTTAATAGTTACCCTCTCACTTTC | |
| | ACCGGGTATAACAGCAGCTGGAAC | | GGCGGAGGGACCAAGGTGGAGATC | |
| | AGGGACCCTAGGGAGATACTACTAC | | AAACGAACTGTGGCTGCACCATCT | |
| | TACGGTATGGACGTCTGGGGCCAAG | | GTCTTCATCTTCCCGCCATCTGATG | |
| | GGACCACGGTCACCGTCTCCTCAGG | | AGCAGTTGAAATCTGGAACTGCCT | |
| | GAGTGCATCCGCCCCAACCCTTTTC | | CTGTTGTGTGCCTGCTGAATAACTT | |
| | CCCCTCGTCTCCTGTGAGAATTCCCC | | CTATCCCAGAGAGGCCAAAGTACA | |
| | GTCGGATACGAGCAGCGTG | | GTGGAAGGTGGATAACGCCCTCCA | |
| | | | ATCGGGTAACTCCCAGGAGAGTGT | |
| | | | CACAGAGCAGGACAGCAAGGACA | |
| | | | GCACCTACAGCCTCAGCAGCACCC | |
| | | | TGACGCTGAGCAAAGCAGACTACG | |
| | | | AGA | |
| S24-1301 | CAGGTCCAACTGGTACAGTCTGGGG | 1656 | CAGGCAGGGCTGACTCAGCCACCC | 1746 |
| | CTGAGGTGAAGAAGCCTGGGGCCTC | | TCGGTGTCCAAGGGCTTGAGACAG | |
| | AGTGAAGGTCTCCTGCAAGGTTTCC | | ACCGCCACACTCACCTGCACTGGG | |
| | GGATACACCCTCATTGAATTATCCA | | AGCAGCAACAATGTTGGCAACCAA | |
| | TGCACTGGGTGCGACAGGCTCCTGG | | GGAGCAGCTTGGTTGCAGCAGCAC | |
| | AAAAGGGCTTGAGTGGATGGGAGG | | CAGGGCCACCCTCCCAAACTCCTA | |
| | TTTTGATCCTGAAGATGGTGAAACA | | TCCTACAGGAATAACAACCGGCCC | |
| | ATCTACGCACAGAAGTTCCAGGGCA | | TCAGGGATCTCAGAGAGATTCTCT | |
| | GAGTCACCATGACCGAGGACACATC | | GCATCCAGGTCAGGAAACACAGCC | |
| | TACAGACACAGCCTACATGGCGCTG | | TCCCTGACCATTACTGGACTCCAGC | |
| | AGCAGCCTGACATCTGAGGACACGG | | CTGAGGACGAGGCAGACTATTACT | |
| | CCGTGTATTACTGTGCAACAGCCTA | | GCTCAGCATGGGACAGCAGCCTCT | |
| | CGCGTATTACTATGCTTCGGGGGGT | | CTAATTGGGTGTTCGGCGGAGGGA | |
| | TATTATACCCTTGACTACTGGGGCC | | CCAAGCTGACCGTCCTAGGTCAGC | |
| | AGGGAACCCTGGTCACCGTCTCCTC | | CCAAGGCTGCCCCCTCGGTCACTCT | |
| | AGCCTCCACCAAGGGCCCATCGGTC | | GTTCCCGCCCTCCTCTGAGGAGCTT | |
| | TTCCCCCTGGCACCCTCCTCCAAGA | | CAAGCCAACAAGGCCACACTGGTG | |
| | GCACCTCTGGGGGCACAGCGGCCCT | | TGTCTCATAAGTGACTTCTACCCGG | |
| | GGGCTGCCTGGTCAAGGACTACTTC | | GAGCCGTGACAGTGGCCTGGAAGG | |
| | CCCGAACCGGTGACGGTGTCGTGGA | | CAGATAGCAGCCCCGTCAAGGCGG | |
| | ACTCAGGCGCCCTGACCAGCGGCGT | | GAGTGGAGACCACCACACCCTCCA | |
| | GCACACCTTCCCGGCTGTCCTACAG | | AACAAAGCAACAACAAGTACGCGG | |
| | TCCTCAGGA | | CCAGCAGCTA | |
| S24-223 | CAGATCACCTTGAAGGAGTCTGGTC | 1657 | CAGTCTGCCCTGACTCAGCCTGCCT | 1747 |
| | CTACGCTGGTGAAACCCACACAGAC | | CCGTGTCTGGGTCTCCTGGACAGTC | |
| | CCTCACGCTGACCTGCACCTTCTCTG | | GATCACCATCTCCTGCACTGGAAC | |
| | GGTTCTCACTCAACACTAGTGGAGT | | CAGCAGTGACGTTGGTGGTTATAA | |
| | GGGTGTGGGCTGGATCCGTCAGCCC | | CTATGTCTCCTGGTACCAACAACAC | |
| | CCAGGAAAGGCCCTGGAGTGGCTTG | | CCAGGCAAAGCCCCCAAACTCATG | |
| | CACTCATTTATTGGGATGATGATAA | | ATTTATGATGTCAGTAATCGGCCCT | |
| | GCGCTACAGCCCATCTCTGAAGAGC | | CAGGGGTTTCTAATCGCTTCTCTGG | |
| | AGGCTCACCATCACCAAGGACACCT | | CTCCAAGTCTGGCAACACGGCCTC | |
| | CCAAAAACCAGGTGGTCCTTACAAT | | CCTGACCATCTCTGGGCTCCAGGCT | |
| | GACCAACATGGACCCTGTGGACACA | | GAGGACGAGGCTGATTATTACTGC | |
| | GCCACATATTACTGTGCACACCATA | | AACTCATATACAAGCAGCAGCACT | |
| | CGATTGTTCCAATTTTTGACTACTGG | | CTCGTGGTATTCGGCGGAGGGACC | |
| | GGCCAGGGAACCCTGGTCACCGTCT | | AAGCTGACCGTCCTAGGTCAGCCC | |
| | CCTCAGGGAGTGCATCCGCCCCAAC | | AAGGCTGCCCCCTCGGTCACTCTGT | |
| | CCTTTTCCCCCTCGTCTCCTGTGAGA | | TCCCGCCCTCCTCTGAGGAGCTTCA | |
| | ATTCCCCGTCGGATACGAGCAGCGT | | AGCCAACAAGGCCACACTGGTGTG | |
| | G | | TCTCATAAGTGACTTCTACCCGGGA | |
| | | | GCCGTGACAGTGGCCTGGAAGGCA | |
| | | | GATAGCAGCCCCGTCAAGGCGGGA | |
| | | | GTGGAGACCACCACACCCTCCAAA | |
| | | | CAAAGCAACAACAAGTACGCGGCC | |
| | | | AGCAGCTATCTGAGCCTGACGCC | |
| S24-461 | CAGGTGCAGCTGCAGGAGTCGGGCC | 1658 | TCCTATGAGCTGACACAGCCACCC | 1748 |
| | CAGGACTGGTGAAGCCTTCGGAGAC | | TCGGTGTCAGTGTCCCTAGGACAG | |
| | CCTGTCCCTCACGTGCACTGTCTCTG | | ATGGCCAGGATCACCTGCTCTGGA | |
| | GTGGCTCCATCAGTAGTTACTACTG | | GAAGCATTGCCAAAAAAATATGCT | |
| | GAGCTGGATCCGGCAGCCCCCCGGG | | TATTGGTACCAGCAGAAGCCAGGC | |
| | AAGGGACTGGAGTGGATTGGGAAT | | CAGTTCCCTATACTGGTGATATATA | |
| | ATCTATAACAGTGGGAGCACCAACT | | AAGACAGCGAGAGGCCCTCAGGGA | |
| | ACAACCCCTCCCTCAAGAGTCGACT | | TCCCTGAGCGATTCTCTGGCTCCAG | |
| | CACCATATCAGTTGACACGTCCAAG | | CTCAGGGACAATAGTCACATTGAC | |
| | AACCACTTCTCCCTGAAGCTGAGCT | | CATCAGTGGAGTCCAGGCAGAAGA | |
| | CTGTGACCGCTGCGGACACGGCCGT | | CGAGGCTGACTATTACTGTCTATCA | |
| | GTATTACTGTGCGAGAGGAGGACTA | | GAAGACAGCAGTGGTACTTGGGTG | |
| | GAGCACGACGGTGACTACGTCTACT | | TTCGGCGGAGGGACCAAGCTGACC | |
| | ACTACGGTATGGACGTCTGGGGCCA | | GTCCTAGGTCAGCCCAAGGCTGCC | |
| | AGGGACCACGATCACCGTCTCCTCA | | CCCTCGGTCACTCTGTTCCCGCCCT | |
| | GCCTCCACCAAGGGCCCATCGGTCT | | CCTCTGAGGAGCTTCAAGCCAACA | |
| | TCCCCCTGGCACCCTCCTCCAAGAG | | AGGCCACACTGGTGTGTCTCATAA | |
| | CACCTCTGGGGGCACAGCGGCCCTG | | GTGACTTCTACCCGGGAGCCGTGA | |
| | GGCTGCCTGGTCAAGGACTACTTCC | | CAGTGGCCTGGAAGGCAGATAGCA | |
| | CCGAACCGGTGACGGTGTCGTGGAA | | GCCCCGTCAAGGCGGGAGTGGAGA | |
| | CTCAGGCGCCCTGACCAGCGGCGTG | | CCACCACACCCTCCAAACAAAGCA | |
| | CACACCTTCCCGGCTGTCCTACAGT | | ACAACAAGTACGCGGCCAGCAGCT | |
| | CCTCAGGA | | A | |
| S24-511 | CAGGTGCAGCTGGTGGAGTCTGGGG | 1659 | TCCTATGAGCTGACTCAGCCACCCT | 1749 |
| | GAGGCGTGGTCCAGCCTGGGAGGTC | | CAGTGTCCGTGTCCCCAGGACAGA | |
| | CCTGAGACTCTCCTGTGCAGCCTCT | | CAGCCAGCATCACCTGCTCTGGAG | |
| | GGATTCACCTTCAGTAGCTATGGCA | | ATAAATTGGGGGATAAATATGCTT | |
| | TGCACTGGGTCCGCCAGGCTCCAGG | | GCTGGTATCAGCAGAAGCCAGGCC | |
| | CAAGGGGCTGGAGTGGGTGGCAGTT | | AGTCCCCTGTGCTGGTCATCTATCA | |
| | ATATCATATGATGGAAGTAATAAAT | | AGATAGCAAGCGGCCCTCAGGGAT | |
| | ACTATGCAGACTCCGTGAAGGGCCG | | CCCTGAGCGATTCTCTGGCTCCAAC | |
| | ATTCACCATCTCCAGAGACAATTCC | | TCTGGGAACACAGCCACTCTGACC | |
| | AAGAACACGCTGTATCTGCAAATGA | | ATCAGCGGGACCCAGGCTATGGAT | |
| | ACAGCCTGAGAGCTGAGGACACGG | | GAGGCTGACTATTACTGTCAGGCG | |
| | CTGTGTATTACTGTGCGAAATATAC | | TGGGACAGCAGCACTGTGGTATTC | |
| | GTCAACGGTAACTACGAACTACTAC | | GGCGGAGGGACCAAGCTGACCGTC | |
| | TACGGTATGGACGTCTGGGGCCAAG | | CTAGGTCAGCCCAAGGCTGCCCCC | |
| | GGACCACGGTCACCGTCTCCTCAGC | | TCGGTCACTCTGTTCCCGCCCTCCT | |
| | ACCCACCAAGGCTCCGGATGTGTTC | | CTGAGGAGCTTCAAGCCAACAAGG | |
| | CCCATCATATCAGGGTGCAGACACC | | CCACACTGGTGTGTCTCATAAGTG | |
| | CAAAGGATAACAGCCCTGTGGTCCT | | ACTTCTACCCGGGAGCCGTGACAG | |
| | GGCATGCTTGATAACTGGGTACCAC | | TGGCCTGGAAGGCAGATAGCAGCC | |
| | C | | CCGTCAAGGCGGGAGTGGAGACCA | |
| | | | CCACACCCTCCAAACAAAGCAACA | |
| | | | ACAAGTACGCGGCCAGCAGCTACC | |
| S24-788 | CAGGTGCAGCTGGTGGAGTCTGGGG | 1660 | TCCTATGAGCTGACTCAGCCACCCT | 1750 |
| | GAGGCGTGGTCCAGCCTGGGAGGTC | | CAGTGTCCGTGTCCCCAGGACAGA | |
| | CCTGAGACTCTCCTGTGCAGCGTCT | | CAGCCAGCATCACCTGCTCTGGAG | |
| | GGATTCACCTTCAGTAGCTATGGCA | | ATAAATTGGGGGATAAATATGCTT | |
| | TGCACTGGGTCCGCCAGGCTCCAGG | | GCTGGTATCAGCAGAAGCCAGGCC | |
| | CAAGGGGCTGGAGTGGGTGGCAGTT | | AGTCCCCTGTGCTGGTCATCTATCA | |
| | ATATGGTATGATGGAAGTAATAAAT | | AGATAGCAAGCGGCCCTCAGGGAT | |
| | ACTATGCAGACTCCGTGAAGGGCCG | | CCCTGAGCGATTCTCTGGCTCCAAC | |
| | ATTCACCATCTCCAGAGACAATTCC | | TCTGGGAACACAGCCACTCTGACC | |
| | AAGAACACGCTGTATCTGCAAATGA | | ATCAGCGGGACCCAGGCTATGGAT | |
| | ACAGCCTGAGAGCCGAGGACACGG | | GAGGCTGACTATTACTGTCAGGCG | |
| | CTGTGTATTACTGTGCGAGAGGACG | | TGGGACAGCAGCTCTGTGGTATTC | |
| | TTCCCCAGGTGGGGGCCACTACTAC | | GGCGGAGGGACCAAGCTGACCGTC | |
| | GGTATGGACGTCTGGGGCCAAGGG | | CTAGGTCAGCCCAAGGCTGCCCCC | |
| | ACCACGGTCACCGTCTCCTCAGGGA | | TCGGTCACTCTGTTCCCGCCCTCCT | |
| | GTGCATCCGCCCCAACCCTTTTCCCC | | CTGAGGAGCTTCAAGCCAACAAGG | |
| | CTCGTCTCCTGTGAGAATTCCCCGTC | | CCACACTGGTGTGTCTCATAAGTG | |
| | GGATACGAGCAGCGTG | | ACTTCTACCCGGGAGCCGTGACAG | |
| | | | TGGCCTGGAAGGCAGATAGCAGCC | |
| | | | CCGTCAAGGCGGGAGTGGAGACCA | |
| | | | CCACACCCTCCAAACAAAGCAACA | |
| | | | ACAAGTACGCGGCCAGCAGCTA | |
| S24-821 | CAGGTCACCTTGAGGGAGTCTGGTC | 1661 | GACATCCAGATGACCCAGTCTCCTT | 1751 |
| | CTGCGCTGGTGAAACCCACACAGAC | | CCACCCTGTCTGCATCTGTAGGAG | |
| | CCTCACACTGACCTGCACCTTCTCTG | | ACAGAGTCACCATCACTTGCCGGG | |
| | GGCTCTCACTCAGCAGTAGTGGAAT | | CCAGTCAGAGTATTAGTAGCTGGT | |
| | GTGTGTGAGCTGGATCCGTCAGCCC | | TGGCCTGGTATCAGCAGAAACCAG | |
| | CCAGGGAAGGCCCTGGAGTGGCTTG | | GGAAAGCCCCTAAGCTCCTGATCT | |
| | CACGCATTGATTGGGATGATGATAA | | ATAAGGCGTCTAGTTTAGAAAGTG | |
| | ATACTACAGCACATCTCTGAAGACC | | GGGTCCCATCAAGGTTCAGCGGCA | |
| | AGGCTCACCATCTCCAAGGACACCT | | GTGGATCTGGGACAGAATTCACTC | |
| | CCAAAAATCAGGTGGTCCTTACAAT | | TCACCATCAGCAGCCTGCAGCCTG | |
| | GACCAACATGGACCCTGTGGACACA | | ATGATTTTGCAACTTATTACTGCCA | |
| | GCCACGTATTACTGTGCACGGATAT | | ACAGTATAATAGTTATTCGTGGAC | |
| | GTACTATGGTTCGGGGACTCCATGA | | GTTCGGCCAAGGGACCAAGGTGGA | |
| | TGCTTTTGATATCTGGGGCCAAGGG | | AATCAAACGAACTGTGGCTGCACC | |
| | ACAATGGTCACCGTCTCTTCAGGGA | | ATCTGTCTTCATCTTCCCGCCATCT | |
| | GTGCATCCGCCCCAACCCTTTTCCCC | | GATGAGCAGTTGAAATCTGGAACT | |
| | CTCGTCTCCTGTGAGAATTCCCCGTC | | GCCTCTGTTGTGTGCCTGCTGAATA | |
| | GGATACGAGCAGCGTG | | ACTTCTATCCCAGAGAGGCCAAAG | |
| | | | TACAGTGGAAGGTGGATAACGC | |
| S144-67 | GAGGTGCAGCTGGTGCAGTCTGGAG | 1662 | CAGTCTGTGCTGACGCAGCCGCCC | 1752 |
| | CAGAGGTGAAAAAGCCCGGGGAGT | | TCAGTGTCTGGGGCCCCAGGGCAG | |
| | CTCTGAAGATCTCCTGTAAGGGTTC | | AGGGTCACCATCTCTTGCACTGGG | |
| | TGGATACAGCTTTACCACCTACTGG | | AGCAGGTCCAACATCGGGGCAGGT | |
| | ATCGCCTGGGTGCGCCAGATGCCCG | | TATGATGTACAGTGGTACCAGCAG | |
| | GGAAAGGCCTGGAGTGGGTGGGGA | | GTTCCAGGAACAGCCCCCAAACTC | |
| | TCATCTATCCTGATGACTCTGATACC | | CTCATCTCTGGTAACAGCAATCGG | |
| | AGATACAGCCCGTCCTTCCAAGGCC | | CCCTCAGGGGTCCCTGACCGATTCT | |
| | AGGTCACCATCTCAGCCGACAAGTC | | CTGGCTCCAAGTCTGGCACCTCAG | |
| | CATCGGTACCGCCTACCTGCAGTGG | | CCTCCCTGGCCATCACTGGGCTCCA | |
| | AGTAGCCTGAAGGCCTCGGACACCG | | GGCTGAGGATGAGGCTGATTATTA | |
| | CCATGTATTACTGTGCGAGGGGCCA | | CTGCCAGTCCTATGACAGCAGCCT | |
| | GTATTACGATTTTTGGAGCGGAGCC | | GAGTGGTCTGAGGGTATTCGGCGG | |
| | GGAGGTGTGGACGTCTGGGGCCAA | | AGGGACCAAGCTGACCGTCCTAGG | |
| | GGGACCACGGTCACCGTCTCCTCAG | | TCAGCCCAAGGCTGCCCCCTCGGT | |
| | CCTCCACCAAGGGCCCATCGGTCTT | | CACTCTGTTCCCGCCCTCCTCTGAG | |
| | CCCCCTGGCACCCTCCTCCAAGAGC | | GAGCTTCAAGCCAACAAGGCCACA | |
| | ACCTCTGGGGGCACAGCGGCCCTGG | | CTGGTGTGTCTCATAAGTGACTTCT | |
| | GCTGCCTGGTCAAGGACTACTTCCC | | ACCCGGGAGCCGTGACAGTGGCCT | |
| | CGAACCGGTGACGGTGTCGTGGAAC | | GGAAGGCAGATAGCAGCCCCGTCA | |
| | TCAGGCGCCCTGACCAGCGGCGTGC | | AGGCGGGAGTGGAGACCACCACAC | |
| | ACACCTTCCCGGCTGTCCTACAGTC | | CCTCCAAACAAAGCAACAACAAGT | |
| | CTCAGGA | | ACGCGGCCAGCAGCTATCTGAGCC | |
| | | | TGACGCCTGAGCAGTGGAAGTCCC | |
| | | | AC | |
| S144-69 | GAGGTGCAGCTGGTGCAGTCTGGAG | 1663 | GACATCCAGATGACCCAGTCTCCTT | 1753 |
| | CAGAGGTGAAAAAGCCCGGGGAGT | | CCACCCTGTCTGTATCTGTAGGAGA | |
| | CTCTGAAGATCTCCTGTAAGGGTTC | | CAGAGTCACCATCACTTGCCGGGC | |
| | TGGATACAGCTTTACCAGCTACTGG | | CAGTCAGAGTGTTAGTAGCTGGTT | |
| | ATCGGCTGGGTGCGCCAGATGCCCG | | GGCCTGGTATCAGCAGAAACCAGG | |
| | GGAAAGGCCTGGAGTGGATGGGGA | | GAAAGCCCCTAAGCTCCTGATCTA | |
| | TCATCTATCCTGGTGACTCTGATACC | | TGATGCCTCCAGTTTGGAAAGTGG | |
| | AGATACAGCCCGTCCTTCCAAGGCC | | GGTCCCATCAAGGTTCAGCGGCAG | |
| | AGGTCACCATCTCAGCCGACAAGTC | | TGGATCTGGGACAGAATTCACTCT | |
| | CATCACTACCGCCTACCTGCAGTGG | | CACCATTAGCAGCCTGCAGCCTGA | |
| | AGCAGCCTGAAGGCCTCGGACACCG | | TGATTTTGCAACTTATTACTGCCAA | |
| | CCATGTATTACTGTGCGAGGACCCA | | CAGTATAATAGTTTCTACACTTTTG | |
| | GACTACGAACTGGTTCGACTCCTGG | | GCCAGGGGACCAAGCTGGAGATCA | |
| | GGCCAGGGAACCCTGGTCACCGTCT | | AACGAACTGTGGCTGCACCATCTG | |
| | CCTCAGCCTCCACCAAGGGCCCATC | | TCTTCATCTTCCCGCCATCTGATGA | |
| | GGTCTTCCCCCTGGCACCCTCCTCCA | | GCAGTTGAAATCTGGAACTGCCTC | |
| | AGAGCACCTCTGGGGGCACAGCGG | | TGTTGTGTGCCTGCTGAATAACTTC | |
| | CCCTGGGCTGCCTGGTCAAGGACTA | | TATCCCAGAGAGGCCAAAGTACAG | |
| | CTTCCCCGAACCGGTGACGGTGTCG | | TGGAAGGTGGATAACGCCCTCCAA | |
| | TGGAACTCAGGCGCCCTGACCAGCG | | TCGGGTAACTCCCAGGAGAGTGTC | |
| | GCGTGCACACCTTCCCGGCTGTCCT | | ACAGAGCAGGACAGCAAGGACAG | |
| | ACAGTCCTCAGGA | | CACCTACAGCCTCAGCAGCACCCT | |
| | | | GACGCTGAGCAAAGCAGACTACGA | |
| | | | GAA | |
| S144-94 | CAGGTGCAGCTGGTGGAGTCTGGGG | 1664 | GATATTGTGATGACTCAGTCTCCAC | 1754 |
| | GAGGCGTGGTCCAGCCTGGGGGGTC | | TCTCCCTGCCCGTCACCCCTGGAGA | |
| | CCTGAGACTCTCCTGTGCAGCGTCT | | GCCGGCCTCCATCTCCTGCAGGTCT | |
| | GGATTCACCTTCAGTAGCTATGGCA | | AGTCAGAGCCTCCTGCATAGTAAT | |
| | TGCACTGGGTCCGCCAGGCTCCAGG | | GGATACAACTATTTGGATTGGTAC | |
| | CAAGGGGCTGGAGTGGGTGACATTT | | CTGCAGAAGCCAGGGCAGTCTCCA | |
| | ACACGGTATGATGGAAGTAATAAGT | | CAGCTCCTGATCTATTTGGGTTCTA | |
| | TCTATGCAGACTCCGTGAAGGGCCG | | ATCGGGCCTCCGGGGTCCCTGACA | |
| | ATTCTCCATCTCCAGAGACAATTCC | | GGTTCAGTGGCAGTGGATCAGGCA | |
| | AAGAACACGTTGTATCTGCAAATGA | | CAGATTTTACACTGAAAATCAGCA | |
| | ATAGTCTGAGAGCTGAGGACACGGC | | GAGTGGAGGCTGAGGATGTTGGGG | |
| | TGTATACTACTGCGCGAAAGAAAGT | | TTTATTACTGCATGCAAGCTCTACA | |
| | CGTGTGGCGTTTGGGGGAGCTATCG | | AACTCCTCAGTACACTTTTGGCCAG | |
| | CCATCTACTACTTCGGTATGGACGT | | GGGACCAAGCTGGAGATCAAACGA | |
| | CTGGGGCCAAGGGACCACGGTCACC | | ACTGTGGCTGCACCATCTGTCTTCA | |
| | GTCTCCTCAGCCTCCACCAAGGGCC | | TCTTCCCGCCATCTGATGAGCAGTT | |
| | CATCGGTCTTCCCCCTGGCGCCCTG | | GAAATCTGGAACTGCCTCTGTTGTG | |
| | CTCCAGGAGCACCTCTGGGGGCACA | | TGCCTGCTGAATAACTTCTATCCCA | |
| | GCGGCCCTGGGCTGCCTGGTCAAGG | | GAGAGGCCAAAGTACAGTGGAAG | |
| | ACTACTTCCCCGAACCGGTGACGGT | | GTGGATAACGCCCTCCAATCGGGT | |
| | GTCGTGGAACTCAGGCGCCCTGACC | | AACTCCCAGGAGAGTGTCACAGAG | |
| | AGCGGCGTGCACACCTTCCCGGCTG | | CAGGACAGCAAGGACAGCACCTAC | |
| | TCCTACAGTCCTCAGGA | | AGCCTCAGCAGCACCCTGACGCTG | |
| | | | AGCAAAGCAGACTACGAGAA | |
| S144-113 | GAGGTGCAGTTATTGGAGTCTGGGG | 1665 | GACATCCAGATGACCCAGTCTCCA | 1755 |
| | GAGGCTTGGTACAGCCTGGGGGGTC | | TCCTCCCTGTCTGCATCTGTAGGAG | |
| | CCTGAGACTCTCCTGTGCAGCCTCT | | ACAGAGTCACCATCACTTGCCGGG | |
| | GGATTCACCTTTAGCAACTATGCCA | | CAAGTCAGAGCATTAGCAACTATT | |
| | TGAGCTGGGTCCGCCAGGCTCCAGG | | TAAATTGGTATCAGCAGAAACCAG | |
| | GAAGGGGCTGGAGTGGGTCTCAGCT | | GGAAAGCCCCTGACCTCCTGATCT | |
| | ATTCGTAATAGTGGTAGTAGCACAT | | ATGCTGCATCCAGTTTGCAAAGTG | |
| | ACTATGCTGACTCCGTGAAGGGCCG | | GGGTCCCATTAAGGTTCAGTGGCA | |
| | GTTCACCATCTCCAGAGACAATTCC | | GTGGATCTGGGACAGATTTCACTCT | |
| | AAGAACACGCTGTATCTGCAAATGA | | CACCATCAGCAGTCTGCAACCTGA | |
| | ACAGCCTGAGAGCCGAGGACTCGG | | AGATTTTGCAACTTACTACTGTCAA | |
| | CCGTATATTACTGTGCGAAAGTAGG | | CAGACTTACAGTGCCCCCACTTTCG | |
| | GGGGACAGCAGCTGGTCATCCGTTT | | GCGGAGGGACCAAGGTGGAGATCA | |
| | TATGACTACTGGGGCCAGGGAACCC | | AACGAACTGTGGCTGCACCATCTG | |
| | TGGTCACCGTCTCCTCAGCCTCCAC | | TCTTCATCTTCCCGCCATCTGATGA | |
| | CAAGGGCCCATCGGTCTTCCCCCTG | | GCAGTTGAAATCTGGAACTGCCTC | |
| | GCACCCTCCTCCAAGAGCACCTCTG | | TGTTGTGTGCCTGCTGAATAACTTC | |
| | GGGGCACAGCGGCCCTGGGCTGCCT | | TATCCCAGAGAGGCCAAAGTACAG | |
| | GGTCAAGGACTACTTCCCCGAACCG | | TGGAAGGTGGATAACGCCCTCCAA | |
| | GTGACGGTGTCGTGGAACTCAGGCG | | TCGGGTAACTCCCAGGAGAGTGTC | |
| | CCCTGACCAGCGGCGTGCACACCTT | | ACAGAGCAGGACAGCAAGGACAG | |
| | CCCGGCTGTCCTACAGTCCTCAGGA | | CACCTACAGCCTCAGCAGCACCCT | |
| | CTC | | GACGCTGAGCAAAGCAGACTACGA | |
| | | | GAA | |
| S144-175 | CAGGTGCAGCTGGTGCAGTCTGGGG | 1666 | CAGTCTATGCTGACTCAGCCACCCT | 1756 |
| | CTGAGGTGAAGAAGCCTGGGGCCTC | | CAGCGTCTGGGACCCCCGGGCAGA | |
| | AGTGAAGGTCTCCTGCAAGGCTTCT | | GGGTCACCATCTCTTGTTCTGGAAG | |
| | GGATACACCTTCACCGGCTACTATA | | CAGCTCCAACATCGGAAGTAATTA | |
| | TGCACTGGGTGCGACAGGCCCCTGG | | TGTATACTGGTACCAGCAGCTCCC | |
| | ACAAGGTCTTGAGTGGATGGGACGG | | AGGAACGGCCCCCAAACTCCTCAT | |
| | ATCAACCCTAACAGTGGTGGCACAA | | CTATAGGAATAATCAGCGGCCCTC | |
| | ACTTTGCACAGAGGTTTCAGGGCAG | | AGGGGTCCCTGACCGATTCTCTGG | |
| | GGTCTCCATGACCAGGGACACCTCC | | CTCCAAGTCTGGCACCTCAGCCTCC | |
| | ATCAGCACAGCCTACATGGAACTGA | | CTGGCCATCAGTGGGCTCCGGTCC | |
| | GCAGCCTGAGATCTGACGACACGGC | | GAGGATGAGGCTGATTATTACTGT | |
| | CGTATATTACTGTGCGAGAGGCGCA | | GCAGCATGGGATGACAGACGTTGG | |
| | AAATTCGAGCACCTCCCTTTTGATA | | GTGTTCGGCGGAGGGACCAAGCTG | |
| | TCTGGGGCCAAGGGACAATGGTCAC | | ACCGTCCTAGGTCAGCCCAAGGCT | |
| | CGTCTCTTCAGCCTCCACCAAGGGC | | GCCCCCTCGGTCACTCTGTTCCCAC | |
| | CCATCGGTCTTCCCCCTGGCACCCTC | | CCTCCTCTGAGGAGCTTCAAGCCA | |
| | CTCCAAGAGCACCTCTGGGGGCACA | | ACAAGGCCACACTGGTGTGTCTCA | |
| | GCGGCCCTGGGCTGCCTGGTCAAGG | | TAAGTGACTTCTACCCGGGAGCCG | |
| | ACTACTTCCCCGAACCGGTGACGGT | | TGACAGTGGCCTGGAAGGCAGATA | |
| | GTCGTGGAACTCAGGCGCCCTGACC | | GCAGCCCCGTCAAGGCGGGAGTGG | |
| | AGCGGCGTGCACACCTTCCCGGCTG | | AGACCACCACACCCTCCAAACAAA | |
| | TCCTACAGTCCTCAGGA | | GCAACAACAAGTACGCGGCCAGCA | |
| | | | GCTA | |
| S144-208 | CAGGTGCAACTGGTGCAGTCTGGGG | 1667 | CAGTCTGCCCTGACTCAGCCTCGCT | 1757 |
| | CTGAGGTGAAGAAGCCTGGGGCCTC | | CAGTGTCCGGGTCTCCTGGACAGT | |
| | AGTGAAGGTCTCCTGCAAGTCTTCT | | CAGTCACTATCTCCTGCACTGGAAC | |
| | GGATACACCTTCACCGGCTACTATA | | CAGCAGTGATGTTGGTGGTTATAA | |
| | TGCACTGGGTGCGACAGGCCCCTGG | | GTATGTCTCCTGGTACCAACAGCA | |
| | ACAAGGGCTTGAGTGGATGGGACG | | CCCAGGCAAAGCCCCCAAACTCAT | |
| | GATCAACCCTAATAGTGGTGGCACA | | GATTTATGACGTCAGTAAGCGGCC | |
| | AACTATGCACAGAAGTTTCAGGGCA | | CTCAGGGGTCCCTGATCGCTTCTCT | |
| | GGGTCACCATGACCAGGGACACGTC | | GGCTCCAAGTCTGGCAACACGGCC | |
| | CATCAGCACAGCCTACATGGAACTG | | TCCCTGACCATCTCTGGGCTCCAGG | |
| | AGCAGGCTGAGATCTGACGACACG | | CTGAGGATGAGGGTGATTATTACT | |
| | GCCGTATATTACTGTGCGAGAGGGG | | GCTGCTCATATGCAGGCACCTACA | |
| | CCCGAGGTGGCGCGGGGTGCAGTG | | GTTTGGTATTCGGCGGAGGGACCA | |
| | GCTGGTCATGTTTTGACTTCTGGGG | | AGGTGACCGTGACCGTCCTAGGTC | |
| | CCAGGGAACCCTGGTCACCGTCTCC | | AGCCCAAGGCTGCCCCCTCGGTCA | |
| | TCAGCCTCCACCAAGGGCCCATCGG | | CTCTGTTCCCGCCCTCCTCTGAGGA | |
| | TCTTCCCCCTGGCACCCTCCTCCAAG | | GCTTCAAGCCAACAAGGCCACACT | |
| | AGCACCTCTGGGGGCACAGCGGCCC | | GGTGTGTCTCATAAGTGACTTCTAC | |
| | TGGGCTGCCTGGTCAAGGACTACTT | | CCGGGAGCCGTGACAGTGGCCTGG | |
| | CCCCGAACCGGTGACGGTGTCGTGG | | AAGGCAGATAGCAGCCCCGTCAAG | |
| | AACTCAGGCGCCCTGACCAGCGGCG | | GCGGGAGTGGAGACCACCACACCC | |
| | TGCACACCTTCCCGGCTGTCCTACA | | TCCAAACAAAGCAACAACAAGTAC | |
| | GTCCTCAGGA | | GCGGCCAGCAGCTATCTGAGCCTG | |
| | | | ACGCCTGAGCAGTGGAAGTCCCAC | |
| | | | A | |
| S144-339 | GAGGTGCAGCTGGTGGAGTCTGGGG | 1668 | GAAATTGTGTTGACGCAGTCTCCA | 1758 |
| | GAGGCCTGGTCAAGCCGGGGGGGT | | GGCACCCTGTCTTTGTCTCCAGGGG | |
| | CCCTGAGACTCTCCTGTGCAGCCTC | | AAAGAGCCACCCTCTCCTGCAGGG | |
| | TGGATTCACCTTCAGTGACTATACC | | CCAGTCAGAGTCTTAGCAGCAGCT | |
| | ATGAACTGGGTCCGACAGGCTCCAG | | ACTTAGCCTGGTACCAGCAGAAAC | |
| | GGAAGGGACTGGAGTGGGTCTCATC | | CTGGCCAGTCTCCCAGGCTCCTCAT | |
| | CATTACTAGAAGTAGTACTTACATC | | TTATGGTGCATCCAGCAGGGCCAC | |
| | TACTACGCAGACTCAGTGAAGGGCC | | TGGCATCCCAGACAGGTTCAGTGG | |
| | GATTCACCATCTCCAGAGACAACGC | | CAGTGGGTCTGGGACAGACTTCAC | |
| | CAAGAACTCACTGTATCTGCAAATG | | TCTCACCATCAACAGACTGGAGCC | |
| | AACAGCCTGAGAGCCGAGGACACG | | TGAAGATTTTGCAGTATATTACTGT | |
| | GCTGTCTATTACTGTGCGAGAGACC | | CAGCAGTATCGTACCTCACCTCGA | |
| | CCTATTACGATATTTTGACTGGTTAT | | GGCACTTTCGGCGGAGGGACCAAG | |
| | TGGAACTACTGGGGCCAGGGAACCC | | GTGGAGATCAAACGAACTGTGGCT | |
| | TGGTCACCGTCTCCTCAGCCTCCAC | | GCACCATCTGTCTTCATCTTCCCGC | |
| | CAAGGGCCCATCGGTCTTCCCCCTG | | CATCTGATGAGCAGTTGAAATCTG | |
| | GCACCCTCCTCCAAGAGCACCTCTG | | GAACTGCCTCTGTTGTGTGCCTGCT | |
| | GGGGCACAGCGGCCCTGGGCTGCCT | | GAATAACTTCTATCCCAGAGAGGC | |
| | GGTCAAGGACTACTTCCCCGAACCG | | CAAAGTACAGTGGAAGGTGGATAA | |
| | GTGACGGTGTCGTGGAACTCAGGCG | | CGCCCTCCAATCGGGTAACTCCCA | |
| | CCCTGACCAGCGGCGTGCACACCTT | | GGAGAGTGTCACAGAGCAGGACAG | |
| | CCCGGCTGTCCTACAGTCCTCAGGA | | CAAGGACAGCACCTACAGCCTCAG | |
| | | | CAGCACCCTGACGCTGAGCAAAGC | |
| | | | AGACTACGAGAA | |
| S144-359 | GAGGTGCAGCTGGTGGAGTCTGGGG | 1669 | GACATCCAGATGACCCAGTCTCCA | 1759 |
| | GAGGCTTGGTACAGCCTGGGGGGTC | | TCCTCCCTGTCTGCATCTGTAGGAG | |
| | CCTGAGACTCTCCTGTGCAGCCTCT | | ACAGAGTCACCATCACTTGCCGGG | |
| | GGATTCACCTTTAGCAGCTATGCCA | | CAAGTCAGAGCATTAGCAGCTATT | |
| | TGAGCTGGGTCCGCCAGGCTCCAGG | | TAAATTGGTATCAGCAGAAACCAG | |
| | GAAGGGGCTGGAGTGGGTCTCATCT | | GGAAAGCCCCTAAGCTCCTGATCT | |
| | ATTAGAGGTAGTGGTGGTAGCACAT | | ATGCTGCATCCAGTTTGCAAAGTG | |
| | ACTACGCAGACTCCGTGAAGGGCCG | | GGGTCCCATCAAGGTTCAGTGGCA | |
| | GTTCACCATCTCCAGAGACAACTCC | | GTGGATCTGGGACAGATTTCACTCT | |
| | AAGTACACGTTGTATCTGCAAATGA | | CACCATCAGCAGTCTGCAACCTGA | |
| | ACAGCCTGAGAGCCGAGGACACGG | | AGATTTTGCAATTTACTACTGTCAA | |
| | CCGTATATTACTGTGCGAAAATAAC | | CAGACTTCCCGTACCCCGCTCACTT | |
| | TGGAGCCGTCGGGGGGGAGAACTG | | TCGGCGGAGGGACCAAGGTGGAGG | |
| | GTTCGACCCCTGGGGCCAGGGAACC | | TCAAACGAACTGTGGCTGCACCAT | |
| | CTGGTCACCGTCTCCTCAGCCTCCA | | CTGTCTTCATCTTCCCGCCATCTGA | |
| | CCAAGGGCCCATCGGTCTTCCCCCT | | TGAGCAGTTGAAATCTGGAACTGC | |
| | GGCGCCCTGCTCCAGGAGCACCTCT | | CTCTGTTGTGTGCCTGCTGAATAAC | |
| | GGGGGCACAGCGGCCCTGGGCTGCC | | TTCTATCCCAGAGAGGCCAAAGTA | |
| | TGGTCAAGGACTACTTCCCCGAACC | | CAGTGGAAGGTGGATAACGCCCTC | |
| | GGTGACGGTGTCGTGGAACTCAGGC | | CAATCGGGTAACTCCCAGGAGAGT | |
| | GCCCTGACCAGCGGCGTGCACACCT | | GTCACAGAGCAGGACAGCAAGGAC | |
| | TCCCGGCTGTCCTACAGTCCTCAGG | | AGCACCTACAGCCTCAGCAGCACC | |
| | A | | CTGACGCTGAGCAAAGCAGACTAC | |
| | | | GAGAA | |
| S144-460 | GAGGTGCGCCTGGTGCAGTCTGGGG | 1670 | GACATCCAGATGACCCAGTCTCCA | 1760 |
| | GAGGCTTGGTAAAGCCCGGGGGGTC | | TCTGCCATGTCTGCATCTGTAGGAG | |
| | CCTGAGACTCTCCTGTGCAGCCTCT | | ACAGAGTCACCATCACTTGTCGGG | |
| | GGATTCACCTTCAGCACCGCCTGGG | | CGAGTCAGGACATTAACACCTTTTT | |
| | TGAGGTGGGTCCGCCAGGCTCCAGG | | AACGTGGTTTCAGCAGAAACCAGG | |
| | GAAGGGGCTGGAGTGCGTTGGCCG | | AAAAGTCCCTCAGCGCCTGATCTTT | |
| | AATCAAAAGTAAAAATGACGGTGA | | GCTGCATATCGTTTGCAAAGTGGG | |
| | CAGAGCAGAGTACGCTGCACCCGCG | | GTCCCTTCAAGGTTCAGTGGCAGT | |
| | AGAGGCAGATTCATCATCTCAAGAG | | GGATCTGGGACAGAATTCACTCTC | |
| | ATGATGCAGAAAACATTCTGTATTT | | ACAATCAACAGCCTGCAGCCTGAA | |
| | ACAAATGAACAACCTGAAAACCGA | | GATGTTGCGACTTATTATTGTCTAC | |
| | GGACACAGCCTTTTATTACTGTACC | | ACCATAAAACTTATCCGTACACTTT | |
| | ACGGATCAAGGAAATAGTAGTGCCT | | TGGCCAGGGGACCAAACTGGAGAT | |
| | TCTACAGTGCTGACTATTGGGGCCA | | CAAACGAACTGTGGCTGCACCATC | |
| | GGGAACCCTGGTCACCGTCTCCTCA | | TGTCTTCATCTTCCCGCCATCTGAT | |
| | GCATCCCCGACCAGCCCCAAGGTCT | | GAGCAGTTGAAATCTGGAACTGCC | |
| | TCCCGCTGAGCCTCGACAGCACCCC | | TCTGTTGTGTGCCTGCTGAATAACT | |
| | CCAAGATGGGAACGTGGTCGTCGCA | | TCTATCCCAGAGAGGCCAAAGTAC | |
| | TGCCTGGTCCAGGGCTTCTTCCCCC | | AGTGGAAGGTGGATAACGCCCTCC | |
| | AGGAGCCACTCAGTGTGACCTGGAG | | AATCGGGTAACTCCCAGGAGAGTG | |
| | CGAAAGCGGACAGAACGTGACCGC | | TCACAGAGCAGGACAGCAAGGACA | |
| | CAGAAACTTCCC | | GCACCTACAGCCTCAGCAGCACCC | |
| | | | TGACGCTGAGCAAAGCAGACTACG | |
| | | | AGAA | |
| S144-466 | GAGGTGCAGCTGGTGCAGTCTGGAG | 1671 | GACATCCAGATGACCCAGTCTCCTT | 1761 |
| | CAGAGGTGAAAAAGCCCGGGGAGT | | CCACCCTGTCTGCATCTGTGGGAG | |
| | CTCTGAAGATCTCCTGTAAGGGTTC | | ACAGAGTCACCATCACTTGCCGGG | |
| | TGGATACAGGTTTACCAGATACTGG | | CCAGTCAGAGTATTACTAGTTGGTT | |
| | ATCGGCTGGGTGCGCCAGATGCCCG | | GGCCTGGTATCAGCAGAAATCAGG | |
| | GGAAAGGCCTGGAGTGGATGGGGA | | GAAAGCCCCTAAACTCCTGATCTA | |
| | TCATCTATCTTGGTGACTCTGAAAC | | TGATGCCTCCAGTTTGGAAAGTGG | |
| | CAGATACAGTCCGTCCTTCCAAGGC | | GGTCCCATCAAGGTTCAGCGGCAG | |
| | CAGGTCACCATCTCAGCCGACAACT | | TGGATCTGGGACAGAATTCACTCT | |
| | CCATCAGCACCGCCTACCTGCAGTG | | CACCATCAGCAGCCTGCAGCCTGA | |
| | GAGCAGCCTGAAGGCCTCGGACACC | | TGATTTTGCAACTTATTACTGCCAA | |
| | GCCATGTATTACTGTGCGAGAAGTT | | CAGTATAATAGTTATCCTTGGACGT | |
| | CCAATTGGAATTACGGTGACTACTG | | TCGGCCAAGGGACCAAGGTGGAAA | |
| | GGGCCAGGGAACCCTGGTCACCGTC | | TCAAACGAACTGTGGCTGCACCAT | |
| | TCCTCAGCTTCCACCAAGGGCCCAT | | CTGTCTTCATCTTCCCGCCATCTGA | |
| | CGGTCTTCCCCCTGGCGCCCTGCTCC | | TGAGCAGTTGAAATCTGGAACTGC | |
| | AGGAGCACCTCTGGGGGCACAGCG | | CTCTGTTGTGTGCCTGCTGAATAAC | |
| | GCCCTGGGCTGCCTGGTCAAGGACT | | TTCTATCCCAGAGAGGCCAAAGTA | |
| | ACTTCCCCGAACCGGTGACGGTGTC | | CAGTGGAAGGTGGATAACGCCCTC | |
| | GTGGAACTCAGGCGCCCTGACCAGC | | CAATCGGGTAACTCCCAGGAGAGT | |
| | GGCGTGCACACCTTCCCGGCTGTCC | | GTCACAGAGCAGGACAGCAAGGAC | |
| | TACAGTCCTCAGGA | | AGCACCTACAGCCTCAGCAGCACC | |
| | | | CTGACGCTGAGCAAAGCAGACTAC | |
| | | | GAGAA | |
| S144-469 | CAGGTGCAGCTGCAGGAGTCGGGCC | 1672 | GATATTGTGATGACTCAGTCTCCAC | 1762 |
| | CAGGACTGGTGAAGCCTTCGGAGAC | | TCTCCCTGCCCGTCACCCCTGGAGA | |
| | CCTGTCCCTCACCTGCACTGTCTCTG | | GCCGGCCTCCATCTCCTGCAGGTCT | |
| | GTGGCTCCATCAGTAGTGACTACTG | | AGTCAGAGCCTCCTGCATAGTAAT | |
| | GAGCTGGATCCGGCAGCCCCCAGGG | | GGATACAACTATTTGGATTGGTAC | |
| | AAGGGACTGGAGTGGATTGGATATA | | CTGCAGAAGCCAGGGCAGTCTCCA | |
| | TGTATTACAGTGGGAGCACCAACTA | | CAGCTCCTGATCTATTTGGGTTCTA | |
| | CAACCCCTCCCTCAAGAGTCGAGTC | | ATCGGGCCTCCGGGGTCCCTGACA | |
| | ACCATATCAGTAGACACGTCCAAGA | | GGTTCAGTGGCAGTGCATCAGGCA | |
| | ACCAGTTCTCCCTGAAGCTGAGCTC | | CAGATTTTACACTGAAAATCAGCA | |
| | TGTGACCGCTGCGGACACGGCCGTG | | GAGTGGAGGCTGAGGATGTTGGGG | |
| | TATTACTGTGCGAGATGGGATAGGG | | TTTATTACTGCATGCAAGCTCTACA | |
| | GAAGCAGGCCTCACTACTACTACTA | | AGCTTTCACTTTCGGCCCTGGGACC | |
| | TGGTATGGACGTCTGGGGCCAAGGG | | AAAGTGGATATCAAACGAACTGTG | |
| | ACCACGGTCACCGTCTCCTCAGCCT | | GCTGCACCATCTGTCTTCATCTTCC | |
| | CCACCAAGGGCCCATCGGTCTTCCC | | CGCCATCTGATGAGCAGTTGAAAT | |
| | CCTGGCACCCTCCTCCAAGAGCACC | | CTGGAACTGCCTCTGTTGTGTGCCT | |
| | TCTGGGGGCACAGCGGCCCTGGGCT | | GCTGAATAACTTCTATCCCAGAGA | |
| | GCCTGGTCAAGGACTACTTCCCCGA | | GGCCAAAGTACAGTGGAAGGTGGA | |
| | ACCGGTGACGGTGTCGTGGAACTCA | | TAACGCCCTCCAATCGGGTAACTC | |
| | GGCGCCCTGACCAGCGGCGTGCACA | | CCAGGAGAGTGTCACAGAGCAGGA | |
| | CCTTCCCGGCTGTCCTACAGTCCTCA | | CAGCAAGGACAGCACCTACAGCCT | |
| | GGA | | CAGCAGCACCCTGACGCTGAGCAA | |
| | | | AGCAGACTACGAGA | |
| S144-509 | GAGGTGCAGCTGGTGCAGTCTGGAG | 1673 | GACATCCAGATGACCCAGTCTCCTT | 1763 |
| | CAGAGGTGAAAAAGCCCGGGGAGT | | CCACCCTGTCTGCATCTGTAGGAG | |
| | CTCTGAAGATCTCCTGTAAGGGTTC | | ACAGAGTCACCATCACTTGCCGGG | |
| | TGCATACACCTTTACCACCTACTGG | | CCAGTCAGAGTATTAGTAGCTGGT | |
| | ATCGGCTGGGTGCGCCAGATGCCCG | | TGGCCTGGTATCAGCAGAAACCAG | |
| | GGAAAGGCCTGGAGTGGATGGGGA | | GGAAAGCCCCTAACCTCCTGATCT | |
| | TCATCTATCCTGGTGACTCTGATACC | | ATGATGCCTCCAGTTTGGAAAGTG | |
| | AGATACAGCCCGTCCTTCCAAGGCC | | GGGTCCCATCAAGGTTCAGCGGCA | |
| | AGGTCACCATCTCAGCCGACAAGTC | | GTGGATCTGGGACAGAATTCACTC | |
| | CATCAGCACCGCCTACCTGCAGTGG | | TCACCATCAGCAGCCTGCAGCCTG | |
| | AGCAGCCTGAAGGCCTCGGACACCG | | ATGATTTTGCAACTTATTACTGCCA | |
| | CCATGTATTACTGTGCGAGATTATT | | ACAGTATAATAGTTATCCGTGGAC | |
| | ATTGGTGGCTGGTCCCTTTGACTACT | | GTTCGGCCAAGGGACCAAGGTGGA | |
| | GGGGCCAGGGAACCCTGGTCACCGT | | AATCAAACGAACTGTGGCTGCACC | |
| | CTCCTCAGCCTCCACCAAGGGCCCA | | ATCTGTCTTCATCTTCCCGCCATCT | |
| | TCGGTCTTCCCCCTGGCACCCTCCTC | | GATGAGCAGTTGAAATCTGGAACT | |
| | CAAGAGCACCTCTGGGGGCACAGC | | GCCTCTGTTGTGTGCCTGCTGAATA | |
| | GGCCCTGGGCTGCCTGGTCAAGGAC | | ACTTCTATCCCAGAGAGGCCAAAG | |
| | TACTTCCCCGAACCGGTGACGGTGT | | TACAGTGGAAGGTGGATAACGC | |
| | CGTGGAACTCAGGCGCCCTGACCAG | | | |
| | CGGCGTGCACACCTTCCCGGCTGTC | | | |
| | CTACAGTCCTCAGGACTCTACTCCC | | | |
| | TCAGCAGCGTGGTGACCGTGCCCTC | | | |
| | CAGCAGCTTGGGCACCCAGACCTAC | | | |
| | ATCTGCAACGTGAATCACAAGCCCA | | | |
| | GCAACACCAAGGTGGACA | | | |
| S144-516 | CAGGTGCAGCTGCTGCAGTCTGGGG | 1674 | CAGTCTGTGCTGACGCAGCCGCCC | 1764 |
| | CTGAAGTGAAGAAGCCTGGGGCCTC | | TCAGTGTCTGAGGCCCCAGGGCAG | |
| | AGTGAAGGTCTCCTGCAAGGCTTCT | | AGGGTCACCATCTCCTGCACTGGG | |
| | GGATACACCTTCACCGGCTACTATA | | AGCAGCTCCAACATCGGGGCAGGT | |
| | TGCACTGGGTGCGACAGGCCCCTGG | | TATGATGTACACTGGTACCAGCAG | |
| | ACAAGGGCTTGAGTGGATGGGACG | | CTTCCAGGAACAGCCCCCAAACTG | |
| | GATCAACCCTAACAGTGGTGGCACA | | CTCATCTATGGTAACATTAATCGGC | |
| | AATTATGCACAGAAGTTTCAGGGCA | | CCTCAGGGGTCCCTGACCGATTCTC | |
| | GGGTCACCATGACCAGGGACACGTC | | TGGCTCCAAGTCTGGCACCTCAGC | |
| | CATCAGCACAGCCTACATGGAGCTG | | CTCCCTGGCCATCACTGGGCTCCAG | |
| | AGCAGGCTGACATCTGACGACACGG | | GCTGAGGATGAGGCTGATTATTAC | |
| | CCGTGTATTACTGTGCGACCAAAAC | | TGCCAGTCCTATGACAACAGCCTG | |
| | TGGAATTGATCGCTACTACTACTAC | | AATGGTTCGGTGTTCGGCGGAGGG | |
| | TACATGGACGTCTGGGGCAAAGGG | | ACCAAACTGACCGTCCTACGTCAG | |
| | ACCACGGTCACCGTCTCCTCAGCCT | | CCCAAGGCTGCCCCCTCGGTCACTC | |
| | CCACCAAGGGCCCATCGGTCTTCCC | | TGTTCCCACCCTCCTCTGAGGAGCT | |
| | CCTGGCACCCTCCTCCAAGAGCACC | | TCAAGCCAACAAGGCCACACTGGT | |
| | TCTGGGGGCACAGCGGCCCTGGGCT | | GTGTCTCATAAGTGACTTCTACCCG | |
| | GCCTGGTCAAGGACTACTTCCCCGA | | GGAGCCGTGACAGTGGCCTGGAAG | |
| | ACCGGTGACGGTGTCGTGGAACTCA | | GCAGATAGCAGCCCCGTCAAGGCG | |
| | GGCGCCCTGACCAGCGGCGTGCACA | | GGAGTGGAGACCACCACACCCTCC | |
| | CCTTCCCGGCTGTCCTACAGTCCTCA | | AAACAAAGCAACAACAAGTACGCG | |
| | GGA | | GCCAGCAGCTA | |
| S144-568 | CAGGTGCAGCTGCAGGAGTCGGGCC | 1675 | GAAATTGTGTTGACGCAGTCTCCA | 1765 |
| | CAGGACTGGTGAAGCCTTCGGAGAC | | GGCACCCTGTCTTTGTCTCCAGGGG | |
| | CCTGTCCCTCACCTGCAGTGTCTCTG | | AAAGAGCCACCCTCTCATGTAGGG | |
| | GTGGCTCCATCAGTGATTACTACTG | | CCAGTCAGAGTGTTAGCAGCAACT | |
| | GAGCTGGATCCGGCAGCCCCCTGGG | | TCCTAGCCTGGTACCAGCAGAAAC | |
| | AAGGGACTGGAGTGGATTGGATATA | | CTGGCCAGCCTCCCAGGCTCCTCAT | |
| | TCTATAACAGTGGGAGTACCAACTA | | CTATGGTGCATCCGTCAGGGCCAC | |
| | CAACCCCTCCCTCAAGAGTCGAGTC | | TGGCATCCCAGACAGGTTCAGTGG | |
| | ACCATATCAGCAGACCCGTCCAAGA | | CAGTGGGTCTGGGACAGACTTCAC | |
| | ACCAGTTCTCCCTGAAGTTGAGCTC | | TCTCACCATCACCAGACTGGAGCC | |
| | TGTGACCGCCGCAGACACGGCCGTA | | TGAAGATTTTGCAGTATATTACTGT | |
| | TATTACTGTGCGAGACCTCACGGCG | | CAGCAGTATGGTAGCTTACCTCGG | |
| | GTGACTACGCTTTTGATATTTGGGG | | ACGTTCGGCCAAGGGACCAAGGTG | |
| | CCAAGGGACAATGGTCACCGTCTCT | | GAAATCAAACGAACTGTGGCTGCA | |
| | TCAGCATCCCCGACCAGCCCCAAGG | | CCATCTGTCTTCATCTTCCCGCCAT | |
| | TCTTCCCGCTGAGCCTCGACAGCAC | | CTGATGAGCAGTTGAAATCTGGAA | |
| | CCCCCAAGATGGGAACGTGGTCGTC | | CTGCCTCTGTTGTGTGCCTGCTGAA | |
| | GCATGCCTGGTCCAGGGCTTCTTCC | | TAACTTCTATCCCAGAGAGGCCAA | |
| | CCCAGGAGCCACTCAGTGTGACCTG | | AGTACAGTGGAAGGTGGATAACGC | |
| | GAGCGAAAGCGGACAGAACGTGAC | | CCTCCAATCGGGTAACTCCCAGGA | |
| | CGCCAGAAACTTCCC | | GAGTGTCACAGAGCAGGACAGCAA | |
| | | | GGACAGCACCTACAGCCTCAGCAG | |
| | | | CACCCTGACGCTGAGCAAAGCAGA | |
| | | | CTACGAGA | |
| S144-576 | CAGGTCCAGCTGGTGCAATCTGGGG | 1676 | CATCCAGATGACCCAGTCTCCTTCC | 1766 |
| | CTGAGGTGATGAAGCCTGGGTCCTC | | ACCCTGTCTGCATCTGTAGGAGAC | |
| | GGTGAAGGTCTCCTGCAAGGCTTCT | | AGAGTCACCATCACTTGCCGGGCC | |
| | GGAGGCACCTTCAGCAGCTATAGTA | | AGTCAGAGTATTAGTAGCTGGTTG | |
| | TCACCTGGGTGCGACAGGCCCCTGG | | GCCTGGTATCAGCAGAAACCAGGG | |
| | ACAAGGGCTTGAGTGGATGGGAAG | | AAAGCCCCTAAGCTCCTGATCTAT | |
| | GATCATCCCTATCCTTGGTATAGCA | | GATGCCTCCAGTTTGCAAAGTGGG | |
| | AACTACGCACAGAAGTTCCAGGGCA | | GTCCCATCAAGGTTCAGCGGCAGT | |
| | GAGTCACGATTACCGCGGACAAATC | | GGATCTGGGACAGAATTCACTCTC | |
| | CACGAGCACAGCCTACATGGAGCTG | | ACCATCAGCAGCCTGCAGCCTGAT | |
| | AGCAGCCTGAGATCTGAGGACACG | | GATTTTGCAACTTATTACTGCCAAC | |
| | GCCGTGTATTACTGTGCGAGAGGGT | | AGTATAATAGTTATTCTCCGATCAC | |
| | ATAGTGGGAGCCCCTCGAATTTAGA | | CTTCGGCCAAGGGACACGACTCGA | |
| | CGGTATGGACGTCTGGGGCCAAGGG | | GATTAAACGAACTGTGGCTGCACC | |
| | ACCACGGTCACCGTCTCCTCAGCCT | | ATCTGTCTTCATCTTCCCGCCATCT | |
| | CCACCAAGGGCCCATCGGTCTTCCC | | GATGAGCAGTTGAAATCTGGAACT | |
| | CCTGGCACCCTCCTCCAAGAGCACC | | GCCTCTGTTGTGTGCCTGCTGAATA | |
| | TCTGGGGGCACAGCGGCCCTGGGCT | | ACTTCTATCCCAGAGAGGCCAAAG | |
| | GCCTGGTCAAGGACTACTTCCCCGA | | TACAGTGGAAGGTGGATAACGCCC | |
| | ACCGGTGACGGTGTCGTGGAACTCA | | TCCAATCGGGTAACTCCCAGGAGA | |
| | GGCGCCCTGACCAGCGGCGTGCACA | | GTGTCACAGAGCAGGACAGCAAGG | |
| | CCTTCCCGGCTGTCCTACAGTCCTCA | | ACAGCACCTACAGCCTCAGCAGCA | |
| | GGA | | CCCTGACGCTGAGCAAAGCAGACT | |
| | | | ACGAGAA | |
| S144-588 | CAGCTGCAGCTGCAGGAGTCGGGCC | 1677 | TCCTATGAGCTGACTCAGCCACCCT | 1767 |
| | CAGGACTGGTGAAGCCTTCGGAGAC | | CAGTGTCCGTGTCCCCAGGACAGA | |
| | CCTGTCCCTCACCTGCACTGTCTCTG | | CAGCCAGCATCACCTGCTCTGGAG | |
| | GTGGCTCCATCAGCAGTAGTAGTTA | | ATAAATTGGGGGATAAATATGCTT | |
| | CTACTGGGGCTGGATCCGCCAGCCC | | GCTGGTATCAGCAAAAGCCAGGCC | |
| | CCAGGGAAGGGGCTGGAGTGGATT | | AGTCCCCTGTGCTGGTCATCTATCA | |
| | GGGAGTATCTATTATAGTGGGAGCA | | AGATACCAAGCGGCCCTCAGGGAT | |
| | CCTACTACAACCCGTCCCTCAAGAG | | CCCTGAGCGATTCTCTGGCTCCAAC | |
| | TCGATTCACCATATCCGTAGACACG | | TCTGGGAACACAGCCACTCTGACC | |
| | TCCAAGAACCAGTTCTCCCTGAAGC | | ATCAGCGGGACCCAGGCTATGGAT | |
| | TGAGCTCTGTGACCGCCGCAGACAC | | GAGGCTGACTATTACTGTCAGGCG | |
| | GGCTGTGTATTACTGTGCGGCCTAT | | TGGGACAGTAGCACTGTGTTATTC | |
| | CAGAGGAAACTAGGATATTGTCGTG | | GGCGGAGGGACCAAGCTGACCGTC | |
| | GTAATAGCTGCTTTTCCTGCTTCGAC | | CTAGGTCAGCCCAAGGCTGCCCCC | |
| | CCCTGGGGCCAGGGAACCCTGGTCA | | TCGGTCACTCTGTTCCCGCCCTCCT | |
| | CCGTCTCCTCAGCCTCCACCAAGGG | | CTGAGGAGCTTCAAGCCAACAAGG | |
| | CCCATCGGTCTTCCCCCTGGCACCCT | | CCACACTGGTGTGTCTCATAAGTG | |
| | CCTCCAAGAGCACCTCTGGGGGCAC | | ACTTCTACCCGGGAGCCGTGACAG | |
| | AGCGGCCCTGGGCTGCCTGGTCAAG | | TGGCCTGGAAGGCAGATAGCAGCC | |
| | GACTACTTCCCCGAACCGGTGACGG | | CCGTCAAGGCGGGAGTGGAGACCA | |
| | TGTCGTGGAACTCAGGCGCCCTGAC | | CCACACCCTCCAAACAAAGCAACA | |
| | CAGCGGCGTGCACACCTTCCCGGCT | | ACAAGTACGCGGCCAGCAGCTATC | |
| | GTCCTACAGTCCTCAGGA | | TGAGCCTGACGCCTGAGCAGTGGA | |
| | | | AGTCCCACA | |
| S144-628 | GAGGTGCACCTGGTGCAGTCTGGAG | 1678 | CAGTCTGTGCTGACGCAGCCGCCC | 1768 |
| | CAGAGGTGAAACAGCCCGGGGAGT | | TCAATGTCTGGGGCCCCAGGGCAG | |
| | CTCTGAAGATCTCCTGTAAGGGTTC | | AGGGTCACCATCTCCTGCACTGGG | |
| | TGGATACAACTTTGCCACCTACTGG | | AGCAGCTCCAACATCGGGGCAGGT | |
| | ATCGCCTGGGTGCGCCAGATGCCCG | | TATGATGTACACTGGTACCAGCAG | |
| | GGAAAGGCCTGGAGTGGATGGGGA | | CTTCCAGGAGCAGCCCCCAAACTC | |
| | TCATCTATCCTGGTGACTCTGATACC | | CTCATCTATGGTGACACCAGTCGG | |
| | AGATACAGCCCGTCCTTCCAAGGCC | | CCCTCAGGGGTCCCTGACCGATTCT | |
| | AGGTCATCATCTCAGCCGACAAGTC | | CTGGCTCCAAGTCTGACACCTCAG | |
| | CATCGGCACCGCCTTCCTGCAGTGG | | CCTCCCTGGCCATCACTGGGCTCCA | |
| | AGCAGCCTGAAGGCCTCGGACACCG | | GGCTGAGGATGAGGCTGATTATTA | |
| | CCATGTATTACTGTGCGAGGCGGGG | | CTGCCAGTCCTTTGACAGAAGTCTG | |
| | GTATAGTAGCTCTAACTATCGCGTT | | AGTGGTCTCGTGATTTTCGGCGGA | |
| | GACGAATACTATTACTACGGTATGG | | GGGACCAGGCTGACCGTCCTCGGT | |
| | ACGTCTGGGGCCAAGGGACCACGGT | | CAGCCCAAGGCTGCCCCCTCGGTC | |
| | CACCGTCTCCTCAGCATCCCCGACC | | ACTCTGTTCCCACCCTCCTCTGAGG | |
| | AGCCCCAAGGTCTTCCCGCTGAGCC | | AGCTTCAAGCCAACAAGGCCACAC | |
| | TCTGCAGCACCCAGCCAGATGGGAA | | TGGTGTGTCTCATAAGTGACTTCTA | |
| | CGTGGTCATCGCCTGCCTGGTCCAG | | CCCGGGAGCCGTGACAGTGGCCTG | |
| | GGCTTCTTCCCCCAGGAGCCACTCA | | GAAGGCAGATAGCAGCCCCGTCAA | |
| | GTGTGACCTGGAGCGAAAGCGGAC | | GGCGGGAGTGGAGACCACCACACC | |
| | AGGGCGTGACCGCCAGAAACTTCCC | | CTCCAAACAAAGCAACAACAAGTA | |
| | C | | CGCGGCCAGCAGCTAAGATCGGAA | |
| | | | GAGC | |
| S144-740 | CAGGTGCAGCTGGTGCAGTCTGGGG | 1679 | GAAGTTGTGTTGACGCAGTCTCCA | 1769 |
| | CTGAGGTGAAGAAGCCTGGGGCCTC | | GGCACCCTGTCTTTGTCTCCAGGGG | |
| | AGTGAAGGTCTCCTGCAAGGCTTCT | | AAAGAGCCACCCTCTCCTGCAGGG | |
| | GGATACACCTTCACCGGCTACTATA | | CCAGTCAGAGTGTTAGCAGCAGCT | |
| | TGCACTGGGTGCGACAGGCCCCTGG | | ACTTAGCCTGGTACCAGCAGAAAC | |
| | ACAAGGGCTTGAGTGGATGGGACG | | CTGGCCAGGCTCCCAGGCTCGTCA | |
| | GATCAACCCTAACAGTGGTGACACA | | TCTATGGTGCATCCAGCAGGGCCA | |
| | AACTATGCACAGAAGTTTCAGGGCA | | CTGGCATCCCAGACAGGTTCAGTG | |
| | GGGTCACCATGACCAGGGACACGTC | | GCAGTGGGTCTGGGACAGACTTCA | |
| | CATCAGCACAGCCTACATGGAGCTG | | CTCTCACCATCAGCAGACTGGAGC | |
| | AGCAGGCTGAGATCTGACGACACG | | CTGAAGATTTTGCAGTGTATTACTG | |
| | GCCGTGTATTACTGTGCGAGATTGG | | TCAGCAGTTTGGTAGCTCTCCCACC | |
| | GTAAAGGAATGGCAGCAGCCCGTA | | TTCGGCCGAGGGACACGACTGGAG | |
| | CTGTCTTTGACTCCTGGGGCCAGGG | | ATTAAACGAACTGTGGCTGCACCA | |
| | AACCCTGGTCACCGTCTCCTCAGCC | | TCTGTCTTCATCTTCCCGCCATCTG | |
| | TCCACCAAGGGCCCATCGGTCTTCC | | ATGAGCAGTTGAAATCTGGAACTG | |
| | CCCTGGCACCCTCCTCCAAGAGCAC | | CCTCTGTTGTGTGCCTGCTGAATAA | |
| | CTCTGGGGGCACAGCGGCCCTGGGC | | CTTCTATCCCAGAGAGGCCAAAGT | |
| | TGCCTGGTCAAGGACTACTTCCCCG | | ACAGTGGAAGGTGGATAACGCCCT | |
| | AACCGGTGACGGTGTCGTGGAACTC | | CCAATCGGGTAACTCCCAGGAGAG | |
| | AGGCGCCCTGACCAGCGGCGTGCAC | | TGTCACAGAGCAGGACAGCAAGGA | |
| | ACCTTCCCGGCTGTCCTACAGTCCTC | | CAGCACCTACAGCCTCAGCAGCAC | |
| | AGGA | | CCTGACGCTGAGCAAAGCAGACTA | |
| | | | CGAGAA | |
| S144-741 | CAGGTGCACCTGGTGCAGTCTGGGG | 1680 | CAGTCTGTGCTGACTCAGCCACCCT | 1770 |
| | CTGAGGTGAAGAAGCCTGGGGCCTC | | CAGCGTCTGGGACCCCCGGGCAGA | |
| | AGTGAAGGTCTCCTGCAAGGCTTCT | | GGGTCACCATCTCTTGTTCTGGAAG | |
| | GGATACACCTTCACCGGCTACTATA | | CAGCTCCAACATCGGAAGTAATAC | |
| | TGAACTGGGTGCGACAGGCCCCTGG | | TGTAAACTGGTACCAGCAGCTCCC | |
| | ACAAGGGCTTGAGTGGATGGGACG | | AGGAACGGCCCCCAAGCTCCTCAT | |
| | GATCAACCCTAACAGTGGTGGCACA | | CTATAGTAATAATCAGCGGCCCTC | |
| | AACTATGCACAGAAGTTTCAGGGCA | | AGGGGTCCCTGACCGATTCTCTGG | |
| | GGGTCACCATGACCAGGGACACGTC | | CTCCAAGTCTGGCACCTCAGCCTCC | |
| | CATCAGCACAGCCTACATGGAACTG | | CTGGCCATCAGTGGGCTCCAGTCT | |
| | AGCAGGCTGAGATCTGACGACGCG | | GAGGATGAGGCTGATTATTACTGT | |
| | GCCGTGTATTACTGTGCGAGAGCTG | | GCAGCATGGGATGACAGCCTGAAT | |
| | AGAGGTATAGCAGCAGCTGGTACA | | GGTGTGGTATTCGGCGGAGGGACC | |
| | ATCTTTACTACTGGGGCCAGGGAAC | | AAGCTGACCGTCCTAGGTCAGCCC | |
| | CCTGGTCACCGTCTCCTCAGCCTCC | | AAGGCTGCCCCCTCGGTCACTCTGT | |
| | ACCAAGGGCCCATCGGTCTTCCCCC | | TCCCGCCCTCCTCTGAGGAGCTTCA | |
| | TGGCACCCTCCTCCAAGAGCACCTC | | AGCCAACAAGGCCACACTGGTGTG | |
| | TGGGGGCACAGCGGCCCTGGGCTGC | | TCTCATAAGTGACTTCTACCCGGGA | |
| | CTGGTCAAGGACTACTTCCCCGAAC | | GCCGTGACAGTGGCCTGGAAGGCA | |
| | CGGTGACGGTGTCGTGGAACTCAGG | | GATAGCAGCCCCGTCAAGGCGGGA | |
| | CGCCCTGACCAGCGGCGTGCACACC | | GTGGAGACCACCACACCCTCCAAA | |
| | TTCCCGGCTGTCCTACAGTCCTCAG | | CAAAGCAACAACAAGTACGCGGCC | |
| | GA | | AGCAGCTATCTGAGCCTGACGCCT | |
| | | | GAGCAGTGGAAGTCCCACA | |
| S144-803 | GAGGTGCAGCTGGTGCAGTCTGGAG | 1681 | GACATCCAGATGACCCAGTCTCCTT | 1771 |
| | CAGAGGTGAAAAAGCCCGGGGAGT | | CCACCCTGTCTGCATCTGTAGGAG | |
| | CTCTGAAGATCTCCTGTAAGGGTTC | | ACAGAGTCACCATCACTTGCCGGG | |
| | TAGATACAGCTTTACCAGATACTGG | | CCAGTCAGAGTATTAGTAGTTGGTT | |
| | ATCGCCTGGGTGCGCCAGATGCCCG | | GGCCTGGTATCAGCAGAAACCAGG | |
| | GGAAAGGCCTGGAGTGGATGGGGA | | GAAAGCCCCTAAGCTCCTGATCTA | |
| | TCATCTATCCTGGTGACTCTGATACC | | TGATGCCTCCAGTTTGGAAAGTGG | |
| | AGATACAGCCCGTCCTTCCAAGGCC | | GGTCCCATCAAGGTTCAGCGGCAG | |
| | CGGTCACCATCTCAGCCGACAAGTC | | TGGATCTGGGACAGAATTCACTCT | |
| | CATCAGCACCGCCTACCTGCAGTGG | | CACCATCAGCAGCCTGCAGCCTGA | |
| | AGCAGCCTGAAGGCCTCGGACACCG | | TGATTTTGCAACTTATTACTGCCAA | |
| | CCATATATTACTGTGCGAGACTCCC | | CAGTATAATATTTACCCGTACACTT | |
| | GAACAGTAACTACGTTGACTACTGG | | TTGGCCAGGGGACCAAGCTGGACA | |
| | GGCCAGGGAACCCTGGTCACCGTCT | | TCAAACGAACTGTGGCTGCACCAT | |
| | CCTCAGCCTCCACCAAGGGCCCATC | | CTGTCTTCATCTTCCCGCCATCTGA | |
| | GGTCTTCCCCCTGGCACCCTCCTCCA | | TGAGCAGTTGAAATCTGGAACTGC | |
| | AGAGCACCTCTGGGGGCACAGCGG | | CTCTGTTGTGTGCCTGCTGAATAAC | |
| | CCCTGGGCTGCCTGGTCAAGGACTA | | TTCTATCCCAGAGAGGCCAAAGTA | |
| | CTTCCCCGAACCGGTGACGGTGTCG | | CAGTGGAAGGTGGATAACGCCCTC | |
| | TGGAACTCAGGCGCCCTGACCAGCG | | CAATCGGGTAACTCCCAGGAGAGT | |
| | GCGTGCACACCTTCCCGGCTGTCCT | | GTCACAGAGCAGGACAGCAAGGAC | |
| | ACAGTCCTCAGGACTCTACTCCCTC | | AGCACCTACAGCCTCAGCAGCACC | |
| | AGCAGCGTGGTGACCGTGCCCTCCA | | CTGACGCTGAGCAAAGCAGACTAC | |
| | GCAGCTTGGGCACCCAGACCTACAT | | GAGAA | |
| | CTGCAACGTGAATCACAAGCCCAGC | | | |
| | AACACCAAGGTGGACAA | | | |
| S144-843 | CAGGTGCAGCTGGTGGAGTCTGGGG | 1682 | GAAATTGTGTTGACGCAGTCTCCA | 1772 |
| | GAGGCGTGGTCCAGCCTGGGGGGTC | | GGCACCCTGTCTTTGTCTCCAGGGG | |
| | CGTAAGACTCTCCTGTGCAGCGTCT | | AAAGAGCCACCCTCTCCTGCAGGG | |
| | GGATTCGACTTCACTAATAATGGCA | | CCAGTCAGACTGTTACCAGCAGGT | |
| | TGTATTGGGTCCGCCAGGCTCCAGG | | ACTTAGCCTGGTATCAGCAGAAGC | |
| | CAAGGGGCTGGAGTGGGTGGCATTT | | CTGGCCAGGCTCCCAGGCTCCTCAT | |
| | ATACGGTATGATGGAAATAAACAA | | CTATGGTGCATCCACCAGGGCCAC | |
| | GACTATGCAGACTCCGTGAAGGGCC | | TGGCATCCCAGACAGGTTCAGTGG | |
| | GATTCACCATCTCCAGAGACAATTC | | CAGTGGGTCTGGGACAGACTTCAC | |
| | CAAAAACACTCTGTATCTGCAAATG | | TCTCACCATCAGCAGACTGGAGCC | |
| | AGCAGCCTTAGACCTGAGGACACGG | | TGAAGATTTTGCAGTGTATTACTGT | |
| | CTGTATATTACTGTGCGAAAGGTGT | | CAGCAGTATGGTAATTCACCTCCGT | |
| | TTATACTGAAAATTACGGCTGGGGC | | ACACTTTTGGCCAGGGGACCAAGC | |
| | CAGGGAACCCTGGTCACCGTCTCCT | | TGGAGATCAAACGAACTGTGGCTG | |
| | CAGGGACCACGGTCACCGTCTCCTC | | CACCATCTGTCTTCATCTTCCCGCC | |
| | AGCCTCCACCAAGGGCCCATCGGTC | | ATCTGATGAGCAGTTGAAATCTGG | |
| | TTCCCCCTGGCGCCCTGCTCCAGGA | | AACTGCCTCTGTTGTGTGCCTGCTG | |
| | GCACCTCCGAGAGCACAGCGGCCCT | | AATAACTTCTATCCCAGAGAGGCC | |
| | GGGCTGCCTGGTCAAGGACTACTTC | | AAAGTACAGTGGAAGGTGGATAAC | |
| | CCCGAACCGGTGACGGTGTCGTGGA | | GCCCTCCAATCGGGTAACTCCCAG | |
| | ACTCAGGCGCTCTGACCAGCGGCGT | | GAGAGTGTCACAGAGCAGGACAGC | |
| | GCACACCTTCCCAGCTGTCCTACAG | | AAGGACAGCACCTACAGCCTCAGC | |
| | TCCTCAGGACTCTACTCCCTCAGCA | | AGCACCCTGACGCTGAGCAAAGCA | |
| | GCGTGGTGACCGTGCCCTCCAGCAA | | GACTACGAGAA | |
| | CTTCGGCACCCAGACCTACACCTGC | | | |
| | AACGTAGATCACAAGCCCAGCAAC | | | |
| | ACCAAGGTGGACAA | | | |
| S144-877 | CAGGTGCAGCTGGTGGAGTCTGGGG | 1683 | GACATCCAGATGACCCAGTCTCCA | 1773 |
| | GAGGCGTGGTCCAGCCTGGGAGGTC | | TCCTCCCTGTCTGCATCTGTAGGAG | |
| | CCTGAGACTCTCCTGTGCAGCCTCT | | ACAGAGTCACCATCACTTGCCAGG | |
| | GGATTCACCTTCAGTACCTATGGCA | | CGAGTCAGGACATTAGCAACTATT | |
| | TGCACTGGGTCCGCCAGGCTCCAGG | | TAAATTGGTATCAGCAGAAACCAG | |
| | CAAGGGGCTGGAGTGGGTGGCAGTT | | GGAAAGCCCCTAAGCTCCTGATCT | |
| | ATATCATATGATGGAAGTAATAAAT | | ACGATGCATCGAATTTGGAAACAG | |
| | ATTATGCAGACTCCGTGAAGGGCCG | | GGGTCCCATCAAGGTTCAGTGGAA | |
| | ATTCACCATCTCCAGAGACAATTCC | | GTGGATCTGGGACAGATTTTAGTTT | |
| | AAGAACACGCTGTATCTGCAAATGA | | TAGTATCAGCAGCCTGCAGCCTGA | |
| | ACAGCCTGAGAGCTGAGGACACGG | | AGATATTGCAACATATTACTGTCA | |
| | CTGTGTATTACTGTGCGAAACAGCA | | ACAGTATGATAATGTCCCTCTTACT | |
| | AGGCACCTATTGCAGTGGTGGTAAC | | TTCGGCGGAGGGACCAAGGTGGAG | |
| | TGCTACTCGGGATATTTTGACTACT | | ATCAAACGAACTGTGGCTGCACCA | |
| | GGGGCCAGGGAACCCTGGTCACCGT | | TCTGTCTTCATCTTCCCGCCATCTG | |
| | CTCCTCAGCCTCCACCAAGGGCCCA | | ATGAGCAGTTGAAATCTGGAACTG | |
| | TCGGTCTTCCCCCTGGCACCCTCCTC | | CCTCTGTTGTGTGCCTGCTGAATAA | |
| | CAAGAGCACCTCTGGGGGCACAGC | | CTTCTATCCCAGAGAGGCCAAAGT | |
| | GGCCCTGGGCTGCCTGGTCAAGGAC | | ACAGTGGAAGGTGGATAACGCCCT | |
| | TACTTCCCCGAACCGGTGACGGTGT | | CCAATCGGGTAACTCCCAGGAGAG | |
| | CGTGGAACTCAGGCGCCCTGACCAG | | TGTCACAGAGCAGGACAGCAAGGA | |
| | CGGCGTGCACACCTTCCCGGCTGTC | | CAGCACCTACAGCCTCAGCAGCAC | |
| | CTACAGTCCTCAGGACTCTACTCCC | | CCTGACGCTGAGCAAAGCAGACTA | |
| | TCAGCAGCGTGGTGACCGTGCCCTC | | CGAGAA | |
| | CAGCAGCTTGGGCACCCAGACCTAC | | | |
| | ATCTGCA | | | |
| S144-952 | CAGGTTCAGCTGGTGCAGTCTGGAG | 1684 | GACATCGTGATGACCCAGTCTCCA | 1774 |
| | CTGAGGTGAAGAAGCCTGGGGCCTC | | GACTCCCTGGCTGTGTCTCTGGGCG | |
| | AGTGAAGGTCTCCTGCACGGCTTCT | | AGAGGGCCACCATCAACTGCAAGT | |
| | GGTTACACCGTTACCAGTTATGGTA | | CCAGCCAGAGTGTTTTAAACAGCT | |
| | TCAGCTGGGTGCGACAGGCCCCTGG | | CCAACAATAAGAACTACTTAGCTT | |
| | ACAAGGGCTTGAGTGGATGGGATG | | GGTACCAGCAGAAACCAGGACAGC | |
| | GATCAGCACTTACAATGGTAACACA | | CTCCTAAGCTGCTCATTTACTGGGC | |
| | AACTATGCACAGAAGCTCCAGGGCA | | ATCTACCCGGGAATCCGGGGTCCC | |
| | GAGTCACCATGACCACAGACACATC | | TGACCGATTCAGTGGCAGCGGGTC | |
| | CACGAGCACAGCCTACATGGAGCTG | | TGGGACAGATTTCACTCTCACCATC | |
| | AGGAGCCTGAGATCTGACGACACG | | AGCAGCCTGCAGGCTGAAGATGTG | |
| | GCCGTGTATTACTGTGCGAGAGAAT | | GCAGTTTATTACTGTCAGCAGTATT | |
| | ACAGCTATGGTTACCGACTGGCCTA | | ATAGTACTCCTCAGACGTTCGGCC | |
| | CTTTGACTACTGGGGCCAGGGAACC | | AAGGGACCAAGGTGGAAATCAAAC | |
| | CTGGTCACCGTCTCCTCAGGGAGTG | | GAACTGTGGCTGCACCATCTGTCTT | |
| | CATCCGCCCCAACCCTTTTCCCCCTC | | CATCTTCCCGCCATCTGATGAGCAG | |
| | GTCTCCTGTGAGAATTCCCCGTCGG | | TTGAAATCTGGAACTGCCTCTGTTG | |
| | ATACGAGCAGCGTGGCCGTTGGCTG | | TGTGCCTGCTGAATAACTTCTATCC | |
| | CCTCGCACAGGACTTCCTTCCCGAC | | CAGAGAGGCCAAAGTACAGTGGAA | |
| | TCCATCACTTTCTCCTGGAAATACA | | GGTGGATAACGCCCTCCAATCGGG | |
| | AGAACAACTCTGACATCAGCAGCAC | | TAACTCCCAGGAGAGTGTCACAGA | |
| | CCGGGGCTTCCCATCAGTCCTGAGA | | GCAGGACAGCAAGGACAGCACCTA | |
| | GGGGGCAAGTACGCAGCCACCTCAC | | CAGCCTCAGCAGCACCCTGACGCT | |
| | AGGTGCTGCTGCCTTCCAAGGACGT | | GAGCAAAGCAGACTACGAGA | |
| | CATG | | | |
| S144-971 | GAGGTGCAGCTGGTGGAGTCTGGGG | 1685 | GACATCGTGATGACCCAGTCTCCA | 1775 |
| | GAGGCTTGGTCCAGCCTGGGGGGTC | | GACTCCCTGGCTGTGTCTCTGGGCG | |
| | CCTGAGAATCTCTTGTTCAGCCTCTG | | AGAGGGCCACCATCAACTGCAAGT | |
| | GATTCACCTTCAGTAGATATGCTAT | | CCAGCCAGAGTGTTTTATACAGCTC | |
| | GCACTGGGTCCGCCAGGCTCCAGGG | | CAACAATAAGAACTTCTTAACTTG | |
| | AAGGGACTGGAATATGTTTCAGCTA | | GTACCAGCAGAAACCAGGACAGCC | |
| | TTAGGAGTAATGGGGGTAGCACATA | | TCCTAAGCTGCTCATTTACTGGGCA | |
| | CTACGCAGACTCCGTGAGGGGCAGA | | TCTACCCGGGAATCCGGGGTCCCT | |
| | TTCACCATCTCCAGAGACAATTCCA | | GACCGATTCAGTGGCAGCGGGTCT | |
| | GGAACACGCTGTATCTTCAAATGAG | | GGGACAGATTTCACTCTCACCATC | |
| | CAGTCTGAGAGCTGAGGACACGGCT | | AGCAGCCTGCAGGCTGAAGATGTG | |
| | GTGTATTACTGTGTGATAATAAACA | | GCAGTTTATTACTGTCAGCAATATT | |
| | ATTTAGCAGCAGCTGGTACCCGTTT | | ATACTACTCCGTGGACGTTCGGCC | |
| | TGACTACTGGGGCCAGGGAACCCTG | | AAGGGACCAAGGTGGAAATCAAAC | |
| | GTCACCGTCTCCTCAGCCTCCACCA | | GAACTGTGGCTGCACCATCTGTCTT | |
| | AGGGCCCATCGGTCTTCCCCCTGGC | | CATCTTCCCGCCATCTGATGAGCAG | |
| | ACCCTCCTCCAAGAGCACCTCTGGG | | TTGAAATCTGGAACTGCCTCTGTTG | |
| | GGCACAGCGGCCCTGGGCTGCCTGG | | TGTGCCTGCTGAATAACTTCTATCC | |
| | TCAAGGACTACTTCCCCGAACCGGT | | CAGAGAGGCCAAAGTACAGTGGAA | |
| | GACGGTGTCGTGGAACTCAGGCGCC | | GGTGGATAACGCCCTCCAATCGGG | |
| | CTGACCAGCGGCGTGCACACCTTCC | | TAACTCCCAGGAGAGTGTCACAGA | |
| | CGGCTGTCCTACAGTCCTCAGGA | | GCAGGACAGCAAGGACAGCACCTA | |
| | | | CAGCCTCAGCAGCACCCTGACGCT | |
| | | | GAGCAAAGCAGACTACGAGAA | |
| S144-1036 | CAGGTGCAGCTACAGCAGTGGGGC | 1686 | GACATCGTGATGACCCAGTCTCCA | 1776 |
| | GCAGGGCTGTTGAAGCCTTCGGAGA | | GACTCCCTGGCTGTGTCTCTGGGCG | |
| | CCCTGTCCCTCACCTGCGCTGTCTAT | | AGAGGGCCACCATCAACTGCAACT | |
| | GGTGGGTCCTTCAGTGGTTACTTCT | | CCAGCCAGAGTGTTTTATACAGCTC | |
| | GGAGCTGGATCCGCCAGCCCCCAGG | | CATCAATAAGAACTACTTAGCTTG | |
| | GAAGGGGCTGGAGTGGATTGGGGA | | GTACCAGCAGAAACCAGCACAGCC | |
| | AATCAATCATAGTGGAAGCACCAAC | | TCCTAAGGTGCTCATTTACTGGGCA | |
| | TACAACCCGTCCCTCAAGAGTCGAG | | TCTACCCGGGAATCCGGGGTCCCT | |
| | TCACCATATCAGTAGACACGTCCAA | | GACCGATTCAGTGGCAGCGGGTCT | |
| | GAACCAGTTCTCCCTGAAGCTGAGC | | GGGACAGATTTCACTCTCACCATC | |
| | TCTGTGACCGCCGCGGACACGGCTG | | AGCAGCCTGCAGGCTGAAGATGTG | |
| | TGTATTACTGTGCGAGAGCGCCCTA | | GCAGTTTATTACTGTCAGCAATATT | |
| | TTACGATTTCTTGCGGGAAGGAAAC | | ATAGGACTCCCTGGACGTTCGGCC | |
| | TGGTTCGACCCCTGGGGCCAGGGAA | | AAGGGACCAAGGTGGAAATCAAAC | |
| | CCCTGGTCACCGTCTCCTCAGCCTCC | | GAACTGTGGCTGCACCATCTGTCTT | |
| | ACCAAGGGCCCATCGGTCTTCCCCC | | CATCTTCCCGCCATCTGATGAGCAG | |
| | TGGCACCCTCCTCCAAGAGCACCTC | | TTGAAATCTGGAACTGCCTCTGTTG | |
| | TGGGGGCACAGCGGCCCTGGGCTGC | | TGTGCCTGCTGAATAACTTCTATCC | |
| | CTGGTCAAGGACTACTTCCCCGAAC | | CAGAGAGGCCAAAGTACAGTGGAA | |
| | CGGTGACGGTGTCGTGGAACTCAGG | | GGTGGATAACGCCCTCCAATCGGG | |
| | CGCCCTGACCAGCGGCGTGCACACC | | TAACTCCCAGGAGAGTGTCACAGA | |
| | TTCCCGGCTGTCCTACAGTCCTCAG | | GCAGGACAGCAAGGACAGCACCTA | |
| | GACTCTACTCCCTCAGCAGCGTGGT | | CAGCCTCAGCAGCACCCTGACGCT | |
| | GACCGTGCCCTCCAGCAGCTTGGGC | | GAGCAAAGCAGACTACGAGAA | |
| | ACCCAGACCTACATCTGCAACGTGA | | | |
| | ATCACAAGCCCAGC | | | |
| S144-1079 | CAGGTCCAGCTGGTGCAATCTGGGG | 1687 | GAAATTGTGTTGACGCAGTCTCCA | 1777 |
| | CTGAGGTGAAGAAGCCTGGGTCCTC | | GGCACCCTGTCTTTGTCTCCAGGGG | |
| | GGTGAAGGTCTCCTGCAAGGCTTCT | | AAAGAGCCACCCTCTCCTGCAGGG | |
| | GGAGACACCTTCGGCAGCTATAGTA | | CCAGTCAGAGTGTTAGCAGCAACT | |
| | TCACCTGGGTGCGACAGGCCCCTGG | | ACTTAGCCTGGTACCAGCAGAAAC | |
| | ACAAGGACTTGAGTGGATGGGAAG | | CTGGCCAGGCTCCCAGGCTCCTCAT | |
| | GATCATCCCTGTCCTTGGTATAGCA | | CTATGGTGCATCCAGCAGGGCCAC | |
| | AACTACGCACAGAAGTTCCAGGGCA | | TGGCATCCCAGAGAGGTTCAGTGG | |
| | GAGTCACGATTACCGCGGACAAATC | | CAGTGGGTCTGGGACAGACTTCAC | |
| | CACGAGCACAGCCTACATGGAGCTG | | TCTCACCATCAGCAGACTGGAGCC | |
| | AGCAGCCTGAGATCTGAGGACACG | | TGAAGATTTTGCAGTGTATTACTGT | |
| | GCCGTGTATTACTGTGCGGGAGGGG | | CAGCAGTATGGTAGGTCACCGTAC | |
| | GTTGTAGTGGTGGTAACTGCTACTC | | ACTTTTGGCCAGGGGACCAAGCTG | |
| | GTGGTACAACTGGTTCGACCCCTGG | | GAGATCAAACGAACTGTGGCTGCA | |
| | GGCCAGGGATCCCTGGTCACCGTCT | | CCATCTGTCTTCATCTTCCCGCCAT | |
| | CCTCAGCCTCCACCAAGGGCCCATC | | CTGATGAGCAGTTGAAATCTGGAA | |
| | GGTCTTCCCCCTGGCACCCTCCTCCA | | CTGCCTCTGTTGTGTGCCTGCTGAA | |
| | AGAGCACCTCTGGGGGCACAGCGG | | TAACTTCTATCCCAGAGAGGCCAA | |
| | CCCTGGGCTGCCTGGTCAAGGACTA | | AGTACAGTGGAAGGTGGATAACGC | |
| | CTTCCCCGAACCGGTGACGGTGTCG | | CCTCCAATCGGGTAACTCCCAGGA | |
| | TGGAACTCAGGCGCCCTGACCAGCG | | GAGTGTCACAGAGCAGGACAGCAA | |
| | GCGTGCACACCTTCCCGGCTGTCCT | | GGACAGCACTTACAGCCTCAGCAG | |
| | ACAGTCCTCAGGA | | CACCCTGACGCTGAGCAAAGCAGA | |
| | | | CTACGAGAA | |
| S144-1299 | CAGGTGCAGCTGCAGGAGTCGGGCC | 1688 | CAGTCTGTGCTGACTCAGCCACCCT | 1778 |
| | CAGGACTGGTGAAGCCTTCGGAGAC | | CAGCGTCTGGGACCCCCGGGCAGA | |
| | CCTGTCCCTCACCTGCACTGTCTCTG | | GGGTCACCATCTCTTGTTCTGGAAG | |
| | GTGGCTCCATCAGTAGTTACTACTG | | CAGCTCCAACATCGGAAGTAATTA | |
| | GAGCTGGATCCGGCAGCCCCCAGGG | | TGTATACTGGTACCAGCAGCTCCC | |
| | AAGGGACTGGAGTGGATTGGGTATA | | AGGAACGGCCCCCAAACTCCTCAT | |
| | TCAATTACAGGGGGATCACCAACTA | | CTATAGGAATAATCAGCGGCCCTC | |
| | CAACCCCTCCCTCAAGAGTCGAGTC | | AGGGGTCCCTGACCGATTCTCTGG | |
| | ACCATATCAGTAGACATGTCCAAGA | | CTCCAAGTCTGGCACCTCAGCCTCC | |
| | ACCAGTTCTCCCTGAAGCTGAGCTC | | CTGGCCATCAGTGGGCTCCGGTCC | |
| | TGTGACCGCCGCAGACACGGCCGTG | | GAGGATGAGGCTGATTATTACTGT | |
| | TATTCCTGTGCGAGACTAGCAGTGG | | GCAGCATGGGATGACAGCCTGAGT | |
| | CTAGTCGAGGGACCGTTGACTACTG | | GTTAATGTGGTATTCGGCGGAGGG | |
| | GGGCCAGGGAACCCTGGTCACCGTC | | ACCAAGCTGACCGTCCTAGGTCAG | |
| | TCCTCAGCCTCCACCAAGGGCCCAT | | CCCAAGGCTGCCCCCTCGGTCACTC | |
| | CGGTCTTCCCCCTGGCACCCTCCTCC | | TGTTCCCGCCCTCCTCTGAGGAGCT | |
| | AAGAGCACCTCTGGGGGCACAGCG | | TCAAGCCAACAAGGCCACACTGGT | |
| | GCCCTGGGCTGCCTGGTCAAGGACT | | GTGTCTCATAAGTGACTTCTACCCG | |
| | ACTTCCCCGAACCGGTGACGGTGTC | | GGAGCCGTGACAGTGGCCTGGAAG | |
| | GTGGAACTCAGGCGCTCTGACCAGC | | GCAGATAGCAGCCCCGTCAAGGCG | |
| | GGCGTGCACACCTTCCCAGCTGTCC | | GGAGTGGAGACCACCAAACCCTCC | |
| | TACAGTCCTCAGGACTCTACTCCCT | | AAACAGAGCAACAACAAGTACGCG | |
| | CAGCAGCGTGGTGACCGTGCCCTCC | | GCCAGCAGCTACCTGAGCCTGACG | |
| | AGCAACTTCGGCACCCAGACCTACA | | CCTGAGCAGTGGAAGTCCCACA | |
| | CCTGCAACGTAGATCACAAGCCCAG | | | |
| | CAACACCAAGGTGGAC | | | |
| S144-1339 | CAGGTGCAGCTGGTGCAGTCTGGGA | 1689 | CAGTCTGCCCTGACTCAGCCTGCCT | 1779 |
| | CTGAGGTGAAGAAGCCTGGGGCCTC | | CCGTGTCTGGGTCTCCTGGACAGTC | |
| | AGTGAAGGTCTCCTGCAAGGCTTCT | | GATCACCATCTCCTGCACTGGAAC | |
| | GGATACACCTTCACCGACTACTATA | | CAACAGTGACGTTGGTGGTTATAA | |
| | TGCACTGGGTGCGACAGGCCCCTGG | | CTATGTCTCCTGGTACCAACAACAC | |
| | ACAAGGGCTTGAGTGGATGGGACG | | CCAGGCAAAGCCCCCAGACTCATG | |
| | GATCAACCCTACCAGTGGTGGCACA | | ATTTATGATGTCAGTAATCGGCCCT | |
| | AACTATCCACAGAAGTTTCAGGGCA | | CAGGGGTTTCTAATCGCTTCTCTGG | |
| | GTGTCACCATGACCAGGGACACGTC | | CTCCAAGTCTGGCAACACGGCCTC | |
| | CCTCAGCACAGTCTACATGGAACTG | | CCTGACCATCTCTGGGCTCCAGGCT | |
| | AGCGGGCTGAGATCTGACGACACG | | GAGGACGAGGCTGATTATTACTGC | |
| | GCCGTCTATTATTGTGCGAGAGAGA | | AGCTCATATACAAGCAGCAGCACT | |
| | GGGTTACTCTGATTCAGGGAAAGAA | | CTCGTGGTTTTCGGCGGAGGGACC | |
| | CCACTACTACATGGACGTCTGGGGC | | AAGCTGACCGTCCTAGGTCAGCCC | |
| | ACAGGGACCACGGTCACCGTCTCCT | | AAGGCTGCCCCCTCGGTCACTCTGT | |
| | CAGCCTCCACCAAGGGCCCATCGGT | | TCCCGCCCTCCTCTGAGGAGCTTCA | |
| | CTTCCCCCTGGCACCCTCCTCCAAG | | AGCCAACAAGGCCACACTGGTGTG | |
| | AGCACCTCTGGGGGCACAGCGGCCC | | TCTCATAAGTGACTTCTACCCGGGA | |
| | TGGGCTGCCTGGTCAAGGACTACTT | | GCCGTGACAGTGGCCTGGAAGGCA | |
| | CCCCGAACCGGTGACGGTGTCGTGG | | GATAGCAGCCCCGTCAAGGCGGGA | |
| | AACTCAGGCGCCCTGACCAGCGGCG | | GTGGAGACCACCACACCCTCCAAA | |
| | TGCACACCTTCCCGGCTGTCCTACA | | CAAAGCAACAACAAGTACGCGGCC | |
| | GTCCTCAGGA | | AGCAGCTATCTGAGCCTGACGCCT | |
| | | | GAGCAGTGGAAGTCCCACA | |
| S144-1406 | CAGGTCCAGCTTGTGCAGTCTGGGG | 1690 | GACATCCAGATGACCCAGTCTCCTT | 1780 |
| | CTGAGGTGAAGAAGCCTGGGGCCTC | | CCACCCTGTCTGCATCTGTAGGAG | |
| | AGTGAAGGTTTCCTGCAAGGCTTCT | | ACAGAGTCACCATCACTTGCCGGG | |
| | GGATATACCTTCACTACCTATGCTA | | CCAGTCAGAGTATTAGTAGCTGGT | |
| | TGCATTGGGTGCGCCAGGCCCCCGG | | TGGCCTGGTATCAGCAGAAACCAG | |
| | ACAAAGGCTTGAGTGGATGGGATG | | GGAAAGCCCCTAAGCTCCTGATCT | |
| | GATCAACGCTGGCAATGGTAACACA | | ATGATGCCTCCAGTTTGGAAAGTG | |
| | AAATATTCACAGAACTTCCAGGGCA | | GGGTCCCATCAAGGTTCAGCGGCA | |
| | GAGTCACCATTACCAGGGACACATC | | GTGGATCTGGGACAGAATTCACTC | |
| | CGCGAGCACAGCCTACATGGAGCTG | | TCACCATCAGCAGCCTGCAGCCTG | |
| | AGCAGCCTGAGATCTGAAGACACG | | ATGATTTTGCAACTTATTACTGCCA | |
| | GCTGTGTATTACTGTGCGAGTCTCG | | ACAGTATAATAGTTATCCGTGGAC | |
| | TGGGTGGGGATAGCAGCAGCTGGTA | | GTTCGGCCAAGGGACCAAGGTGGA | |
| | TGACTACATGGACGTCTGGGGCAAA | | AATCAAACGAACTGTGGCTGCACC | |
| | GGGACCACGGTCACCGTCTCCTCAG | | ATCTGTCTTCATCTTCCCGCCATCT | |
| | CCTCCACCAAGGGCCCATCGGTCTT | | GATGAGCAGTTGAAATCTGGAACT | |
| | CCCCCTGGCGCCCTGCTCCAGGAGC | | GCCTCTGTTGTGTGCCTGCTGAATA | |
| | ACCTCCGAGAGCACAGCGGCCCTGG | | ACTTCTATCCCAGAGAGGCCAAAG | |
| | GCTGCCTGGTCAAGGACTACTTCCC | | TACAGTGGAAGGTGGATAACGCCC | |
| | CGAACCGGTGACGGTGTCGTGGAAC | | TCCAATCGGGTAACTCCCAGGAGA | |
| | TCAGGCGCTCTGACCAGCGGCGTGC | | GTGTCACAGAGCAGGACAGCAAGG | |
| | ACACCTTCCCAGCTGTCCTACAGTC | | ACAGCACCTACAGCCTCAGCAGCA | |
| | CTCAGGACTCTACTCCCTCAGCAGC | | CCCTGACGCTGAGCAAAGCAGACT | |
| | GTGGTGACCGTGCCCTCCAGCAACT | | ACGAGAA | |
| | TCGG | | | |
| S144-1407 | CAGGTCCAGCTGGTGCAATCTGGGG | 1691 | GACATCCAGATGACCCAGTCTCCTT | 1781 |
| | CTGAGGTGAAGAAGCCTGGGTCCTC | | CCACCCTGTCTGCATCTGTAGGAG | |
| | GGTGAAGGTCTCCTGCAAGGCTTCT | | ACAGAGTCACCATCACTTGCCGGG | |
| | GGAGGCACCTTCAGCAGCTATACTA | | CCAGTCAGAGTATTAGTAGCTGGT | |
| | TCAGCTGGGTGCGACAGGCCCCTGG | | TGGCCTGGTATCAGCAGAAACCAG | |
| | ACAAGGCCTTGAGTGGATGGGAAG | | GGAAAGCCCCTAAGCTCCTGATCT | |
| | GATCATCCCTGTCCGTGATATAGCA | | ATGATGCCTCCAGTTTGGAAAGTG | |
| | AACTACGCACAGAAGTTCCAGGGCA | | GGGTCCCATCAAGGTTCAGCGGCA | |
| | GAGTCACGATTACCGCGGACAAATC | | GTGGATCTGGGACAGAATTCACTC | |
| | CACGAGGACAGCCTACATGGAGGT | | TCACCGTCAGCAGCCTGCAGCCTG | |
| | GAGCAGCCTGAGATCTGAGGACAC | | ATGATTTTGCAACTTATTACTGCCA | |
| | GGCCGTGTATTACTGTGCGGCAACG | | ACAGTATAATAATTATTCTCCCATC | |
| | GAGCTCCGCTCGGATGGTCTTGACA | | ACTTTTGGCCAGGGGACCAAGCTG | |
| | TCTGGGGCCAAGGGACAATGGTCAC | | GAGATCAAACGAACTGTGGCTGCA | |
| | CGTCTCTTCAGCCTCCACCAAGGGC | | CCATCTGTCTTCATCTTCCCGCCAT | |
| | CCATCGGTCTTCCCCCTGGCACCCTC | | CTGATGAGCAGTTGAAATCTGGAA | |
| | CTCCAAGAGCACCTCTGGGGGCACA | | CTGCCTCTGTTGTGTGCCTGCTGAA | |
| | GCGGCCCTGGGCTGCCTGGTCAAGG | | TAACTTCTATCCCAGAGAGGCCAA | |
| | ACTACTTCCCCGAACCGGTGACGGT | | AGTACAGTGGAAGGTGGATAACGC | |
| | GTCGTGGAACTCAGGCGCCCTGACC | | CCTCCAATCGGGTAACTCCCAGGA | |
| | AGCGGCGTGCACACCTTCCCGGCTG | | GAGTGTCACAGAGCAGGACAGCAA | |
| | TCCTACAGTCCTCAGGA | | GGACAGCACCTACAGCCTCAGCAG | |
| | | | CACCCTGACGCTGAGCAAAGCAGA | |
| | | | CTACGAGAA | |
| S144-1569 | CAGGTTCAGCTGGTGCAGTCTGGAG | 1692 | CAGCCTGTGCTGACTCAGCCACCTT | 1782 |
| | CTGAGGTGAAGAAGCCTGGGGCCTC | | CTGCATCAGCCTCCCTGGGAGCCTC | |
| | AGTGAAGGTCTCCTGCAAGGCTTCT | | GGTCACACTCACCTGCACCCTGAG | |
| | GGTTACACCTTTTCCAACTACGGTA | | CAGCGGCTACAGTAATTATAAAGT | |
| | TCAGCTGGGTGCGACAGGCCCCTGG | | GGACTGGTACCAGCAGAGACCAGG | |
| | ACAAGGGCTTGAGTGGATGGGATG | | GAAGGGCCCCCAGTTTGTGATGCG | |
| | GATCAGCGCTTACAATGGTAACACT | | AGTGGGCACTGGTGGGATTGTGGG | |
| | AAGTATCCACAAAAGCTCCAGGGCA | | ATCCAAGGGGGATGGCATCCCTGA | |
| | GAGTCACCATGAGCACAGACACATC | | TCGCTTCTCAGTCTTGGGCTCAGGC | |
| | CACGAGCACAGCCTACATGGAGCTG | | CTGAATCGGTACCTGACCATCAAG | |
| | AGGAGCCTGAGATCTGACGACACG | | AACATCCAGGAAGAGGATGAGAGT | |
| | GCCGTGTATTACTGTGCGAGAGAGA | | GACTACCACTGTGGGGCAGACCAT | |
| | CGCGGTACGGTATGGACGTCTGGGG | | GGCAGTGGGAGCAACTTCGTTCGG | |
| | CCAAGGGACCACGGTCACCGTCTCC | | GTGTTCGGCGGAGGGACCAAGCTG | |
| | TCAGCCTCCACCAAGGGCCCATCGG | | ACCGTCCTAGGTCAGCCCAAGGCT | |
| | TCTTCCCCCTGGCACCCTCCTCCAAG | | GCCCCCTCGGTCACTCTGTTCCCAC | |
| | AGCACCTCTGGGGGCACAGCGGCCC | | CCTCCTCTGAGGAGCTTCAAGCCA | |
| | TGGGCTGCCTGGTCAAGGACTACTT | | ACAAGGCCACACTGGTGTGTCTCA | |
| | CCCCGAACCGGTGACGGTGTCGTGG | | TAAGTGACTTCTACCCGGGAGCCG | |
| | AACTCAGGCGCCCTGACCAGCGGCG | | TGACAGTGGCCTGGAAGGCAGATA | |
| | TGCACACCTTCCCGGCTGTCCTACA | | GCAGCCCCGTCAAGGCGGGAGTGG | |
| | GTCCTCAGGA | | AGACCACCACACCCTCCAAACAAA | |
| | | | GCAACAACAAGTACGCGGCCAGCA | |
| | | | GCTACCTGAGCCTGACGCCTGAGC | |
| | | | AGTGGAAGTCCCAC | |
| S144-1641 | GAGGTGCAGCTGGTGCAGTCTGGAG | 1693 | GACATCCAGATGACCCAGTCTCCTT | 1783 |
| | CAGAGGTGAAAAAGCCCGGGGAGT | | CCACCCTGTCTGCATCTGTAGGAG | |
| | CTCTGAAGATCTCCTGTAAGGGTTC | | AGAGAGTCACCATCACTTGCCGGG | |
| | TGGATACACCTTTACCAGCTACTGG | | CCAGTCAGAGTATTAGTAGGTGGT | |
| | ATCGGCTGGGTGCGCCAGATGCCCG | | TGGCCTGGTATCAGCAGAAACCAG | |
| | GGAAAGGCCTGGAGTGGATGGGGA | | GGAAAGCCCCTAAACTCCTTATCT | |
| | TCATCTATCTTGGTGACTCTGATACG | | ATGATGCCTCCAGTTTGGAAAGTG | |
| | AGATACAGCCCGTCCTTCCAAGGCC | | GGGTCCCATCAAGGTTCAGCGGCA | |
| | AGGTCACCATCTCAGCCGACAAGTC | | GTGGATCTGGGACAGAATTCACTC | |
| | CATCAGCACCGCCTACCTGCAGTGG | | TCACCATCAGCAGCCTGCAGCCTG | |
| | AACAGCCTGAAGGCCTCGGACACCG | | ATGATTTTGCAACTTATCACTGCCA | |
| | CCATGTATTACTGTGCGAGACAGGT | | CCAGTATAGTACTTATTCGCTCACT | |
| | TACCGGAACTACGAGCTGGTTCGAC | | TTCGGCGGAGGGACCAAGGTGGAC | |
| | CCCTGGGGCCAGGGAACCCTGGTCA | | ATCAAACGAACTGTGGCTGCACCA | |
| | CCGTCTCCTCAGCCTCCACCAAGGG | | TCTGTCTTCATCTTCCCGCCATCTG | |
| | CCCATCGGTCTTCCCCCTGGCACCCT | | ATGAGCAGTTGAAATCTGGAACTG | |
| | CCTCCAAGAGCACCTCTGGGGGCAC | | CCTCTGTTGTGTGCCTGCTGAATAA | |
| | AGCGGCCCTGGGCTGCCTGGTCAAG | | CTTCTATCCCAGAGAGGCCAAAGT | |
| | GACTACTTCCCCGAACCGGTGACGG | | ACAGTGGAAGGTGGATAACGCCCT | |
| | TGTCGTGGAACTCAGGCGCCCTGAC | | CCAATCGGGTAACTCCCAGGAGAG | |
| | CAGCGGCGTGCACACCTTCCCGGCT | | TGTCACAGAGCAGGACAGCAAGGA | |
| | GTCCTACAGTCCTCAGGA | | CAGCACCTACAGCCTCAGCAGCAC | |
| | | | CCTGACGCTGAGCAAAGCAGACTA | |
| | | | CGAGAA | |
| S144-1827 | GAGGTGCAGCTGGTGGAGTCTGGGG | 1694 | GAAATTGTGTTGACGCAGTCTCCA | 1784 |
| | GAGACGTGGTCCAGCCTGGGGGGTC | | GGCACCCTGTCTTTGTCTCCAGGGG | |
| | CCTGAGACTCTCCTGTGCAGCCTCT | | AAAGAGCCACCCTCTCCTGCAGGG | |
| | GGAATTACCTTTAGTAACTATTGGA | | CCAGTCAGAGTATTAGCAACAGCT | |
| | TGACCTGGGTCCGCCAGGCTCCAGG | | ACTTAGTCTGGTACCAGCAGAAAC | |
| | GAAAGGGCTGGAGTGGGTGGCCAC | | CTGGCCAGGCTCCCAGGCTCCTCAT | |
| | CATAAAGAAGGATGGAGGGGAGCA | | CTATGGTGCATCCACCAGGGCCAC | |
| | GTACTATGTGGACTCTGTGAAGGGC | | TGGCATCCCAGACAGGTTCAGTGG | |
| | CGATTCACCATCTCCAGAGACAACG | | CAGTGGGTCTGGGACAGACTTCAC | |
| | CCAGGAATTCACTGTATCTACAAAT | | TCTCACCATCAGCAGACTGGAGCC | |
| | AAACAGCCTGAGGGCCGAGGATAC | | TGAAGATTTTGCAGTGTATTACTGT | |
| | GGCTGTCTATTACTGTGCGAGGGGT | | CAGCAGTATGGTAGCTCACCGTGG | |
| | GGATCTAGCAGCAGCTACTACTGGA | | ACGTTCGGCCAAGGGACCACGGTG | |
| | TCTACTGGGGCCAGGGAACCCTGGT | | GAAATCAAACGAACTGTGGCTGCA | |
| | CACCGTCTCCTCAGGGAGTGCATCC | | CCATCTGTCTTCATCTTCCCGCCAT | |
| | GCCCCAACCCTTTTCCCCCTCGTCTC | | CTGATGAGCAGTTGAAATCTGGAA | |
| | CTGTGAGAATTCCCCGTCGGATACG | | CTGCCTCTGTTGTGTGCCTGCTGAA | |
| | AGCAGCGTG | | TAACTTCTATCCCAGAGAGGCCAA | |
| | | | AGTACAGTGGAAGGTGGATAACGC | |
| | | | CCTCCAATCGGGTAACTCCCAGGA | |
| | | | GAGTGTCACAGAGCAGGACAGCAA | |
| | | | GGACAGCACCTACAGCCTCAGCAG | |
| | | | CACCCTGACGCTGAGCAAAGCAGA | |
| | | | CTACGAGAA | |
| S144-1848 | GAGGTGCAGCTGGTGGAGTCTGGGG | 1695 | CAGTCTGTGCTGACTCAGCCACCCT | 1785 |
| | GAGGCCTGGTCAAGCCTGGGGGGTC | | CAGCGTCTGGGACCCCCGGGCAGA | |
| | CCTGAGACTCTCCTGTGCAGCCTCT | | GGGTCACCATCTCTTGTTCTGGAAG | |
| | GGATTCACCTTCAGTAGCTATAGCA | | CAGCTCCAACATCGAACATAATTA | |
| | TGAACTGGGTCCGCCAGGCTCCAGG | | TGTATTCTGGTACCAGCAACTCCCA | |
| | GAAGGGGCTGGAGTGGGTCTCGTCC | | GGAACGGCCCCCAAACTCCTCATC | |
| | ATTAGTAGTAGTAGTAGTTACATAT | | TATAGTAATAATCACCGGCCCTCA | |
| | ACTACGCAGACTCAGTGAAGGGCCG | | GGGGTCCCTGACCGATTCTCTGGCT | |
| | ATTCACCATCTCCAGAGACAACGCC | | CCAAGTCTGGCACCTCAGCCTCCCT | |
| | AAGAATTCACTGTATCTGCAACTGA | | GGCCATCAGTGGGCTCCGGTCCGA | |
| | ACAGCCTGAGAGCCGAGGACACGG | | GGATGAGGCTGATTATTACTGTGC | |
| | CTGTGTACTACTGTGCGAGAGATCG | | AGCATGGGATGCCAGCCTGAGTGG | |
| | GGACCAGTTGATATTCTCGGCCGCT | | TCCTGTGGTATTCGCCGGAGGGAC | |
| | TTTGATATCTGGGGCCAAGGGACAA | | CAAGCTGACCGTCCTAGGTCAGCC | |
| | TGGTCACCGTCTCTTCAGCCTCCACC | | CAAGGCTGCCCCCTCGGTCACTCTG | |
| | AAGGGCCCATCGGTCTTCCCCCTGG | | TTCCCGCCCTCCTCTGAGGAGCTTC | |
| | CACCCTCCTCCAAGAGCACCTCTGG | | AAGCCAACAAGGCCACACTGGTGT | |
| | GGGCACAGCGGCCCTGGGCTGCCTG | | GTCTCATAAGTGACTTCTACCCGGG | |
| | GTCAAGGACTACTTCCCCGAACCGG | | AGCCGTGACAGTGGCCTGGAAGGC | |
| | TGACGGTGTCGTGGAACTCAGGCGC | | AGATAGCAGCCCCGTCAAGGCGGG | |
| | CCTGACCAGCGGCGTGCACACCTTC | | AGTGGAGACCACCACACCCTCCAA | |
| | CCGGCTGTCCTACAGTCCTCAGGA | | ACAAAGCAACAACAAGTACGCGGC | |
| | | | CAGCAGCTA | |
| S144-1850 | GAGGTGCAGCTGGTGGAGTCTGGGG | 1696 | GACATCCAGATGACCCAGTCTCCTT | 1786 |
| | GAGGCTTGGTACAGCCTGGGGGGTC | | CCACCCTGTCTGCATCTGTAGGAG | |
| | CCTGAGACTCTCCTGTGCAGCCTCT | | ACAGAGTCACCATCACTTGCCGGG | |
| | GGATTCACCTTTAGCAGCTATGCCA | | CCAGTCAGAGTATTACTAGCTGGTT | |
| | TGAGTTGGGTCCGCCAGGCTCCAGG | | GGCCTGGTATCAGCAGAAACCAGG | |
| | GAAGGGGCTGGAGTGGGTCTCAGCT | | GAAAGCCCCTAAGCTCCTGATCTA | |
| | ATTAGTGGTAGTGGTGGTAGCACAT | | TGATGCCTCCAATTTGGAAAGTGG | |
| | ACTACGCAGACTCCGTGAAGGGCCG | | GGTCCCATCAAGGTTCAGCGGCAG | |
| | GTTCACCATCTCCAGAGCCAATTCC | | TGGATCTGGGACAGAATTCACTCT | |
| | AAGAACACGCTGTATCTGCAAATGA | | CACCATCAGCAGCCTGCAGCCTGA | |
| | ACAGCCTGAGAGCCGAGGACACGG | | TGATTTTGCAACTTATTACTGCCAA | |
| | CCGTATATTACTGTGCGAAAGGCCC | | CAGTATAATAATTATCTGGGGACG | |
| | GCGCTTTAGTCGCGACTACTTTGAC | | TTCGGCCAAGGGACCAAGGTGGAA | |
| | TACTGGGGCCAGGGAACCCTGGTCA | | ATCAAACGAACTGTGGCTGCACCA | |
| | CCGTCTCCTCAGCCTCCACCAAGGG | | TCTGTCTTCATCTTCCCGCCATCTG | |
| | CCCATCGGTCTTCCCCCTGGCACCCT | | ATGAGCAGTTGAAATCTGGAACTG | |
| | CCTCCAAGAGCACCTCTGGGGGCAC | | CCTCTGTTGTGTGCCTGCTGAATAA | |
| | AGCGGCCCTGGGCTGCCTGGTCAAG | | CTTCTATCCCAGAGAGGCCAAAGT | |
| | GACTACTTCCCCGAACCGGTGACGG | | ACAGTGGAAGGTGGATAACGCCCT | |
| | TGTCGTGGAACTCAGGCGCCCTGAC | | CCAATCGGGTAACTCCCAGGAGAG | |
| | CAGCGGCGTGCACACCTTCCCGGCT | | TGTCACAGAGCAGGACAGCAAGGA | |
| | GTCCTACAGTCCTCAGGA | | CAGCACCTACAGCCTCAGCAGCAC | |
| | | | CCTGACGCTGAGCAAAGCAGACTA | |
| | | | CGAGAA | |
| S144-2234 | CAGGTCCAGCTGGTGCAATCTGGGG | 1697 | GACATCGTGATGACCCAGTCTCCA | 1787 |
| | CTGAGGTGAAGAAGCCTGGGTCCTC | | GACTCCCTGACTGTGTCTCTGGGCG | |
| | GGTGAAGGTCTCCTGCAAGGCTTCT | | AGAGGGCCACCATCAACTGCAAGT | |
| | GGAGGCACCTTCAGCAGATATACTA | | CCAGCCAGAGTGTTTTATACAGCTC | |
| | TCAGCTGGGTGCGACAGGCCCCTGG | | CAACAATAAGAACTACTTAGCTTG | |
| | ACAAGGGCTTGAGTGGATGGGAAG | | GTACCAGCAGAAACCAGGACAGCC | |
| | GATCATCCCTATACTTGGTACAGCA | | TCCTAAGCTGCTCATTTACTGGGCA | |
| | AACTACGCACAGAATTTCCAGGGCA | | TCTACCCGGGAATCCGGGGTCCCT | |
| | GAGTCACGATTACCGCGGACAAATC | | GACCGATTCAGTGGCAGCGGCTCT | |
| | CACGAGCACAGCCTACATGGAGCTG | | GGGACAGATTTCACTCTCACCGTC | |
| | AGTAGCCTGAGATCTGAGGACACGG | | AGCAGCCTGCAGGCTGAAGATGTG | |
| | CCGTGTATTACTGTGCGAGACACGG | | GCAGTTTATTACTGTCAGCAATATT | |
| | ATACAGCTATGGTCCCTTTGACTAC | | ATAGTACTCCTGGAACGTTCGGCC | |
| | TGGGGCCAGGGAACCCTGGTCACCG | | AAGGGACCAAGGTGGAAATCAAAC | |
| | TCTCCTCAGCCTCCACCAAGGGCCC | | GAACTGTGGCTGCACCATCTGTCTT | |
| | ATCGGTCTTCCCCCTGGCACCCTCCT | | CATCTTCCCGCCATCTGATGAGCAG | |
| | CCAAGAGCACCTCTGGGGGCACAGC | | TTGAAATCTGGAACTGCCTCTGTTG | |
| | GGCCCTGGGCTGCCTGGTCAAGGAC | | TGTGCCTGCTGAATAACTTCTATCC | |
| | TACTTCCCCGAACCGGTGACGGTGT | | CAGAGAGGCCAAAGTACAGTGGAA | |
| | CGTGGAACTCAGGCGCCCTGACCAG | | GGTGGATAACGCCCTCCAATCGGG | |
| | CGGCGTGCACACCTTCCCGGCTGTC | | TAACTCCCAGGAGAGTGTCACAGA | |
| | CTACAGTCCTCAGGAG | | GCAGGACAGCAAGGACAGCACCTA | |
| | | | CAGCCTCAGCAGCACCCTGACGCT | |
| | | | GAGCAAAGCAGACTACGAGAA | |
| S564-105 | CAGGTGCGGCTGCAGGAGTCGGGCC | 1698 | CAGTCTGCCCTGACTCAGCCTGCCT | 1788 |
| | CAGGACTGGTGAAGCCTTCACAGAC | | CCGTGTCTGGGTCTCCTGGACAGTC | |
| | CCTGTCCCTCACCTGCACTGTCTCTG | | GATCACCATCTCCTGCACTGGAAC | |
| | GTGGCTCCATCAGCAGTGGTAGTTA | | CAGCAGTGACGTTGGTGCTTATAA | |
| | CTACTGGAGCTGGATCCGGCAGCCC | | CTATGTCTCCTGGTACCAACAGCAC | |
| | GCCGGGAAGGGACTGGAGTGGATT | | CCAGGCAAAGCCCCCAAACTCATG | |
| | GGGCGTTTCCATACCAGTGGGAGCA | | ATTTATGAGGTCAGTAATCGGCCCT | |
| | CCAACTACAATCCCTCCCTCAAGAG | | CAGGGGTTTCTAATCGCTTCTCTGG | |
| | TCGAGTCACCATATCAGTAGACACG | | CTCCAAGTCTGGCAACACGGCCTC | |
| | TCCAAGAACCAGTTCTCCCTGAAGC | | CCTGACCATCTCTGGGCTCCAGGCT | |
| | TGAGTTCTGTGACCGCCGCAGACAC | | GAGGACGAGGCTGATTATTACTGC | |
| | GGCCGTGTATTACTGTGCGAGAGAT | | AGCTCATATACAAGCAGCACCTTC | |
| | TTAAAGGGAAAGACGTGGATACAG | | TTCGGAACTGGGACCACGGTCACC | |
| | ACCCCCTTTGACTACTGGGGCCAGG | | GTCCTAGGTCAGCCCAAGGCCAAC | |
| | GAATCCTGGTCACCGTCTCCTCAGC | | CCCACTGTCACTCTGTTCCCGCCCT | |
| | CTCCACCAAGGGCCCATCTGTCTTC | | CCTCTGAGGAGCTCCAAGCCAACA | |
| | CCCCTGGCACCCTCCTCCAAGAGCA | | AGGCCACACTAGTGTGTCTGATCA | |
| | CCTCTGGGGGCACAGCGGCCCTGGG | | GTGACTTCTACCCGGGAGCTGTGA | |
| | CTGCCTGGTCAAGGACTACTTCCCC | | CAGTGGCCTGGAAGGCAGATGGCA | |
| | GAACCGGTGACGGTGTCGTGGAACT | | GCCCCGTCAAGGCGGGAGTGGAGA | |
| | CAGGCGCTCTGACCAGCGGCGTGCA | | CCACCACACCCTCCAAACAAAGCA | |
| | CACCTTCCCGGCTGTCCTACAGTCCT | | ACAACAAGTACGCGGCCAGCAGCT | |
| | CAGGA | | AC | |
| S564- | GAGGTGCAGCTGGTGGAGTCTGGGG | 1699 | TCCTATGTGCTGACTCAGCCACCCT | 1789 |
| 14 | GAGGCTTGGTCCAGCCTGGGGGGTC | | CAGTGTCAGTGGCCCCAGGAAAGA | |
| | CCTGAGACTCTCCTGTGCAGCCTCT | | CGGCCAGGATTACCTGTGGGGGAA | |
| | GGACTCACCTTTAGTAGCTATTGGA | | ACAACATTGGAAGTAAAAGTGTGC | |
| | TGAGCTGGGCCCGCCAGGCTCCAGG | | ACTGGTACCAGCAGAGGCCAGGCC | |
| | GAAGGGGCTGGAGTGGGTGGCCAA | | AGGCCCCTGTACTGGTCATCTATTA | |
| | TATAAAGAAAGATGGAAGTGAGAA | | TGATAGCGACCGGCCCTCAGGGAT | |
| | ATACTATGTGGACTCTGTGAAGGGC | | CCCTGAGCGATTCTCTGGCTCCAAC | |
| | CGATTCACCATCTCCAGAGACAACG | | TCTGGGAACACGGCCACCCTGACC | |
| | CCAAGAACTCACTGTATCTGCAAAT | | ATCAGCAGGGTCGAGGCCGGGGAT | |
| | GAACAGCCTGAGAGTCGAGGACAC | | GAGGCCGACTATTACTGTCAGGTG | |
| | GGCTGTGTATTACTGTGCGAGTGAA | | TGGGATAGTAGTAGTGATCACCAT | |
| | CCTCCCCACTACGGTGGTAACTCCG | | TATGTCTTCGGAACTGGGACCAAG | |
| | GGGCTGAATACTTCCAGCACTGGGG | | GTCACCGTCCTAGGTCAGCCCAAG | |
| | CCAGGGCACCCTGGTCACCGTCTCC | | GCCAACCCCACTGTCACTCTGTTCC | |
| | TCAGCACCCACCAAGGCTCCGGATG | | CGCCCTCCTCTGAGGAGCTTCAAG | |
| | TGTTCCCCATCATATCAGGGTGCAG | | CCAACAAGGCCACACTGGTGTGTC | |
| | ACACCCAAAGGATAACAGCCCTGTG | | TCATAAGTGACTTCTACCCGGGAG | |
| | GTCCTGGCATGCTTGATAACTGGGT | | CCGTGACAGTGGCCTGGAAGGCAG | |
| | ACCACCC | | ATAGCAGCCCCGTCAAGGCGGGAG | |
| | | | TGGAGACCACCAAACCCTCCAAAC | |
| | | | AGAGCAACAACAAGTACGCGGCCA | |
| | | | GCAGCTA | |
| S564-68 | CAGGTGCAGCTGGTGCAGTCTGGGG | 1700 | CAGTCTGCCCTGACTCAGCCTCCCT | 1790 |
| | CTGAGGTGAAGAAGCCTGGGGCCTC | | CCGCGTCCGGGTCTCCTGGACAGT | |
| | AGTGAAGGTCTCCTGCAAGGCTTCT | | CAGTCACCATCTCCTGCACTGGAA | |
| | GGATACATCTTCACCGGCTATTATA | | CCAGCAGTGACGTTGGTGGTTATA | |
| | TGCACTGGGTGCGACAGGCCCCTGG | | ACTATGTCTCCTGGTACCAACAGC | |
| | ACAAGGGCTTGAGTGGATGGGATG | | ACCCAGGCAAAGCCCCCAAACTCA | |
| | GATCAACCCTAACAGTGGTGGCACT | | TGATTTATGAGGTCAGTAAGCGGC | |
| | AACTATGCACAGAAGTTTCAGGGCA | | CCTCAGGGGTCCCTGATCGCTTCTC | |
| | GGGTCACCATGACCAGGGACACGTC | | TGGCTCCAAGTCTGGCAACACGGC | |
| | CATCACCACAGCCTACATGGAGCTG | | CTCCCTGACCGTCTCTGGGCTCCAG | |
| | AGCAGGCTGAGATCTGACGACACG | | GCTGAGGATGAGGCTGATTATTTCT | |
| | GCCTTTTATTACTGTGCGAGAGTCA | | GCAGCTCATATGCAGACAGCAACA | |
| | AGAGGTTTTCGATTTTTGGAGTGGA | | ATTTGGTATTCGGCGGAGGGACCA | |
| | GCTTGACTACTGGGGCCAGGGAACC | | AGCTGACCGTCCTAGGTCAGCCCA | |
| | CTGGTCACCGTCTCCTCAGCCTCCA | | AGGCTGCCCCCTCGGTCACTCTGTT | |
| | CCAAGGGCCCATCGGTCTTCCCCCT | | CCCGCCCTCCTCTGAGGAGCTTCAA | |
| | GGCACCCTCCTCCAAGAGCACCTCT | | GCCAACAAGGCCACACTGGTGTGT | |
| | GGGGGCACAGCGGCCCTGGGCTGCC | | CTCATAAGTGACTTCTGCCCGGGA | |
| | TGGTCAAGGACTACTTCCCCGAACC | | GCCGTGACAGTGGCCTGGAAGGCA | |
| | GGTGACGGTGTCGTGGAACTCAGGC | | GATAGCAGCCCCGTCAAGGCGGGA | |
| | GCCCTGACCAGCGGCGTGCACACCT | | GTGGAGACCACCACACCCTCCAAA | |
| | TCCCGGCTGTCCTACAGTCCTCAGG | | CAAAGCAACAACAAGTACGCGGCC | |
| | A | | AGCAGCTACC | |
| S564-98 | CAGGTGCAGCTGCAGGAGTCGGGCC | 1701 | GACATCCAGATGACCCAGTCTCCA | 1791 |
| | CAGGACTGGTGAAGCCTTCGGAGAC | | TCCTCCCTGTCTGCATCTGTAGGAG | |
| | CCTGTCCCTCACCTGCACTGTCTCTG | | ACAGAGTCACCATCACTTGCCGGG | |
| | GTGGCTCCATCAGTAGTTACTACTG | | CAAGTCAGAGCATTCGCAGCTATT | |
| | GAGCTGGATCCGGCAGCCCCCAGGG | | TAAATTGGTATCAGCAGAAACCAG | |
| | AAGGGACTGGAGTGGATTGGGTATA | | GGAAAGCCCCTAAGCTCCTGATCT | |
| | TCTATTACAGTGGGAGCACCAACTA | | ATGCTGCATCCAGTTTGCAAAGTG | |
| | CAACCCCTCCCTCAAGAGTCGAGTC | | GCGTCCCATCAAGGTTCAGTGGCA | |
| | ACCATATCAGTAGACACGTCCAAGA | | GTGGATCTGGGACAGATTTCACTCT | |
| | ACCAGTTCTCCCTGAAGCTGAGCTC | | CACCATCGGCAGTCTGCAACCTGA | |
| | TGTGACCGCCGCAGACACGGCCGTG | | AGATTTTGCAACTTACTACTGTCAA | |
| | TATTACTGTGCGAGACATCAATCGC | | CAGAGTTACAGTACCTCCGTGGCG | |
| | GGTGGAATATAGTGGCTACGATGGA | | TTCGGCCAAGGGACCAAGGTGGAA | |
| | CTTTGACTACTGGGGCCAGGGAACC | | ATCAAACGAACTGTGGCTGCACCA | |
| | CTGGTCACCGTCTCCTCAGCCTCCA | | TCTGTCTTCATCTTCCCGCCATCTG | |
| | CCAAGGGCCCATCGGTCTTCCCCCT | | ATGAGCAGTTGAAATCTGGAACTG | |
| | GG | | CCTCTGTTGTGTGCCTGCTGAATAA | |
| | | | CTTCTATCCCAGAGAGGCCAAAGT | |
| | | | ACAGTGGAAGGTGGATAACGCCCT | |
| | | | CCAATCGGGTAACTCCCAGGAGAG | |
| | | | TGTCACAGAGCAGGACAGCAAGGA | |
| | | | CAGCACCTACAGCCTCAGCAGCAC | |
| | | | CCTGACGCTGAGCAAAGCAGACTA | |
| | | | CGAGA | |
| S564-105 | CAGGTGCGGCTGCAGGAGTCGGGCC | 1702 | CAGTCTGCCCTGACTCAGCCTGCCT | 1792 |
| | CAGGACTGGTGAAGCCTTCACAGAC | | CCGTGTCTGGGTCTCCTGGACAGTC | |
| | CCTGTCCCTCACCTGCACTGTCTCTG | | GATCACCATCTCCTGCACTGGAAC | |
| | GTGGCTCCATCAGCAGTGGTAGTTA | | CAGCAGTGACGTTGGTGCTTATAA | |
| | CTACTGGAGCTGGATCCGGCAGCCC | | CTATGTCTCCTGGTACCAACAGCAC | |
| | GCCGGGAAGGGACTGGAGTGGATT | | CCAGGCAAAGCCCCCAAACTCATG | |
| | GGGCGTTTCCATACCAGTGGGAGCA | | ATTTATGAGGTCAGTAATCGGCCCT | |
| | CCAACTACAATCCCTCCCTCAAGAG | | CAGGGGTTTCTAATCGCTTCTCTGG | |
| | TCGAGTCACCATATCAGTAGACACG | | CTCCAAGTCTGGCAACACGGCCTC | |
| | TCCAAGAACCAGTTCTCCCTGAAGC | | CCTGACCATCTCTGGGCTCCAGGCT | |
| | TGAGTTCTGTGACCGCCGCAGACAC | | GAGGACGAGGCTGATTATTACTGC | |
| | GGCCGTGTATTACTGTGCGAGAGAT | | AGCTCATATACAAGCAGCACCTTC | |
| | TTAAAGGGAAAGACGTGGATACAG | | TTCGGAACTGGGACCACGGTCACC | |
| | ACCCCCTTTGACTACTGGGGCCAGG | | GTCCTAGGTCAGCCCAAGGCCAAC | |
| | GAATCCTGGTCACCGTCTCCTCAGC | | CCCACTGTCACTCTGTTCCCGCCCT | |
| | CTCCACCAAGGGCCCATCTGTCTTC | | CCTCTGAGGAGCTCCAAGCCAACA | |
| | CCCCTGGCACCCTCCTCCAAGAGCA | | AGGCCACACTAGTGTGTCTGATCA | |
| | CCTCTGGGGGCACAGCGGCCCTGGG | | GTGACTTCTACCCGGGAGCTGTGA | |
| | CTGCCTGGTCAAGGACTACTTCCCC | | CAGTGGCCTGGAAGGCAGATGGCA | |
| | GAACCGGTGACGGTGTCGTGGAACT | | GCCCCGTCAAGGCGGGAGTGGAGA | |
| | CAGGCGCTCTGACCAGCGGCGTGCA | | CCACCACACCCTCCAAACAAAGCA | |
| | CACCTTCCCGGCTGTCCTACAGTCCT | | ACAACAAGTACGCGGCCAGCAGCT | |
| | CAGGA | | AC | |
| S564- | CAGGTGCAGCTGGTGCAGTCTGGGG | 1703 | CAGTCTGCCCTGACTCAACCTCCCT | 1793 |
| 134 | CTGAGGTGAAGAAGCCTGGGGCCTC | | CCGCGTCCGGGTCTCCTGGACAGT | |
| | AGTGAAGGTCTCCTGCAAGGCTTCT | | CAGTCACCATCTCCTGCACTGGAA | |
| | GGATACACCTTCACCGGCTACTATA | | CCAGCAGTGACGTTGGTGGTTATA | |
| | TGCACTGGGTGCGACAGGCCCCTGG | | ACTATGTCTCCTGGTACCAGCAAC | |
| | ACAAGGGCTTGAGTGGATGGGATG | | ACCCAGGCAAAGCCCCCAAACTCA | |
| | GATCAACCCTAACAGTGGTGGCACA | | TGATTTATGAGGTCAATAAGCGGC | |
| | AACTATGCACAGAAGTTTCAGGGCA | | CCTCAGGGGTCCCTGATCGCTTCTC | |
| | GGGTCACCATGACCAGGGACACGTC | | TGGCTCCAAGTCTGGCAACACGGC | |
| | CATCAACACAGCCTACATGGAGCTG | | CTCCCTGACCGTCTCTGGGCTCCAG | |
| | AGCAGGCTGAGATCTGACGACACG | | GCTGACGATGAGGCTGATTATTAC | |
| | GCCGTGTATTACTGTACGAGAGTCG | | TGCAGCTCATATGCAGGCAGCAAC | |
| | GGAGGTTTTCGATTTTTGGAGTGGA | | AATTTGGTTTTCGGCGGAGGGACC | |
| | GCTTGACTACTGGGGCCAGGGAACC | | AAGCTGACCGTCCTAGGTCAGCCC | |
| | CTGGTCACCGTCTCCTCAGCCTCCA | | AAGGCTGCCCCCTCGGTCACTCTGT | |
| | CCAAGGGCCCATCTGTCTTCCCCCT | | TCCCGCCCTCCTCTGAGGAGCTTCA | |
| | GGCACCCTCCTCCAAGAGCACCTCT | | AGCCAACAAGGCCACACTGGTGTG | |
| | GGGGGCACAGCGGCCCTGGGCTGCC | | TCTCATAAGTGACTTCTACCCGGGA | |
| | TGGTCAAGGACTACTTCCCCGAACC | | GCCGTGACAGTGGCCTGGAAGGCA | |
| | GGTGACGGTGTCGTGGAACTCAGGC | | GATAGCAGCCCCGTCAAGGCGGGA | |
| | GCCCTGACCAGCGGCGTGCACACCT | | GTGGAGACCACCACACCCTCCAAA | |
| | TCCCGGCTGTCCTACAGTCCTCAGG | | CAAAGCAACAACAAGTACGCGGCC | |
| | A | | AGCAGCTA | |
| S564-138 | CAGGTGCTCCTGGTGCAGTCTGGGG | 1704 | CAGTCTGCCCTGACTCAGCCTGCCT | 1794 |
| | CTGAGGTGAAGAAGCCTGGGGCCTC | | CCGTGTCTGGGTCTCCTGGACAGTC | |
| | AGTGAAGGTCTCCTGCAAGGCTTCT | | GATCACCATCTCCTGCACTGGAAC | |
| | GGATACACCTTCACCGGCTACTATC | | CAGCAGTGACGTTGGTGGTTATAA | |
| | TGCACTGGGTGCGACAGGCCCCTGG | | CTATGTCTCCTGGTACCAACAGCAC | |
| | ACAAGGGCTTGAGTGGATGGGATG | | CCAGGCAAAGCCCCCAAACTCATG | |
| | GATCAACCCTATCAGTGGTGGCACA | | ATTTATGAGGTCAGTAATCGGCCCT | |
| | AACTATGCACAGAATTTTCAGGACA | | CAGGGGTTTCTGATCGCTTCTCTGG | |
| | GGGTCACCATGACCAGGGACACGTC | | CTCCAAGTCTGGCAACACGGCCTC | |
| | CATCATCACAGCCTACATGGAACTG | | CCTGACCATCTCTGGGCTCCAGGCT | |
| | AGCAGGCTGAGATCTGACGACACG | | GAGGACGAGGCTGATTATTACTGC | |
| | GCCGTGTATTACTGTGCGAGACTTG | | AGCTCATATACAAGCAGCAGCACT | |
| | CCTATTATTATGATAGTAGTGCTTAC | | TATGTCTTCGGAACTGGGACCAAG | |
| | CGGGGTGCTTTTGATATCTGGGGCC | | GTCACCGTCCTAGGTCAGCCCAAG | |
| | AAGGGACAATGGTCACCGTCTCTTC | | GCCAACCCCACTGTCACTCTGTTCC | |
| | AGCCTCCACCAAGGGCCCATCTGTC | | CGCCCTCCTCTGAGGAGCTCCAAG | |
| | TTCCCCCTGGCACCCTCCTCCAAGA | | CCAACAAGGCCACACTAGTGTGTC | |
| | GCACCTCTGGGGGCACAGCGGCCCT | | TGATCAGTGACTTCTACCCGGGAG | |
| | GGGCTGCCTGGTCAAGGACTACTTC | | CTGTGACAGTGGCCTGGAAGGCAG | |
| | CCCGAACCGGTGACGGTGTCGTGGA | | ATGGCAGCCCCGTCAAGGCGGGAG | |
| | ACTCAGGCGCCCTGACCAGCGGCGT | | TGGAGACCACCAAACCCTCCAAAC | |
| | GCACACCTTCCCGGCTGTCCTACAG | | AGAGCAACAACAAGTACGCGGCCA | |
| | TCCTCAGG | | GCAGCTA | |
| S564-152 | CAGGTGCAGCTGGTGGAGTCTGGGG | 1705 | GACATCCAGATGACCCAGTCTCCA | 1795 |
| | GAGGCGTGGTCCAGCCTGGGAGGTC | | TCCTCCCTGTCTGCATCTGTAGGAG | |
| | CCTGAGACTCTCCTGTGCAGCGTCT | | ACAGAGTCACCATCACTTGCCAGG | |
| | GGATTCACCTTCAGTTACTATGGCA | | CGAGTCAGGACATTAACAACTATT | |
| | TGCACTGGGTCCGCCAGGCTCCAGG | | TAAATTGGTATCAGCAGAAACCAG | |
| | CAAGGGGCTGGAGTGGGTGGCAGTT | | GGAAAGCCCCTAAGCTCCTGATCT | |
| | ATATGGTATGATGGAAGTAATAAAC | | ACGATGCATCCAATTTGGAAACAG | |
| | ACTATGCAGACTCCGTGAAGGGCCG | | GGGTCCCATCAAGGTTCAGTGGGA | |
| | ATTCACCATCTCCAGAGACAATTCC | | GTGGATCTGGGACAGATTTTACTTT | |
| | AAGAACACGCTGTATCTGCAAATGA | | CACCATCAGCAGCCTGCAGCCTGA | |
| | ACAGCCTGAGAGCCGAGGACACGG | | AGATATTGCAACATATTACTGTCA | |
| | CTGTGTACTACTGTGCGAAAAATGC | | ACAGTATGACAATGTCCCTCCGCA | |
| | GGCCCCCTATTGTAGTGGTGGTAGC | | CACTTTTGGCCAGGGGACCAAGCT | |
| | TGCTACGGTACCTACTTTGACTACT | | GGAGATCAAACGAACTGTGGCTGC | |
| | GGGGCCAGGGAACCCTGGTCACCGT | | ACCATCTGTCTTCATCTTCCCGCCA | |
| | CTCCTCAGCCTCCACCAAGGGCCCA | | TCTGATGAGCAGTTGAAATCTGGA | |
| | TCTGTCTTCCCCCTGGCACCCTCCTC | | ACTGCCTCTGTTGTGTGCCTGCTGA | |
| | CAAGAGCACCTCTGGGGGCACAGC | | ATAACTTCTATCCCAGAGAGGCCA | |
| | GGCCCTGGGCTGCCTGGTCAAGGAC | | AAGTACAGTGGAAGGTGGATAACG | |
| | TACTTCCCCGAACCGGTGACGGTGT | | CCCTCCAATCGGGTAACTCCCAGG | |
| | CGTGGAACTCAGGCGCCCTGACCAG | | AGAGTGTCACAGAGCAGGACAGCA | |
| | CGGCGTGCACACCTTCCCGGCTGTC | | AGGACAGCACCTACAGCCTCAGCA | |
| | CTACAGTCCTCAGGA | | GCACCCTGACGCTGAGCAAAGCAG | |
| | | | ACTACGAGAA | |
| S564-218 | CAGGTCCAGCTGGTGCAGTCTGGGG | 1706 | CAGTCTGCCCTGACTCAGCCTCCCT | 1796 |
| | CTGAGGTGAAGAAGCCTGGGTCCTC | | CCGCGTCCGGGTCTCCTGGACAGT | |
| | GGTGAAGGTCTCCTGCAAGGCTTCT | | CAGTCACCATCTCCTGCACTGGAA | |
| | GGAGGCACCTTCAGCAGCTATGCTA | | CCAGCAGTGACGTTGGTGGTTATA | |
| | TCAGCTGGGTGCGACAGGCCCCTGG | | ACTATGTCTCCTGGTACCAACAGC | |
| | ACAAGGGCTTGAGTGGATGGGAGG | | ACCCAGGCAAAGCCCCCAAACTCA | |
| | GATCATCCCTATCTTTGGTACAGCA | | TGATTTATGAGGTCAGTAAGCGGC | |
| | AAGTACGCACAGAAGTTCCAGGGC | | CCTCAGGGGTCCCTGATCGCTTCTC | |
| | AGAGTCACGATTACCGCGGACGAAT | | TGGCTCCAAGTCTGGCAACACGGC | |
| | CCACGAGCACAGCCTACATGGAGCT | | CTCCCTGACCGTCTCTGGGCTCCAG | |
| | GAGCAGCCTGAGATCTGAGGACAC | | GCTGAGGATGAGGCTGATTATTAC | |
| | GGCCGTGTATTACTGTGCGAGAGGA | | TGCAGCTCATATGCAGGCAGCAAC | |
| | AAAGATGGCTACAATCCCTGGGGCG | | AATTTCGGGGTATTCGGCGGAGGG | |
| | CTTTTGATATCTGGGGCCAAGGGAC | | ACCAAGCTGACCGTCCTAGGTCAG | |
| | AATGGTCACCGTCTCTTCAGGGAGT | | CCCAAGGCTGCCCCCTCGGTCACTC | |
| | GCATCCGCCCCAACCCTTTTCCCCCT | | TGTTCCCGCCCTCCTCTGAGGAGCT | |
| | CGTCTCCTGTGAGAATTCCCCGTCG | | TCAAGCCAACAAGGCCACACTGGT | |
| | GATACGAGCAGCGTG | | GTGTCTCATAAGTGACTTCTACCCG | |
| | | | GGAGCCGTGACAGTGGCCTGGAAG | |
| | | | GCAGATAGCAGCCCCGTCAAGGCG | |
| | | | GGAGTGGAGACCACCACACCCTCC | |
| | | | AAACAAAGCAACAACAAGTACGCG | |
| | | | GCCAGCAGCTACCTGAGCCTGACG | |
| | | | CCTGAGCAGTGGAAGTCCCAC | |
| S564-249 | GAGGTGCAGCTGGTGGAGTCTGGGG | 1707 | CAGTCTGCCCTGACTCAGCCTGCCT | 1797 |
| | GAGGCTTGGTCCAGCCCGGGGGGTC | | CCGTGTCTGGGTCTCCTGGACAGTC | |
| | CCTGAGACTCTCCTGCGTAGCCTCT | | GATCACCATCTCCTGCACTGGAAC | |
| | GGATTCACCTTCAGTGACTATGCTA | | CAGCAGTGACATTGGTGGTTATAA | |
| | TGCACTGGGTCCGCCAGGCTCCAGG | | CTATGTCTCCTGGTACCAACAACAC | |
| | GAAGGGACTGGAATATATTGCAGCT | | CCAGGCAAAGCCCCCAAACTCATC | |
| | ATTAGTAGCAATGGGGGTAGGACAT | | ATTTCTGATGTCTCTAATCGGCCCT | |
| | ATTATGCAGACTCTGTGAAGGACAA | | CAGGGGTTTCTAGTCGCTTCTCTGG | |
| | ATTCACCATCTCCAGAGACAATTCC | | CTCCAAGTCTGGCAACACGGCCTC | |
| | AAGAACATCTTGTATCTTCACATGG | | CCTGACCATCTCTGGACTCCAGACT | |
| | GCAGCCTGAGAGCGGAGGACACGG | | GAGGACGAGGCTCATTATTATTGC | |
| | CTGTGTATTTCTGTGCGAGAGATCC | | AGCTCGTTTAGAAGTGGCATCACT | |
| | CCAGTCATGGGTGACTTCCACCACA | | CTCGGGGTATTCGGCGGGGGGACC | |
| | GCCCATTTCCAGCACTGGGGCCAGG | | AAGCTGACCGTCCTAGGTCAGCCC | |
| | GCACCCTGGTCACCGTCTCCTCAGC | | AAGGCTGCCCCCTCGGTCACTCTGT | |
| | ATCCCCGACCAGCCCCAAGGTCTTC | | TCCCGCCCTCCTCTGAGGAGCTTCA | |
| | CCGCTGAGCCTCTGCAGCACCCAGC | | AGCCAACAAGGCCACACTGGTGTG | |
| | CAGATGGGAACGTGGTCATCGCCTG | | TCTCATAAGTGACTTCTACCCGGGA | |
| | CCTGGTCCAGGGCTTCTTCCCCCAG | | GCCGTGACAGTGGCCTGGAAGGCA | |
| | GAGCCACTCAGTGTGACCTGGAGCG | | GATAGCAGCCCCGTCAAGGCGGGA | |
| | AAAGCGGACAGGGCGTGACCGCCA | | GTGGAGACCACCACACCCTCCAAA | |
| | GAAACTTCCC | | CAAAGCAACAACAAGTACGCGGCC | |
| | | | AGCAGCTA | |
| S564-265 | CAGGTGCAGCTGGTGCAGTCTGGGG | 1708 | CAGTCTGCCCTGACTCAGCCTCCCT | 1798 |
| | CTGAGGTGAAGAAGCCTGGGGCCTC | | CCGCGTCCGGGTCTCCTGGACAGT | |
| | AGTGAAGGTCTCCTGCAAGGCTTCT | | CAGTCACCATCTCCTGCACTGGAA | |
| | GGATACACCTTCACCGGCTACTATA | | CCAGCAGTGACGTTGGTGGTTATA | |
| | TGCACTGGGTGCGTCAGGCCCCTGG | | ACTTTGTCTCCTGGTACCAACAGCA | |
| | ACAAGGGCTTGAGTGGATGGGATG | | CCCAGGCAAAGCCCCCAAACTCAT | |
| | GATCAACCCTAACAGTGGTGCCATA | | GATTTATGAGGTCAGTAAGCGGCC | |
| | AACTATGCACAGAAGTTTCAGGGCA | | CTCAGGGGTCCCTGATCGCTTCTCT | |
| | GGGTCACCATGACCAGGGACACGTC | | GGCTCCAAGTCTGGCAACACGGCC | |
| | CATCAGCACAGCCTACATGGAGCTG | | TCCCTGACCGTCTCTGGGCTCCAGG | |
| | AGCAGCCTGAGATCTGACGACACGG | | CTGAGGATGAGGCTGATTATTACT | |
| | CCGTGTATTACTGTGCGAGAGTCGG | | GCAGCTCATATGGAGGCAGCAACA | |
| | GAGGTTTTCGATTTTTGGAGTGGAG | | ATTTGATATTCGGCGGAGGGACCA | |
| | CTTGATAACTGGGGCCAGGGAACCC | | GGCTGACCGTCCTAGGTCAGCCCA | |
| | TGGTCACCGTCTCCTCAGCCTCCAC | | AGGCTGCCCCCTCGGTCACTCTGTT | |
| | CAAGGGCCCATCTGTCTTCCCCCTG | | CCCGCCCTCCTCTGAGGAGCTTCAA | |
| | GCACCCTCCTCCAAGAGCACCTCTG | | GCCAACAAGGCCACACTGGTGTGT | |
| | GGGGCACAGCGGCCCTGGGCTGCCT | | CTCATAAGTGACTTCTACCCGGGA | |
| | GGTCAAGGACTACTTCCCCGAACCG | | GCCGTGACAGTGGCCTGGAAGGCA | |
| | GTGACGGTGTCGTGGAACTCAGGCG | | GATAGCAGCCCCGTCAAGGCGGGA | |
| | CCCTGACCAGCGGCGTGCACACCTT | | GTGGAGACCACCACACCCTCCAAA | |
| | CCCGGCTGTCCTACAGTCCTCAGGA | | CAAAGCAACAACAAGTACGCGGCC | |
| | | | AGCAGCTACCTGAGCCTGACGCCT | |
| | | | GAGCAGTGGAAGTCCCAC | |
| S564-275 | CAGGTGCAGCTGCAGGAGTCGGGCC | 1709 | GACATTCAGATGACCCAGTCTCCA | 1799 |
| | CAGGACTGGTGAAGCCTTCGGAGAC | | TCCTCCCTGTCTGCATCTATAGGAG | |
| | CCTGTCCCTCACCTGCACTGTCTCTG | | ACAGAGTCACCATCACTTGCCGGG | |
| | GTGGCTCCATCAGTAGTTACTACTG | | CAAGTCAGAGCATTAGCACCTATT | |
| | GAGCTGGATCCGGCAGCCCCCAGGG | | TAAATTGGTATCAGCAGAAACCAG | |
| | AAGGGACTGGAGTGGATTGGGTATA | | GGAAAGCCCCTAAACTCCTGATCT | |
| | TCTATTACAGTGGGAGCACCAAGTA | | ATGCTGCATCCAGTTTGCAAAGTG | |
| | CAACCCCTCCCTCAAGAGTCGAGTC | | GGGTCCCATCAAGGTTCAGTGGCA | |
| | ACCATATCAGTAGACACGTCCAAGA | | GTGGATCTGGGGCAGATTTCACTCT | |
| | AGCAGTTCTCCCTGAAGTTGAGCTC | | CACCATCAGCAGTCTGCAACCTGA | |
| | TGTGACCGCCGCAGACACGGCCGTG | | AGATTTTGCAACTTACTACTGTCAA | |
| | TATTACTGTGCGAGACATATAAAGA | | CAGAGTTACAGTACCCCGCTCACTT | |
| | TAGGAGTGGTCGGAGGCCTTACTTT | | TCGGCGGAGGGACGAAGGTGGAG | |
| | TGACTTCTGGGGCCAGGGAACCCTG | | ATCAAACGAACTGTGGCTGCACCA | |
| | GTCACCGTCTCCTCAGGGAGTGCAT | | TCTGTCTTCATCTTCCCGCCATCTG | |
| | CCGCCCCAACCCTTTTCCCCCTCGTC | | ATGAGCAGTTGAAATCTGGAACTG | |
| | TCCTGTGAGAATTCCCCGTCGGATA | | CCTCTGTTGTGTGCCTGCTGAATAA | |
| | CGAGCAGCGTG | | CTTCTATCCCAGAGAGGCCAAAGT | |
| | | | ACAGTGGAAGGTGGATAACGCC | |
| S564-287 | CAGGTGCAGCTGGTGCAGTCTGGGG | 1710 | CAGTCTGCCCTGACTCAGCCTGCCT | 1800 |
| | CTGAGGTGAAGAAGCCTGGGGCCTC | | CCGTGTCTGGGTCTCCTGGACAGTC | |
| | AGTGAAGGTCTCCTGCAAGGCTTCT | | GATCACCATCTCCTGCACTGGAAC | |
| | GGATACACCTTCACCGGCTACTATA | | CAGCAGTGACGTTGGTGGTTATAA | |
| | TGCACTGGGTGCGACAGGCCCCTGG | | CTATGTCTCCTGGTACCAACAACAC | |
| | ACAAGGGCTTGAGTGGATGGGATG | | CCAGGCAAAGCCCCCAAACTCATG | |
| | GATCAACCCTAACAGTGGTGGCACA | | ATTTATGATGTCAGTAATCGGCCCT | |
| | AACTATGCACAGAAGTTTCAGGGCA | | CAGGGGTTTCTAATCGCTTCTCTGG | |
| | GGGTCACCATGACCAGGGACACGTC | | CTCCAAGTCTGGCAACACGGCCTC | |
| | CATCAGCACAGCCTACATGGAGCTG | | CCTGACCATCTCTGGGCTCCAGGCT | |
| | AGCAGGCTGAGATGTGACGACACG | | GAGGACGAGGCTGATTATTACTGC | |
| | GCCGTGTATTACTGTGCGAGAGCCT | | AGCTCATATGCAAGCAGCAGCACT | |
| | CAACTCCGTATAGCAGTGGCTCCTG | | TGGGTGTTCGGCGGAGGGACCAAG | |
| | GGCGGACTACTGGGGCCAGGGAAC | | CTGACCGTCCTAGGTCAGCCCAAG | |
| | CCTGGTCACCGTCTCCTCAGGGAGT | | GCTGCCCCCTCGGTCACTCTGTTCC | |
| | GCATCCGCCCCAACCCTTTTCCCCCT | | CGCCCTCCTCTGAGGAGCTTCAAG | |
| | CGTCTCCTGTGAGAATTCCCCGTCG | | CCAACAAGGCCACACTGGTGTGTC | |
| | GATACGAGCAGCGTG | | TCATAAGTGACTTCTACCCGGGAG | |
| | | | CCGTGACAGTGGCCTGGAAGGCAG | |
| | | | ATAGCAGCCCCGTCAAGGCGGGAG | |
| | | | TGGAGACCACCACACCCTCCAAAC | |
| | | | AAAGCAACAACAAGTACGCGGCCA | |
| | | | GCAGCTATCTGAGCCTGACGCC | |
| S116-2822 | CAGGTGCAGCTGGTGGAGTCTGGGG | 2707 | GACATCCAGATGACCCAGTCTCCTT | 2756 |
| | GAGGCGTGGTCCAGCCTGGGAGGTC | | CCACCCTGTCTGCATCTGTAGGAG | |
| | CCTGAGACTCTCCTGTGCAGCCTCT | | ACAGAGTCACCATCACTTGCCGGG | |
| | GGATTCACCTTCAGTAGCTATGGCA | | CCAGTCAGAGTATTAGTAGCTGGT | |
| | TGCACTGGGTCCGCCAGGCTCCAGG | | TGGCCTGGTATCAGCAGAAACCAG | |
| | CAAGGGGCTGGAGTGGGTGGCAGTT | | GGAAAGCCCCTAAGCTCCTGATCT | |
| | ATATCATATGATGGAAGTAATAAAT | | ATGATGCCTCCAGTTTGGAAAGTG | |
| | ACTATGCAGACTCCGTGAAGGGCCG | | GGGTCCCATCAAGGTTCAGCGGCA | |
| | ATTCACCATCTCCAGAGACAATTCC | | GTGGATCTGGGACAGAATTCACTC | |
| | AAGAACACGCTGTATCTGCAAATGA | | TCACCATCAGCAGCCTGCAGCCTG | |
| | ACAGCCTGAGAGCTGAGGACACGG | | ATGATTTTGCAACTTATTACTGCCA | |
| | CTGTGTATTACTGTGCGAAAGGGGA | | ACAGTATAATAGTTATTCTCAAACT | |
| | TTACTATGGTTCGGGGAGTCAGTAC | | TTTGGCCAGGGGACCAAGCTGGAG | |
| | TACTTTGACTACTGGGGCCAGGGAA | | ATCAAACGAACTGTGGCTGCACCA | |
| | CCCTGGTCACCGTCTCCTCAGGGAG | | TCTGTCTTCATCTTCCCGCCATCTG | |
| | TGCATCCGCCCCAACCCTTTTCCCCC | | ATGAGCAGTTGAAATCTGGAACTG | |
| | TCGTCTCCTGTGAGAATTCCCCGTC | | CCTCTGTTGTGTGCCTGCTGAATAA | |
| | GGATACGAGCAGCGTG | | CTTCTATCCCAGAGAGGCCAAAGT | |
| | | | ACAGTGGAAGGTGGATAACGC | |
| S116-2825 | GAGGTGCAGCTGGTGGAGTCCGGG | 2708 | TCTTCTGAGCTGACTCAGGACCCTG | 2757 |
| | GGAGGCTTAGTTCAGCCTGGGGGGT | | CTGTGTCTGTGGCCTTGGGACAGA | |
| | CCCTGAGACTCTCCTGTGCAGCCTC | | CAGTCAGGATCACATGCCAAGGAG | |
| | TGGATTCACCTTCAGTAGCTACTGG | | ACAGCCTCAGAAGCTATTATGCAA | |
| | ATGCACTGGGTCCGCCAAGCTCCAG | | GCTGGTACCAGCAGAAGCCAGGAC | |
| | GGAAGGGGCTGGTGTGGGTCTCACG | | AGGCCCCTGTACTTGTCATCTATGG | |
| | TATTAATAGTGATGGGAGTAGCACA | | TAAAAACAACCGGCCCTCAGGGAT | |
| | AGCTACGCGGACTCCGTGAAGGGCC | | CCCAGACCGATTCTCTGGCTCCAGC | |
| | GATTCACCATCTCCAGAGACAACGC | | TCAGGAAACACAGCTTCCTTGACC | |
| | CAAGAACACGCTGTATCTGCAAATG | | ATCACTGGGGCTCAGGCGGAAGAT | |
| | AACAGTCTGAGAGCCGAGGACACG | | GAGGCTGACTATTACTGTAACTCCC | |
| | GCTGTGTATTACTGTGCAAGAGTCG | | GGGACAGCAGTGGTAACCTCGTGG | |
| | TTCTTACGTATTACTATGATAGTAGT | | TATTCGGCGGAGGGACCAAGCTGA | |
| | GGTTATCAGAATGCTTTTGATATCT | | CCGTCCTAGGTCAGCCCAAGGCTG | |
| | GGGGCCAAGGGACAATGGTCACCG | | CCCCCTCGGTCACTCTGTTCCCGCC | |
| | TCTCTTCAGGGAGTGCATCCGCCCC | | CTCCTCTGAGGAGCTTCAAGCCAA | |
| | AACCCTTTTCCCCCTCGTCTCCTGTG | | CAAGGCCACACTGGTGTGTCTCAT | |
| | AGAATTCCCCGTCGGATACGAGCAG | | AAGTGACTTCTACCCGGGAGCCGT | |
| | CGTG | | GACAGTGGCCTGGAAGGCAGATAG | |
| | | | CAGCCCCGTCAAGGCGGGAGTGGA | |
| | | | GACCACCAAACCCTCCAAACAGAG | |
| | | | CAACAACAAGTACGCGGCCAGCAG | |
| | | | CTA | |
| S116-3179 | CAGGTGCAGCTGCAGGAGTCGGGCC | 2709 | GACATCCAGATGACCCAGTCTCCA | 2758 |
| | CAGGACTGGTGAAGCCTTCGGAGAC | | TCTTCCGTGTCTGCATCTGTAGGAG | |
| | CCTGTCCCTCACCTGCACTGTCTCTG | | ACAGAGTCACCATCACTTGTCGGG | |
| | GTGGCTCCATCAGTAGTTACTACTG | | CGAGTCAGGGTATTAGCAGCTGGT | |
| | GAGCTGGATCCGGCAGCCCCCAGGG | | TAGCCTGGTATCAGCAGAAACCAG | |
| | AAGGGACTGGAGTGGATTGGGTATA | | GGAAAGCCCCTAAGCTCCTGATCT | |
| | TCTATTACAGTGGGAGCACCAACTA | | ATGCTGCATTCAGTTTGCAAAGTG | |
| | CAACCCCTCCCTCAAGAGTCGAGTC | | GGGTCCCATCAAGGTTCAGCGGCA | |
| | ACCATATCAGTAGACACGTCCAAGA | | GTGGATCTGGGACAGATTTCACTCT | |
| | ACCAGTTCTCCCTGAAGCTGACCTC | | CACCATCAGCAGCCTGCAGCCTGA | |
| | TGTGACCGCTGCGGACACGGCCGTG | | AGATTTTGCAACTTACTATTGTCAA | |
| | TATTACTGTGCGAGATGTGCCTTAC | | CAGGCTAACAGTTTCCCGCGGGGG | |
| | TACTAGGGAACGCTTTTGATATCTG | | CTCTCTTTCGGCGGAGGGACCAAG | |
| | GGGCCAAGGGACAATGGTCACCGTC | | GTGGAGATCAAACGAACTGTGGCT | |
| | TCTTCAGCTTCCACCAAGGGCCCAT | | GCACCATCTGTCTTCATCTTCCCGC | |
| | CGGTCTTCCCCCTGGCGCCCTGCTCC | | CATCTGATGAGCAGTTGAAATCTG | |
| | AGGAG | | GAACTGCCTCTGTTGTGTGCCTGCT | |
| | | | GAATAACTTCTATCCCAGAGAGGC | |
| | | | CAAAGTACAGTGGAAGGTGGATAA | |
| | | | CGC | |
| S144-121 | GAGGTGCACCTGTTGGAGTCTGGGG | 2710 | GAAATTGTGTTGACGCAGTCTCCA | 2759 |
| | GAGGCCTGGTACAGCCTGGGGGGTC | | GGCACCCTGTCGTTGTCTCCAGGA | |
| | CCTGAGACTCTCCTGTGCAGCCTCT | | GAAAGAGCCACCCTCTCCTGCAGG | |
| | GGATTCACCTTCAGCAGCTATGCCA | | GCCAGTCAGAGTGTTAGCAGCAGC | |
| | TGAGCTGGGTCCGCCAGACTCCAGG | | CACTTAGCCTGGTACCAGCAGAAA | |
| | GAAGGGGCTGGAGTGGATCTCAGCT | | CCTGGCCAGTCTCCCAGGCTCCTCA | |
| | ATTACTGCCAGTGGTTCTGACACAT | | TCTATGGTACATCCAACAGGGCCA | |
| | TCCACGCTGACTCCGTGAAGGGCCG | | CTGGCATCCCAGACAGGTTCAGTG | |
| | GTTCACCATCTCCAGAGACAATTCC | | GCAGTGGGTCTGGGACAGACTTCA | |
| | AAGGACACACTGTATCTGCAAATGA | | CTCTCAGCATCAGCAGACTGGAGC | |
| | ACAGCCTGAGAGTCGAGGACACGG | | CTGAAGATTTTGCAGTGTATTACTG | |
| | CCATATATTACTGTGCGAAAGGCTC | | TCAAGAATATGGTAGCTCACGGAT | |
| | TTCCACCGCCCGCCCCTACTACTTTG | | GTTCGGCCAAGGGACCAAGGTGGA | |
| | ACTACTGGGGCCAGGGAACCCTGGT | | AATCAAACGAACTGTGGCTGCACC | |
| | CACCGTCTCCTCAGGGAGTGCATCC | | ATCTGTCTTCATCTTCCCGCCATCT | |
| | GCCCCAACCCTTTTCCCCCTCGTCTC | | GATGAGCAGTTGAAATCTGGAACT | |
| | CTGTGAGAATTCCCCGTCGGATACG | | GCCTCTGTTGTGTGCCTGCTGAATA | |
| | AGCAGCGTG | | ACTTCTATCCCAGAGAGGCCAAAG | |
| | | | TACAGTGGAAGGTGGATAACGCCC | |
| | | | TCCAATCGGGTAACTCCCAGGAGA | |
| | | | GTGTCACAGAGCAGGACAGCAAGG | |
| | | | ACAGCACCTACAGCCTCAGCAGCA | |
| | | | CCCTGACGCTGAGCAAAGCAGACT | |
| | | | ACGAG | |
| S144-1364 | GAGGTGCAGCTGGTGCAGTCTGGAG | 2711 | GAAATTGTGTTGACGCAGTCTCCA | 2760 |
| | CAGAGATGAAAAAGCCCGGGGAGT | | GGCACCCTGTCTTTGTCTCCAGGGG | |
| | CTCTGAAGATCTCCTGTAAGGCTTC | | AAAGAGCCACCCTCTCCTGCAGGG | |
| | TGGATACTACTTTCCCAGCTACTGG | | CCAGTCAGGGTGTTAGCAGCAACT | |
| | ATCGCCTGGGTGCGCCAGATGCCCG | | ACTTAGCCTGGTACCAGCAGAAAC | |
| | GGAGAGGCCTGGAATGGATGGGGA | | CTGGCCAGGCTCCCAGGCTCCTCAT | |
| | TCATTTATCCTGTTGACTCTGAGACC | | CTATGGTGCATCCAGCAGGGCCAC | |
| | ACATACAGCCCGTCCTTCCAAGGCC | | TGGCATCCCAGACAGGTTCAGTGG | |
| | ACGTCACCATCTCAGCCGACAAGTC | | CAGTGGGTCTGGGACAGACTTCAC | |
| | CATCAGCACCGCCTACCTGCAGTGG | | TCTCACCATCAGCAGACTGGAGCC | |
| | AGCAGCCTGAAGGCCTCGGACACCG | | TGAAGATTTTGCAGTGTATTACTGT | |
| | CCATGTATTACTGTGCGAGACCGAA | | CAGCAGTATGGTACCACACCTAAT | |
| | TTACTATGGTTCGGGGAGCCCCCCG | | ACTTTCGGCGGAGGGACCAAGGTG | |
| | GGCTACTGGGGCCAGGGAACCCTGG | | GAGATCAAACGAACTGTGGCTGCA | |
| | TCACCGTCTCCTCAGGGAGTGCATC | | CCATCTGTCTTCATCTTCCCGCCAT | |
| | CGCCCCAACCCTTTTCCCCCTCGTCT | | CTGATGAGCAGTTGAAATCTGGAA | |
| | CCTGTGAGAATTCCCCGTCGGATAC | | CTGCCTCTGTTGTGTGCCTGCTGAA | |
| | GAGCAGCGTGGCCGTTGGCTG | | TAACTTCTATCCCAGAGAGGCCAA | |
| | | | AGTACAGTGGAAGGTGGATAACGC | |
| | | | CCTCCAATCGGGTAACTCCCAGGA | |
| | | | GAGTGTCACAGAGCAGGACAGCAA | |
| | | | GGACAGCACCTACAGCCTCAGCAG | |
| | | | CACCCTGACGCTGAGCAAAGCAGA | |
| | | | CTACGAGAA | |
| S144-292 | GAGGTGCAGCTGGTGCAGTCTGGAG | 2712 | GACATCCAGATGACCCAGTCTCCTT | 2761 |
| | CAGAGGTGAAAAAGCCCGGGGAGT | | CCACCCTGTCTGCATCTGTAGGAG | |
| | CTCTGAAGATCTCCTGTAAGGGTTC | | ACAGAGTCACCATCACTTGCCGGG | |
| | TGGATACACCTTTACCAACTACTGG | | CCAGTCAGAGTATTAGTAGCTGGT | |
| | ATCGGCTGGGTGCGCCAGATGCCCG | | TGGCCTGGTATCAGCAGAAACCAG | |
| | GGAAAGGCCTGGAGTGGATGGGGA | | GGAAAGCCCCTAACCTCCTGATCT | |
| | TCATCTATCCTGGTGACTCGGATAC | | ATGATGCCTCCAGTTTGGAAAGTG | |
| | CAGATACAGCCCGTCCTTCCAAGGC | | GGGTCCCATCAAGGTTCAGCGGCA | |
| | CAGGTCACCATCTCAGCCGACAAGT | | GTGGATCTGGGACAGAATTCACTC | |
| | CCATCAGCACCGCCTACCTGCAGTG | | TCACCATCAGCAGCCTGCAGCCTG | |
| | GAGCAGCCTGAAGGCCTCGGACACC | | ATGATTTTGCAACTTATTACTGCCA | |
| | GCCATGTATTACTGTGCGAGACTGT | | ACAGTATAATACTTACCCAAGGAC | |
| | TTTGTGGTGGTGACTGCCCGTTTGA | | GTTCGGCCAAGGGACCAAGGTGGA | |
| | CTACTGGGGCCAGGGAACCCTGGTC | | AATCAAACGAACTGTGGCTGCACC | |
| | ACCGTCTCCTCAGCCTCCACCAAGG | | ATCTGTCTTCATCTTCCCGCCATCT | |
| | GCCCATCGGTCTTCCCCCTGGCGCC | | GATGAGCAGTTGAAATCTGGAACT | |
| | CTGCTCCAGGAGCACCTCTGGGGGC | | GCCTCTGTTGTGTGCCTGCTGAATA | |
| | ACAGCGGCCCTGGGCTGCCTGGTCA | | ACTTCTATCCCAGAGAGGCCAAAG | |
| | AGGACTACTTCCCCGAACCGGTGAC | | TACAGTGGAAGGTGGATAACGCCC | |
| | GGTGTCGTGGAACTCAGGCGCCCTG | | TCCAATCGGGTAACTCCCAGGAGA | |
| | ACCAGCGGCGTGCACACCTTCCCGG | | GTGTCACAGAGCAGGACAGCAAGG | |
| | CTGTCCTACAGTCCTCAGGA | | ACAGCACCTACAGCCTCAGCAGCA | |
| | | | CCCTGACGCTGAGCAAAGCAGACT | |
| | | | ACGAGAA | |
| S155-37 | GAGGTGCAACTGTTGGAGTCTGGGG | 2713 | GAAATTGTGTTGACGCAGTCTCCA | 2762 |
| | GAGGCTTGGTGCAGCCGGGAGGGTC | | GGCACCCTGTCTTTGTCTCCAGGAG | |
| | CCTGAGACTCTCCTGCGCAGCCTCT | | AAAGAGCCACCCTCTCCTGCAGGG | |
| | GGATTCAGCTTTAGCAACTACGCCA | | CCAGTCAGACTGTTAGCAGCAACT | |
| | TGAGCTGGGTCCGCCAGGCTCCAGG | | ACTTAGCCTGGTACCAGCAGAAAC | |
| | GAAGGGGCTGGAGTGGGTCTCAGCT | | CTGCCCAGGGTCCCAGGCTCGTCA | |
| | GTTTCTGGTAATGGAGTTGGCACAT | | TCTATGGTGCATCCAACAGGGCCA | |
| | TCCACGCAGACTCCGTGAAGGGCCG | | CTGGCATCCCAGACAGGTTCAGTG | |
| | CTTCACCATCTCCAGAGACAATTCC | | GCAGTGGGTCTGGGACAGACTTCA | |
| | AAGGACACGTTCTATTTGCAAATGA | | CTCTCACCATCAGCAGACTGGAGC | |
| | GTGGCCTCACAGTCGACGACACGGC | | CTGAAGATTTTGCAGTGTATTACTG | |
| | CCTATATTATTGTGTGAAGGGAAGT | | TCAGCAGTATGGTAATTCAAGGAT | |
| | GCAGCCGCTCGCCCCTACTACTTTG | | TTTCGGCCAAGGGACCAAGGTGGA | |
| | ACTACTGGGGCCAGGGAATCCTGGT | | GATCAAACGAACTGTGGCTGCACC | |
| | CGCCGTCTCCTCAGGGAGTGCATCC | | ATCTGTCTTCATCTTCCCGCCATCT | |
| | GCCCCAACCCTTTTCCCCCTCGTCTC | | GATGAGCAGTTGAAATCTGGAACT | |
| | CTGTGAGAATTCCCCGTCGGATACG | | GCCTCTGTTGTGTGCCTGCTGAATA | |
| | AGCAGCGTG | | ACTTCTATCCCAGAGAGGCCAAAG | |
| | | | TACAGTGGAAGGTGGATAACGC | |
| S166-1318 | GAGGTGCAGCTGGTGGAGTCTGGGG | 2714 | TCCTATGAGCTGACTCAGCCACCCT | 2763 |
| | GAGGCTTGGTCCAGCCTGGGGGGTC | | CAGTGTCCGTGTCCCCAGGACAGA | |
| | CCTGAGACTCTCCTGTGCAGCCTCT | | CAGCCAGCATCACCTGCTCTGGAG | |
| | GGATTCACCTTTACTATCTATTGGAT | | ATAAATTGGGGGATAAATATGCTT | |
| | GAGCTGGGTCCGCCAGGCTCCAGGG | | GCTGGTATCAGCAGAAGCCAGGCC | |
| | AAGGGGCTGGAGTGGGTGGCCAAC | | AGTCCCCTGTGTTGGTCATCTATCA | |
| | ATAAAGCAAGATGGAAGTGAGAAA | | AGATAGCAAGCGGCCCTCAGGGAT | |
| | TACTATGTGGACTCTGTGAAGGGCC | | CCCTGAGCGATTCTCTGGCTCCAAC | |
| | GATTCACCATCTCCAGAGACAACGC | | TCTGGGAACACAGCCACTCTGACC | |
| | CAAGAATTCACTGTATCTGCAAATG | | ATCAGCGGGACCCAGGCTATGGAC | |
| | AACAGCCTGAGAGCCGAGGACACG | | GAGGCTGACTATTACTGTCAGGCG | |
| | GCCGTGTATTACTGTGCGAGAGATG | | TGGGACAGCAGCACCGTGGTATTC | |
| | GTATAGCAGTGGCTGGTGGGTTTGA | | GGCGGAGGGACCAAGCTGACCGTC | |
| | CTACTGGGGCCAGGGAACCCTGGTC | | CTAGGTCAGCCCAAGGCTGCCCCC | |
| | ACCGTCTCCTCAGGGAGTGCATCCG | | TCGGTCACTCTGTTCCCGCCCTCCT | |
| | CCCCAACCCTTTTCCCCCTCGTCTCC | | CTGAGGAGCTTCAAGCCAACAAGG | |
| | TGTGAGAATTCCCCGTCGGATACGA | | CCACACTGGTGTGTCTCATAAGTG | |
| | GCAGCGTG | | ACTTCTACCCGGGAGCCGTGACAG | |
| | | | TGGCCTGGAAGGCAGATAGCAGCC | |
| | | | CCGTCAAGGCGGGAGTGGAGACCA | |
| | | | CCACACCCTCCAAACAAAGCAACA | |
| | | | ACAAGTACGCGGCCAGCAGCTA | |
| S166-1366 | CAGATCACCTTGAAGGAGTCTGGTC | 2715 | TCCTATGAGCTGACTCAGCCACCCT | 2764 |
| | CTACGCTGGTGAAACCCACACAGAC | | CAGTGTCCGTGTCCCCAGGACAGA | |
| | CCTCACGCTGACCTGCACCTTCTCTG | | CAGCCAGCATCACCTGCTCTGGAG | |
| | GGTTCTCACTCAGCACTAGTGGAGT | | ATAAATTGGGGGATAAATATGCTT | |
| | GGGTGTGGGCTGGATCCGTCAGCCC | | GCTGGTATCAGCAGAAGCCAGGCC | |
| | CCAGGAAAGGCCCTGGAGTGGCTTG | | AGTCCCCTGTGCTGGTCATCTATCA | |
| | CACTCATTTATTGGGATGATGATAA | | AGATAGCAAGCGGCCCTCAGGGAT | |
| | GCGCTACAGGCCATCTCTGAAGAGC | | CCCTGAGCGATTCTCTGGCTCCAAC | |
| | AGGCTCAGCATCACCAAGGACACCT | | TCTGGGAACACAGCCACTCTGACC | |
| | CCAAAAACCAGGTGGTCCTTACAAT | | ATCAGCGGGACCCAGGCTATGGAT | |
| | GACCAACATGGACCCTGTGGACACA | | GAGGCTGACTATTACTGTCAGGCG | |
| | GCCACATATTACTGTGCACACCATC | | TGGGACAGCAGCACTAGGGATTAT | |
| | ACCCCATACTTGATTTTGACTACTG | | GTCTTCGGAACTGGGACCAAGGTC | |
| | GGGCCAGGGAACCCTGGTCACCGTC | | ACCGTCCTAGGTCAGCCCAAGGCC | |
| | TCCTCAGGGAGTGCATCCGCCCCAA | | AACCCCACTGTCACTCTGTTCCCGC | |
| | CCCTTTTCCCCCTCGTCTCCTGTGAG | | CCTCCTCTGAGGAGCTCCAAGCCA | |
| | AATTCCCCGTCGGATACGAGCAGCG | | ACAAGGCCACACTAGTGTGTCTGA | |
| | TG | | TCAGTGACTTCTACCCGGGAGCTGT | |
| | | | GACAGTGGCCTGGAAGGCAGATGG | |
| | | | CAGCCCCGTCAAGGCGGGAGTGGA | |
| | | | GACCACCAAACCCTCCAAACAGAG | |
| | | | CAACAACAAGTACGCGGCCAGCAG | |
| | | | CTA | |
| S166-2395 | CAGGTGCAGCTGCAGGAGTCGGGCC | 2716 | TCCTATGTGCTGACTCAGACACCCT | 2765 |
| | CAGGACTGGTGAAGCCTTCGGAGAC | | CGGTGTCAGTGGCCCCAGGACAGA | |
| | CCTGTCCCTCACCTGCACTGTCTCTG | | CGGCCAGGATTACCTGTGGGGGAA | |
| | GTGGCTCCATCAGTACTTACTACTG | | ACAACATTGGAAGTAAAAGTGTGC | |
| | GAGCTGGATCCGGCAGCCCGCCGGG | | ACTGGTACCAGCAGAAGCCAGGCC | |
| | AAGGGACTGGAGTGGATTGGGCGT | | AGGCCCCTGTGCTGGTCGTCCATG | |
| | ATCTATACCAGTGGGAGCACCAACT | | ATGAAAGCGACCGGCCCTCAGGGA | |
| | ACAACCCCTCCCTCAAGAGTCGGGT | | TCCCTGAGCGATTTTTTGGCTCCAA | |
| | CACCATGTCAGTAGACACGTCCAAG | | CTCTGGGAACACGGCCACCCTGAC | |
| | AACCAGTTCTCCCTGAAGCTGAGCT | | CATCAGCAGGGTCGAAGCCGGGGA | |
| | CTGTGACCGCCGCGGACACGGCCGT | | TGAGGCCGACTATTACTGTCAGGT | |
| | GTATTACTGTGCGAGAGAGGTTACT | | GTGGGATAGTAGTAGTGATCATCT | |
| | ATGATAGTACTGGGATACAACTGGT | | TCATGTCTTCGGAACTGGGACCAA | |
| | TCGACCCCTGGGGCCAGGGAACCCT | | GGTCACCGTCCTAGGTCAGCCCAA | |
| | GGTCACCGTCTCCTCTGCACCCACC | | GGCCAACCCCACTGTCACTCTGTTC | |
| | AAGGCTCCGGATGTGTTCCCCATCA | | CCGCCCTCCTCTGAGGAGCTCCAA | |
| | TATCAGGGTGCAGACACCCAAAGG | | GCCAACAAGGCCACACTAGTGTGT | |
| | ATAACAGCCCTGTGGTCCTGGCATG | | CTGATCAGTGACTTCTACCCGGGA | |
| | CTTGATAACTGGGTACCACC | | GCTGTGACAGTGGCCTGGAAGGCA | |
| | | | GATGGCAGCCCCGTCAAGGCGGGA | |
| | | | GTGGAGACCACCAAACCCTCCAAA | |
| | | | CAGAGCAACAACAAGTACGCGGCC | |
| | | | AGCAGCTA | |
| S166-2620 | GAGGTGCAGCTGGTGGAGTCTGGGG | 2717 | TCCTATGAGCTGACTCAGCCACCCT | 2766 |
| | GAGGCTTGGTCCAGCCTGGGGGGTC | | CAGTGTCCGTGTCCCCAGGACAGA | |
| | CCTGAGACTCTCCTGTGCAGCCTCT | | CAGCCAGCATCACCTGCTCTGGAG | |
| | GGATTCACCTTTAGTAGCTATTGGA | | ATAAATTGGGGGATAAATATGCTT | |
| | TGAGCTGGGTCCGCCAGGCTCCAGG | | GCTGGTATCAGCAGAAGCCAGGCC | |
| | GAAGGGGCTGGAGTGGGTGGCCAA | | AGTCCCCTGTGCTGGTCATCTATCA | |
| | CATAAAGCAAGATGGAAGTGAGAA | | AGATAGCAAGCGGCCCTCAGGGAT | |
| | ATACTATGTGGCCTCTGTGAAGGGC | | CCCTGAGCGATTCTCTGGCTCCAAC | |
| | CGATTCACCATCTCCAGAGACAACG | | TCTGGGAACACAGCCACTCTGACC | |
| | CCAAGAACTCACTGTATCTGCAAAT | | ATCAGCGGGACCCAGGCTATGGAT | |
| | GAACAGCCTGAGAGCCGAGGACAC | | GAGGCTGACTATTTCTGTCAGGCGT | |
| | GGCCGTGTATTACTGTGCGAGAGAT | | GGGACAGCAGCACTGTGGTATTCG | |
| | AGTATAGCAGTGGCTGGGGGCCTTG | | GCGGAGGGACCAAGCTGACCGTCC | |
| | ACTACTGGGGCCAGGGAACCCTGGT | | TACGTCAGCCCAAGGCTGCCCCCT | |
| | CACCGTCTCCTCAGGGAGTGCATCC | | CGGTCACTCTGTTCCCGCCCTCCTC | |
| | GCCCCAACCCTTTTCCCCCTCGTCTC | | TGAGGAGCTTCAAGCCAACAAGGC | |
| | CTGTGAGAATTCCCCGTCGGATACG | | CACACTGGTGTGTCTCATAAGTGA | |
| | AGCAGCGTG | | CTTCTACCCGGGAGCCGTGACAGT | |
| | | | GGCCTGGAAGGCAGATAGCAGCCC | |
| | | | CGTCAAGGCGGGAGTGGAGACCAC | |
| | | | CACACCCTCCAAACAAAGCAACAA | |
| | | | CAAGTACGCGGCCAGCAGCTA | |
| S166-32 | CAGGTGCAGCTGGTGGAGTCTGGGG | 2718 | GACATCCAGATGACCCAGTCTCCTT | 2767 |
| | GAGGCTTGGTCAAGCCTGGAGGGTC | | CCACCCTGTCTGCATCTGTAGGAG | |
| | CCTGAGACTCTCCTGTGCAGCCTCT | | ACAGAGTCACCATCACTTGCCGGG | |
| | GGATTCACCTTCAGTGACTACTACA | | CCAGTCAGAGTATTTTTAGCTGGTT | |
| | TGAGCTGGATCCGCCAGGCTCCAGG | | GGCCTGGTATCAGCAGAAACCAGG | |
| | GAAGGGGCTGGAGTGGGTTTCATAC | | GAAAGCCCCTAAGCTCCTGATCTA | |
| | ATTAGTATTAGTGATACGACCATAT | | TGATGCCTCCAGTTTGGAAAGTGG | |
| | ACTACGCAGACGCTGTGCAGGGCCG | | GGTCCCATCAAGGTTCAGCGGCAG | |
| | ATTCACCATGTCCAGGGACAACGCC | | TGGATCTGGGACAGAATTCACTCT | |
| | AAGAACTCACTGTATCTGCAAATGA | | CACCATCAGCAGCCTGCAGCCTGA | |
| | ACAGCCTGAAGGCCGAGGACACGG | | TGATTTTGCAACTTATTACTGCCAA | |
| | CCGTGTATTACTGTGCGAGAGCTAG | | CAGTATAATAGTTATTGGACGTTCG | |
| | CCCATATTGTGGTGGTGATTGCTCTT | | GCCAAGGGACCAAGGTGGAAATCA | |
| | TCGGCAATGCTTTTGATATCTGGGG | | AACGAACTGTGGCTGCACCATCTG | |
| | CCTAGGGACAATGGTCACCGTCTCT | | TCTTCATCTTCCCGCCATCTGATGA | |
| | TCAGCCTCCACCAAGGGCCCATCGG | | GCAGTTGAAATCTGGAACTGCCTC | |
| | TCTTCCCCCTGGCACCCTCCTCCAAG | | TGTTGTGTGCCTGCTGAATAACTTC | |
| | AGCACCTCTGGGGGCACAGCGGCCC | | TATCCCAGAGAGGCCAAAGTACAG | |
| | TGGGCTGCCTGGTCAAGGACTACTT | | TGGAAGGTGGATAACGCCCTCCAA | |
| | CCCCGAACCGGTGACGGTGTCGTGG | | TCGGGTAACTCCCAGGAGAGTGTC | |
| | AACTCAGGCGCCCTGACCAGCGGCG | | ACAGAGCAGGACAGCAAGGACAG | |
| | TGCACACCTTCCCGGCTGTCCTACA | | CACCTACAGCCTCAGCAGCACCCT | |
| | GTCCTCAGGA | | GACGCTGAGCAAAGCAGACTACGA | |
| | | | G | |
| S171-1150 | GAGGTGCAGCTGGTGGAGTCTGGGG | 2719 | TCCTATGAGCTGACTCAGCCACCCT | 2768 |
| | GAGGCTTGGTCCAGCCTGGGGGGTC | | CAGTGTCCGTGTCCCCAGGACAGA | |
| | CCTGAGACTCTCCTGTGCAGCCTCT | | CAGCCAGCATCACCTGCTCTGGAG | |
| | GGATTCACCTTTAGTAGCTATTGGA | | ATAAATTGGGGGATAAATATGCTT | |
| | TGAGCTGGGTCCGCCAGGCTCCAGG | | GCTGGTATCAGCAGAAGCCAGGCC | |
| | GAAGGGGCTGGAGTGGGTGGCCAA | | AGTCCCCTGTGCTGGTCATCTATCA | |
| | CATAAAGCAAGATGGAAGTGAGAA | | AGATAGCAAGCGGCCCTCAGGGAT | |
| | ATACTATGTGGACTCTGTGAAGGGC | | CCCTGAGCGATTCTCTGGCTCCAAC | |
| | CGATTCACCATCTCCAGAGACAACG | | TCTGGGAACACAGCCACTCTGACC | |
| | CCAAGAACTCACTGTATCTGCAAAT | | ATCAGCGGGACCCAGGCTATGGAT | |
| | GAACAGCCTGAGAGCCGAGGACAC | | GAGGCTGACTATTACTGTCAGGCG | |
| | GGCTGTGTATTACTGTGCGAGAGAC | | TGGGACAGCAGCACTGTGGTATTC | |
| | GGTATAGCAGTGGCTGGTGGGCTTG | | GGCGGAGGGACCAAGCTGACCGTC | |
| | ACTACTGGGGCCAGGGAACCCTGGT | | CTAGGTCAGCCCAAGGCTGCCCCC | |
| | CACCGTCTCCTCAGCACCCACCAAG | | TCGGTCACTCTGTTCCCGCCCTCCT | |
| | GCTCCGGATGTGTTCCCCATCATAT | | CTGAGGAGCTTCAAGCCAACAAGG | |
| | CAGGGTGCAGACACCCAAAGGATA | | CCACACTGGTGTGTCTCATAAGTG | |
| | ACAGCCCTGTGGTCCTGGCATGCTT | | ACTTCTACCCGGGAGCCGTGACAG | |
| | GATAACTGGGTACCACC | | TGGCCTGGAAGGCAGATAGCAGCC | |
| | | | CCGTCAAGGCGGGAGTGGAGACCA | |
| | | | CCACACCCTCCAAACAAAGCAACA | |
| | | | ACAAGTACGCGGCCAGCAGCTA | |
| S171-1285 | CAGGTGCAGTTGGTGGAGTCTGGGG | 2720 | TCCTATGAGCTGACACAGCCACCC | 2769 |
| | GAGGCGTGGTCCAGCCTGGGAGGTC | | TCGGTGTCAGTGTCCCCAGGACAA | |
| | CCTGAGACTCTCCTGTGCAGCCTCT | | ACGGCCAGGATCACCTGCTCTGGA | |
| | GGATTCATCTTCAGTAACAATGCTT | | GATGCACTGCCAAAAAAATTTGTT | |
| | TGCACTGGGTCCGCCAGGCTCCAGG | | CATTGGTACCAGCAGAAGTCAGGC | |
| | CAAGGGGCTGGAGTGGGTGGCAATT | | CAGGCCCCTGTGCTGGTCATCTATG | |
| | ATATCATATGATGGAAGCAATAAAA | | AGGACAGTAAACGACCCTCCGGGA | |
| | ATTATGCAGCCTCCGTGAAGGGCCG | | TCCCTGAGAGATTCTCTGGCTCCAG | |
| | ATTCACCATCTCCAGAGACAATTCC | | CTCAGGGACAACGGCCACCTTGAC | |
| | CAGAACACGGTGTTTCTGCAAATGA | | CATCAGTGGGGCCCAGGTGGAGGA | |
| | ACAGCCTGAGAGCTGAAGACACGG | | TGAAGGTGACTACTACTGTTATTCA | |
| | CTGTGTATTACTGTGCGAGAGATCA | | ACAGACAGTAGTGGCCGAGGGGTG | |
| | TATAGCAGGAGCTGCTAAGTATTTC | | TTCGGCGGAGGGACCAAGCTGACC | |
| | GACTACTGGGGCCAGGGAACCCTGG | | GTCCTAGGTCAGCCCAAGGCTGCC | |
| | TCACCGTCTCCTCAGCCTCCACCAA | | CCCTCGGTCACTCTGTTCCCACCCT | |
| | GGGCCCATCGGTCTTCCCCCTGGCA | | CCTCTGAGGAGCTTCAAGCCAACA | |
| | CCCTCCTCCAAGAGCACCTCTGGGG | | AGGCCACACTGGTGTGTCTCATAA | |
| | GCACAGCGGCCCTGGGCTGCCTGGT | | GTGACTTCTACCCGGGAGCCGTGA | |
| | CAAGGACTACTTCCCCGAACCGGTG | | CAGTGGCCTGGAAGGCAGATAGCA | |
| | ACGGTGTCGTGGAACTCAGGCGCCC | | GCCCCGTCAAGGCGGGAGTGGAGA | |
| | TGACCAGCGGCGTGCACACCTTCCC | | CCACCACACCCTCCAAACAAAGCA | |
| | GGCTGTCCTACAGTCCTCAGGA | | ACAACAAGTACGCGGCCAGCAGCT | |
| | | | A | |
| S171-692 | CAGGTGCAGCTGCAGGAGTCGGGCC | 2721 | GACATCCAGATGACCCAGTCTCCA | 2770 |
| | CAGGACTGGTGAAGCCTTCACAGAC | | TCCTCCCTGTCTGCATCTGTAGGAG | |
| | CCTGTCCCTCACCTGCACTGTCTCTG | | ACAGAGTCACCATCACTTGCCGGG | |
| | GTGGCTCCATCAGCAGTGGTAGTTA | | CAAGTCAGAGCATTAGCAGCTATT | |
| | CTACTGGAGCTGGATCCGGCAGCCC | | TAAATTGGTATCAGCAGAAACCAG | |
| | GCCGGGAAGGGACTGGAGTGGATT | | GGAAAGCCCCTAAGCTCCTGATCT | |
| | GGGCGTATCTATACCAGTGGGAGCA | | ATGCTGCATCCAGTTTGCAAAGTG | |
| | CCAACTACAACCCCTCCCTCAAGAG | | GGGTCCCATCAAGGTTCAGTGGCA | |
| | TCGAGTCACCATATCAGTAGACACG | | GTGGATCTGGGACAGATTTCACTCT | |
| | TCCAAGAACCAGTTCTCCCTGAAGC | | CACCATCAGCAGTCTGCAACCTGA | |
| | TGAGCTCTGTGACCGCCGCAGACAC | | AGATTTTGCAACTTACTACTGTCAA | |
| | GGCCGTGTATTACTGTGCGAGAGAG | | CAGAGTTACAGTAAGAACACTTTT | |
| | AGTAAGGTAACTATGGTTCGGGGAG | | GGCCAGGGGACCAAGCTGGAGATC | |
| | GTCTGGCCTACTACTACATGGACGT | | AAACGAACTGTGGCTGCACCATCT | |
| | CTGGGGCAAAGGGACCACGGTCAC | | GTCTTCATCTTCCCGCCATCTGATG | |
| | CGTCTCCTCAGCACCCACCAAGGCT | | AGCAGTTGAAATCTGGAACTGCCT | |
| | CCGGATGTGTTCCCCATCATATCAG | | CTGTTGTGTGCCTGCTGAATAACTT | |
| | GGTGCAGACACCCAAAGGATAACA | | CTATCCCAGAGAGGCCAAAGTACA | |
| | GCCCTGTGGTCCTGGCATGCTTGAT | | GTGGAAGGTGGATAACGC | |
| | AACTGGGTACCACC | | | |
| S179-122 | GAGGTGCAGCTGGTGGAGTCTGGGG | 2722 | AATTTTATGCTGACTCAGCCCCACT | 2771 |
| | GAGGCTTGGTCCAGCCTGGGGGGTC | | CTGTGTCGGAGTCTCCGGGGAAGA | |
| | CCTGAGACTCTCCTGTGCAGCCTCT | | CGGTAACCATCTCCTGCACCGGCA | |
| | GGATTCACCTTTAGTACCTATTGGA | | GCAGTGGCAGCATTGCCAGCAACT | |
| | TGAGCTGGGTCCGCCAGGCTCCAGG | | ATGTGCAGTGGTACCAGCAGCGCC | |
| | GAAGGGGCTGGAGTGGGTGGCCAA | | CGGGCAGTGCCCCCACCACTGTGA | |
| | CATAAAGCAAGATGGAAGTGAGAA | | TCTATGAGGATAACCAAAGACCCT | |
| | GTACTATGTGGACTCTGTGAAGGGC | | CTGGGGTCCCTGATCGGTTCTCTGG | |
| | CGATTCACCATCTCCAGAGACAACG | | CTCCATCGACAGCTCCTCCAACTCT | |
| | CCAAGAACTCACTGTATCTGCAAAT | | GCCTCCCTCACCATCTCTGGACTGA | |
| | GAACAGCCTGAGAGCCGAGGACAC | | AGACTGAGGACGAGGCTGACTACT | |
| | GGCCGTGTATTACTGTGCGTCTAAG | | ACTGTCAGTCTTATGATAGCAGCA | |
| | CTATGGTTACGTGGAAACTTTGACT | | ATCTAGTGTTCGGCGGAGGGACCA | |
| | ACTGGGGCCAGGGAACCCTGGTCAC | | AGCTGACCGTCCTAGGTCAGCCCA | |
| | CGTCTCCTCAGCCTCCACCAAGGGC | | AGGCTGCCCCCTCGGTCACTCTGTT | |
| | CCATCGGTCTTCCCCCTGGCACCCTC | | CCCGCCCTCCTCTGAGGAGCTTCAA | |
| | CTCCAAGAGCACCTCTGGGGGCACA | | GCCAACAAGGCCACACTGGTGTGT | |
| | GCGGCCCTGGGCTGCCTGGTCAAGG | | CTCATAAGTGACTTCTACCCGGGA | |
| | ACTACTTCCCCGAACCGGTGACGGT | | GCCGTGACAGTGGCCTGGAAGGCA | |
| | GTCGTGGAACTCAGGCGCCCTGACC | | GATAGCAGCCCCGTCAAGGCGGGA | |
| | AGCGGCGTGCACACCTTCCCGGCTG | | GTGGAGACCACCACACCCTCCAAA | |
| | TCCTACAGTCCTCAGGA | | CAAAGCAACAACAAGTACGCGGCC | |
| | | | AGCAGCTACCTGAGCCTGACGCCT | |
| | | | GAGCAGTGGAAGTCCCAC | |
| S179-20 | CAGGTGCAGCTGGTGGAGTCTGGGG | 2723 | GAAGTAGTGCTGACGCAGTCTCCA | 2772 |
| | GAGGCGTGGTCCAGCCTGGGAGGTC | | GCCACCCTGTCTGTGTCTCCAGGGG | |
| | CCTGAGACTCTCCTGTGCAGCGTCT | | AAAGAGCCACCCTCTCCTGCAGGG | |
| | GGATTCACCTTCAGTGGCTATGGCA | | CCAGTCAGAGTGTTAGCAGCAATT | |
| | TGCACTGGGTCCGCCAGGCTCCAGG | | TAGCCTGGTATCAGCAGAAACCTG | |
| | CAAGGGGCTGGAGTGGGTGGCAGTT | | GCCAGGCTCCCAGGCTCCTCATCTA | |
| | ATATGGTTTGATGGAAGTAATAAAT | | TGGTGCATCCACCAGGGCCACTGG | |
| | ACTATGCAGACTCCGTGAAGGGCCG | | TATCCCAGCCAGGTTCAGTGGCAG | |
| | ATTCACCATCTCCAGAGACAATTCC | | TGGGTCTGGGACAGAGTTCACTCT | |
| | AAGAACACGCTGTATCTGCAAATGA | | CACCATCAGCAGCCTGCAGTCTGA | |
| | ACAGCCTGAGAGCCGAGGACACGG | | AGATTTTGCAGTTTATTACTGTCAG | |
| | CTGTCTATTACTGTGCGAGAGATGC | | CAGTATAATAACTGGCCTCGGACG | |
| | GCGTTACTATGATACTAGTGGTTAT | | TTCGGCCAAGGGACCAAGGTGGAA | |
| | TTAGGGACAACAGAGTTTGACTACT | | ATCAAACGAACTGTGGCTGCACCA | |
| | GGGGCCAGGGAACCCTGGTCACCGT | | TCTGTCTTCATCTTCCCGCCATCTG | |
| | CTCCTCAGGGAGTGCATCCGCCCCA | | ATGAGCAGTTGAAATCTGGAACTG | |
| | ACCCTTTTCCCCCTCGTCTCCTGTGA | | CCTCTGTTGTGTGCCTGCTGAATAA | |
| | GAATTCCCCGTCGGATACGAGCAGC | | CTTCTATCCCAGAGAGGCCAAAGT | |
| | GTGGCC | | ACAGTGGAAGGTGGATAACGCCCT | |
| | | | CCAATCGGGTAACTCCCAGGAGAG | |
| | | | TGTCACAGAGCAGGACAGCAAGGA | |
| | | | CAGCACCTACAGCCTCAGCAGCAC | |
| | | | CCTGACGCTGAGCAAAGCAGACTA | |
| | | | CGAGAA | |
| S179-27 | CAGGTGCAGCTGGTGGAGTCTGGGG | 2724 | GACATCCAGATGACCCAGTCTCCA | 2773 |
| | GAGGCGTGGTCCAGCCTGGGAGGTC | | TCCTCCCTGTCTGCATCTGTAGGAG | |
| | CCTGAGACTCTCCTGTGCAGCCTCT | | ACAGAGTCACCATCACTTGCCAGG | |
| | GGATTCACCTTCAGGAGCTATGGCA | | CGAGTCAGGACATTAGCAACTATT | |
| | TGCACTGGGTCCGCCAGGCTCCAGG | | TAAATTGGTATCAGCAGAAACCAG | |
| | CAAGGGGCTGGAGTGGGTGGCAGTT | | GGAAAGCCCCTAAGCTCCTGATCT | |
| | ATATCATATGATGGAAGTAATAAAA | | ACGATGCATCCAATTTGGAAACAG | |
| | ACTATGCAGACTCCGTGAAGGGCCG | | GGGTCCCATCAAGGTTCAGTGGAA | |
| | ACTCACCATCTCCAGAGACAATTCC | | GTGGATCTGGGACAGATTTTACTTT | |
| | AAGAACACGCTGTATCTGCAAATGA | | CACCATCAGCAGCCTGCAGCCTGA | |
| | ACAGCCTGAGAGCTGAGGACACGG | | AGATATTGCAACATATTACTGTCA | |
| | CTGTGTATTACTGTGCGAAAGATCG | | ACAATATGATAATCTCCCCCTCACT | |
| | GGGTGGGTATAGCAGTGGCTGGACC | | TTCGGCGGAGGGACCAAGGTGGAG | |
| | TACTACTACTACGGTATGGACGTCT | | ATCAAACGAACTGTGGCTGCACCA | |
| | GGGGCCAAGGGACCACGGTCACCG | | TCTGTCTTCATCTTCCCGCCATCTG | |
| | TCTCCTCAGCCTCCACCAAGGGCCC | | ATGAGCAGTTGAAATCTGGAACTG | |
| | ATCGGTCTTCCCCCTGGCACCCTCCT | | CCTCTGTTGTGTGCCTGCTGAATAA | |
| | CCAAGAGCACCTCTGGGGGCACAGC | | CTTCTATCCCAGAGAGGCCAAAGT | |
| | GGCCCTGGGCTGCCTGGTCAAGGAC | | ACAGTGGAAGGTGGATAACGCCCT | |
| | TACTTCCCCGAACCGGTGACGGTGT | | CCAATCGGGTAACTCCCAGGAGAG | |
| | CGTGGAACTCAGGCGCCCTGACCAG | | TGTCACAGAGCAGGACAGCAAGGA | |
| | CGGCGTGCACACCTTCCCGGCTGTC | | CAGCACCTACAGCCTCAGCAGCAC | |
| | CTACAGTCCTCAGGACTCTACTCCC | | CCTGACGCTGAGCAAAGCAGACTA | |
| | TCAGCAGCGTGGTGACCGTGCCCTC | | CGAGAA | |
| | CAGCAGCTTGGGCACCCAGACCTAC | | | |
| | ATCTGCAACGTGAATCACAAGCCCA | | | |
| | GCAACACCAAGGTGGACA | | | |
| S179-28 | GAGGTGCAGCTGTTGGAGTCTGGGG | 2725 | GACATCCAGATGACCCAGTCTCCTT | 2774 |
| | GAGGCTTGGTACAGCCTGGGGGGTC | | CCACCCTGTCTGCATCTGTAGGAG | |
| | CCTGAGACTCTCCTGTGCAGCCTCT | | ACAGAGTCACCATCACTTGCCGGG | |
| | GGATTCACCTTTAGCAGCTATGCCA | | CCAGTCAGAGTATTACTAGCTGGTT | |
| | TGAGCTGGGTCCGCCAGGCTCCAGG | | GGCCTGGTATCAGCAGAAACCAGG | |
| | GAAGGGGCTGGAGTGGGTCTCAGCT | | GAAAGCCCCTAAGCTCCTGATCTA | |
| | ATTAGGGGTAGTGGTGGTAGCACAT | | TGATGCCTCCAGTTTGGAAAGTGG | |
| | ACTACGCAGACTCCGTGAAGGGCCG | | GGTCCCATCAAGGTTCAGCGGCAG | |
| | GTTCACCATCTCCAGAGACAATTCC | | TGGATCTGGGACAGAATTCACTCT | |
| | AAGAACACACTGTATCTGCAAATGA | | CACCATCAGCAGCCTGCAGCCTGA | |
| | ACAGCCTGAGAGCCGAGGACACGG | | TGATTTTGCAACTTATTACTGCCAA | |
| | CCGTATATTACTGTGCGAAAGGGGT | | CATTATAATAGTTATCCTTGGACGT | |
| | CCGCAGCTCGGATGACTACTTTGAG | | TCGGCCAAGGGACCAAGGTGGAAA | |
| | TACTGGGGCCAGGGAACCCTGGTCA | | TCAAACGAACTGTGGCTGCACCAT | |
| | CCGTCTCCTCAGCCTCCACCAAGGG | | CTGTCTTCATCTTCCCGCCATCTGA | |
| | CCCATCGGTCTTCCCCCTGGCACCCT | | TGAGCAGTTGAAATCTGGAACTGC | |
| | CCTCCAAGAGCACCTCTGGGGGCAC | | CTCTGTTGTGTGCCTGCTGAATAAC | |
| | AGCGGCCCTGGGCTGCCTGGTCAAG | | TTCTATCCCAGAGAGGCCAAAGTA | |
| | GACTACTTCCCCGAACCGGTGACGG | | CAGTGGAAGGTGGATAACGCCCTC | |
| | TGTCGTGGAACTCAGGCGCCCTGAC | | CAATCGGGTAACTCCCAGGAGAGT | |
| | CAGCGGCGTGCACACCTTCCCGGCT | | GTCACAGAGCAGGACAGCAAGGAC | |
| | GTCCTACAGTCCTCAGGACTCTACT | | AGCACCTACAGCCTCAGCAGCACC | |
| | CCCTCAGCAGCGTGGTGACCGTGCC | | CTGACGCTGAGCAAAGCAGACTAC | |
| | CTCCAGCAGCTTGGGCACCCAGACC | | GAGAA | |
| | TACATCTGCAACGTGAATCACAAGC | | | |
| | CCAGCAACACCAAGGTGGACA | | | |
| S210-1139 | GAGGTGCAGCTGGTGCAGTCTGGAG | 2726 | GAAATTGTGTTGACGCAGTCTCCA | 2775 |
| | CAGAGGTGAAAAAGCCCGGAGAGT | | GGCACCCTGTCTTTGTCTCCAGGGG | |
| | CTCTGAAGATCTCCTGTAAGGGTTC | | AAAGAGCCACCCTCTCCTGCAGGG | |
| | TGGATACTACTTTCCCAGCTACTGG | | CCAGTCAGAGTGTTAGCAGCAGCT | |
| | ATCGGCTGGGTGCGCCAGAAGCCCG | | ACTTAGCCTGGTACCAGCAGAAAC | |
| | GGAATGGCCCGGAGTGGATGGGAA | | CTGGCCAGGCTCCCAGACTCCTCAT | |
| | TCATCCATCCTGGTGACTCTGAAAG | | CTATGGTGCATCTAGCAGGGCCAC | |
| | CACATACAGCCCGTCCTTCCAAGGC | | TGGCATCCCAGACAGGTTCAGTGG | |
| | CAGGTCACCATCTCGGCCGACAAGT | | CAGTGGGTCTGGGACAGACTTCAC | |
| | CCATCAGCACCGCCTACCTGCAGTG | | TCTCACCATCAGCAGACTGGAGGC | |
| | GAGCAGCCTGAAGGCCTCGGACACC | | TGAAGATTTTGCAGTATATTACTGT | |
| | GCCATGTATTACTGTGCGCGACCGT | | CAGCTCTTTGGTAGCTCACCGACGT | |
| | TTTACTATGGTTCGGAGAGTCCCCC | | GGACGTTCGGCCAAGGGACCAAGG | |
| | CGGCTACTGGGGCCAGGGAACCCTG | | TGGAAATCAAACGAACTGTGGCTG | |
| | GTCACCGTCTCCTCAGGGAGTGCAT | | CACCATCTGTCTTCATCTTCCCGCC | |
| | CCGCCCCAACCCTTTTCCCCCTCGTC | | ATCTGATGAGCAGTTGAAATCTGG | |
| | TCCTGTGAGAATTCCCCGTCGGATA | | AACTGCCTCTGTTGTGTGCCTGCTG | |
| | CGAGCAGCGTG | | AATAACTTCTATCCCAGAGAGGCC | |
| | | | AAAGTACAGTGGAAGGTGGATAAC | |
| | | | GC | |
| S210-1262 | CAGCTGCAGCTGCAGGAGTCGGGCC | 2727 | CAGCTTGTGCTGACTCAATCGCCCT | 2776 |
| | CAGGACTAATGAAGCCTTCGGAGAC | | CTGCCTCTGCCTCCCTGGGAGCCTC | |
| | CCTGTCCCTCACCTGCACTGTCTCTG | | GGTCAAGCTCACCTGCACTCTGAG | |
| | GTGGCTCCATCAGCAGAAGCAATTA | | CAGTGGGCACAGCAGCTACGCCAT | |
| | CTACTGGGGCTGGATCCGCCAGCCC | | CGCATGGCATCAGCAGCAGCCAGA | |
| | CCAGGTAAGGGACTGGAGTGGATTG | | GAGGGGCCCTCGGTACTTGATGAA | |
| | GGAGTATCTATTATAGTGGGAGCAC | | GCTTAACGGTGATGGCAGCCACAG | |
| | CTACTACAACCCCTCCCTCAAGAGT | | CAAGGGGGACGGGATCCCTGATCG | |
| | CGAGTCACCATATCCGTAGACACGT | | CTTCTCAGGCTCCAGCTCTGGGGCT | |
| | CCCAGAACCAGTTCTCCCTGAAGAT | | GAGCGCTACCTCACCATCTCCAGC | |
| | GAGCTCTGTGACCGCCGCAGACACG | | CTCCAGTCTGAAGATGAGGCTGAC | |
| | GCTGTTTATTACTGTGCGAGCCTCTT | | TATTACTGTCAGACCTGGGGCACT | |
| | CGACTACGGTGACAACTACTGGGGC | | GACATTCAAGTGTTCGGCGGAGGG | |
| | CAGGGAACCCTGGTCACCGTCTCCT | | ACCAAGCTGACCGTCCTAGGTCAG | |
| | CAGCCTCCACCAAGGGCCCATCGGT | | CCCAAGGCTGCCCCCTCGGTCACTC | |
| | CTTCCCCCTGGCACCCTCCTCCAAG | | TGTTCCCGCCCTCCTCTGAGGAGCT | |
| | AGCAC | | TCAAGCCAACAAGGCCACACTGGT | |
| | | | GTGTCTCATAAGTGACTTCTACCCG | |
| | | | GGAGCCGTGACAGTGGCCTGGAAG | |
| | | | GCAGATAGCAGCCCCGTCAAGGCG | |
| | | | GGAGTGGAGACCACCACACCCTCC | |
| | | | AAACAAAGCAACAACAAGTACGCG | |
| | | | GCCAGCAGCTA | |
| S210-1611 | CAGGTGCAGCTGGTGCAGTCTGGGG | 2728 | GAAATTGTGTTGACACAGTCTCCA | 2777 |
| | CTGAGGTGAAGAAGCCTGGGTCCTC | | GCCACCCTGTCTTTGTCTCCAGGGG | |
| | GGTGAAGGTCTCCTGCAAGGCTTCT | | AAAGAGCCACCCTCTCCTGCAGGG | |
| | GGAGGCACCTTCAGCAGCTATGCTA | | CCAGTCAGAGTATTAGCAGCTTCTT | |
| | TCAGCTGGGTGCGACAGGCCCGTGG | | AGCCTGGTACCAACAGAAACCTGG | |
| | ACAAGGGCTTGAGTGGATGGGAGG | | CCAGGCTCCCAGGCTCCTCATCTAT | |
| | GATCATCCCTATCTTTGGTACAGCA | | GATGCATCCAACAGGGCCACTGGC | |
| | AACTACCCACAGAAGTTCCAGGGCA | | ATCCCAGCCAGGTTCAGTGGCAGT | |
| | GAGTCACGATTACCGCGGACGAATC | | GGGTCTGGGACAGACTTCATTCTC | |
| | CACGAGCACAGCCTACATGGAGCTG | | ACCATCAACAACCTAGAGCCTGAA | |
| | AGCAGCCTGAGATCTGAGGACACG | | GATTTTGCAGTTTATTACTGTCAGC | |
| | GCCGTGTATTACTGTGCGAGATATC | | AGCGTAGCAACTGGCCTCCGAAGC | |
| | ACGCCTATGATAGTAGTGGCTATTA | | TCACTTTCGGCGGAGGGACCAAGG | |
| | CGTTGACTATTGGGGCCAGGGAACC | | TGGAGATCAAACGAACTGTGGCTG | |
| | CTGGTCACCGTCTCCTCAGCATCCC | | CACCATCTGTCTTCATCTTCCCGCC | |
| | CGACCAGCCCCAAGGTCTTCCCGCT | | ATCTGATGAGCAGTTGAAATCTGG | |
| | GAGCCTCTGCAGCACCCAGCCAGAT | | AACTGCCTCTGTTGTGTGCCTGCTG | |
| | GGGAACGTGGTCATCGCCTGCCTGG | | AATAACTTCTATCCCAGAGAGGCC | |
| | TCCAGGGCTTCTTCCCCCAGGAGCC | | AAAGTACAGTGGAAGGTGGATAAC | |
| | ACTCAGTGTGACCTGGAGCGAAAGC | | GC | |
| | GGACAGGGCGTGACCGCCAGAAAC | | | |
| | TTCCC | | | |
| S210-727 | CAGGTGCAGCTGCAGGAGTCGGGCC | 2729 | GACATCCAGATGACCCAGTCTCCA | 2778 |
| | CAGGACTGGTGAAGCCTTCGGAGAC | | TCCTCACTGTCTGCATCTGTAGGAG | |
| | CCTGTCCCTCACCTGCACTGTCTCTG | | ACAGAGTCACCATCACTTGTCGGG | |
| | GTGGCTCCATGAGTAGCAGTTACTG | | CGAGTCAGGGCATTAGCAGTTATT | |
| | GAGCTGGATCCGGCAGCCCCCAGGG | | TAGCCTGGTTTCAGCAGAAACCAG | |
| | AAGGGACTGGAGTGGATTGGCTATA | | GGAAAGCCCCTAAGTCCCTGATCT | |
| | TCTATTACAGAGGGAGCACCAACTA | | ATGCTGCATCCAGTTTGCAAAGTG | |
| | CAACCCCTCCCTCAAGACTCGAGTC | | GGGTCCCATCAAAGTTCAGCGGCA | |
| | ACCATGTCAGTAGACACGTCCAAGA | | GTGGATCTGGGACAGATTTCACTCT | |
| | ACCAATTCTCGATGAAAATGACCTT | | CACCATCAGCAGCCTGCAGCCTGA | |
| | TATGACCGCTGCGGACACGGCCGTC | | AGATTTTGCAACTTATTACTGCCAA | |
| | TATTACTGTGCGCGAGAGGCGGCGT | | CAGTATAATAGATACCCTCCCACTT | |
| | TCAACTGGTTCGACTCCTGGGGCCA | | TCGGCGGAGGGACCAAGGTGGAGA | |
| | GGGAACCCTGGTCACCGTCTCCTCA | | TCAAGCGAACTGTGGCTGCACCAT | |
| | GGGAGTGCATCCGCCCCAACCCTTT | | CTGTCTTCATCTTCCCGCCATCTGA | |
| | TCCCCCTCGTCTCCTGTGAGAATTCC | | TGAGCAGTTGAAATCTGGAACTGC | |
| | CCGTCGGATACGAGCAGCGTG | | CTCTGTTGTGTGCCTGCTGAATAAC | |
| | | | TTCTATCCCAGAGAGGCCAAAGTA | |
| | | | CAGTGGAAGGTGGATAACGC | |
| S210-852 | GAGGTGCAGCTGGTGGAGTCTGGGG | 2730 | TCCTATGAGCTGACTCAGCCACCCT | 2779 |
| | GAGGCTTGGTCCAGCCTGGGGGGTC | | CAGTGTCCGTGTCCCCAGGACAGA | |
| | CCTGAGACTCTCCTGTGCAGCCTCT | | CAGCCAGCATCACCTGCTCTGGAG | |
| | GGATTCACCTTAAGTATTTATTGGA | | ATAAATTGGGGGATACATATGCTT | |
| | TGAGCTGGGTCCGCCAGGCTCCAGG | | GCTGGTATCAGCAGAAGCCAGGCC | |
| | GAAGGGGCTGGAGTGGGTGGCCAA | | AGTCCCCTGTACTGGTCATCTATCA | |
| | CATAAAGCAAGATGGACGTGAGAA | | AGATAGCAAGCGGCCCTCAGGGAT | |
| | ATACCATGTGGACTCTGTGAAGGGC | | CCCTGAGCGATTCTCTGGCTCCAAC | |
| | CGATTCACCATCTCCAGAGACAACG | | TCTGGGAACACAGCCACTCTGACC | |
| | CCAACAACTCACTGTATCTGCAAAT | | ATCAGCGGGACCCAGGCTATGGAT | |
| | GAACAACCTGAGAGCCGAGGACAC | | GAGGCTGACTATTACTGTCAGGCG | |
| | GGCTGTGTATTTCTGTGCGAGAGAT | | TGGGACAGCAGCACGTCTGTGGTA | |
| | GGTATAGCAGTGGCTGGGGGGTTTG | | TTCGGCGGAGGGACCAAGCTGACC | |
| | ACTACTGGGGCCAGGGAACCCTGGT | | GTCCTAGGTCAGCCCAAGGCTGCC | |
| | CACCGTCTCCTCAGGGAGTGCATCC | | CCCTCGGTCACTCTGTTCCCGCCCT | |
| | GCCCCAACCCTTTTCCCCCTCGTCTC | | CCTCTGAGGAGCTTCAAGCCAACA | |
| | CTGTGAGAATTCCCCGTCGGATACG | | AGGCCACACTGGTGTGTCTCATAA | |
| | AGCAGCGTG | | GTGACTTCTACCCGGGAGCCGTGA | |
| | | | CAGTGGCCTGGAAGGCAGATAGCA | |
| | | | GCCCCGTCAAGGCGGGAGTGGAGA | |
| | | | CCACCACACCCTCCAAACAAAGCA | |
| | | | ACAACAAGTACGCGGCCAGCAGCT | |
| | | | A | |
| S210-896 | CAGGTGCAGCTGGTGGAGTCTGGGG | 2731 | GAAATTGTGTTGACGCAGTCTCCA | 2780 |
| | GAGGCGTGGTCCAGCCTGGGAGGTC | | GGCACCCTGTCTTTGTCTCCAGGGG | |
| | CCTGAGACTCTCCTGTGCAGCCTCT | | AAAGAGCCACCCTCTCCTGCAGGG | |
| | GGATTCACCTTCAGTAGCTATGCTA | | CCAGTCAGAGTATTAGCAGCAACT | |
| | TGCACTGGGTCCGCCAGGCTCCAGG | | ACTTAGCCTGGTACCAGCAGAAAC | |
| | CAAGGGGCTGGAGTGGGTGGCAGTT | | CTGGCCAGGCTCCCAGGCTCCTCAT | |
| | ATATCATATGATGGAGGCAATAAAT | | CTATGGTGCATCCAGCAGGGCCAC | |
| | ACTACGCAGACTCCGTGAAGGGCCG | | TGGCATCCCAGACAGGTTCAGTGG | |
| | ATTCACCATCTCCAGAGACAATTCC | | CAGTGGGTCTGGGACAGACTTCAC | |
| | AAGAACACGCTGTATCTGCAAATGA | | TCTCACCATCAGCAGACTGGAGCC | |
| | ACAGCCTGAGAGCTGAGGACACGG | | TGAAGATTTTGCAGTGTATTACTGT | |
| | CTGTGTATTACTGTGCGAGAGGACA | | CAGCAGTATGGTAGCTCACCTCTC | |
| | TGGGAACTACCTTACCTACTTTGAC | | ACTTTCGGCCCTGGGACCAAAGTG | |
| | TACTGGGGCCAGGGAACCCTGGTCA | | GATATCAAACGAACTGTGGCTGCA | |
| | CCGTCTCCTCAGGGAGTGCATCCGC | | CCATCTGTCTTCATCTTCCCGCCAT | |
| | CCCAACCCTTTTCCCCCTCGTCTCCT | | CTGATGAGCAGTTGAAATCTGGAA | |
| | GTGAGAATTCCCCGTCGGATACGAG | | CTGCCTCTGTTGTGTGCCTGCTGAA | |
| | CAGCGTG | | TAACTTCTATCCCAGAGAGGCCAA | |
| | | | AGTACAGTGGAAGGTGGATAACGC | |
| S2141- | GAGGTGCAGCTGGTGGAGTCCGGG | 2732 | AATTTTATGCTGACTCAGCCCCACT | 2781 |
| 113 | GGAGGCTTAGTTCAGCCTGGGGGGT | | CTGTGTCGGAGTCTCCGGGGAAGA | |
| | CCCTGAGACTCTCCTGTGCAGCCTC | | CGGTAACCATCTCCTGCACCGGCA | |
| | TGGATTCACCTTCAGTAGCTCCTGG | | GCAGTGGCAGCATTGCCAGCAACT | |
| | ATACACTGGGTCCGCCAAGCTCCAG | | ATGTGCAGTGGTACCAGCAGCGCC | |
| | GGAAGGGGCTGGTGTGGGTCTCACG | | CGGGCAGTGCCCCCACCACTGTGA | |
| | TATTAATAGTGATGGGAGTAGCACA | | TCTATGAGGATAACCAAAGACCCT | |
| | ACCTACGCGGACTCCGTGAAGGGCC | | CTGGGGTCCCTGATCGGTTCTCTGG | |
| | GATTCACCATCTCCAGAGACAACGC | | CTCCATCGACAGCTCCTCCAACTCT | |
| | CAAGAACACGCTGTTTCTGCAAATG | | GCCTCCCTCACCATCTCTGGACTGA | |
| | AACAGTCTGAGAGCCGAGGACACG | | AGACTGAGGACGAGGCTGACTACT | |
| | GCTGTGTATTACTGTGCAAGAGCGG | | ACTGTCAGTCTTATGATACCAGCA | |
| | AGTGGCTACGCGGGCAGTTTGACTA | | ATCATGTGGTATTCGGCGGAGGGA | |
| | CTGGGGCCAGGGAACCCTGGTCACC | | CCAAGCTGACCGTCCTAGGTCAGC | |
| | GTCTCCTCACCACCCACCAAGGCTC | | CCAAGGCTGCCCCCTCGGTCACTCT | |
| | CGGATGTGTTCCCCATCATATCAGG | | GTTCCCGCCCTCCTCTGAGGAGCTT | |
| | GTGCAGACACCCAAAGGATAACAG | | CAAGCCAACAAGGCCACACTGGTG | |
| | CCCTGTGGTCCTGGCATGCTTGATA | | TGTCTCATAAGTGACTTCTACCCGG | |
| | ACTGGGTACCACCCAACGTCCGTGA | | GAGCCGTGACAGTGGCCTGGAAGG | |
| | CTGTCACCTGGTACATGGGGACACA | | CAGATAGCAGCCCCGTCAAGGCGG | |
| | GAGCCAGCCCCAGAGAACCTTCCCT | | GAGTGGAGACCACCACACCCTCCA | |
| | GAGATACAAAGACGGGACAGCTAC | | AACAAAGCAACAACAAGTACGCGG | |
| | TACATGACAAGCAGCCAGCTCTCCA | | CCAGCAGCTACCTGAGCCTGACGC | |
| | CCCCCCTCCAGCAGTGGCGCCAAGG | | CTGAGCAGTGGAAGTCCCACA | |
| | CGAGTACAAATGCGTGGTCCAGCA | | | |
| S2141-126 | GAGGTGCAGCTGGTGCAGTCTGGAG | 2733 | GACATCCAGATGACCCAGTCTCCTT | 2782 |
| | CAGAGGTGAAAAACCCGGGGGAGT | | CCACCCTGTCTGCATCTGTAGGAG | |
| | CTCTGAAGATCTCCTGTAAGGGTTC | | ACAGAGTCACCATCACTTGCCGGG | |
| | TGGATACAGGTTTACCACCTACTGG | | CCAGTCAGAGTATTAGTAGCTGGT | |
| | ATCGGCTGGGTGCGCCAGATGCCCG | | TGGCCTGGTATCAGCAGAAACCAG | |
| | GGAAAGGCCTGGAGTGGATGGGGA | | GGAAAGCCCCTAAGCTCCTGATCT | |
| | TCATCTATCCTGGTGACTCTGATACC | | ATGATGCCTCCAGTTTGGAAAGTG | |
| | AGATACAGCCCGTCCTTCGAAGGCC | | GGGTCCCATCAAGGTTCAGCGGCA | |
| | AGGTCACCATCTCAGCCGACAAGTC | | GTGGATCTGGGACAGAATTCACTC | |
| | CATCAGCACCGCCTACCTGCAGTGG | | TCACCATCAGCAGCCTGCAGCCTG | |
| | AGCAGCCTGAAGGCCTCGGACACCG | | ATGACTTTGCAACTTATTACTGCCA | |
| | CCATGTATTACTGTGCGAGGCACCC | | ACAGTATAATAGTCATTGGACGTT | |
| | CCTGGGCTTGGGGGGAAGTATTGAC | | CGGCCAAGGGACCAAGGTGGAAAT | |
| | TACTGGGGCCAGGGAACCCTGGTCA | | CAAACGAACTGTGGCTGCACCATC | |
| | CCGTCTCCTCAGCCTCCACCAAGGG | | TGTCTTCATCTTCCCGCCATCTGAT | |
| | CCCATCGGTCTTCCCCCTGGCACCCT | | GAGCAGTTGAAATCTGGAACTGCC | |
| | CCTCCAAGAGCACCTCTGGGGGCAC | | TCTGTTGTGTGCCTGCTGAATAACT | |
| | AGCGGCCCTGGGCTGCCTGGTCAAG | | TCTATCCCAGAGAGGCCAAAGTAC | |
| | GACTACTTCCCCGAACCGGTGACGG | | AGTGGAAGGTGGATAACGCCCTCC | |
| | TGTCGTGGAACTCAGGCGCCCTGAC | | AATCGGGTAACTCCCAGGAGAGTG | |
| | CAGCGGCGTGCACACCTTCCCGGCT | | TCACAGAGCAGGACAGCAAGGACA | |
| | GTCCTACAGTCCTCAGGACTCTACT | | GCACCTACAGCCTCAGCAGCACCC | |
| | CCCTCAGCAGCGTGGTGACCGTGCC | | TGACGCTGAGCAAAGCAGACTACG | |
| | CTCCAGCAGCTTGGGCACCCAGACC | | AGAA | |
| | TACATCTGCAACGTGAATCACAAGC | | | |
| | CCACCTTGGTGTTGCTGGGCTTGTG | | | |
| | ATTCAC | | | |
| S2141-16 | CAGGTGCAGTTACAGCAGTGGGGCG | 2734 | TCCTATGAACTGACTCAGTCACTCT | 2783 |
| | CAGGACTGTTGAAGCCTTCGGAGAC | | CAGTGTCAGTGGCCCTGGGACAGA | |
| | CCTGTCCCGCACCTGCGCTGTCTAT | | CGGCCAGAATTCCCTGTGGGGGAA | |
| | GGTGGGTCCTTCAGTGGTTACTACT | | ACAACATTGGAAGTAAAAATGTGC | |
| | GGAGCTGGATCCGCCAGACCCCAGG | | ACTGGTACCAGCAGAAGCCAGGCC | |
| | GAAGGGGCTGGAGTGGATTGGGGA | | AGGCCCCTGTGCTGGTCATCTACA | |
| | AATCAATCATGATGGAAGCACCATC | | GCGATCGCAACCGGCCCTCTGGGA | |
| | TACAACCCGTCCCTCAAGAGTCGAG | | TCCCTGAGCGATTCTCAGGCTCCAA | |
| | TCACCATATCGATAGACACGTCCAA | | CTCGGGGAACACGGCCACCCTGAC | |
| | GAACCAGTTCTCCCTGCAACTGAGC | | CATCAGCAGAGCCCAAGCCGGGGA | |
| | TCTGTGACCGCCGCGGACACGGCTG | | TGAGGCTGACTATTACTGTCAGGT | |
| | TGTACTACTGTGCGAGAGGGTCTAA | | GTGGGACAGTAGCTCTGTGGTATT | |
| | TCCTGGGGACTACTGGGGCCAGGGA | | CGGCGGAGGGACCAAGCTGACCGT | |
| | GCCCTGGTCACCGTCTCCTCAGCAC | | CCTACGTCAGCCCAAGGCTGCCCC | |
| | CCACCAAGGCTCCGGATGTGTTCCC | | CTCGGTCACTCTGTTCCCGCCCTCC | |
| | CATCATATCAGGGTGCAGACACCCA | | TCTGAGGAGCTTCAAGCCAACAAG | |
| | AAGGATAACAGCCCTGTGGTCCTGG | | GCCACACTGGTGTGTCTCATAAGT | |
| | CATGCTTGATAACTGGGTACCACCC | | GACTTCTACCCGGGAGCCGTGACA | |
| | AACGTC | | GTGGCCTGGAAGGCAGATAGCAGC | |
| | | | CCCGTCAAGGCGGGAGTGGAGACC | |
| | | | ACCACACCCTCCAAACAAAGCAAC | |
| | | | AACAAGTACGCGGCCAGCAGCTAT | |
| | | | CTGAGCCTGACGCCTGAGCAGTGG | |
| | | | AAGTCCCACA | |
| S2141-62 | CAGGTGCACCTGCAGGAGTCGGGCC | 2735 | CAGTCTGCCCTGACTCAGCCTACCT | 2784 |
| | CAGGACTGGTGAAGCCTTCACAGAC | | CCGTGTCTGGGTCTCCTGGACAGTC | |
| | CCTGTCCCTCACTTGCACTGTCTCTG | | GATCACCATCTCCTGCACTGGAAC | |
| | GTGTCTCCATCACCACTAGTGGCTC | | CAGCAGTGATGTTGGGCGTTATAA | |
| | CTACTGGAGCTGGATCCGCCAGTGC | | CCTTGTCTCCTGGTACCAACAGTAC | |
| | CCAGGGAAGGGCCTGGAGTGGATT | | CCAGGCAAAGCCCCCAAACTCATC | |
| | GGATACATCTATTCCACTGGGACCA | | ATTTTTGAGGTCAGTAAGCGGCCCT | |
| | CCTACTACAGTCCGTCCCTCAAGAG | | CAGGGGTCTCTGATCGCTTCTCTGC | |
| | TCGACTTACCATATCCCTAGACACG | | CTCAAAGTCTGGCAACACGGCCTC | |
| | TCTAGGAACCAATTCTCCCTGAACC | | CCTGACAATCTCTGGGCTCCAGGCT | |
| | TGAGTTCTGTGACTGCCGCGGACAC | | GACGACGAGGCTGATTATTACTGC | |
| | GGCCGTGTTTTTCTGTGCTAGAAAA | | TGCACATATGCTCTTACATTTTTGT | |
| | ACCTACATGGACTACTTTGACTACT | | TCGGCGGAGGGACCAAAGTGACCG | |
| | GGGGCCAGGGAGCCCTGATCACCGT | | TCCTAGGTCAGCCCAAGGCTGCCC | |
| | CTCCTCAGCCTCCACCAAGGGCCCA | | CCTCGGTCACTCTGTTCCCGCCCTC | |
| | TCGGTCTTCCCCCTGGCACCCTCCTC | | CTCTGAGGAGCTTCAAGCCAACAA | |
| | CAAGAGCACCTCTGGGGGCACAGC | | GGCCACACTGGTGTGTCTCATAAG | |
| | GGCCCTGGGCTGCCTGGTCAAGGAC | | TGACTTCTACCCGGGAGCCGTGAC | |
| | TACTTCCCCGAACCGGTGACGGTGT | | AGTGGCCTGGAAGGCAGATAGCAG | |
| | CGTGGAACTCAGGCGCCCTGACCAG | | CCCCGTCAAGGCGGGAGTGGAGAC | |
| | CGGCGTGCACACCTTCCCGGCTGTC | | CACCACACCCTCCAAACAAAGCAA | |
| | CTACAGTCCTCAGGA | | CAACAAGTACGCGGCCAGCAGCTA | |
| | | | TCTGAGCCTGACGCCTGAGCAGTG | |
| | | | GAAGTCCCAC | |
| S2141-63 | GAGGTGCAGTTGTTGGAGTCTGGGG | 2736 | GACATCCAGATGACCCAGTCTCCA | 2785 |
| | GAGGCTTGGTACAGCCTGGGGGGTC | | TCCTCCCTGTCTGCATCTGTAGGAG | |
| | CCTGAGACTCTCCTGTGCAGCCTCT | | ACAGGGTCACCATCACTTGCCGGT | |
| | GGATTCACCTTTTACGACTATGCCA | | CAGGTCAGAGCATTAGCACCTATT | |
| | TGAACTGGGTCCGCCAGACTCCAGG | | TAAATTGGTATCAGCAGAAACCAG | |
| | GGAGGGGCTGGAGTGGGTCTCAGCC | | GAAAAGCCCCTAAACTCCTGATCT | |
| | ATTAGTGGCAGTGGTGATCCCACAT | | ATGCTTCATCCAGTTTGCAAAGTGG | |
| | ACTACGCAGACTCCGTGAACGGCCG | | GGTCCCATCAAGGTTCAGTGGCAG | |
| | CTTCACCATCTCCAGAGACAATTCC | | TGGATCTGGGACAGATTTCACTCTC | |
| | AAGAACACACTGTATCTGCAAATGA | | ACCATCAGCAGTCTGCAACCTGAA | |
| | ACAGTCTGAGAGCCGAGGATACGG | | GATTTTGCAACTTACTACTGTCAAC | |
| | CCATATATTATTGTGCGAAAGACAT | | AGAGTTTCCTTCCCCCTCGAACTTT | |
| | GGAGGACTTCGGTTTTAGTTGGGGC | | TGGCCAGGGAACCAAGCTGGAGAT | |
| | CAGGGAACCCTGGTCACCGTCTCCT | | CAAACGAACTGTGGCTGCACCATC | |
| | CAGCACCCACCAAGGCTCCGGATGT | | TGTCTTCATCTTCCCGCCATCTGAT | |
| | GTTCCCCATCATATCAGGGTGCAGA | | GAGCAGTTGAAATCTGGAACTGCC | |
| | CACCCAAAGGATAACAGCCCTGTGG | | TCTGTTGTGTGCCTGCTGAATAACT | |
| | TCCTGGCATGCTTGATAACTGGGTA | | TCTATCCCAGAGAGGCCAAAGTAC | |
| | CCACCCAACGTCCGTGACTGTCACC | | AGTGGAAGGTGGATAACGCCCTCC | |
| | TGGTACATGGG | | AATCGGGTAACTCCCAGGAGAGTG | |
| | | | TCACAGAGCAGGACAGCAAGGACA | |
| | | | GCACCTACAGCCTCAGCAGCACCC | |
| | | | TGACGCTGAGCAAAGCAGACTACG | |
| | | | AGAA | |
| S2141-65 | GACGTGCAGCTGGTGCAGTCTGGAG | 2737 | GACATCCAGATGACCCAGTCTCCTT | 2786 |
| | CAGAGGTGACAAAGCCGGGGGAGT | | CCACCCTGTCTGCATCTGTAGGAG | |
| | CTCTGAAGATCTCCTGTAAGGGTTC | | ACAGAGTCACCATCACTTGCCGGG | |
| | TGGATACAGCTTTACCACCTACTGG | | CCAGTCAGAGTATTAGTAGCTGGT | |
| | ATCGGCTGGGTGCGCCAGATGCCCG | | TGGCCTGGTATCAGCAGAAACCAG | |
| | GGAAAGGCCTGGAGTGGATGGGGA | | GGAAAGCCCCTAAGCTCCTGATCT | |
| | TCATCTATCCTGGTGACTCTGATACC | | ATGATGCCTCCAGTTTGGAAGGTG | |
| | AGATACAGCCCGTCCTTCCAAGGCC | | GGGTCCCATCAAGGTTCAGCGGCA | |
| | AGGTCACCATCTCAGTCGACAAGTC | | GTGGATCTGGGACAGAATTCACTC | |
| | CATCAGCACCGCCTACCTGCAGTGG | | TCACCATCAGCAGCCTGCAGCCTG | |
| | AGCAGCCTGAAGGCCTCGGACACCG | | ATGATTTTGCAACTTATTACTGCCA | |
| | CCATGTATTACTGTGCGAGACAGTT | | ACAGTATAATAGTTATCCCCGGAC | |
| | TTGTGGTGGTGACTGCCCCTTTGACT | | GTTCGGCCAAGGGACCAAGGTGGA | |
| | ACTGGGGCCGGGGAACCCTGGTCAC | | AATCAAACGAACTGTGGCTGCACC | |
| | CGTCTCCTCAGCTTCCACCAAGGGC | | ATCTGTCTTCATCTTCCCGCCATCT | |
| | CCATCGGTCTTCCCCCTGGCGCCCT | | GATGAGCAGTTGAAATCTGGAACT | |
| | GCTCCAGGAGCACCTCTGGGGGCAC | | GCCTCTGTTGTGTGCCTGCTGAATA | |
| | AGCGGCCCTGGGCTGCCTGGTCAAG | | ACTTCTATCCCAGAGAGGCCAAAG | |
| | GACTACTTCCCCGAACCGGTGACGG | | TACAGTGGAAGGTGGATAACGCCC | |
| | TGTCGTGGAACTCAGGCGCCCTGAC | | TCCAATCGGGTAACTCCCAGGAGA | |
| | CAGCGGCGTGCACACCTTCCCGGCT | | GTGTCACAGAGCAGGACAGCAAGG | |
| | GTCCTACAGTCCTCAGGACTCTACT | | ACAGCACCTACAGCCTCAGCAGCA | |
| | CCCTCAGCAGCGTGGTGACCGTGCC | | CCCTGACGCTGAGCAAAGCAGACT | |
| | CTCCAGCAGCTTGGGCACCCAGACC | | ACGAGAA | |
| | TACACCTGCAACGTGAATCACAAGC | | | |
| | CCAGCAACACCAAGGTGGACAA | | | |
| S2141-97 | CAGGTCCAGCTTGTGCAGTCTGGGG | 2738 | GAAATTGTGTTGACGCAGTCTCCA | 2787 |
| | CTGAGGTGAAGAAGCCTGGGGCCTC | | GGCACCCTGTCTTTGTCTCCAGGGG | |
| | AGTGAAGGTTTCCTGCAAGGCTTCT | | AAAGAGCCACCCTCTCCTGCAGGG | |
| | GGATACACCTTCACTAGATATGGTA | | CCAGTCAGAGAGTTAGCAGCAGCT | |
| | TGCATTGGGTGCGCCAGGCCCCCGG | | ACATAGCCTGGTACCAGCAGAAAC | |
| | ACAAAGGCTTAAGTGGATGGGATG | | CTGGCCAGGCTCCCAGGCTCCTCAT | |
| | GATCAACGCTGGCAATGGTAACACA | | CTTTGGTACATCCAGCAGGGCCAC | |
| | AAATATTCACAGAAGTTCCAGGGCA | | TGGCATCCCAGACAGGTTCAGTGG | |
| | GACTCACCATTAGCAGGGACACATC | | CAGTGGGTCCGGGACAGACTTCAC | |
| | CGCGAGCACAGCCTACATGGAGGTG | | TCTCACCATCAGCAGACTGGAGCC | |
| | AGCAGTCTGAGATCTGAAGACACGG | | TGAAGATTTTGCACTGTATTACTGT | |
| | CTGTGTATTACTGTGCGAGATCGGG | | CAACAGTATGGTAGCTCACCGTAC | |
| | TATAGCAGCAGCTGGTAGTAAAGTA | | ACTTTTGGCCAGGGGACCAAGCTG | |
| | ATCTACTACTACGATATGGACGTCT | | GAGATCAAACGAACTGTGGCTGCA | |
| | GGGGCCAAGGGACCACGGTCACCG | | CCATCTGTCTTCATCTTCCCGCCAT | |
| | TCTCCTCAGCACCCACCAAGGCTCC | | CTGATGAGCAGTTGAAATCTGGAA | |
| | GGATGTGTTCCCCATCATATCAGGG | | CTGCCTCTGTTGTGTGCCTGCTGAA | |
| | TGCAGACACCCAAAGGATAACAGC | | TAACTTCTATCCCAGAGAGGCCAA | |
| | CCTGTGGTCCTGGCATGCTTGATAA | | AGTACAGTGGAAGGTGGATAACGC | |
| | CTGGGTACCACCCAACGTCCGTGAC | | CCTCCAATCGGGTAACTCCCAGGA | |
| | TGTCACCTGGTACATGGGGACACAG | | GAGTGTCACAGAGCAGGACAGCAA | |
| | AGCCAGCCCCAGAGAACCTTCCCTG | | GGACAGCACCTACAGCCTCAGCAG | |
| | AGATACAAAGACGGGACAGCTACT | | CACCCTGACGCTGAGCAAAGCAGA | |
| | ACATGACAAGCAGCCAGCTCTCCAC | | CTACGAGAA | |
| | CCCCCTCCAGCAGTGGCGCCAAGGC | | | |
| | GAGTACAAATGCGTGGTCCAGCA | | | |
| S24_342 | CAGGTGCAACTGGTGCAGTCTGGGG | 2739 | CACTCTGCCCTGACTCAGCCTCCCT | 2788 |
| | CTGAGGTGAAGATGCCTGGGGCCTC | | CCGCGTCTGGGTCTCCTGGACAGTC | |
| | AGTGATTGTTTCCTGCAAGGCATCT | | AGTCACCATTTCCTGCACTGGAACC | |
| | GGATACACCTTCAGCACCTACTATA | | AGCAGTGACGTTGGTGGTTATAAC | |
| | TTCACTGGGTGCGACAGGCCCCTGG | | CATGTCTCCTGGTACCAACAGCAC | |
| | ACAAGGGCTTGAGTGGATGGGAAG | | CCAGGCAAAGCCCCCAAATTAATG | |
| | AATCACCCCCCGCGATGGTGACACA | | GTTTATGAGGTCAATCAGCGGCCC | |
| | ACCTACGCACAGGTGTTGCAGGGCA | | TCAGGGGTCCCTGATCGCTTCACTG | |
| | GAGTCACATTGACCAGGGACACGTC | | GCTCCAAGTCTGGCAACACGGCCT | |
| | CGCGAGCACAGCCTACATGGAGCTG | | CCCTGACCGTCTCTGGGCTCCAGGC | |
| | AGCAGCCTGACATATGAGGACACG | | TGAGGATGAGGCTGATTATTATTG | |
| | GCCGTCTATTATTGTGCGAGAGATG | | CAACTCATATACAGACAGGAACAA | |
| | GACATCACTGGGACTTTGACTTCTG | | GTGGGTGTTCGGCGGAGGGACCAG | |
| | GGGCCGGGGAACCCTGGTCGCCGTC | | GCTGACCGTCCTAGGTCAGCCCAA | |
| | TCCTCAGCCTCCACCAAGGGCCCAT | | GGCTGCCCCCTCGGTCACTCTGTTC | |
| | CGGTCTTCCCCCTGGCGCCCTGCTCC | | CCGCCCTCCTCTGAGGAGCTTCAA | |
| | AGGAGCACCTCCGAGAGCACAGCG | | GCCAACAAGGCCACACTGGTGTGT | |
| | GCCCTGGGCTGCCTGGTCAAGGACT | | CTCATAAGTGACTTCTACCCGGGA | |
| | ACTTCCCCGAACCGGTGACGGTGTC | | GCCGTGACAGTGGCCTGGAAGGCA | |
| | GTGGAACTCAGGCGCCCTGACCAGC | | GATAGCAGCCCCGTCAAGGCGGGA | |
| | GGCGTGCACACCTTCCCGGCTGTCC | | GTGGAGACCACCACACCCTCCAAA | |
| | TACAGTCCTCAGG | | CAAAGCAACAACAAGTACGCGGCC | |
| | | | AGCAGCTA | |
| S24-1047 | CAGGTGCAGCTGAAGCAGTCTGGGG | 2740 | CACTCTGCCCTGACTCAGCCTCCCT | 2789 |
| | CTGAGGTGAAGGAGCCCGGGGGCT | | CCGCGTCTGGGTCTCCTGGACAGTC | |
| | CAGTGAAGCTTTCCTGCAAGGCGTC | | AGTCACCATTTCCTGCACTGGAACC | |
| | TGGATACACCTTCACCTCCCGCTAT | | AGCGATGACGTTGGTGGTTATAAC | |
| | ATACACTGGGTGCGACAGGCCCCTG | | CATGTCTCCTGGTATCAACAGCACC | |
| | GACAAGGGCTTGAGTGGGTGGGAA | | CAGGCAAAGCCCCCAAATTAGTGA | |
| | GACTTATTCCCAGTGACGGTGGCAC | | TTTATGAGGTCACTGAGCGGCCCTC | |
| | AACCTACGCACAGAAATTTCGCGGC | | AGGGGTCCCTGATCGCTTCACTGG | |
| | AGAGTCACCATGACCAGCGACACGT | | CTCCAAGTCTGGCAACACGGCCTC | |
| | CCGCGACCACAGCCTACATGGAGCT | | CCTGACCGTCTCTGGGCTCCAGGCT | |
| | GAGCAGCCTTGGATCTGGCGACACG | | GAGGATGAGGCTGATTATTACTGC | |
| | GCCGTCTATTACTGTGCGCGAGACG | | AACTCATATAAAAGGGGCAACACT | |
| | GGACTCACTGGGACTTTGACTTCTG | | TGGGTGTTCGGCGGAGGGACCAGG | |
| | GGGCCAGGGAACCCTGGTCACCGTC | | CTGACCGTCCTAGGTCAGCCCAAG | |
| | TCCTCTGCATCCCCGACCAGCCCCA | | GCTGCCCCCTCGGTCACTCTGTTCC | |
| | AGGTCTTCCCGCTGAGCCTCGACAG | | CGCCCTCCTCTGAGGAGCTTCAAG | |
| | CACCCCCCAAGATGGGAACGTGGTC | | CCAACAAGGCCACACTGGTGTGTC | |
| | GTCGCATGCCTGGTCCAGGGCTTCT | | TCATAAGTGACTTCTACCCGGGAG | |
| | TCCCCCAGGAGCCACTCAGTGTGAC | | CCGTGACAGTGGCCTGGAAGGCAG | |
| | CTGGAGCGAAAGCGGACAGAACGT | | ATAGCAGCCCCGTCAAGGCGGGAG | |
| | GACCGCCAGAAACTTCCC | | TGGAGACCACCACACCCTCCAAAC | |
| | | | AAAGCAACAACAAGTACGCGGCCA | |
| | | | GCAGCTA | |
| S24-223 | CAGATCACCTTGAAGGAGTCTGGTC | 2741 | CAGTCTGCCCTGACTCAGCCTGCCT | 2790 |
| | CTACGCTGGTGAAACCCACACAGAC | | CCGTGTCTGGGTCTCCTGGACAGTC | |
| | CCTCACGCTGACCTGCACCTTCTCTG | | GATCACCATCTCCTGCACTGGAAC | |
| | GGTTCTCACTCAACACTAGTGGAGT | | CAGCAGTGACGTTGGTGGTTATAA | |
| | GGGTGTGGGCTGGATCCGTCAGCCC | | CTATGTCTCCTGGTACCAACAACAC | |
| | CCAGGAAAGGCCCTGGAGTGGCTTG | | CCAGGCAAAGCCCCCAAACTCATG | |
| | CACTCATTTATTGGGATGATGATAA | | ATTTATGATGTCAGTAATCGGCCCT | |
| | GCGCTACAGCCCATCTCTGAAGAGC | | CAGGGGTTTCTAATCGCTTCTCTGG | |
| | AGGCTCACCATCACCAAGGACACCT | | CTCCAAGTCTGGCAACACGGCCTC | |
| | CCAAAAACCAGGTGGTCCTTACAAT | | CCTGACCATCTCTGGGCTCCAGGCT | |
| | GACCAACATGGACCCTGTGGACACA | | GAGGACGAGGCTGATTATTACTGC | |
| | GCCACATATTACTGTGCACACCATA | | AACTCATATACAAGCAGCAGCACT | |
| | CGATTGTTCCAATTTTTGACTACTGG | | CTCGTGGTATTCGGCGGAGGGACC | |
| | GGCCAGGGAACCCTGGTCACCGTCT | | AAGCTGACCGTCCTAGGTCAGCCC | |
| | CCTCAGGGAGTGCATCCGCCCCAAC | | AAGGCTGCCCCCTCGGTCACTCTGT | |
| | CCTTTTCCCCCTCGTCTCCTGTGAGA | | TCCCGCCCTCCTCTGAGGAGCTTCA | |
| | ATTCCCCGTCGGATACGAGCAGCGT | | AGCCAACAAGGCCACACTGGTGTG | |
| | G | | TCTCATAAGTGACTTCTACCCGGGA | |
| | | | GCCGTGACAGTGGCCTGGAAGGCA | |
| | | | GATAGCAGCCCCGTCAAGGCGGGA | |
| | | | GTGGAGACCACCACACCCTCCAAA | |
| | | | CAAAGCAACAACAAGTACGCGGCC | |
| | | | AGCAGCTATCTGAGCCTGACGCC | |
| S24-237 | CAGGTGCAGCTGCAGGAGTCGGGCC | 2742 | GACATCGTGATGACCCAGTCTCCA | 2791 |
| | CAGGACTGGTGAAGCCTTCGGGGAC | | GACTCCCTGGCTGTGTCTCTGGGCG | |
| | CCTGTCCCTCACCTGCTCTGTCTCTG | | AGAGGGCCACCATCAACTGCAAGT | |
| | GTGGCTCCATCAATAGTTCCTTCTG | | CCAGCCAGACTGTTTCATACACCTC | |
| | GAGCTGGATCCGGCAGCCCCCAGGG | | CAACAATAAGAACTACCTAGCTTG | |
| | AAGGGACTGGAGTGGATTGGGTATA | | GTACCAGCAGAAACCAGGACAGCC | |
| | TCTATTACCGTGGGAGCACCAATTA | | TCCTAACCTGCTCATTTACTGGGCA | |
| | CAACCCCTCCCTCAAGAGTCGAGTC | | TCTACCCGGGAATCCGGGGTCCCT | |
| | ACCATATCAGTGGACACGTCCAACA | | GACCGATTCAGTGGCAGCGGGTCT | |
| | ATCAGTTCTCCCTGAAGCTGACCTC | | GGGACAGATTTCACTCTCACCATC | |
| | TATGACCGCTGCGGACTCGGCCGTG | | AACAGCCTGCAGGCTGAAGATGTG | |
| | TATTACTGTGCGCGAGAAACCCGAT | | GCAGTTTATTACTGTCAGCAATATT | |
| | ACAACTGGTTCGACTCCTGGGGCCA | | ATACTACTCCGTGGACGTTCGGCC | |
| | GGGAACCCGGGTCACCGTCTCCTCA | | AAGGGACCAAGGTGGAAATCAAAC | |
| | GCCTCCACCAAGGGCCCATCGGTCT | | GAACTGTGGCTGCACCATCTGTCTT | |
| | TCCCCCTGGCGCCCTGCTCCAGGAG | | CATCTTCCCGCCATCTGATGAGCAG | |
| | CACCTCCGAGAGCACAGCGGCCCTG | | TTGAAATCTGGAACTGCCTCTGTTG | |
| | GGCTGCCTGGTCAAGGACTACTTCC | | TGTGCCTGCTGAATAACTTCTATCC | |
| | CCGAACCGGTGACGGTGTCGTGGAA | | CAGAGAGGCCAAAGTACAGTGGAA | |
| | CTCAGGCGCCCTGACCAGCGGCGTG | | GGTGGATAACGC | |
| | CACACCTTCCCGGCTGTCCTACAGT | | | |
| | CCTCAGGA | | | |
| S305-1456 | CAGGTCCAGCTGGTACAGTCTGGGG | 2743 | GAAATAGTGATGACGCAGTCTCCA | 2792 |
| | CTGAGGTGAAGAAGCCTGGGGCCTC | | GCCACCCTGTCTGTGTCTCCAGGGG | |
| | AGTGAAGGTCTCCTGCAAGGTTTCC | | AAAGAGCCACCCTCTCCTGCAGGG | |
| | GGATACACCCTCACTGAATTATCCA | | CCAGTCAGAATGTTAGCAGCAACT | |
| | TGCACTGGGTGCGGCAGGCTCCTGG | | TAGCCTGGTACCAACAGAAACCTG | |
| | AAAAGGGCTTGAGTGGATGGGAGG | | GCCAGGCTCCCAGGCTCCTCATCTA | |
| | TTTTGATCCTGAAGATGCTGAAACA | | TGGTGCATCCACCAGGGCCACTGG | |
| | ATCTACGCACAGAAGTTCCAGGGCA | | TATCCCGGCCAGGTTCAGTGGCAG | |
| | GAGTCACCATGACCGAGGACACATC | | TGGGTCTGGGACAGAGTTCACTCT | |
| | TACAGACACAGCCTACATGGAGCTG | | CACCATCAGCAGCCTGCAGTCTGA | |
| | AGCAGCCTGAGATCTGAGGACACG | | AGATTTTGCAGTTTATTACTGTCAG | |
| | GCCGTGTATTACTGTGCAACAGGGG | | CAGTATAATAACTGGCCTCACACTT | |
| | GCTTTCCCGTCAATAGCCTTTACGAT | | TCGGCCCTGGGACCAAAGTGGATA | |
| | ATTTTGACTGGTTACCTTGACTACTG | | TCAAACGAACTGTGGCTGCACCAT | |
| | GGGCCAGGGAACCCTGGTCACCGTC | | CTGTCTTCATCTTCCCGCCATCTGA | |
| | TCCTCAGCCTCCACCAAGGGCCCAT | | TGAGCAGTTGAAATCTGGAACTGC | |
| | CGGTCTTCCCCCTGGCGCCCTGCTCC | | CTCTGTTGTGTGCCTGCTGAATAAC | |
| | AGGAGCACCTCCGAGAGCACAGCG | | TTCTATCCCAGAGAGGCCAAAGTA | |
| | GCCCTGGGCTGCCTGGTCAAGGACT | | CAGTGGAAGGTGGATAACGC | |
| | ACTTCCCCGAACCGGTGACGGTGTC | | | |
| | GTGGAACTCAGGCGCTCTGACCAGC | | | |
| | GGCGTGCACACCTTCCCAGCTGTCC | | | |
| | TACAGTCCTCAGGA | | | |
| S305-223 | CAGGTGCAGTTGGTGGAGTCTGGGG | 2744 | GAAATTGTGTTGACACAGTCTCCA | 2793 |
| | GAGGCGTGGTCCAGCCTGGAAGGTC | | GCCACCCTGTCTTTGTCTCCAGGGG | |
| | CCTGAGACTCTCCTGTGCAGCGTCT | | AAAGAGCCACCCTCTCCTGCAGGG | |
| | GGATTCACCTTCAGAAACTTTGGCA | | CCAGTCAGAGTGTTAGCACCTCCTT | |
| | TGCACTGGGTCCGCCAGGCTCCAGG | | AGCCTGGTACCAACAGAAATGTGG | |
| | CAAGGGGCTGGAGTGGGTGGCATTT | | CCAGGCTCCCCGGCTCCTCATCTAT | |
| | ATATGGACTGCTGAAAGTGATAAAT | | GATGCATCCAACAGGGCCACTGGC | |
| | TCTATGCAGACTCCGTGAAGGGCCG | | ATCCCAGCCAGGTTCAGTGGCAGT | |
| | ATTCACCGTCTCCAGAGACAATTCG | | GGGTCTGGGACAGACTTCACTCTC | |
| | AAGAACACGCTGTATTTGGAAATGA | | ACCATCAGCAGCCTAGAGCCTGAA | |
| | ACAGCCTGAGAGCCGAGGACACGG | | GATTTTGCAGTTTATTACTGTCAAC | |
| | CTGTGTATTACTGTACGAAAGCGAT | | AGCGTGGCAACTGGCCCTTCACTTT | |
| | GGACGTCTGGGGCAGAGGGACCAC | | CGGCCCTGGGACCAGAGTGGATAT | |
| | GGTCACCGTCTCCTCAGCATCCCCG | | CAAACGAACTGTGGCTGCACCATC | |
| | ACCAGCCCCAAGGTCTTCCCGCTGA | | TGTCTTCATCTTCCCGCCATCTGAT | |
| | GCCTCTGCAGCACCCAGCCAGATGG | | GAGCAGTTGAAATCTGGAACTGCC | |
| | GAACGTGGTCATCGCCTGCCTGGTC | | TCTGTTGTGTGCCTGCTGAATAACT | |
| | CAGGGCTTCTTCCCCCAGGAGCCAC | | TCTATCCCAGAGAGGCCAAAGTAC | |
| | TCAGTGTGACCTGGAGCGAAAGCGG | | AGTGGAAGGTGGATAACGC | |
| | ACAGGGCGTGACCGCCAGAAACTTC | | | |
| | CC | | | |
| S305-399 | CAGGTCCAGCTGGTACAGTCTGGGG | 2745 | GAAATAGTGATGACGCAGTCTCCA | 2794 |
| | CTGAGGTGAAGAAGCCTGGGGCCTC | | GCCACCCTGTCTGTGTCTCCAGGGG | |
| | AGTGAAGGTCTCCTGCAAGGTTTCC | | AAAGAGCCACCCTCTCCTGCAGGG | |
| | GGATACACCCTCACTGAATTATCCA | | CCAGTCAGAGTATTACTAGCAACT | |
| | TGCACTGGGTGCGACAGGCTCCTGG | | TAGCCTGGTACCAGCAGAAACCTG | |
| | AAAAGGGCTTGAGTGGATGGGAGG | | GCCAGGCTCCCAGGCTCCTCATCTA | |
| | TTTTGATCCTGAAGATGGTGAAACA | | TGGTGCATCCACCAGGGCCACTGG | |
| | ATCTACGCACAGAAGTTCCAGGGCA | | TATCCCAGCCAGGTTCAGTGGCAG | |
| | GAGTCACCATGACCGAAGACACATC | | TGGGTCTGGGACAGAGTTCACTCT | |
| | TACAGACACAGCCTACATGGAGCTG | | CACCATCAGCAACCTGCAGTCTGA | |
| | AGCAGCCTGAGATCTGAGGACACG | | AGATTTTGCAGTTTATTACTGTCAG | |
| | GCCGTGTATTACTGTGCAACAGGGG | | CAGTATAATAACTGGCCTCTGACG | |
| | GATTGGGTTGTTCTAATGGGGTATG | | TTCGGCCAAGGGACCAAGGTGGAA | |
| | CAACAACTGGTTCGACCCCTGGGGC | | ATCAAACGAACTGTGGCTGCACCA | |
| | CTGGGAACCCTGGTCACCGTCTCCT | | TCTGTCTTCATCTTCCCGCCATCTG | |
| | CAGGGAGTGCATCCGCCCCAACCCT | | ATGAGCAGTTGAAATCTGGAACTG | |
| | TTTCCCCCTCGTCTCCTGTGAGAATT | | CCTCTGTTGTGTGCCTGCTGAATAA | |
| | CCCCGTCGGATACGAGCAGCGTG | | CTTCTATCCCAGAGAGGCCAAAGT | |
| | | | ACAGTGGAAGGTGGATAACGCCCT | |
| | | | CCAATCGGGTAACTCCCAGGAGAG | |
| | | | TGTCACAGAGCAGGACAGCAA | |
| S305-968 | GAGGTGCAGCTGGTGGAGTCTGGGG | 2746 | TCCTATGAGCTGACTCAGCCACCCT | 2795 |
| | GAGGCTTGGTCCAGCCTGGGGGGTC | | CAGTGTCCGTGTCCCCAGGACAGA | |
| | CCTGAGACTCTCCTGTGCAGCCTCT | | CAGCCAGCATCACCTGCTCTGGAG | |
| | GGATTCACCTTTAGTAGCTATTGGA | | ATAAATTGGGGGATAAATATGCTT | |
| | TGAGCTGGGTCCGCCAGGCTCCAGG | | GCTGGTATCAGCAGAAGCCAGGCC | |
| | GAAGGGGCTGGAGTGGGTGGCCAA | | AGTCCCCTGTGCTGGTCATCTATCA | |
| | CATAAAGCAAGATGGAAGTGAGAA | | AGATAGCAAGCGGCCCTCAGGGAT | |
| | ATACTATGTGGACTCTGTGAAGGGC | | CCCTGAGCGATTCTCTGGCTCCAAC | |
| | CGATTCACCATCTCCAGAGACAACG | | TCTGGGAACACAGCCACTCTGACC | |
| | CCAAGAACTCACTGTATCTGCAAAT | | ATCAGCGGGACCCAGGCTATGGAT | |
| | GAACAGCCTGAGAGCCGAGGACAC | | GAGGCTGACTATTACTGTCAGGCG | |
| | GGCCGTGTATTACTGTGCGAGGGAT | | TGGGACAGCAGCACTAATGTGGTA | |
| | AGTATAGCAGTGGCTGGGGGCTTTG | | TTCGGCGGAGGGACCAAGCTGACC | |
| | ACTACTGGGGCCAGGGAACCCTGGT | | GTCCTAGGTCAGCCCAAGGCTGCC | |
| | CACCGTCTCCTCAGGGAGTGCATCC | | CCCTCGGTCACTCTGTTCCCGCCCT | |
| | GCCCCAACCCTTTTCCCCCTCGTCTC | | CCTCTGAGGAGCTTCAAGCCAACA | |
| | CTGTGAGAATTCCCCGTCGGATACG | | AGGCCACACTGGTGTGTCTCATAA | |
| | AGCAGCGTG | | GTGACTTCTACCCGGGAGCCGTGA | |
| | | | CAGTGGCCTGGAAGGCAGATAGCA | |
| | | | GCCCCGTCAAGGCGGGAGTGGAGA | |
| | | | CCACCACACCCTCCAAACAAAGCA | |
| | | | ACAACAAGTACGCGGCCAGCAGCT | |
| | | | ACCTGAGCCTGACGCCTGAGCAGT | |
| | | | GGAAGTCCCAC | |
| S376-1070 | CAGGTGCAGCTGGTGGAGTCTGGGG | 2747 | CAGTCTGCCCTGACTCAGCCTCGCT | 2796 |
| | GAGGCGTGGTCCAGCCTGGGAGGTC | | CAGTGTCCGGGTCTCCTGGACAGT | |
| | CCTGAGACTCTCCTGTGCAGCGTCT | | CAGTCACCATCTCCTGCACTGGAA | |
| | GGATTCACCTTCAGTAGCTATGGCA | | GCAGCAGTGATGTTGGTCGTTATA | |
| | TGCACTGGGTCCGCCAGGCTCCAGG | | ACTATGTCTCCTGGTACCAGCAAC | |
| | CAAGGGGCTGGAGTGGGTGGCAGTT | | ACCCAGGCAAAGCCCCCAAACTCA | |
| | ATATGGTATGATGGAAGTAATAAAT | | TGACTTATGATGTCACTAGGCGGC | |
| | ACTATGCAGACTCCGTGAAGGGCCG | | CCTCAGGGGTCCCTGCTCGCTTCTC | |
| | ATTCACCATCTCCAGAGACAATTCC | | TGGCTCCAAGTCTGACAACACGGC | |
| | AAGAACACGCTGTATCTGCAAATGA | | CTCCCTGACCATCTCTGGGCTCCAG | |
| | ACAGCCTGAGAGCCGAGGACACGG | | GCTGAGGATGAGGCCGATTATTAT | |
| | CTGTGTATTACTGTGCGAGGATGCG | | TGTTGCTCATTTGCAGGCAGCTACA | |
| | TCCTGAATATTCCAGCGGGTTCGAC | | CTGTGTTCGGCGGAGGGACCAAAC | |
| | CCCTGGGGCCAGGGAACCCTGGTCA | | TGACCGTCCTGGGTCAGCCCAAGG | |
| | CCGTCTCCTCAGGGAGTGCATCCGC | | CTGCCCCCTCGGTCACTCTGTTCCC | |
| | CCCAACCCTTTTCCCCCTCGTCTCCT | | GCCCTCCTCTGAGGAGCTTCAAGC | |
| | GTGAGAATTCCCCGTCGGATACGAG | | CAACAAGGCCACACTGGTGTGTCT | |
| | CAGCGTG | | CATAAGTGACTTCTACCCGGGAGC | |
| | | | CGTGACAGTGGCCTGGAAGGCAGA | |
| | | | TGGCAGCCCCGTCAAGGTGGGAGT | |
| | | | GGAGACCACCAAACCCTCCAAACA | |
| | | | AAGCAACAACAAGTATGCGGCCAG | |
| | | | CAGCTACCTGAGCCTGACGCCCGA | |
| | | | GCAGTGGAAGTCCC | |
| S376-1721 | CAGGTGCAGTTGGTGCAGTCTGGGA | 2748 | CAGTCTGTGCTGACGCAGCCGCCC | 2797 |
| | CTGAGGTGAGGGAGCCTGGGGCCTC | | TCAGTGTCTGGGGCCCCAGGGCAG | |
| | AGTGAAAGTCTCCTGCAAGGCTTCT | | AGGGTCACCATCTCCTGCACTGGG | |
| | GGATACACCTTCACCGGCTACTATG | | AGCAGCTCCAACATCGGGGCAGGT | |
| | TGCACTGGGTGCGGCAGGCCCCTGG | | TATGATGTACACTGGTACCAGCAG | |
| | ACAAGGACTTGAGTGGATGGGCTGG | | CTTCCAGGAACAGCCCCCAAACTC | |
| | GTCAACCCTGGCAGTGGTGACACAC | | CTCATCTATGGTAACAGCAATCGG | |
| | TCTATGCACAGAAGTTTCAGGGCAG | | CCCTCAGGGGTCCCTGACCGATTCT | |
| | GTTCACCTTGACCAGGGACATGTCC | | CTGGCTCCAAGTCTGGCACCTCAG | |
| | ATCACCACCGCCTACATGGAGCTGA | | CCTCCCTGGCCATCACTGGGCTCCA | |
| | GCAGCCTGAGATCTGACGACTCGGC | | GGCTGAGGATGAGGCTGATTATTA | |
| | CGTTTATTTCTGTTTCCGTGGATACA | | CTGCCAGTCCTATGACAGCAGCCT | |
| | GCTATGCAACCTTTGACTACTGGGG | | GAGTGGTTCTTTTTATGTCTTCGGA | |
| | CCAGGGAACCCTGGTCACCGTCTCC | | ACTGGGACCAAGGTCACCGTCCTA | |
| | TCAGCATCCCCGACCAGCCCCAAGG | | GGTCAGCCCAAGGCCAACCCCACT | |
| | TCTTCCCGCTGAGCCTCTGCAGCAC | | GTCACTCTGTTCCCGCCCTCCTCTG | |
| | CCAGCCAGATGGGAACGTGGTCATC | | AGGAGCTTCAAGCCAACAAGGCCA | |
| | GCCTGCCTGGTCCAGGGCTTCTTCC | | CACTGGTGTGTCTCATAAGTGACTT | |
| | CCCAGGAGCCACTCAGTGTGACCTG | | CTACCCGGGAGCCGTGACAGTGGC | |
| | GAGCGAAAGCGGACAGGGCGTGAC | | CTGGAAGGCAGATAGCAGCCCCGT | |
| | CGCCAGAAACTTCCC | | CAAGGCGGGAGTGGAGACCACCAC | |
| | | | ACCCTCCAAACAAAGCAACAACAA | |
| | | | GTACGCGGCCAGCAGCTA | |
| S376-2486 | CAGGTGCAGCTGGTGGAGTCTGGGG | 2749 | GAAATTGTGTTGACGCAGTCTCCA | 2798 |
| | GAGGCGTGGTCCAGCCTGGGAGGTC | | GGCACCCTGTCTTTGTCTCCAGGGG | |
| | CCTGAGACTCTCCTGTGCAGTCTCT | | AAAGAGCCACCCTCTCCTGCAGGG | |
| | GGATTCACCTTCAGTAGCTATGCTA | | CCAGTCAGAGTGTTAGCCGCAACT | |
| | TGCACTGGGTCCGCCAGGCTCCAGG | | ACTTAGCCTGGTACCAGCAGAAAC | |
| | CAAGGGGCTGGAGTGGGTGGCAGTT | | CTGGCCAGGCTCCCAGGCTCCTCAT | |
| | ATATCATATGATGGAAGCAATAAAT | | CTATAGTGCATCCAGCAGGGCCAC | |
| | ACTTCGCAGACTCCGTGAAGGGCCG | | TGGCATCCCAGACAGGTTCAGTGG | |
| | ATTCACCATCTCCAGAGACAATTCC | | CAGTGGGTCTGGGACAGACTTCAC | |
| | AAGAACACGCTGTATCTGCAAATGA | | TCTCACCATCAGCAGACTGGAGCC | |
| | ACAGCCTGAGAGCTGAGGACACGG | | TGAAGATTTTGCAGTGTATTACTGT | |
| | CTGTCTATTACTGTGCGAGAGGACG | | CAGCAGTATGGTGGCTCACTCACTT | |
| | TGGGAACTACTTTACCTACTTTGACT | | TCGGCGGAGGGACCAAGGTGGAGA | |
| | ACTGGGGCCAGGGAACCCTGGTCAC | | TCAAACGAACTGTGGCTGCACCAT | |
| | CGTCTCCTCAGCCTCCACCAAGGGC | | CTGTCTTCATCTTCCCGCCATCTGA | |
| | CCATCGGTCTTCCCCCTGGCACCCTC | | TGAGCAGTTGAAATCTGGAACTGC | |
| | CTCCAAGAGCACCTCTGGGGGCACA | | CTCTGTTGTGTGCCTGCTGAATAAC | |
| | GCGGCCCTGGGCTGCCTGGTCAAGG | | TTCTATCCCAGAGAGGCCAAAGTA | |
| | ACTACTTCCCCGAACCGGTGACGGT | | CAGTGGAAGGTGGATAACGC | |
| | GTCGTGGAACTCAGGCGCCCTGACC | | | |
| | AGCGGCGTGCACACCTTCCCGGCTG | | | |
| | TCCTACAGTCCTCAGG | | | |
| S376-780 | CAGGTGCAGCTGGTGGAGTCTGGGG | 2750 | GACATCCAGATGACCCAGTCTCCA | 2799 |
| | GAGGCGTGGTCCAGCCTGGGAGGTC | | TCCTCCCTGTCTGCATCTGTAGGAG | |
| | CCTGAGACTCTCCTGTGCAGCCTCT | | ACAGAGTCACCATCACTTGCCGGG | |
| | GGATTCACCTTCAGTAGCTATGGCA | | CGAGTCAGGGCATTAGCAATTATT | |
| | TGCACTGGGTCCGCCAGGCTCCAGG | | TAGCCTGGTATCAGCAGAAACCAG | |
| | CAAGGGGCTGGAGTGGGTGGCAGTT | | GGAAAGTTCCTAAGCTCCTGATCT | |
| | ATATCATATGATGGAAGTAATAAAT | | ATGCTGCATCCACTTTGCAATCAGG | |
| | ACTATGCAGACTCCGTGAAGGGCCG | | GGTCCCATCTCGGTTCAGTGGCAGT | |
| | ATTCACCATCTCCAGAGACAATTCC | | GGATCTGGGACAGATTTCACTCTC | |
| | AAGAACACGCTGTATCTGCAAATGA | | ACCATCAGCAGCCTGCAGCCTGAA | |
| | ACAGCCTGAGAGCTGAGGACACGG | | GATGTTGCAACTTATTACTGTCAAA | |
| | CTGTGTATTACTGTGCGAAAGAGGG | | AGTATAACAGTGCCCCTCGGACTTT | |
| | TGGGAGCTACTCCTACTACTACTAC | | CGGCCCTGGGACCAAAGTGGATAT | |
| | GGTATGGACGTCTGGGGCCAAGGG | | CAAACGAACTGTGGCTGCACCATC | |
| | ACCACGGTCACCGTCTCCTCAGGGA | | TGTCTTCATCTTCCCGCCATCTGAT | |
| | GTGCATCCGCCCCAACCCTTTTCCCC | | GAGCAGTTGAAATCTGGAACTGCC | |
| | CTCGTCTCCTGTGAGAATTCCCCGTC | | TCTGTTGTGTGCCTGCTGAATAACT | |
| | GGATACGAGCAGCGTG | | TCTATCCCAGAGAGGCCAAAGTAC | |
| | | | AGTGGAAGGTGGATAACGC | |
| S469-373 | GAGGTGCAGTTGGTGGAGTCTGGGG | 2751 | GAGGTGCAGTTGGTGGAGTCTGGG | 2800 |
| | GAGGGTTGGTCCAGCCTGGGGGGTC | | GGAGGGTTGGTCCAGCCTGGGGGG | |
| | CCTGAGACTCTCCTGTGTAGTCTCTG | | TCCCTGAGACTCTCCTGTGTAGTCT | |
| | GATTCACCTTTAGTAGGTATTGGAT | | CTGGATTCACCTTTAGTAGGTATTG | |
| | GAGCTGGGTCCGCCAGACTCCAGGG | | GATGAGCTGGGTCCGCCAGACTCC | |
| | AAGGGGCTGCAGTGGGTGGCTAAC | | AGGGAAGGGGCTGCAGTGGGTGGC | |
| | ATAAAGCAAGATGACACTAACAAA | | TAACATAAAGCAAGATGACACTAA | |
| | TTCTATGAAGACTCTGTGAAGGGCC | | CAAATTCTATGAAGACTCTGTGAA | |
| | GATTCACCACCTCCAGAGACAACGC | | GGGCCGATTCACCACCTCCAGAGA | |
| | CAAGAACTCACTATATCTGCAAATG | | CAACGCCAAGAACTCACTATATCT | |
| | AACAGCCTGAGAGCCGAGGACACG | | GCAAATGAACAGCCTGAGAGCCGA | |
| | GCCGTCTATTACTGTGCGAGAGGGG | | GGACACGGCCGTCTATTACTGTGC | |
| | GGGGCAGCTCGTCCGGGCTCTACTT | | GAGAGGGGGGGGCAGCTCGTCCGG | |
| | TGAGTCCTGGGGCCAGGGAACCCTG | | GCTCTACTTTGAGTCCTGGGGCCAG | |
| | GTCATCGTCTCCTCAGGGAGTGCAT | | GGAACCCTGGTCATCGTCTCCTCAG | |
| | CCGCCCCAACCCTTTTCCCCCTCGTC | | GGAGTGCATCCGCCCCAACCCTTTT | |
| | TCCTGTGAGAATTCCCCGTCGGATA | | CCCCCTCGTCTCCTGTGAGAATTCC | |
| | CGAGCAGCGTG | | CCGTCGGATACGAGCAGCGTG | |
| S48-144 | GAGGTGCAGCTGGTGGAGTCTGGGG | 2752 | GACATCCAGATGACCCAGTCTCCA | 2801 |
| | GAGACTTGGTACAGCCAGGGCGGTC | | TCCTCCCTGTCTGCATCTGTAGGAG | |
| | CCTGAGACTCTCCTGTACAGCTTCT | | ACAGAGTCACCATCACTTGCCGGG | |
| | GCATTCAACTTTGGTGATTATGCTAT | | CAAGCCAGAGCATTAGCACCTTTTT | |
| | GAGCTGGGTCCGCCAGGCTCCAGGG | | AAATTGGTATCAGCAGAAACCAGG | |
| | AAGGGGCTGGAGTGGGTAGGTTTTA | | GAAAGCCCCTAGTCTCCTGATCTAT | |
| | TTAGAAGTAAAGGTTATGGTGGGAC | | GCTGCATCCAGTTTGCAAAGTGGG | |
| | AACAGAATACGCCGCGTCTGTGAAA | | GTCCCATCAAGGTTCAGTGGCAGT | |
| | GGCAGATTCACCATCTCAAGAGATG | | GAATCTGGGACAGATTTCACTCTC | |
| | ATTCCAATCGCATCGCCTATCTGCA | | ACCATCAGCAGTCTGCAACCTGAA | |
| | AATGAACAGCCTGAAATCCGAGGA | | GATTTTGCAACTTACTACTGTCAAC | |
| | CACAGCCGTATATTACTGTAGTAGA | | AGAGTTACAGTACCCCACTCACTTT | |
| | GGGTACCAGCTGCCAAACTTATGGG | | CGGCGGAGGGACCAAGGTGGAGAT | |
| | GCCAGGGAACCCTGGTCACCGTCTC | | CAAACGAACTGTGGCTGCACCATC | |
| | CTCAGCATCCCCGACCAGCCCCAAG | | TGTCTTCATCTTCCCGCCATCTGAT | |
| | GTCTTCCCGCTGAGCCTCTGCAGCA | | GAGCAGTTGAAATCTGGAACTGCC | |
| | CCCAGCCAGATGGGAACGTGGTCAT | | TCTGTTGTGTGCCTGCTGAATAACT | |
| | CGCCTGCCTGGTCCAGGGCTTCTTC | | TCTATCCCAGAGAGGCCAAAGTAC | |
| | CCCCAGGAGCCACTCAGTGTGACCT | | AGTGGAAGGTGGATAACGC | |
| | GGAGCGAAAGCGGACAGGGCGTGA | | | |
| | CCGCCAGAAACTTCCC | | | |
| 128 | GAGGTGCACCTGGTGGAGTCTGGGG | 2753 | GAAATAGTGATGACGCAGTCTCCA | 2802 |
| | GAGGCTGGGTCCAGCCTGGGGGGTC | | GCCACCCTGTCTGTGTCTCCAGGGG | |
| | CCTGAGACTCTCCTGTGCAGCCTCT | | AAAGAGCCACCCTCTCCTGCAGGG | |
| | GGATTCACCTTGAGTACCTATTGGA | | CCAGTCAGAGTATCAGCAGCAAAT | |
| | TGAGCTGGGTCCGCCAGACTCCAGG | | TAGCCTGGTACCAGCAGAAACCTG | |
| | GGAGGGGCTGCAGTGGGTGGCCAA | | GCCAGGCTCCCAGGCTCCTCATCTA | |
| | CATAAAGCAAGATGGAAGTTCGAA | | TGGTGCGTCCACCAGGGCCACTGG | |
| | ATACTATGTGGACTCTGTGAAGGGC | | TATCCCAGCCAGGTTCAGTGGCAG | |
| | CGATTCACCATTTCCAGGGACAACG | | TGGGTCTGGGACAGAATTCACTCT | |
| | CCAAGAACTCAGTATATCTGCAAAT | | CACCATCAGCAGCATGCAGTCTGA | |
| | GAACAGCCTGAGAGGCGAGGACAC | | AGATTTTGCAGTTTATTACTGTCAG | |
| | GGCTGTGTATTATTGTGCGAGAGGG | | CAGTATAATTACTGGTACACTTTTG | |
| | GATGGGAGCAATTCCGGGATTTATT | | GCCAGGGGACCAAGCTGGAGATCA | |
| | TTGACTCCTGGGGCCAGGGAACCCT | | AACGAACTGTGGCTGCACCATCTG | |
| | GGTCACCGTCTCTTCAGCCTCCACC | | TCTTCATCTTCCCGCCATCTGATGA | |
| | AAGGGCCCATCGGTCTTCCCCCTGG | | GCAGTTGAAATCTGGAACTGCCTC | |
| | CGCCCTGCTCCAGGAGCACCTCCGA | | TGTTGTGTGCCTGCTGAATAACTTC | |
| | GAGCACAGCGGCCCTGGGCTGCCTG | | TATCCCAGAGAGGCCAAAGTACAG | |
| | GTCAAGGACTACTTCCCCGAACCGG | | TGGAAGGTGGATAACGCCCTCCAA | |
| | TGACGGTGTCGTGGAACTCAGGCGC | | TCGGGTAACTCCCAGGAGAGTGTC | |
| | TCTGACCAGCGGCGTGCACACCTTC | | ACAGAGCAGGACAGCAAGGACAG | |
| | CCGGCTGTCCTACAGTCCTCAGGA | | CACCTACAGCCTCAGCAGCACCCT | |
| | | | GACGCTGAGCAAAGCAGACTACGA | |
| | | | GAA | |
| S92-110 | GAGGTGCAGCTGGTGGAGTCTGGGG | 2754 | TCTTCTGAGCTGACTCAGGACCCTG | 2803 |
| | GAGGCTTGGTACAGCCTGGAGGGTC | | CTGTGTCTGTGGCCTTGGGACAGA | |
| | CCTGAGACTCTCCTGTGCAGCCTCT | | CAGTCAGGATCACATGCCAAGGAG | |
| | GGATTCACCTTCAGTAGTTATGAAA | | ACAGCCTCAGAAGCTATTATGCAA | |
| | TGAACTGGGTCCGCCAGGCTCCAGG | | GCTGGTACCAGCAGAAGCCAGGAC | |
| | GAAGGGGCTGGAGTGGGTTTCATAC | | AGGCCCCTGTACTTGTCATCTATGG | |
| | ATTAGTAGTAGTGGTAGTACCATAT | | TAAAAACAACCGGCCCTCAGGGAT | |
| | ACTACGCAGACTCTGTGAAGGGCCG | | CCCAGACCGATTCTCTGGCTCCAGC | |
| | ATTCACCATCTCCAGAGACAACGCC | | TCAGGAAACACAGCTTCCTTGACC | |
| | AAGAACTCACTGTATCTGCAAATGA | | ATCACTGGGGCTCAGGCGGAAGAT | |
| | ACAGCCTGAGAGCCGAGGACACGG | | GAGGCTGACTATTACTGTAACTCCC | |
| | CTGTTTATTACTGTGCGAGAGATAG | | GGGACAGCAGTGGTAACCGGGTGT | |
| | ACGTGGGGACTACGGCCGGTACTAC | | TCGGCGGAGGGACCAAGCTGACCG | |
| | TACGGTATGGACGTCTGGGGCCAAG | | TCCTAGGTCAGCCCAAGGCTGCCC | |
| | GGACCACGGTCACCGTCTCCTCAGG | | CCTCGGTCACTCTGTTCCCACCCTC | |
| | GAGTGCATCCGCCCCAACCCTTTTC | | CTCTGAGGAGCTTCAAGCCAACAA | |
| | CCCCTCGTCTCCTGTGAGAATTCCCC | | GGCCACACTGGTGTGTCTCATAAG | |
| | GTCGGATACGAGCAGCGTG | | TGACTTCTACCCGGGAGCCGTGAC | |
| | | | AGTGGCCTGGAAGGCAGATAGCAG | |
| | | | CCCCGTCAAGGCGGGAGTGGAGAC | |
| | | | CACCACACCCTCCAAACAAAGCAA | |
| | | | CAACAAGTACGCGGCCAGCAGCTA | |
| S92-2329 | GAGGTGCAGCTGGTGGAGTCTGGGG | 2755 | GAAATTGTGTTGACACAGTCTCCA | 2804 |
| | GAGGCCTGGTCAAGCCTGGGGGGTC | | GCTACCCTGTCTTTGTCTCCAGGGG | |
| | CCTGAGACTCTCCTGTGCAGCCTCT | | AAAGAGCCACCCTCTCCTGCAGGG | |
| | GGATTCACCTTCAGTAGCTATAGCA | | CCAGTCAGAGTGTTAGCAGCTACT | |
| | TGAACTGGGTCCGCCAGGCTCCAGG | | TAGCCTGGTACCAACAGAAACCTG | |
| | GAAGGGGCTGGAGTGGGTCTCATCC | | GCCAGGCTCCCAGGCTCCTCATCTA | |
| | ATTAGTAGTAGTGGTACTTACATAT | | TGATGCATTCAACAGGGCCACTGG | |
| | ACTACGCAGACTCAGTGAAGGGCCG | | CATCCCAGCCAGGTTCAGTGGCAG | |
| | ATTCACCATCTCCAGAGACAACGCC | | TGGGTCTGGGACAGACTTCACTCTC | |
| | AAGAACTCACTGTATCTGCAAATGA | | ACCATCAGCAGCCTAGAGCCTGAA | |
| | ACAGCCTGAGAGTCGAGGACACGG | | GATTTTGCAGTTTATTACTGTCAGC | |
| | CTGTGTATTACTGTGCCCAAAGTAT | | AGCGTAGCAACTGGCCTCGCACTT | |
| | TGCAGCTCGTCTCGACTGGTTCGAC | | TCGGCGGAGGGACCAAGGTGGAGA | |
| | CCCTGGGGCCAGGGAACCCTGGTCA | | TCAAACGAACTGTGGCTGCACCAT | |
| | CCGTCTCCTCAGGGAGTGCATCCGC | | CTGTCTTCATCTTCCCGCCATCTGA | |
| | CCCAACCCTTTTCCCCCTCGTCTCCT | | TGAGCAGTTGAAATCTGGAACTGC | |
| | GTGAGAATTCCCCGTCGGATACGAG | | CTCTGTTGTGTGCCTGCTGAATAAC | |
| | CAGCGTG | | TTCTATCCCAGAGAGGCCAAAGTA | |
| | | | CAGTGGAAGGTGGATAACGC | |

TABLE 3
Summary of SEQ ID NOS.

| | HC | VH | HCDR1 | HCDR2 | HCDR3 | HFRW1 | HFRW2 | HFRW3 | HFRW4 |
|---|---|---|---|---|---|---|---|---|---|
| S20-15 | 1 | 2 | 3 | 4 | 5 | 6 | 7 | 8 | 9 |
| S20-22 | 19 | 20 | 21 | 22 | 23 | 24 | 25 | 26 | 27 |
| S20-31 | 37 | 38 | 39 | 40 | 41 | 42 | 43 | 44 | 45 |
| S20-40 | 55 | 56 | 57 | 58 | 59 | 60 | 61 | 62 | 63 |
| S20-58 | 73 | 74 | 75 | 76 | 77 | 78 | 79 | 80 | 81 |
| S20-74 | 91 | 92 | 93 | 94 | 95 | 96 | 97 | 98 | 99 |
| S20-86 | 109 | 110 | 111 | 112 | 113 | 114 | 115 | 116 | 117 |
| S24-68 | 127 | 128 | 129 | 130 | 131 | 132 | 133 | 134 | 135 |
| S24-105 | 145 | 146 | 147 | 148 | 149 | 150 | 151 | 152 | 153 |
| S24-178 | 163 | 164 | 165 | 166 | 167 | 168 | 169 | 170 | 171 |
| S24-188 | 181 | 182 | 183 | 184 | 185 | 186 | 187 | 188 | 189 |
| S24-202 | 199 | 200 | 201 | 202 | 203 | 204 | 205 | 206 | 207 |
| S24-278 | 217 | 218 | 219 | 220 | 221 | 222 | 223 | 224 | 225 |
| S24-339 | 235 | 236 | 237 | 238 | 239 | 240 | 241 | 242 | 243 |
| S24-472 | 253 | 254 | 255 | 256 | 257 | 258 | 259 | 260 | 261 |
| S24-490 | 271 | 272 | 273 | 274 | 275 | 276 | 277 | 278 | 279 |
| S24-494 | 289 | 290 | 291 | 292 | 293 | 294 | 295 | 296 | 297 |
| S24-566 | 307 | 308 | 309 | 310 | 311 | 312 | 313 | 314 | 315 |
| S24-636 | 325 | 326 | 327 | 328 | 329 | 330 | 331 | 332 | 333 |
| S24-740 | 343 | 344 | 345 | 346 | 347 | 348 | 349 | 350 | 351 |
| S24-791 | 361 | 362 | 363 | 364 | 365 | 366 | 367 | 368 | 369 |
| S24-902 | 379 | 380 | 381 | 382 | 383 | 384 | 385 | 386 | 387 |
| S24-921 | 397 | 398 | 399 | 400 | 401 | 402 | 403 | 404 | 405 |
| S24-1063 | 415 | 416 | 417 | 418 | 419 | 420 | 421 | 422 | 423 |
| S24-1224 | 433 | 434 | 435 | 436 | 437 | 438 | 439 | 440 | 441 |
| S24-1271 | 451 | 452 | 453 | 454 | 455 | 456 | 457 | 458 | 459 |
| S24-1339 | 469 | 470 | 471 | 472 | 473 | 474 | 475 | 476 | 477 |
| S24-1345 | 487 | 488 | 489 | 490 | 491 | 492 | 493 | 494 | 495 |
| S24-1378 | 505 | 506 | 507 | 508 | 509 | 510 | 511 | 512 | 513 |
| S24-1379 | 523 | 524 | 525 | 526 | 527 | 528 | 529 | 530 | 531 |
| S24-1384 | 541 | 542 | 543 | 544 | 545 | 546 | 547 | 548 | 549 |
| S24-1476 | 559 | 560 | 561 | 562 | 563 | 564 | 565 | 566 | 567 |
| S24-1564 | 577 | 578 | 579 | 580 | 581 | 582 | 583 | 584 | 585 |
| S24-1636 | 595 | 596 | 597 | 598 | 599 | 600 | 601 | 602 | 603 |
| S24-1002 | 613 | 614 | 615 | 616 | 617 | 618 | 619 | 620 | 621 |
| S24-1301 | 631 | 632 | 633 | 634 | 635 | 636 | 637 | 638 | 639 |
| S24-223 | 649 | 650 | 651 | 652 | 653 | 654 | 655 | 656 | 657 |
| S24-461 | 667 | 668 | 669 | 670 | 671 | 672 | 673 | 674 | 675 |
| S24-511 | 685 | 686 | 687 | 688 | 689 | 690 | 691 | 692 | 693 |
| S24-788 | 703 | 704 | 705 | 706 | 707 | 708 | 709 | 710 | 711 |
| S24-821 | 721 | 722 | 723 | 724 | 725 | 726 | 727 | 728 | 729 |
| S144-67 | 739 | 740 | 741 | 742 | 743 | 744 | 745 | 746 | 747 |
| S144-69 | 757 | 758 | 759 | 760 | 761 | 762 | 763 | 764 | 765 |
| S144-94 | 775 | 776 | 777 | 778 | 779 | 780 | 781 | 782 | 783 |
| S144-113 | 793 | 794 | 795 | 796 | 797 | 798 | 799 | 800 | 801 |
| S144-175 | 811 | 812 | 813 | 814 | 815 | 816 | 817 | 818 | 819 |
| S144-208 | 829 | 830 | 831 | 832 | 833 | 834 | 835 | 836 | 837 |
| S144-339 | 847 | 848 | 849 | 850 | 851 | 852 | 853 | 854 | 855 |
| S144-359 | 865 | 866 | 867 | 868 | 869 | 870 | 871 | 872 | 873 |
| S144-460 | 883 | 884 | 885 | 886 | 887 | 888 | 889 | 890 | 891 |
| S144-466 | 901 | 902 | 903 | 904 | 905 | 906 | 907 | 908 | 909 |
| S144-469 | 919 | 920 | 921 | 922 | 923 | 924 | 925 | 926 | 927 |
| S144-509 | 937 | 938 | 939 | 940 | 941 | 942 | 943 | 944 | 945 |
| S144-516 | 955 | 956 | 957 | 958 | 959 | 960 | 961 | 962 | 963 |
| S144-568 | 973 | 974 | 975 | 976 | 977 | 978 | 979 | 980 | 981 |
| S144-576 | 991 | 992 | 993 | 994 | 995 | 996 | 997 | 998 | 999 |
| S144-588 | 1009 | 1010 | 1011 | 1012 | 1013 | 1014 | 1015 | 1016 | 1017 |
| S144-628 | 1027 | 1028 | 1029 | 1030 | 1031 | 1032 | 1033 | 1034 | 1035 |
| S144-740 | 1045 | 1046 | 1047 | 1048 | 1049 | 1050 | 1051 | 1052 | 1053 |
| S144-741 | 1063 | 1064 | 1065 | 1066 | 1067 | 1068 | 1069 | 1070 | 1071 |
| S144-803 | 1081 | 1082 | 1083 | 1084 | 1085 | 1086 | 1087 | 1088 | 1089 |
| S144-843 | 1099 | 1100 | 1101 | 1102 | 1103 | 1104 | 1105 | 1106 | 1107 |
| S144-877 | 1117 | 1118 | 1119 | 1120 | 1121 | 1122 | 1123 | 1124 | 1125 |
| S144-952 | 1135 | 1136 | 1137 | 1138 | 1139 | 1140 | 1141 | 1142 | 1143 |
| S144-971 | 1153 | 1154 | 1155 | 1156 | 1157 | 1158 | 1159 | 1160 | 1161 |
| S144-1036 | 1171 | 1172 | 1173 | 1174 | 1175 | 1176 | 1177 | 1178 | 1179 |
| S144-1079 | 1189 | 1190 | 1191 | 1192 | 1193 | 1194 | 1195 | 1196 | 1197 |
| S144-1299 | 1207 | 1208 | 1209 | 1210 | 1211 | 1212 | 1213 | 1214 | 1215 |
| S144-1339 | 1225 | 1226 | 1227 | 1228 | 1229 | 1230 | 1231 | 1232 | 1233 |
| S144-1406 | 1243 | 1244 | 1245 | 1246 | 1247 | 1248 | 1249 | 1250 | 1251 |
| S144-1407 | 1261 | 1262 | 1263 | 1264 | 1265 | 1266 | 1267 | 1268 | 1269 |
| S144-1569 | 1279 | 1280 | 1281 | 1282 | 1283 | 1284 | 1285 | 1286 | 1287 |
| S144-1641 | 1297 | 1298 | 1299 | 1300 | 1301 | 1302 | 1303 | 1304 | 1305 |
| S144-1827 | 1315 | 1316 | 1317 | 1318 | 1319 | 1320 | 1321 | 1322 | 1323 |
| S144-1848 | 1333 | 1334 | 1335 | 1336 | 1337 | 1338 | 1339 | 1340 | 1341 |
| S144-1850 | 1351 | 1352 | 1353 | 1354 | 1355 | 1356 | 1357 | 1358 | 1359 |
| S144-2234 | 1369 | 1370 | 1371 | 1372 | 1373 | 1374 | 1375 | 1376 | 1377 |
| S564-105 | 1387 | 1388 | 1389 | 1390 | 1391 | 1392 | 1393 | 1394 | 1395 |
| S564-14 | 1405 | 1406 | 1407 | 1408 | 1409 | 1410 | 1411 | 1412 | 1413 |
| S564-68 | 1423 | 1424 | 1425 | 1426 | 1427 | 1428 | 1429 | 1430 | 1431 |
| S564-98 | 1441 | 1442 | 1443 | 1444 | 1445 | 1446 | 1447 | 1448 | 1449 |
| S564-105 | 1459 | 1460 | 1461 | 1462 | 1463 | 1464 | 1465 | 1466 | 1467 |
| S564-134 | 1477 | 1478 | 1479 | 1480 | 1481 | 1482 | 1483 | 1484 | 1485 |
| S564-138 | 1495 | 1496 | 1497 | 1498 | 1499 | 1500 | 1501 | 1502 | 1503 |
| S564-152 | 1513 | 1514 | 1515 | 1516 | 1517 | 1518 | 1519 | 1520 | 1521 |
| S564-218 | 1531 | 1532 | 1533 | 1534 | 1535 | 1536 | 1537 | 1538 | 1539 |
| S564-249 | 1549 | 1550 | 1551 | 1552 | 1553 | 1554 | 1555 | 1556 | 1557 |
| S564-265 | 1567 | 1568 | 1569 | 1570 | 1571 | 1572 | 1573 | 1574 | 1575 |
| S564-275 | 1585 | 1586 | 1587 | 1588 | 1589 | 1590 | 1591 | 1592 | 1593 |
| S564-287 | 1603 | 1604 | 1605 | 1606 | 1607 | 1608 | 1609 | 1610 | 1611 |
| S116-2822 | 1825 | 1826 | 1827 | 1828 | 1829 | 1830 | 1831 | 1832 | 1833 |
| S116-2825 | 1843 | 1844 | 1845 | 1846 | 1847 | 1848 | 1849 | 1850 | 1851 |
| S116-3179 | 1861 | 1862 | 1863 | 1864 | 1865 | 1866 | 1867 | 1868 | 1869 |
| S144-121 | 1879 | 1880 | 1881 | 1882 | 1883 | 1884 | 1885 | 1886 | 1887 |
| S144-1364 | 1897 | 1898 | 1899 | 1900 | 1901 | 1902 | 1903 | 1904 | 1905 |
| S144-292 | 1915 | 1916 | 1917 | 1918 | 1919 | 1920 | 1921 | 1922 | 1923 |
| S155-37 | 1933 | 1934 | 1935 | 1936 | 1937 | 1938 | 1939 | 1940 | 1941 |
| S166-1318 | 1951 | 1952 | 1953 | 1954 | 1955 | 1956 | 1957 | 1958 | 1959 |
| S166-1366 | 1969 | 1970 | 1971 | 1972 | 1973 | 1974 | 1975 | 1976 | 1977 |
| S166-2395 | 1987 | 1988 | 1989 | 1990 | 1991 | 1992 | 1993 | 1994 | 1995 |
| S166-2620 | 2005 | 2006 | 2007 | 2008 | 2009 | 2010 | 2011 | 2012 | 2013 |
| S166-32 | 2023 | 2024 | 2025 | 2026 | 2027 | 2028 | 2029 | 2030 | 2031 |
| S171-1150 | 2041 | 2042 | 2043 | 2044 | 2045 | 2046 | 2047 | 2048 | 2049 |
| S171-1285 | 2059 | 2060 | 2061 | 2062 | 2063 | 2064 | 2065 | 2066 | 2067 |
| S171-692 | 2077 | 2078 | 2079 | 2080 | 2081 | 2082 | 2083 | 2084 | 2085 |
| S179-122 | 2095 | 2096 | 2097 | 2098 | 2099 | 2100 | 2101 | 2102 | 2103 |
| S179-20 | 2113 | 2114 | 2115 | 2116 | 2117 | 2118 | 2119 | 2120 | 2121 |
| S179-27 | 2131 | 2132 | 2133 | 2134 | 2135 | 2136 | 2137 | 2138 | 2139 |
| S179-28 | 2149 | 2150 | 2151 | 2152 | 2153 | 2154 | 2155 | 2156 | 2157 |
| S210-1139 | 2167 | 2168 | 2169 | 2170 | 2171 | 2172 | 2173 | 2174 | 2175 |
| S210-1262 | 2185 | 2186 | 2187 | 2188 | 2189 | 2190 | 2191 | 2192 | 2193 |
| S210-1611 | 2203 | 2204 | 2205 | 2206 | 2207 | 2208 | 2209 | 2210 | 2211 |
| S210-727 | 2221 | 2222 | 2223 | 2224 | 2225 | 2226 | 2227 | 2228 | 2229 |
| S210-852 | 2239 | 2240 | 2241 | 2242 | 2243 | 2244 | 2245 | 2246 | 2247 |
| S210-896 | 2257 | 2258 | 2259 | 2260 | 2261 | 2262 | 2263 | 2264 | 2265 |
| S2141-113 | 2275 | 2276 | 2277 | 2278 | 2279 | 2280 | 2281 | 2282 | 2283 |
| S2141-126 | 2293 | 2294 | 2295 | 2296 | 2297 | 2298 | 2299 | 2300 | 2301 |
| S2141-16 | 2311 | 2312 | 2313 | 2314 | 2315 | 2316 | 2317 | 2318 | 2319 |
| S2141-62 | 2329 | 2330 | 2331 | 2332 | 2333 | 2334 | 2335 | 2336 | 2337 |
| S2141-63 | 2347 | 2348 | 2349 | 2350 | 2351 | 2352 | 2353 | 2354 | 2355 |
| S2141-65 | 2365 | 2366 | 2367 | 2368 | 2369 | 2370 | 2371 | 2372 | 2373 |
| S2141-97 | 2383 | 2384 | 2385 | 2386 | 2387 | 2388 | 2389 | 2390 | 2391 |
| S24_342 | 2401 | 2402 | 2403 | 2404 | 2405 | 2406 | 2407 | 2408 | 2409 |
| S24-1047 | 2419 | 2420 | 2421 | 2422 | 2423 | 2424 | 2425 | 2426 | 2427 |
| S24-223 | 2437 | 2438 | 2439 | 2440 | 2441 | 2442 | 2443 | 2444 | 2445 |
| S24-237 | 2455 | 2456 | 2457 | 2458 | 2459 | 2460 | 246 | 2462 | 2463 |
| S305-1456 | 2473 | 2474 | 2475 | 2476 | 2477 | 2478 | 2479 | 2480 | 2481 |
| S305-223 | 2491 | 2492 | 2493 | 2494 | 2495 | 2496 | 2497 | 2498 | 2499 |
| S305-399 | 2509 | 2510 | 2511 | 2512 | 2513 | 2514 | 2515 | 2516 | 2517 |
| S305-968 | 2527 | 2528 | 2529 | 2530 | 2531 | 2532 | 2533 | 2534 | 2535 |
| S376-1070 | 2545 | 2546 | 2547 | 2548 | 2549 | 2550 | 2551 | 2552 | 2553 |
| S376-1721 | 2563 | 2564 | 2565 | 2566 | 2567 | 2568 | 2569 | 2570 | 2571 |
| S376-2486 | 2581 | 2582 | 2583 | 2584 | 2585 | 2586 | 2587 | 2588 | 2589 |
| S376-780 | 2599 | 2600 | 2601 | 2602 | 2603 | 2604 | 2605 | 2606 | 2607 |
| S469-373 | 2617 | 2618 | 2619 | 2620 | 2621 | 2622 | 2623 | 2624 | 2625 |
| S48-144 | 2635 | 2636 | 2637 | 2638 | 2639 | 2640 | 2641 | 2642 | 2643 |
| S564-128 | 2653 | 2654 | 2655 | 2656 | 2657 | 2658 | 2659 | 2660 | 2661 |
| S92-110 | 2671 | 2672 | 2673 | 2674 | 2675 | 2676 | 2677 | 2678 | 2679 |
| S92-2329 | 2689 | 2690 | 2691 | 2692 | 2693 | 2694 | 2695 | 2696 | 2697 |
| | LC | VL | LCDR1 | LCDR2 | LCDR3 | LFRW1 | LFRW2 | LFRW3 | LFRW4 |
| S20-15 | 10 | 11 | 12 | 13 | 14 | 15 | 16 | 17 | 18 |
| S20-22 | 28 | 29 | 30 | 31 | 32 | 33 | 34 | 35 | 36 |
| S20-31 | 46 | 47 | 48 | 49 | 50 | 51 | 52 | 53 | 54 |
| S20-40 | 64 | 65 | 66 | 67 | 68 | 69 | 70 | 71 | 72 |
| S20-58 | 82 | 83 | 84 | 85 | 86 | 87 | 88 | 89 | 90 |
| S20-74 | 100 | 101 | 102 | 103 | 104 | 105 | 106 | 107 | 108 |
| S20-86 | 118 | 119 | 120 | 121 | 122 | 123 | 124 | 125 | 126 |
| S24-68 | 136 | 137 | 138 | 139 | 140 | 141 | 142 | 143 | 144 |
| S24-105 | 154 | 155 | 156 | 157 | 158 | 159 | 160 | 161 | 162 |
| S24-178 | 172 | 173 | 174 | 175 | 176 | 177 | 178 | 179 | 180 |
| S24-188 | 190 | 191 | 192 | 193 | 194 | 195 | 196 | 197 | 198 |
| S24-202 | 208 | 209 | 210 | 211 | 212 | 213 | 214 | 215 | 216 |
| S24-278 | 226 | 227 | 228 | 229 | 230 | 231 | 232 | 233 | 234 |
| S24-339 | 244 | 245 | 246 | 247 | 248 | 249 | 250 | 251 | 252 |
| S24-472 | 262 | 263 | 264 | 265 | 266 | 267 | 268 | 269 | 270 |
| S24-490 | 280 | 281 | 282 | 283 | 284 | 285 | 286 | 287 | 288 |
| S24-494 | 298 | 299 | 300 | 301 | 302 | 303 | 304 | 305 | 306 |
| S24-566 | 316 | 317 | 318 | 319 | 320 | 321 | 322 | 323 | 324 |
| S24-636 | 334 | 335 | 336 | 337 | 338 | 339 | 340 | 341 | 342 |
| S24-740 | 352 | 353 | 354 | 355 | 356 | 357 | 358 | 359 | 360 |
| S24-791 | 370 | 371 | 372 | 373 | 374 | 375 | 376 | 377 | 378 |
| S24-902 | 388 | 389 | 390 | 391 | 392 | 393 | 394 | 395 | 396 |
| S24-921 | 406 | 407 | 408 | 409 | 410 | 411 | 412 | 413 | 414 |
| S24-1063 | 424 | 425 | 426 | 427 | 428 | 429 | 430 | 431 | 432 |
| S24-1224 | 442 | 443 | 444 | 445 | 446 | 447 | 448 | 449 | 450 |
| S24-1271 | 460 | 461 | 462 | 463 | 464 | 465 | 466 | 467 | 468 |
| S24-1339 | 478 | 479 | 480 | 481 | 482 | 483 | 484 | 485 | 486 |
| S24-1345 | 496 | 497 | 498 | 499 | 500 | 501 | 502 | 503 | 504 |
| S24-1378 | 514 | 515 | 516 | 517 | 518 | 519 | 520 | 521 | 522 |
| S24-1379 | 532 | 533 | 534 | 535 | 536 | 537 | 538 | 539 | 540 |
| S24-1384 | 550 | 551 | 552 | 553 | 554 | 555 | 556 | 557 | 558 |
| S24-1476 | 568 | 569 | 570 | 571 | 572 | 573 | 574 | 575 | 576 |
| S24-1564 | 586 | 587 | 588 | 589 | 590 | 591 | 592 | 593 | 594 |
| S24-1636 | 604 | 605 | 606 | 607 | 608 | 609 | 610 | 611 | 612 |
| S24-1002 | 622 | 623 | 624 | 625 | 626 | 627 | 628 | 629 | 630 |
| S24-1301 | 640 | 641 | 642 | 643 | 644 | 645 | 646 | 647 | 648 |
| S24-223 | 658 | 659 | 660 | 661 | 662 | 663 | 664 | 665 | 666 |
| S24-461 | 676 | 677 | 678 | 679 | 680 | 681 | 682 | 683 | 684 |
| S24-511 | 694 | 695 | 696 | 697 | 698 | 699 | 700 | 701 | 702 |
| S24-788 | 712 | 713 | 714 | 715 | 716 | 717 | 718 | 719 | 720 |
| S24-821 | 730 | 731 | 732 | 733 | 734 | 735 | 736 | 737 | 738 |
| S144-67 | 748 | 749 | 750 | 751 | 752 | 753 | 754 | 755 | 756 |
| S144-69 | 766 | 767 | 768 | 769 | 770 | 771 | 772 | 773 | 774 |
| S144-94 | 784 | 785 | 786 | 787 | 788 | 789 | 790 | 791 | 792 |
| S144-113 | 802 | 803 | 804 | 805 | 806 | 807 | 808 | 809 | 810 |
| S144-175 | 820 | 821 | 822 | 823 | 824 | 825 | 826 | 827 | 828 |
| S144-208 | 838 | 839 | 840 | 841 | 842 | 843 | 844 | 845 | 846 |
| S144-339 | 856 | 857 | 858 | 859 | 860 | 861 | 862 | 863 | 864 |
| S144-359 | 874 | 875 | 876 | 877 | 878 | 879 | 880 | 881 | 882 |
| S144-460 | 892 | 893 | 894 | 895 | 896 | 897 | 898 | 899 | 900 |
| S144-466 | 910 | 911 | 912 | 913 | 914 | 915 | 916 | 917 | 918 |
| S144-469 | 928 | 929 | 930 | 931 | 932 | 933 | 934 | 935 | 936 |
| S144-509 | 946 | 947 | 948 | 949 | 950 | 951 | 952 | 953 | 954 |
| S144-516 | 964 | 965 | 966 | 967 | 968 | 969 | 970 | 971 | 972 |
| S144-568 | 982 | 983 | 984 | 985 | 986 | 987 | 988 | 989 | 990 |
| S144-576 | 1000 | 1001 | 1002 | 1003 | 1004 | 1005 | 1006 | 1007 | 1008 |
| S144-588 | 1018 | 1019 | 1020 | 1021 | 1022 | 1023 | 1024 | 1025 | 1026 |
| S144-628 | 1036 | 1037 | 1038 | 1039 | 1040 | 1041 | 1042 | 1043 | 1044 |
| S144-740 | 1054 | 1055 | 1056 | 1057 | 1058 | 1059 | 1060 | 1061 | 1062 |
| S144-741 | 1072 | 1073 | 1074 | 1075 | 1076 | 1077 | 1078 | 1079 | 1080 |
| S144-803 | 1090 | 1091 | 1092 | 1093 | 1094 | 1095 | 1096 | 1097 | 1098 |
| S144-843 | 1108 | 1109 | 1110 | 1111 | 1112 | 1113 | 1114 | 1115 | 1116 |
| S144-877 | 1126 | 1127 | 1128 | 1129 | 1130 | 1131 | 1132 | 1133 | 1134 |
| S144-952 | 1144 | 1145 | 1146 | 1147 | 1148 | 1149 | 1150 | 1151 | 1152 |
| S144-971 | 1162 | 1163 | 1164 | 1165 | 1166 | 1167 | 1168 | 1169 | 1170 |
| S144-1036 | 1180 | 1181 | 1182 | 1183 | 1184 | 1185 | 1186 | 1187 | 1188 |
| S144-1079 | 1198 | 1199 | 1200 | 1201 | 1202 | 1203 | 1204 | 1205 | 1206 |
| S144-1299 | 1216 | 1217 | 1218 | 1219 | 1220 | 1221 | 1222 | 1223 | 1224 |
| S144-1339 | 1234 | 1235 | 1236 | 1237 | 1238 | 1239 | 1240 | 1241 | 1242 |
| S144-1406 | 1252 | 1253 | 1254 | 1255 | 1256 | 1257 | 1258 | 1259 | 1260 |
| S144-1407 | 1270 | 1271 | 1272 | 1273 | 1274 | 1275 | 1276 | 1277 | 1278 |
| S144-1569 | 1288 | 1289 | 1290 | 1291 | 1292 | 1293 | 1294 | 1295 | 1296 |
| S144-1641 | 1306 | 1307 | 1308 | 1309 | 1310 | 1311 | 1312 | 1313 | 1314 |
| S144-1827 | 1324 | 1325 | 1326 | 1327 | 1328 | 1329 | 1330 | 1331 | 1332 |
| S144-1848 | 1342 | 1343 | 1344 | 1345 | 1346 | 1347 | 1348 | 1349 | 1350 |
| S144-1850 | 1360 | 1361 | 1362 | 1363 | 1364 | 1365 | 1366 | 1367 | 1368 |
| S144-2234 | 1378 | 1379 | 1380 | 1381 | 1382 | 1383 | 1384 | 1385 | 1386 |
| S564-105 | 1396 | 1397 | 1398 | 1399 | 1400 | 1401 | 1402 | 1403 | 1404 |
| S564-14 | 1414 | 1415 | 1416 | 1417 | 1418 | 1419 | 1420 | 1421 | 1422 |
| S564-68 | 1432 | 1433 | 1434 | 1435 | 1436 | 1437 | 1438 | 1439 | 1440 |
| S564-98 | 1450 | 1451 | 1452 | 1453 | 1454 | 1455 | 1456 | 1457 | 1458 |
| S564-105 | 1468 | 1469 | 1470 | 1471 | 1472 | 1473 | 1474 | 1475 | 1476 |
| S564-134 | 1486 | 1487 | 1488 | 1489 | 1490 | 1491 | 1492 | 1493 | 1494 |
| S564-138 | 1504 | 1505 | 1506 | 1507 | 1508 | 1509 | 1510 | 1511 | 1512 |
| S564-152 | 1522 | 1523 | 1524 | 1525 | 1526 | 1527 | 1528 | 1529 | 1530 |
| S564-218 | 1540 | 1541 | 1542 | 1543 | 1544 | 1545 | 1546 | 1547 | 1548 |
| S564-249 | 1558 | 1559 | 1560 | 1561 | 1562 | 1563 | 1564 | 1565 | 1566 |
| S564-265 | 1576 | 1577 | 1578 | 1579 | 1580 | 1581 | 1582 | 1583 | 1584 |
| S564-275 | 1594 | 1595 | 1596 | 1597 | 1598 | 1599 | 1600 | 1601 | 1602 |
| S564-287 | 1612 | 1613 | 1614 | 1615 | 1616 | 1617 | 1618 | 1619 | 1620 |
| S116-2822 | 1834 | 1835 | 1836 | 1837 | 1838 | 1839 | 1840 | 1841 | 1842 |
| S116-2825 | 1852 | 1853 | 1854 | 1855 | 1856 | 1857 | 1858 | 1859 | 1860 |
| S116-3179 | 1870 | 1871 | 187 | 1873 | 1874 | 1875 | 1876 | 1877 | 1878 |
| S144-121 | 1888 | 1889 | 1890 | 1891 | 1892 | 1893 | 1894 | 1895 | 1896 |
| S144-1364 | 1906 | 1907 | 1908 | 1909 | 1910 | 1911 | 1912 | 1913 | 1914 |
| S144-292 | 1924 | 1925 | 1926 | 1927 | 1928 | 1929 | 1930 | 1931 | 1932 |
| S155-37 | 1942 | 1943 | 1944 | 1945 | 1946 | 1947 | 1948 | 1949 | 1950 |
| S166-1318 | 1960 | 1961 | 1962 | 1963 | 1964 | 1965 | 1966 | 1967 | 1968 |
| S166-1366 | 1978 | 1979 | 1980 | 1981 | 1982 | 1983 | 1984 | 1985 | 1986 |
| S166-2395 | 1996 | 1997 | 1998 | 1999 | 2000 | 2001 | 2002 | 2003 | 2004 |
| S166-2620 | 2014 | 2015 | 2016 | 2017 | 2018 | 2019 | 2020 | 2021 | 2022 |
| S166-32 | 2032 | 2033 | 2034 | 2035 | 2036 | 2037 | 2038 | 2039 | 2040 |
| S171-1150 | 2050 | 2051 | 2052 | 2053 | 2054 | 2055 | 2056 | 2057 | 2058 |
| S171-1285 | 2068 | 2069 | 2070 | 2071 | 2072 | 2073 | 2074 | 2075 | 2076 |
| S171-692 | 2086 | 2087 | 2088 | 2089 | 2090 | 2091 | 2092 | 2093 | 2094 |
| S179-122 | 2104 | 2105 | 2106 | 2107 | 2108 | 2109 | 2110 | 2111 | 2112 |
| S179-20 | 2122 | 2123 | 2124 | 2125 | 2126 | 2127 | 2128 | 2129 | 2130 |
| S179-27 | 2140 | 2141 | 2142 | 2143 | 2144 | 2145 | 2146 | 2147 | 2148 |
| S179-28 | 2158 | 2159 | 2160 | 2161 | 2162 | 2163 | 2164 | 2165 | 2166 |
| S210-1139 | 2176 | 2177 | 2178 | 2179 | 2180 | 2181 | 2182 | 2183 | 2184 |
| S210-1262 | 2194 | 2195 | 2196 | 2197 | 2198 | 2199 | 2200 | 2201 | 2202 |
| S210-1611 | 2212 | 2213 | 2214 | 2215 | 2216 | 2217 | 2218 | 2219 | 2220 |
| S210-727 | 2230 | 2231 | 2232 | 2233 | 2234 | 2235 | 2236 | 2237 | 2238 |
| S210-852 | 2248 | 2249 | 2250 | 2251 | 2252 | 2253 | 2254 | 2255 | 2256 |
| S210-896 | 2266 | 2267 | 2268 | 2269 | 2270 | 2271 | 2272 | 2273 | 2274 |
| S2141-113 | 2284 | 2285 | 2286 | 2287 | 2288 | 2289 | 2290 | 2291 | 2292 |
| S2141-126 | 2302 | 2303 | 2304 | 2305 | 2306 | 2307 | 2308 | 2309 | 2310 |
| S2141-16 | 2320 | 2321 | 2322 | 2323 | 2324 | 2325 | 2326 | 2327 | 2328 |
| S2141-62 | 2338 | 2339 | 2340 | 2341 | 2342 | 2343 | 2344 | 2345 | 2346 |
| S2141-63 | 2356 | 2357 | 2358 | 2359 | 2360 | 2361 | 2362 | 2363 | 2364 |
| S2141-65 | 2374 | 2375 | 2376 | 2377 | 2378 | 2379 | 2380 | 2381 | 2382 |
| S2141-97 | 2392 | 2393 | 2394 | 2395 | 2396 | 2397 | 2398 | 2399 | 2400 |
| S24_342 | 2410 | 2411 | 2412 | 2413 | 2414 | 2415 | 2416 | 2417 | 2418 |
| S24-1047 | 2428 | 2429 | 2430 | 2431 | 2432 | 2433 | 2434 | 2435 | 2436 |
| S24-223 | 2446 | 2447 | 2448 | 2449 | 2450 | 2451 | 2452 | 2453 | 2454 |
| S24-237 | 2464 | 2465 | 2466 | 2467 | 2468 | 2469 | 2470 | 2471 | 2472 |
| S305-1456 | 2482 | 2483 | 2484 | 2485 | 2486 | 2487 | 2488 | 2489 | 2490 |
| S305-223 | 2500 | 2501 | 2502 | 2503 | 2504 | 2505 | 2506 | 2507 | 2508 |
| S305-399 | 2518 | 2519 | 2520 | 2521 | 2522 | 2523 | 2524 | 2525 | 2526 |
| S305-968 | 2536 | 2537 | 2538 | 2539 | 2540 | 2541 | 2542 | 2543 | 2544 |
| S376-1070 | 2554 | 2555 | 2556 | 2557 | 2558 | 2559 | 2560 | 2561 | 2562 |
| S376-1721 | 2572 | 2573 | 2574 | 2575 | 2576 | 2577 | 2578 | 2579 | 2580 |
| S376-2486 | 2590 | 2591 | 2592 | 2593 | 2594 | 2595 | 2596 | 2597 | 2598 |
| S376-780 | 2608 | 2609 | 2610 | 2611 | 2612 | 2613 | 2614 | 2615 | 2616 |
| S469-373 | 2626 | 2627 | 2628 | 2629 | 2630 | 2631 | 2632 | 2633 | 2634 |
| S48-144 | 2644 | 2645 | 2646 | 2647 | 2648 | 2649 | 2650 | 2651 | 2652 |
| S564-128 | 2662 | 2663 | 2664 | 2665 | 2666 | 2667 | 2668 | 2669 | 2670 |
| S92-110 | 2680 | 2681 | 2682 | 2683 | 2684 | 2685 | 2686 | 2687 | 2688 |
| S92-2329 | 2698 | 2699 | 2700 | 2701 | 2702 | 2703 | 2704 | 2705 | 2706 |

1. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

2. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within +2 are included, in other embodiments, those which are within ±1 are included, and in still other embodiments, those within are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### VII. Nucleic Acids

In certain embodiments, nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

B. Mutation

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing the engineered cells of the disclosure. In a preferred embodiment, the nucleic acid molecules are oligonucleotides.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### VIII. Polypeptide Expression

In some aspects, there are nucleic acid molecule encoding polypeptides, antibodies, or antigen binding fragments of the disclosure. The nucleic acid molecules may be used to express large quantities of polypeptides. If the nucleic acid molecules are derived from a non-human, non-transgenic animal, the nucleic acid molecules may be used for humanization of the antibody or TCR genes.

A. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody heavy and/or light chain, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

B. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### IX. Pharmaceutical Compositions

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., compositions comprising SARS-CoV-2 protein-binding polypeptides) are administered to a subject intravenously.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, subcutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 ng/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about μM to 50 μM; or about 25 μM to 150 μM; or about 25 μM to 100 μM; or about 25 μM to 50 μM, or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### X. Detectable Labels

In some aspects of this disclosure, it will be useful to detectably or therapeutically label a Fab polypeptide or protein G Fab-binding domain. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue.™., and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

### XI. Sample Preparation

In certain aspects, methods involve obtaining or evaluating a sample from a subject. The sample may include a sample obtained from any source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### XII. Host Cells

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In certain embodiments transfection can be carried out on any prokaryotic or eukaryotic cell. In some aspects electroporation involves transfection of a human cell. In other aspects electroporation involves transfection of an animal cell. In certain aspects transfection involves transfection of a cell line or a hybrid cell type. In some aspects the cell or cells being transfected are cancer cells, tumor cells or immortalized cells. In some instances tumor, cancer, immortalized cells or cell lines are induced and in other instances tumor, cancer, immortalized cells or cell lines enter their respective state or condition naturally. In certain aspects the cells or cell lines can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NS0, Panc-1, PC12, PC-3, Peripheral blood cells, Plasma cells, Primary Fibroblasts, RBL, Renca, RLE, SF21, SF9, SH-SYSY, SK-MES-1, SK-N-SH, SL3, SW403, Stimulus-triggered Acquisition of Pluripotency (STAP) cell or derivate SW403, T-cells, THP-1, Tumor cells, U2OS, U937, peripheral blood lymphocytes, expanded T cells, hematopoietic stem cells, or Vero cells.

### XIII. Kits

Certain aspects of the present invention also concern kits containing compositions of the disclosure or compositions to implement methods of the disclosure. In some embodiments, kits can be used to detect the presence of a SARS-CoV-2 virus in a sample. In certain embodiments, a kit contains, contains at least or contains at most 1, 2, 3, 4, 5, 6, 7, 8, 9, 11, 12, 13, 14, 15, 16, 17, 18, 19, 20, 21, 22, 23, 24, 25, 26, 27, 28, 29, 30, 31, 32, 33, 34, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, a kit contains one or more polypeptides capable of binding to a SARS-CoV-2 spike protein, including polypeptides disclosed herein. For example, a kit may comprise at least 1, 2, 3, 4, 5, 6, 7, 8, 9, 10 or more Fabs disclosed herein for detecting a SARS-CoV-2 spike protein. In some embodiments, a kit comprises a detection pair. In some embodiments, a kit comprises an enzyme. In some embodiments, a kit comprises a substrate for an enzyme.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. In certain aspects, negative and/or positive control nucleic acids, probes, and inhibitors are included in some kit embodiments.

Kits may further comprise instructions for use. For example, in some embodiments, a kit comprises instructions for detecting a SARS-CoV-2 virus in a sample.

It is contemplated that any method or composition described herein can be implemented with respect to any other method or composition described herein and that different embodiments may be combined. The claims originally filed are contemplated to cover claims that are multiply dependent on any filed claim or combination of filed claims.

### XIV. Examples

The following examples are included to demonstrate certain embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—Distinct B Cell Subsets Give Rise to Antigen-Specific Antibody Responses Against SARS-CoV-2

A. Results

1. SARS-CoV-2-Specific B Cell Sequencing

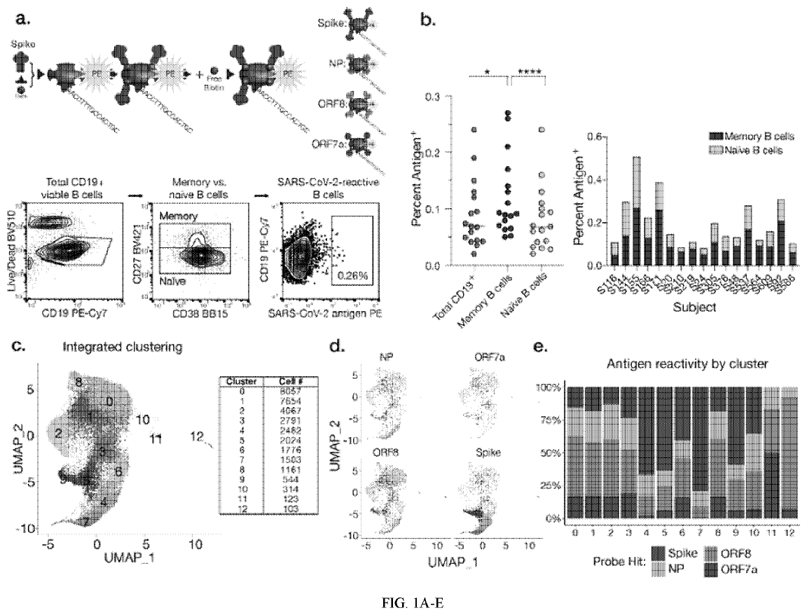

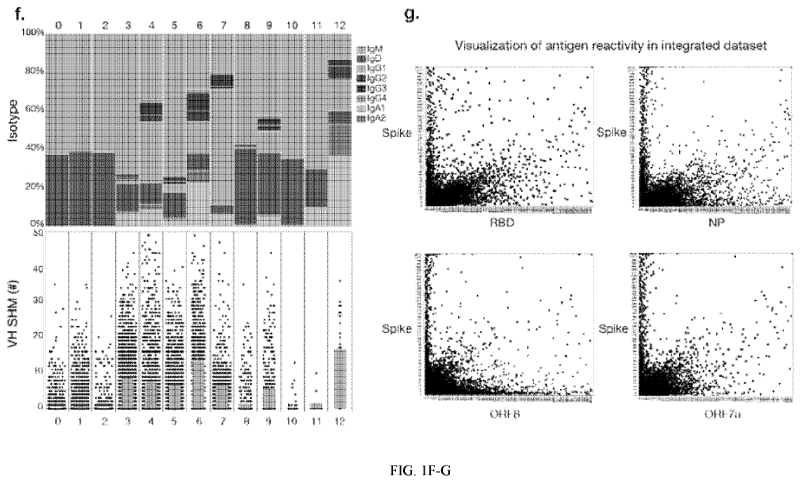

Serum antibodies and MBCs have potential to act as the first line of defense against SARS-CoV-2 infection^{11, 15-17}. To determine the landscape of antibody reactivity toward distinct SARS-CoV-2 viral targets, the inventors collected peripheral blood mononuclear cells (PBMCs) and serum from 25 subjects between April and May of 2020 upon recovery from SARS-CoV-2 viral infection (Extended Data Table 1 and Extended Data Table 2). To identify B cells specific to the SARS-CoV-2 spike protein, spike RBD, ORF7a, ORF8, and NP, the inventors generated probes to bait-sort enriched B cells for subsequent single cell RNA sequencing analysis by conjugating distinct phycoerythrin (PE)-streptavidin (SA)-oligos to individual biotinylated antigens (FIG. 1a).

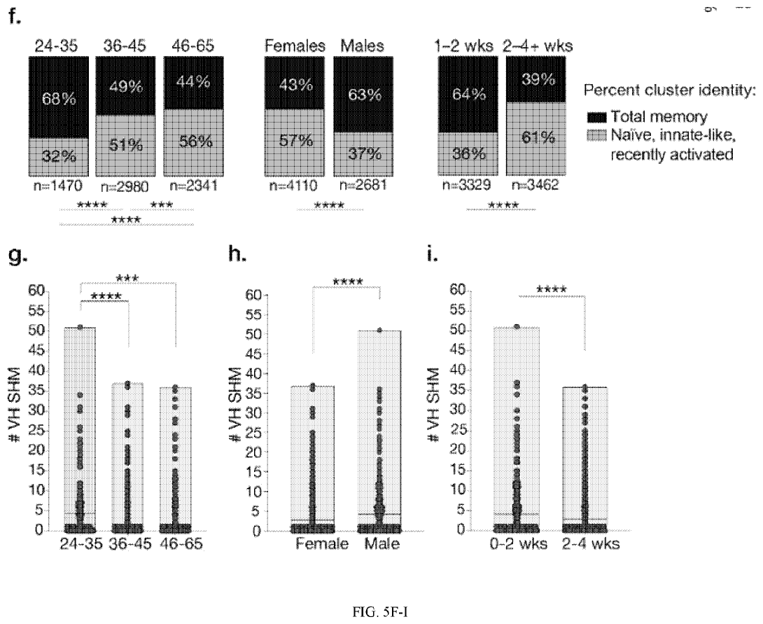

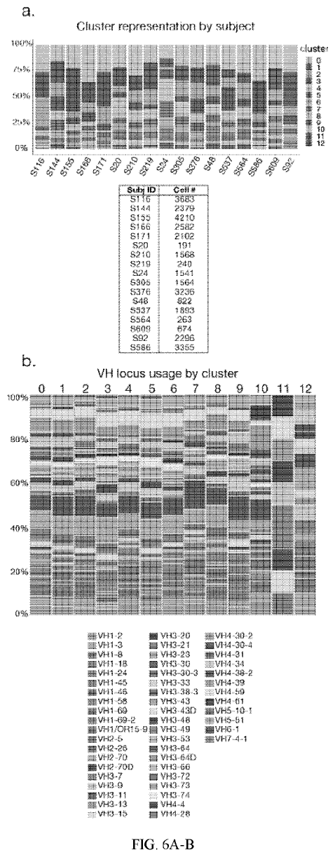

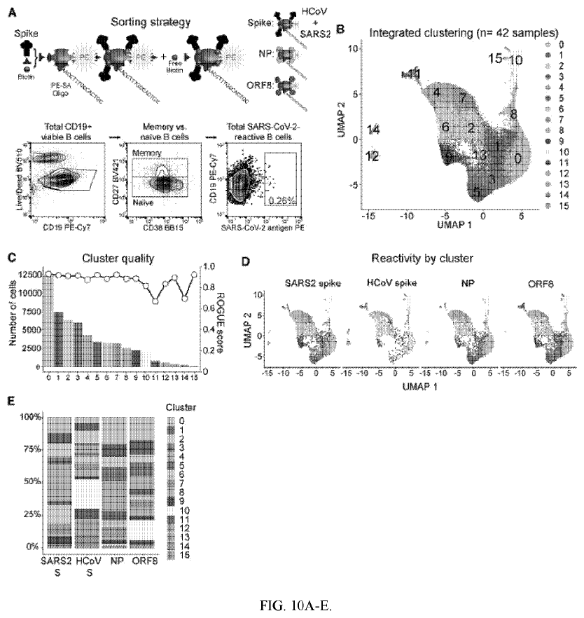

From 25 subjects analyzed, the inventors detected small percentages (0.02-0.26%) of SARS-CoV-2-reactive total CD19⁺ B cells, which were subsequently used to prepare 5′ transcriptome, immunoglobulin (Ig) VDJ, and antigen-specific probe feature libraries for sequencing (FIG. 1a, b). The inventors detected increased percentages of antigen-specific B cells within the memory B cell (MBC) compartment FIG. 1B, CD19⁺CD27⁺CD38^{int}), though the inventors sorted on total CD19⁺ antigen-specific B cells to ensure adequate coverage of all potential reactive B cells and to optimize sequence library preparation and downstream analysis as the antigen-specific population was rare. The inventors integrated data from 17 subjects with high-quality sequencing results using Seurat to remove batch effects and identified 12 transcriptionally distinct B cell clusters based on transcriptional expression profiles (FIG. 1c). It was immediately evident that B cells specific to the spike, NP, ORF7a, and ORF8 were found amongst multiple B cell subsets, with spike-specific B cells substantially enriched in clusters 4, 5, 7, and 9 (FIGS. 1d, e). Analysis of Ig isotypes and degree of Ig variable heavy chain somatic hypermutations (VH SHM) suggested that clusters 0-2, 8, 10, and 11 represented naïve- or innate-like B cell clusters predominantly composed of IgM and IgD B cells. In contrast, clusters 3, 4, 5, 6, 7, 9, and 12 strongly indicated B cell subsets more similar to MBCs or plasma cells, as they exhibited a higher degree of class switch recombination (CSR) and/or increased numbers of VH SHM (FIG. 10. The inventors detected variation in the percentage of total cells sorted per cluster amongst individual patients, reflecting differences in the biology of individual responses to SARS-CoV-2, as the inventors expand upon later (FIG. 6a). No major differences in VH gene usage across clusters were evident, though the inventors identified enrichment of VH1-24 in cluster 7, which the inventors later identify as exclusively utilized by spike-reactive B cells (FIG. 6b).

The inventors next addressed whether the probe intensities generated from the feature libraries correlated with antigen-specific reactivity by plotting intensities for distinct probes against one another to observe true specificity (cells that fall directly onto the x or y axis) vs. non-specific binding (cells that fall on the diagonal). The inventors observed hundreds of cells specific to the spike, ORF8, NP, and to a lesser degree, ORF7a (FIG. 1g). For clusters 1, 2, and 8, the inventors observed that the majority of cells were not uniquely specific for any one probe, and instead tended to bind more than one probe in a polyreactive or non-specific manner, consistent with innate-like B cells¹⁸. Finally, clusters 4, 5, 6, 7, and 9 exhibited highly specific binding toward the spike, NP, and ORF8, with the majority targeting the spike (FIG. 6c). Together, the data suggest the B cell response to SARS-CoV-2 is comprised of multiple functionally distinct B cell subsets enriched for binding to distinct viral targets.

2. SARS-CoV-2-Specific B Cell Subsets

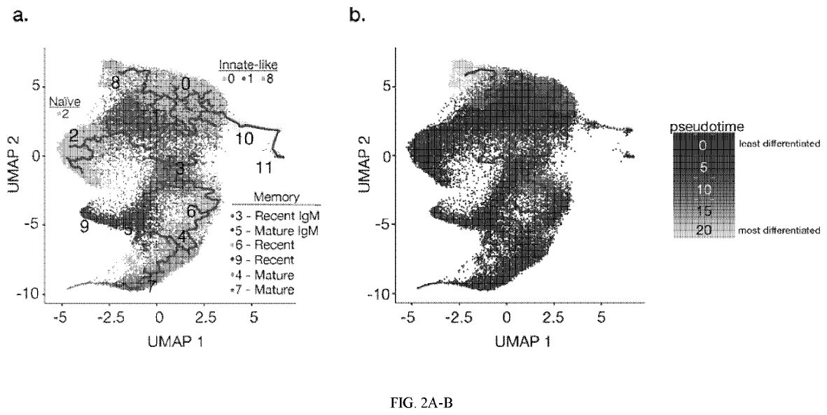

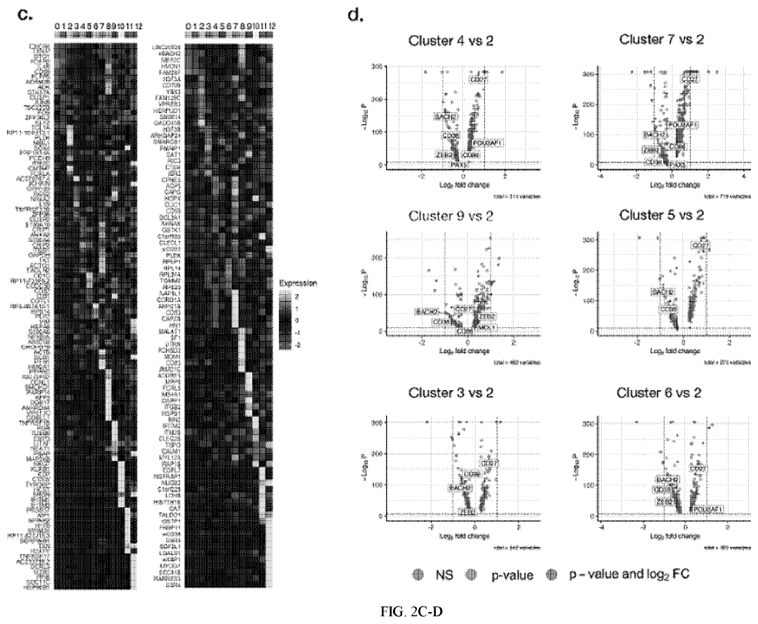

To discern the identities of distinct B cell subsets, the inventors further analyzed Ig repertoire, differentially expressed genes, and performed pseudotime analyses of integrated clusters. For pseudotime analysis, the inventors rooted the data on cluster 2, as cells within this cluster expressed Ig genes with little to no SHM or CSR (FIG. 1f) and displayed low probe reactivity (FIG. 6c), suggesting this subset is comprised of true naïve B cells. Pseudotime analysis rooted on cluster 2 identified clusters 0, 1, and 8 in various stages of differentiation, suggestive of recent activation (FIG. 2a-b). As they displayed little CSR or SHM (FIG. 10, the inventors therefore categorized these subsets as innate-like or possibly germinal center independent. Clusters 3 and 5 appeared to be specific IgM memory subsets (FIG. 1f, FIG. 6c), while clusters 4, 7, 9, and 12 displayed high specificity, CSR, and SHM, demonstrating an affinity-matured memory phenotype (FIG. 1f, FIG. 6c). As naïve B cells and MBCs are quiescent, clusters 4, 5, 7, and 9 were similar to cluster 2 in pseudotime analysis (FIG. 2a-b)¹⁹. Lastly, cluster 6 was of interest as these cells displayed the greatest frequency of SHM and IgA CSR, and may have arisen in the context of a mucosal immune response.

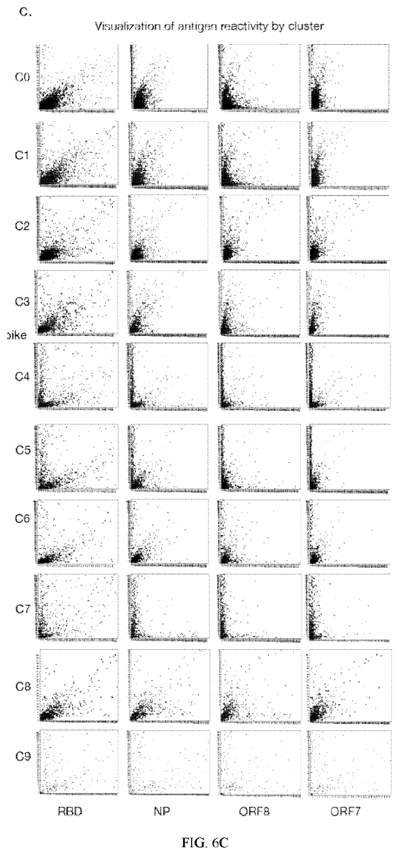

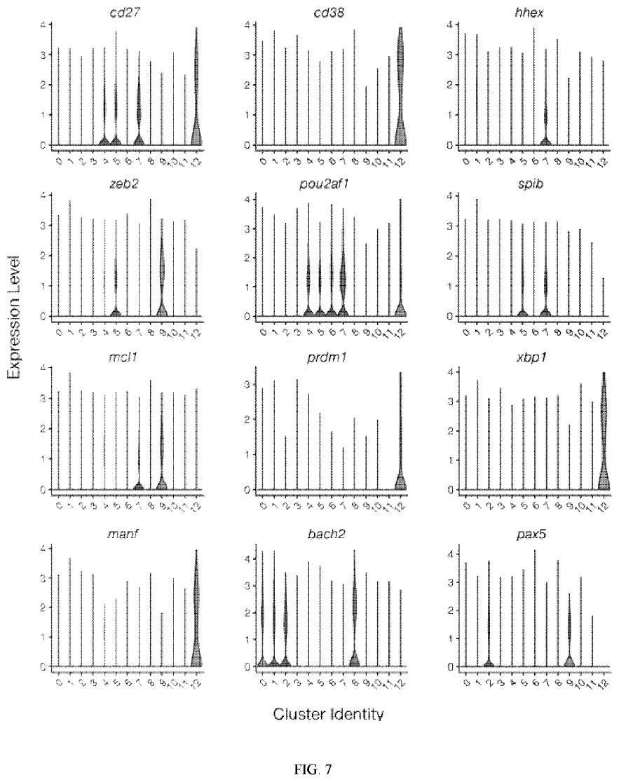

In-depth analysis of select genes including those related to B cell fate, MBC differentiation and maintenance, and long-lived plasma cells (LLPCs) helped to further reveal the identities of select clusters. Genes associated with MBCs (cd27, cd38, cd86, pon2af), repression of apoptosis (mcl1), early commitment to B cell fate (zeb2), repression of LLPC fate (spiB, pax5, bach2), and early B cell activation and proliferation (bach2) confirmed clusters 3, 4, 5, 7 and 9 as MBCs though with varying degrees of differentiation, CSR, and SHIM (FIG. 2b-c, FIG. 7). Notably, the inventors identified upregulation of the transcription factor hhex in cluster 7, which has recently been shown to be involved in MBC differentiation in mice (FIG. 7)²⁰. Lastly, cluster 12 appeared to be LLPCs or precursors thereof by expression of genes associated with LLPC fate, including prdm1, xbp1, and manf (FIG. 7)^{19,21,22}. Together with the antigen-specific probe data (FIG. 1), these results confirm that clusters representing classical MBCs are enriched for spike binding while B cells targeting internal proteins are enriched in activated naïve and innate-like B cell subsets.

3. SARS-CoV-2-Specific Ig Repertoire

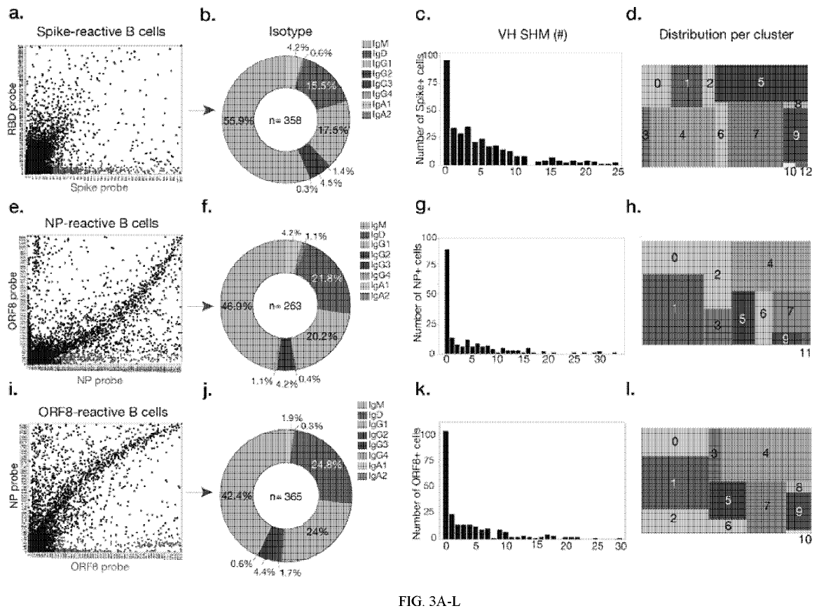

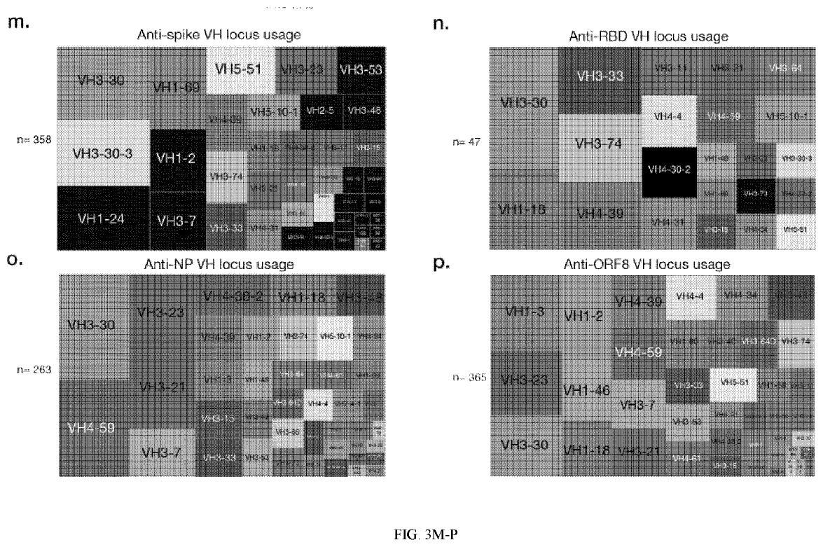

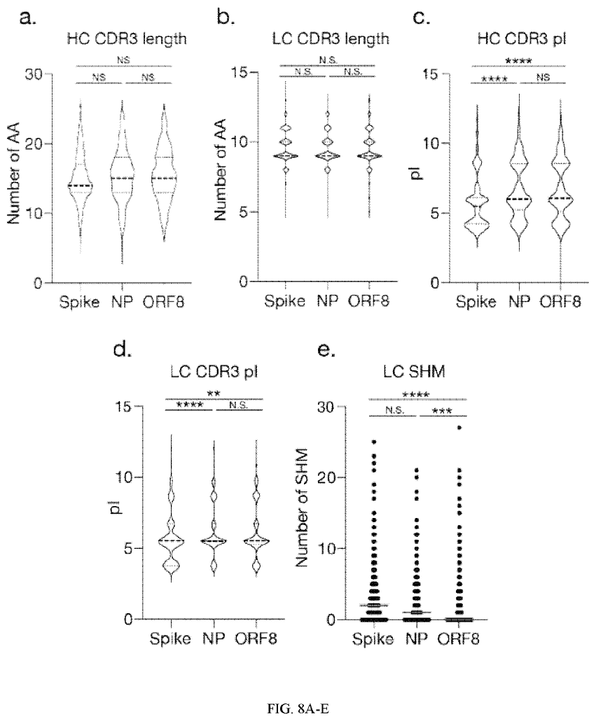

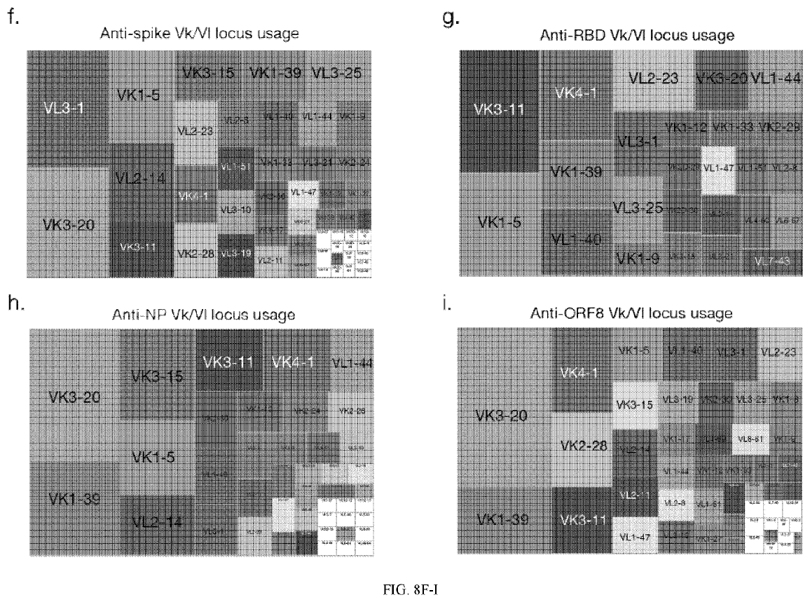

The properties of B cells targeting immunogenic targets such as ORF8 and NP compared to the spike are unknown. The inventors further analyzed isotype frequencies, VH SHM, VII gene usages, and frequencies of B cells against these targets within distinct B cell subsets. The majority of antigen-specific B cells were of the IgM isotype with a limited degree of CSR. There were no major differences between the isotypes of B cells specific to these distinct targets, with the majority of class-switched cells being of the IgG1 isotype. Consistent with a de novo response against the novel SARS-CoV-2, the inventors observed that the majority of antigen-specific B cells had little to no VH SHM, though spike-reactive B cells displayed slightly increased amounts of SHM. Spike-specific B cells were primarily enriched in MBC and LLPC-like clusters 4, 5, 7, 9, and 12 while NP- and ORF8-specific B cells were largely found within naïve- and innate-like clusters but also within MBC clusters (FIG. 3a-1). Lastly, the inventors did not observe differences in heavy chain (HC) or light chain (LC) complementarity determining region 3 length by antigen targeting (FIG. 8a-b), though the inventors did observe that HC and LC isoelectric points (pI) for spike-reactive B cells were generally lower than NP- or ORF8-reactive B cells (FIG. 8c-d), and LC SHM was greater for spike-reactive B cells (FIG. 8e).

The inventors next analyzed the VH gene usages of spike-, NP-, and ORF8-specific B cells and identified the most common VH usages per reactivity (represented by larger squares on each tree map) as well as shared VH usages across reactivities (shown by matching colors; FIG. 3m-p). Strikingly, the inventors identified usage of particular VH gene loci that did not overlap between spike- and RBD-reactive B cells (shown in black). VH1-24, VH3-7, and VH3-9 were the highest represented VH gene usages exclusively associated with non-RBD spike reactivity, and VH1-24 usage was enriched in cluster 7, an MBC-like cluster (FIG. 3m-n, FIG. 6b). These results were confirmed by mAb data, which identified spike-specific mAbs utilizing VH1-24 and VH3-7 that did not bind to the RBD (Extended Data Table 3). Unique LC V gene usages were also evident amongst antigen-specific cells (FIG. 8f-i).

Finally, public B cell clones were of interest as the epitopes bound can be targeted by multiple people and thus represent important vaccine targets. The inventors identified five novel public clones from this dataset, three of which were present in two separate subjects, one that was present amongst three subjects, and one amongst four subjects (Extended Data Table 4). Four of the clonal pools were specific to the spike protein, and the remaining clone to NP. The majority of clonal pool members were identified in MBC-like clusters 3, 4, 5, 7, and 9, suggesting that B cells specific to public epitopes can be established within stable MBC compartments.

4. Monoclonal Antibody Binding and Neutralization

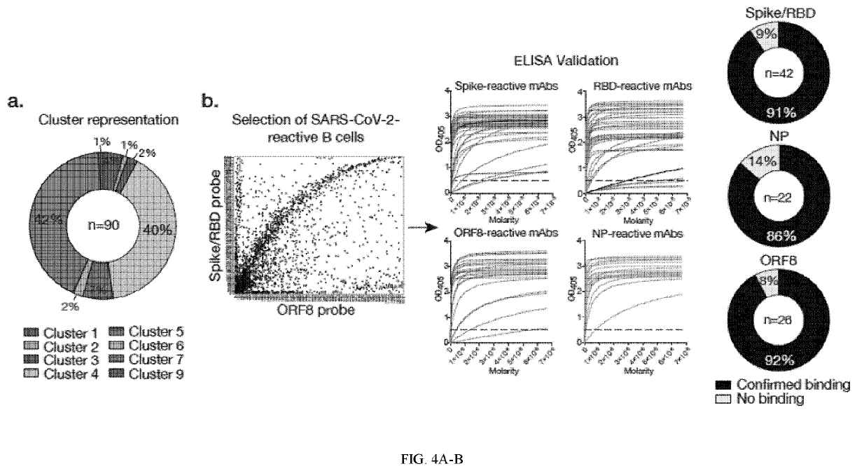

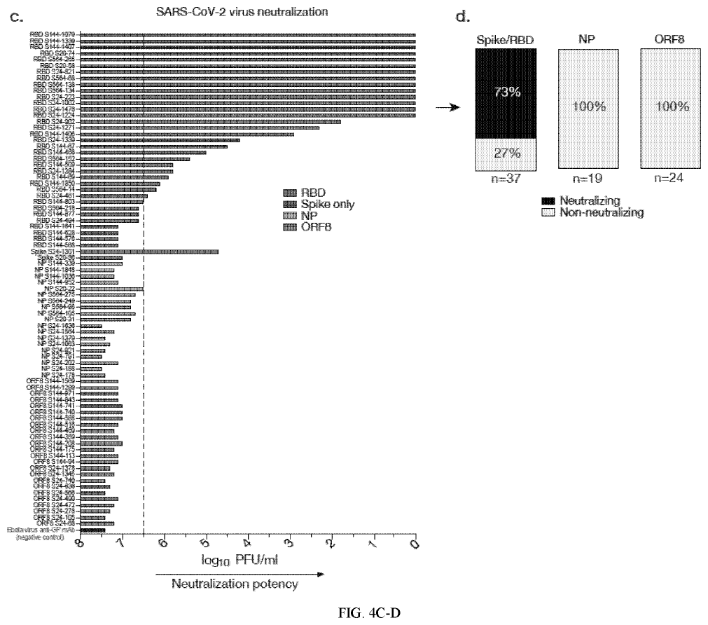

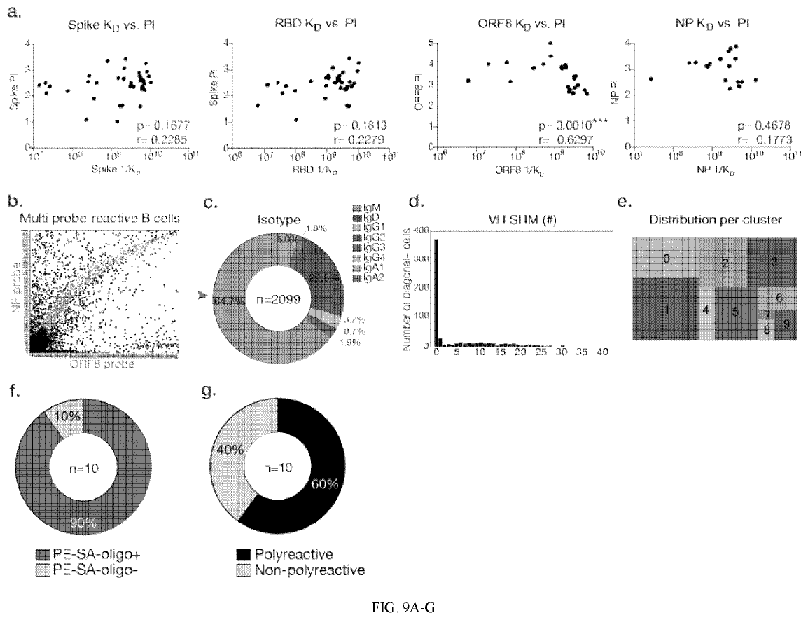

To simultaneously validate the specificity of the approach and investigate the properties of mAbs targeting distinct SARS-CoV-2 viral epitopes, the inventors synthesized and characterized the binding and neutralization ability of 90 mAbs from the single cell dataset (Extended Data Table 3). B cells exhibiting variable probe binding intensities toward distinct antigens were chosen as candidates for mAb generation, as well as B cells that tended to bind multiple probes (exhibiting non-specificity or polyreactivity). MAbs cloned were representative of various clusters, reactivities, VH gene usages, mutational load, and isotype usages (FIG. 4a, Extended Data Table 3). Representative mAbs generated from cells specific to the spike, NP, and ORF8 exhibited high affinity by ELISA, though probe intensities did not meaningfully correlate with apparent affinity (K_{D}) (FIG. 4b, 9a). Only a small percentage of cloned mAbs to the spike, NP, and ORF8 exhibited non-specific binding (FIG. 4b). Notably, non-specific multi-probe-binding cells were reactive to the PE-SA-oligo probe conjugate and were largely polyreactive (FIG. 9b-g).

While mAbs targeting the RBD of the spike are typically neutralizing, little is known regarding the neutralization capabilities of mAbs targeting non-RBD regions of the spike, ORF8 and NP. The inventors addressed the neutralization ability of all synthesized mAbs using a live virus plaque assay and determined that all mAbs cloned against NP and ORF8 were non-neutralizing, while mAbs against the RBD and other epitopes of the spike were largely neutralizing at varying degrees of potency (FIG. 4c-d). As anti-spike mAbs were predominantly neutralizing and enriched in memory, these MBC subsets may serve as a biomarker for superior immunity to SARS-CoV-2.

5. Antigen Targeting and Clinical Features

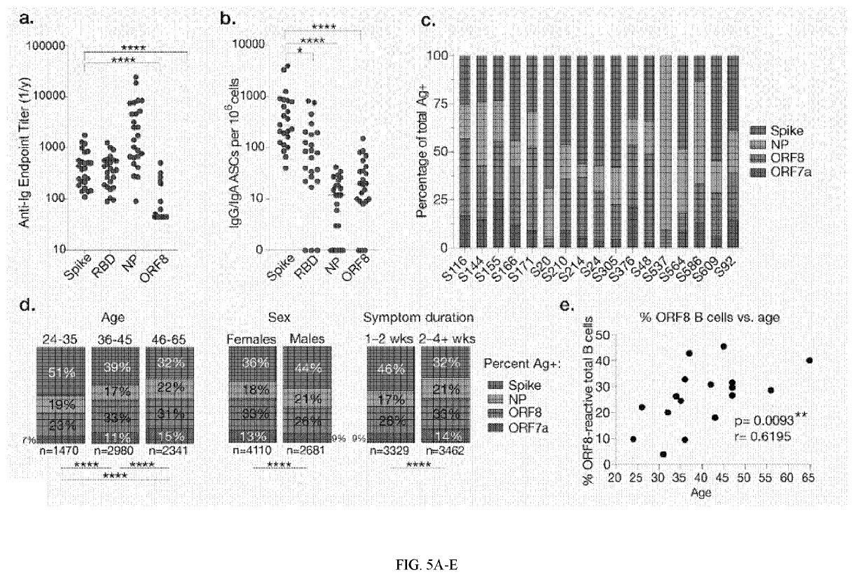

Previous studies from the inventors' group and others have suggested serum antibody titers correlate with sex, SARS-CoV-2 severity, and age^{6,14,23}. The inventors therefore investigated the frequencies of SARS-CoV-2-reactive B cells to assess whether reactivity toward particular SARS-CoV-2 antigens correlated with clinical parameters. By both serology and ELISpot, the inventors identified that B cell responses against the spike/RBD and NP were immunodominant, though ORF8 antigen targeting was substantial (FIG. 5a, b). Consistent with the single cell dataset, spike-specific B cells were enriched in memory by ELISpot (FIG. 5b).

The inventors next analyzed the distribution of B cell subsets and frequencies of B cells specific to the spike, NP, ORF7a, and ORF8 in sets of patients stratified by age, sex, and duration of symptoms from the single cell dataset. The inventors normalized antigen probe signals by a centered log-ratio transformation individually for each subject; all B cells were clustered into multiple probe hit groups according to their normalized probe signals, and cells that were negative to all probes or positive to all probes (non-specific) were excluded from the analysis. The inventors identified substantial variation in antigen targeting amongst individual subjects (FIG. 5c). As subject age increased, the percentages of spike-reactive B cells relative to B cells targeting internal proteins decreased, and age positively correlated with increased percentages of ORF8-reactive B cells (FIG. 5d-e). Similarly, female subjects and subjects experiencing a longer duration of symptoms displayed reduced spike targeting relative to internal proteins (FIG. 5d). Consistent with spike-reactive B cells enriched in MBC clusters, patient who were younger, male, or experienced a shorter duration of symptoms exhibited increased targeting of the spike and increased proportions of MBC subsets (FIG. 5d, f). Accordingly, older patients, female patients, and patients with a longer duration of symptoms exhibited reduced levels of VH gene SHM (FIG. 5g-i).

In summary, this study highlights the diversity of B cell subsets expanded upon novel infection with SARS-CoV-2 Using this approach, the inventors identified that B cells against the spike, ORFS, and NP differ in their ability to neutralize, derive from functionally distinct and differentially adapted B cell subsets, and correlate with clinical parameters such as age, sex, and symptom duration.

B. Discussion

The COVID-19 pandemic continues to pose one of the greatest public health and policy challenges in modern history, and robust data on long-term immunity is critically needed to evaluate future decisions regarding COVID-19 responses. This approach combines three powerful aspects of B cell biology to address human immunity to SARS-CoV-2: B cell transcriptome, Ig sequencing, and recombinant mAb characterization. This approach enables the identification of potently neutralizing antibodies and the characteristics of the B cells that generate them. Importantly, the inventors showed that antibodies targeting key protective spike epitopes are enriched within canonical MBC populations.

Identification of multiple distinct subsets of innate-like B cells, MBCs, and apparent LLPC precursors illustrates the complexity of the B cell response to SARS-CoV-2, revealing an important feature of the immune response against a novel pathogen. The B cell clusters herein may provide biomarkers in the form of distinct B cell populations that can be used to evaluate future responses to various vaccine formulations. In particular, the identification of LLPC precursors in the blood following infection and vaccination has been long sought after, as they serve as a bonafide marker of long-lived immunity^{24,25}. Future studies elucidating distinct identities and functions of these subsets are necessary and will provide key insights into B cell immunology.

The inventors identified that older patients, female patients, and patients experiencing a longer duration of symptoms tended to display reduced proportions of MBC clusters and reduced VH SHM, consistent with a previous study that identified limited germinal center formation upon SARS-CoV-2 infection²⁶. Notably, older patients had increased percentages of ORFS-specific B cells, which the inventors identified as exclusively non-neutralizing. Mechanistically, these observations may be explained by reduced adaptability of B cells or increased reliance on CD4 T cell help for B cell activation, which have been observed in aged individuals upon viral infections^{27,28}. Furthermore, T cell responses to SARS-CoV-2 ORF proteins are prevalent in convalescent COVID-19 patients, and recent studies suggest impaired T cell responses in aged COVID-19 patients impact antibody responses^{10,29,30,42}. More research is warranted to definitively determine whether B cell targeting of distinct SARS-CoV-2 antigens correlates with age and disease severity. Addressing these questions will be critical for determining correlates of protection and developing a vaccine capable of protecting the most vulnerable populations.

C. Materials & Methods

1. Study Cohort and Sample Collection

Clinical information for patients included in the study is detailed in Extended Data Table 1 and Extended Data Table 2. No statistical methods were used to predetermine sample size, experiments were not randomized, and investigators were unblinded. Leukoreduction filter donors were 18 years of age or older, eligible to donate blood as per standard University of Chicago Medicine Blood Donation Center guidelines, had a documented COVID-19 polymerase chain reaction (PCR) positive test, and complete resolution of symptoms at least 28 days prior to donation. PBMCs were collected from leukoreduction filters within 2 hours post-collection and flushed from the filters using sterile 1× Phosphate-Buffered Saline (PBS, Gibco) supplemented with 0.2% Bovine Serum Albumin (BSA, Sigma). Lymphocytes were purified by Lymphoprep Ficoll gradient (Thermo Fisher) and contaminating red blood cells were lysed by ACK buffer (Thermo Fisher). Cells were frozen in Fetal Bovine Serum (FBS, Gibco) with 10% Dimethyl sulfoxide (DMSO, Sigma) prior to downstream analysis. On the day of sorting, B cells were enriched using the human pan B cell EasySep™ enrichment kit (STEMCELL).

2. Recombinant Proteins and Probe Generation

Sequences for the spike and RBD proteins as well as details regarding their expression and purification have been previously described^{31,32}. Proteins were biotinylated for 2 hours on ice using EZ-Link™ Sulfo-NHS-Biotin, No-Weigh™ Format (Thermo Fisher) according to the manufacturer's instructions, unless previously Avi-tagged and biotinylated (ORF7a and ORF8 proteins, Fremont laboratory). Truncated cDNAs encoding the Ig-like domains of ORF7a and ORF8 were inserted into the bacterial expression vector pET-21(a) in frame with a biotin ligase recognition sequence at the c-terminus (GLND1FEAQKIEWHE). Soluble recombinant proteins were produced as described previously³³. In brief, inclusion body proteins were washed, denatured, reduced, and then renatured by rapid dilution following standard methods³⁴. The refolding buffer consisted of 400 mM arginine, 100 mM Tris-HCl, 2 mM EDTA, 200 μM ABESF, 5 mM reduced glutathione, and 500 μM oxidized glutathione at a final pH of 8.3. After 24 hours, the soluble-refolded protein was collected over a 10 kDa ultrafiltration disc (EMD Millipore, PLGC07610) in a stirred cell concentrator and subjected to chromatography on a HiLoad 26/60 Superdex S75 column (GE Healthcare). Site-specific biotinylation with BirA enzyme was done following the manufacture's protocol (Avidity) except that the reaction buffer consisted of 100 mM Tris-HCl (pH 7.5) 150 mM NaCl, with 5 mM MgCl2 in place of 0.5 M Bicine at pH 8.3. Unreacted biotin was removed by passage through a 7K MWCO desalting column (Zeba spin, Thermo Fisher). Full-length SARS-CoV-2 NP was cloned into pET21a with a hexahistidine tag and expressed using BL21(DE3)-RIL E. coli in Terrific Broth (bioWORLD). Following overnight induction at 25° C., cells were lysed in 20 mM Tris-HCl pH 8.5, 1 M NaCl, 5 mM β-mercaptoethanol, and 5 mM imidazole for nickel-affinity purification and size exclusion chromatography. Biotinylated proteins were then conjugated to Biolegend TotalSeq™ PE streptavidin-(PE-SA) oligos at a 0.72:1 molar ratio of antigen to PE-SA. The amount of antigen was chosen based on a fixed amount of 0.5 μg PE-SA and diluted in a final volume of 10 μL. PE-SA was then added gradually to 10 μl biotinylated proteins 5 times on ice, 1 μl PE-SA (0.1 mg/ml stock) every 20 minutes for a total of 5 μl (0.5 μg) PE-SA. The reaction was then quenched with 5 μl 4 mM Pierce™ biotin (Thermo Fisher) for 30 minutes for a total probe volume of 20 μL. Probes were then used immediately for staining.

3. Antigen-Specific B Cell Sorting

PBMCs were thawed and B cells were enriched using EasySep™ pan B cell magnetic enrichment kit (STEMCELL). B cells were stained with a panel containing CD19 PE-Cy7 (Biolegend), IgM APC (Southern Biotech), CD27 BV605 (Biolegend), CD38 BB515 (BD Biosciences), and CD3 BV510 (BD Biosciences). B cells were stained with surface stain master mix and each COVID-19 antigen probe for 30 minutes on ice in 1×PBS supplemented with 0.2% BSA and 2 mM Pierce Biotin. Cells were stained with probe at a 1:100 dilution (NP, ORF7a, ORF8, RBD) or 1:200 dilution (spike). Cells were subsequently washed with 1×PBS BSA and stained with Live/Dead BV510 (Thermo Fisher) in 1×PBS for 15 minutes. Cells were washed again and re-suspended at a maximum of 4 million cells/mL in 1×PBS supplemented with 0.2% BSA and 2 mM Pierce Biotin for downstream cell sorting using the MACSQuantTyto cartridge sorting platform (Miltenyi). Cells that were viable/CD19⁺/antigen-PE⁺ were sorted as probe positive. The PE⁺ gate was drawn by use of FMO controls. Cells were then collected from the cartridge sorting chamber and used for downstream 10× Genomics analysis.

4. 10× Genomics Library Construction

VDJ, 5′, and probe feature libraries were prepared using the 10× Chromium System (10× Genomics, Pleasanton, CA). The Chromium Single Cell 5′ Library and Gel Bead v2 Kit, Human B Cell V(D)J Enrichment Kit, and Feature Barcode Library Kit were used. All steps were followed as listed in the manufacturer's instructions. Specifically, user guide CG000186 Rev D was used. Final libraries were pooled and sequenced using the NextSeq550 (Illumina, San Diego, CA) with 26 cycles apportioned for read 1, 8 cycles for the i7 index, and 134 cycles for read 2.

5. Computational Analyses for Single Cell Sequencing Data

The inventors adopted Cell Ranger (version 3.0.2) for raw sequencing processing, including 5′ gene expression analysis, antigen probe analysis, and immunoprofiling analysis of B cells. Based on Cell Ranger output, the inventors performed downstream analysis using Seurat (version 3.2.0, an R package, for transcriptome, cell surface protein and antigen probe analysis) and IgBlast (version 1.15, for immunoglobulin gene analysis). For transcriptome analysis, Seurat was used for cell quality control, data normalization, data scaling, dimension reduction (both linear and non-linear), clustering, differential expression analysis, batch effects correction, and data visualization. Unwanted cells were removed according to the number of detectable genes (number of genes <200 or >2500 were removed) and percentage of mitochondrial genes for each cell. A soft threshold of percentage of mitochondrial genes was set to the 95^{th} percentile of the current dataset distribution, and the soft threshold was subject to a sealing point of 10% as the maximum threshold in the case of particularly poor cell quality. Transcriptome data were normalized by a log-transform function with a scaling factor of whereas cell surface protein and antigen probe were normalized by a centered log-ratio (CLR) normalization. The inventors used variable genes in principal component analysis (PCA) and used the top 15 principal components (PCs) in non-linear dimension reduction and clustering. High-quality cells were then clustered by Louvain algorithm implemented in Seurat under the resolution of 0.6. Differentially expressed genes for each cell cluster were identified using a Wilcoxon rank-sum test implemented in Seurat. Batch effects correction analysis was performed using an Anchor method implemented in Seurat to remove batch effects across different datasets. All computational analyses were performed in R (version 3.6.3).

6. Trajectory and Pseudotime Analyses

Trajectory analyses were performed using Monocle 3 (version 0.2.2)^{35,36}, Seurat 3, and the SeuratWrappers package (version 0.2.0)³⁷. Cells from multiple subjects were integrated to remove batch effects using Seurat, and all cells were clustered into two non-connected partitions. The inventors then performed trajectory analysis on the main partition containing the majority of the cells and clusters (clusters 0-11). Pseudotime analysis of cells was also inferred from this major partition using Monocle3. The root node of the pseudotime analysis was set to cluster 2, a naïve B cell subset with the lowest degree of VH gene SHIM and CSR.

7. Selection of Antibodies for mAb Synthesis

Representative antibodies from each subject were chosen for synthesis by choosing random samplings of B cells that bound to a given antigen probe with higher intensity relative to all other probes. B cells with varying ranges of probe-binding intensities were chosen for confirmation by ELISA. B cells binding to all probes in a polyreactive manner were also chosen and validated for polyreactivity by polyreactivity ELISA (see methods below).

8. Monoclonal Antibody Generation

Immunoglobulin heavy and light chain genes were obtained by 10× Genomics VDJ sequencing analysis and monoclonal antibodies (mAbs) were synthesized by Integrated DNA Technologies. Cloning, transfection, and mAb purification have been previously described³⁸. Briefly, sequences were cloned into human IgG1 expression vectors using Gibson assembly, and heavy and light genes were co-transfected into 293T cells (Thermo Fisher). Secreted mAbs were then purified from the supernatant using protein A agarose beads (Thermo Fisher).

9. Enzyme-Linked Immunosorbent Assay (ELISA)

High-protein binding microtiter plates (Costar) were coated with recombinant SARS-CoV-2 proteins at 2 μg/ml in 1× PBS overnight at 4° C. Plates were washed the next morning with 1×PBS 0.05% Tween and blocked with 1× PBS containing 20% fetal bovine serum (FBS) for 1 hour at 37° C. Antibodies were then serially diluted 1:3 starting at 10 μg/ml and incubated for 1 hour at 37° C. Horseradish peroxidase (HRP)-conjugated goat anti-human IgG antibody diluted 1:1000 (Jackson Immuno Research) was used to detect binding of mAbs, and plates were subsequently developed with Super Aquablue ELISA substrate (eBiosciences). Absorbance was measured at 405 nm on a microplate spectrophotometer (BioRad). To standardize the assays, control antibodies with known binding characteristics were included on each plate and the plates were developed when the absorbance of the control reached 3.0° Dos units. Data are representative of 2-4 independent experiments with 2 technical replicates.

10. Polyreactivity ELISA

Polyreactivity ELISAs were performed as previously described^{39,40}. High-protein binding microtiter plates (Costar) were coated with 10 μg/ml calf thymus dsDNA (Thermo Fisher), 2 μm/ml Salmonella enterica serovar Typhimurium flagellin (Invitrogen), 5 μg/ml human insulin (Sigma-Aldrich), 10 μg/ml KLH (Invitrogen), and 10 μg/ml Escherichia coli LPS (Sigma-Aldrich) in 1×PBS. Plates were coated with 10 μg/ml cardiolipin in 100% ethanol and allowed to dry overnight. Plates were washed with water and blocked with 1× PBS/0.05% Tween/1 mM EDTA. MAbs were diluted 1 μg/ml in PBS and serially diluted 4-fold, and added to plates for 1.5 hours. Goat anti-human IgG-HRP (Jackson Immunoresearch) was diluted 1:2000 in PBS/0.05% Tween/1 mM EDTA and added to plates for 1 hour. Plates were developed with Super Aquablue ELISA substrate (eBioscience) until the positive control mAb, 3H9⁴¹, reached an OD₄₀₅ of 3. MAbs were screened once for polyreactivity with 2 technical replicates.

11. Memory B Cell Stimulations and Enzyme-Linked Immunospot Assays (ELISpot)

MBC stimulations were performed on PBMCs collected from subjects in the convalescent cohort. To induce MBC differentiation into antibody secreting cells, 1×10⁶ PBMCs were stimulated with 10 ng/ml Lectin Pokeweed Mitogen (Sigma-Aldrich), 1/100,000 Protein A from Staphylococcus aureus, Cowan Strain (Sigma-Aldrich), and 6 μg/ml CpG (Invitrogen) in complete RPMI in an incubator at 37° C./5% CO₂ for 5 days. After stimulation, cells were counted and added to ELISpot white polystyrene plates (Thermo Fisher) coated with 4 μg/ml of SARS-CoV-2 spike that were blocked with 200 μl of complete RPMI. ELISpot plates were incubated with cells for 16 hours overnight in an incubator at 37° C./5% CO₂. After the overnight incubation, plates were washed and incubated with anti-IgG-biotin and/or anti-IgA-biotin (Mabtech) for 2 hours at room temperature. After secondary antibody incubation, plates were washed and incubated with streptavidin-alkaline phosphatase (Southern Biotech) for 2 hours at room temperature. Plates were washed and developed with NBT/BCIP (Thermo Fisher Scientific) for 2-10 minutes, and reactions were stopped by washing plates with distilled water and allowed to dry overnight before counting. Images were captured with Immunocapture 6.4 software (Cellular Technology Ltd.), and spots were manually counted. Experiments were performed once with 2 technical replicates due to limited cell availability.

12. Neutralization Assay

The SARS-CoV-2/UW-001/Human/2020/Wisconsin (UW-001) virus was isolated from a mild case in February 2020 and used to assess neutralization ability of mAbs. Virus (˜500 plaque-forming units) was incubated with each mAb at a final concentration of 10 μg/ml. After a 30-minute incubation at 37° C., the virus/antibody mixture was used to inoculate Vero E6/TMPRSS2 cells seeded a day prior at 200,000 cells per well of a TC12 plate. After 30 minutes at 37° C., cells were washed 3 times to remove any unbound virus, and media containing antibody (10 μg/ml) was added back to each well. 2 days after inoculation, cell culture supernatant was harvested and stored at −80° C. until needed. A non-relevant Ebola virus GP mAb and PBS were used as controls.

To determine the amount of virus in the cell culture supernatant of each well, a standard plaque-forming assay was performed. Confluent Vero E6/TMPRSS2 cells in a TC12 plate were infected with supernatant (undiluted, 10-fold dilutions from 10⁻¹ to 10⁻⁵) for 30 minutes at 37° C. After the incubation, cells were washed 3 times to remove unbound virus and 1.0% methylcellulose media was added over the cells. After an incubation of 3 days at 37° C., the cells were fixed and stained with crystal violet solution in order to count the number plaques at each dilution and determine virus concentration given as plaque-forming units (PFU)/ml. A stringent cutoff for neutralization was chosen as 100-fold greater neutralization relative to the negative control mAb. MAbs were screened once for neutralization.

13. Statistical Analysis

All statistical analyses were performed using Prism (GraphPad Prism version 8.0) or IMP Pro software (version 15.1.0). Sample sizes (n) are indicated directly in the figures or in the corresponding figure legends and specific tests for statistical significance used are indicated in the corresponding figure legends. P values less than or equal to 0.05 were considered significant. *p<0.05, **p<0.01, ***p<0.001, ****p<0.0001. All measures analyzed within the single cell dataset were analyzed repeatedly within the same integrated dataset, and independent preparations of mAb confirmed consistent binding patterns

Extended Data Table 1. Individual patient information.

| | | | | Duration | Symptom | |
|---|---|---|---|---|---|---|
| | | | | of | start to | |
| Subject | | | | symptoms | donation | |
| ID | Age | Sex | Reported symptoms* | (days) | (days) | Available data |
| 24 | 34 | M | Fatigue, cough, SOB, SC, fever, headache, BAP, | 12 | 41 | Single cell probe binding, ELISPOT, serology |
| | | | diarrhea, LOS, LOT | | | |
| 20 | 31 | M | Fatigue, cough, SOB, SC, fever, headache, BAP, | 19 | 48 | Single cell probe binding, ELISPOT, serology |
| | | | LOS, LOT | | | |
| 564 | 24 | F | Fatigue, cough, SOB, SC, ST, fever, headache, | 32 | 60 | Single cell probe binding, ELISPOT, serology |
| | | | BAP, diarrhea, LOS, LOT | | | |
| 144 | 56 | M | Fatigue, cough, SC, ST, headache, BAP, LOS | 23 | 54 | Single cell probe binding, ELISPOT, serology |
| 214 | 47 | M | Fatigue, cough, SOB, SC, ST, headache, BAP, | 24 | 59 | Single cell probe binding, ELISPOT, serology |
| | | | LOS, LOT | | | |
| 171 | 37 | F | Fatigue, cough, SOB, SC, fever, headache, BAP, | 16 | 44 | Single cell probe binding, ELISPOT, serology |
| | | | diarrhea, LOS, LOT | | | |
| 92 | 35 | M | Fatigue, cough, SC, ST, fever, headache, BAP | 16 | 47 | Single cell probe binding, ELISPOT, serology |
| 48 | 45 | F | Fatigue, cough, SOB, SC, ST, fever, headache, AP, | 8 | 40 | Single cell probe binding, ELISPOT, serology |
| | | | diarrhea, LOS, LOT | | | |
| 537 | 36 | M | Fatigue, cough, fever, BAP | 14 | 59 | Single cell probe binding, ELISPOT, serology |
| 586 | 32 | F | Fatigue, cough, SOB, SC, headache, BAP, AP, | 17 | 61 | Single cell probe binding, ELISPOT, serology |
| | | | diarrhea | | | |
| 210 | 47 | M | Fatigue, cough, SOB, fever, headache, BAP, LOS, | 7 | 41 | Single cell probe binding, ELISPOT, serology |
| | | | LOT | | | |
| 376 | 36 | F | Diarrhea, LOS, LOT | 7 | 48 | Single cell probe binding, ELISPOT, serology |
| 305 | 43 | F | Fatigue, cough, SC, ST, fever, headache, BAP, | 4 | 47 | Single cell probe binding, ELISPOT, serology |
| | | | LOS, LOT | | | |
| 116 | 65 | F | Cough, SOB, fever, LOS, LOT | 18 | 49 | Single cell probe binding, ELISPOT, serology |
| 166 | 42 | F | Fatigue, cough, SOB, SC, fever, headache, BAP, | 17 | 55 | Single cell probe binding, ELISPOT, serology |
| | | | diarrhea, LOS, LOT | | | |
| 155 | 47 | F | Fatigue, cough, SOB, ST, fever, BAP, LOS, LOT | 29 | 64 | Single cell probe binding, serology |
| 609 | 26 | F | Fatigue, SOB, ST, fever, headache, BAP, LOS, | 7 | 57 | Single cell probe binding, serology |
| | | | LOT | | | |
| 282 | 34 | F | Fatigue, cough, SOB, fever, BAP, AP, LOS | 24 | 54 | ELISPOT, serology |
| 326 | 36 | F | Fatigue, cough, SC, fever, headache, BAP, AP, | 15 | 47 | ELISPOT, serology |
| | | | LOS, LOT | | | |
| 356 | 51 | F | Fatigue, cough, ST, fever, headache, BAP, AP, | 14 | 43 | ELISPOT, serology |
| | | | diarrhea, LOS, LOT | | | |
| 373 | 48 | M | Fatigue, fever, headache, BAP | 7 | 39 | ELISPOT, serology |
| 402 | 32 | F | Fatigue, cough, SOB, fever, headache, BAP, AP, | 11 | 44 | ELISPOT, serology |
| | | | diarrhea, LOS, LOT | | | |
| 65 | 40 | F | Fatigue, SC, fever, headache, BAP, diarrhea, LOS, | 13 | 47 | ELISPOT, serology |
| | | | LOT | | | |
| 423 | 58 | M | Fatigue | 5 | 38 | ELISPOT, serology |
| 558 | 56 | F | Fatigue, cough, SOB, LOS | 11 | 46 | ELISPOT, serology |

*SOB = shortness of breath; SC = sinus congestion; ST = sore throat; BAP = body aches and pain; AP = abdominal pain; LOS = loss of smell; LOT = loss of taste

Extended Data Table 3.
MAbs generated from single B cell heavy and light chain gene sequences.

| | | | | | | | | HC CDR3 | LC CDR3 | |
|---|---|---|---|---|---|---|---|---|---|---|
| B cell | Clonal | | | DH | JH | LC V | LC J | AA | AA | |
| clone | Pool | Antigen | VH gene | gene | gene | gene | gene | sequence | sequence | Cluster |
| S144- | 1 | Spike | 3-23*01 | N/A | 4*02 | k3-20* | k1*01 | AKGSSTA | QEYGSSRM | 5 |
| 121 | | | | | | 01 | | RPYYFDY | (SEQ ID | |
| | | | | | | | | (SEQ ID | NO: 1802) | |
| | | | | | | | | NO: 1801) | | |
| S155- | 1 | Spike | 3-23*01 | 6-13*01 | 4*02 | k3-20* | k1*01 | VKGSAAA | QQYGNSRI | 3 |
| 37 | | | | | | 01 | | RPYYFDY | (SEQ ID | |
| | | | | | | | | (SEQ ID | NO: 1804) | |
| | | | | | | | | NO: 1803) | | |
| S210- | 2 | Spike | 3-30- | 1-7*01 | 4*02 | k3-20* | k3*01 | ARGHGNY | QQYGSSPLT | 5 |
| 896 | | | 3*01 | | | 01 | | LTYFDY | (SEQ ID | |
| | | | | | | | | (SEQ ID | NO: 284) | |
| | | | | | | | | NO: 1805) | | |
| S376- | 2 | Spike | 3-30- | 1-26*01 | 4*02 | k3-20* | k4*01 | ARGRGNY | QQYGGSLT | 7 |
| 2486 | | | 3*01 | | | 01 | | FTYFDY | (SEQ ID | |
| | | | | | | | | (SEQ ID | NO: 1807) | |
| | | | | | | | | NO: 1806) | | |
| S166- | 3 | Spike | 3-7*03 | 6-19*01 | 4*02 | 13-1* | 12*01 | ARDSIAV | QAWDSSTVV | 5 |
| 2620 | | | | | | 01 | | AGGLDY | (SEQ ID | |
| | | | | | | | | (SEQ ID | NO: 698) | |
| | | | | | | | | NO: 1808) | | |
| S166- | 3 | Spike | 3-7*03 | 6-19*01 | 4*02 | 13-1* | 12*01 | ARDGIAV | QAWDSSTVV | 4 |
| 1318 | | | | | | 01 | | AGGFDY | (SEQ ID | |
| | | | | | | | | (SEQ ID | NO: 698) | |
| | | | | | | | | NO: 1809) | | |
| S171- | 3 | Spike | 3-7*01 | 6-19*01 | 4*02 | 13-1* | 12*01 | ARDGIAV | QAWDSSTVV | 9 |
| 1150 | | | | | | 01 | | AGGLDY | (SEQ ID | |
| | | | | | | | | (SEQ ID | NO: 698) | |
| | | | | | | | | NO: 1810) | | |
| S210- | 3 | Spike | 3-7*01 | 6-19*01 | 4*02 | 13-1* | 12*01 | ARDGIAV | QAWDSSTSVV | 4 |
| 852 | | | | | | 01 | | AGGFDY | (SEQ ID | |
| | | | | | | | | (SEQ ID | NO: 1811) | |
| | | | | | | | | NO: 1809) | | |
| S305- | 3 | Spike | 3-7*03 | 6-19*01 | 4*02 | 13-1* | 12*01 | ARDSIAV | QAWDSSTNVV | 5 |
| 968 | | | | | | 01 | | AGGFDY | (SEQ ID | |
| | | | | | | | | (SEQ ID | NO: 1813) | |
| | | | | | | | | NO: 1812) | | |
| S564- | 4 | NP | 3-7*01 | 1-26*01 | 4*02 | k3-15* | k2*01 | ARGDGSN | QQYNYWYT | 5 |
| 128 | | | | | | 01 | | SGIYFDS | (SEQ ID | |
| | | | | | | | | (SEQ ID | NO: 1815) | |
| | | | | | | | | NO: 1814) | | |
| S469- | 4 | NP | 3-7*03 | 6-6*01 | 4*02 | k3-15* | k2*01 | ARGGGSS | QQYNYWYT | 5 |
| 373 | | | | | | 01 | | SGLYFES | (SEQ ID | |
| | | | | | | | | (SEQ ID | NO: 1815) | |
| | | | | | | | | NO: 1816) | | |
| S144- | 5 | Spike | 5-51*01 | 2-21*02 | 4*02 | k1-5* | k1*01 | ARLFCGG | QQYNTYPRT | 7 |
| 292 | | | | | | 01 | | DCPFDY | (SEQ ID | |
| | | | | | | | | (SEQ ID | NO: 1818) | |
| | | | | | | | | NO: 1817) | | |
| S2141- | 5 | Spike | 5-51*01 | 2-21*02 | 4*02 | k1-5* | k1*01 | ARQFCGG | QQYNSYPRT | 8 |
| 65 | | | | | | 01 | | DCPFDY | (SEQ ID | |
| | | | | | | | | (SEQ ID | NO: 1820) | |
| | | | | | | | | NO: 1819) | | |
| S144- | 5 | Spike | 5-51*01 | 3-10*01 | 4*02 | k1-5* | k2*01 | ARPNYYG | QQYNSYYT | 5 |
| 1364 | | | | | | 01 | | SGSPPGY | (SEQ ID | |
| | | | | | | | | (SEQ ID | NO: 1822) | |
| | | | | | | | | NO: 1821) | | |
| S210- | 5 | Spike | 5-51*01 | 3-10*01 | 4*02 | k3-20* | k1*01 | ARPFYYG | QLFGSSPTWT | 4 |
| 1139 | | | | | | 01 | | SESPPGY | (SEQ ID | |
| | | | | | | | | (SEQ ID | NO: 1824) | |
| | | | | | | | | NO: 1823) | | |

Extended Data Table 2. Distribution of clinical
parameters for patients included in the study.

| | |
|---|---|
| Median Age | 40 |
| Mean Age | 42 |
| Mode Age | 47 |
| Range Age | 24-65 |
| Number of Males | 9 |
| Number of Females | 16 |
| Median Duration of Symptoms (days) | 14 |
| Mean Duration of Symptoms (days) | 15 |
| Mode Duration of Symptoms (days) | 7 |
| Range Duration of Symptoms (days) | 4-32 |
| Median symptom start to donation (days) | 47 |
| Mean symptom start to donation (days) | 49 |
| Mode symptom start to donation (days) | 47 |
| Range symptom start to donation (days) | 38-64 |

Extended Data Table 3. MAbs generated from single
B cell heavy and light chain gene sequences.

| mAb ID | Specificity | Cluster | Isotype | # HC SHM | VH Gene | #LC SHM | Vk/L gene |
|---|---|---|---|---|---|---|---|
| S20-15 | Spike/RBD | 7 | IgG1 | 8 | VH 4-59 | 1 | VL 3-21 |
| S20-22 | NP | 9 | IgG1 | 7 | VH 4-4 | 4 | Vk 4-1 |
| S20-31 | NP | 7 | IgG4 | 30 | VH 1-24 | 22 | Vk 3-20 |
| S20-40 | NP | 2 | IgM | 0 | VH 4-4 | 1 | VL 2-14 |
| S20-58 | Spike/RBD | 4 | IgG1 | 5 | VH 4-30 | 2 | Vk 2-24 |
| S20-74 | Spike/RBD | 4 | IgG1 | 6 | VH 4-59 | 3 | VL 2-8 |
| S20-86 | Spike | 7 | IgG1 | 9 | VH 3-9 | 2 | VL 2-14 |
| S24-68 | ORF8 | 7 | IgG1 | 4 | VH 4-59 | 3 | VL 1-44 |
| S24-105 | ORF8 | 7 | IgG1 | 6 | VH 3-48 | 4 | Vk 3-20 |
| S24-178 | NP | 4 | IgG1 | 2 | VH 3-33 | 7 | VL 2-14 |
| S24-188 | NP | 7 | IgG3 | 2 | VH 1-69 | 3 | VL 2-14 |
| S24-202 | NP | 4 | IgG1 | 3 | VH 5-10 | 6 | Vk 3-11 |
| S24-278 | ORF8 | 7 | IgG1 | 3 | VH 1-2 | 1 | Vk 3-20 |
| S24-339 | Spike/RBD | 4 | Unknown | 5 | VH 3-49 | 1 | Vk 3-15 |
| S24-472 | ORF8 | 7 | IgG1 | 5 | VH 4-4 | 4 | VL 4-16 |
| S24-490 | ORF8 | 4 | IgM | 2 | VH 1-46 | 4 | Vk 3-20 |
| S24-494 | Spike/RBD | 6 | IgG3 | 0 | VH 4-39 | 0 | Vk 1-39 |
| S24-566 | ORF8 | 4 | IgG1 | 3 | VH 3-49 | 1 | Vk 2-28 |
| S24-636 | ORF8 | 1 | IgD | 1 | VH 3-7 | 4 | VL 8-61 |
| S24-740 | ORF8 | 7 | IgG1 | 5 | VH 1-3 | 1 | Vk 4-1 |
| S24-791 | NP | 7 | IgG1 | 4 | VH 4-59 | 6 | Vk 3-20 |
| S24-902 | Spike/RBD | 5 | IgG1 | 0 | VH 1-69 | 0 | VL 7-46 |
| S24-921 | NP | 7 | IgG1 | 8 | VH 4-59 | 7 | Vk 1-39 |
| S24-1063 | NP | 4 | IgG1 | 3 | VH 4-59 | 1 | Vk 3-20 |
| S24-1224 | Spike/RBD | 4 | IgG1 | 7 | VH 1-46 | 7 | VL 1-40 |
| S24-1271 | Spike/RBD | 7 | IgM | 6 | VH 3-66 | 6 | VL 3-1 |
| S24-1339 | Spike/RBD | 4 | IgG1 | 1 | VH 3-53 | 1 | Vk 3-20 |
| S24-1345 | ORF8 | 1 | IgD | 0 | VH 4-39 | 0 | Vk 1-13 |
| S24-1378 | ORF8 | 1 | IgM | 0 | VH 3-53 | 0 | VL 8-61 |
| S24-1379 | NP | 1 | IgG1 | 0 | VH 4-59 | 0 | VL 1-47 |
| S24-1384 | Spike/RBD | 7 | IgG1 | 2 | VH 3-48 | 4 | VL 3-21 |
| S24-1476 | Spike/RBD | 4 | IgG | 2 | VH 3-49 | 0 | Vk 3-15 |
| S24-1564 | NP | 4 | IgG1 | 10 | VH 4-59 | 4 | Vk 1-39 |
| S24-1636 | NP | 4 | IgG1 | 3 | VH 3-33 | 0 | Vk 3-11 |
| S24-1002 | Spike/RBD | 7 | IgM | 3 | VH 3-30 | 5 | Vk 1-13 |
| S24-1301 | Spike | 7 | IgG1 | 4 | VH 1-24 | 4 | VL 10-54 |
| S24-223 | Spike/RBD | 4 | IgM | 1 | VH 2-5 | 3 | VL 2-14 |
| S24-461 | Spike/RBD | 4 | IgG1 | 7 | VH 4-59 | 3 | VL 3-16 |
| S24-511 | NP | 5 | IgD | 0 | VH 3-30 | 0 | VL 3-1 |
| S24-788 | Spike/RBD | 5 | IgM | 0 | VH 3-33 | 1 | VL 3-1 |
| S24-821 | Spike/RBD | 4 | IgM | 4 | VH2-70 | 0 | Vk 1-5 |
| S144-67 | Spike/RBD | 7 | IgG1 | 7 | VH 5-51 | 5 | VL 1-40 |
| S144-69 | Spike/RBD | 4 | IgG1 | 2 | VH 5-51 | 3 | Vk 1-5 |
| S144-94 | ORF8 | 7 | IgG3 | 11 | VH 3-30 | 0 | Vk 2-28 |
| S144-113 | ORF8 | 7 | IgG1 | 9 | VH 3-23 | 6 | Vk 1-39 |
| S144-175 | ORF8 | 7 | IgG1 | 9 | VH 1-2 | 1 | VL 1-47 |
| S144-208 | ORF8 | 4 | IgG1 | 6 | VH 1-2 | 7 | VL 2-11 |
| S144-339 | NP | 4 | IgG1 | 11 | VH 3-21 | 7 | VK 3-20 |
| S144-359 | ORF8 | 4 | IgG3 | 5 | VH 3-23 | 5 | Vk 1-39 |
| S144-460 | Spike/RBD | 3 | IgA1 | 34 | VH 3-15 | 24 | Vk1D-17 |
| S144-466 | Spike/RBD | 7 | IgG3 | 6 | VH 5-51 | 6 | Vk 1-5 |
| S144-469 | ORF8 | 4 | IgG1 | 3 | VH 4-59 | 2 | Vk 2-28 |
| S144-509 | Spike/RBD | 7 | IgG1 | 3 | VH 5-51 | 1 | Vk 1-5 |
| S144-516 | ORF8 | 7 | IgG1 | 5 | VH 1-2 | 7 | VL 1-40 |
| S144-568 | Spike/RBD | 6 | IgA2 | 11 | VH 4-59 | 11 | Vk 3-20 |
| S144-576 | Spike/RBD | 4 | IgG1 | 3 | VH 1-69 | 2 | Vk 1-5 |
| S144-588 | ORF8 | 7 | IgG1 | 1 | VH 4-39 | 3 | VL 3-1 |
| S144-628 | Spike/RBD | 5 | IgA1 | 9 | VH 5-51 | 10 | VL 1-40 |
| S144-740 | ORF8 | 7 | IgG1 | 1 | VH 1-2 | 5 | Vk 3-20 |
| S144-741 | ORF8 | 4 | IgG1 | 5 | VH 1-2 | 1 | VL 1-44 |
| S144-803 | Spike/RBD | 4 | IgG1 | 5 | VH 5-51 | 3 | Vk 1-5 |
| S144-843 | ORF8 | 5 | Unknown | 20 | VH 3-30 | 8 | Vk 3-20 |
| S144-877 | Spike/RBD | 7 | IgG1 | 2 | VH 3-30 | 6 | Vk 1-33 |
| S144-952 | NP | 4 | IgM | 4 | VH 1-18 | 2 | Vk 4-1 |
| S144-971 | ORF8 | 7 | IgG1 | 6 | VH 3-64 | 3 | Vk 4-1 |
| S144-1036 | NP | 7 | IgG1 | 2 | VH 4-34 | 5 | Vk 4-1 |
| S144-1079 | Spike/RBD | 7 | IgG1 | 7 | VH 1-69 | 3 | Vk 3-20 |
| S144-1299 | ORF8 | 4 | IgG1 | 5 | VH 4-59 | 0 | VL 1-47 |
| S144-1339 | Spike/RBD | 4 | IgG1 | 12 | VH 1-2 | 5 | VL 2-14 |
| S144-1406 | Spike/RBD | 7 | IgG2 | 3 | VH 1-3 | 0 | Vk 1-5 |
| S144-1407 | Spike/RBD | 4 | IgG1 | 6 | VH 1-69 | 2 | Vk 1-5 |
| S144-1569 | ORF8 | 7 | IgG1 | 7 | VH 1-18 | 1 | VL 9-49 |
| S144-1641 | Spike/RBD | 7 | IgG1 | 4 | VH 5-51 | 9 | Vk 1-5 |
| S144-1827 | Spike/RBD | 3 | IgM | 20 | VH 3-7 | 5 | Vk 3-20 |
| S144-1848 | NP | 7 | IgG1 | 4 | VH 3-21 | 8 | VL 1-47 |
| S144-1850 | Spike/RBD | 7 | IgG1 | 2 | VH 3-23 | 3 | Vk 1-5 |
| S144-2234 | ORF8 | 4 | IgG1 | 4 | VH 1-69 | 3 | Vk 4-1 |
| S564-105 | NP | 4 | IgG1 | 5 | VH 4-61 | 2 | VL 2-14 |
| S564-14 | Spike/RBD | 4 | IgD | 3 | VH 3-7 | 0 | Vk 3-21 |
| S564-68 | Spike/RBD | 7 | IgG1 | 6 | VH 1-2 | 2 | VL 2-8 |
| S564-98 | NP | 7 | IgG3 | 0 | VH 4-59 | 3 | Vk 1-39 |
| S564-105 | NP | 4 | IgG1 | 5 | VH 4-61 | 2 | VL 2-14 |
| S564-134 | Spike/RBD | 7 | IgG1 | 2 | VH 1-2 | 6 | VL 2-8 |
| S564-138 | Spike/RBD | 4 | IgG1 | 8 | VH 1-2 | 1 | VL 2-14 |
| S564-152 | Spike/RBD | 7 | IgG1 | 4 | VH 3-33 | 4 | Vk 1-33 |
| S564-218 | Spike/RBD | 5 | IgM | 1 | VH 1-69 | 0 | VL 2-8 |
| S564-249 | NP | 4 | IgA1 | 19 | VH 3-64 | 19 | VL 2-14 |
| S564-265 | Spike/RBD | 7 | IgG1 | 4 | VH 1-2 | 3 | VL 2-8 |
| S564-275 | NP | 4 | IgM | 3 | VH 4-59 | 6 | Vk 1-39 |
| S564-287 | ORF8 | 4 | IgM | 1 | VH 1-2 | 3 | VL2-14 |

### E. References

The following references, to the extent that they provide exemplary procedural or other details supplementary to those set forth herein, are specifically incorporated herein by reference.
1 Chen, X. et al. Human monoclonal antibodies block the binding of SARS-CoV-2 spike protein to angiotensin converting enzyme 2 receptor. Cell Mol Immunol 17, 647-649, doi:10.1038/s41423-020-0426-7 (2020).
2 Wang, C. et al. A human monoclonal antibody blocking SARS-CoV-2 infection. Nat Commun 11, 2251, doi:10.1038/s41467-020-16256-y (2020).
3 Yi, C. et al. Key residues of the receptor binding motif in the spike protein of SARS-CoV-2 that interact with ACE2 and neutralizing antibodies. Cell Mol Immunol 17, 621-630, doi:10.1038/s41423-020-0458-z (2020).
4 Lan, J. et al. Structure of the SARS-CoV-2 spike receptor-binding domain bound to the ACE2 receptor. Nature 581, 215-220, doi:10.1038/s41586-020-2180-5 (2020).
5 Yan, R. et al. Structural basis for the recognition of SARS-CoV-2 by full-length human ACE2. Science 367, 1444-1448, doi:10.1126/science.abb2762 (2020).
6 Robbiani, D. F. et al. Convergent antibody responses to SARS-CoV-2 in convalescent individuals. Nature 584, 437-442, doi:10.1038/s41586-020-2456-9 (2020).
7 Wee, A. Z. et al. Broad neutralization of SARS-related viruses by human monoclonal antibodies. Science 369, 731-736, doi:10.1126/science.abc7424 (2020).
8 Kopecky-Bromberg, S. A., Martinez-Sobrido, L., Frieman, M., Baric, R. A. & Palese, P. Severe acute respiratory syndrome coronavirus open reading frame (ORF) 3b, ORF 6, and nucleocapsid proteins function as interferon antagonists. J Virol 81, 548-557, doi:10.1128/JVI.01782-06 (2007).
9 Lu, X., Pan, J., Tao, J. & Guo, D. SARS-CoV nucleocapsid protein antagonizes IFN-beta response by targeting initial step of IFN-beta induction pathway, and its C-terminal region is critical for the antagonism. Virus Genes 42, 37-45, doi:10.1007/s11262-010-0544-x (2011).
10 Grifoni, A. et al. Targets of T Cell Responses to SARS-CoV-2 Coronavirus in Humans with COVID-19 Disease and Unexposed Individuals. Cell 181, 1489-1501 e1415, doi:10.1016/j.cell.2020.05.015 (2020).
11 Ni, L. et al. Detection of SARS-CoV-2-Specific Humoral and Cellular Immunity in COVID-19 Convalescent Individuals. Immunity 52, 971-977 e973, doi:10.1016/j.immuni.2020.04.023 (2020).
12 Li, J. Y. et al. The ORF6, ORF8 and nucleocapsid proteins of SARS-CoV-2 inhibit type I interferon signaling pathway. Virus Res 286, 198074, doi:10.1016/j.virusres.2020.198074 (2020).
13 Atyeo, C. et al. Distinct Early Serological Signatures Track with SARS-CoV-2 Survival. Immunity 53, 524-532 e524, doi:10.1016/j.immuni.2020.07.020 (2020).
14 Guthmiller, J. J. et al. SARS-CoV-2 infection severity is linked to superior humoral immunity against the spike. bioRxiv, doi:10.1101/2020.09.12.294066 (2020).
15 Brouwer, P. J. M. et al. Potent neutralizing antibodies from COVID-19 patients define multiple targets of vulnerability. Science, doi:10.1126/science.abc5902 (2020).
16 Cao, Y. et al. Potent Neutralizing Antibodies against SARS-CoV-2 Identified by High-Throughput Single-Cell Sequencing of Convalescent Patients' B Cells. Cell 182, 73-84 e16, doi:10.1016/j.cell.2020.05.025 (2020).
17 Hansen, J. et al. Studies in humanized mice and convalescent humans yield a SARS-CoV-2 antibody cocktail. Science, doi:10.1126/science.abd0827 (2020).
18 Zhang, X. Regulatory functions of innate-like B cells. Cell Mol Immunol 10, 113-121, doi:10.1038/cmi.2012.63 (2013).
19 Palm, A. E. & Henry, C. Remembrance of Things Past: Long-Term B Cell Memory After Infection and Vaccination. Front Immunol 10, 1787, doi:10.3389/fimmu.2019.01787 (2019).
20 Laidlaw, B. J., Duan, L., Xu, Y., Vazquez, S. E. & Cyster, J. G. The transcription factor Hhex cooperates with the corepressor Tle3 to promote memory B cell development. Nat Immunol 21, 1082-1093, doi:10.1038/s41590-020-0713-6 (2020).
21 Lightman, S. M., Utley, A. & Lee, K. P. Survival of Long-Lived Plasma Cells (LLPC): Piecing Together the Puzzle. Front Immunol 10, 965, doi:10.3389/fimmu.2019.00965 (2019).
22 Brynjolfsson, S. F. et al. Long-Lived Plasma Cells in Mice and Men. Front Immunol 9, 2673, doi:10.3389/fimmu.2018.02673 (2018).
23 Atyeo, C. et al. Distinct Early Serological Signatures Track with SARS-CoV-2 Survival. Immunity, doi: 10.1016/j.immuni 0.2020.07.020 (2020).
24 Garimalla, S. et al. Differential transcriptome and development of human peripheral plasma cell subsets. JCI Insight 4, doi:10.1172/jci.insight.126732 (2019).
25 Lau, D. et al. Low CD21 expression defines a population of recent germinal center graduates primed for plasma cell differentiation. Sci Immunol 2, doi:10.1126/sciimmunol.aai8153 (2017).
26 Kaneko, N. et al. Loss of Bcl-6-Expressing T Follicular Helper Cells and Germinal Centers in COVID-19. Cell, doi:10.1016/j.cell.2020.08.025 (2020).
27 Dugan, H. L., Henry, C. & Wilson, P. C. Aging and influenza vaccine-induced immunity. Cell Immunol 348, 103998, doi:10.1016/j.cellimm.2019.103998 (2020).
28 Henry, C. et al. Influenza Virus Vaccination Elicits Poorly Adapted B Cell Responses in Elderly Individuals. Cell Host Microbe 25, 357-366 e356, doi: 10.1016/j.chom.2019.01.002 (2019).
29 Le Bert, N. et al. SARS-CoV-2-specific T cell immunity in cases of COVID-19 and SARS, and uninfected controls. Nature 584, 457-462, doi:10.1038/s41586-020-2550-z (2020).
30 Peng, Y. et al. Broad and strong memory CD4(+) and CD8(+) T cells induced by SARS-CoV-2 in UK convalescent individuals following COVID-19. Nat Immunol, doi:10.1038/s41590-020-0782-6 (2020).
31 Amanat, F. et al. A serological assay to detect SARS-CoV-2 seroconversion in humans. medRxiv, doi:10.1101/2020.03.17.20037713 (2020).
32 Stadlbauer, D. et al. SARS-CoV-2 Seroconversion in Humans: A Detailed Protocol for a Serological Assay, Antigen Production, and Test Setup. Curr Protoc Microbiol 57, el00, doi:10.1002/cpmc.100 (2020).
33 Nelson, C. A., Pekosz, A., Lee, C. A., Diamond, M. S. & Fremont, D. H. Structure and intracellular targeting of the SARS-coronavirus Orf7a accessory protein. Structure 13, 75-85, doi:10.1016/j.str.2004.10.010 (2005).
34 Nelson, C. A., Lee, C. A. & Fremont, D. H. Oxidative refolding from inclusion bodies. Methods Mol Biol 1140, 145-157, doi:10.1007/978-1-4939-0354-2_11 (2014).
35 Cao, J. et al. The single-cell transcriptional landscape of mammalian organogenesis. Nature 566, 496-502, doi:10.1038/s41586-019-0969-x (2019).
36 Qiu, X. et al. Reversed graph embedding resolves complex single-cell trajectories. Nat Methods 14, 979-982, doi:10.1038/nmeth.4402 (2017).
37 Stuart, T. et al. Comprehensive Integration of Single-Cell Data. Cell 177, 1888-1902 e1821, doi:10.1016/j.ce11.2019.05.031 (2019).
38 Guthmiller, J. J., Dugan, H. L., Neu, K. E., Lan, L. Y. & Wilson, P. C. An Efficient Method to Generate Monoclonal Antibodies from Human B Cells. Methods Mol Biol 1904, 109-145, doi:10.1007/978-1-4939-8958-4_5 (2019).
39 Andrews, S. F. et al. Immune history profoundly affects broadly protective B cell responses to influenza. Sci Transl Med 7, 316ra192, doi:10.1126/scitranslmed.aad0522 (2015).
40 Bunker, J. J. et al. Natural polyreactive IgA antibodies coat the intestinal microbiota. Science 358, doi:10.1126/science.aan6619 (2017).
41 Shlomchik, M. J., Aucoin, A. H., Pisetsky, D. S. & Weigert, M. G. Structure and function of anti-DNA autoantibodies derived from a single autoimmune mouse. Proc Natl Acad Sci USA 84, 9150-9154, doi:10.1073/pnas.84.24.9150 (1987).
42 Rydyznski Moderbacher, C. et al. Antigen-specific adaptive immunity to SARS-CoV-2 in acute COVID-19 and associations with age and disease severity. Cell doi:10.1016/j.ce11.2020.09.038 (2020).

### Example 2: Profiling B Cell Immunodominance after SARS-CoV-2 Infection Reveals Antibody Evolution to Non-Neutralizing Viral Targets

Dissecting the evolution of memory B cells (MBCs) against SARS-CoV-2 is critical for understanding antibody recall upon secondary exposure. Here, the inventors used single-cell sequencing to profile SARS-CoV-2-reactive B cells in 38 COVID-19 patients. Using oligo-tagged antigen baits, the inventors isolated B cells specific to the SARS-CoV-2 spike, nucleoprotein (NP), open reading frame 8 (ORFS), and endemic human coronavirus (HCoV) spike proteins. SARS-CoV-2 spike-specific cells were enriched in the memory compartment of acutely infected and convales-cent patients several months post symptom onset. With severe acute infection, substantial populations of endemic HCoV-reactive antibody-secreting cells were identified and possessed highly mutated variable genes, signifying preexisting immunity. Finally, MBCs exhibited pronounced maturation to NP and ORF8 overtime, especially in older patients. Monoclonal antibodies against these targets were non-neutralizing and non-protective in vivo. These findings reveal antibody adaptation to non-neutralizing intracellular antigens during infection, emphasizing the importance of vaccination for inducing neutralizing spike-specific MBCs.

Since the emergence of SARS-CoV-2 in December 2019, the World Health Organization has reported more than 160 million infections and 3 million deaths worldwide, with these statistics continuing to rise (World Health Organization, 2021). Faced with such persistence, the prospect of reinfection or infection with newly emerging variants warrants studies evaluating the generation of durable B cell memory upon infection.

Early in the pandemic, several independent groups identified that potently neutralizing antibodies are induced against the SARS-CoV-2 spike protein, the major antigenic glycoprotein of the virus (Chen et al., 2020; Lan et al., 2020; Robbiani et al., 2020; Wang et al., 2020; Yan et al., 2020; Yi et al., 2020). Since then, there has been a dedicated interest in the identification of durable memory B cells (MBCs) that provide protection from re-infection. The inventors' group and others have identified MBCs against the spike, nucleoprotein (NP), and open reading frame 8 (ORF8) proteins in convalescence, and some studies show that these populations persist several months after infection (Dan et al., 2021; Guthmiller et al., 2021; Hartley et al., 2020; Rodda et al., 2021). Beyond their longevity, spike-specific MBCs continue to adapt to SARS-CoV-2 up to 6 months post-infection, in a manner consistent with antigen persistence and ongoing germinal centers (GCs) (Gaebler et al., 2021; Sakharkar et al., 2021; Sokal et al., 2021).

Despite these advances, there is a lack of a clear understanding of MBC immunodominance and adaptation to distinct SARS-CoV-2 antigens over time and how this correlates with factors such as patient age and disease severity. Moreover, it remains to be determined whether MBCs to internal viral protein targets such as NP and ORF8 can provide protection from infection. Finally, the role of preexisting immunity to endemic human coronaviruses (HCoV) in shaping MBC responses to SARS-CoV-2 is poorly understood.

To address these knowledge gaps, the inventors characterized the SARS-CoV-2-specific B cell repertoire in 38 COVID-19 patients, both severe acute and convalescent, approximately 1.5-4.5 months post-symptom onset, using oligo-tagged antigen bait sorting and single-cell RNA sequencing (RNA-seq). Through this approach, the inventors provide a tool for evaluating human B cell subsets, immunodominance, and antibody adaptation to SARS-CoV-2 and have made a repository of more than 13,000 antibody sequences available to the SARS-CoV-2 research community.

These studies reveal that MBCs display substantial reactivity toward NP and ORF8 and continue to expand and adapt to these targets over time, particularly in older patients. Although SARS-CoV-2 receptor binding domain (RBD)-specific monoclonal antibodies (mAbs) were potently neutralizing and protective, the inventors showed that anti-NP and anti-ORF8 mAbs failed to neutralize and provide protection in vivo. Thus, preexisting MBC bias to non-neutralizing targets in SARS-CoV-2 could affect susceptibility to or severity of re-infection. Together, these findings highlight the importance of current SARS-CoV-2 vaccines, which are optimally formulated to induce protective MBC responses against the spike protein of SARS-CoV-2.

A. Results

Single-cell RNA-seq reveals substantial complexity among endemic HCoV- and SARS-CoV-2-specific B cells MBCs have potential to act as an early line of defense against viral infection, as they rapidly expand into antibody-secreting cells (ASCs) upon antigen re-encounter. To determine the landscape of MBC reactivity toward distinct SARS-CoV-2 and endemic HCoV spike viral targets, the inventors collected peripheral blood mononuclear cells (PBMCs) and serum between April and May 2020 from 10 severely infected acute subjects and 28 subjects upon recovery from SARS-CoV-2 viral infection (Tables S1-S4). In addition, 4 convalescent subjects returned approximately 4.5 months post-symptom onset for a second blood draw, with similar volumes of whole blood processed across time points. Severe acute infected samples were collected days 0, 1, 3, 5, and 14 before (day 0) and after receiving convalescent plasma therapy (Tables S3 and S4). All sampling time points were pooled from the same subjects for analysis because of small cell numbers.

To identify SARS-CoV-2-specific B cells, the inventors used the SARS-CoV-2 (SARS2) spike protein, spike RBD, NP, and ORF8 to generate probes for bait-sorting enriched B cells for subsequent single-cell RNA-seq analysis. This was done by conjugating distinct PE-streptavidin (SA)-oligos (BioLegend Total Seq) to individual biotinylated antigens (FIG. 10a). To control for non-specific B cell reactivity and B cells reactive to PE, and thus improve the specificity of sorting and downstream analysis, the inventors included an empty PE-SA-oligo, along with hantavirus PUUV, an irrelevant viral antigen control, on APC. Finally, to understand the impacts of preexisting immunity to endemic HCoV spike proteins, which share up to 30% amino acid identity with the SARS2 spike, the inventors included a cocktail of spike proteins from four coronavirus strains that cause mild upper respiratory infections in the vast majority of individuals: HCoV-229E, HCoV-NL63, HCoV-HKUL and HCoV-OC43, on one additional APC-SA-oligo.

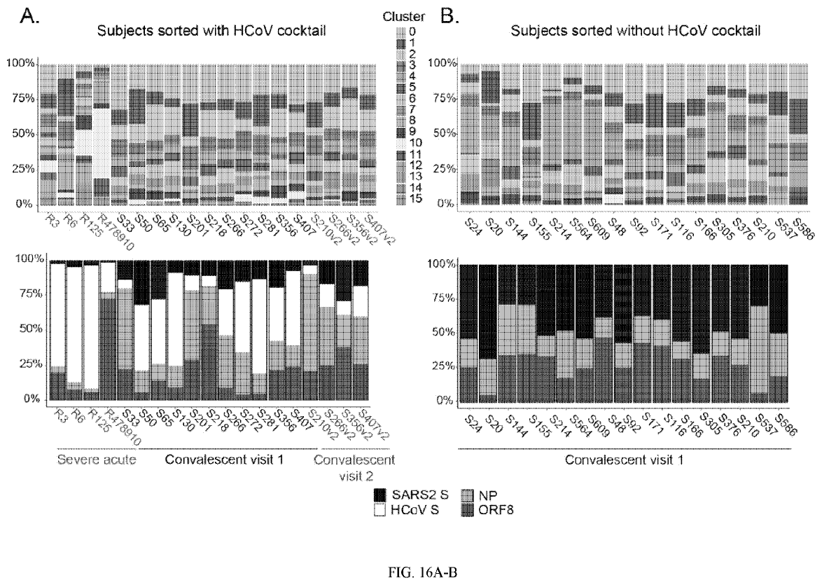

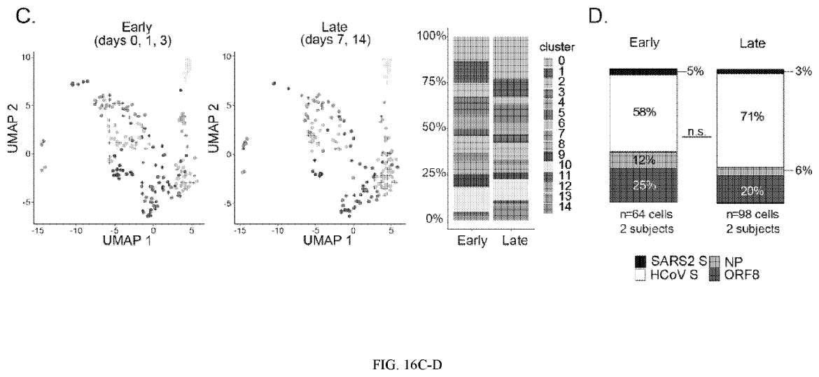

From a total of 38 subjects analyzed (including four matched follow-up visits ˜4.5 months post-symptom onset), the inventors detected small percentages (0.02%-1.25%) of SARS-CoV-2-reactive total CD19⁺ B cells, which were subsequently used to prepare 5° transcriptome, immunoglobulin (Ig) VDJ, and antigen-specific probe feature libraries for sequencing (FIG. 10a). The inventors sorted on total CD19+ B cells with elevated mean fluorescence intensity in order to capture highly specific cells regardless of naive-like or MBC origin, though a caveat of this approach may be the exclucion of lower affinity B cells. The inventors then integrated sequencing results from all 38 subjects using Seurat to remove batch effects and identified 16 transcriptionally distinct B cell clusters on the basis of expression profiles (FIG. 10b). Adopting the ROGUE scoring method, which compares how similar all transcriptomes within a cluster are to one another, the inventors determined that most clusters were highly pure, with the majority having a score over 0.9 (1.0 indicating 100% purity) (FIG. 10c; Liu et al., 2020). The inventors ensured that the feature libraries correlated with single-probe antigen-specific reactivity using a series of filtering steps to remove cells that were probe negative, multi-reactive and non-specific, empty PE-SA⁺, or Hanta-PUUV⁺. Because of the nature of this approach and the inability to clone antibodies from every B cell, it remains likely that a fraction of cells included in the analysis are non-specific and that a fraction of cells excluded either by gating or pre-filtering were actually specific. Therefore, the dataset represents only a subset of the total antigen-specific B cells induced by SARS-CoV-2. After all pre-filtering steps were complete, mapping only the cells that bound a single probe revealed that antigen-specific cells were enriched in distinct transcriptional clusters (FIGS. 10d-e), with considerable variation observed among individual subjects (FIGS. 16a-b). The inventors did not identify obvious differences in B cell subset distribution or antigen reactivity in B cells from severe acute subjects analyzed early (days 0, 1, and 3) or late (days 7 and 14 post-convalescent plasma therapy (FIGS. 16c-d). In summary, this method revealed substantial complexity in the B cell response to distinct coronavirus antigens, which the inventors then further dissected by subset.

1. The SARS-CoV-2-Specific B Cell Landscape is Defined by Naive-Like and MBC Subsets

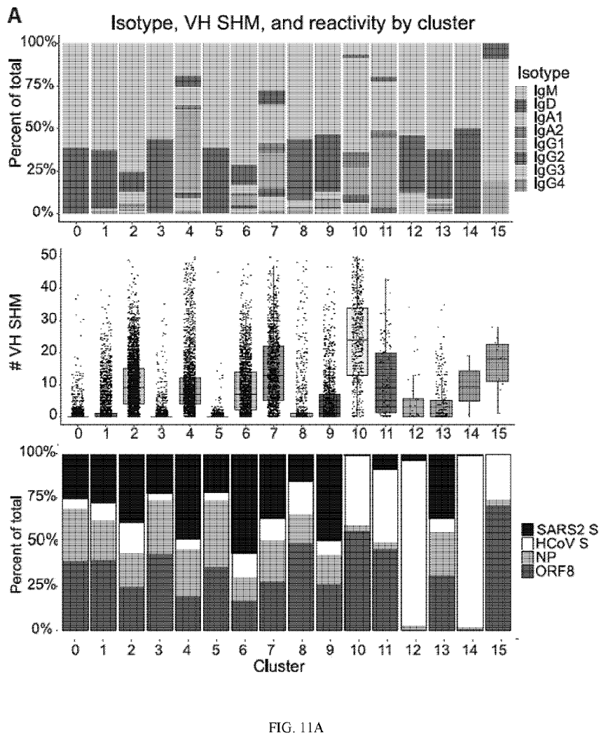

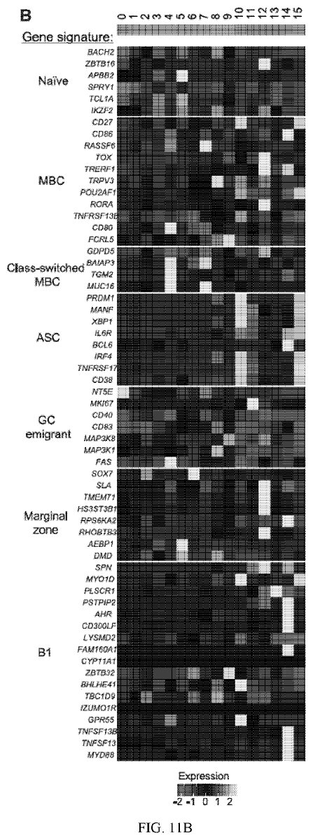

To discern the identity and specificity of each B cell cluster, the inventors analyzed Ig repertoire, variable heavy (VH) chain somatic hypermutation (SHM) rates, and differentially expressed genes. Different B cell clusters varied widely in their degree of class-switch recombination (CSR) and SHM, consistent with the presence of both naive-like and memory-like B cell subsets (FIG. 11a). Moreover, the inventors quantitatively identified that targeting of viral antigens was variable across clusters (FIG. 11a). To confirm B cell subset identities, the inventors curated lists of differentially expressed genes across clusters associated with naive B cells, MBCs, recent GC emigrant cells, ASCs, and innate-like B cells (including B1 B cells and marginal zone B cells) (FIG. 11b). Clusters 0, 1, 3, and 5 expressed Ig genes with little to no SHM or CSR and gene signatures associated with naive B cells, suggesting that these subsets were composed of naive-like B cells or very recently activated B cells (FIGS. 11a-b). In addition, clusters with patterns of higher CSR and SHM were further investigated for memory gene signatures. On the basis of expression of key genes (Tables S5 and S6) the inventors identified clusters 4, 6, 7, and 8 as MBCs; clusters 2, 9, and 13 as recent memory or GC emigrants; clusters 10, 11, and 15 as ASCs; and clusters 12 and 14 as innate-like in nature, though genes for these subsets are not well defined in humans (Figs a-b, bottom).

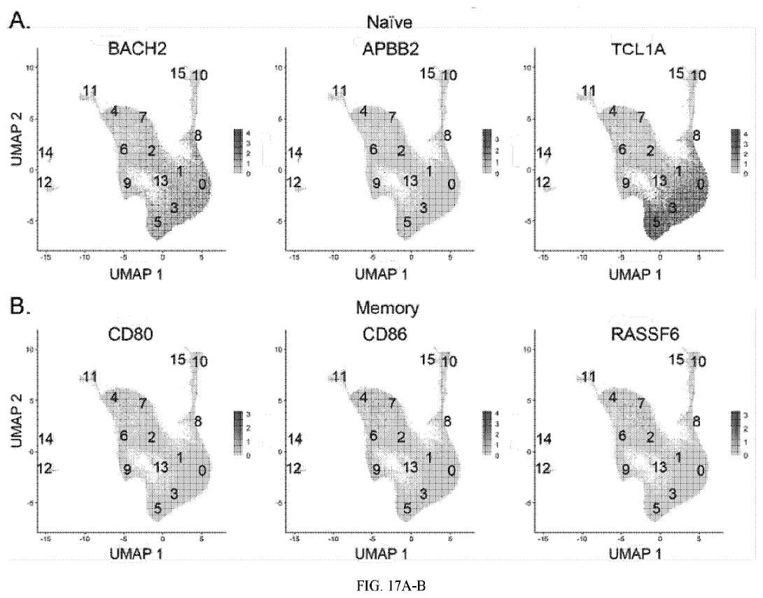

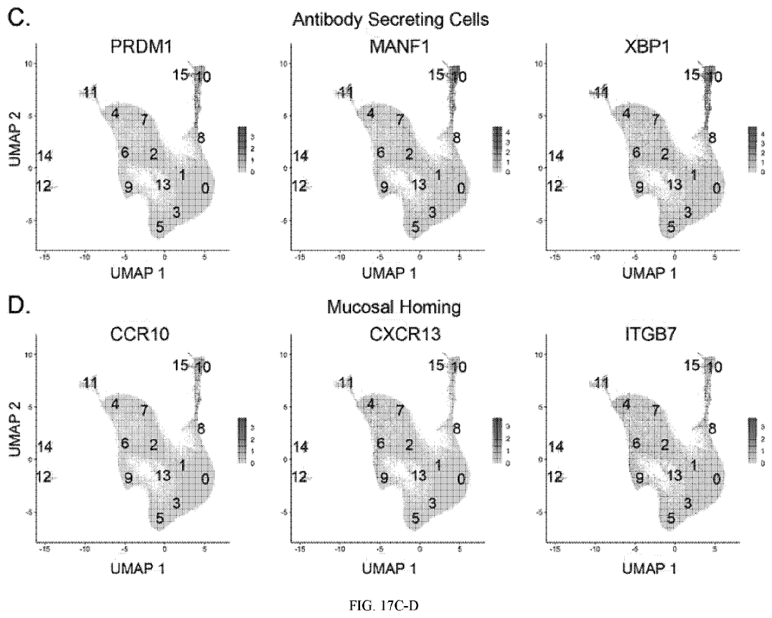

The inventors generated scores for each cluster and projected them onto UMAP, allowing us to visualize how closely associated clusters relate to one another on the basis of their B cell subset score (FIG. 11c). The inventors further visualized how cells clustered on the basis of identity by overlaying key gene signatures for MBCs, recent GC emigrants, and ASCs (Table S6). Some cells were outside of their home cluster, suggesting that they were in the course of differentiation and highlighting the plasticity of cells in an active immune response (FIGS. 17a-c). ASC clusters 10, 11, and 15 displayed a high degree of SHM, suggesting that they may derive from preexisting memory that was driven against endemic HCoV spike proteins (FIG. 11a). These clusters were also predominantly class-switched to IgA, an isotype most associated with mucosal immunity. To explore this possibility, the inventors mapped the expression of genes related to mucosal surface homing and found them to be highly expressed in ASC clusters, implying that memory to past HCoV infection generates a large plasmablast response during SARS-CoV-2 infection that re-circulates in the blood and should localize to mucosal surfaces (FIG. 17d). In conclusion, the inventors confirm that the landscape of B cell reactivity to SARS-CoV-2 and HCoV antigens is defined by distinct naive-like and MBC sub sets.

2. B Cell Immunodominance and Adaptability to SARS-CoV-2 and HCoVs Changes with Time after Infection

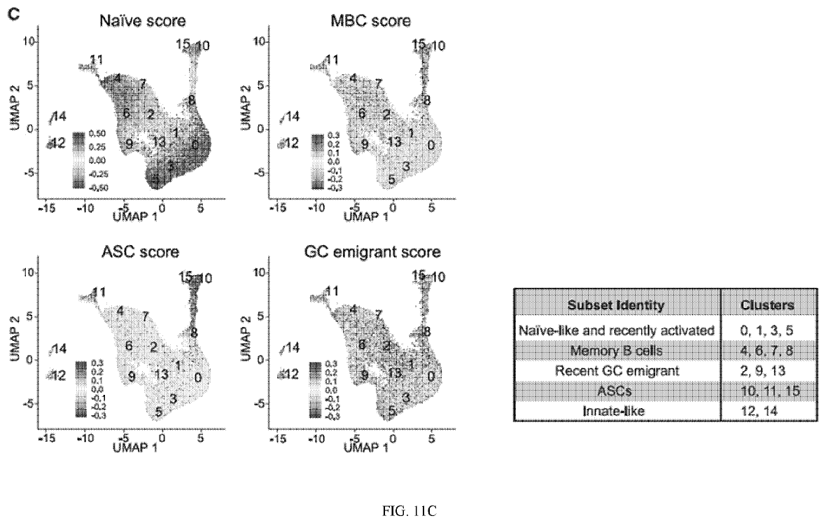

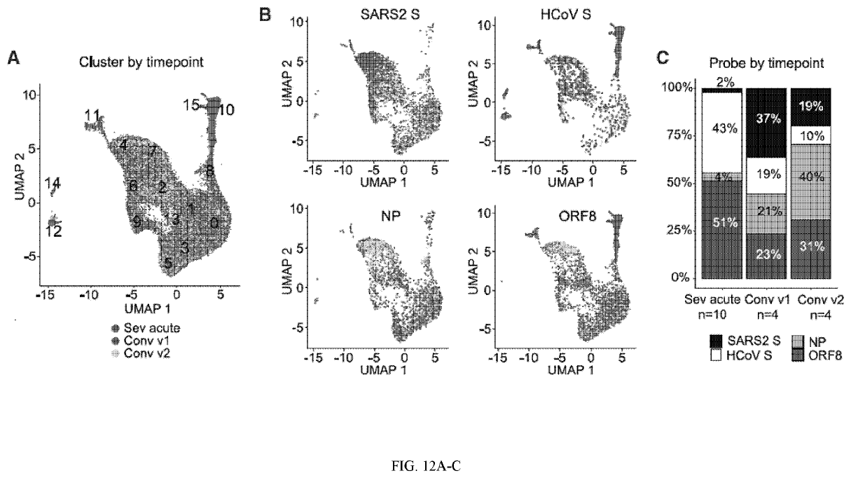

The kinetics and evolution of B cells against the spike and non-spike antigens are poorly understood. The inventors next investigated the dynamics of B cell subsets and their antigenic targets over time in severe acute subjects and convalescent subjects representing a range of disease severity. By color-coding cells belonging to the severe acute cohort (red), convalescent visit 1 (˜1.5 months post-symptom onset; blue), and convalescent visit 2 (˜4.5 months post-symptom onset; yellow) in the integrated UMAP, it became evident that distinct B cell subsets were enriched in different time points and cohorts. ASC clusters 10, 11, and 15 were derived predominantly from severe acute subjects (FIG. 12a). The two convalescent time points were composed largely of naive-like and MBC clusters, with convalescent visit 2 being the most enriched for canonical class-switched MBCs (clusters 4 and 7) (FIG. 12a). The severe acute cohort exhibited minimal targeting of the SARS2 spike protein and instead targeted HCoV spike and ORF8 (FIGS. 12b-c). As these ASCs were activated by SARS-CoV-2, it appeared that these were boosted MBCs with higher affinity for HCoV spikes and therefore displayed B cell receptors (BCRs) predominately loaded with HCoV spike probe when stained. In contrast, convalescent visit 1 was most enriched for SARS2 spike binding, which subsequently declined in percentage in convalescent visit 2, in which the frequency of B cells to NP and ORF8 was increased (FIGS. 12b-c).

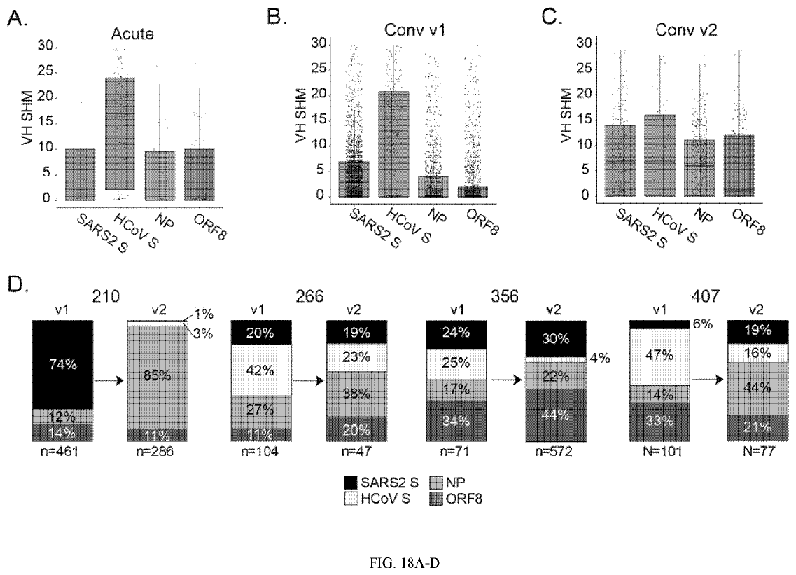

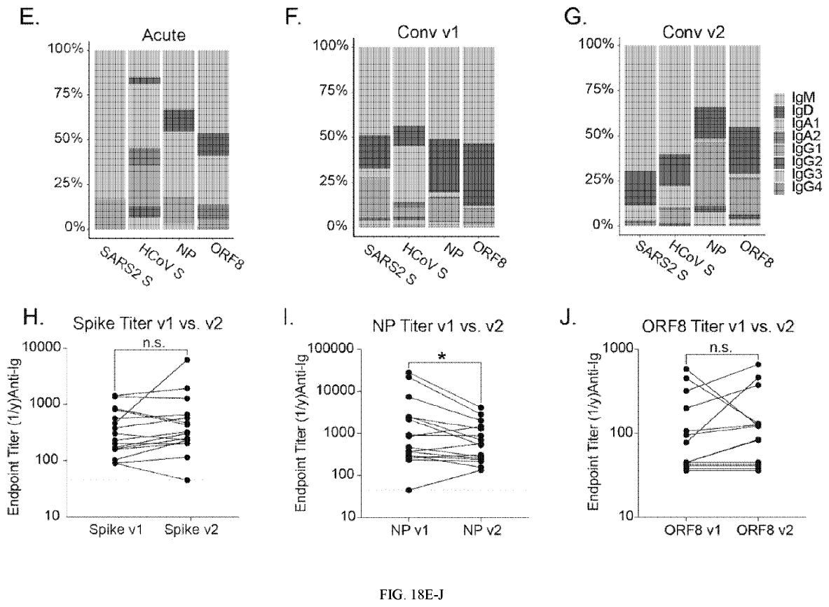

The dynamic change observed in antigen targeting over time led us to examine antigen reactivity within distinct B cell subsets for each cohort. For the severe acute cohort, B cells binding intracellular proteins were dominated by ASC clusters, whereas SARS2 spike-specific B cells were enriched in early memory and GC emigrant B cell clusters (FIG. 12d). As previously noted, HCoV spike-specific B cells were enriched in ASCs of the severe acute cohort, indicative of re-activation of preexisting immune memory. Consistent with this, HCoV spike-specific B cells were highly mutated in the acute cohort compared with SARS2 spike-, NP-, and ORF8-specific B cells (FIG. 18a).

Across the two convalescent visits, B cells reactive to ORF8 and NP were increased in percentage and absolute numbers relative to spike B cells (FIGS. 12e-g; total cell numbers indicated). Although the degree of SHM for all antigen-specific B cells was increased across study visits (FIG. 18h; FIGS. 18b-c), the B cells displaying the highest degree of SHM in convalescent visit 2 were majority NP-specific (FIGS. 12i-j). At the individual level, all four subjects displayed increases in the percentage of MBCs to NP across time points, and half of the subjects displayed modest increases to ORF8. The change in percentage for spike-specific B cells across visits was negligible for three of four subjects, with one subject displaying a substantial decrease (FIG. 18d, S210). Previous groups have identified that spike-specific MBCs increase over time (Dan et al., 2021; Rodda et al., 2021; Sokal et al., 2021), and the study is limited in that this analysis was performed in only four subjects. However, this data support the claim that there is MBC maturation to NP and, to a lesser extent, ORF8 over time.

Analyzing isotype frequencies by antigen specificity for each cohort revealed additional differences across time points. The majority of class-switched B cells were IgA in the severe acute cohort, regardless of antigen reactivity (FIG. 18e) In contrast, class switching to IgG1 was prominent for SARS2 spike-, NP-, and ORF8-reactive B cells in convalescent visit 1, while HCoV spike-reactive B cells remained largely IgA (FIG. 18f). Class-switched B cells specific to the SARS2 spike declined in conva visit 2, and IgG1 class-switched B cells to ORF8 and NP increased in proportion (FIG. 18g).

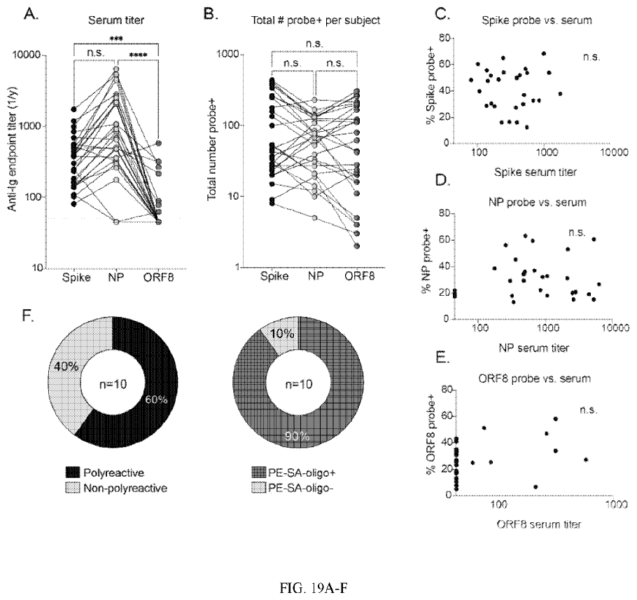

Finally, the inventors did not identify substantial differences in serum titer to distinct antigens across convalescent visit time points (FIGS. 18h-j). Similarly, reactivity patterns in serological titer and probe hit to distinct antigens in individual subjects did not appear to be correlated (FIGS. 19a-e). This may be related to differences in B cell affinity to three-dimensional probes in the bait-sorting assay versus ELISA or the fact that the cellular response is sampled at one snapshot in time (more than 1 month post-symptom onset), with serology reflective of antibody that has accumulated since initial infection.

Together, these results point to differences in B cell immunodominance and adaptability landscapes across severe acute and convalescent cohorts, independent of serum titer. For both the severe acute cohort and convalescent visit 1 time point, SARS2 spike-specific B cells were initially the most enriched cells in memory. However, NP- and ORF8-reactive MBCs increased in proportion and showed signs of adaptation over time.

3. SARS-CoV-2-Specific B Cells Display Unique Repertoire Features and Protective Ability

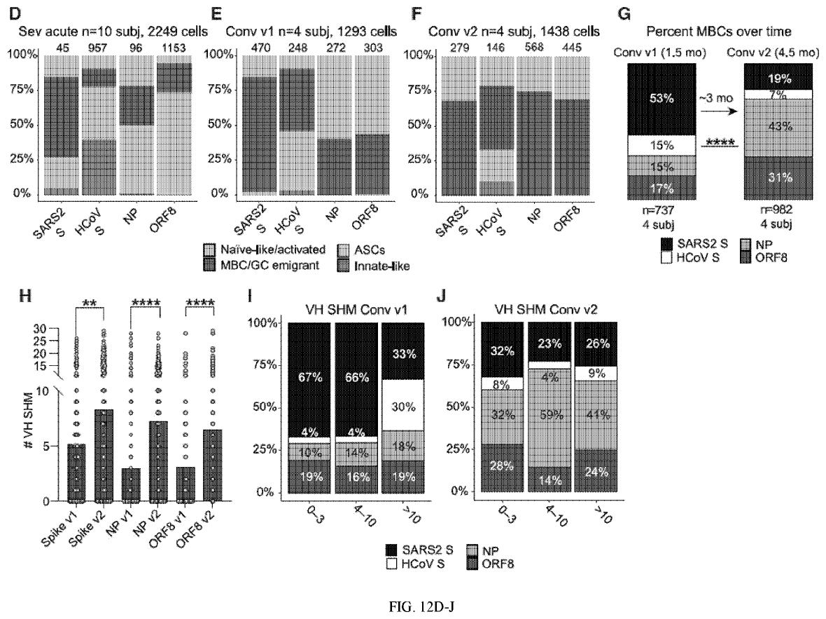

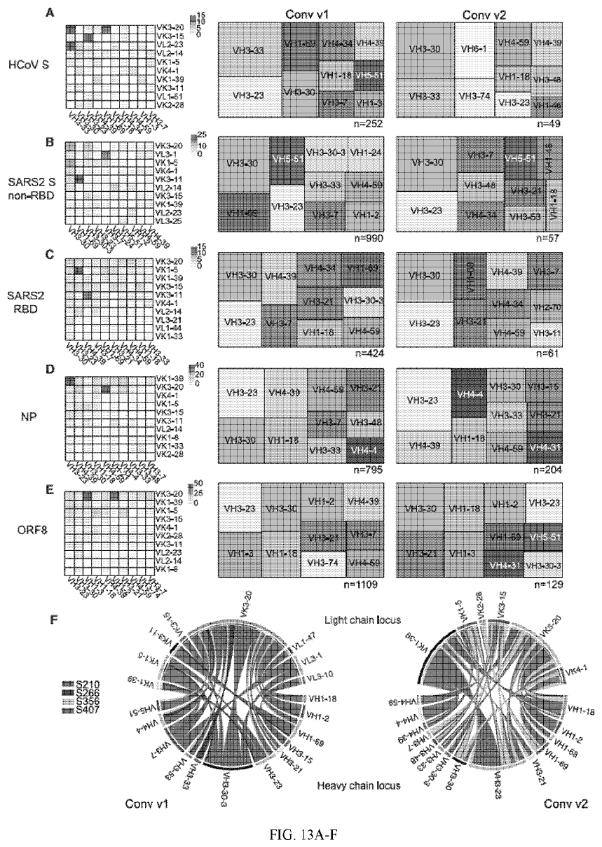

The identification of B cells against distinct antigens is typically associated with stereotypical VH and variable light-chain kappa (VK) or variable light-chain lambda (VL) gene usages. Immunodominant and neutralizing spike and RBD epitopes are of particular interest, as they represent key targets for vaccine-induced responses. To investigate whether antigen-specific B cells displayed enriched variable gene usages, the inventors analyzed VH and VK/VL pairs for B cells targeting HCoV spike, non-RBD spike epitopes, and RBD-specific epitopes. A B cell was considered non-RBD spike-specific if it bound full-length spike probe and not RBD probe, and a cell that bound both RBD and full-length spike was considered to be RBD-specific. Using this approach, the inventors found that B cells against HCoV spike, non-SARS2 RBD spike epitopes, and the SARS2 RBD were enriched for VH1-69 gene usage (FIGS. 13a-c). VH1-69 is commonly used by broadly neutralizing antibodies against the hemagglutinin stalk domain of influenza viruses, as well as the gp120 co-receptor binding site of HIV-1, because of its ability to bind conserved hydrophobic regions of viral envelope glycoproteins (Chen et al., 2019). VH1-69 usage by B cells that cross-react to SARS-CoV-2 and HCoV has also been indicated (Wec et al., 2020). However, VH1-69 usage for B cells targeting HCoV spike and SARS2 spike non-RBD epitopes was predominantly enriched in convalescent visit 1 subjects and not convalescent visit 2, suggesting that the repertoire may continue to evolve months after infection (FIGS. 13a-b, right). However, several VH gene usages were enriched in both convalescent visits, regardless of antigen specificity. For SARS2 spike non-RBD-specific B cells, VH3-7 and VH1-24 were also commonly used, which the inventors confirmed by characterizing cloned mAbs from the cohort (FIG. 13b; Table S7). Although NP-specific B cells used similar variable gene usages as RBD-specific B cells (FIG. 13d), ORF8-specific B cells were enriched for VH1-2 and VH1-3 paired with VK3-20, and enrichment for these VH genes persisted across both convalescent time points (FIG. 13e). Finally, by analyzing the frequency of the top ten heavy and light chain gene pairings (total antigen-specific cells) shared across subjects for both convalescent time points, the inventors observed variability among individual subjects and time points (FIG. 13f).

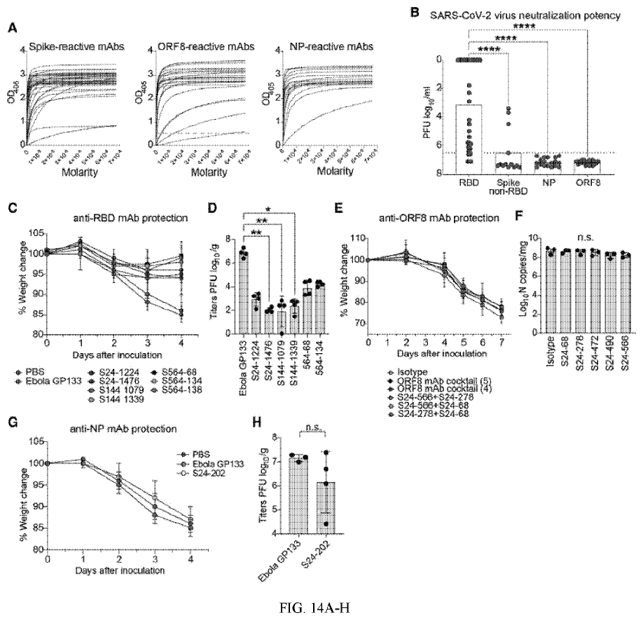

To better understand antigen-specific BCRs and how antigenic reactivity relates to immune effectiveness, the inventors next investigated the binding, neutralization potency, and in vivo protective ability of mAbs cloned from select BCRs. To do so, the inventors expressed nearly 100 mAbs against the SARS2 spike, NP, and ORF8 from convalescent subjects, representing a multi tude of clusters (Table S7). Cells from which to clone antibodies were chosen at random and were not chosen on the basis of specific sequence features. However, the inventors note that the results described herein may be affected by sampling bias, as only a small subset of antigen-specific mAbs were cloned. The inventors confirmed that cells designated as specific bound with moderate to high affinity to their corresponding antigens (FIG. 14a), and cells identified as multi-reactive exhibited features of polyreactivity or bound to PE (FIG. 19f). The inventors next tested the antibodies for viral neutralization using SARS-CoV-2/UW-001/Human/2020/Wisconsin virus plaque assays, where lower plaque-forming units (PFU) per milliliter equates to increased neutrali zation. Whereas 82% of mAbs to the RBD were neutralizing, including 42% exhibiting complete inhibition, only 23% of mAbs to spike regions outside of the RBD were neutralizing, and these showed relatively low potency (FIG. 14b). NP- and ORF8-specific mAbs were entirely non-neutralizing (FIG. 14b). Using animal models of SARS-CoV-2 infection, the inventors confirmed that anti-RBD antibodies were therapeutically protective in vivo, preventing weight loss and reducing lung viral titers relative to PBS control and an irrelevant Ebola anti-GP133 mAb (FIGS. 14c-d).

Although mAbs to NP and ORF8 were non-neutralizing in vitro, they might still provide protection in vivo, potentially through Fc-mediated pathways if the proteins were exposed on the virus or cell surface at appreciable levels. However, neither ORF8-reactive mAbs nor NP-reactive mAbs conferred protection from weight loss or viral infection in the lung in vivo (FIGS. 14e-h). Altogether, this data suggest that although B cells may continue to expand and evolve to intracellular antigens upon SARS-CoV-2 infection, B cell responses against these targets may not provide substantial protection from re-infection.

4. B Cell Immunodominance is Shaped by Age, Sex, and Disease Severity

Serum antibody titers to the spike and intracellular proteins are shown to correlate with age, sex, and SARS-CoV-2 severity (Atyeo et al., 2020; Guthmiller et al., 2021; Robbiani et al., 2020). The inventors therefore analyzed the distribution of B cell subsets and frequencies of B cells specific to the spike, NP, and ORF8 in convalescent subjects stratified by age, sex, and severity of disease. Disease severity was stratified into three categories: mild, moderate, and severe, on the basis of symptom duration and symptoms experienced (Table S1), as defined previously (Guthmiller et al., 2021).

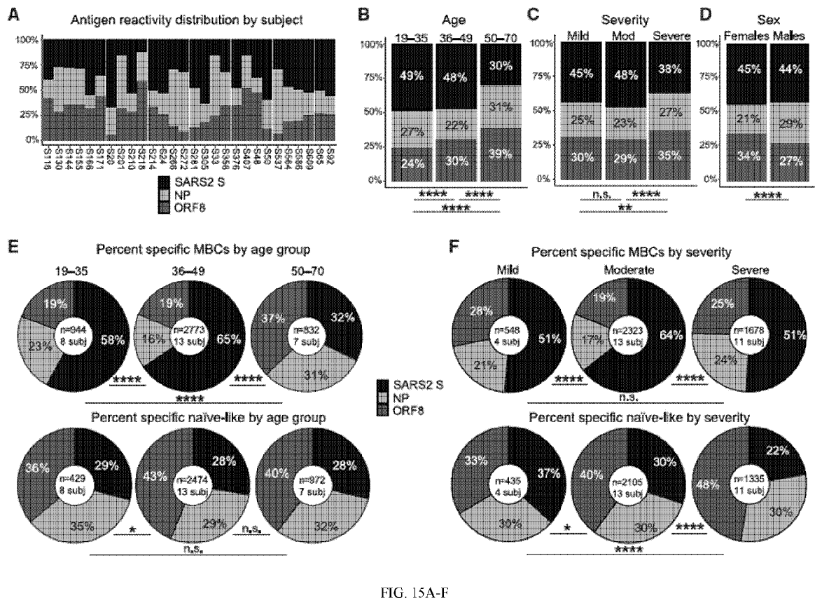

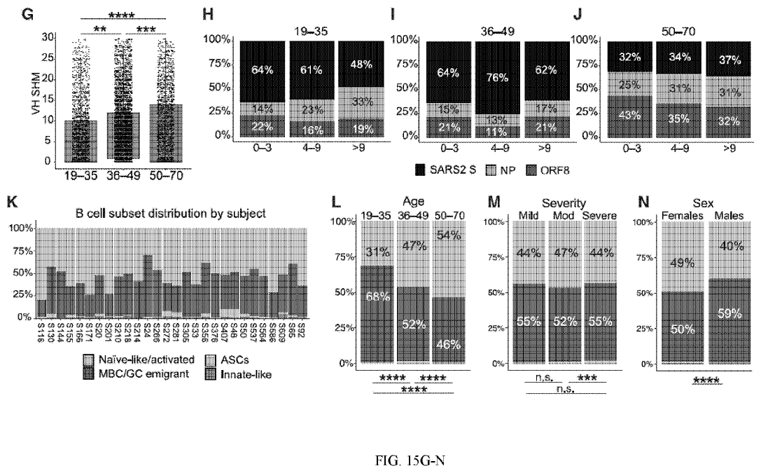

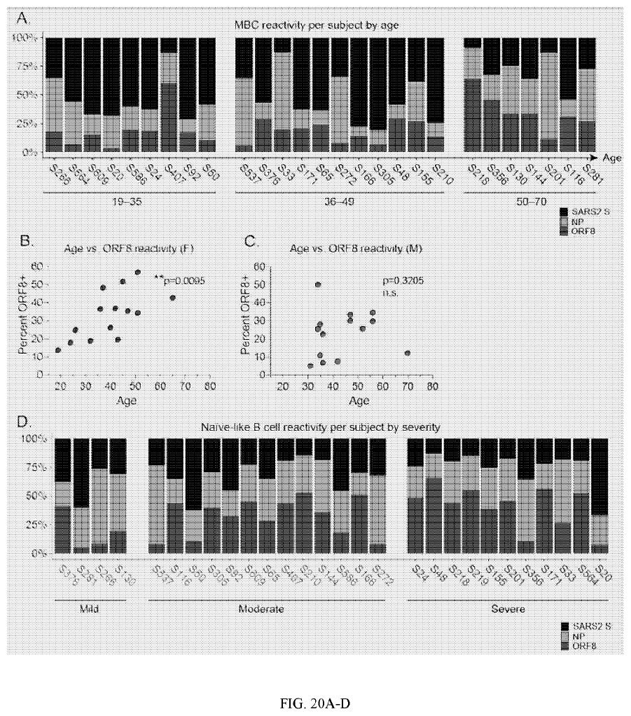

The inventors found that reactivity of total B cells toward different antigens varied widely by subject, likely reflecting host-intrinsic differences (FIG. 15a). With age, the inventors identified a decrease in the generation of spike-specific B cells and an increase in ORF8 and NP-specific B cells (FIG. 15b). Similarly, the percentage of total spike-specific B cells was reduced in subjects with more severe disease, whereas ORF8-specific B cells were increased (FIG. 15c). Last, the inventors identified that women had increased percentages of ORF8-reactive cells, whereas men showed slightly greater percentages of NP-reactive cells (Fig. To address whether differences in B cell reactivity with age and severity were associated with naive-like or MBC subsets, the inventors analyzed reactivity by subset. The inventors observed a substantial decrease in spike-specific MBCs and an increase in NP- and ORFS-reactive MBCs with age, while naive-like B cell subsets were more evenly distributed in reactivity across age groups (FIG. 15e; FIG. 20a). The inventors identified a significant correlation with age and the percentage of ORF8-reactive MBCs in women, but noting men (FIG. 20b-c). In contrast, the generation of specific MBCs was not different between mild and severe cases, though naive-like subsets targeting ORF8 were increased across mild, moderate, and severe disease (FIG. 15f; FIG. 20d).

Although B cell memory to the spike was decreased in older patients, the overall median number of VH SHMs for antigen-specific MBCs was increased relative to younger patients (FIG. 15g). However, whereas the majority of MBCs harboring the most mutations targeted the SARS2 spike in younger age groups (FIGS. 15h-i), mutated MBCs against NP and ORF8 were proportionately increased relative to the spike in older patients (FIG. 15j). Finally, the inventors observed variability in the percentages of MBCs and naive-like B cells across subjects (FIG. 15k), with older patients, patients with severe disease, and female patients generating reduced percentages of MBCs (FIGS. 15l-n). These findings point to older patients' exhibiting poorly adapted MBC responses to the spike, instead exhibiting increased targeting and adaptation to intracellular antigens. These data are analogous to B cell responses to influenza virus vaccination in the elderly and may be attributed to the effects of immunosenescence impairing the ability to form new memory over time (Dugan et al., 2020b; Henry et al., 2019). Alternatively, these findings may reflect potential effects of preexisting immunity on the boosting of NP-specific cross-reactive MBCs.

In summary, this study highlights the diversity of B cell subsets expanded upon novel infection with SARS-CoV-2. Using this approach, the inventors identified that B cells against the spike, ORFS, and NP differ in their ability to neutralize and derive from functionally distinct and differentially adapted B cell subsets; that MBC output over time shifts from the spike to intracellular antigens; and that targeting of these antigens is affected by age, sex, and disease severity.

B. Discussion

The COVID-19 pandemic continues to pose one of the greatest public health and policy challenges in modern history, and robust data on long-term immunity are critically needed to evaluate future decisions regarding COVID-19 responses. This approach combined three powerful aspects of B cell biology to address human immunity to SARS-CoV-2: B cell transcriptome, Ig sequencing, and recombinant mAb characterization. The inventors show that antibodies targeting key protective spike epitopes are enriched within MBC populations, but over time the MBC pool continues to adapt toward non-protective intracellular antigens, which could be a molecular hallmark of waning B-cell-mediated protection. This is further evidence that widespread vaccination, which only elicits a response to the spike, may be critical to end the pandemic.

Through this study, the inventors revealed that the landscape of antigen targeting and B cell subsets varied widely across severe acute subjects and convalescent subjects between 1.5 and 4.5 months post-symptom onset. Severe acute patients mounted a large ASC response toward HCoV spike and ORFS, derived largely from IgA ASC populations. The expansion of highly mutated plasmablasts to HCoV spike in severe acute patients suggests that the early response to SARS-CoV-2 in some patients may be dominated by an original antigen sin response, as plasma-blasts are often re-activated from preexisting memory (Dugan et al., 2020a). It remains unclear whether such responses worsen the severity of disease or reflect an inability to adapt to novel SARS2 spike epitopes. Alternatively, whether HCoV spike binding B cells adapt to the SARS2 spike and can provide protection is of interest for the potential generation of a universal coronavirus vaccine. Further investigation into the protection afforded by cross-reactive antibodies is warranted, as previous studies have identified cross-reactive HCoV and SARS1 binding antibodies can neutralize SARS-CoV-2 (Ng et al., 2020; Wec et al., 2020). Vaccine-induced responses to the spike will also be shaped by preexisting immunity and should be investigated.

Although SARS2 spike-specific B cells from the convalescent cohort were enriched in memory, the inventors also identified MBCs and ASCs to HCoV spike, which waned 4.5 months after infection. This later time point coincided with an increase in overall numbers and percentage of ORF8- and NP-specific MBCs, which displayed a marked increase in SHIM. This phenotype was pronounced in older patients, who exhibited reduced MBC targeting of the spike. Patients who were older, were female, and had more severe disease showed increased B cell targeting of ORF8, and older patients tended to generate more memory to intracellular proteins over time. The inventors identified B cells targeting these intracellular proteins as exclusively non-neutralizing and non-protective. Mechanistically, these observations may be explained by reduced adaptability of B cells or increased reliance on CD4 T cell help for B cell activation, which have been observed in aged individuals upon viral infections and are dysregulated in aged patients (Dugan et al., 2020b; Henry et al., 2019) Furthermore, T cell responses to SARS-CoV-2 intracellular proteins are prevalent in convalescent COVID-19 patients (Grifoni et al., 2020; Le Bert et al., 2020; Peng et al., 2020). The shift in memory output during convalescence may also reflect the massive difference in pro tein availability, with each virion producing only dozens of spikes but thousands of intracellular proteins (Grifoni et al., 2020; Lu et al., 2021; Yao et al., 2020).

Finally, the identification of multiple distinct antigen-specific subsets of naive-like, innate-like B cells, MBCs, and ASCs illustrates the complexity of the B cell response to SARS-CoV-2, revealing an important feature of the immune response against any novel pathogen. More research is warranted to determine whether the expansion of particular antigen-specific B cell subsets directly affects susceptibility and disease severity and, conversely, whether age or disease severity shape memory formation. Addressing these questions will be critical for understanding the disease course, determining correlates of protection, and developing vaccines capable of protecting against SARS-CoV-2 and emerging variants.

C. Experimental Model and Subject Details

1. Human Materials

All studies were performed with the approval of the University of Chicago institutional review board IRB20-0523 and University of Chicago, University of Wisconsin-Madison, and Washington University in St. Louis institutional biosafety committees. Informed consent was obtained after the research applications and possible consequences of the studies were disclosed to study subjects. This clinical trial was registered at ClinicalTrials.gov with identifier NCT04340050, and clinical information for patients included in the study is detailed in Tables S1-S3. Convalescent leukoreduction filter donors were 18 years of age or older, eligible to donate blood as per standard University of Chicago Medicine Blood Donation Center guidelines, had a documented COVID-19 polymerase chain reaction (PCR) positive test, and complete resolution of symptoms at least 28 days prior to donation. Severe acute infected blood donors were 18 years of age or older and blood was collected per standard University of Chicago Medical Center guidelines. Subjects had a documented COVID-19 polymerase chain reaction (PCR) positive test, were hospitalized, and had been scheduled to receive an infusion of convalescent donor plasma. Four blood draws were collected both before and after plasma infusion, at days 0, 1, 3, and 14. PBMCs were collected from leukoreduction filters or blood draws within 2 hours post-collection and, if applicable, flushed from the filters using sterile 1× Phosphate-Buffered Saline (PBS, GIBCO) supplemented with 0.2% Bovine Serum Albumin (BSA, Sigma). Lymphocytes were purified by Lymphoprep Ficoll gradient (Thermo Fisher) and contaminating red blood cells were lysed by ACK buffer (Thermo Fisher). Cells were frozen in Fetal Bovine Serum (FBS, GIBCO) with 10% Dimethyl sulfoxide (DMSO, Sigma) prior to downstream analysis. On the day of sorting, B cells were enriched using the human pan B cell EasySep™ enrichment kit (STEMCELL).

D. Method Details

1. Recombinant Proteins and Probe Generation

SARS-CoV-2 and Hanta PUUV proteins were obtained from the Krammer laboratory at Mt. Sinai, the Joachimiak laboratory at Argonne, and the Fremont laboratory at Washington University. pCAGGS expression constructs for the spike protein, spike RBD, and hanta PUUV were obtained from the Krammer lab at Mt. Sinai and produced in house in Expi293F suspension cells (Thermo Fisher). Sequences for the spike and RBD proteins as well as details regarding their expression and purification have been previously described (Amanat et al., 2020; Stadlbauer et al., 2020). Proteins were biotinylated for 2 hours on ice using EZ-Link Sulfo-NHS-Biotin, No-Weigh Format (Thermo Fisher) according to the manufacturer's instructions, unless previously Avi-tagged and biotinylated (ORF8 protein, Fremont laboratory). Truncated cDNAs encoding the Ig-like domains of ORF8 were inserted into the bacterial expression vector pET-21(a) in frame with a biotin ligase recognition sequence at the c-terminus (GLNDIFEAQKIEWHE). Soluble recombinant proteins were produced as described previously (Nelson et al., 2005). In brief, inclusion body proteins were washed, denatured, reduced, and then renatured by rapid dilution following standard methods (Nelson et al., 2014). The refolding buffer consisted of 400 mM arginine, 100 mM Tris-HCl, 2 mM EDTA, 200 mM ABESF, 5 mM reduced glutathione, and 500 mM oxidized glutathione at a final pH of 8.3. After 24 hr, the soluble-refolded protein was collected over a 10 kDa ultrafiltration disc (EMD Millipore, PLGC07610) in a stirred cell concentrator and subjected to chromatography on a HiLoad 26/60 Superdex S75 column (GE Healthcare). Site specific biotinylation with BirA enzyme was done following the manufacture's protocol (Avidity) except that the reaction buffer consisted of 100 mM Tri s-HCl (pH7.5) 150 mM NaCl, with 5 mM MgCl2 in place of 0.5 M Bicine at pH 8.3. Unreacted biotin was removed by passage through a 7K MWCO desalting column (Zeba spin, Thermo Fisher). Full-length SARS-CoV-2 NP was cloned into pET21a with a hexahistidine tag and expressed using BL21(DE3)-RIL E. coli in Terrific Broth (bioWORLD) Following overnight induction at 25° C., cells were lysed in 20 mM Tris-HCl pH 8.5, 1 M NaCl, 5 mM b-mercaptopethanol, and 5 mM imidazole for nickel-affinity purification and size exclusion chromatography. Endemic HCoV spike proteins (HCoV-229E, HCoV-NL63, HCoV-HKU1, and HCoV-OC43) were purchased from Sino Biological. Biotinylated proteins were then conjugated to Biolegend TotalSeq PE streptavidin (PE-SA), APC streptavidin (APC-SA), or non-fluorescent streptavidin (NF-SA) oligos at a molar ratio of antigen to PE-SA, APC-SA, or NF-SA. The amount of antigen was chosen based on a fixed amount of 0.5 mg PE-SA, APC-SA, or NF-SA and diluted in a final volume of 10 mL. PE-SA, APC-SA, or NF-SA was then added gradually to 10 mL biotinylated proteins times on ice, 1 mL PE-SA, APC-SA, or NF-SA (0.1 mg/ml stock) every 20 minutes for a total of 5 mL (0.5 mg) PE-SA, APC-SA, or NF-SA. The reaction was then quenched with 5 mL 4 mM Pierce biotin (Thermo Fisher) for 30 minutes for a total probe volume of 20 mL. Probes were then used immediately for staining.

2. Antigen-Specific B Cell Sorting

PBMCs were thawed and B cells were enriched using EasySep™ pan B cell magnetic enrichment kit (STEMCELL). B cells were stained with a panel containing CD19 PE-Cy7 (Biolegend), IgM APC (Southern Biotech), CD27 BV605 (Biolegend), CD38 BB515 (BD Biosciences), and CD3 BV510 (BD Biosciences). B cells were stained with surface stain master mix and each COVID-19 antigen probe for 30 minutes on ice in 1×PBS supplemented with 0.2% BSA and 2 mM Pierce Biotin. Cells were stained with probe at a 1:100 dilution (NP, ORFS, RBD, PUUV, empty PE-SA) or 1:200 dilution (spike, endemic HCoV spikes). Cells were subsequently washed with 1×PBS 0.2% BSA and stained with Live/Dead BV510 (Thermo Fisher) in 1×PBS for 15 minutes. Cells were washed again and re-suspended at a maximum of 4 million cells/mL in 1×PBS supplemented with 0.2% BSA and 2 mM Pierce Biotin for downstream cell sorting using the MACSQuantTyto cartridge sorting platform (Miltenyi). Cells that were viable/CD19⁺/antigen-PE⁺ or viable/CD19⁺/antigen-APC were sorted as probe positive. The PE⁺ and APC⁺ gates were drawn by use of FMO controls. Cells were then collected from the cartridge sorting chamber and used for downstream 10× Genomics analysis.

3. 10× Genomics Library Construction

VDJ, 5°, and probe feature libraries were prepared using the 10× Chromium System (10× Genomics). The Chromium Single Cell 5° Library and Gel Bead v2 Kit, Human B Cell V(D)J Enrichment Kit, and Feature Barcode Library Kit were used. All steps were followed as listed in the manufacturer's instructions. Specifically, user guide CG000186 Rev D was used. Severe acute infected samples were pooled post-sort and hashtagged (Biolegend), and run as a single sample, to account for low cell numbers. Final libraries were pooled and sequenced using the NextSeq550 (Illumina) with 26 cycles apportioned for read 1, 8 cycles for the i7 index, and 134 cycles for read 2.

4. Computational Analyses for Single Cell Sequencing Data

The inventors adopted Cell Ranger (version 3.0.2) for raw sequencing processing, including 5° gene expression analysis, antigen probe analysis, and immunoprofiling analysis of B cells. Based on Cell Ranger output, the inventors performed downstream analysis using Seurat (version 3.9.9, an R package, for transcriptome, cell surface protein and antigen probe analysis) and IgBlast (version 1.15, for immunoglobulin gene analysis). For transcriptome analysis, Seurat was used for cell quality control, data normalization, data scaling, dimension reduction (both linear and non-linear), clustering, differential expression analysis, batch effects correction, and data visualization. Unwanted cells were removed according to the number of detectable genes (number of genes <200 or >2500 were removed) and percentage of mitochondrial genes for each cell. A soft threshold of percentage of mitochondrial genes was set to the 95^{th} percentile of the current dataset distribution, and the soft threshold was subject to a sealing point of 10% as the maximum threshold in the case of particularly poor cell quality. Transcriptome data were normalized by a log-transform function with a scaling factor of whereas cell surface protein and antigen probe were normalized by a centered log-ratio (CLR) normalization. The inventors used variable genes in principal component analysis (PCA) and used the top 15 principal components (PCs) in non-linear dimension reduction and clustering. High-quality cells were then clustered by Louvain algorithm implemented in Seurat under the resolution of 0.6. Differentially expressed genes for each cell cluster were identified using a Wilcoxon rank-sum test implemented in Seurat. Batch effects correction analysis was performed using an Anchor method implemented in Seurat to remove batch effects across different datasets. All computational analyses were performed in R (version 3.6.3).

5. ROGUE Scoring

To assess the quality of B cell subsets identified in this study the inventors used ROGUE scoring, an entropy-based metric for assessing the purity of single cell populations, adapted from a previous study (Liu et al., 2020). The expression entropy for each gene was calculated using “SE_fun” from the “ROGUE” package (version 1.0). Based on the expression entropy, the ROGUE score for each cluster was calculated using the “rogue” function from the same package with parameters “platform” set to “UMI” and “span” set to

6. Antigen Probe Reactivity Assignment

Antigen probe signals were normalized by a centered log-ratio transformation individually for each subject. All B cells were sub-sequently clustered into multiple probe-specific groups according to their normalized probe signals. By investigating all normalized antigen-probe binding signals, the inventors arbitrarily set a threshold equal to 1 for all normalized probe signals to distinguish probe binding cells as “positive” or “negative.” Cells that were negative to all probes were clustered into the “negative” group; those positive to only one probe were clustered into corresponding probe-specific groups; and those that were positive to multiple probes were further investigated. Only cells whose top hit probe value was at least two-fold greater than their second hit probe value were clustered into the top hit probe-specific group; others were clustered into the “multi-reactive” group that indicates non-specific cells. To account for the inclusion of endemic HCoV spike protein reactivity in some samples, cells positive to both SARS2 spike and endemic spike were further clustered into a group the inventors assigned as “spike cross-reactive” in the code. For samples in which the inventors included separate SARS2 spike and RBD oligo tags, the inventors placed cells positive to both SARS2 spike and SARS2 RBD into the “spike” group.

7. Gene Module Scoring

Scores for B cell-genotype-related gene modules (e.g., MBC score, naive score, ASC score, and GC emigrant score) were calculated using the “AddModuleScore” function from the Seurat package (Stuart et al., 2019). The naive score was calculated based on the genes BACH2, ZBTB16, APBB2, SPRY1, TCL1A, and IKZF2; the MBC score was calculated based on the genes CD27, CD86, RASSF6, TOX, TRERF 1, TRPV3, POU2AF, RORA, TNFRSF13B, CD80, and FCRL5; the ASC score was calculated based on genes PRDM1, MANF, XBP1, IL6R, BCL6, IRF4, TNFRSF17, and CD38; and the GC emigrant score was calculated based on genes NT5E, MK167, CD40, CD83, TNFRSF13B, MAP3K8, MAP3K1, and FAS.

8. Selection of Antibodies for mAb Synthesis

Representative antibodies from each subject were chosen for synthesis by choosing random samplings of B cells that bound to a given antigen probe with higher intensity relative to all other probes. B cells with varying ranges of probe-binding intensities were chosen for confirmation by ELISAs. In addition, B cells representing select public clonal expansions were also chosen for cloning. B cells binding to all probes in a polyreactive manner were also chosen and validated for polyreactivity by polyreactivity ELISA (see methods below).

9. Monoclonal Antibody Generation

Immunoglobulin heavy and light chain genes were obtained by 10× Genomics VDJ sequencing analysis and monoclonal antibodies (mAbs) were synthesized by Integrated DNA Technologies. Cloning, transfection, and mAb purification have been previously described (Guthmiller et al., 2019). Briefly, sequences were cloned into human IgG1 expression vectors using Gibson assembly, and heavy and light genes were co-transfected into 293T cells (Thermo Fisher). Secreted mAbs were then purified from the supernatant using protein A agarose beads (Thermo Fisher).

10. Enzyme-Linked Immunosorbent Assay (ELISA)

High-protein binding microtiter plates (Costar) were coated with recombinant SARS-CoV-2 proteins at 2 mg/ml in 1×PBS overnight at 4° C. Plates were washed the next morning with 1×PBS 0.05% Tween and blocked with 1×PBS containing 20% fetal bovine serum (FBS) for 1 hour at 37° C. Antibodies were then serially diluted 1:3 starting at 10 mg/ml and incubated for 1 hour at 37° C. Horseradish peroxidase (HRP)-conjugated goat anti-human IgG antibody diluted 1:1000 (Jackson Immuno Research) was used to detect binding of mAbs, and plates were subsequently developed with Super Aquablue ELISA substrate (eBiosciences). Absorbance was measured at 405 nm on a microplate spectrophotometer (BioRad). To standardize the assays, control antibodies with known binding characteristics were included on each plate and the plates were developed when the absorbance of the control reached 3.0 OD₄₀₅ units. All experiments were performed in duplicate 2-3 times.

11. Polyreactivity ELISA

Polyreactivity ELISAs were performed as previously described (Andrews et al., 2015; Bunker et al., 2017; Guthmiller et al., 2020). High-protein binding microtiter plates (Costar) were coated with 10 mg/ml calf thymus dsDNA (Thermo Fisher), 2 mg/ml Salmonella enterica serovar Typhimurium flagellin (Invitrogen), 5 mg/ml human insulin (Sigma-Aldrich), 10 mg/ml KLH (Invitrogen), and 10 mg/ml Escherichia coli LPS (Sigma-Aldrich) in 1×PBS. Plates were coated with 10 mg/ml cardiolipin in 100% ethanol and allowed to dry overnight. Plates were washed with water and blocked with 1×PBS/0.05% Tween/1 mM EDTA. MAbs were diluted 1 mg/ml in PBS and serially diluted 4-fold, and added to plates for 1.5 hours. Goat anti-human IgG-HRP (Jackson Immunoresearch) was diluted 1:2000 in PBS/0.05% Tween/1 mM EDTA and added to plates for 1 hour. Plates were developed with Super Aquablue ELISA substrate (eBioscience) until the positive control mAb, 3H9 (Shlomchik et al., 1987), reached an OD₄₀₅ of 3. All experiments were performed in duplicate.

12. Neutralization Assay

The SARS-CoV-2/UW-001/Human/2020/Wisconsin (UW-001) virus was isolated from a mild case in February 2020 and used to assess neutralization ability of monoclonal antibodies (mAbs). Virus (˜500 plaque-forming units) was incubated with each mAb at a final concentration of 10 mg/ml. After a 30-minute incubation at 37° C., the virus/antibody mixture was used to inoculate Vero E6/TMPRSS2 cells seeded a day prior at 200,000 cells per well of a TC12 plate. After 30 minutes at 37° C., cells were washed three times to remove any unbound virus, and media containing antibody (10 mg/ml) was added back to each well. Two days after inoculation, cell culture supernatant was harvested and stored at −80° C. until needed. A non-relevant Ebola virus GP mAb and PBS were used as controls.

To determine the amount of virus in the cell culture supernatant of each well, a standard plaque-forming assay was performed. Confluent Vero E6/TMPRSS2 cells in a TC12 plate were infected with supernatant (undiluted, 10-fold dilutions from 10⁻¹ to 10⁻⁵) for 30 minutes at 37° C. After the incubation, cells were washed three times to remove unbound virus and 1.0% methylcellulose media was added over the cells. After an incubation of three days at 37° C., the cells were fixed and stained with crystal violet solution in order to count the number plaques at each dilution and determine virus concentration given as plaque-forming units (PFU)/ml.

13. In Vivo Protection Assays

To evaluate the efficacy of RBD and NP monoclonal antibodies (mAbs) in vivo, groups of 4-5-week-old female Syrian golden hamsters (four animals in each group) were infected with SARS-CoV-2 at a dose of 10³ PFU by intranasal inoculation. One day later, the hamsters were treated by intraperitoneal injection with one of the mAbs at 5 mg/kg. Control groups of hamsters were injected with either sterile PBS or a non-relevant mAb (Ebola glycoprotein 133/3.16). Weights were recorded daily. Four days after the infection, nasal turbinate and lung samples were collected to determine viral loads in these tissues by standard plaque assay on Vero E6/TMPRRSS2 cells. All animal studies were conducted under BSL-3 containment with an approved protocol reviewed by the Institutional Animal Care and Use Committee at the University of Wisconsin.

Studies with mice were carried out in accordance with the recommendations in the Guide for the Care and Use of Laboratory Animals of the National Institutes of Health. The protocols were approved by the Institutional Animal Care and Use Committee at the Washington University School of Medicine (assurance number A3381-01). Virus inoculations were performed under anesthesia that was induced and maintained with ketamine hydrochloride and xylazine, and all efforts were made to minimize animal suffering. To evaluate the efficacy of ORF8 mAbs in vivo, eight-week-old heterozygous female K18-hACE c57BL/6J mice (strain: 2B6.Cg-Tg(K18-ACE2)2Prlmn/J) received 200 mg of each indicated mAb by intraperitoneal injection one day prior to intranasal inoculation with 10³ PFU of SARS-CoV-2 (n-CoV/USA_WA1/2020 strain). Weight change was monitored daily and lungs were harvested at 7 days post-infection. Viral RNA levels in lung homogenates were determined by qRT-PCR quantifying N gene copy number and compared to a standard curve as described previously (Winkler et al., 2020).

14. Quantification and Statistical Analysis

All statistical analysis was performed using Prism software (Graphpad Version 9.0) or R. Chi-square tests were corrected for multiple comparisons using post hoc Chi-square test. Sample sizes (n) are indicated in corresponding figures or figure legends. The number of biological repeats for experiments and specific tests for statistical significance used are indicated in the figure legends. P values less than or equal to 0.05 were considered significant. *p<0.05, **p<0.01, ***p<0.001, ****p<0.0001.

### E. Tables

TABLE S1
Convalescent patient information, Related to FIGS. 10-15.

| | | | | | | | Symptom |
|---|---|---|---|---|---|---|---|
| | | | | | | Duration | start to |
| Subject | | | Reported | Severity | Severity | symptoms | donation |
| ID | Age | Sex | symptoms* | Score | Category | (days) | (days) |
| 24 | 34 | M | Fatigue, cough, SOB, | 19 | Severe | 12 | 41 |
| | | | SC, fever, headache, | | | | |
| | | | BAP, diarrhea, LOS, | | | | |
| | | | LOT | | | | |
| 20 | 31 | M | Fatigue, cough, SOB, | 29 | Critical | 19 | 48 |
| | | | SC, fever, headache, | | | | |
| | | | BAP, LOS, LOT | | | | |
| 564 | 24 | F | Fatigue, cough, SOB, | 24 | Severe | 32 | 60 |
| | | | SC, ST, fever, | | | | |
| | | | headache, BAP, | | | | |
| | | | diarrhea, LOS, LOT | | | | |
| 144 | 56 | M | Fatigue, cough, SC, | 17 | Moderate | 23 | 54 |
| | | | ST, headache, BAP, | | | | |
| | | | LOS | | | | |
| 214 | 47 | M | Fatigue, cough, SOB, | 20 | Severe | 24 | 59 |
| | | | SC, ST, headache, | | | | |
| | | | BAP, LOS, LOT | | | | |
| 171 | 37 | F | Fatigue, cough, SOB, | 21 | Severe | 16 | 44 |
| | | | SC, fever, headache, | | | | |
| | | | BAP, diarrhea, LOS, | | | | |
| | | | LOT | | | | |
| 92 | 35 | M | Fatigue, cough, SC, | 16 | Moderate | 16 | 47 |
| | | | ST, fever, headache, | | | | |
| | | | BAP | | | | |
| 48 | 45 | F | Fatigue, cough, SOB, | 19 | Severe | 8 | 40 |
| | | | SC, ST, fever, | | | | |
| | | | headache, AP, | | | | |
| | | | diarrhea, LOS, LOT | | | | |
| 537 | 36 | M | Fatigue, cough, | 13 | Moderate | 14 | 59 |
| | | | fever, BAP | | | | |
| 586 | 32 | F | Fatigue, cough, SOB, | 18 | Moderate | 17 | 61 |
| | | | SC, headache, BAP, | | | | |
| | | | AP. diarrhea | | | | |
| 376 | 36 | F | Diarrhea, LOS, LOT | 8 | Mild | 7 | 48 |
| 305 | 43 | F | Fatigue, cough, SC. | 14 | Moderate | 4 | 47 |
| | | | ST, fever, headache, | | | | |
| | | | BAP, LOS, LOT | | | | |
| 116 | 65 | F | Cough, SOB, fever, | 13 | Moderate | 18 | 49 |
| | | | LOS, LOT | | | | |
| 166 | 42 | F | Fatigue, cough, SOB, | 18 | Moderate | 17 | 55 |
| | | | SC, fever, headache, | | | | |
| | | | BAP, diarrhea, LOS, | | | | |
| | | | LOT | | | | |
| 155 | 47 | F | Fatigue, cough, SOB, | 20 | Severe | 29 | 64 |
| | | | ST, fever, BAP, LOS, | | | | |
| | | | LOT | | | | |
| 609 | 26 | F | Fatigue, SOB, ST, | 16 | Moderate | 7 | 57 |
| | | | fever, headache, | | | | |
| | | | BAP. LOS, LOT | | | | |
| 130 | 52 | M | Fatigue, SC, | 10 | Mild | 7 | 35 |
| | | | headache, LOS, LOT | | | | |
| 281 | 70 | M | Cough, fever, BAP | 9 | Mild | 7 | 48 |
| 272 | 42 | M | Fatigue, cough, SOB, | 18 | Moderate | 14 | 43 |
| | | | fever, headache, | | | | |
| | | | BAP, LOS, LOT | | | | |
| 50 | 35 | M | Fatigue, SC, fever, | 13 | Moderate | 10 | 40 |
| | | | BAP, LOS, LOT | | | | |
| 65 | 40 | F | Fatigue, SC, fever, | 16 | Moderate | 13 | 47 |
| | | | headache, BAP, | | | | |
| | | | diarrhea, LOS, LOT | | | | |
| 33 | 36 | M | Fatigue, cough, SOB, | 22 | Severe | 14 | 48 |
| | | | SC, fever, headache, | | | | |
| | | | BAP, AP, diarrhea, | | | | |
| | | | LOS. LOT | | | | |
| 201 | 56 | M | Fatigue, cough, SOB, | 20 | Severe | 18 | 58 |
| | | | SC, ST, fever, | | | | |
| | | | headache, BAP, | | | | |
| | | | LOS, LOT | | | | |
| 218 | 51 | F | Fatigue, cough, SOB, | 19 | Severe | 19 | 48 |
| | | | fever, headache, | | | | |
| | | | BAP, AP, diarrhea | | | | |
| 266 | 19 | F | Fatigue, cough, SC, | 9 | Mild | 4 | 32, 137 |
| | | | headache, BAP | | | | V2* |
| 356 | 51 | F | Fatigue, cough, ST, | 20 | Severe | 14 | 43, 137 |
| | | | fever, headache, | | | | V2* |
| | | | BAP, AP, diarrhea, | | | | |
| | | | LOS, LOT | | | | |
| 407 | 34 | M | Fatigue, cough, SC, | 16 | Moderate | 11 | 43, 131 |
| | | | fever, BAP, AP, | | | | V2* |
| | | | diarrhea, LOS, LOT | | | | |
| 210 | 47 | M | Fatigue, cough, SOB, | 16 | Moderate | 7 | 41, 125 |
| | | | fever, headache, | | | | V2* |
| | | | BAP, LOS, LOT | | | | |

*SOB = shortness of breath; SC = sinus congestion; ST = sore throat; BAP = body aches and pain; AP = abdominal pain; LOS = loss of smell; LOT = loss of taste. Starred symptom start to donation values indicate the value for follow-up visit donation (V2). Severity scoring method has been described previously (Guthmiller et al., 2021).

TABLE S2
Distribution of clinical parameters for convalescent patients
included in the study, Related to FIGS. 10-15

| | |
|---|---|
| Median Age | 41 |
| Mean Age | 42 |
| Mode Age | 47 |
| Range Age | 19-70 |
| Number of Males | 14 |
| Number of Females | 14 |
| Median Duration of Symptoms (days) | 14 |
| Mean Duration of Symptoms (days) | 14 |
| Mode Duration of Symptoms (days) | 7 |
| Range Duration of Symptoms (days) | 4-32 |
| Median symptom start to donation (days) | 48 |
| Mean symptom start to donation (days) | 49 |
| Mode symptom start to donation (days) | 48 |
| Range symptom start to donation (days) | 32-64 |

TABLE S3
Severe acute patient information, Related to FIGS. 10-12.

| | | | | Symptom | | |
|---|---|---|---|---|---|---|
| | | | | start to first | | |
| Subject | | | Reported | donation | | |
| ID | Age | Sex | symptoms* | (days) | Co-morbidities* | COVID treatment |
| R1 | 57 | M | Fever, cough, | 3 | HTN, DM, NAFLD | Tocilizumab, |
| | | | nausea | | | mechanical |
| | | | | | | ventilation |
| R2 | 61 | M | Cough, | 16 | None | Hydroxychloroquine, |
| | | | weakness, | | | nasal cannula |
| | | | hiccups, altered | | | |
| | | | mental status | | | |
| R3 | 51 | F | Fever, cough, | 21 | HTN, DM, PE, asthma | Remdesivir, |
| | | | dyspnea | | | tocilizumab, |
| | | | | | | venovenous ECMO* |
| R4 | 70 | F | Fever, altered | 2 | HTN, Alzheimer's | Nasal cannula |
| | | | mental status | | disease | |
| R5 | 66 | F | Altered mental | 9 | HTN, PE/DVT, recent | Nasal cannula |
| | | | status, dyspnea | | hospitalization for | |
| | | | | | orthopedic procedure | |
| R6 | 59 | M | Fever, chills, | 20 | HTN, DM | Remdesivir, |
| | | | decreased | | | tocilizumab, |
| | | | appetite, | | | Venovenous ECMO |
| | | | dizziness | | | |
| R7 | 57 | M | Dyspnea | 9 | HTN, Myelodysplastic | Tocilizumab, |
| | | | | | syndrome s/p stem | anakinra, nasal |
| | | | | | cell transplant | cannula |
| R8 | 30 | M | Fever, chills, | 13 | Cystic fibrosis s/p | Room air |
| | | | fatigue, LOT* | | bilateral lung | |
| | | | | | transplant, DM | |
| R9 | 78 | M | Fever, cough | 14 | HTN, prostate cancer | High-flow nasal |
| | | | | | | cannula |
| R10 | 86 | F | Dyspnea, | 6 | ESRD on HD, stroke, | Nasal cannula |
| | | | abdominal pain | | PVD s/p AKA, DM, | |
| | | | | | PE/DVT, CHF | |

*LOT: Loss of taste; ECMO: Extracorporeal membrane oxygenation.
*AKA, above the knee amputation; CHF, congestive heart failure; DM, diabetes mellitus; DVT, deep venous thrombosis; ESRD, end-stage renal disease; HTN, hypertension; NAFLD, non-alcoholic fatty liver disease; PE, pulmonary embolism; PVD, peripheral vascular disease

TABLE S4
Distribution of clinical parameters for severe acute patients
included in the study, Related to FIGS. 10-12.

| | |
|---|---|
| Median Age | 60 |
| Mean Age | 61.5 |
| Mode Age | 57 |
| Range Age | 30-86 |
| Number of Males | 6 |
| Number of Females | 4 |
| Median symptom start to first donation* (days) | 11 |
| Mean symptom start to first donation* (days) | 11 |
| Mode symptom start to first donation* (days) | 9 |
| Median symptom start to last donation* (days) | 25 |
| Mean symptom start to last donation* (days) | 25 |
| Mode symptom start to last donation* (days) | 23 |

*Samples were collected day 0 (pre-plasma transfusion), 1, 3, 5, and 14 post-plasma transfusion and all time points per subject were pooled for analysis due to low cell numbers. See methods for additional details.

TABLE S6
Key genes used in the identification of B cell subsets, Related to FIG. 11.

| Gene | B Cell Subset | Rationale | Citation |
|---|---|---|---|
| BACH2 | Naïve | Promotes B cell | (Itoh-Nakadai et al., |
| | | development, maintains | 2014) |
| | | mature B cells | |
| ZBTB16 | Naïve | Downregulated in | (Moroney et al., |
| | | memory compared to | 2020) |
| | | naïve | |
| APBB2 | Naïve | Foxp1 target important for | (Patzelt et al., 2018); |
| | | mature FO B cell survival | The Human Protein |
| | | | Atlas (Uhlen et al., |
| | | | 2015) |
| SPRY1 | Naïve | Proliferation inhibitor, | (Frank et al., 2009) |
| | | differentially expressed | The Human Protein |
| | | (DE) between naïve and | Atlas (Uhlen et al., |
| | | memory | 2015) |
| TCL1A | Naïve | DE between B cell pop. | (Said et al., 2001) |
| | | High in Naïve, low in GC, | |
| | | absent in memory and | |
| | | ASC | |
| IKZF2 | Naïve | DE between memory and | (Moroney et al., |
| | | naïve, higher in naïve | 2020) |
| CD27 | Memory | Classic memory marker | (Palm and Henry, |
| | | | 2019) |
| CD86 | Memory | DE between memory and | (Axelsson et al., |
| | | naïve, higher in memory | 2020) |
| RASSF6 | Memory | Increased in memory | (Moroney et al., |
| | | | 2020) |
| TOX | Memory | Increased in memory | (Moroney et al., |
| | | | 2020) |
| TRERF1 | Memory | Increased in memory | (Moroney et al., |
| | | | 2020) |
| TRPV3 | Memory | Increased in memory | (Moroney et al., |
| | | | 2020) |
| POU2AF1 | Memory | B cell-specific TF | (Zhao et al., 2008) |
| RORA | Memory | Increased in memory | (Moroney et al., |
| | | | 2020) |
| TNFRSF13B | Memory | BAFF-binding receptor | (Muller-Winkler et |
| | | expressed in memory and | al., 2021) |
| | | ASC | |
| CD80 | Memory | High affinity memory | (Palm and Henry. |
| | | marker | 2019) |
| FCLR5 | Memory | Atypical memory marker | (Kim et al., 2019) |
| GDPD5 | Class-switched | Highest in class-switched | The Human Protein |
| | Memory | memory B cells | Atlas (Uhlen et al., |
| | | | 2015) |
| BAIAP3 | Class-switched | DE in switched memory, | (Moroney et al., |
| | Memory | ion channel Ca²⁺ flux | 2020) |
| TGM2 | Class-switched | DE in switched memory, | (Moroney et al., |
| | Memory | Ca²⁺ signal transduction | 2020) |
| MUC16 | Class-switched | DE in class-switched | (Moroney et al., |
| | Memory | memory, membrane | 2020) |
| | | adhesion | |
| PRDM1 | ASC | Lineage-defining TF | (Lightman et al., |
| | | | 2019) |
| MANF | ASC | ER stress | (Lightman et al., |
| | | | 2019) |
| XBP1 | ASC | Unfolded protein | (Lightman et al., |
| | | response | 2019) |
| IL6R | ASC | Receptor for IL6, | (Dienz et al., 2009) |
| | | promotes PC fate and | |
| | | mAb production | |
| BCL6 | ASC | Drops in GCs to promote | (Palm and Henry, |
| | | PC fate | 2019) |
| IRF4 | ASC | Rises as BCL6 drops to | (Palm and Henry, |
| | | promote PC fate | 2019) |
| TNFSR17 | ASC | Genetic KOs experience | (Lightman et al., |
| | | sig PC reduction | 2019) |
| CD38 | ASC | Classic PC marker | (Lightman et al., |
| | | | 2019) |
| NT5E | GC emigrant/ | Important for class-switch | (Schena et al., 2013) |
| | recent MBC | | |
| MKI67 | GC emigrant/ | Proliferation marker | (Scholzen and |
| | recent MBC | | Gerdes, 2000) |
| CD40 | GC emigrant/ | Required for memory | (Basso et al., 2004) |
| | recent MBC | formation | |
| CD83 | GC emigrant/ | GC composition | (Krzyzak et al., |
| | recent MBC | | 2016) |
| MAP3K8 | GC emigrant/ | DE during GC reaction | (Wohner et al., 2016) |
| | recent MBC | | |
| MAP3K1 | GC emigrant/ | Required for CD40 | (Gallagher et al., |
| | recent MBC | signaling | 2007) |
| FAS | GC emigrant/ | DE during GC reaction | (Smith et al., 1995) |
| | recent MBC | | |
| Marginal Zone | Marginal Zone | DE in MZBs | (Descatoire et al., |
| genes | B cells | | 2014) |
| SPN | B1 B Cells | Classic B1 marker | (Rothstein et al., |
| | | | 2013) |
| MYO1D | B1 B Cells | DE in B1s | (Macias-Garcia et |
| | | | al., 2016) |
| PLSCR1 | B1 B Cells | Expressed in natural | Cordero et al |
| | | ASCs | |
| PSTPIP2 | B1 B Cell | DE during activation | (Ochiai et al., 2020) |
| AHR | B1 B Cell | Highest expression in B1 | (Villa et al., 2017) |
| CD300LF | B1 B Cell | DE in B1s | (Macias-Garcia et |
| | | | al., 2016) |
| LYSMD2- | B1 B Cell | DE in mouse B1s | (Mabbott and Gray, |
| GPR55 | | | 2014) |
| IZUMO1R | B1 B Cell | DE in B1s | (Macias-Garcia et |
| | | | al., 2016) |
| TNFSF13B- | Innate-like B | Highly expressed in MZB | (Smulski and Eibel, |
| MYD88 | cells | and B1 | 2018) |

### F. References

The following references, to the extent that they provide exemplary procedural or other details supplementary to those set forth herein, are specifically incorporated herein by reference.
1. Amanat, F., Stadlbauer, D., Strohmeier, S., Nguyen, T. H. O., Chromikova, V., McMahon, M., Jiang, K., Asthagiri Arunkumar, G., Jurczyszak, D., Polanco, J., et al. (2020). A serological assay to detect SARS-CoV-2 seroconversion in humans. medRxiv.
2. Andrews, S. F., Huang, Y., Kaur, K., Popova, L. I., Ho, I. Y., Pauli, N. T., Henry Dunand, C. J., Taylor, W. M., Lim, S., Huang, M., et al. (2015). Immune history profoundly affects broadly protective B cell responses to influenza. Sci. Transl. Med. 7, 316ra192.
3. Atyeo, C., Fischinger, S., Zohar, T., Slein, M. D., Burke, J., Loos, C., McCulloch, D. J., Newman, K. L., Wolf, C., Yu, J., et al. (2020). Distinct early serological signatures track with SARS-CoV-2 survival. Immunity 53, 524-532.e4.
4. Axelsson, S., Magnuson, A., Lange, A., Alshamari, A., Hornquist, E. H., and Hultgren, (2020). A combination of the activation marker CD86 and the immune checkpoint marker B and T lymphocyte attenuator (BTLA) indicates a putative permissive activation state of B cell subtypes in healthy blood donors independent of age and sex. BMC Immunol. 21, 14.
5. Basso, K., Klein, U., Niu, H., Stolovitzky, G. A., Tu, Y., Califano, A., Cattoretti, G., and Dalla-Favera, R. (2004). Tracking CD40 signaling during germinal center development. Blood 104, 4088-4096.
6. Bunker, J. J., Erickson, S. A., Flynn, T. M., Henry, C., Koval, J. C., Meisel, M., Jabri, B., Antonopoulos, D. A., Wilson, P. C., and Bendelac, A. (2017). Natural polyreactive IgA antibodies coat the intestinal microbiota. Science 358, eaan6619.
7. Chen, F., Tzarum, N., Wilson, I. A., and Law, M. (2019). VH1-69 antiviral broadly neutralizing antibodies: genetics, structures, and relevance to rational vaccine design. Curr. Opin. Virol. 34, 149-159.
8. Chen, X., Li, R., Pan, Z., Qian, C., Yang, Y., You, R., Zhao, J., Liu, P., Gao, L., Li, Z., et al. (2020). Human monoclonal antibodies block the binding of SARS-CoV-2 spike protein to angiotensin converting enzyme 2 receptor. Cell. Mol. Immunol. 17, 647-649.
9. Dan, J. M., Mateus, J., Kato, Y., Hastie, K. M., Yu, E. D., Faliti, C. E., Grifoni, A., Ramirez, S. I., Haupt, S., Frazier, A., et al. (2021). Immunological memory to SARS-CoV-2 assessed for up to 8 months after infection. Science 371, eabf4063.
10. Descatoire, M., Weller, S., Irtan, S., Sarnacki, S., Feuillard, J., Storck, S., Guiochon-Mantel, A., Bouligand, J., Morali, A., Cohen, J., et al. (2014). Identification of a human splenic marginal zone B cell precursor with NOTCH2-dependent differentiation properties. J. Exp. Med. 211, 987-1000.
11. Dienz, O., Eaton, S. M., Bond, J. P., Neveu, W., Moquin, D., Noubade, R., Briso, E. M., Charland, C., Leonard, W. J., Ciliberto, G., et al. (2009). The induction of antibody production by IL-6 is indirectly mediated by IL-21 produced by CD4+ T cells. J. Exp. Med. 206, 69-78.
12. Dugan, H. L., Guthmiller, J. J., Arevalo, P., Huang, M., Chen, Y. Q., Neu, K. E., Henry, C., Zheng, N. Y., Lan, L. Y., Tepora, M. E., et al. (2020a). Preexisting immunity shapes distinct antibody landscapes after influenza virus infection and vaccination in humans. Sci. Transl. Med. 12, eabd3601.
13. Dugan, H. L., Henry, C., and Wilson, P. C. (2020b). Aging and influenza vaccine-induced immunity. Cell. Immunol. 348, 103998.
14. Frank, M. J., Dawson, D. W., Bensinger, S. J., Hong, J. S., Knosp, W. M., Xu, L., Balatoni, C. E., Allen, E. L., Shen, R. R., Bar-Sagi, D., et al. (2009). Expression of sprouty2 inhibits B-cell proliferation and is epigenetically silenced in mouse and human B-cell lymphomas. Blood 113, 2478-2487.
15. Gaebler, C., Wang, Z., Lorenzi, J. C. C., Muecksch, F., Finkin, S., Tokuyama, M., Cho, A., Jankovic, M., Schaefer-Babajew, D., Oliveira, T. Y., et al. (2021). Evolution of antibody immunity to SARS-CoV-2. Nature 591, 639-644.
16. Gallagher, E., Enzler, T., Matsuzawa, A., Anzelon-Mills, A., Otero, D., Holzer, R., Janssen, E., Gao, M., and Karin, M. (2007). Kinase MEKK1 is required for CD40-dependent activation of the kinases Jnk and p38, germinal center formation, B cell proliferation and antibody production. Nat. Immunol. 8, 57-63.
17. Grifoni, A., Weiskopf, D., Ramirez, S. I., Mateus, J., Dan, J. M., Moderbacher, C. R., Rawlings, S. A., Sutherland, A., Premkumar, L., Jadi, R. S., et al (2020). Targets of T cell responses to SARS-CoV-2 coronavirus in humans with COVED-19 disease and unexposed individuals. Cell 181, 1489-1501.e15.
18. Guthmiller, J. J., Dugan, H. L., Neu, K. E., Lan, L. Y., and Wilson, P. C. (2019). An efficient method to generate monoclonal antibodies from human B cells. Methods Mol. Biol. 1904, 109-145.
19. Guthmiller, J. J., Lan, L. Y., ndez-Quintero, M. L., Han, J., Utset, H. A., Bitar, D. J., Hamel, N.J., Stovicek, O., Li, L., Tepora, M., et al. (2020). Polyreactive broadly neutralizing B cells are selected to provide defense against pandemic threat influenza viruses. Immunity 53, 1230-1244.e5.
20. Guthmiller, J. J., Stovicek, O., Wang, J., Changrob, S., Li, L., Halfmann, P., Zheng, N. Y., Utset, H., Stamper, C. T., Dugan, H. L., et al. (2021). SARS-CoV-2 infection severity is linked to superior humoral immunity against the spike. MBio 12, e02940-20.
21. Hartley, G. E., Edwards, E. S. J., Aui, P. M., Varese, N., Stojanovic, S.,
22. McMahon, J., Peleg, A. Y., Boo, I., Drummer, H. E., Hogarth, P. M., et al. (2020). Rapid generation of durable B cell memory to SARS-CoV-2 spike and nucleocapsid proteins in COVID-19 and convalescence. Sci. Immunol. 5, eabf8891.
23. Henry, C., Zheng, N.Y., Huang, M., Cabanov, A., Rojas, K. T., Kaur, K., Andrews, S. F., Palm, A. E., Chen, Y. Q., Li, Y., et al. (2019). Influenza virus vaccination elicits poorly adapted B cell responses in elderly individuals. Cell Host Microbe 25, 357-366.e6.
24. Itoh-Nakadai, A., Hikota, R., Muto, A., Kometani, K., Watanabe-Matsui, M., Sato, Y., Kobayashi, M., Nakamura, A., Miura, Y., Yano, Y., et al. (2014). The transcription repressors Bach2 and Bach1 promote B cell development by repressing the myeloid program. Nat. Immunol. 15, 1171-1180.
25. Kim, C. C., Baccarella, A. M., Bayat, A., Pepper, M., and Fontana, M. F. (2019). FCRL5+ memory B cells exhibit robust recall responses. Cell Rep. 27, 1446— 1460.e4.
26. Krzyzak, L., Seitz, C., Urbat, A., Hutzler, S., Ostalecki, C., Gla€sner, J., Hiergeist, A., Gessner, A., Winkler, T. H., Steinkasserer, A., and Nitschke, L. (2016). CD83 modulates B cell activation and germinal center responses. J. Immunol. 196, 3581-3594.
27. Lan, J., Ge, J., Yu, J., Shan, S., Zhou, H., Fan, S., Zhang, Q., Shi, X., Wang, Q., Zhang, L., and Wang, X. (2020). Structure of the SARS-CoV-2 spike receptor-binding domain bound to the ACE2 receptor. Nature 581, 215-220.
28. Le Bert, N., Tan, A. T., Kunasegaran, K., Tham, C. Y. L., Hafezi, M., Chia, A.,
29. Chng, M. H. Y., Lin, M., Tan, N., Linster, M., et al. (2020). SARS-CoV-2-specific T cell immunity in cases of COVID-19 and SARS, and uninfected controls. Nature 584, 457-462.
30. Lightman, S. M., Utley, A., and Lee, K. P. (2019). Survival of long-lived plasma cells (LLPC): piecing together the puzzle. Front. Immunol. 10, 965.
31. Liu, B., Li, C., Li, Z., Wang, D., Ren, X., and Zhang, Z. (2020). An entropy-based metric for assessing the purity of single cell populations. Nat. Commun. 11, 3155.
32. Lu, S., Ye, Q., Singh, D., Cao, Y., Diedrich, J. K., Yates, J. R., 3rd, Villa, E., Cleveland, D. W., and Corbett, K. D. (2021). The SARS-CoV-2 nucleocapsid phosphoprotein forms mutually exclusive condensates with RNA and the membrane-associated M protein. Nat. Commun. 12, 502.
33. Mabbott, N. A., and Gray, D. (2014). Identification of co-expressed gene signatures in mouse B1, marginal zone and B2 B-cell populations. Immunology 141, 79-95.
34. Macias-Garcia, A., Heizmann, B., Sellars, M., Marchal, P., Dali, H., Pasquali, J. L., Muller, S., Kastner, P., and Chan, S. (2016). Ikaros is a negative regulator of B1 cell development and function. J. Biol. Chem. 291, 9073-9086.
35. Moroney, J. B., Vasudev, A., Pertsemlidis, A., Zan, H., and Casali, P. (2020). Integrative transcriptome and chromatin landscape analysis reveals distinct epigenetic regulations in human memory B cells. Nat. Commun. 11, 5435.
36. Mu€ller-Winkler, J., Mitter, R., Rappe, J. C. F., Vanes, L., Schweighoffer, E., Mohammadi, H., Wack, A., and Tybulewicz, V. L. J. (2021). Critical requirement for BCR, BAFF, and BAFFR in memory B cell survival. J. Exp. Med. 218, e20191393.
37. Nelson, C. A., Lee, C. A., and Fremont, D. H. (2014). Oxidative refolding from inclusion bodies. Methods Mol. Biol. 1140, 145-157.
38. Nelson, C. A., Pekosz, A., Lee, C. A., Diamond, M. S., and Fremont, D. H. (2005). Structure and intracellular targeting of the SARS-coronavirus Orf7a accessory protein. Structure 13, 75-85.
39. Ng, K. W., Faulkner, N., Cornish, G. H., Rosa, A., Harvey, R., Hussain, S., Ulferts, R., Earl, C., Wrobel, A. G., Benton, D. J., et al. (2020). Preexisting and de novo humoral immunity to SARS-CoV-2 in humans. Science 370, 1339-1343.
40. Ochiai, K., Yamaoka, M., Swaminathan, A., Shima, H., Hiura, H., Matsumoto, M., Kurotaki, D., Nakabayashi, J., Funayama, R., Nakayama, K., et al. (2020). Chromatin protein PC4 orchestrates B cell differentiation by collaborating with IKAROS and IRF4. Cell Rep. 33, 108517.
41. Palm, A. E., and Henry, C. (2019). Remembrance of things past: long-term B cell memory after infection and vaccination. Front. Immunol. 10, 1787.
42. Patzelt, T., Keppler, S. J., Gorka, O., Thoene, S., Wartewig, T., Reth, M., Förster, I., Lang, R., Buchner, M., and Ruland, J. (2018). Foxpl controls mature B cell survival and the development of follicular and B-1 B cells. Proc. Natl. Acad. Sci. USA 115, 3120-3125.
43. Peng, Y., Mentzer, A. J., Liu, G., Yao, X., Yin, Z., Dong, D., Dejnirattisai, W., Rostron, T., Supasa, P., Liu, C., et al. (2020). Broad and strong memory CD4(+) and CD8(+) T cells induced by SARS-CoV-2 in UK convalescent individuals following COVID-19. Nat. Immunol. 21, 1336-1345.
44. Robbiani, D. F., Gaebler, C., Muecksch, F., Lorenzi, J. C. C., Wang, Z., Cho, A., Agudelo, M., Barnes, C. O., Gazumyan, A., Finkin, S., et al. (2020). Convergent antibody responses to SARS-CoV-2 in convalescent individuals. Nature 584, 437-442.
45. Rodda, L. B., Netland, J., Shehata, L., Pruner, K. B., Morawski, P. A., Thouvenel, C. D., Takehara, K. K., Eggenberger, J., Hemann, E. A., Waterman, H. R., et al. (2021). Functional SARS-CoV-2-specific immune memory persists after mild COVID-19. Cell 184, 169-183.e17.
46. Rothstein, T. L., Griffin, D. O., Holodick, N. E., Quach, T. D., and Kaku, H. (2013). Human B-1 cells take the stage. Ann. N Y Acad. Sci. 1285, 97-114.
47. Said, J. W., Hoyer, K. K., French, S. W., Rosenfelt, L., Garcia-Lloret, M., Koh, P. J., Cheng, T. C., Sulur, G. G., Pinkus, G. S., Kuehl, W. M., et al. (2001). TCL1 oncogene expression in B cell subsets from lymphoid hyperplasia and distinct classes of B cell lymphoma. Lab. Invest. 81, 555-564.
48. Sakharkar, M., Rappazzo, C. G., Wieland-Alter, W. F., Hsieh, C. L., Wrapp, D., Esterman, E. S., Kaku, Wec, A. Z., Geoghegan, J. C., McLellan, J. S., et al. (2021). Prolonged evolution of the human B cell response to SARS-CoV-2 infection. Sci. Immunol. 6, eabg6916.
49. Schena, F., Volpi, S., Faliti, C. E., Penco, F., Santi, S., Proietti, M., Schenk, U., Damonte, G., Salis, A., Bellotti, M., et al. (2013). Dependence of immunoglobulin class switch recombination in B cells on vesicular release of ATP and CD73 ectonucleotidase activity. Cell Rep. 3, 1824-1831.
50. Scholzen, T., and Gerdes, J. (2000). The Ki-67 protein: from the known and the unknown. J. Cell. Physiol. 182, 311-322.
51. Shlomchik, M. J., Aucoin, A. H., Pisetsky, D. S., and Weigert, M. G. (1987). Structure and function of anti-DNA autoantibodies derived from a single autoimmune mouse. Proc. Natl. Acad. Sci. USA 84, 9150-9154.
52. Smith, K. G., Nossal, G. J., and Tarlinton, D. M. (1995). FAS is highly expressed in the germinal center but is not required for regulation of the B-cell response to antigen. Proc. Natl. Acad. Sci. USA 92, 11628-11632.
53. Smulski, C. R., and Eibel, H. (2018). BAFF and BAFF-receptor in B cell selection and survival. Front. Immunol. 9, 2285.
54. Sokal, A., Chappert, P., Barba-Spaeth, G., Roeser, A., Fourati, S., Azzaoui, I., Vandenberghe, A., Fernandez, I., Meola, A., Bouvier-Alias, M., et al. (2021). Maturation and persistence of the anti-SARS-CoV-2 memory B cell response. Cell 184, 1201-1213.e14.
55. Stadlbauer, D., Amanat, F., Chromikova, V., Jiang, K., Strohmeier, S., Arunkumar, G. A., Tan, J., Bhaysar, D., Capuano, C., Kirkpatrick, E., et al. (2020). SARS-CoV-2 seroconversion in humans: a detailed protocol for a serological assay, antigen production, and test setup. Curr. Protoc. Microbiol. 57, e100.
56. Stuart, T., Butler, A., Hoffman, P., Hafemeister, C., Papalexi, E., Mauck, W. M., 3rd, Hao, Y., Stoeckius, M., Smibert, P., and Satij a, R. (2019). Comprehensive integration of single-cell data. Cell 177, 1888-1902.e21.
57. Uhlén, M., Fagerberg, L., Hallström, B. M., Lindskog, C., Oksvold, P., Mardinoglu, A., Sivertsson, A°., Kampf, C., Sjöstedt, E., Asplund, A., et al. (2015). Proteomics. Tissue-based map of the human proteome. Science 347, 1260419.
58. Villa, M., Gialitakis, M., Tolaini, M., Ahlfors, H., Henderson, C. J., Wolf, C. R., Brink, R., and Stockinger, B. (2017). Aryl hydrocarbon receptor is required for optimal B-cell proliferation. EMBO J. 36, 116-128.
59. Wang, C., Li, W., Drabek, D., Okba, N. M. A., van Haperen, R., Osterhaus, A. D. M. E., van Kuppeveld, F. J. M., Haagmans, B. L., Grosveld, F., and Bosch,
60. B. J. (2020). A human monoclonal antibody blocking SARS-CoV-2 infection. Nat. Commun. 11, 2251.
61. Wec, A. Z., Wrapp, D., Herbert, A. S., Maurer, D. P., Haslwanter, D., Sakharkar, M., Jangra, R. K., Dieterle, M. E., Lilov, A., Huang, D., et al. (2020). Broad neutralization of SARS-related viruses by human monoclonal antibodies. Science 369, 731-736.
62. Winkler, E. S., Bailey, A. L., Kafai, N. M., Nair, S., McCune, B. T., Yu, J., Fox, J. M., Chen, R. E., Earnest, J. T., Keeler, S. P., et al. (2020). Publisher correction: SARS-CoV-2 infection of human ACE2-transgenic mice causes severe lung inflammation and impaired function. Nat. Immunol. 21, 1470.
63. Wo hner, M., Tagoh, H., Bilic, I., Jaritz, M., Poliakova, D. K., Fischer, M., and Busslinger, M. (2016). Molecular functions of the transcription factors E2A and E2-2 in controlling germinal center B cell and plasma cell development. J. Exp. Med. 213, 1201-1221.
64. World Health Organization (2021). WHO Coronavirus (COVID-19) Dashboard. https://covid19.who.int/.
65. Yan, R., Zhang, Y., Li, Y., Xia, L., Guo, Y., and Zhou, Q. (2020). Structural basis for the recognition of SARS-CoV-2 by full-length human ACE2. Science 367, 1444-1448.
66. Yao, H., Song, Y., Chen, Y., Wu, N., Xu, J., Sun, C., Zhang, J., Weng, T., Zhang, Z., Wu, Z., et al. (2020). Molecular architecture of the SARS-CoV-2 virus. Cell 183, 730-738.e13.
67. Yi, C., Sun, X., Ye, J., Ding, L., Liu, M., Yang, Z., Lu, X., Zhang, Y., Ma, L., Gu,
68. W., et al. (2020). Key residues of the receptor binding motif in the spike protein of SARS-CoV-2 that interact with ACE2 and neutralizing antibodies. Cell. Mol. Immunol. 17, 621-630.
69. Zhao, C., Inoue, J., Imoto, I., Otsuki, T., Iida, S., Ueda, R., and Inazawa, J. (2008). POU2AF 1, an amplification target at 11q23, promotes growth of multiple myeloma cells by directly regulating expression of a B-cell maturation factor, TNERSF17. Oncogene 27, 63-75.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An antibody or antigen binding fragment comprising a heavy chain variable region and a light chain variable region, wherein the heavy chain variable region comprises a HCDR1, HCDR2, and HCDR3 having at least 80% sequence identity to the HCDR1, HCR2, HCR3 from a heavy chain variable region of a antibody clone of Table 1 and wherein the light chain variable region comprises a LCDR1, LCDR2, and LCDR3 having at least 80% sequence identity to the LCDR1, LCDR2, and LCDR3 from the light chain variable region of the same antibody clone of Table 1.

2. The antibody or antigen binding fragment of claim 1, wherein the heavy chain variable region comprises a HCDR1, HCDR2, and HCDR3 having the amino acid sequence of an of a HCDR1, HCDR2, and HCDR3 of a clone of Table 1 and wherein the light chain variable region comprises a LCDR1, LCDR2, and LCDR3 comprising the amino acid sequence of the LCDR1, LCDR2, and LCDR3 from the light chain variable region of the same clone of Table 1.

3. The antibody or antigen binding fragment of claim 1 or 2, wherein the HCDR1, HCDR2, HCDR2, LCDR1, LCDR2, and LCDR3 each comprise an amino acid sequence that has at least 80% sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

4. The antibody or antigen binding fragment of claim 1 or 2, wherein the HCDR1, HCDR2, HCDR2, LCDR1, LCDR2, and LCDR3 each comprise the amino acid sequence of an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

5. The antibody or antigen binding fragment of any one of claims 1-4, wherein the heavy chain variable region comprises an amino acid sequence with at least 80% sequence identity to a heavy chain variable region of an antibody clone of Table 1 and/or the light chain variable region comprises an amino acid sequence with at least 80% sequence identity to the light chain variable region of the same antibody clone of Table 1.

6. The antibody or antigen binding fragment of claim 5, wherein the heavy chain variable region comprises the amino acid sequence of a heavy chain variable region of an antibody clone of Table 1 and/or the light chain variable region comprises the amino acid sequence of the same antibody clone of Table 1.

7. The antibody or antigen binding fragment of any one of claims 1-6, wherein the antibody or antigen binding fragment comprises a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence with at least 80% sequence identity to an HFR1, HFR2, HFR3, and HFR4, respectively, of an antibody clone of Table 1, and the LFR1, LFR2, LFR3, and LFR4 comprises an amino acid sequence with at least 80% sequence identity to the LFR1, LFR2, LFR3, and LFR4, respectively, of the same antibody clone of Table 1.

8. The antibody or antigen binding fragment of any one of claims 1-6, wherein the HFR1, HFR2, HFR3, and HFR4 comprises the amino acid sequence of an HFR1, HFR2, HFR3, and HFR4, respectively, of an antibody clone of Table 1, and the LFR1, LFR2, LFR3, and LFR4 comprises the amino acid sequence of the LFR1, LFR2, LFR3, and LFR4, respectively, of the same antibody clone of Table 1.

9. The antibody or antigen binding fragment of any one of claims 1-8, wherein the antibody comprises a heavy chain and a light chain and wherein the heavy chain comprises an amino acid sequence with at least 70% sequence identity to a heavy chain of an antibody clone of Table 1 and the light chain comprises an amino acid sequence with at least 70% sequence identity to the light chain of the same antibody clone of Table 1.

10. The antibody or antigen binding fragment of claim 9, wherein the antibody comprises a heavy chain and a light chain and wherein the heavy chain comprises the amino acid sequence of an antibody clone of Table 1 and the light chain comprises the amino acid sequence of the same antibody clone of Table 1.

11. The antibody of any one of claims 1-10, wherein the antibody is human, chimeric, or humanized.

12. The antibody or antigen-binding fragment of any one of claims 1-11, wherein the antibody, or antigen binding fragment binds a SARS-CoV-2 protein with a K_{D} of about 10⁻⁶ nM to about 10⁻¹² pM.

13. The antibody or antigen binding fragment of any one of claims 1-12, wherein the antibody is a neutralizing antibody.

14. The antibody or antigen binding fragment of any one of claims 1-13, wherein the antibody is a human antibody, humanized antibody, recombinant antibody, chimeric antibody, an antibody derivative, a veneered antibody, a diabody, a monoclonal antibody, a single domain antibody, or a single chain antibody.

15. The antigen binding fragment of any one of claims 1-13, wherein the antigen binding fragment is a single chain variable fragment (scFv), F(ab′)₂, Fab′, Fab, Fv, or rIgG.

16. A polypeptide comprising the antigen binding fragment of any one of claims 1-15.

17. The polypeptide of claim 16, wherein the polypeptide comprises at least two antigen binding fragments, wherein each antigen binding fragment is independently selected from an antigen binding fragment of any one of claims 1-15.

18. The polypeptide of claim 16 or 17, wherein the polypeptide is multivalent.

19. The polypeptide of any one of claims 16-18, wherein the polypeptide is bispecific.

20. A composition comprising the antibody or antigen binding fragment of any one of claims 1-19.

21. The composition of claim 20, wherein the composition comprises a pharmaceutical excipient.

22. The composition of claim 20 or 21, wherein the composition further comprises an adjuvant.

23. The composition of any one of claims 20-22, wherein the composition is formulated for parenteral, intravenous, subcutaneous, intramuscular, or intranasal administration.

24. The composition of any one of claims 1-23, wherein the composition comprises at least two antibodies or antigen binding fragments.

25. One or more nucleic acids encoding the antibody or antigen binding fragment of any one of claims 1-15 or the polypeptide of claim 19.

26. A nucleic acid encoding an antibody heavy chain, wherein the nucleic acid has at least 70% sequence identity to one of SEQ ID NOS:1621-1710 or 2707-2755.

27. A nucleic acid encoding an antibody light chain, wherein the nucleic acid has at least 70% sequence identity to one of SEQ ID NOS:1711-1800 or 2756-2804.

28. A vector comprising the nucleic acid(s) of any one of claims 25-27.

29. A host cell comprising the nucleic acid of any one of claims 25-27 or the vector of claim 28.

30. The host cell of claim 29, wherein the host cell is a human cell, B cell, T cell, Chinese hamster ovary, NS0 murine myeloma cell, or PER.C6 cell.

31. A method of a making a cell comprising transferring the nucleic acid(s) of any one of claims 25-27 or the vector of claim 28 into a cell.

32. The method of claim 31, wherein the method further comprises culturing the cell under conditions that allow for expression of a polypeptide from the nucleic acid.

33. The method of claim 32, wherein the method further comprising isolating the expressed polypeptide.

34. The method of any one of claims 31-33, wherein the cell is a human cell, B cell, T cell, Chinese hamster ovary, NS0 murine myeloma cell, or PER.C6 cell.

35. A method for producing a polypeptide comprising transferring the nucleic acid(s) of any one of claims 25-27 or the vector of claim 28 into a cell and isolating polypeptides expressed from the nucleic acid.

36. The method of claim 35, wherein the cell is a human cell, B cell, T cell, Chinese hamster ovary, NS0 murine myeloma cell, or PER.C6 cell.

37. A method for treating or preventing a coronavirus infection in a subject, the method comprising administering to the subject, the antibody or antigen binding fragment of any one of claims 1-15, the polypeptide of claim 19, or the host cell of claim 29.

38. The method of claim 37, wherein the subject is a human subject.

39. The method of claim 37 or 38, wherein the coronavirus infection is SARS-CoV-2.

40. The method of claim 37 or 38, wherein the subject has one or more symptoms of a coronavirus infection.

41. The method of claim 37 or 38, wherein the subject does not have any symptoms of a coronavirus infection.

42. The method of any one of claims 37-41, wherein the subject has been diagnosed with a coronavirus infection.

43. The method of any one of claims 37-41, wherein the subject has not been diagnosed with a coronavirus infection.

44. The method of any one of claims 37-43, wherein the subject has been previously vaccinated for coronavirus.

45. The method of any one of claims 37-43, wherein the subject has not been previously vaccinated for coronavirus.

46. The method of any one of claims 37-45, wherein the antibody, antigen binding fragment, polypeptide, or cell is administered by parenteral, intravenous, subcutaneous, intramuscular, or intranasal administration.

47. The method of any one of claims 37-43, wherein the subject has been previously treated for a coronavirus infection.

48. The method of any one of claims 37-47, wherein the subject is administered an additional therapeutic.

49. The method of claim 48, wherein the additional therapeutic comprises a steroid or an anti-viral therapeutic.

50. The method of claim 49, wherein the additional therapeutic comprises dexamethasone or remdesivir.

51. A method for evaluating a sample from a subject, the method comprising contacting a biological sample from the subject, or extract thereof, with at least one antibody, antigen binding fragment, or polypeptide of any one of claims 1-19.

52. The method of claim 51, wherein the at least one antibody, antigen binding fragment, or polypeptide is operatively linked to a detectable label.

53. The method of claim 51 or 52, wherein the method further comprises incubating the antibody, antigen binding fragment, or polypeptide under conditions that allow for the binding of the antibody, antigen binding fragment, or polypeptide to antigens in the biological sample or extract thereof.

54. The method of any one of claims 51-53, wherein the method further comprises detecting the binding of an antigen to the antibody, antigen binding fragment, or polypeptide.

55. The method of any one of claims 51-54, wherein the method further comprises contacting the biological sample with at least one capture antibody, antigen, or polypeptide.

56. The method of claim 55, wherein the at least one capture antibody, antigen binding fragment, or polypeptide comprises at least one antibody of claims 1-19.

57. The method of claim 55 or 56, wherein the capture antibody is linked to a solid support.

58. The method of any one of claims 51-57, wherein the biological sample comprises a blood sample, urine sample, fecal sample, or nasopharyngeal sample.

59. A method for diagnosing a SARS-CoV-2 infection in a subject, the method comprising contacting a biological sample from the subject, or extract thereof, with at least one antibody, antigen binding fragment, or polypeptide of any one of claims 1-19.

60. The method of claim 59, wherein the at least one antibody, antigen binding fragment, or polypeptide is operatively linked to a detectable label.

61. The method of claim 59 or 60, wherein the method further comprises incubating the antibody, antigen binding fragment, or polypeptide under conditions that allow for the binding of the antibody, antigen binding fragment, or polypeptide to antigens in the biological sample or extract thereof.

62. The method of any one of claims 59-61, wherein the method further comprises detecting the binding of an antigen to the antibody, antigen binding fragment, or polypeptide.

63. The method of any one of claims 59-62, wherein the method further comprises contacting the biological sample with at least one capture antibody, antigen, or polypeptide.

64. The method of claim 63, wherein the at least one capture antibody, antigen, or polypeptide comprises at least one antibody, antigen, or polypeptide of claims 1-19.

65. The method of claim 63 or 64, wherein the capture antibody is linked to a solid support.

66. The method of any one of claims 59-65, wherein the biological sample comprises a blood sample, urine sample, fecal sample, or nasopharyngeal sample.
